(19) United States
(12) Patent Application Publication
(10) Pub. No.: US 2009/0247734 A1
(43) Pub. Date: Oct. 1, 2009

(54) Chemically Derivatized CD4 and Uses Thereof

(76) Inventors: Wayne A. Hendrickson, New York, NY (US); Hui Xie, New York, NY (US); Amos B. Smith, Merion, PA (US); Danny Ng, Daly City, CA (US)

Correspondence Address: COOPER & DUNHAM, LLP, 30 Rockefeller Plaza, 20th Floor, NEW YORK, NY, 10112, US

(21) Appl. No.: 12/086,675

(22) PCT Filed: Dec. 14, 2006
(86) PCT No.: PCT/US06/47907

§ 371 Date: Dec. 17, 2008

Related U.S. Application Data
(60) Provisional application No. 60/750,464, filed on Dec. 14, 2005.
(60) Provisional application No. 60/789,467, filed on Apr. 4, 2006.

(51) Int. Cl. C07K 14/73; G06G 7/48

(52) U.S. Cl. 530/380; 703/11

(57) ABSTRACT

This invention provides two soluble polypeptides which comprise a portion of CD4 comprising all HIV gp120-binding epitopes present on intact CD4, wherein the polypeptide has a cysteine substitution at a residue which, in intact CD4, interfaces with HIV gp120. This invention also provides a method for making a derivatized soluble polypeptide and a method for obtaining a structural model useful in the design of an agent for inhibiting CD4 binding to HIV gp120.

The invention disclosed herein was made with United States government support under grant number GM56550 from the National Institutes of Health. Accordingly, the United States government has certain rights in this invention.

Throughout this application, various publications are referenced. Full bibliographic citations for these publications are found at the end of the specification immediately preceding the claims. The disclosures of these publications in their entireties are hereby incorporated by reference into this application in order to more fully describe the state of the art known to those skilled therein as of the date of the invention described and claimed herein.

### BACKGROUND OF THE INVENTION

Human Immunodeficiency Virus (HIV) is the primary cause of Acquired Immunodeficiency Syndrome (AIDS) (Barre-Sinoussi, Chermann et al. 1983; Gallo, Salahuddin et al. 1984). Today, twenty antiretroviral drugs have been approved by FDA for clinical treatment of AIDS (De Clercq 2005). Most of them target either the reverse transcriptase or the protease of HIV with one exception: enfuvirtide that targets virus fusion. Although the mortality of the HIV-infected patients has been largely decreased by HAART (highly active antiretroviral therapy) (Richman 2001), emergency of drug-resistant virus and drug toxicity problems demand the search for novel antiretroviral drugs.

As the first step of HIV life cycle that precedes cellular infection, the elements of virus entry are attractive antiviral targets. The entry of the virus is mediated by the specific interactions between viral envelope glycoproteins and host cell surface receptors. The virus envelope glycoprotein complex is a trimer (Chan, Fass et al. 1997; Tan, Liu et al. 1997; Weissenhorn, Dessen et al. 1997) consisting of three pairs of gp41 and gp120, both derived by cleavage of precursor gp160 (Allan, Coligan et al. 1985; Robey, Safai et al. 1985). gp41 is a membrane protein, and gp120 attaches to the virion through non-covalent interaction with gp41 (Helseth, Olshevsky et al. 1991). Sequence analysis of gp120s from HIV-1, HIV-2 and SIVs identifies five conserved regions (C1 to C5) and five variable regions (V1 to V5) (Starcich, Hahn et al. 1986; Modrow, Hahn et al. 1987).

HIV first attaches to host cell surface through gp120's recognition of CD4, a glycoprotein on the surface of the host cell (Dalgleish, Beverley et al. 1984; Klatzmann, Champagne et al. 1984). The molecular details of this interaction have been revealed by X-ray crystal structures of various core gp120 proteins (Kwong, Wyatt et al. 1998; Kwong, Wyatt et al. 2000; Huang, Tang et al. 2005) from three different HIV strains in complex with D1D2 (the first two immunoglobulin-like domains of sCD4) and a Fab fragment of antibody, 17B or X5. In these complexes, D1 domain of CD4 binds into a depression on the core gp120 formed by all three domains of gp120 including inner domain, outer domain, and a bridging β-sheet structure that appears to require the interaction of CD4 for its integrity. A separate thermodynamic analysis also shows a unusually large structural rearrangement of both gp120 and the core gp120 upon CD4 binding (Myszka, Sweet et al. 2000). In contrast, the structure of D1D2 (Ryu, Kwong et al. 1990; Wang, Yan et al. 1990; Ryu, Truneh et al. 1994; Wu, Kwong et al. 1997) is essentially unchanged in the presence of gp120. The primary binding site of CD4 is located in the second complementarity-determining region (CDR2) of D1 domain. Although twenty-two residues of CD4 are involved in gp120 binding, 63% of all contacts come from residue 40-48 of CD4. Among them, Phe43 alone contributes 23% of the total interactions. Another CD4 determinant at the interface is residue Arg59, contributing two hydrogen-bonds with gp120 (Kwong, Wyatt et al. 1998). At the interface of gp120-CD4 in all structures, a deep hydrophobic cavity enclosed by conserved gp120 residues has been identified and Phe43 is the only CD4 residue that contacts it (FIG. 1). Hence, this pocket has been referred as “Phe43 Cavity” (Kwong, Wyatt et al. 1998; Kwong, Wyatt et al. 2000). Phe43 cavity has been shown to be absent in the structure of a unliganded core SIV gp120 (Chen, Vogan et al. 2005). This pocket unique to CD4-bound conformation of gp120 has been suggested as a potential target for therapeutic intervention (Kwong, Wyatt et al. 1998; Wyatt, Kwong et al. 1998; Kwong, Wyatt et al. 2000).

CD4 binding induces extensive structural rearrangements in gp120, resulting the exposure of binding surface for a second host cell chemokine receptor, CCR5 or CXCR4 (Trkola, Dragic et al. 1996; Wu, Gerard et al. 1996). The following engagement of gp120 with the chemokine receptor triggers further conformational changes in gp120-associated gp41, which then releases its “fusion peptide” (Kowalski, Potz et al. 1987) for insertion into target cell membranes and ultimately mediates virus-cell membrane fusion (Lu, Blacklow et al. 1995; Chan, Fass et al. 1997; Weissenhorn, Dessen et al. 1997).

Entry inhibitors target one of the following steps in the virus entry: viral attachment by gp120-CD4 interaction, coreceptor binding, and fusion between virus and host cell (Kilby and Eron 2003). Enfuvirtide, a small peptide derived from gp41, is the only available entry drug targeting viral fusion step by inhibiting the formation of the “six-helix bundle” during fusion (Furuta, Wild et al. 1998). There are also many drug candidates under clinical development, which target the other two steps of viral entry. Most of the inhibitors of coreceptor binding are small molecules that bind either CCR5 or CXCR4 by mimicking the natural ligands of the receptors; on the contrary, most of potent gp120-CD4 inhibitors identified to data are proteins or peptides (Vermeire and Schols 2005).

A large fraction of gp120-CD4 inhibitors are gp120-directed while some of them, such as PRO 2000, a naphthalene polyanion that binds CD4, CD3, and CD8 (Rusconi, Moonis et al. 1996, Milligan, Chu et al. 2004) are CD4-directed. Three strategies have been used to develop gp120-directed inhibitors: rational design of CD4 mimics, peptide phage display, and high-throughput screening. CD4-based gp120-targeting inhibitors range from gp120 antibody IgG1 b12 (Burton, Pyati et al. 1994), fusion protein of CD4 with IgG₂ (PRO 542) (Allaway, Davis-Bruno et al. 1995) to CD4 miniproteins, which are scorpion toxin-based mimetics that have CDR2 loop of CD4 transplanted into toxin scaffold. The most successful inhibitor of the latter kind is CD4M33, a 27-amino acid mimetic that inhibit the interaction of gp120 and CD4 at nanomolar concentration (Martin, Stricher et al. 2003). This mimetic uses a bi-phenyl group instead of phenyl at the position corresponding to Phe43 of CD4 and structure of CD4M33 in complex with gp120:17b reveals the binding site of the additional phenyl as the Phe43 cavity (Huang, Stricher et al. 2005). There is also sCD4-17b, a single-chain chimeric protein of D1D2 and 17b, capable of targeting both CD4 and co-receptor sites on gp120 (Dey, Del Castillo et al. 2003). Random peptide libraries screening based on phage display has led to the discovery of a peptide 12p1 that blocks gp120's interaction with both CD4 and 17b with micromolar IC₅₀ (Ferrer and Harrison 1999). Screening of extracts from cultured cyanobacteria identified cyanovirin-N (Boyd, Gustafson et al. 1997), an 11-kDa protein, which inhibits both CD4 and coreceptor by interacting with high-mannose glycans on gp120. Screening of small compound library, however, has yet to identify any potent candidate. BMS-378806, a small molecule with high anti-entry activity, was initially identified by a viral-infection-based screen and had been shown to block CD4-gp120 interaction by binding gp120 (Guo, Ho et al. 2003; Lin, Blair et al. 2003; Wang, Zhang et al. 2003). New evidence, however, indicated that it exerts its inhibitory function on entry through blocking the CD4 induction of fusion-driving conformation in gp41 (Si, Madani et al. 2004). Study on BMS-378806 escape mutants of gp120 suggests a possible binding site of the compound near Phe43 cavity (Madani, Perdigoto et al. 2004).

The difficulty in identifying a small molecule inhibiting gp120-CD4 interaction with sub-micromolar IC₅₀ is not surprising. Protein-protein interaction has long known to be attractive but not straightforward drug target due to rather flat features of protein-protein interface (Cochran 2000). Interfacial hydrophobic pocket like Phe43 cavity in gp120, however, could be binding site for small molecules that block protein-protein binding either by direct steric effect or through allosteric mechanism. A good example can be found in the case of rhinoviruse, where compounds targeting the viral protein 1 (VP1) bind into the hydrophobic pocket just beneath the canyon floor, which is important in cellular receptor binding (Chapman, Minor et al. 1991; Zhang, Nanni et al. 1993).

Conventional high-throughput screening is only strong in identifying medium-affinity (low μM to nM) compounds, but relatively small size of Phe43 cavity (152 Å³) (Kwong, Wyatt et al. 1998) as well as large unfavorable entropic change involved in forming this cavity (Myszka, Sweet et al. 2000), have made identification of small molecules targeting this site with medium-high affinity extremely difficult.

### SUMMARY OF THE INVENTION

This invention provides a soluble polypeptide consisting of a portion of CD4 comprising all HIV gp120-binding epitopes present on intact CD4, wherein the polypeptide has a cysteine substitution at a residue which, in intact CD4, interfaces with HIV gp120.

This invention provides a soluble polypeptide comprising (i) a portion of CD4 comprising all HIV gp120-binding epitopes present on intact CD4, wherein the polypeptide has a cysteine substitution at a residue which, in intact CD4, interfaces with HIV gp120, and (ii) a chemical moiety bound to the CD4 portion at the cysteine substitution via a thiol bond.

This invention provides a method for making a derivatized soluble polypeptide comprising contacting, under suitable conditions, (a) a thiol-reactive reagent with (b) a portion of CD4 comprising all HIV gp120-binding epitopes present on intact CD4, wherein the polypeptide has a cysteine substitution at a residue which, in intact CD4, interfaces with HIV gp120.

This invention provides a method for obtaining a structural model useful in the design of an agent for inhibiting CD4 binding to HIV gp120 comprising (a) identifying a soluble polypeptide of claim 5 which binds to HIV gp120 with an affinity comparable to or greater than the affinity with which intact CD4 binds to HIV gp120; and (b) obtaining a three-dimensional structure of the identified polypeptide while it is bound to HIV gp120, thereby obtaining a structural model useful in the design of an agent for inhibiting CD4 binding to HIV gp120.

### BRIEF DESCRIPTION OF THE FIGURES

FIG. 1

This figure shows a design of modified D1D2F43C for targeting the gp120 Phe43 cavity.

FIG. 2

This figure shows the modification of F43C of D1D2 by haloacetamides, halopropanones or 5-nitro-2-pyridinesulfenyl reagents.

FIG. 3

This figure shows representative curves for the inhibition of gp120-CD4 binding by D1D2F43C derivatives. Legend: ▪=D1D2 control; □=D1D2F43C-Iodoacetamide; =D1D2F43C-10; ◯=D1D2F43C; ♦=D1D2F43C-19; ⋄=D1D2F43C-DN52.

FIG. 4

This figure shows distribution of the IC₅₀ values of the D1D2F43C derivatives derived from both libraries.

FIGS. 5A & 5B

These figures show comparisons of IC₅₀ values of D1D2F43C derivatives on binding of D1D2 to YU2 FL gp120 to that on the binding of D1D2 to YU2 375S/W & 257T/S gp120.

FIG. 6

This figure shows the correlation between the sizes of the compounds and the folds of IC₅₀ values of their derivatives increased from wild type gp120 to S375W/T257S gp120

FIG. 7

This figure shows probing of the Phe-43 pocket: binding of chemically modified CD4 to HIV gp120. Phe-43 of CD4 replaced by Cys-43. Chemical modification of Cys-43 by S-alkylation with bromoacetamides. Effects of different substituents at position 43 on gp120 binding. X-ray structures of derivatized CD4-gp120 complexes. Over 100 bromoacetamides have been prepared in the Smith laboratory.

FIG. 8

This figure shows probing of the Phe-43 pocket: binding of chemically modified CD4 to HIV gp120. The synthesis of bromoacetamides-1-7 steps from commercially available starting material.

FIG. 9

This figure shows binding of chemically modified CD4 to HIV gp120.

FIG. 10

This figure shows binding of chemically modified CD4 to HIV gp120: structure-affinity relationship. Branching at P5 (except cyclohexane and aromatic group) disfavors binding.

FIG. 11

This figure shows binding of chemically modified CD4 to HIV gp120: structure-affinity relationship. Electronic effect and substitution pattern.

FIG. 12

This figure shows binding of chemically modified CD4 to HIV gp120: structure-affinity relationship. Other aromatic groups shown. With few exceptions, most chemically modified CD4 have similar binding affinities as native CD4 (A “flat” SAR). Phe43 cavity is able to accommodate changes in substituents.

FIGS. 13 & 14

These figures show binding of chemically modified CD4 to HIV gp120: X-ray structures of derivatized CD4-gp120 complexes.

FIG. 15

These figures show binding of chemically modified CD4 to HIV gp120: X-ray structures of derivatized CD4-gp120 complexes. Internal plasticity of the cavity: volume of Phe43 pocket expands to accommodate structural changes.

FIG. 16

This figure shows current design and synthetic efforts such as introduction of additional non-covalent interactions: newly discovered H₂O sites; extension into water channels; and crystallization and structural determination of additional complexes.

FIG. 17

This figure shows X-ray diffraction-derived structural data for complexes of derivatized CD4 fragments and gp120, namely HX-SNS-10.

FIG. 18

This figure shows X-ray diffraction-derived structural data for complexes of derivatized CD4 fragments and gp120, namely HX-SNS-14.

FIG. 19

This figure shows X-ray diffraction-derived structural data for complexes of derivatized CD4 fragments and gp120, namely HX-SNS-40.

FIG. 20

This figure shows X-ray diffraction-derived structural data for complexes of derivatized CD4 fragments and gp120, namely HX-DN-234.

FIG. 21

This figure shows a summary of HXBc2 core gp120:17b:CD4-derivative complexes (abbreviated as HX-compound) in comparison with the wild type gp120:17b:CD4 complex (HX-WT) (Kwong et al. 2000). Ribbon diagram of gp120 bound by a chemically derivatized CD4-D1D2 protein. The chemical group attached to Cα of residue 43 of CD4 is represented as “R”, which is positioned right in the Phe43 cavity of gp120. Fab fragment of 17b is removed from the figure for clarity.

FIG. 22

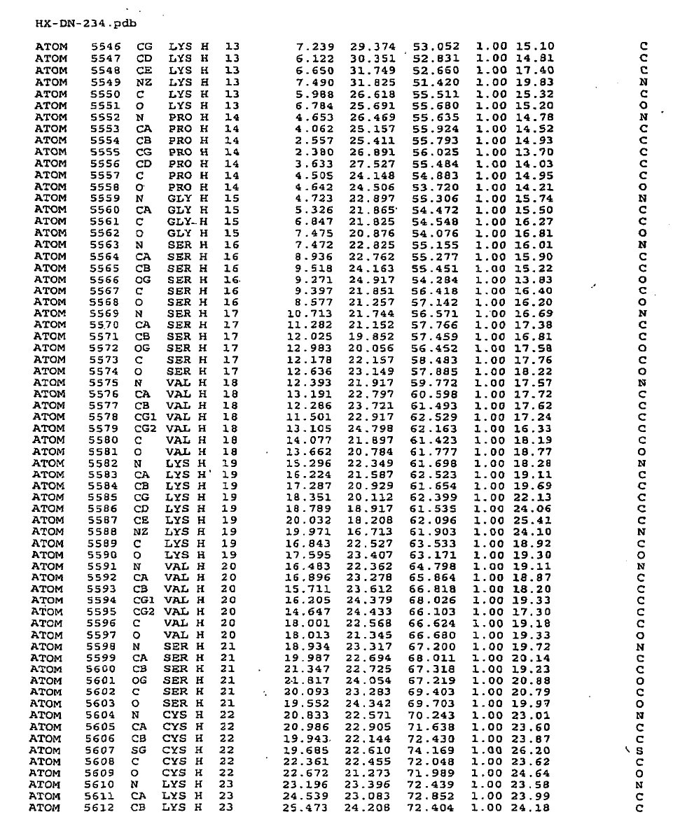

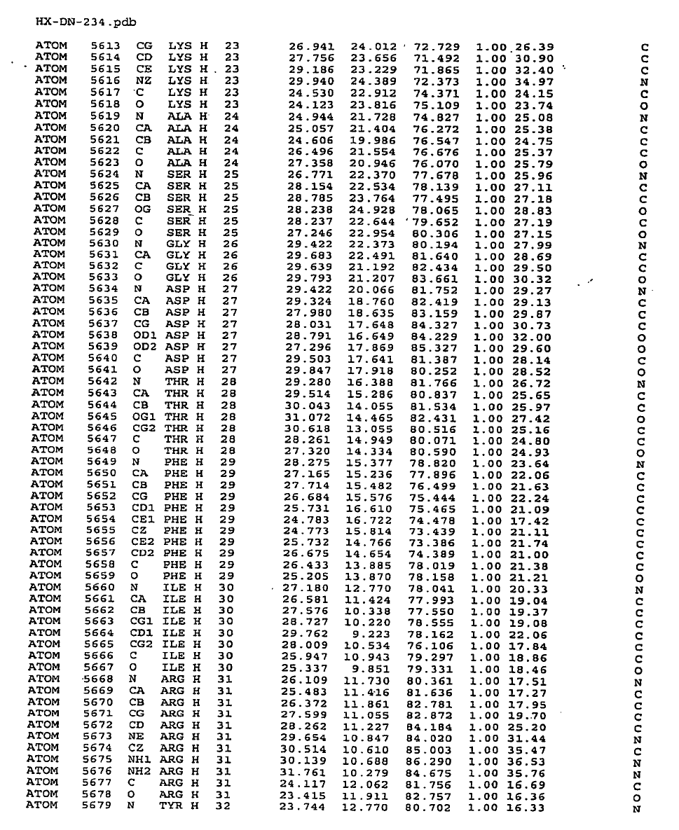

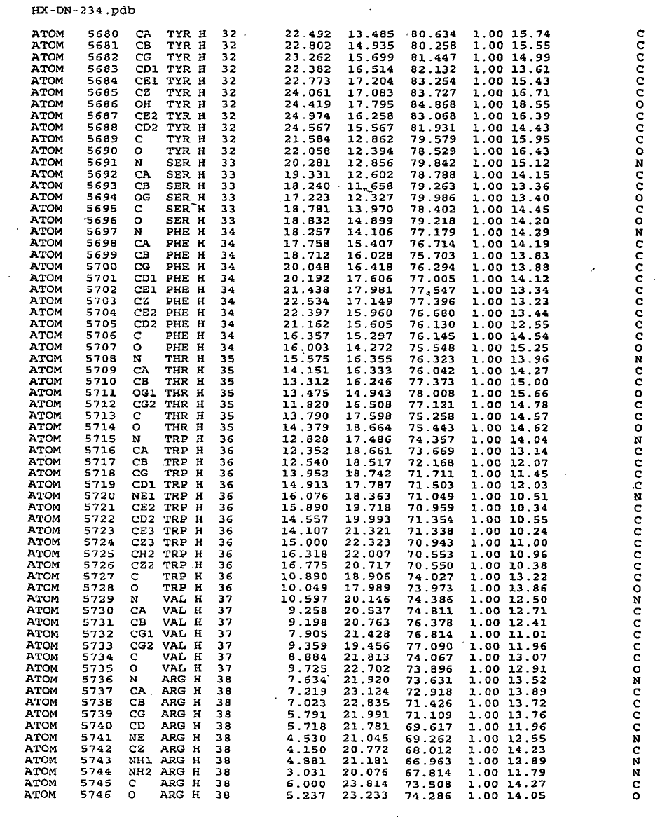

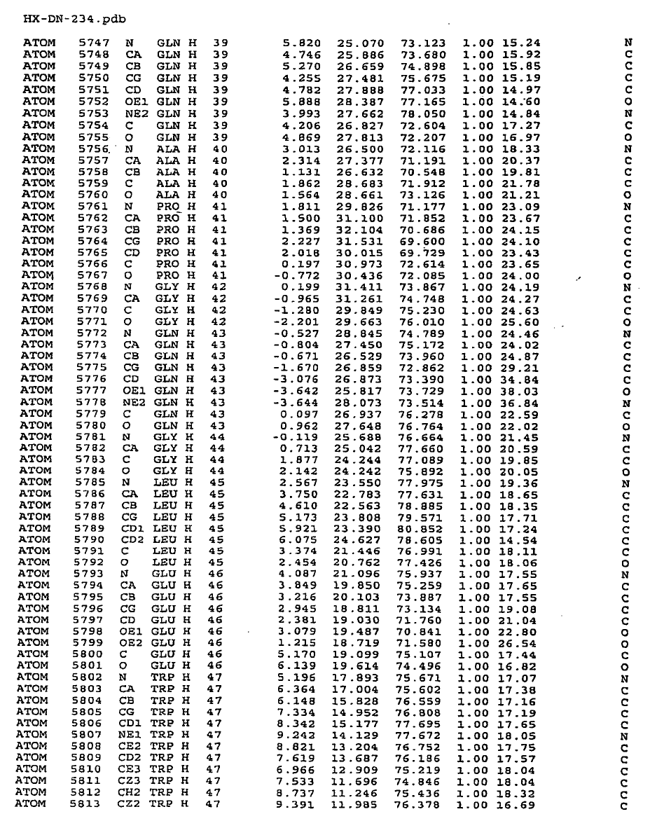

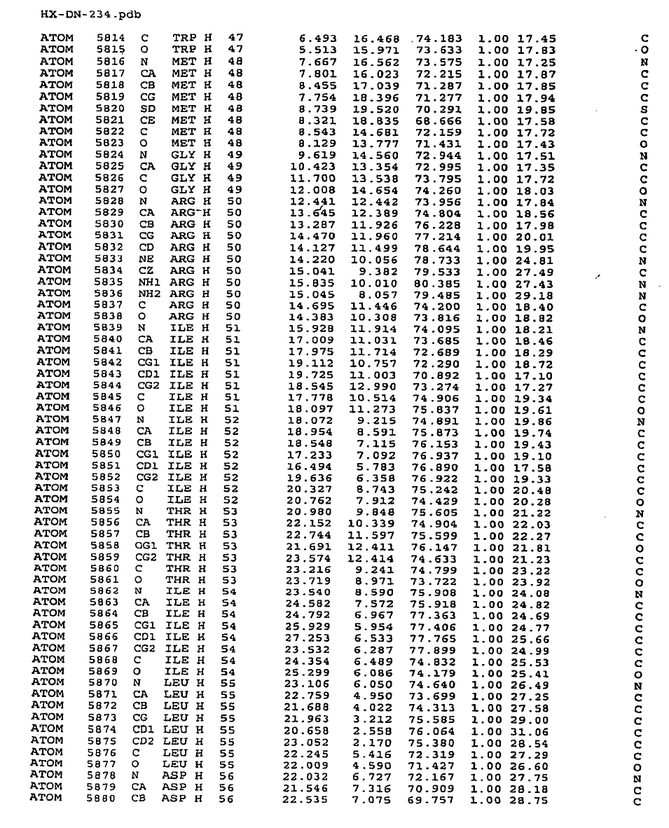

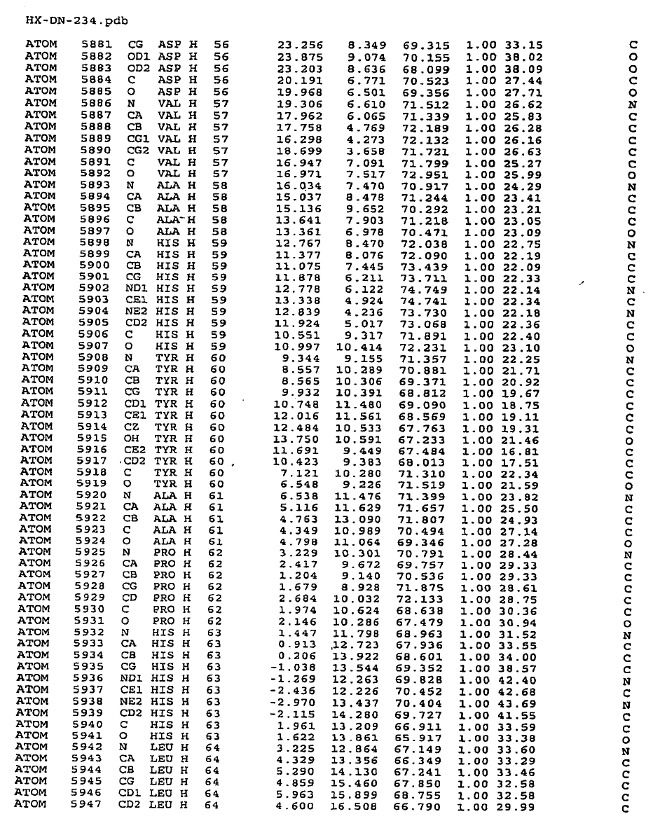

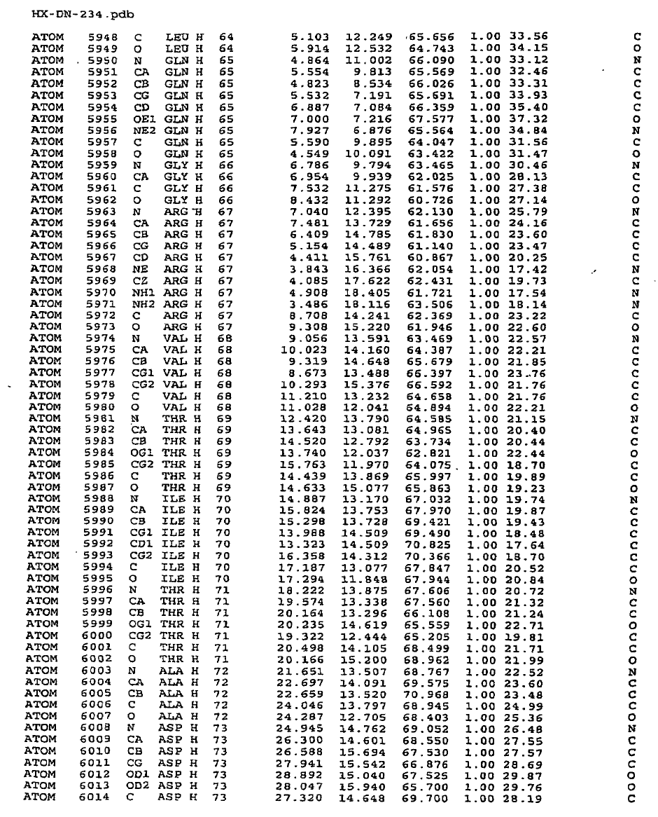

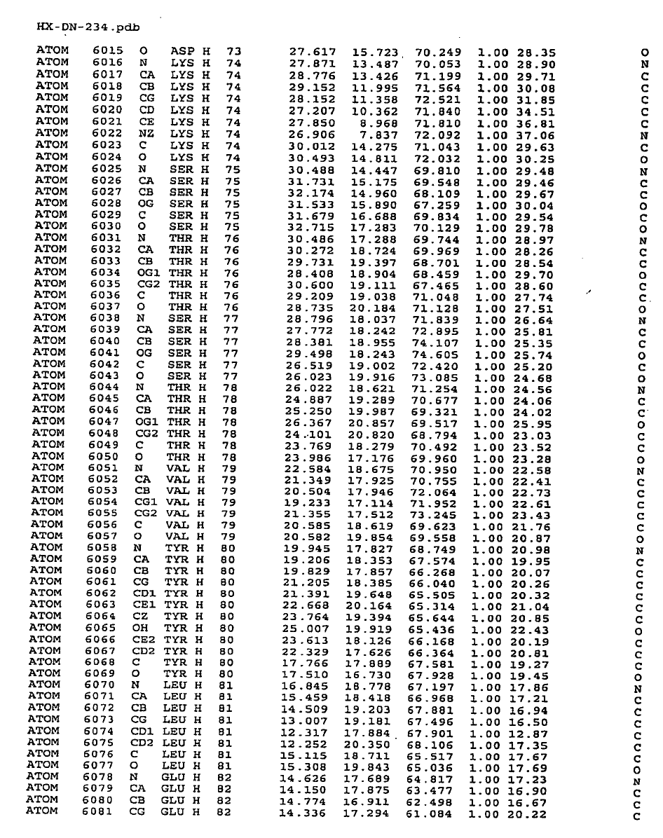

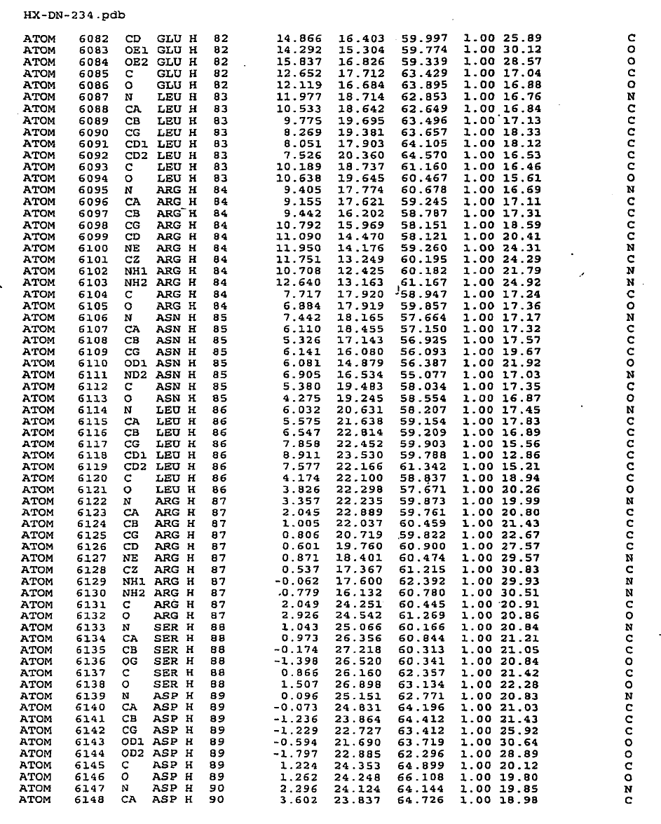

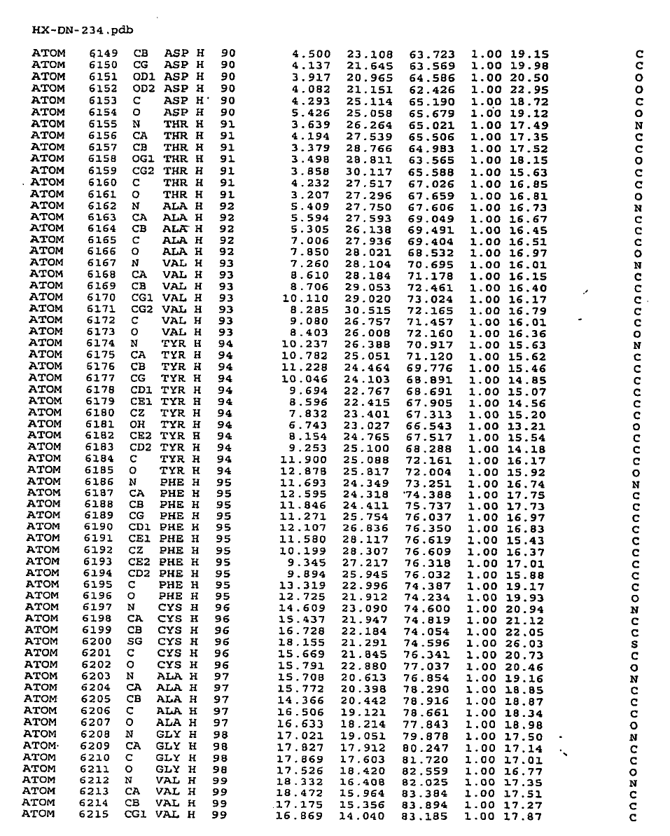

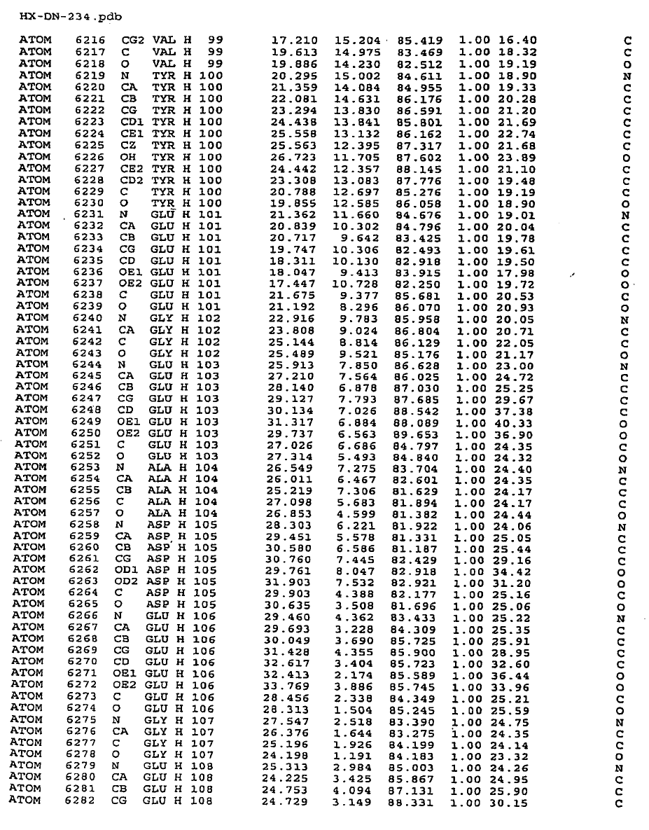

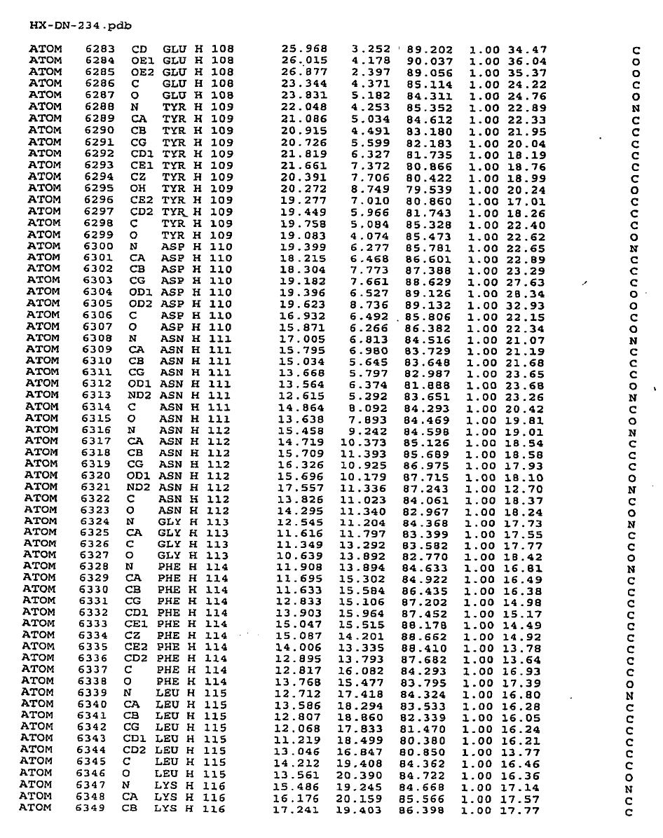

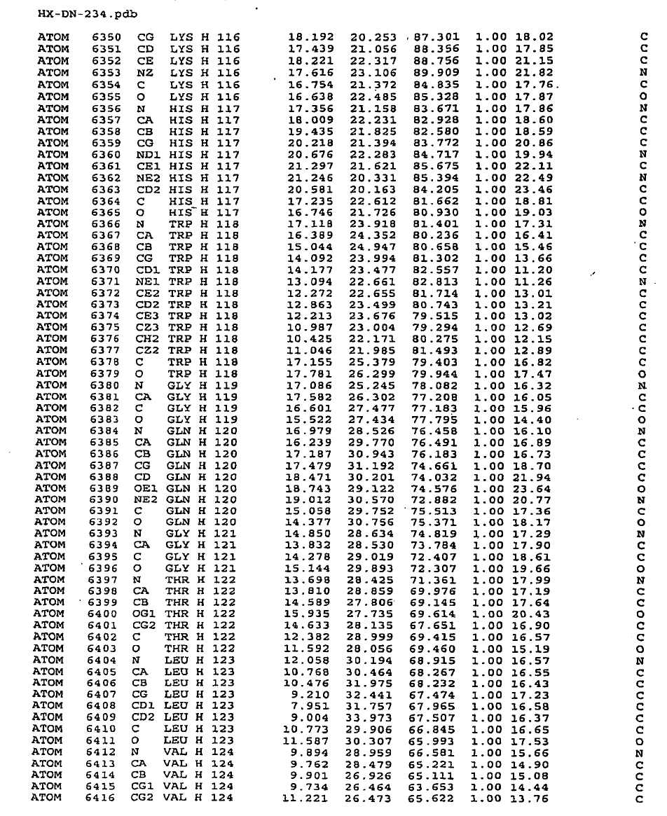

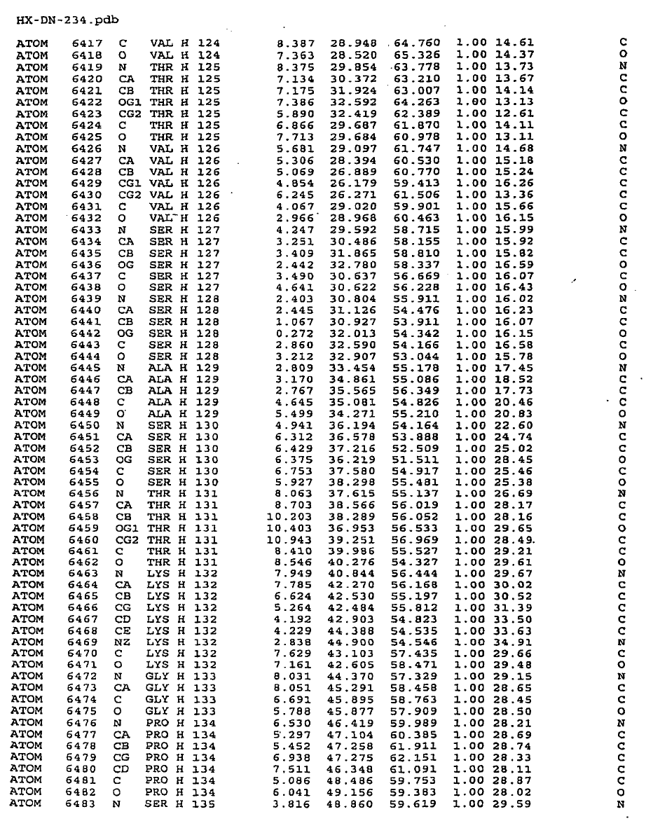

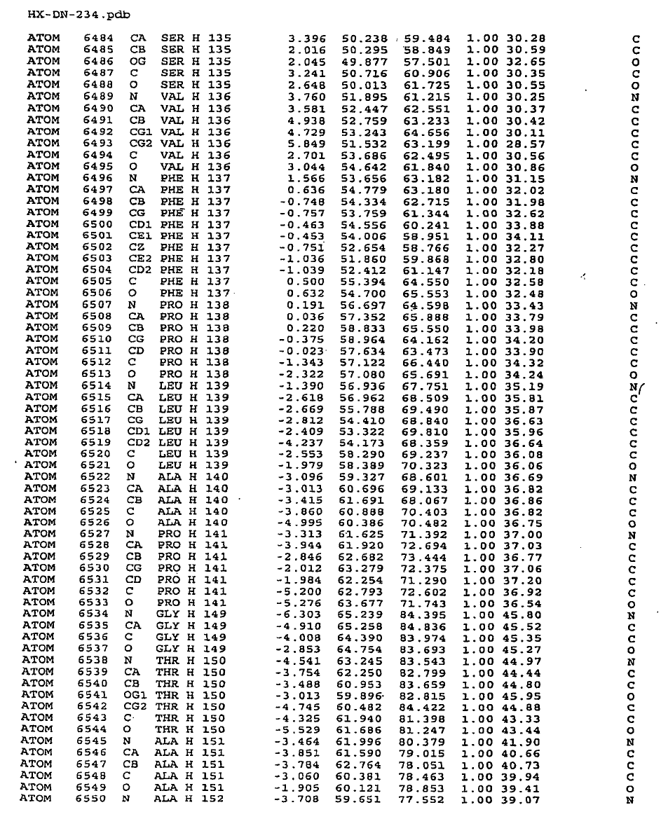

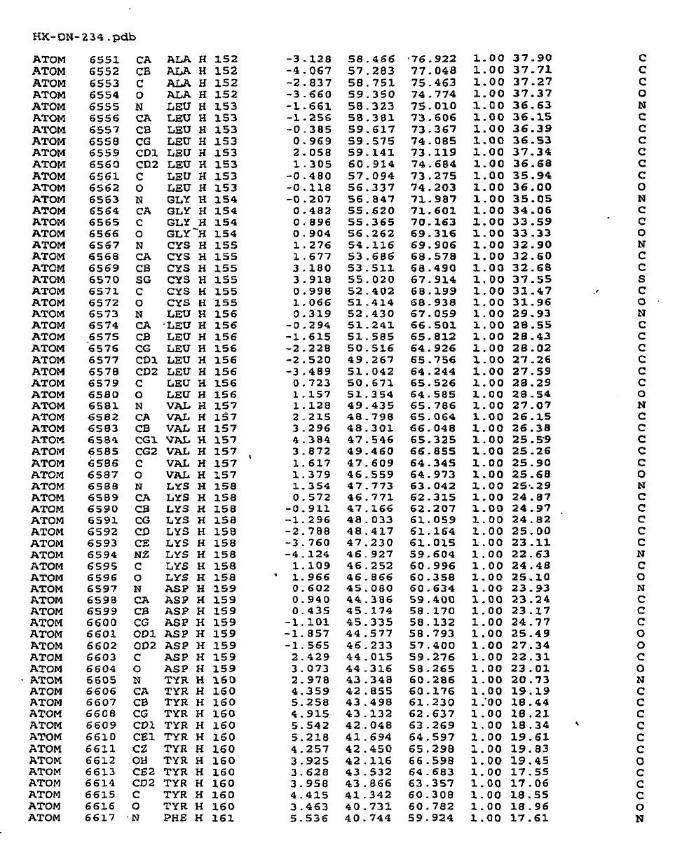

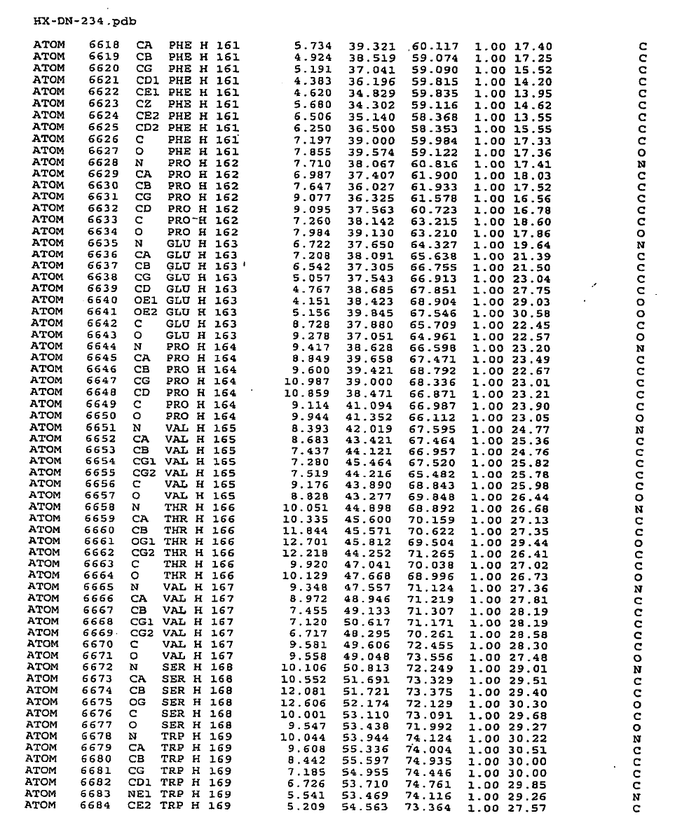

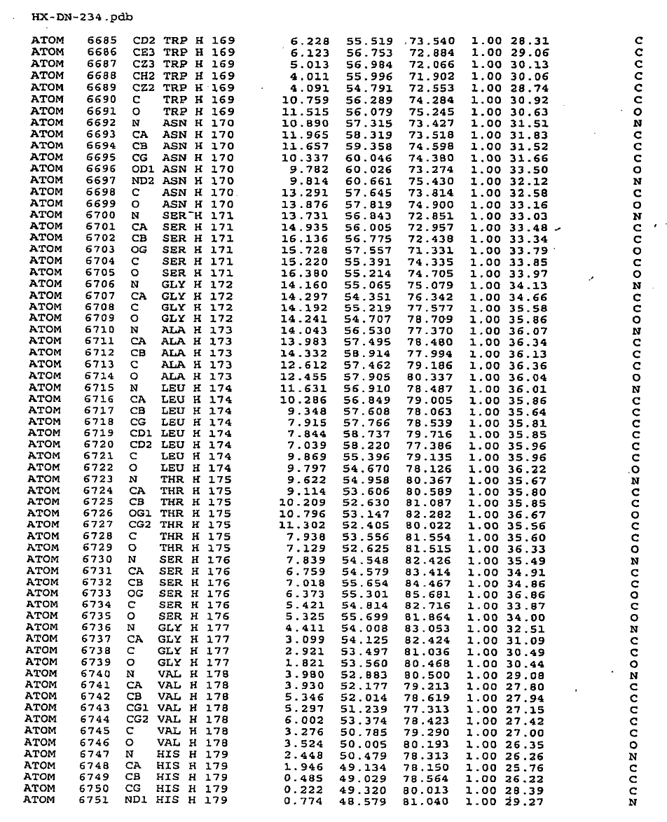

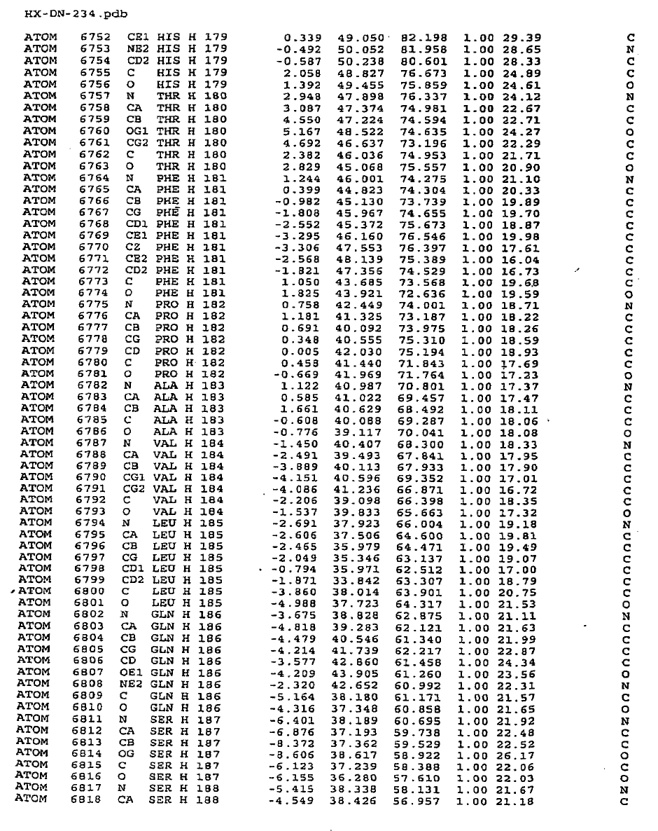

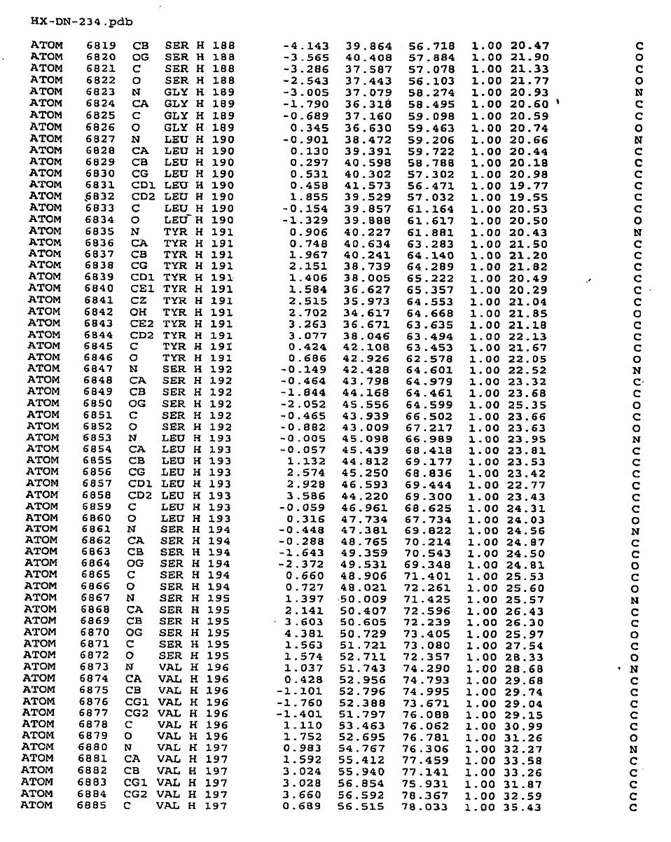

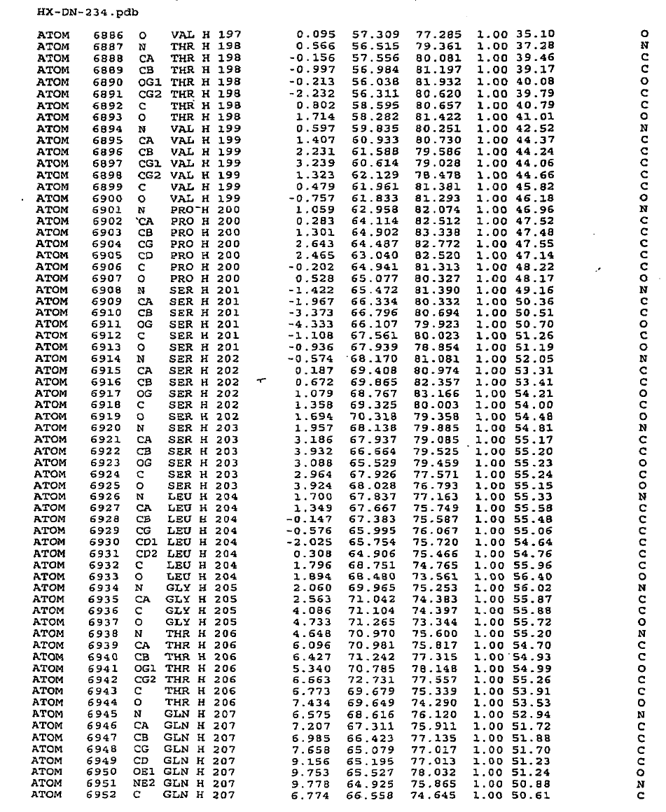

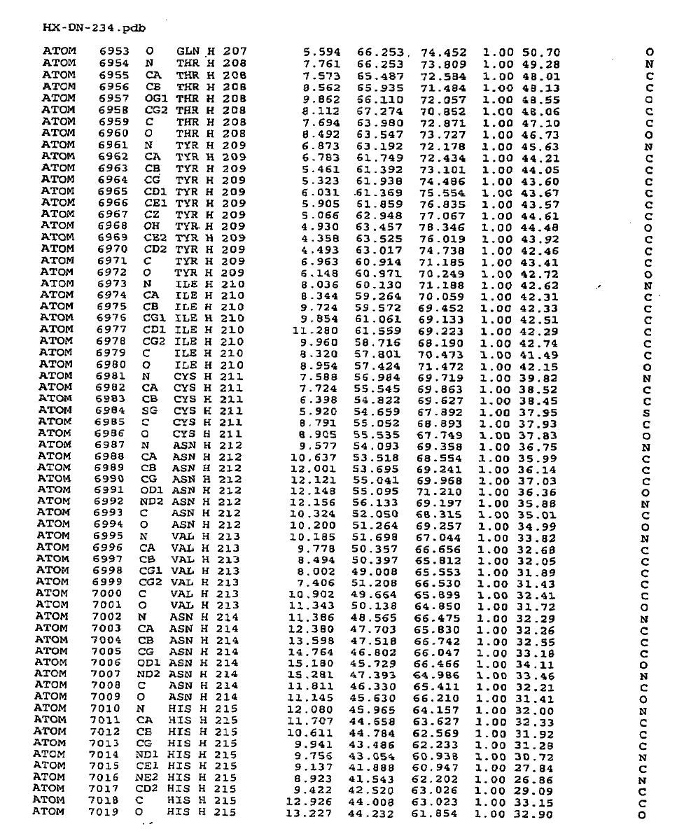

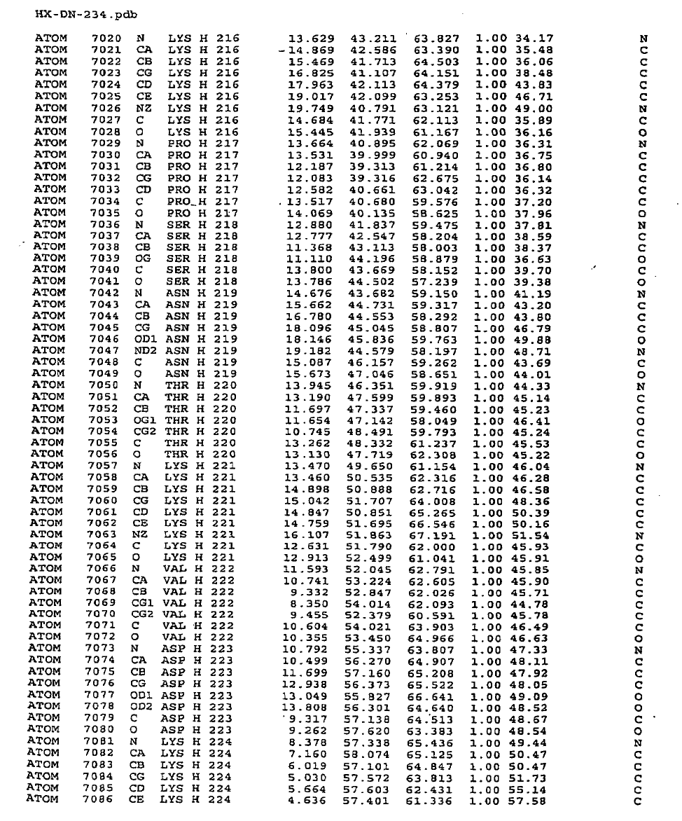

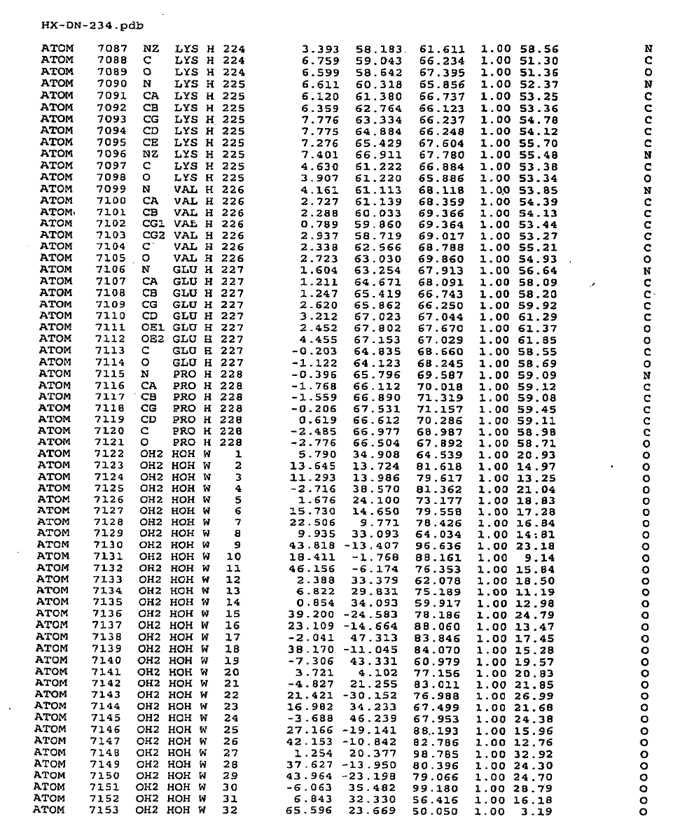

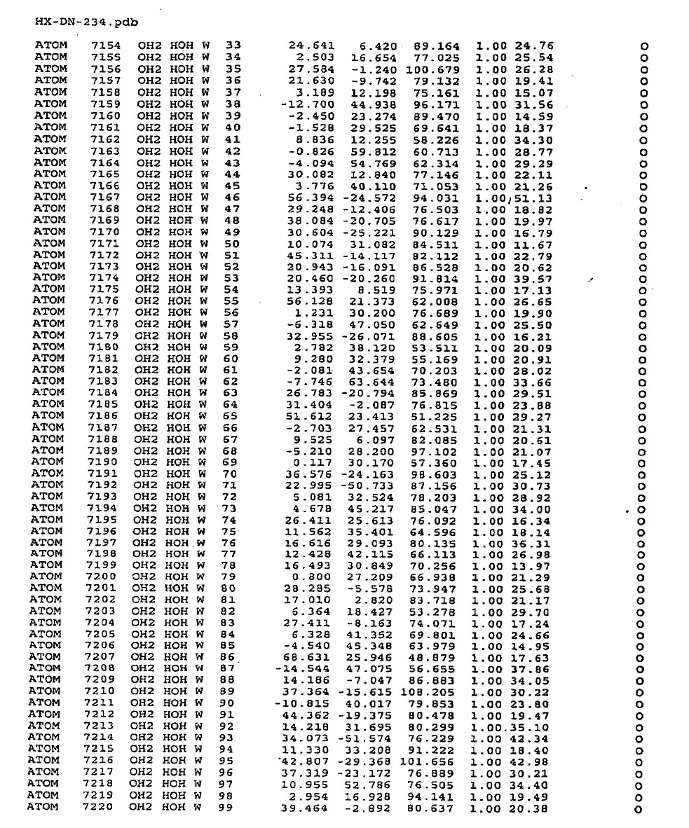

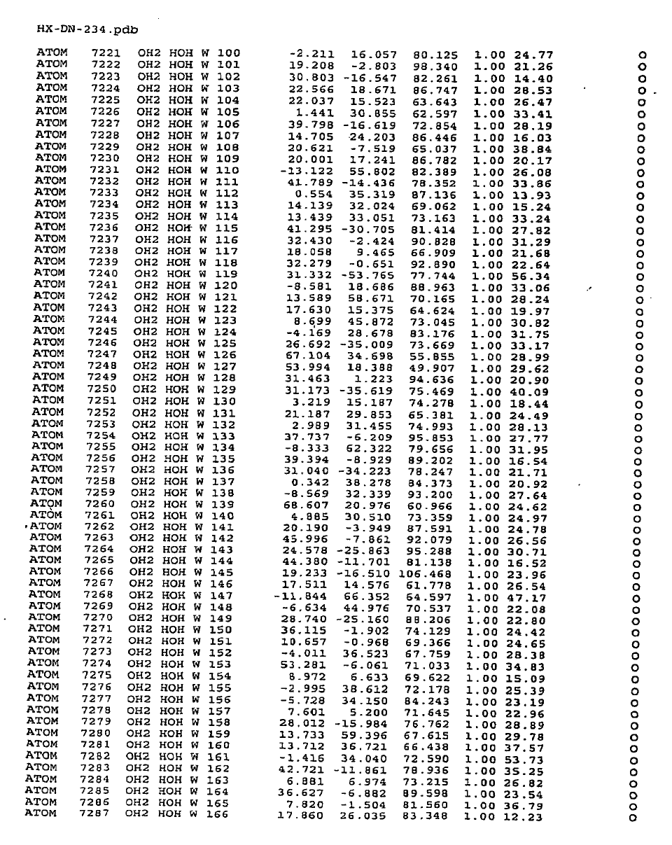

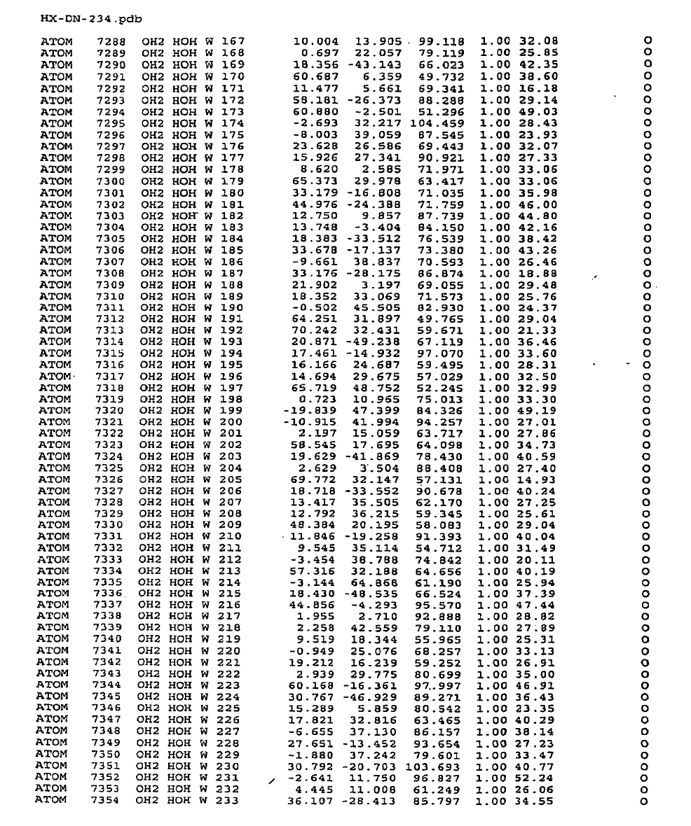

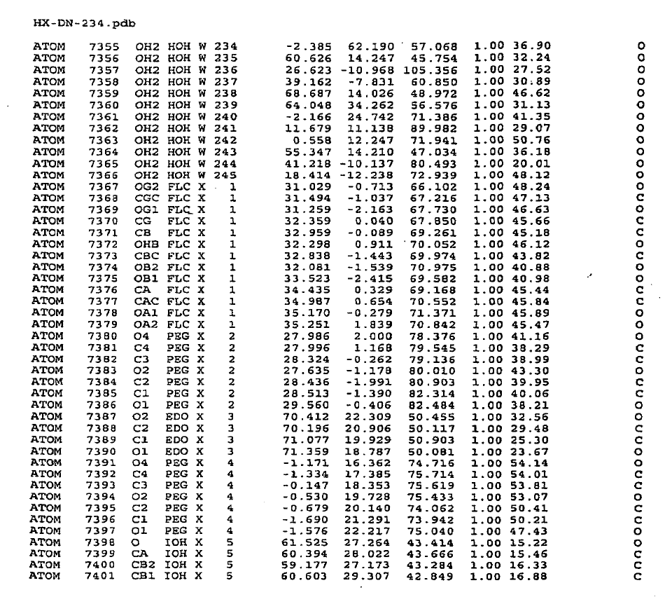

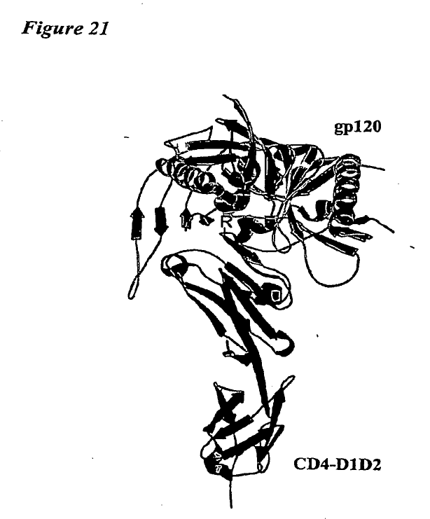

This figure shows Fₒ-F_{c} electron densities (2.5σ, blue) of modified Cys43 in comparison with the Phe43 cavity surface (red) in HX-WT complex. The structures of differently modified Cys43 from CD4 for all four HX-compound complexes and the structure of Phe43 in HX-WT complex (PDB-ID: 1RZJ) are shown as sticks, whereas gp120 (gold) and CD4 (hot pink) are shown as ribbons. In the stick models, carbon, nitrogen, oxygen, and sulfur atoms are colored green, blue, red, and yellow respectively. The electron densities for the four HX-compound complexes are obtained from simulated-annealing (10K) omit maps calculated by removing all chemical entities linked to position 43 starting from sulfur of the cysteines. The orientations of all figures are the same as that in FIG. 21.

FIGS. 23A & 23B

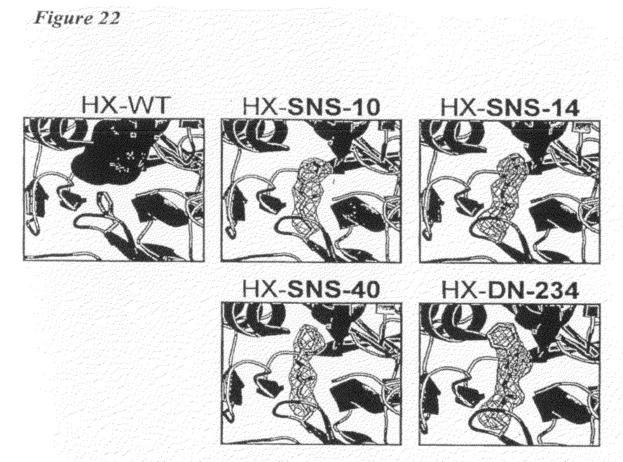

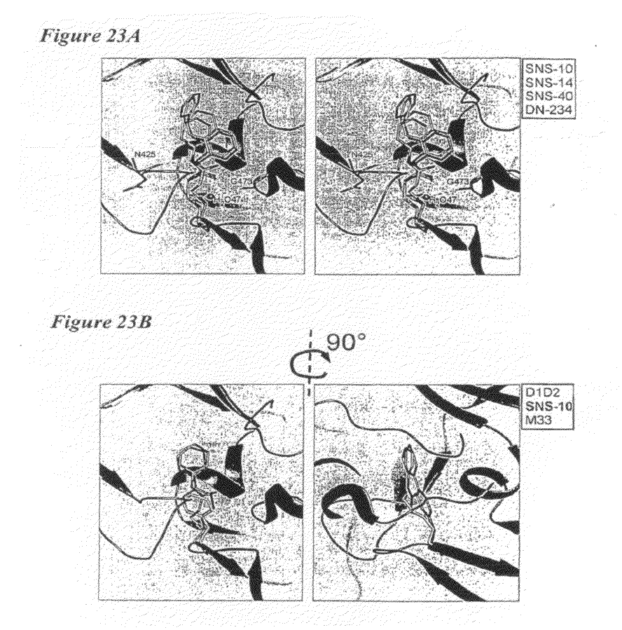

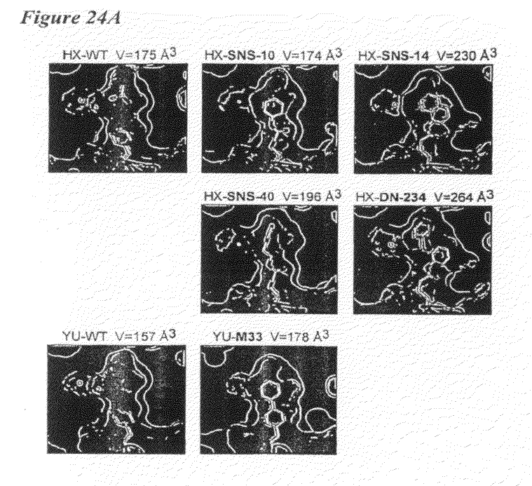

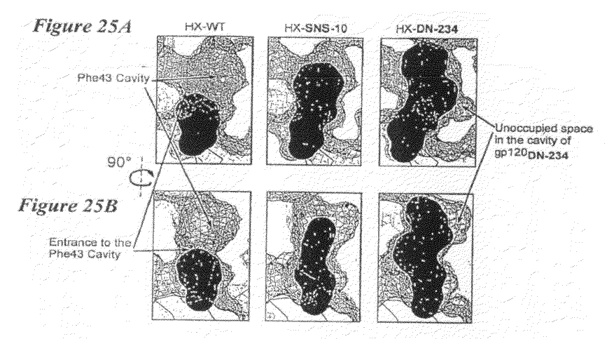

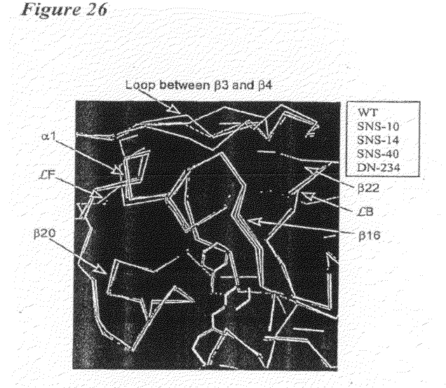

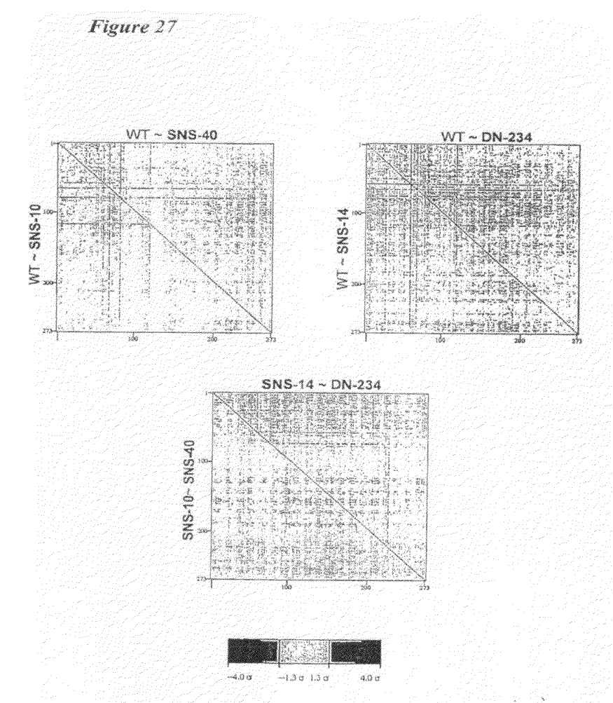

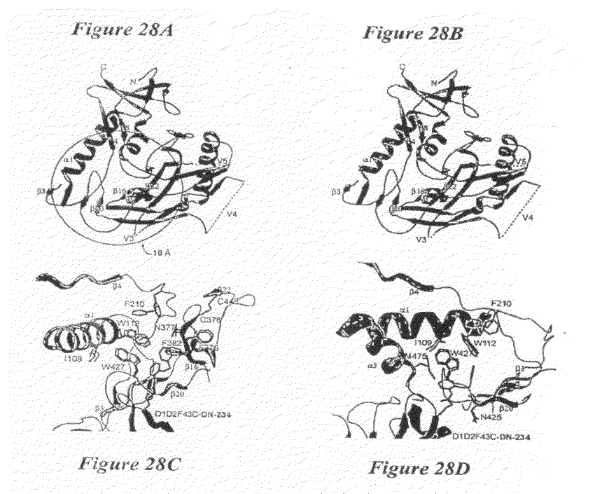

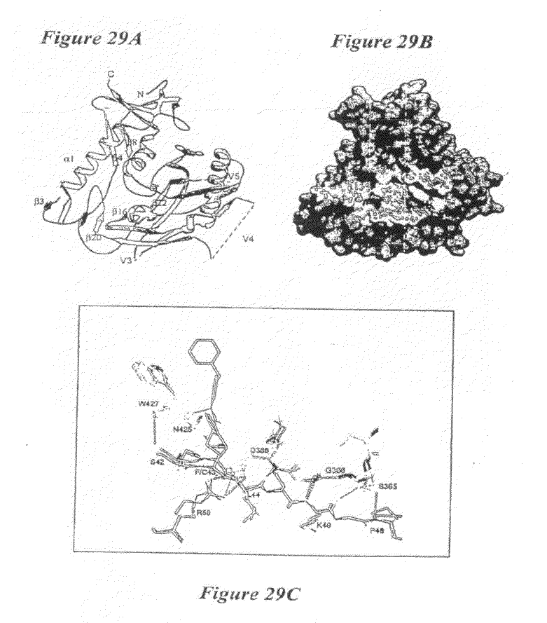

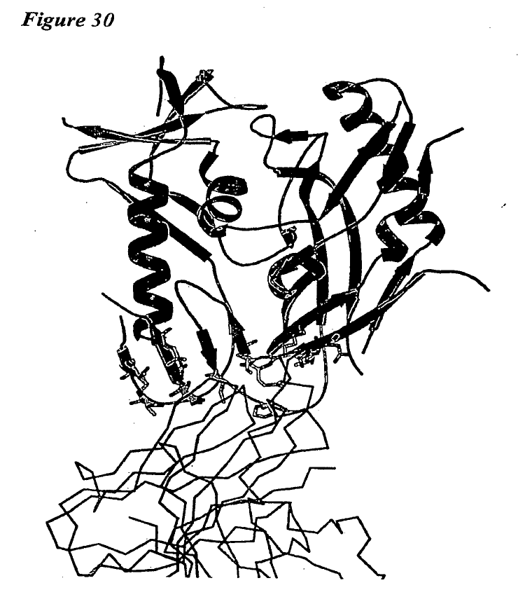

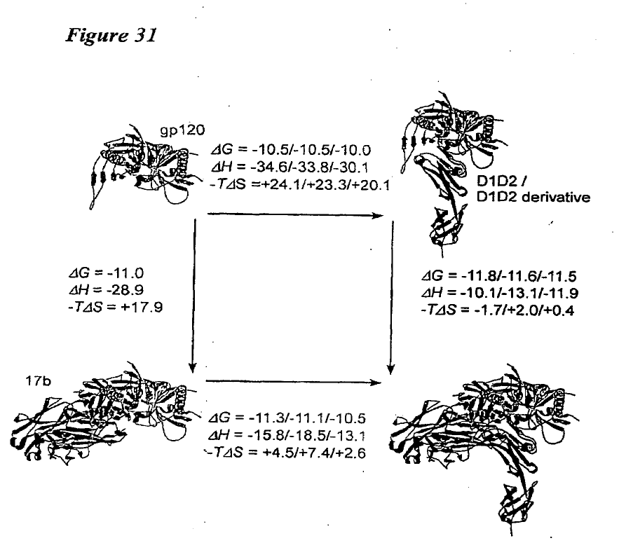

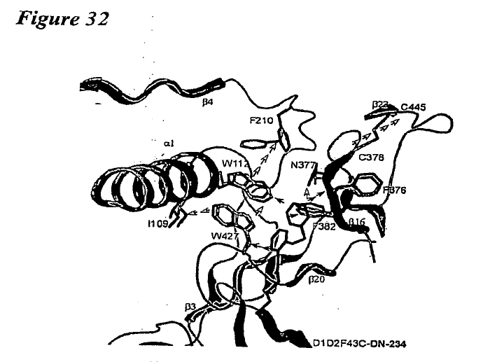

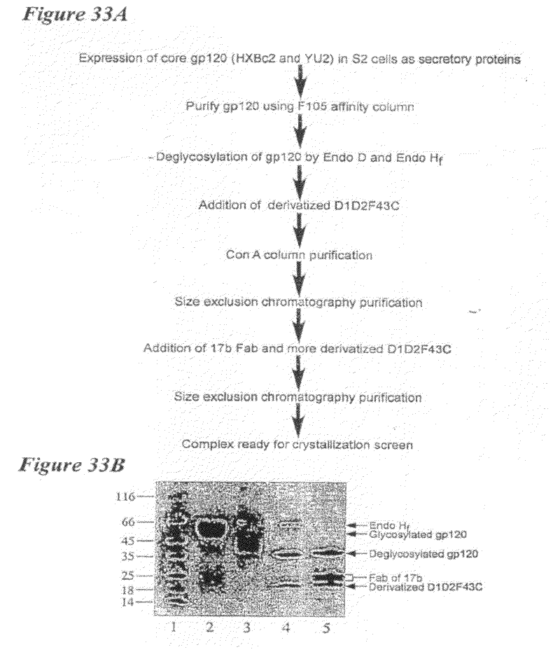

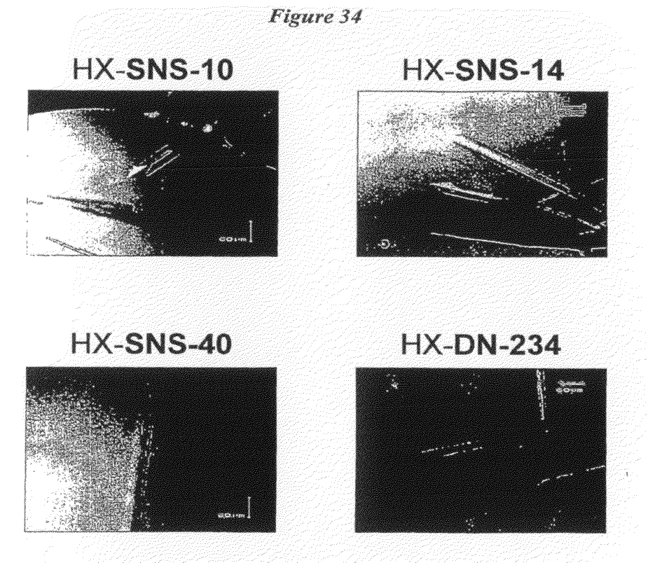

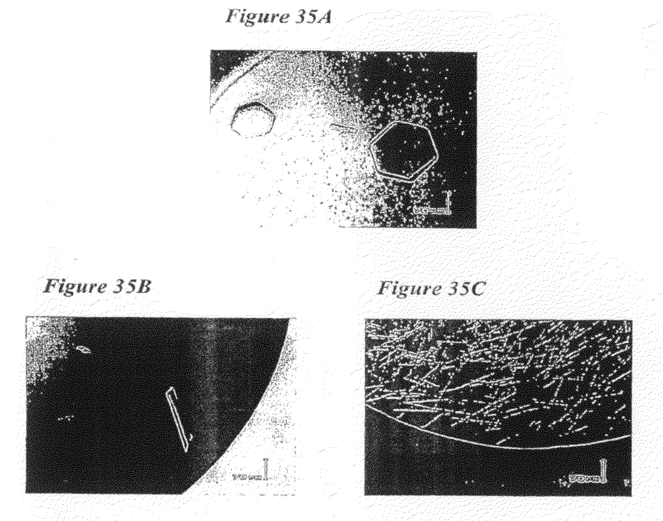

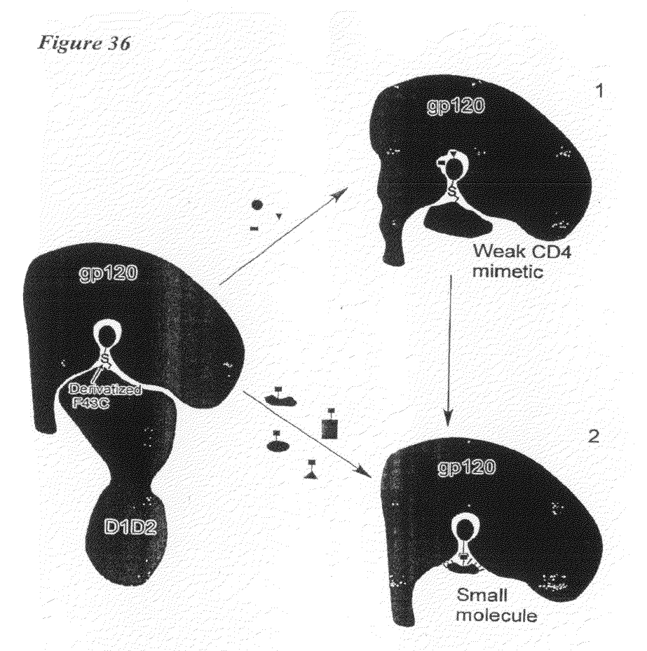

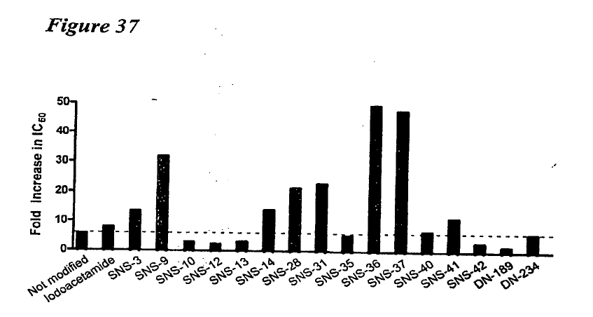

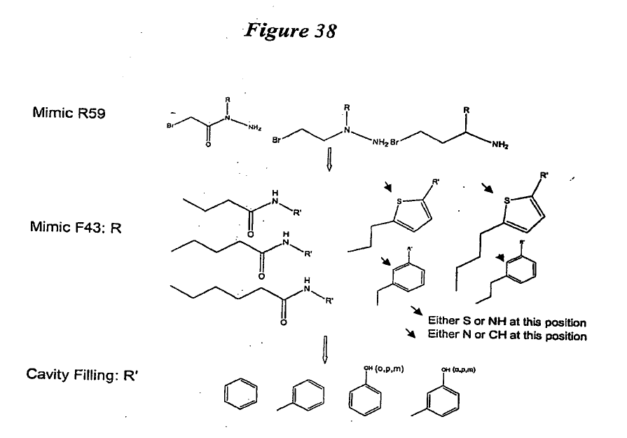

These figures show two different binding modes for interaction of CD4 derivatives and CD4M33 with Phe43 cavity. FIG. 23A: Stereoplot of all four D1D2F43C linked compounds shown as stick models in the Phe43 cavity. Side chains of Asn425 of gp120 in HX-SNS-10 complex and Gly473 of gp120 in HX-DN-234 complex are also shown as stick models. Nitrogen, oxygen, and sulfur atoms are colored blue, red, and yellow respectively, whereas the carbons for residues from HX-SNS-10, HX-SNS-14, HX-SNS-40 and HX-DN-234 are colored magenta, green, salmon and teal respectively. A water molecule (H₂O47) from HX-DN-234 complex is shown as a red sphere. Hydrogen bonds are depicted as dashed lines in the same color as the carbon atoms of the complexes where the hydrogen bonds occur. gp120 (gold) and CD4 (hot pink) from HX-10 complex are shown as ribbons. All four HX-compounds complexes have been superimposed onto HX-WT complex (Drawn in FIG. 23B.) using Cα of all residues in the gp120 invariant region (See text). The orientation is related to FIG. 22 by an 80° rotation about a vertical axis. FIG. 23B: Superimpositions of HX-WT (PDB-ID: 1RZJ), HX-SNS-10 and YU-M33 (PDB-ID: 1YYL) and around the cavity region using Cα of all residues in the gp120 invariant region (See text). Phe43 from HX-WT, cysteine linked SNS-10 from HX-SNS-10 and bi-phenyl group from YU-M33 are drawn as stick models in which the carbon atoms are colored grey, magenta and blue respectively, whereas nitrogen, oxygen, and sulfur atoms are colored blue, red, and yellow individually. gp120 (gold) and CD4 (hot pink) from HX-10 complex are also shown as ribbons. The orientation of the left panel is the same as that in FIG. 23A), whereas the orientation of the right panel is related to the left panel by a 90° rotation about a vertical axis.

FIGS. 24A-24C

These figures show the extensive interactions between the Phe43 cavity and the derivatized D1D2, enlarged Phe43 cavity and expanded water channel in gp120 complexed with D1D2 derivatives. FIG. 24A: Sliced-open surface representations and the volumes of the Phe43 cavities of gp120 in different complexes. The surface of gp120 extracted out of each complex is colored black for its interior side and cyan for its outside except for the CD4-interaction residues. The surfaces of the gp120 residues that interact with side chain of F43 in the HX-WT (PDB-ID: 1RZJ) or YU-WT (PDB-ID: 1G9N) complexes are colored green in all 5 HX complexes or 2 YU complexes respectively. Additional gp120 residues that interact with different cavity-filling entities are colored red. All surfaces are sliced open for better view of the cavity-filling entities as well as the locations of the CD4-interacting residues on the surfaces. The volume for the Phe43 cavity was calculated by the MS program (Connolly 1993) using a 1.4 Å probe and is listed for gp120 in each complex. D1D2 or D1D2 mimetic CD4M33 (PDB-ID: 1YYL) from each complex is shown as ribbons colored in hot pink. The chemical entities extended out from the position 43 of D1D2 (or equivalent position 33 in YU-M33 complex) are shown as sticks, in which carbon, nitrogen, oxygen, and sulfur atoms are colored grey, blue, red, and yellow respectively. In HX-WT complexes, the isopropanol molecule identified in the cavity is also shown as sticks using the same color scheme above. Water molecules located in the water channel adjacent to the cavity in each complex are depicted as yellow spheres. The orientations of all complexes are the same, and are similar to that in FIG. 23A. For a clear view of bi-phenyl group in the cavity, the clip plane for YU-M33 complex is slightly different than for the rest of the complexes. FIG. 24B: Linear correlation between the size of the Phe43 cavity and the molecule weight (M.W.) of the chemical entity residing in the cavity of gp120 bound by the derivatized D1D2. Based on the structures, the chemical groups attached to the acetamide nitrogen atom are regarded as the parts of the derivatives occupying the cavity and were used for calculation of the corresponding molecular weight (M.W.). FIG. 24C: Sliced-open surface representations of both the Phe43 cavity and the water channel in gp120 of HX-WT and HX-SNS-10 complexes. The interior and outside surfaces are colored dark purple and green respectively. D1D2 from each complex is shown as ribbons in hot pink. F43 of D1D2, isopropanol molecule from HX-WT and F43C-SNS-10 from HX-SNS-10 are draws as sticks using color scheme similar to that in FIG. 24A, except the color of the carbon atoms is yellow. Water molecules in the water channels are shown as red spheres. The orientation in FIG. 24C is related to that in FIG. 24A by a 45′ rotation about a vertical axis.

FIGS. 25A & 25B

These figures show the surface complementarity between the Phe43 cavity in gp120 and derivatized CD4 from different HX complexes. For clarity, the molecular surface of gp120 are shown in both transparent and mesh representations in cyan. The side chains of (derivatized) residue 43 are depicted as solid surfaces in magenta. The Cα traces of D1D2/D1D2 derivatives are also in magenta. The viewing angle of FIG. 25A is the same as FIG. 23A. The models in FIG. 25B are rotated clockwise by 90° around a vertical axis in the plane of the page.

FIG. 26

This figure shows the superimpositions of Cα traces of gp120 bound to D1D2 or its derivatives. Only gp120 regions close to the Phe43 cavity are shown and they are colored in white, blue, orange, green, and pink for gp120_{D1D2}, gp120_{SNS-10}, gp120_{SNS-14}, gp120_{SNS-40}, and gp120_{DN-234}. The superimpositions are based on the Cα atoms of invariant regions of gp120 identified by ESCET. For simplicity, only the side chain of modified Cys43 of D1D2F43C-DN-234 is shown in sticks (magenta).

FIG. 27

This figure shows distance-sorted error-scaled difference-distance matrices for selected pairs of different gp120 structures extracted from their complexes with D1D2 or its derivatives. WT, SNS-10, SNS-14, SNS-40, and DN-234 stand for gp120_{D1D2}, gp120_{SNS-10}, gp120_{SNS-14}, gp120_{SNS-40}, and gp120_{DN-234} respectively. gp120 residues were first sorted in an ascending order by the distances between their Cα atoms and the center of Phe43 cavity (defined by the C4 atom of the phenyl ring of residue 43 in D1D2F43C-SNS-10). The Cα atoms of all 273 residues (85-126, 196-297, 330-392, 415-459, and 471-491), which do not belong to the variable regions V1-V5 (Modrow et al. 1987; Leonard et al. 1990), were used for calculation of error-scaled difference-distance matrices. Because these matrices are symmetrical, only half of them (either the upper right or the low left half) are shown. For each matrix between a pair of gp120 structure “a” and “b”, the matrix element Eᵢⱼ^{ab} was calculated by the equation Eᵢⱼ^{ab}=Δᵢⱼ^{ab}/σ(Δᵢⱼ^{ab})=(|rᵢ^{a}−rⱼ^{b}|)/σ(Δᵢⱼ^{ab}), where Δᵢⱼ^{ab} stands for the difference distance of a pair of atom i and j between model “a” and “b”; rᵢ^{a} denotes the Cartesian coordinate vector of atom i in model “a”; and σ(Δᵢⱼ^{ab}) is the estimated standard derivation for the matrix elements derived from the quality of the diffraction data and atomic B factors (Schneider 2000). Matrix elements are colored according to the bar at the bottom of figure: elements with absolute value less than 1.3σ(Δᵢⱼ^{ab}) are colored grey; elements between 1.3σ(Δᵢⱼ^{ab}) and 4σ(Δᵢⱼ^{ab}) are colored by the color gradients—blue for negative changes (expansion of distance between atom i and j in model “b” with respect to “a”) and red for positive changes (contraction); elements larger than 4Δᵢⱼ^{ab} (negative or position) are shown as full blue or red respectively.

FIGS. 28A-28D

These figures show the flexible regions in gp120. FIG. 28A: Flexible regions of gp120_{DN-234} identified by ESCET. With the backbone in ribbon representation, the flexible regions (106-117, 209-213, 249-253, 376-377, 410-41.1, 421-430, and 444-445) are colored in red; the other segments are in blue. A cross-section of a 18 Å sphere around the Phe43 cavity is drawn as a dark red circle. The orientation of gp120 is a 90° rotation of the viewing angle shown in FIG. 21, around a horizontal axis. FIG. 28B is in the same orientation as FIG. 28A. The flexible gp120 residues (red in FIG. 28A) that interact or do not interact directly with CD4F43C-DN-234 are colored hot pink or orange respectively, whereas CD4F43C-DN-234 interacting residues of gp120 that do not belong to flexible regions are in green; the other segments are in blue. FIG. 28C: gp120_{DN-234} (cyan) and CD4F43C-DN-234 (dark grey) are drawn as ribbons in an orientation 45° rotation about a vertical axis to that in FIG. 21. The flexible gp120 residues that interact or do not interact with CD4F43C-DN-234 are colored hot pink or orange respectively, as in FIG. 28B. Side chains of selected flexible gp120 residues for both gp120_{DN-234} (red) and gp120_{D1D2} (green) as well as the modification on Cys43 of CD4F43C-DN-234 (dark grey) are shown as stick model. The disulfide bond between Cys445 and Cys378 is shown as yellow stick connecting Cα and sulfur atoms in two residues. FIG. 28D: A 60° rotation view of FIG. 28C about a vertical axis. β16 is removed for better view of residue M475. The coloring scheme is the same as in FIG. 28C.

FIGS. 29A-29C

This figure shows the gp120-CD4 interface in HX-DN-234 complex. FIG. 29A: Mapping of CD4 interacting residues on ribbon representation of gp120_{DN-234} (blue). gp120 residues that interact with both wild type D1D2 and D1D2F43C-DN-234 are colored green and gp120 residues that only interact with D1D2F43C-DN-234 are colored red. The orientation is same as that in FIG. 28A. FIG. 29B: Surface representation of FIG. 29A with same color scheme. FIG. 29C: Comparison of hydrogen bonding interactions between CD4 and gp120 in HX-WT and HX-DN-234 complexes. The interfacial hydrogen bonds shorter than 3.5 Å in HX-WT and HX-DN-234 complexes are drawn as yellow or black dashed lines respectively. C″ strand (residue 42-28) and residue 59 of CD4 in both complexes are shown as thin sticks, whereas residue 425, 427, and β15 strand (365-368) of gp120 are drawn as thick sticks. Carbon atoms are colored either green (HX-WT complex) or pink (HX-DN-234 complex). Oxygen, nitrogen and sulfur atoms are colored red, blue, and yellow respectively.

FIG. 30

This figure shows the gp120-17b interface. gp120 is shown as ribbon and colored similarly as in FIG. 28B except the base color for gp120 is grey instead of cyan. The Cα trace of 17b is colored in blue. All 17b-interacting gp120 residues are displayed as stick models with carbon, nitrogen, oxygen and sulfur atoms colored cyan, blue, red, and yellow respectively.

FIG. 31

This figure shows the thermodynamic cycles of binding of 17b and D1D2 (black)/D1D2F43C-SNS-10 (blue)/D1D2F43C-DN-234 (red) to YU2 gp120.

FIG. 32

This figure shows the pathway for motion propagation of gp120 residues in binding D1D2F43C-DN-234. Selected gp120 residues that display plasticity in binding D1D2F43C-DN-234 are same as shown in FIG. 28C. Filled black arrow denotes the interactions between gp120 and DN-234, which lead to the structural rearrangement in the corresponding gp120 residues. Open black arrow represents the inter-atomic contacts between gp120 residues, which are responsible for the secondary motions propagated from gp120 residues that directly interact with DN-234.

FIGS. 33A & 33B

These figures show the preparation of ternary complex of gp120 with D1D2 derivatives. FIG. 33A: The flow chart of the preparation process of the complex; FIG. 33B: SDS-PAGE analysis of HXBc2 gp120 and its complex during the process of the complex formation. Lane 1: molecular weight markers; Lane 2: gp120; Lane 3: gp120 partially deglycosylated by being treated with Endo H_{f}; Lane 4: gp120 treated with Endo H_{f} and Endo D derivatized D1D2F43C was also added to stabilize gp120; Lane 5: the final ternary complex.

FIG. 34

This figure shows the crystals of four ternary complexes composed of HXBc2 gp120, 17b and different derivatized D1D2. HXBc2 gp120:17b:CD4-derivative complexes are abbreviated as HX-compound correspondingly.

FIGS. 35A-35C

These figures show the crystals of two ternary complexes composed of YU2 gp120, 17b and D1D2 or derivatized D1D2. FIG. 35A: A hexagonal crystal of YU-WT (YU2 gp120:17b Fab:D1D2) crystallized from similar condition for original YU-WT complex (Kwong et al. 2000). FIGS. 35B and 35C show two different crystals of YU-SNS-10 (YU2 gp120:17b Fab:D1D2F43C-SNS-10). The crystallization conditions are as following: FIG. 35B: 10% PEG 1K and 0.05 M Tris, pH 7; FIG. 35C: 0.1M calcium acetate, 9-10% PEG 8K and 0.05 M Na Cacodylate, pH 6.5.

FIG. 36

This figure shows future directions for the design of gp120-CD4 antagonist. Two possible directions are depicted starting from the identified cavity-targeting chemical modules: 1) further optimization of the cavity-binding ligands by using a weak CD4 mimetics; 2) screening and assembly of small molecules that recognize not only the Phe43 cavity but also the vestibule to the cavity and Arg59 site.

FIG. 37

This figure shows the ratio of IC₅₀ of D1D2F43C:R59A derivatives to gp120:D1D2 binding compared with that of corresponding D1D2F43C derivatives modified from same compounds. The compound name for deriving both derivatives in IC₅₀ comparison is listed under corresponding column. A dash line parallel to X-axis is shown with a Y-axis intersection of 5.6, the value for the ratio of IC₅₀ of D1D2F43C:R59A to D1D2F43C.

FIG. 38

This figure shows the fragments proposed for the assembly of cysteine-modification compounds for D1D2F43A:R59C scaffold.

### DETAILED DESCRIPTION OF THE INVENTION

### Embodiments of the Invention

This invention provides soluble CD4-based polypeptides and compositions comprising same. The first soluble polypeptide consists of a portion of CD4 comprising all HIV gp120-binding epitopes present on intact CD4, wherein the polypeptide has a cysteine substitution at a residue which, in intact CD4, interfaces with HIV gp120.

The second soluble polypeptide comprises (i) a portion of CD4 comprising all HIV gp120-binding epitopes present on intact CD4, wherein the polypeptide has a cysteine substitution at a residue which, in intact CD4, interfaces with HIV gp120, and (ii) a chemical moiety bound to the CD4 portion at the cysteine substitution via a thiol bond.

The third soluble polypeptide comprises intact CD4, wherein the intact soluble CD4 has a cysteine substitution at a residue which interfaces with HIV gp120.

The fourth soluble polypeptide comprises (i) intact CD4, wherein the soluble CD4 has a cysteine substitution at a residue which interfaces with HIV gp120, and (ii) a chemical moiety bound to the intact soluble CD4 at the cysteine substitution via a thiol bond.

The fifth soluble polypeptide comprises (i) intact soluble CD4 or a portion of intact soluble CD4 covalently bound to (ii) a polypeptide moiety (e.g. an Ig polypeptide), wherein the intact soluble CD4 or portion thereof has a cysteine substitution at a residue which interfaces with HIV gp120.

The sixth soluble polypeptide comprises (i) intact soluble CD4 or a portion of intact soluble CD4 covalently bound to (ii) a polypeptide moiety (e.g. an Ig polypeptide), wherein the intact soluble CD4 or portion thereof has a cysteine substitution at a residue which interfaces with HIV gp120 and (iii) a chemical moiety bound to the intact soluble CD4 at the cysteine substitution via a thiol bond.

Herein, the first through sixth soluble polypeptides are referred to individually and collectively as CD4-based polypeptides.

In one embodiment, the portion of CD4 is the portion designated D1D2. In a second embodiment, the cysteine substitution is an F43C or R59C substitution. In another embodiment, the HIV gp120 is HIV-1 gp120.

In a further embodiment, the chemical moiety is bound to the intact soluble CD4 or CD4 portion via reaction with a haloacetamide, a halopropanone or a 5-nitro-2-pyridinesulfenyl reagent. In another embodiment, the chemical moiety is bound to the intact soluble CD4 or CD4 portion via reaction with 2-Bromo-N-(4-nitro-phenyl)-acetamide. In another embodiment, the polypeptide (e.g. second, fourth or sixth) binds to HIV gp120 with an IC₅₀ of ≦10 nM. In a final embodiment, the polypeptide (e.g. second, fourth or sixth) binds to HIV gp120 with an IC₅₀ of ≦5 nM.

This invention also provides two methods. The first is a method for making a derivatized soluble polypeptide comprising contacting, under suitable conditions, (a) a thiol-reactive reagent with (b) the first, third or fifth soluble polypeptide, wherein the polypeptide has a cysteine substitution at a residue which, in intact CD4, interfaces with HIV gp120.

The second is a method for obtaining a structural model useful in the design of an agent for inhibiting CD4 binding to HIV gp120 comprising (a) identifying a second, fourth or sixth soluble polypeptide which binds to HIV gp120 with an affinity comparable to or greater than the affinity with which intact CD4 binds to HIV gp120; and (b) obtaining a three-dimensional structure of the identified polypeptide while it is bound to HIV gp120, thereby obtaining a structural model useful in the design of an agent for inhibiting CD4 binding to HIV gp120.

In one embodiment of both methods, the portion of CD4 is the portion designated D1D2. In a second embodiment, the cysteine substitution is an F43C or R59C substitution. In another embodiment, the HIV gp120 is HIV-1 gp120.

In a further embodiment of the first method, the thiol reactive agent is a haloacetamide, a halopropanone or a 5-nitro-2-pyridinesulfenyl reagent.

In a further embodiment of the second method, the chemical moiety of the polypeptide is bound to the CD4-based polypeptide via reaction with a haloacetamide, a halopropanone or a 5-nitro-2-pyridinesulfenyl reagent. In another embodiment of the second method, the polypeptide binds to HIV gp120 with an IC₅₀ of ≦10 nM. In a final embodiment of the second method, the polypeptide binds to HIV gp120 with an IC₅₀ of ≦5 nM.

This invention further provides methods for identifying and for designing candidate inhibitors of CD4/gp120 binding comprising identifying desired chemical features for such compounds based on the structural information herein regarding the gp120/CD4 interface (e.g. Figures, Example IV and Example V).

This instant invention is illustrated in the Experimental Details section that follows. This section is set forth to aid in an understanding of the instant invention but is not intended to, and should not be construed to, limit in any way the invention as set forth in the claims which follow thereafter.

### EXPERIMENTAL DETAILS

### Example I

### Synopsis of Features of the Invention

Crystal structures of complexes between HIV gp120 envelope glycoproteins and the cellular receptor CD4 defined their high-affinity (nM level) interaction at an atomic level. This includes a cavity in the interface near CD4 residue phenylalanine 43 (Phe43) at the center of the interface. Although HIV proteins mutate readily to escape the immune system, determinants of the unique and specific interaction with human CD4 are preserved. Subsequent thermodynamic and spectroscopic studies showed that large conformational changes occur in gp120 upon CD4 binding, suggesting that epitopes for CD4 binding are hidden from the immune system in apo gp120. It would be desirable to develop inhibitors that compete effectively with HIV sites for CD4 binding, but the exceptional flexibility of gp120 complicates lead identification. High-throughput screens have had little success in this system.

We have devised a method for identifying chemical leads for inhibition of the gp120-CD4 interaction. We use a D1D2 construct of CD4 that includes all of the gp120 binding epitopes, which we mutate to introduce a site for chemical derivatization. Most of the work has been done with the F43C variant (CD4 Phe43 Cys43), but other variants including R59C have also been used. CD4 thus mutated is reacted with chemicals, such as bromoacetamides or 5-nitro-2-pyridinesulfenyl reagents, that will react with the free thiol that has been introduced. Binding affinity of the derivatized CD4 (D1D2) is tested in an ELISA assay for binding to full-length gp120 molecules. F43C CD4 is impaired in gp120 affinity relative to wild type, but high affinity is restored with certain of the derivatives. Complexes of chemically derivatized CD4 with core gp120 molecules can be isolated and purified, and four of these have been crystallized and subjected to structure analysis by x-ray crystallography. Since the chemical modification is buried into the interface between two proteins, the exterior surface remains the same and these complexes crystallize isomorphously with the wild type structures. The structures show in detail how the chemical additions bind into the F43 cavity, and they motivate the design of new chemical derivatives aimed at higher potency and the exploration of a water channel beyond the water cavity.

The CD4 derivatives identified by this method will serve as leads for further development. Ultimately they will be attached to other, non-CD4 scaffolds for further elaboration. An intermediate step will be to use smaller peptide mimetics of CD4 that also contain a cysteine residue for derivatization. Ultimately, we expect to keep the chemical portions found to bind optimally into the Phe 43 cavity and to elaborate chemical replacements for all of the CD4 protein. Such compounds will be legitimate leads for drug development as HIV entry inhibitors.

### Example II

### Structure-Activity Relationships in the Binding of Chemically Derivatized CD4 to HIV gp120

### Synopsis

Recognition of the HIV envelope protein gp120 by the host cell receptor CD4 is the first step in HIV infection. An interfacial “Phe43 cavity” in gp120, close to where the CD4 residue Phe43 is bound, has been suggested as a potential target for therapeutic intervention. Because this cavity is unique to CD4-bound gp120, we designed and prepared a two domain CD4 template with Phe43 mutated to the chemoselective cysteine residue for site-specific coupling of chemically diverse compounds for screening against the Phe43 cavity. A library of haloacetamides and 5-nitro-2-pyridyldisulfides were selected and synthesized for modification of the reactive cysteine on CD4. Among them, 2-Bromo-N-(4-nitro-phenyl)-acetamide (compound DN-052) produced a CD4 derivative with highest affinity in binding gp120 (IC₅₀=4.14 nM). The structure-activity relationship (SAR) study of derivatized CD4 binding to gp120 revealed a significant plasticity of the Phe43 cavity in binding different compounds and a narrow entrance to the cavity. The primary contacts for compound recognition by the cavity were found to be van der Waals interactions, while hydrophilic interactions were detected at the narrow entrance region. This first SAR on ligand binding to an interior cavity of gp120 may provide a starting point for structure-based assembly of small molecules targeting gp120-CD4 interaction.

### Introduction

A novel method of identifying low-affinity cavity binder needs to be developed. Also, special consideration is needed to ensure the stabilization of Phe43 cavity, which is absent in free gp120 (Kwong, Wyatt et al. 1998; Kwong, Wyatt et al. 2000; Chen, Vogan et al. 2005). Once identified, the low-affinity small molecules that targets Phe43 cavity could be used in combination with other fragments that recognize Phe43 site (the entrance of the pocket) or Arg59 site on gp120 by fragment-assembly approaches (Rees, Congreve et al. 2004).

We addressed this question using a protein-small molecule hybrid approach. The protein template in the hybrid retained enough activity to structure Phe43 cavity in gp120, while providing chemically active site for site-specific coupling of chemically diverse compounds for screening against the Phe43 cavity (Smith, Savinov et al. 2002). Here, we report the construction of 81 protein-small molecule hybrids derived from a library of various small compounds, which would otherwise bind weakly to gp120 without the template, and the first structure-activity relationships for the interactions of Phe43 cavity targeting probes with gp120.

### Experimental Design

### Reagents

Iodoacetamide, 5,5′-Dithiobis(2-nitrobenzoic acid) (DTNB) and N-Ethylmaleimide (NEM) were purchased from Sigma-Aldrich. All other cysteine-modification compounds were synthesized as described in Chemistry section below. The gp120 antibody 17b was produced in ascites and purified by Strategic BioSolutions. Purified full length YU2 gp120 from S2 cells was provided by Dr. R. Wyatt. Purified full length YU2 S375W/T257S and wild type YU2 produced in HEK293 cells were also obtained from Dr. R. Wyatt.

### Preparation of Recombinant D1D2 and D1D2 Mutants

Recombinant two domain CD4 (D1D2, residue 1-183) was cloned into NcoI and XhoI sites of vector pET24d (Novagen). D1D2 was expressed as inclusion bodies in Rosetta (DE3) cells (Novagen) through leaky-expression of T7lac promoter without IPTG induction in SuperBroth medium (BIO 101, Inc.) at 37° C. for 24 hours. The inclusion bodies were isolated from cells by sonication and centrifugation, and then washed three time by 2% Triton-X 100 (Sigma-Aldrich), 2 M urea (Fisher Scientific), 5 mM EDTA and 5 mM DTT in Tris.HCl buffer, pH 7.5. Pure inclusion bodies were further solubilized by 6 M guanidine-HCl, 5 mM EDTA, 20 mM Tris.HCl, pH 7.5, 10 mM DTT, 0.5 mM PMSF. Solubilized D1D2 were refolded at 0.5 mg/ml in a refolding solution optimized based on the #10 condition of Foldit kit (Hampton Research): 50 mM Tris.HCl, pH 8, 10 mM NaCl, 1 mM KCl, 1 mM EDTA, 440 mM Sucrose, 2 mM Cysteine and 2 mM cystine. The impurity and oligomeric D1D2 were removed by passing refolded D1D2 proteins through Q and SP Sepharose Fast Flow resins (Amersham) in batch-mode at pH 10.5 and pH 6.2 respectively. As a last step, a size exclusion column Superdex 200 26/60 (Amersham) was used to separate any residual misfolded oligomeric D1D2 from monomeric D1D2. Purified soluble D1D2 contains residue 1-183 of CD4 and an additional glycine from the cloning vector at the N-terminus. CD4 mutants D1D2F43C and D1D2F43Y were created by site-directed mutagenesis and prepared similarly as wild type D1D2.

### Preparation of Derivatized D1D2F43C

Iodoacetamide, DTNB and NEM were dissolved and store as 20 mM solution in 0.5 M Na/K-phosphate, pH 7.4. All other compounds including both bromo-compounds and mix-disulfide compounds were dissolved in DMF, DMSO or ethanol as 20-60 mM stock. D1D2F43C proteins at 1-3 mg/ml were first reduced by 2 mM Dithiothreitol (DTT) (BioVectra). Excessive DTT was then removed using PD-10 columns (Amersham) and at the same time the proteins were exchanged into proper reaction buffer: 0.1 M phosphate buffer, pH 7.4, 0.1 M NaCl, 1 mM EDTA and 0.1 mM DTT for all Bromo-compounds, Iodoacetamide and DTNB; 0.1 M phosphate buffer, pH 7.0, 0.1 M NaCl, 1 mM EDTA for 5-nitro-2-pyridinesulfenyl compounds and 0.1 M phosphate buffer, pH 7.4, 0.1 M NaCl, 1 mM EDTA and 0.1 mM DTT for NEM. The thiol-reactive compounds were then diluted to protein solutions to reach final concentration of 2 mM, which is more than 10 folds of the free thiols's concentration in reaction. The reactions were allowed to continue at 25° C. for 2 hours in the dark. The final products, namely derivatized D1D2F43C, were separated from small molecules through desalting columns (PD-10 columns, Amersham) and solvent exchange in Amicon Ultra-4 5K concentrator (Millipore). The completeness of the modifications was examined using protein mass spectrometry (MALDI-TOF) as well as peptide mass spectrometry of trypsin-digested proteins. This latter step was also used to confirm the correct site-directed incorporation of the compounds. A control D1D2 was prepared by using compound 40 to “mock-modify” D1D2 in the same way described above. The product of this reaction was thus name D1D2-40.

### Concentration Determination of Derivatized D1D2F43C Proteins

Two independent methods were used. A) Amino acid analysis (Keck Biotechnology Resource Laboratory, Yale University) was used for determinations of the concentrations of a selected group of D1D2F43C derivatives. Only the results of residue Ala, Leu and Phe were used in the calculation of protein concentrations. B) The “nominal concentrations” of proteins were calculated from 280 nm absorbance of protein samples in PBS using theoretic extinction coefficient of D1D2F43C. The concentrations of D1D2F43C variants derived from the halo-compounds were then corrected by the corresponding correction factors: Correction factor (%) OD280_{D1D2F43C}/(OD280_{D1D2F43C}+OD280_{protein-conjugated compound}). OD280_{protein-conjugated} compound was estimated, by the experimentally determined absorbance of the free halo-compounds in PBS.

The concentrations of selected derivatives determined by method A were in good agreement with the results obtained by method B. Thus method B was then used for concentration determinations of all halocompound-derivatized proteins. The “nominal concentration” was used for derivatized D1D3F43C from all other compounds.

### Competitive ELISA Assay

The abilities of derivatized D1D2F43C proteins to bind gp120 and inhibit gp120-CD4 interaction were evaluated using a competition ELISA (Enzyme-Linked Immunosorbent Assay). Briefly, Immuno 2HB plates (Thermo LabSystem) were coated by with 100 μl 4 μg/ml recombinant D1D2 in PBS overnight at 4° C. The plates were then blocked by 3% bovine serum albumin (BSA) (CalBiochem) in PBS for 2 hrs at 25° C. Fifty nanogram YU2 gp120 from S2 cells, YU2 gp120 from HEK293 cells or YU2 S375W/T257S from HEK293 cells in total volume of 100 μl in 3% BSA-PBS was added to the plates in the presence of either D1D2 or D1D2F43C derivatives at various ranges of concentrations and incubated for 90 minutes at 25° C. After removal of unbound gp120 by washing plates four times with PBST (0.05% Tween-20 in PBS), the bound gp120 was detected by a gp120 antibody 17b (100 μl, 1 μg/ml), which was further probed with a peroxidase-conjugated donkey anti-human antibody (Jackson ImmunoResearch, 1:20,000 dilution, 100 μl). 3,3′,5,5′-tetramethylbenzidine (Sigma) was used as the substrate for peroxidase and the optical density (OD) was read at 450 nm. The binding of the residual gp120 to plate-bound D1D2 was calculated by using the following formula:

Binding (%)=100×(OD_{gp120-Competitor}−OD_{background})/(OD_{gp120}-OD_{background}).

IC₅₀ values were obtained by nonlinear regression fitting of binding results by GraphPad Prism 4 (GraphPad Software) using the formula of one site competition shown below:

Binding (%)=BOTTOM+(TOP−BOTTOM)/[1+10̂(log(Concentration)−log(IC₅₀))].

### Results

### Design Rationale

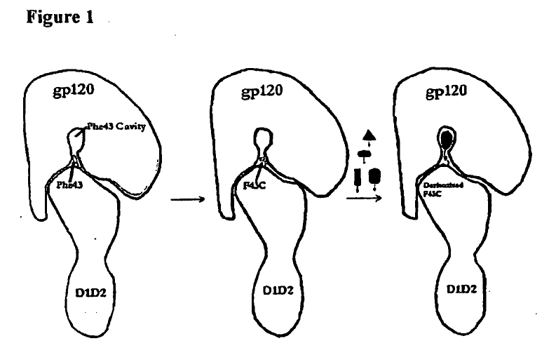

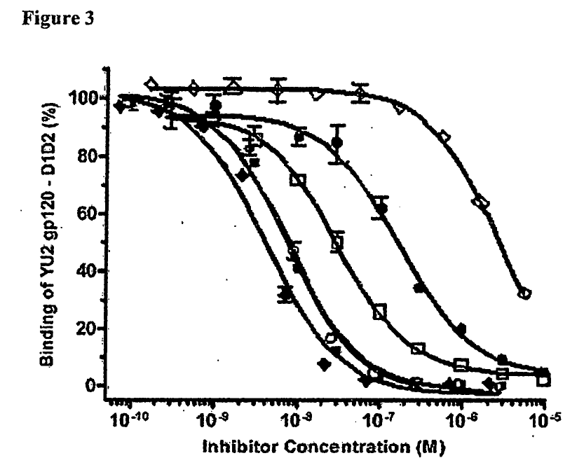

We used two-domain CD4 (D1D2) scaffold with Phe43 replaced by cysteine (D1D2F43C) as the template for site-specific delivery of diverse probes to the cavity. This template was chosen based on following considerations. First, D1D2 contains all the essential elements of CD4 in binding gp120. The D1D2's ability to co-crystallize with gp120 (Kwong, Wyatt et al. 1998; Kwong, Wyatt et al. 2000; Huang, Tang et al. 2005) opened a door for structural characterization of binding between gp120 and the derivatized CD4 proteins. Second, the exact location of residue Phe43 of CD4 at the entrance of the cavity made it the best position for attaching and specifically delivering chemical groups into cavity. The chemical modification of cysteine at position 43 would be straightforward given the high solvent accessibility of Phe43 revealed by the crystal structures of free CD4 (Ryu, Kwong et al. 1990; Wang, Yan et al. 1990; Ryu, Truneh et al. 1994; Wu, Kwong et al. 1997). Third, cysteine was chosen to replace Phe43 because it, among all natural amino acids, enables chemoselective conjugation best. Also all endogenous cysteines of D1D2 are disulfide-bonded (Ryu, Kwong et al. 1990; Wang, Yan et al. 1990; Ryu, Truneh et al. 1994; Wu, Kwong et al. 1997) and will not interfere with selective modification of F43C. Furthermore, D1D2F43C has much lower affinity to gp120 than D1D2 (FIG. 3), allowing the compatibility of a chemical group and the cavity to be easily judged by comparing the gp120-binding capabilities of the derivatized D1D2F43C protein to that of D1D2F43C (FIG. 1).

The binding affinities of the derivatized D1D2F43C to gp120 were evaluated by competitive ELISA assays, in which the potencies (IC₅₀) of these derivatives in inhibition of gp120-D1D2 binding were measured (FIG. 3). An IC₅₀ of 7.31 nM was obtained for a positive control D1D2-40 (prepared by “mock-modification” of D1D2 with compound 40) in this assay (FIG. 3). Because that D1D2F43C derivatives bind gp120 competitively with D1D2 and that the IC₅₀ value of D1D2 control agreed well with the reported Kd of D1D2 and gp120 (Smith, Byrn et al. 1987; Arthos, Deen et al. 1989), the measured IC₅₀ values of the CD4 derivatives could be regarded as the indirect measure of affinities between them and gp120.

In order to identify the best thiol-reacting module for linking diverse compounds to Cys43, one representative compound from each of three commonly-used classes of sulfhydryl reagents was tested for its performance in modification of F43C residue of D1D2 and the affinity of the derived D1D2F43C to gp120 was determined. The compounds, namely iodoacetamide, 5,5′-dithiobis(2-nitrobenzoic acid) (DTNB), and N-ethylmaleimide (NEM), all successfully modified D1D2F43C with satisfactory completeness (>98%, 80%, and >98% respectively) (Supplement Table 1). But iodoacetamide was the only reagent that produced a CD4 derivative with gp120-binding affinity higher (6 folds) than the template D1D2F43C (reflected by lower IC₅₀) (FIG. 3 and Supplement Table 1).

Thus haloacetamide was picked as the primary starting module for construction of the thiol-reactive compound library. Some 5-nitro-2-pyridyldisulfides were also included. The initial library included 41 bromoacetamides (compounds 1-41) and 7 5-nitro-2-pyridyldisulfides (compounds 42-48), designed through a computer-assisted molecular complementarity search using GrowMol (Bohacek and McMartin 1994; Ripka, Satyshur et al. 2001). Based on the results from the first library, a second library of 32 bromoacetamides, 1 bromopropanone and 3 5-nitro-2-pyridyldisulfides (DN010-DN271) were further designed to complete the analysis of structure-activity relationships of cavity-filling probes.

### Chemistry

### D1D2F43C Derivatives

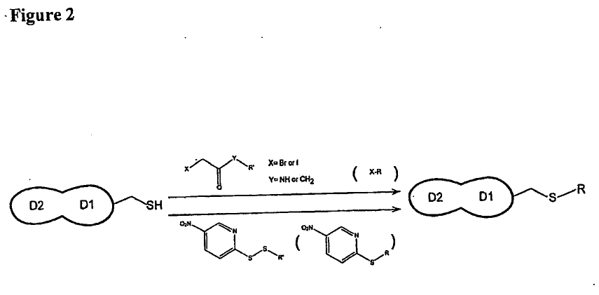

All D1D2F43C derivatives were generated through the nucleophilic reactions between thiolate anions of D1D2F43C and various electrophiles from the two compound libraries at pH 7-7.5 (FIG. 2). They were named D1D2F43C-“compound name” accordingly to the compounds used to derive them, e.g. D1D2F43C-1 was obtained by modifying D1D2F43C by with compound 1. Although 5-nitro-2-pyridyl disulfides have been shown to be able to generate mixed disulfides at acidic pH (Rabanal, DeGrado et al. 1996), we could only modify the free cysteine 43 of D1D2F43C using these disulfides at neutral pH (data not shown). 13 compounds out of altogether 94 compounds synthesized were not able to react well with D1D2F43C in our reaction condition (See Experimental Section) possibly due to their limited solubility in aqueous buffer. Rest (81) of the compounds all reacted efficiently with >80% completeness (Supplement Table 1).

### Overview of Binding Activities of CD4 Derivatives

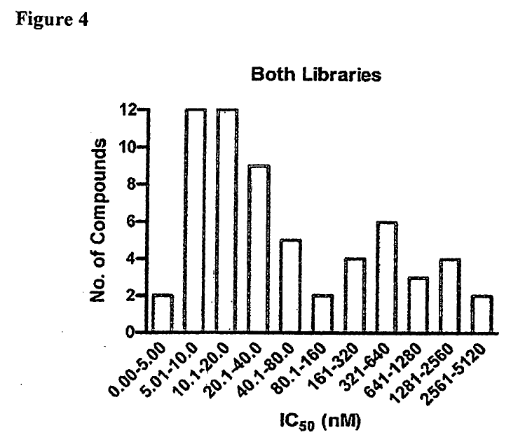

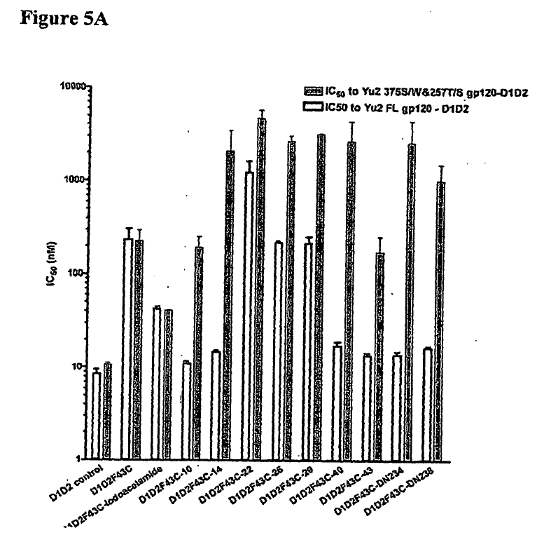

All 81 D1D2F43C derivatives were tested in competition ELISA for their abilities of inhibiting gp120-CD4 interaction. Although more than one third of the derivatives from initial library bound gp120 worse than the starting template D1D2F43C, all of the derivatives from the second library bound gp120 better than D1D2F43C (Table 1). Furthermore, majority of second-library (32 out of 36) derived protein had IC₅₀ lower than D1D2F43C-Iodoacetamide. The distribution of the IC₅₀ values of the D1D2F43C derivatives derived from both libraries was summarized in FIG. 4. There were large numbers of derivatives with IC₅₀ values lower than 20 nM. We were able to identify altogether 8 derivatives with IC₅₀ lower than that of D1D2 with best derivative (D1D2F43C-DN52) with IC₅₀ almost half of that of D1D2 (FIG. 3). The affinity of the derivatized D1D2F43C to gp120, however, reached a plateau with IC₅₀ around 4 nM even after extensive optimization.

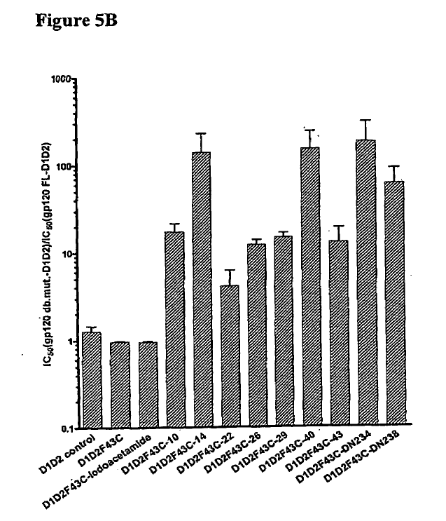

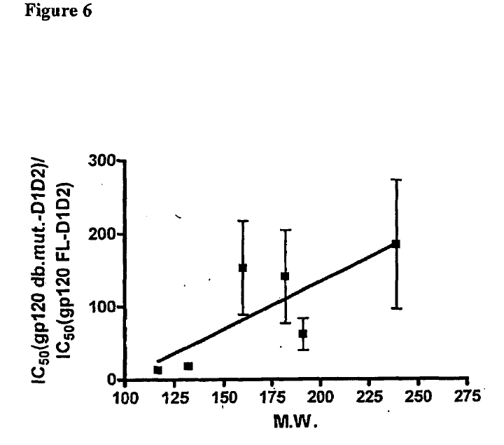

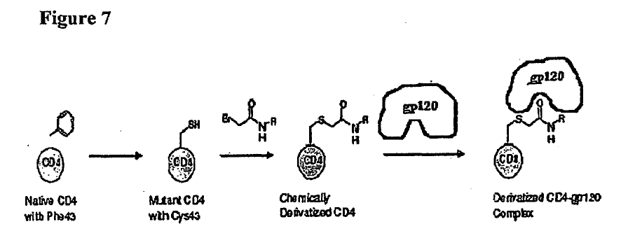

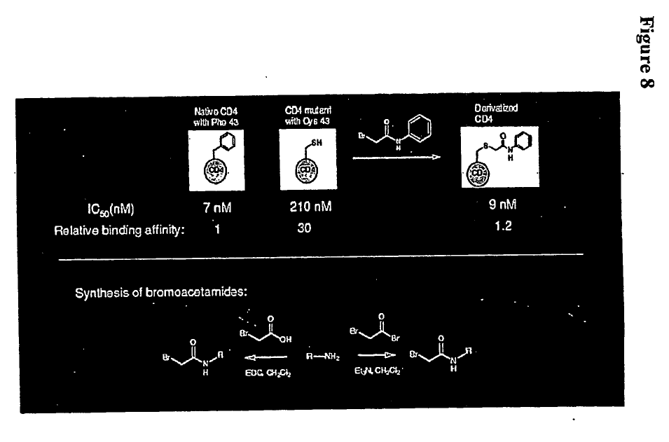

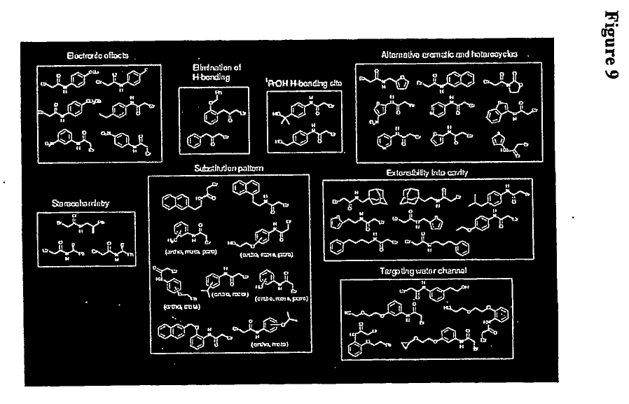

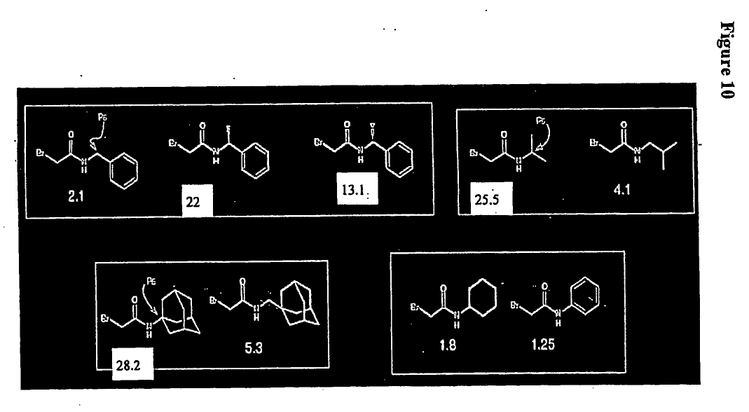

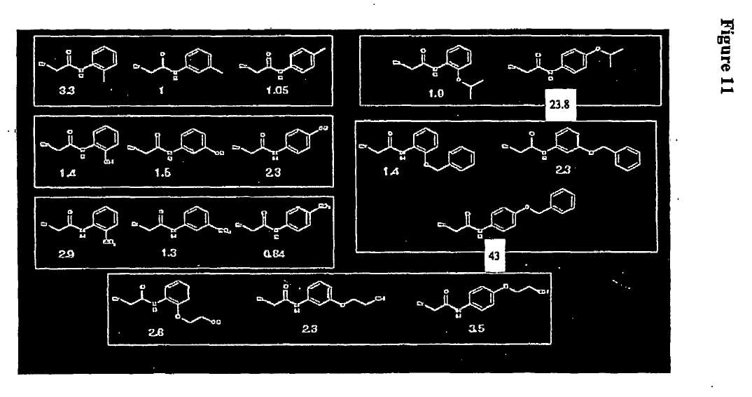

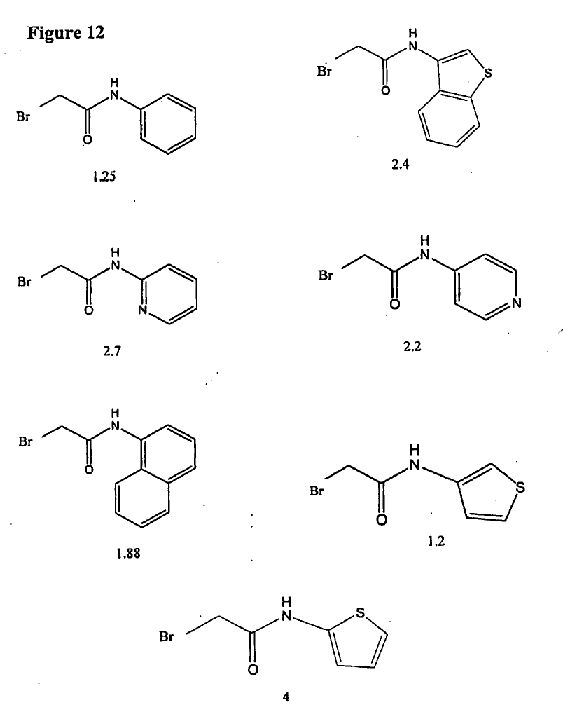

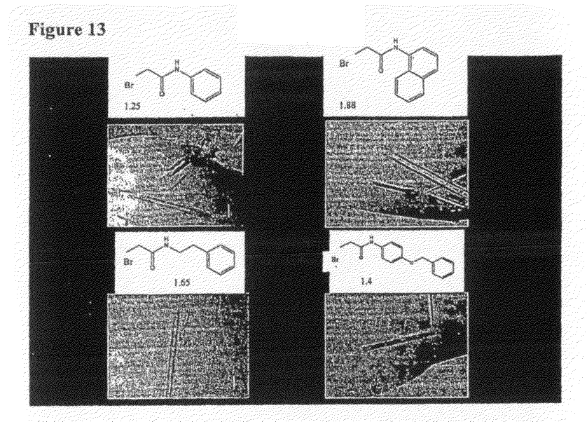

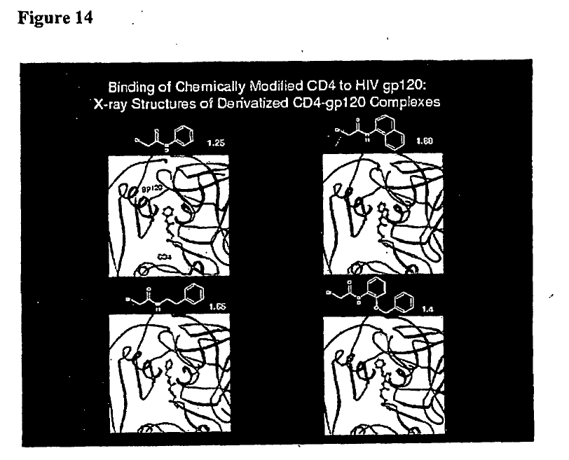

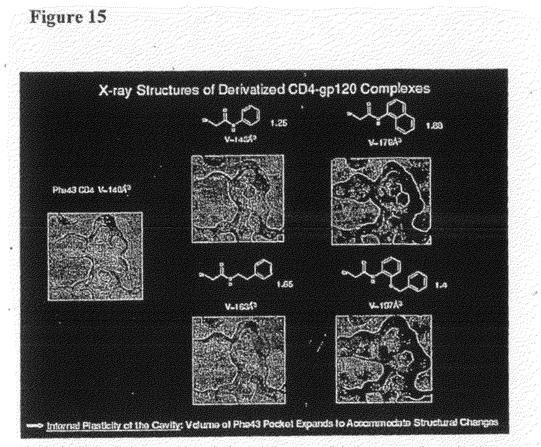

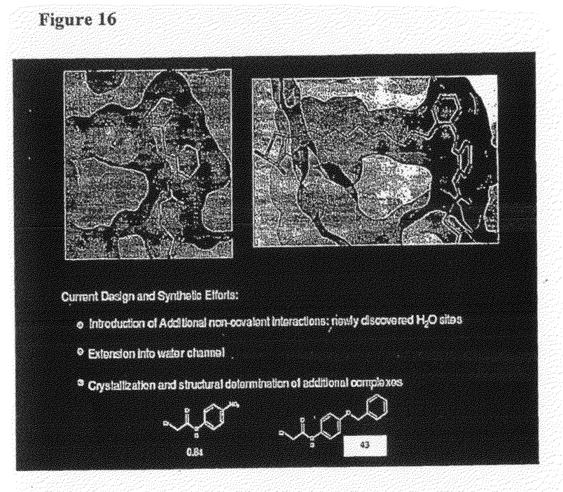

In order to carry out any valid SAR study on them, we first confirmed the presence of these D1D2F43C-attached compounds in the Phe43 cavity when they were bound by gp120 by measuring affinities of derivatives to a Phe43 cavity-filling mutant of gp120 in the competition ELISA. This gp120 mutant, namely gp120 S375W/T257S, has the cavity-lining residue serine 375 mutated to tryptophan, which fills the cavity (Xiang, Kwong et al. 2002). (The other mutation, T257S, is incorporated to stabilize gp120 S375W.) This cavity-filling mutant of gp120 bound D1D2 and D1D2F43C equally well as wild type gp120 did, as indicted by their identical IC₅₀ values in competition assay (FIG. 5). When a group of diverse D1D2F43C derivatives were tested, however, all except one derivatives had dramatically lower affinities (higher IC₅₀) to gp120 S375W/T257S than to wild type gp120 (FIG. 5). Lost of affinity to cavity-filled gp120 was not only true to the derivatives with nanomolar affinity such as D1D2F43C-10 but also true to the derivatives with micromolar affinity such as D1D2F43C-22. This proved that structurally diverse compounds were indeed specifically delivered into Phe43 cavity by being attached to D1D2F43C template. D1D2F43C-Iodoaetamide, the only derivative with equal affinity to gp120 S375W/T257S and wild type gp120, was not expected to reside in the cavity at all because of the small size of acetamide moiety. Among the derivatives with nanomolar affinities, a correlation can be found between the sizes of the compounds and the folds of IC₅₀ values of their derivatives increased from wild type gp120 to S375W/T257S gp120 (FIG. 6).

SAR of CD4-Attached Compounds on gp120

### N-alkyl-acetamide Derived D1D2F43C

The introduction of the small alkyl group, isopropyl (1), at the nitrogen of acetamide group, was not well tolerated by the cavity reflected by the increase of IC₅₀ by 5 folds from that of D1D2F43C-Iodoacetamide where only hydrogen was linked to acetamide nitrogen (Table 2.1). When bigger alkyl groups such as isobutyl (2) and 6-hydroxy-hexyl (9) were used, however, IC₅₀ values of the correspondingly modified D1D2F43C were back to the level comparable to, but not much better than that of D1D2F43C-Iodoacetamide. Even bulkier cyclic group, such as cyclohexyl group (3), increased the binding affinity more than 2 folds with an IC₅₀ of 13 nM, which was further reduced by half when an extra methylene linker was used between acetamide nitrogen and cyclohexyl group, i.e. cyclohexylmethyl (4). Presence of even bigger groups (6, DN-040 and 8) in cavity knocked down the binding affinity to micromolar level, suggesting the size limit of the cavity was probably reached.

### N-aryl-acetamide Derived D1D2F43C

Unlike alkyl groups, aryl groups were much more favored when attached to acetamide nitrogen. The highest binding affinity in this group (Table 2.2) was observed with D1D2F43C-10 (IC₅₀=7.76 nM) that has a phenyl group at acetamide nitrogen. Insertion of up to 3 methylene linkers between nitrogen and phenyl group (28, 40 and Dn-022) decreased binding slightly with ethyl being the best linker among the three. Interestingly, additional methylene branch to benzyl group at the first carbon atom linked to acetamide nitrogen resulted in a huge (5-10 folds) lost in affinity (29 and 30 compared to 28). Similarly to phenyl group, introduction of naphthalene group (14) at acetamide nitrogen was also favorable, but the additional linkers (32, Dn-149, 31, and Dn-152) between naphthalene and nitrogen were much less tolerated by the cavity. In light of the preference of phenyl and naphthyl like groups in the cavity, we also synthesized compounds containing 5-membered aromatic rings linked to acetamide. The results (Table 2.3) were very similar to the cases when phenyl was used except when acetamide nitrogen was linked to position 2 of thiophen (DN-155). Unlike D1D2F43C-DN-242, D1D2F43C-DN-155 had an IC₅₀ 3 times higher than that of its counterpart when phenyl was used (10). This was probably due to the different position of sulfur in thiophen, suggesting the unfavorable interaction between sulfur in DN-242 and the Phe43 cavity.

### Substituted N-phenyl-acetamide Derived D1D2F43C

Extensive substitutions on phenyl group of compound 10 were employed to screen for higher-affinity binders compared to parent compound 10 and to characterize the chemical preferences of Phe43 cavity.

Eighteen compounds were synthesized with various substitutions at para position (Table 2.4A). In general, small substitutions ranging from methyl (DN-183) to ethoxy (DN-185) groups had limited effect on the activities of the derivatized CD4, but larger group such as isopropoxy (22) and benzyloxy (26) groups reduced the affinities significantly. The highest binding affinities of this study were observed when compound 10 was substituted at para position by nitro (DN-052, 4.14 nM), isopropyl (12), or ethyl (DN-189) group. Interestingly, derivatives with hydroxy-containing substituents at para position always showed IC₅₀ values higher than that of similar derivatives with hydroxy group replaced by methyl group (DN-060 & DN-183, DN-199 & DN-189). This indicated that the electron-donating groups such as hydroxy are probably not favored at para position of the phenyl ring. An isopropanol was found in the Phe43 cavity in the refined 2.2 Å structure of HxBc2 gp120:D1D2:17B Fab (Kwong, Wyatt et al. 2000), suggesting a possible starting point for the design of cavity-binding compound. We tested this idea by adding an isopropanol group on the para position of phenyl group of compound 10. Surprisingly, the resulting compound DN-271 showed lower affinity to gp120 than the parent compound 10, indicating the disfavor of isopropanol group at this location of cavity.

A smaller collection of eight various groups were tested at meta position of phenyl ring of compound 10. A much higher tolerance for bulkier groups was noticed compared to the para position (Table 2.4B). Also small groups increased the affinity of the derivatives from the parent derivative (D1D2F43C-10) significantly less at meta position than at para position.

None of seven different substitutions at ortho position on phenyl group was able to enhance the affinity (Table 2.4C). Interestingly, the smallest group tested, methyl (DN-209), was the second to the least favored at this position with an IC₅₀ of 24.5 nM, yet a much bulkier group benzyloxy (DN-234) performed best at this position with an IC₅₀ of 8.87 nM, comparable to that of the parent compound.

### Role of Acetamide Moiety

D1D2F43C-Iodoacetamide, the simplest D1D2F43C derivatives containing only acetamide module, displayed much higher affinity to gp120 than D1D2F43C did (FIG. 3). We used compound 10 as a positive background to identify the gp120-binding elements in acetamide group. Either acetamide nitrogen or carbonyl oxygen of compound 10 was substituted to check their effect on the IC₅₀ of the correspondingly derived D1D2F43C. Two bromo-compounds were made, namely DN-171 (Supplement Table 1) and DN-180 (Table 2.5). DN-180, derived from compound 10 by mutating the acetamide nitrogen to the carbon, led to a CD4 derivative with gp120-binding affinity only 30% of that of D1D1F43C-10 (Table 2.5). Unfortunately, DN-171 failed to modify D1D2F43C possibly because that the removal of the carbonyl oxygen rendered the compound much less electrophilic for the reaction with cysteine to happen. The role of the carbonyl oxygen in gp120 binding, however, can still be indirectly examined from a disulfide derivative D1D2F43C-DN-146, which essentially resembled a carbonyl-oxygen deleted D1D2F43C-DN180 but had sulfur-carbon bond replaced by disulfide bond. The fact that these two derivatives had similar IC₅₀ in gp120-CD4 binding (Table 2.5) suggested that carbonyl oxygen is probably not essential for the interaction between acetamide moiety and gp120.

### Mixed-Disulfide Compounds Derivatized D1D2F43C

We were only able to study a very small selection of disulfide compounds due to their limited reactivity towards D1D2F43C. All of the derivatives contained aryl groups that were linked to cysteine 43 through disulfide bond (Table 2.6). The incorporation of most of these aryl groups significantly increased activities of derivatives in gp120 binding when compared with D1D2F43C. Similar to the SAR on the substituted N-phenyl-acetamide derived D1D2F43C, addition of isopropyl but not other bulkier group at para position of benzyl group in compound 42 slightly enhanced affinity (43).

### Discussion

The methods of identification of lead compounds or partial lead compounds (fragments) as antagonists of protein-protein interactions have expanded recently from the traditional high-throughput screening to more modern approaches such as NMR techniques, rational discovery and tethering (Gadek 2003). Among them, tethering (Erlanson, Braisted et al. 2000) also utilizes the chemical property of free thiol in cysteine, as we used in this study, to identify potential chemical fragments for specific binding pocket. In tethering, selected residues of the target protein around binding site are mutated to cysteines and used for screening a library of fragment molecules containing a disulfide moiety. The screening is done under partial reducing conditions so that only fragments really complimentary to the binding site can react with the free thiols. Compared to the method we used here, tethering can screen a large library of small fragment faster and have higher hit-finding rate by using more than one binding-site residues as tethering points. Tethering, however, is not practical in the case of identifying fragments targeting gp120 Phe43 cavity for two reasons. First, highly glycosylated gp120 does not permit the usage of mass spectrometry for identifying suitable fragments reacted with cysteines; second, weak-binding fragments may not be able to recognize and stabilize gp120 in the conformation that exhibit the phe43 cavity, resulting few or no hits. In this study, both difficulties were overcome by using D1D2F43C as tethering points for compounds. As the natural ligand for gp120, it stabilized Phe43 cavity in gp120; having no glycosylation sites, its derivatives were suitable for mass spectrometry study. In addition, usage of a gp120 ligand as the template in our method enables the quantitative study of structure-activity relations on the fragments, which would otherwise bind gp120 with weak affinity not suitable for accurate affinity determination using traditional binding assays.

When D1D2F43C template was used for tethering compounds, two potential binding sites on gp120 were available for small molecules. They were phe43 cavity and the binding site for the phenyl group of original phe43 residue. Chemical groups that separately targeted each site could work additively when combined properly. A good example comes from a recent study in rational design of CD4 mimetic CD4M33 (Martin, Stricher et al. 2003) where a biphenyl group instead of phenyl group at position corresponding to Phe43 of CD4 increases gp120-binding affinity of the mimetic by 6 folds. Crystal structure of core gp120 in complex with CD4M33 and 17b antibody shows that the addition phenyl binds inside the cavity where the bottom phenyl group superimposes well with the original phenyl group from phe43 residues (Huang, Stricher et al. 2005).

In this study, the usage of cysteine residue as the handle for chemoselective modification and screening of small molecules made it impossible to re-construct a phenyl group for occupying the original Phe43 binding position. Instead, the cysteine-reacting module (e.g. acetamide group for haloacetamides) of each type of cysteine modification reagents interacted with this site. Among all three kinds of electrophiles examined in this study (See Results), derivative of iodoacetamides had highest affinity to gp120 indicating the good complementarity of acetamide moiety at Phe43 binding site possible due to its relative small size as well as rigid and no-branching shape. Judging from its linear shape, it probably protruded out CDR2 loop of D1 domain and reached out to entrance of Phe43 cavity as much as the original phenyl group of original Phe43 residue. Its rather small “width” compared to phenyl ring, however, probably limited its capability of making extensive interactions with gp120 around cavity opening as seen with Phe43. This agreed with the observations that IC₅₀ of D1D2F43C-Iodoacetamide was 6 folds less than that of D1D2F43C but still 5 folds more than that of wild type D1D2 (FIG. 3 and Table 2.1). Among all atoms of acetamide group, nitrogen (NH) played a significant role (Table 2.5) in the interaction between acetamide moiety and gp120 probably thorough an interaction that CH2 is not capable of, such as hydrogen bonding. Addition of a cavity-targeting phenyl group onto acetamide moiety results in D1D2F43C-10 (Table 2.2) that bound gp120 5 times better than D1D2F43C-Iodoacetamide. The amount of affinity increased in filing the cavity by a phenyl group attached to acetamide moiety was comparable to that seen in CD4M33 (Martin, Stricher et al. 2003) indicating that the cavity-filling compounds could work in additively in gp120 binding with acetamide moiety by being directly connected to it.

On the other hand, modification by NEM decreased CD4's binding to gp120 significantly indicating that maleimide group was probably too bulky to be fitted at this site. In the case of disulfide compounds, i.e. 5-nitro-2-pyridyldisulfides, lack of proper control compound could not give a direct answer as to how well the phenyl site was occupied but the flexible nature of disulfide bond and the sulfur-carbon bond in disulfanly-benzyl moiety probably rendered it less favored by Phe43 binding site than acetamide moiety. Another disulfides-derived CD4, D1D2F43C-DTNB did not bind gp120 well not because of the disulfide linkage but probably because of the large size and extremely hydrophilic nature of 3-Carboxy-4-nitrophenyl group. This was supported by the high affinities of 5-nitro-2-pyridyldisulfides derived D1D2F43C (Table 2.6).

Most efforts in this study had been taken to characterize the chemical preference of the compounds in binding the Phe43 cavity by studying the structure-activity relationships of altogether 81 D1D2-compound hybrids. Despite of dramatic differences in their sizes, shapes, chemical properties, and affinities to gp120, all compounds presented by D1D2F43C template resided Phe43 cavity (FIG. 5). Although it is not clear whether the whole compounds could all fit in the cavity, especially the ones that were much bigger than the size of the cavity derived from known structures of core gp120 (Kwong, Wyatt et al. 1998; Kwong, Wyatt et al. 2000) and had IC₅₀ values of the corresponding derivatives much less than D1D2F43C-Iodoacetamine, gp120 must had certain degree of plasticity and underwent necessary structure rearrangement to make Phe43 cavity big enough to accommodate bulky compounds such as DN-234 (Table 2.4C) whose derivatives had high affinities to gp120.

In the SAR study of N-alkyl-acetamide derived D1D2F43C (Table 2.1), isobutyl group was found to be much more favored than a smaller isopropyl group, which branched at the first carbon linked to acetamide nitrogen. This suggested that Phe43 cavity may had a relatively narrow opening near the binding site of acetamide nitrogen. The side groups that branched out from acetamide nitrogen may have repulsive steric interaction with the residues around the cavity neck region while non-branching groups may be accommodated well. This also agreed with the observation that insertion of a methylene linker between nitrogen and a bulky group just as cyclohexyl or adamantan group enhanced the affinity.

Crystal structures of CD4-bound core gp120 also show the existence of a cavity entrance narrower than the body of cavity (Kwong, Wyatt et al. 1998; Kwong, Wyatt et al. 2000), suggesting this narrow entrance of the cavity is preserved from full length gp120 to core gp120. Modeling of cysteine-acetamide on the D1D2 scaffold positioned acetamide nitrogen close to the cavity neck (data not shown), consistent with the finding from the SAR data.

The introduction of isobutyl group at acetamide nitrogen, however, only had marginally positive effect on affinity probably due to limited van der Waals interactions. On the contrary, cyclohexyl group, although had bulky branches, affected affinity positively possibly by providing more van der Waals contacts.

Aromatic groups such as phenyl and thiophen (Tables 2.2 and 2.3) were shown to be the most favorable groups when linked to acetamide nitrogen at the cavity entrance probably because they not only had capability of engaging a lot of van der Waals interactions like cyclohexyl, but also had rigid and planar shapes that could be accommodated much better at the cavity neck than cyclohexyl group. Additional methylene linkers between nitrogen and phenyl or thiophen group, unlike in the case of alkyl group, affected binding adversely. One plausible explanation was that phenyl group fitted the narrow cavity entrance well but could no longer maintain all the favorable van der Waals contacts once in the broader space inside the cavity due to the insertion of methylene linkers. Again, in agreement with the presence of a narrow cavity neck, additional methyl branch of benzyl group at the first carbon atom linked to acetamide nitrogen resulted in a dramatic affinity lost (Compound 29 and 30, Table 2.2).

Substitutions on the para position of phenyl group of compound 10 (Table 2.4) were found to be most effective in affinity enhancement. Best substituents were identified as nitro (DN-052) and isopropyl (12) groups with IC₅₀ of 4.14 and 4.84 nM respectively. These two groups' different-electronic properties but similar molecular shape and similar effect in binding suggested that they may enhance binding solely through hydrophobic interaction with cavity residues. Similarly to phenyl group, insertion of methylene bridges for these substituted phenyl groups weakened binding (Supplemental Table 1: 12 & 25; DN-052 & 36) possibly by breaking the perfect shape complementation of these substituted phenyl groups to Phe43 cavity. These evidences, together with the observation on the disfavor of hydroxy group on the phenyl ring and sulfur on thiophen group (Table 2.3 and 2.4), indicated that van der Waals interactions may dominate the recognition of the compounds within cavity. This is consistent with the crystal structure of core gp120 in which the Phe cavity is primarily lined by hydrophobic residues (Kwong, Wyatt et al. 1998; Kwong, Wyatt et al. 2000). Although main chain atoms of the cavity-enclosing residues of gp120, such amide nitrogens in residue 376 and 377 or carbonyl oxygen of residue 375, could in principle participate in hydrophilic interactions, based on this study this appeared not to be the case. The finding of an isopropanol occupying the cavity in the crystal structure of core HxBc2 gp120 bound with D1D2 (Kwong, Wyatt et al. 2000) was also not in contradictory to the observed preference of hydrophobic compounds in the cavity since the hydroxy group of isopropanol is hydrogen-bonded to two water molecules but not any residue from the gp120 in the crystal structure.

Substitutions on meta and ortho positions on compound 10's phenyl ring did not work as well as that on para position. But much larger groups (Table 2.4) such as groups seen in compound DN-234 were able to fit in the cavity without affecting binding affinity at all, suggesting they may bind in the cavity in different modes from the para-substituted phenyl groups and their binding in cavity may involve inducing gp120's structural rearrangement and expansion of cavity region.

In summary, the structure-activity relationship (SAR) study of derivatized D1D2F43C binding to gp120 revealed that the narrow entrance of cavity seen in the structure of core gp120 bound by CD4 was also preserved in full length gp120 bound by derivatized CD4 but the cavity itself displayed significant plasticity in binding different compounds. Van der Waals interactions were found to be primarily responsible for the recognition of compounds by cavity while hydrophilic interactions may play a role around cavity entrance as seen with nitrogen of acetamide group.

Plasticity at protein-protein interface has been observed and studied (DeLano, Ultsch et al. 2000; Ma, Shatsky et al. 2002). Significant adaptability of Phe43 cavity, however, was not expected because of the presence of D1D2 scaffold, which pre-fixed gp120 at a CD4-bound substrate. This may explain the relative small gain in affinity by addition of isopropyl/nitro groups at the para position of phenyl group of compound 10 (Table 2.4) as well as the IC₅₀ plateau of 4 nM reached after optimization: addition of favorable compounds in cavity may still result in the redistribution of gp120 population to a substate that is not permitted or favored by D102F43C template. This penalty from occupying the cavity that is intrinsic flexible but stabilized by D1D2 scaffold may prevent the further increase in affinity of derivatized D1D2F43C. A smaller CD4 scaffold such as mimetic may be a reasonable template for further optimization of cavity-binding fragment because it may structure gp120 less. D1D2 template, however, still has its irreplaceable values in that derivatized D1D2F43C can be readily used for structural study while most of CD4 mimetic can not except for CD4M33, of which the structure was recently solved in complex with gp120 (Huang, Stricher et al. 2005).

From the therapeutic aspect, it is interesting to know what kind of conformation gp120 adopts when Phe43 cavity is occupied by derivatized D1D2F43C. Are derivatized D1D2F43C CD4 agonists or CD4 antagonists? It is obvious that CD4 antagonists can be very useful as entry inhibitor by preventing the virus attachment, CD4 agonists have also been shown to be antiretroviral in vivo (Vermeire and Schols 2005), possibly by either blocking coreceptor binding sterically or by fixing gp120 in the CD4-bound conformation recognizable by immune system. They also can be particularly useful tools in the development of antibodies and vaccines against gp120 (Kang, Hariharan et al. 1994).

CD4 mimics, such as CD4-IgG, are CD4 agonists that stabilize gp120 in CD4-bound co-receptor-ready state. Derivatized D1D2F43C only differed from the CD4 mimics by occupying the Phe43 cavity, which probably only exits in gp120 upon CD4 binding. Does that necessarily determine gp120 in a CD4-bound state? Although biochemical data from a cavity-filling mutant of gp120 (S375W) suggested that gp120 adopts a conformation more resembling the co-receptor ready state (Xiang, Kwong et al. 2002), situation could be different when the unit that plugs in the cavity does not come from gp120 itself because it may inhibit the transduction of potential conformational change by locking gp120 in a particular conformation. Indirect evidences can be found from studies on CD4M33—a CD4 mimetic with an additional phenyl group attached to Phe33 position (equivalent to Phe43 of CD4) in CD4F23 mimetic. It has been shown by thermodynamic study (Huang, Stricher et al. 2005) that CD4M33 introduces much less structural rearrangement in gp120 reflected by entropic changes than CD4F23 and D1D2. Ability of promoting the binding of gp120 to CCR5 by CD4M33 is also 6 folds less than that of CD4 (Martin, Stricher et al. 2003). Both evidences strongly support the notion that gp120 with occupied Phe43 pocket may adopt a conformation different from either free or CD4-bound state.

In conclusion, we had constructed a library of D1D2-compound hybrids and presented here the structure-activity relationships of the integrated compound binding to the Phe43 cavity in gp120. The collection of structure-activity relationships of these cavity probes provided us insight on the detailed shape, conformational adaptability and chemical preference of Phe43 pocket, which could benefit the further transformation of the identified compounds into efficient gp120-CD4 inhibitors by incorporating them onto either smaller scaffold, such as miniproteins, or small molecules by fragment assembly. The unique utilization of a reactive ligand template (CD4) in screening compounds for targeting a receptor (gp120) site at the ligand-receptor interface may be applied to other system when the stabilization of targeting site on the receptor requires the presence of the ligand.

### REFERENCES

Allan, J. S., J. E. Coligan, et al. (1985). “Major glycoprotein antigens that induce antibodies in AIDS patients are encoded by HTLV-III.” Science 228(4703): 1091-4.
Allaway, G. P., K. L. Davis-Bruno, et al. (1995). “Expression and characterization of CD4-IgG2, a novel heterotetramer that neutralizes primary HIV type 1 isolates.” AIDS Res Hum Retroviruses 11(5): 533-9.
Arthos, J., K. C. Deen, et al. (1989). “Identification of the residues in human CD4 critical for the binding of HIV.” Cell 57(3): 469-81.
Barre-Sinoussi, F., J. C. Chermann, et al. (1983). “Isolation of a T-lymphotropic retrovirus from a patient at risk for acquired immune deficiency syndrome (AIDS).” Science 220(4599): 868-71.
Bohacek, R. S. and C. McMartin (1994). “Multiple Highly Diverse Structures Complementary to Enzyme Binding Sites: Results of Extensive Application of a de Novo Design Method Incorporating Combinatorial Growth.” Journal of the American Chemical Society 116(13): 5560-71.
Boyd, M. R., K. R. Gustafson, et al. (1997). “Discovery of cyanovirin-N, a novel human immunodeficiency virus-inactivating protein that binds viral surface envelope glycoprotein gp120: potential applications to microbicide development.” Antimicrob Agents Chemother 41(7): 1521-30.
Burton, D. R., J. Pyati, et al. (1994). “Efficient neutralization of primary isolates of HIV-1 by a recombinant human monoclonal antibody.” Science 266(5187): 1024-7.
Chan, D. C., D. Fass, et al. (1997). “Core structure of gp41 from the HIV envelope glycoprotein.” Cell 89(2): 263-73.
Chapman, M. S., I. Minor, et al. (1991). “Human rhinovirus 14 complexed with antiviral compound R 61837.” J Mol Biol 217(3): 455-63.
Chen, B., E. M. Vogan, et al. (2005). “Determining the structure of an unliganded and fully glycosylated SIV gp120 envelope glycoprotein.” Structure (Camb) 13(2): 197-211.
Chen, B., E. M. Vogan, et al. (2005). “Structure of an unliganded simian immunodeficiency virus gp120 core.” Nature 433(7028): 834-41.
Cochran, A. G. (2000). “Antagonists of protein-protein interactions.” Chem Biol 7(4): R85-94.
Daigleish, A. G., P. C. Beverley, et al. (1984). “The CD4 (T4) antigen is an essential component of the receptor for the AIDS retrovirus.” Nature 312(5996): 763-7.
De Clercq, E. (2005). “Emerging anti-HIV drugs.” Expert Opin Emerg Drugs 10(2): 241-73.
DeLano, W. L., M. H. Ultsch, et al. (2000). “Convergent solutions to binding at a protein-protein interface.” Science 287(5456): 1279-83.
Dey, B., C. S. Del Castillo, et al. (2003). “Neutralization of human immunodeficiency virus type 1 by sCD4-17b, a single-chain chimeric protein, based on sequential interaction of gp120 with CD4 and coreceptor.” J Virol 77(5): 2859-65.
Erlanson, D. A., A. C. Braisted, et al. (2000). “Site-directed ligand discovery.” Proc Natl Acad Sci USA 97(17): 9367-72.
Ferrer, M. and S. C. Harrison (1999). “Peptide ligands to human immunodeficiency virus type 1 gp120 identified from phage display libraries.” J Virol 73(7): 5795-802.
Furuta, R. A., C. T. Wild, et al. (1998): “Capture of an early fusion-active conformation of HIV-1 gp41.” Nat Struct Biol 5(4): 27 6-9.
Gadek, T. R. (2003). “Strategies and methods in the identification of antagonists of protein-protein interactions.” Biotechniques Suppl: 21-4.
Gallo, R. C., S. Z. Salahuddin, et al. (1984). “Frequent detection and isolation of cytopathic retroviruses (HTLV-III) from patients with AIDS and at risk for AIDS.” Science 224(4648): 500-3.
Guo, Q., H. T. Ho, et al. (2003). “Biochemical and genetic characterizations of a novel human immunodeficiency virus type 1 inhibitor that blocks gp120-CD4 interactions.” J Virol 77(19): 10528-36.
Helseth, E., U. Olshevsky, et al. (1991). “Human immunodeficiency virus type 1 gp120 envelope glycoprotein regions important for association with the gp41 transmembrane glycoprotein.” J Virol 65(4): 2119-23.
Huang, C. C., F. Stricher, et al. (2005). “Scorpion-toxin mimics of CD4 in complex with human immunodeficiency virus gp120 crystal structures, molecular mimicry, and neutralization breadth.” Structure (Camb) 13(5): 755-68.
Huang, C. C., M. Tang, et al. (2005). “Structure of a V3-containing HIV-1 gp120 core.” Science 310(5750): 1025-8.
Kang, C. Y., K. Hariharan, et al. (1994). “Immunization with a soluble CD4-gp120 complex preferentially induces neutralizing anti-human immunodeficiency virus type 1 antibodies directed to conformation-dependent epitopes of gp120.” J Virol 68(9): 5854-62.
Kilby, J. M. and J. J. Eron (2003). “Novel therapies based on mechanisms of HIV-1 cell entry.” N Engl J Med 348(22): 2228-38.
Klatzmann, D., E. Champagne, et al. (1984). “T-lymphocyte T4 molecule behaves as the receptor for human retrovirus LAV.” Nature 312(5996): 767-8.
Kowalski, M., J. Potz, et al. (1987). “Functional regions of the envelope glycoprotein of human immunodeficiency virus type 1.” Science 237(4820): 1351-5.
Kwong, P. D., R. Wyatt, et al. (2000). “Structures of HIV-1 gp120 envelope glycoproteins from laboratory-adapted and primary isolates.” Structure Fold Des 8(12): 1329-39.
Kwong, P. D., R. Wyatt, et al. (1998). “Structure of an HIV gp120 envelope glycoprotein in complex with the CD4 receptor and a neutralizing human antibody.” Nature 393(6686): 648-59.
Lin, P. F., W. Blair, et al. (2003). “A small molecule HIV-1 inhibitor that targets the HIV-1 envelope and inhibits CD4 receptor binding.” Proc Natl Acad Sci USA 100(19): 11013-8.
Lu, M., S. C. Blacklow, et al. (1995). “A trimeric structural domain of the HIV-1 transmembrane glycoprotein.” Nat Struct Biol 2(12): 1075-82.
Ma, B., M. Shatsky, et al. (2002). “Multiple diverse ligands binding at a single protein site: a matter of pre-existing populations.” Protein Sci 11(2): 184-97.
Madani, N., A. L. Perdigoto, et al. (2004). “Localized changes in the gp120 envelope glycoprotein confer resistance to human immunodeficiency virus entry inhibitors BMS-806 and #155.” J Virol 78(7): 3742-52.
Martin, L., F. Stricher, et al. (2003). “Rational design of a CD4 mimic that inhibits HIV-1 entry and exposes cryptic neutralization epitopes.” Nat Biotechnol 21(1): 71-6.
Milligan, G. N., C. F. Chu, et al. (2004). “Effect of candidate vaginally-applied microbicide compounds on recognition of antigen by CD4+ and CD8+ T lymphocytes.” Biol Reprod 71(5): 1638-45.
Modrow, S., B. H. Hahn, et al. (1987). “Computer-assisted analysis of envelope protein sequences of seven human immunodeficiency virus isolates: prediction of antigenic epitopes in conserved and variable regions.” J Virol 61(2): 570-8.
Myszka, D. G., R. W. Sweet, et al. (2000). “Energetics of the HIV gp120-CD4 binding reaction.” Proc Natl Acad Sci USA 97(16): 9026-31.
Rabanal, F., W. F. DeGrado, et al. (1996). “Use of 2,2′-dithiobis(5-nitropyridine) for the heterodimerization of cysteine containing peptides. Introduction of the 5-nitro-2-pyridinesulfenyl group.” Tetrahedron Letters 37(9): 1347-50.
Rees, D. C., M. Congreve, et al. (2004). “Fragment-based lead discovery.” Nat Rev Drug Discov 3(8): 660-72.
Richman, D. D. (2001). “HIV chemotherapy.” Nature 410(6831): 995-1001.
Ripka, A. S., K. A. Satyshur, et al. (2001). “Aspartic protease inhibitors designed from computer-generated templates bind as predicted.” Organic letters 3(15): 2309-12.
Robey, W. G., B. Safai, et al. (1985). “Characterization of envelope and core structural gene products of HTLV-III with sera from AIDS patients.” Science 228(4699): 593-5.
Rusconi, S., M. Moonis, et al. (1996). “Naphthalene sulfonate polymers with CD4-blocking and anti-human immunodeficiency virus type 1 activities.” Antimicrob Agents Chemother 40(1): 234-6.
Ryu, S. E., P. D. Kwong, et al. (1990). “Crystal structure of an HIV-binding recombinant fragment of human CD4.” Nature 348(6300): 419-26.
Ryu, S. E., A. Truneh, et al. (1994). “Structures of an HIV and MHC binding fragment from human CD4 as refined in two crystal lattices.” Structure 2(1): 59-74.
Si, Z., N. Madani, et al. (2004). “Small-molecule inhibitors of HIV-1 entry block receptor-induced conformational changes in the viral envelope glycoproteins.” Proc Natl Acad Sci USA 101(14): 5036-41.
Smith, A. B., 3rd, S. N. Savinov, et al. (2002). “Peptide-small molecule hybrids via orthogonal deprotection-chemoselective conjugation to cysteine-anchored scaffolds. A model study.” Org Lett 4(23): 4041-4.
Smith, D. H., R. A. Byrn, et al. (1987). “Blocking of HIV-1 infectivity by a soluble, secreted form of the CD4 antigen.” Science 238(4834): 1704-7.
Starcich, B. R., B. H. Hahn, et al. (1986). “Identification and characterization of conserved and variable regions in the envelope gene of HTLV-III/LAV, the retrovirus of AIDS.” Cell 45(5): 637-48.
Tan, K., J. Liu, et al. (1997). “Atomic structure of a thermostable subdomain of HIV-1 gp41.” Proc Natl Acad Sci USA 94(23): 12303-8.
Trkola, A., T. Dragic, et al. (1996). “CD4-dependent, antibody-sensitive interactions between HIV-1 and its co-receptor CCR-5.” Nature 384(6605): 184-7.
Vermeire, K. and D. Schols (2005). “Anti-HIV agents targeting the interaction of gp120 with the cellular CD4 receptor.” Expert Opin Investig Drugs 14(10): 1199-212.
Wang, J. H., Y. W. Yan, et al. (1990). “Atomic structure of a fragment of human CD4 containing two immunoglobulin-like domains.” Nature 348(6300): 411-8.
Wang, T., Z. Zhang, et al. (2003). “Discovery of 4-benzoyl-1-[(4-methoxy-1H-pyrrolo[2,3-b]pyridin-3-yl)oxoacetyl]-2-(R)-methylpiperazine (BMS-378806): a novel HIV-1 attachment inhibitor that interferes with CD4-gp120 interactions.” J Med Chem 46(20): 4236-9.
Weissenhorn, W., A. Dessen, et al. (1997). “Atomic structure of the ectodomain from HIV-1 gp41.” Nature 387(6631): 426-30.
Wu, H., P. D. Kwong, et al. (1997). “Dimeric association and segmental variability in the structure of human CD4.” Nature 387(6632): 527-30.
Wu, L., N. P. Gerard, et al. (1996). “CD4-induced interaction of primary HIV-1 gp120 glycoproteins with the chemokine receptor CCR-5.” Nature 384(6605): 179-83.
Wyatt, R., P. D. Kwong, et al. (1998). “The antigenic structure of the HIV gp120 envelope glycoprotein.” Nature 393(6686): 705-11.
Xiang, S. H., P. D. Kwong, et al. (2002). “Mutagenic stabilization and/or disruption of a CD4-bound state reveals distinct conformations of the human immunodeficiency virus type 1 gp120 envelope glycoprotein.” J Virol 76(19): 9888-99.
Zhang, A., R. G. Nanni, et al. (1993). “Structure determination of antiviral compound SCH 38057 complexed with human rhinovirus 14.” J Mol Biol 230(3): 857-67.

TABLE 1
Summary of the IC₅₀ values of the D1D2F43C derivatives
from both compound libraries.

| | | No. Of the Compounds | | |
|---|---|---|---|---|
| | | Initial | | |
| | | Library | New Library | Both Libraries |
| Reacted | | 45 | 36 | 81 |
| IC₅₀ values of | 4.14-30.0 | 21 | 32 | 53 |
| derivatized | 30.1-200 | 7 | 4 | 11 |
| D1D2F43C (nM) | >200 | 17 | 0 | 17 |

| | | | |
|---|---|---|---|
| Unreacted | 3 | 10 | 13 |
| Total | 48 | 46 | 94 |

SUPPLEMENT TABLE 1
Chemical structures of the cysteine modifying
compounds, completeness of the corresponding modification and
corrected IC₅₀ (except disulfide compounds indicated in bold)
values of the correspondingly derivatized D1D2 to the binding of
D1D2 and YU2 gp120. The correction factors for converting
measured IC₅₀ to corrected IC₅₀ are also listed. The IC50 values
are presented as the mean ± SD values from two to three
independent experiments.

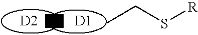

| | | | | IC50 ± SD of |
|---|---|---|---|---|
| | | Complete- | Correction | Derivatized D1D2 |
| Compound | R | ness (%) | Factor | (nM) |
| Iodo- acetamide | 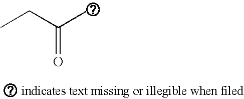 | >98 | 0.98 | 33.3 ± 5.5 |
| NEM | 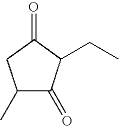 | >98 | N/A | 3.22 × 10³ ± 2.33 × 10³ |
| DTNB | 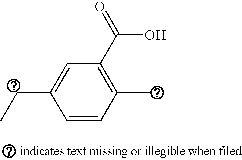 | 80 | N/A | 3.58 × 10³ ± 1.64 × 10³ |
| 1 | 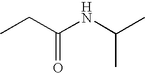 | >98 | 1.00 | 184 ± 39 |
| 2 | 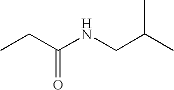 | >98 | 1.00 | 29.4 ± 2.2 |
| 3 | 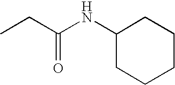 | >98 | 1.00 | 13.0 ± 2.1 |
| 4 | 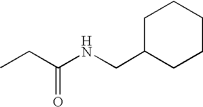 | >98 | 1.00 | 7.79 ± 2.08 |
| 5 | 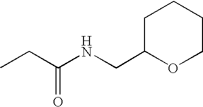 | >98 | 1.00 | 17.0 ± 2.5 |
| 6 | 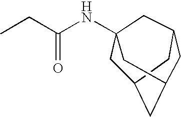 | >98 | 1.00 | 223 ± 49 |
| 7 | 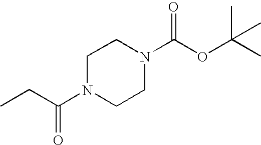 | >98 | 1.00 | 1.86 × 10³ ± 0.32 × 10³ |
| 8 | 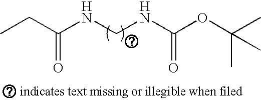 | >98 | 1.00 | 1.68 × 10⁺± 0.50 × 10³ |
| 9 | 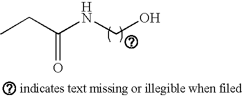 | >98 | 1.00 | 33.0 ± 9.7 |
| 10 | 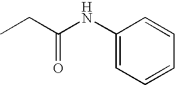 | >98 | 0.88 | 7.76 ± 0.83 |
| 11 | 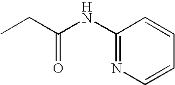 | >99 | 0.69 | 12.3 ± 0.77 |
| 12 | 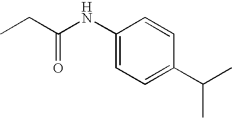 | >98 | 0.85 | 4.84 ± 0.83 |
| 13 | 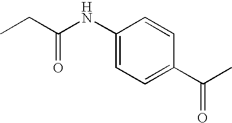 | >98 | 0.51 | 8.14 ± 0.4 |
| 14 | 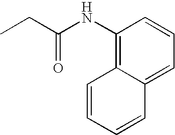 | >98 | 0.75 | 10.4 ± 0.8 |
| 15 | 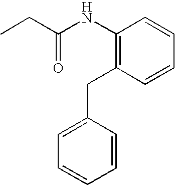 | >98 | 1.00 | 51.9 ± 6.3 |
| 16 | 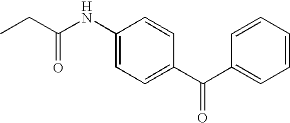 | >98 | 0.52 | 46.2 ± 1.3 |
| 17 | 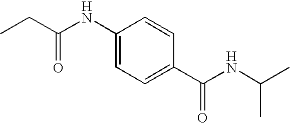 | >98 | 0.50 | 402 ± 68 |
| 18 | 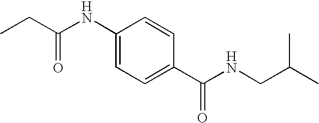 | >98 | 0.59 | 4.18 × 10³ ± 2.63 × 10³ |
| 19 | 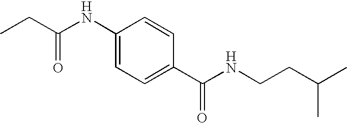 | >98 | 0.56 | 3.34 × 10³ ± 0.51 × 10³ |
| 20 | 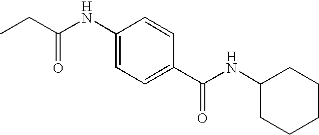 | >98 | 0.90 | 921 ± 206 |
| 21 | 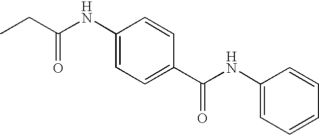 | >98 | 0.69 | 456 ± 89 |
| 22 | 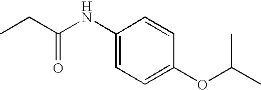 | >98 | 0.92 | 1.50 × 10³ ± 0.33 × 10³ |
| 23 | 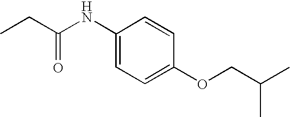 | >98 | 0.91 | 637 ± 202 |
| 24 | 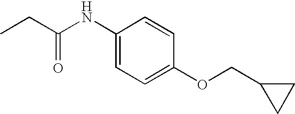 | >98 | 0.93 | 764 ± 207 |
| 25 | 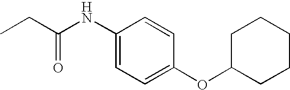 | >98 | 0.95 | 470 ± 205 |
| 26 | 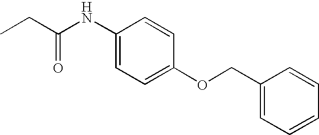 | >98 | 0.93 | 290 ± 84 |
| 27 | 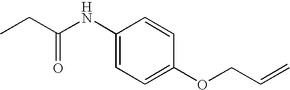 | >98 | 0.92 | 1.21 × 10³ ± 0.34 × 10³ |
| 28 | 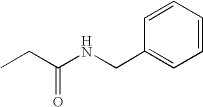 | >98 | 1.00 | 15.2 ± 0.7 |
| 29 | 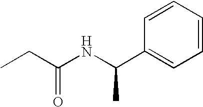 | >98 | 1.00 | 88.7 ± 1.5 |
| 30 | 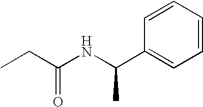 | >98 | 1.00 | 166 ± 22 |
| 31 | 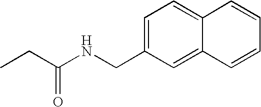 | >98 | 0.87 | 12.2 ± 0.8 |
| 32 | 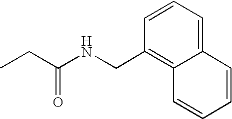 | >98 | 0.72 | 75.7 ± 11.2 |
| 33 | 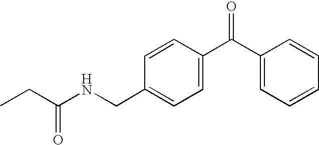 | >98 | 0.56 | 421 ± 9 |
| 34 | 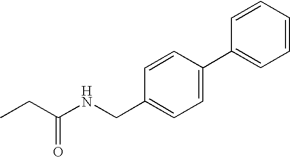 | 25 | N/A | N/A |
| 35 | 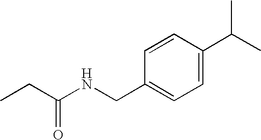 | >98 | 1.00 | 7.95 ± 0.09 |
| 36 | 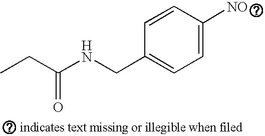 | >98 | 0.67 | 14.9 ± 0.0 |
| 37 | 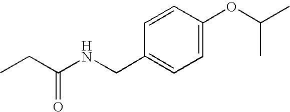 | >98 | 0.94 | 25.7 ± 6.4 |
| 38 | 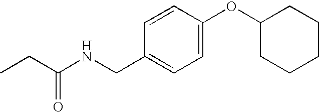 | >98 | 0.95 | 350 ± 41 |
| 39 | 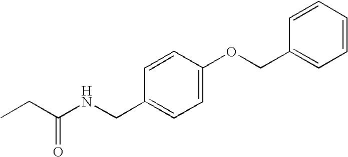 | 10 | N/A | N/A |
| 40 | 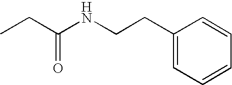 | >98 | 1.00 | 10.6 ± 2.9 |
| 41 | 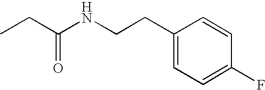 | >98 | 1.00 | 10.6 ± 0.7 |
| 42 | 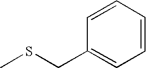 | >98 | N/A | 11.5 ± 1.8 |
| 43 | 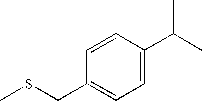 | >98 | N/A | 9.14 ± 1.99 |
| 44 | 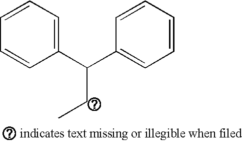 | >98 | N/A | 1.38 × 10³ ± 0.10 ± 10³ |
| 45 | 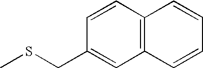 | >98 | N/A | 14.3 ± 0.6 |
| 46 | 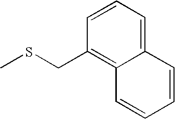 | 20 | N/A | N/A |
| 47 | 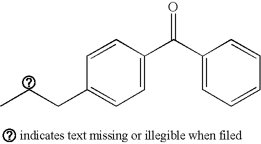 | 90 | N/A | 27.0 ± 1.6 |
| 48 | 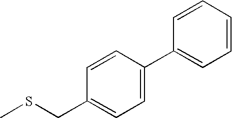 | >96 | N/A | 14.9 ± 0.0 |
| DN-010 | 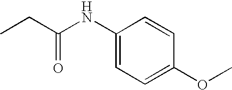 | <30 | 0.75 | N/A |
| DN-012 | 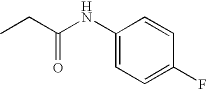 | >98 | 0.89 | 7.08 ± 1.24 |
| DN-022 | 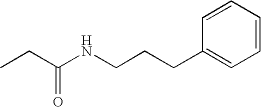 | >98 | 1.00 | 16.4 ± 2.6 |
| DN-026 | 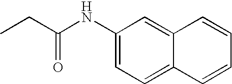 | <30 | 0.78 | N/A |
| DN-028 | 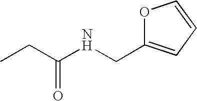 | >98 | 1.00 | 18.3 ± 2.1 |
| DN-030 | 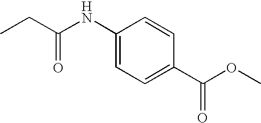 | >98 | 0.52 | 9.87 ± 0.67 |
| DN-034 | 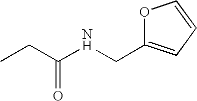 | >98 | 1.00 | 18.8 ± 0.5 |
| DN-040 | 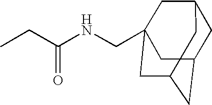 | >98 | 1.00 | 42.3 ± 5.9 |
| DN-052 | 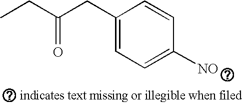 | >98 | 0.72 | 4.14 ± 0.03 |
| DN-054 | 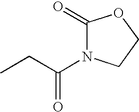 | <30 | N/A | N/A |
| DN-060 | 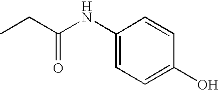 | >98 | 0.75 | 11.4 ± 0.2 |
| DN-064 | 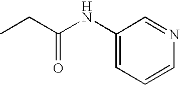 | 0 | N/A | N/A |
| DN-143 | 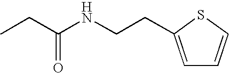 | <30 | N/A | N/A |
| DN144 | 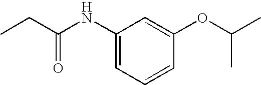 | <30 | N/A | N/A |
| DN-146 | 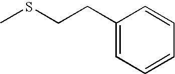 | >98 | N/A | 24.7 ± 8.6 |
| DN-149 | 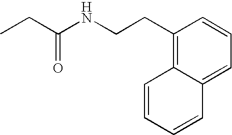 | >98 | 0.83 | 69.3 ± 1.8 |
| DN150 | 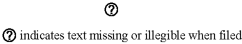 | <30 | N/A | N/A |
| DN-152 | 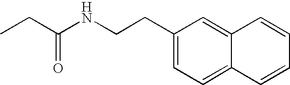 | 80 | 0.85 | 146 ± 4 |
| DN-155 | 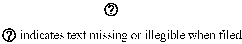 | >98 | 0.70 | 18.2 ± 1.8 |
| DN-170 | 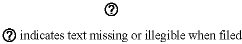 | >98 | 0.81 | 13.1 ± 0.5 |
| DN-171 | 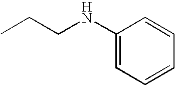 | 0 | N/A | N/A |
| DN-179 | 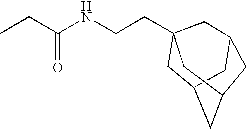 | <30 | N/A | N/A |
| DN-180 | 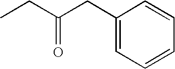 | >98 | 1.00 | 26.4 ± 3.9 |
| DN-182 | 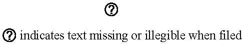 | <30 | N/A | N/A |
| DN-183 | 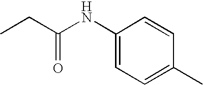 | >98 | 0.79 | 7.07 ± 1.51 |
| DN-185 | 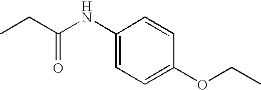 | >98 | 0.75 | 7.23 ± 0.14 |
| DN-187 | 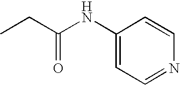 | >98 | 0.47 | 8.31 ± 0.07 |
| DN-189 | 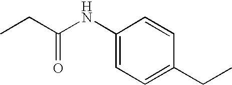 | >98 | 0.81 | 5.87 ± 0.17 |
| DN-199 | 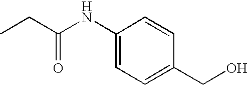 | >98 | 0.81 | 10.6 ± 3.0 |
| DN-209 | 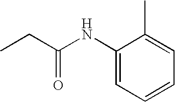 | >98 | 0.96 | 24.5 ± 1.0 |
| DN-210 | 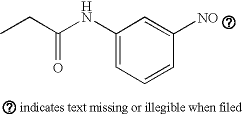 | >98 | 0.73 | 7.12 ± 1.3 |
| DN-213 | 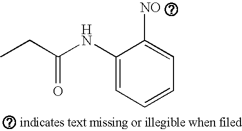 | >98 | 0.80 | 17.8 ± 4.0 |
| DN-218 | 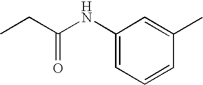 | >98 | 0.84 | 6.59 ± 0.89 |
| DN-222 | 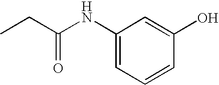 | >98 | 0.81 | 9.82 ± 1.92 |
| DN-229 | 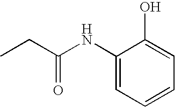 | >98 | 0.82 | 9.06 ± 0.41 |
| DN-231 | 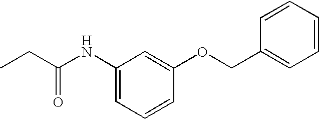 | >98 | 0.97 | 14.3 ± 0.25 |
| DN-234 | 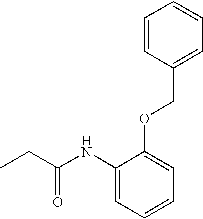 | >98 | 0.94 | 8.87 ± 1.09 |
| DN-238 | 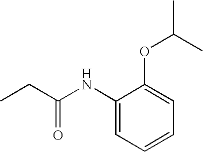 | >98 | 0.86 | 12.9 ± 1.4 |
| DN-242 | 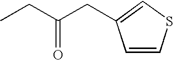 | >98 | 0.83 | 7.40 ± 1.04 |
| DN-248 | 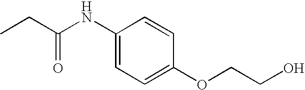 | >98 | 0.74 | 16.4 ± 0.7 |
| DN-260 | 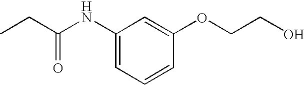 | >98 | 0.80 | 14.9 ± 0.4 |
| DN-261 | 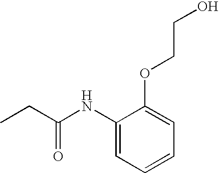 | >98 | 0.82 | 17.6 ± 1.7 |
| DN-263 | 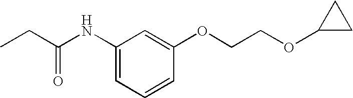 | >98 | 0.83 | 36.2 ± 6.5 |
| DN-265 | 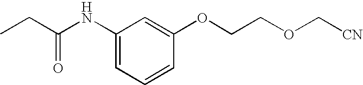 | >98 | 0.87 | 22.0 ± 0.4 |
| DN-268 | 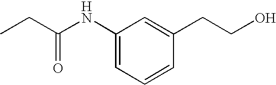 | >98 | 0.86 | 11.4 ± 0.1 |
| DN-271 | 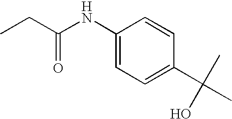 | >98 | 0.88 | 10.9 ± 1.3 |

TABLE 2
SAR analysis.

Chemical structures of the cysteine modifying compounds and IC₅₀
values of the correspondingly derivatized D1D2 to the binding of
D1D2 and YU2 gp120. The IC50 values are presented as the mean ±
SD values from two to three independent experiments.

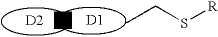

TABLE 2.1
R as N-alkyl-acetamide.

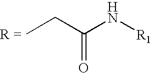

| | | IC₅₀ ± SD of |
|---|---|---|
| | | Derivatized |
| Compound | R₁ | D1D2 (nM) |
| Iodoacetamide | H | 33.3 ± 5.5 |
| 1 | 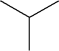 | 184 ± 39 |
| 2 | 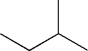 | 29.4 ± 2.2 |
| 9 | 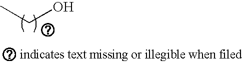 | 33.0 ± 9.7 |
| 3 | 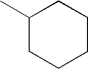 | 13.0 ± 2.1 |
| 4 | 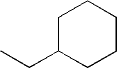 | 7.79 ± 2.08 |
| 5 | 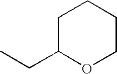 | 17.0 ± 2.5 |
| 6 | 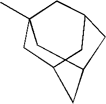 | 223 ± 49 |
| DN-040 | 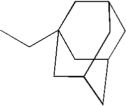 | 42.3 ± 5.9 |
| 8 | 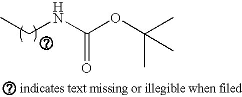 | 1.68 × 10³ ± 0.50 × 10³ |

TABLE 2.2
R as N-aryl-acetamide.

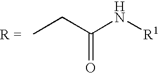

| | | IC₅₀ ± SD |
|---|---|---|
| | | of |
| | | Derivatize |
| Compound | R₁ | [symbol] |
| 10 | 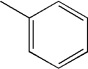 | 7.76 ± 0.83 |
| 28 | 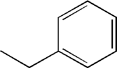 | 15.2 ± 0.7 |
| 29 | 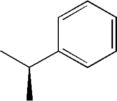 | 88.7 ± 1.5 |
| 30 | 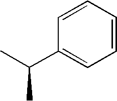 | 166 ± 22 |
| 40 | 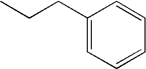 | 10.6 ± 2.9 |
| DN-022 | 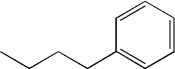 | 16.4 ± 2.6 |
| 14 | 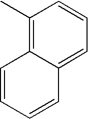 | 10.4 ± 0.8 |
| 32 | 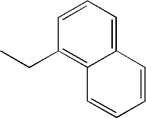 | 75.7 ± 11.2 |
| DN-149 | 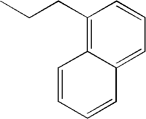 | 69.3 ± 1.8 |
| 31 | 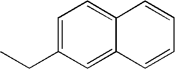 | 12.2 ± 0.8 |
| DN-152 | 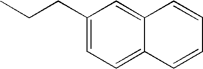 | 146 ± 4 |

[symbol]

indicates data missing or illegible when filed

TABLE 2.3
R as N-thiophen (furan)-acetamide

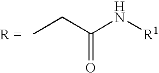

| | | IC₅₀ ± SD of |
|---|---|---|
| | | Derivatized |
| Compound | R₁ | D1D2 (nM) |
| DN-242 | 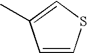 | 7.40 ± 1.04 |
| DN-155 | 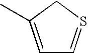 | 18.2 ± 1.8 |
| DN-170 | 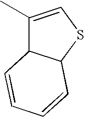 | 13.1 ± 0.5 |
| DN-028 | 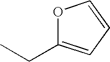 | 18.3 ± 2.1 |
| DN-034 | 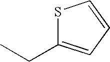 | 18.8 ± 0.5 |

TABLE 2.4
R as substituted N-phenyl-acetamide.

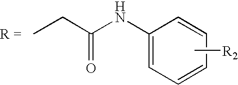

| | | |
|---|---|---|
| | | IC₅₀ ± SD of |
| | | Derivatized |
| Compound | R₂ | D1D2 (nM) |

A) para subsituents:

| | | |
|---|---|---|
| DN-183 | — | 7.07 ± 1.51 |
| DN-060 | OH | 11.4 ± 0.2 |
| DN-012 | —F | 7.08 ± 1.24 |
| DN-189 | 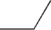 | 5.87 ± 0.17 |
| DN-199 | 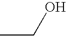 | 10.6 ± 3.0 |
| 12 | 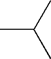 | 4.84 ± 0.83 |
| 13 | 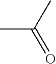 | 8.14 ± 0.38 |
| DN-052 | —NO₂ | 4.14 ± 0.03 |
| DN-030 | 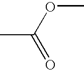 | 9.87 ± 0.67 |
| DN-271 | 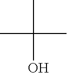 | 10.9 ± 1.3 |
| DN-185 | 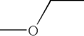 | 7.23 ± 0.14 |
| 22 | 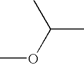 | 1.50 × 10³ ± 0.33 × 10³ |
| DN-248 | 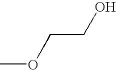 | 16.4 ± 0.7 |
| 27 | 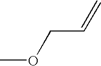 | 1.21 × 10³ ± 0.34 × 10³ |
| 23 | 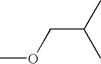 | 637 ± 202 |
| 17 | 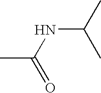 | 402 ± 68 |
| 16 | 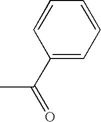 | 46.2 ± 1.3 |
| 26 | 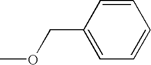 | 290 ± 84 |

B) meta subsituents:

| | | |
|---|---|---|
| DN-218 | — | 6.59 ± 0.89 |
| DN-222 | —OH | 9.82 ± 1.92 |
| DN-210 | —NO₂ | 7.12 ± 1.3 |
| DN-268 | 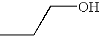 | 11.4 ± 0.1 |
| DN-260 | 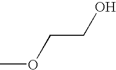 | 14.9 ± 0.4 |
| DN-265 | 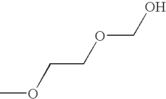 | 22.0 ± 0.4 |
| DN-231 | 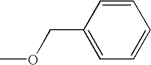 | 14.3 ± 0.25 |
| DN-263 | 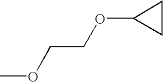 | 36.2 ± 6.5 |

C) ortho subsituents:

| | | |
|---|---|---|
| DN-209 | — | 24.5 ± 1.0 |
| DN-229 | —OH | 9.06 ± 0.41 |
| DN-213 | —NO₂ | 17.8 ± 4.0 |
| DN-238 | 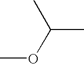 | 12.9 ± 1.4 |
| DN-261 | 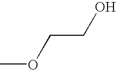 | 17.6 ± 1.7 |
| 15 | 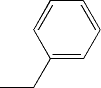 | 51.9 ± 6.3 |
| DN-234 | 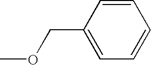 | 8.87 ± 1.09 |

TABLE 2.5
Role of acetamide moiety.

| | | IC₅₀ ± SD of |
|---|---|---|
| | | Derivatized |
| Compound | R | D1D2 (nM) |
| 10 | 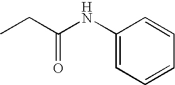 | 7.76 ± 0.83 |
| DN-146 | 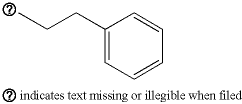 | 24.7 ± 8.6 |
| DN-180 | 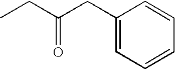 | 26.4 ± 3.9 |

TABLE 2.6
Mixed-disulfide compounds derivatized D1D2F43C

| | | IC₅₀ ± SD of |
|---|---|---|
| | | Derivatized |
| Compound | R | D1D2 (nM) |
| 42 | 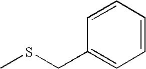 | 11.5 ± 1.8 |
| 43 | 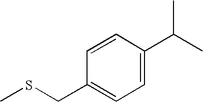 | 9.14 ± 1.99 |
| 44 | 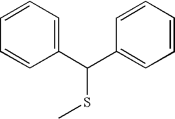 | 1.38 × 10³ ± 0.10 × 10³ |
| 45 | 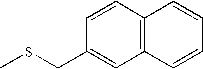 | 14.3 ± 0.6 |
| 47 | 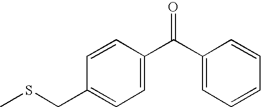 | 27.0 ± 1.6 |
| 48 | 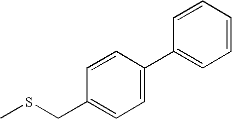 | 14.9 ± 0.0 |
| DN-146 | 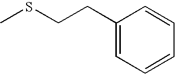 | 24.7 ± 8.6 |

TABLE 2.7
Affinities and structures of 14 new modified D1D2F43C.

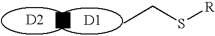

| | | IC₅₀ of |
|---|---|---|
| Compound | R | Derivatized |
| DN-2-56 | 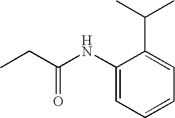 | 39.6 |
| DN-2-62 | 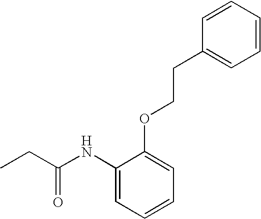 | 46.4 |
| DN-2-63 | 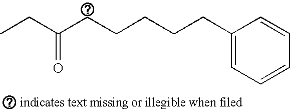 | 29.0 |
| DN-2-64 | 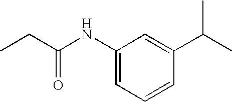 | 9.1 |
| DN-2-70 | 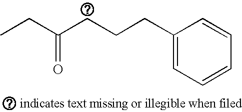 | 174 |
| DN-2-75 | 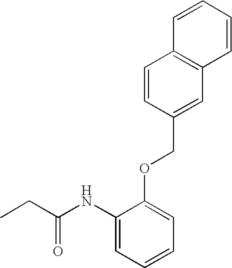 | 135 |
| DN-2-76 | 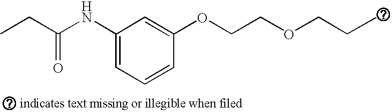 | 216 |
| DN-2-78 | 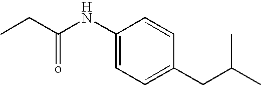 | 14.3 |
| DN-2-201 | 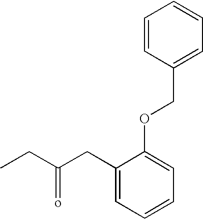 | 23.7 |
| JRC-1-14 | 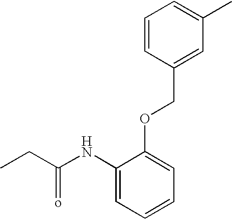 | 25.7 |
| JRC-1-114 | 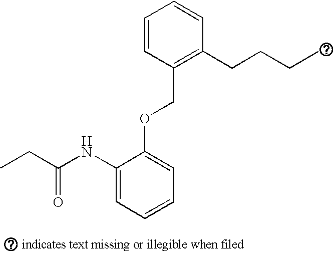 | 264 |
| JMC-1-38 | 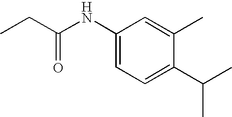 | 6.4 |
| JMC-1-66 | 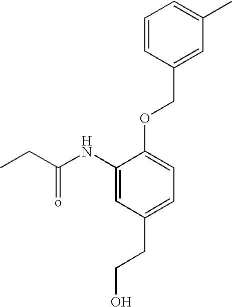 | 678 |
| JMC-1-81 | 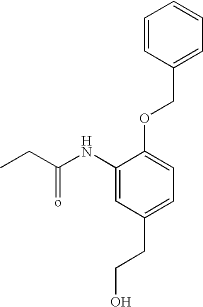 | 305 |

Note:
Binding data shown for each compound were obtained using D1D2 derivative for such compound, per methods described for Table 2.

### Example III

### Derivatized CD4 Molecules as Improved Therapeutic Agents

The method that we have described for identifying chemical leads for inhibition of the gp120-CD4 interaction has already produced several derivatized CD4 analogs that bind to HIV gp120 as well or better than natural human CD4. Various constructs based on human CD4 have been shown to be efficacious (e.g. CD4-IgG2 and dodecameric CD4-Ig), even in clinical trials, and it can be expected that such constructs modified to incorporate the very same modifications that we have introduced by reacting D1D2F43C with certain of our bromoacetamides or 5-nitro-2-pyridyldisulfides (e.g. SNS-10, SNS-12, SNS-14, DN-52 and DN-234) might themselves be expected to have better therapeutic efficacy than the parent therapeutic CD4 in treating neonates of HIV-infected mothers and newly HIV-infected medical workers (needle pricks).

The incorporation of our derivatives into an already developed CD4 product would require the following additional steps: 1. The recombinant cDNA used to produce the therapeutic CD4 product would need to be modified to encode the F43C mutation. 2. The alternative therapeutic CD4F43C would need to be reacted according to the same procedures used with D1D2F43C for modification with our derivatizing reagent to produce the product analogous to our D1D2F43C-X derivative.

The Phe43 interaction is the focal point of all CD4-gp120 interactions, and we expect that properties observed for derivatized D1D2F43C will transfer faithfully to any CD4-based therapeutic. Based on our analysis of derivatives of D1D2F43C, derivatized analogues of current therapeutic CD4s can be expected to have two advantages over the current therapeutics. First, binding affinities have been found that are higher than that of wild-type CD4 (e.g. D1D2F43C-DN-52 has a measured affinity 77% greater than that of natural D1D2). Structure-based design efforts that are in progress may lead to further improvements. Increased affinity is advantageous since it increased the potency of the drug. Second, larger derivatives are found to cause structural perturbations in gp120 and to lead to decreased affinity for the 17b antibody, a surrogate for the chemokine receptor, and to an observed decrease in binding of YU2 gp120 to the CCR5 chemokine receptor. Decreased affinity for the chemokine receptor is advantageous since it reduces the risk of adventious viral virion after association with the therapeutic CD4. A risk to be contemplated for these derivatives is that of a potential immune response directed against the new chemical moiety on CD4.

### REFERENCES

Allaway, G. P., K. L. Davis-Bruno, G. A. Beaudry, E. B. Garcia, E. L. Wong, A. M. Ryder, K. W. Hasel, M. C. Gauduin, R. A. Koup, J. S. McDougal and et al. (1995). “Expression and characterization of CD4-IgG2, a novel heterotetramer that neutralizes primary HIV type 1 isolates.” AIDS Res Hum Retroviruses 11(5): 533-9.
Gauduin, M. C., G. P. Allaway, P. J. Maddon, C. F. Barbas, 3rd, D. R. Burton and R. A. Koup (1996). “Effective ex vivo neutralization of human immunodeficiency virus type 1 in plasma by recombinant immunoglobulin molecules.” J Virol 70(4): 2586-92.
Trkola, A., A. B. Pomales, H. Yuan, B. Korber, P. J. Maddon, G. P. Allaway, H. Katinger, C. F. Barbas, 3rd, D. R. Burton, D. D. Ho and J. P. Moore (1995). “Cross-clade neutralization of primary isolates of human immunodeficiency virus type 1 by human monoclonal antibodies and tetrameric CD4-IgG.” J Virol 69(11): 6609-17.
Arthos, J., C. Cicala, T. D. Steenbeke, T. W. Chun, C. Dela Cruz, D. B. Hanback, P. Khazanie, D. Nam, P. Schuck, S. M. Selig, D. Van Ryk, M. A. Chaikin and A. S. Fauci (2002). “Biochemical and biological characterization of a dodecameric CD4-Ig fusion protein: implications for therapeutic and vaccine strategies.” J Biol Chem 277(13): 11456-64.

### Example IV

### Structure Studies on the Interaction of Chemically Derivatized CD4 with HIV gp120

### Introduction

The surfaces of HIV viruses contain noncovalent trimeric association of the envelope glycoproteins gp41 and gp120 (Clapham et al. 2002). The gp120 proteins mediate the initial attachment step in HIV viral entry into host cells by sequentially interacting with host cell receptor CD4 and a chemokine receptor, CCR5 or CXCR4. In addition, it helps the virus to escape the neutralization of host immune system by 1) heavy glycosylation, 2) shielding of the conserved epitopes by highly variable loops and 3) protection of its active conformations by imposing large unfavorable entropy penalty for their transition from the free form (Kwong et al. 1998; Wyatt et al. 1998; Kwong et al. 2002). Thus, gp120 is a prominent target for therapeutic intervention either by blocking its binding to CD4 or the co-receptor or by eliciting gp120-directed neutralizing antibodies. Precise and comprehensive information on the structures of gp120 and their stability and flexibility is indispensable to advancing the progress of either approach.

Unfortunately, trimeric gp120 and full length monomeric gp120 have so far eluded crystallographic study, possibly due to the specific immune system-eluding structural characteristics mentioned above. Most of the known structural information on gp120 has come from a few X-ray crystal structures of core gp120 protein in complex with D1D2 and a Fab fragment of a gp120 antibody (17B or X5) (Kwong et al. 1998; Kwong et al. 2000; Huang et al. 2005) as well as a relative low-resolution structure of an unliganded SIV core gp120 (Chen et al. 2005). The structures of core gp120 bound by both D1D2 and an antibody are believed to reflect the true character of CD4-bound gp120 because the following reasons: 1) the gp120-CD4 interaction revealed by the crystal structures is consistent with the critical residues identified in both components by the mutational analysis (Kwong et al. 1998); and 2) core gp120 has been shown to resemble full length gp120 both structurally and functionally (Binley et al. 1998; Rizzuto et al. 1998; Myszka et al. 2000).

On the other hand, the crystal structure of free SIV core gp120 may still have non-trivial differences from the real free conformation of HIV gp120, due to the following concerns. Because of the flexible and partially-unfolded nature of free gp120 without its binding partners, a crystal structure can only provide a snapshot of one of its quasistates, which can be stabilized by crystal contacts. Oligomeric organization of gp120 may also provide additional constraints on the conformations of free gp120. Furthermore, the epitope for an antibody b12 that recognizes free gp120 with very small entropy change (Kwong et al. 2002) and neutralizes HIV-1 viruses broadly was mapped to a surface that is continuous in the structure of CD4-bound gp120 (Pantophlet et al. 2003) but not in the recently solved structure of the free SIV gp120 (Chen et al. 2005). Taken together, these evidences suggest that the SIV structure may resemble one of many conformations that free gp120 can adopt and most likely it is different from the gp120 structure in trimeric form. The predominant conformations of free gp120 may resemble CD4-bound gp120 structures more than the SIV core gp120 structure, but with substantial variation. Thus structural understanding of the flexibility of free gp120 and gp120 bound by different ligands are necessary for designing therapeutic agents targeting gp120.

In Experiments I and II, we have described the construction of a library of derivatized CD4 proteins for screening their binding affinities to the Phe43 cavity and presented a full structure-activity analysis of binding of these diverse chemical entities to the Phe43 cavity. Significant plasticity of gp120 in binding the derivatized CD4 has been also suggested by the SAR study. Here, we are interested in gaining structural understanding on the binding of gp120 to CD4 derivatives in order to aid further structure-based improvement of CD4-gp120 interaction inhibitors and also lend more insight into the plasticity and adaptability of gp120 in binding its ligands. Specifically, we solved crystal structures of core gp120 in complex with four differently derivatized CD4, each having high affinity in binding gp120. We describe here in detail how gp120 interacts with these CD4 derivatives and how these structures compare to a recently solved structure of gp120 binding to a CD4 mimetic, CD4M33 (Huang et al. 2005). Surprisingly large plasticity of gp120 in binding different derivatives was observed despite the fact CD4 has already constrained gp120 in a CD4-bound conformation. In addition, the thermodynamics of full-length gp120 binding to derivatized CD4 and 17b antibody was studied using isothermal titration calorimetric experiments. Reduced negative entropy and heat capacity changes in binding of gp120 and derivatized CD4 suggested for a less structured gp120 with Phe43-cavity occupied, when compared to CD4-bound gp120. This intermediate conformational state of gp120 may have reduced affinity for chemokine receptor, as suggested by an in vitro CCR5-binding experiment. A mechanism is proposed based on the crystal structures for the transduction of the conformational changes from the gp120 structure around the filled Phe43 cavity to farther regions in gp120 including chemokine-receptor binding site.

### Results

Overview of Structures of Four Derivatized CD4 in Complex with gp120

To study the structural bases for recognition of different D1D2F43C derivatives by gp120, we crystallized and solved structures of four D1D2F43C derivatives in complexes with HXBc2 core gp120 and Fab fragment of 17b (FIG. 21). These four CD4 derivatives were selected because of their structurally diverse modifications on the Cys43 and their high affinities for gp120 (IC₅₀ 7-10 nM, similar as D1D2). They were derived from modifications of D1D2F43C proteins by the following bromoacetamide compounds: SNS-10, SNS-14, SNS-40, and DN-234. The chemical groups attached to the common acetamide moiety that is linked to Cys43 in D1D2 are phenyl, phenethyl, naphthyl and benzyloxy-phenyl groups respectively. The corresponding gp120 tertiary complexes gp120:17b:derivatized-D1D2 (“derivatized HX complexes”) were named HX-SNS-10, HX-SNS-14, HX-SNS-40, and HX-DN-234 respectively.

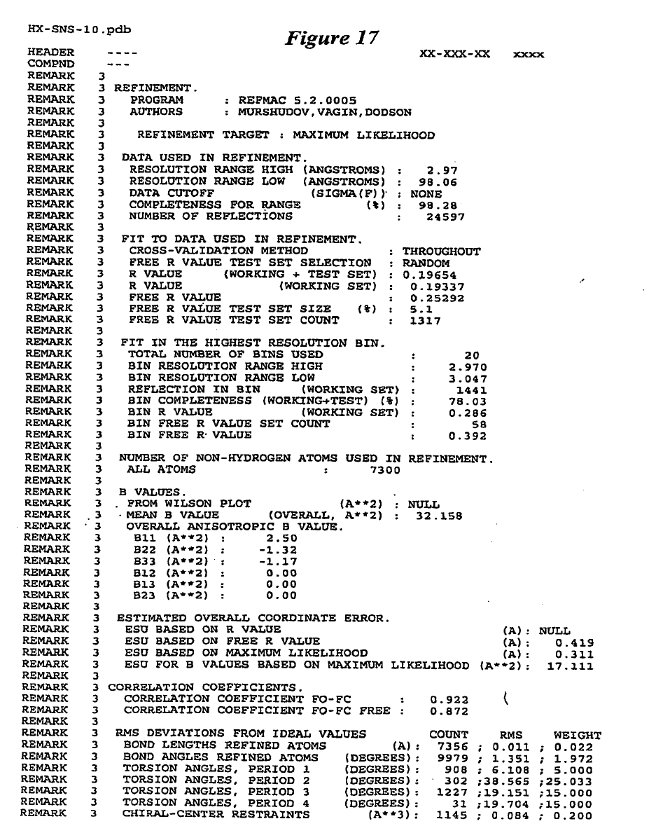

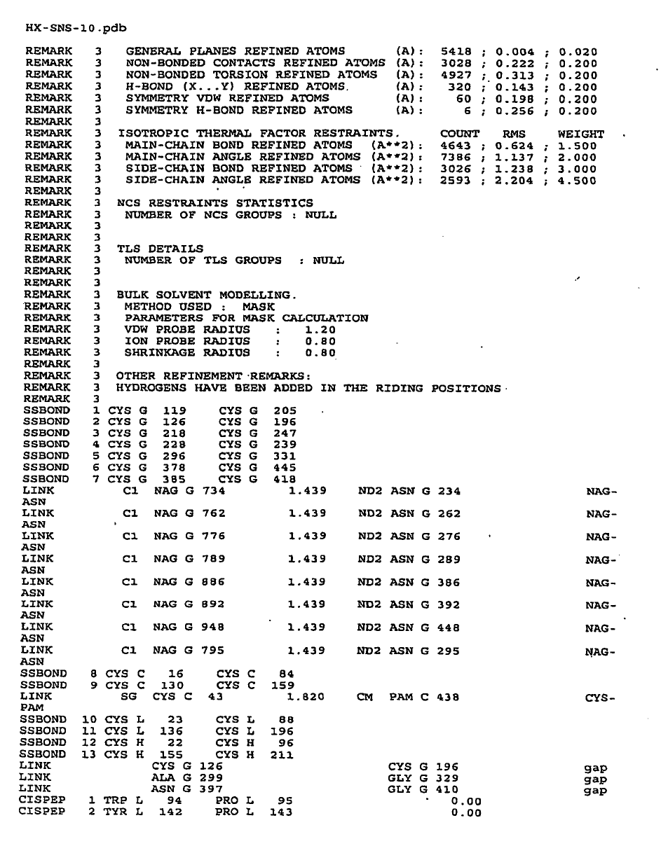

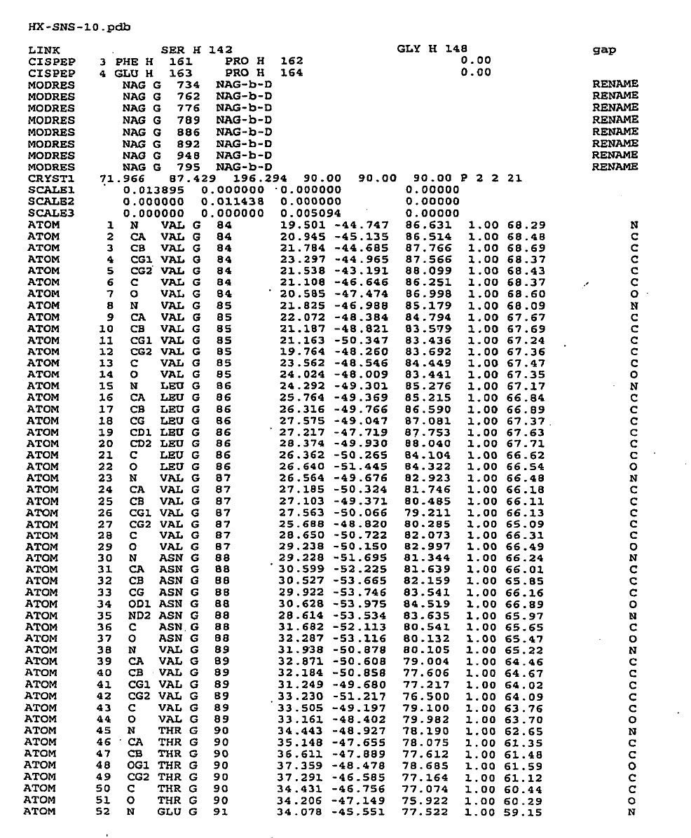

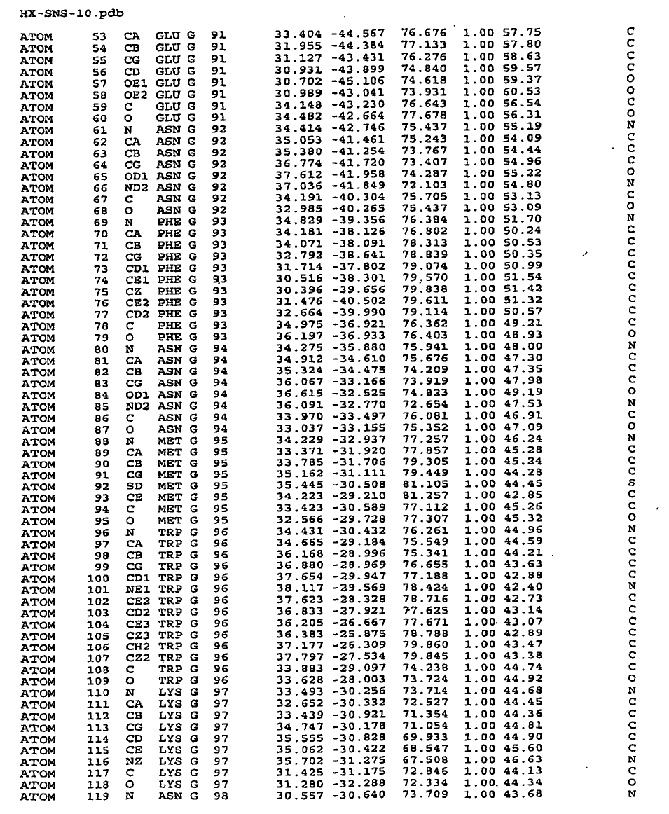

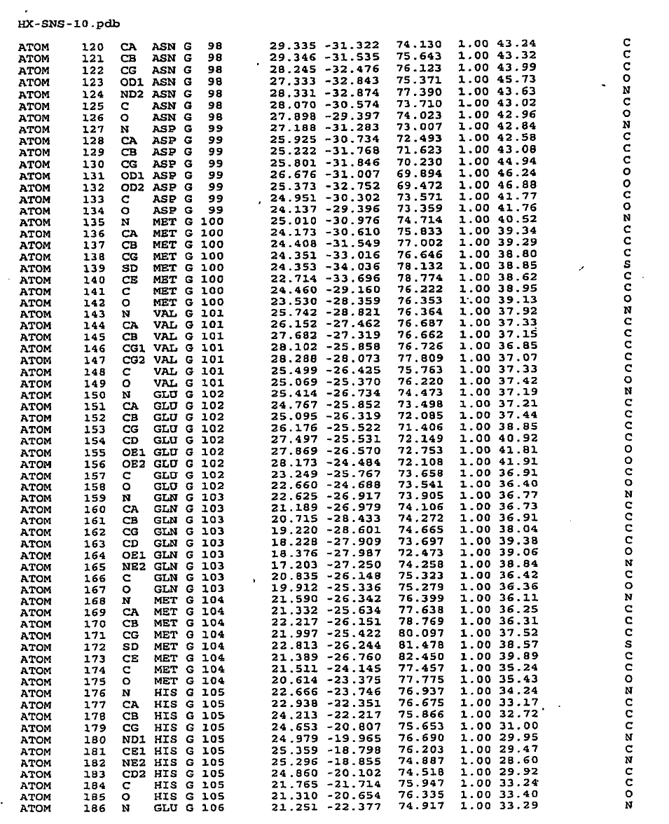

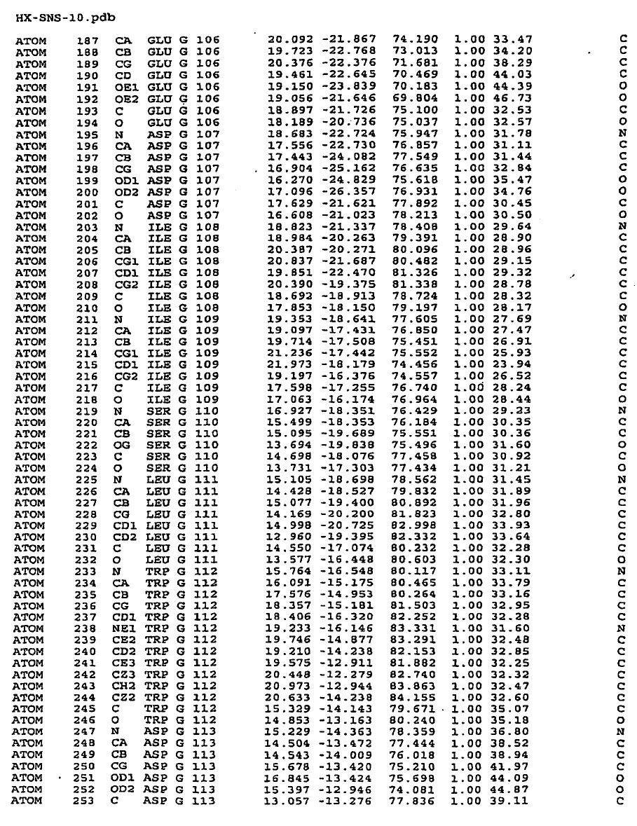

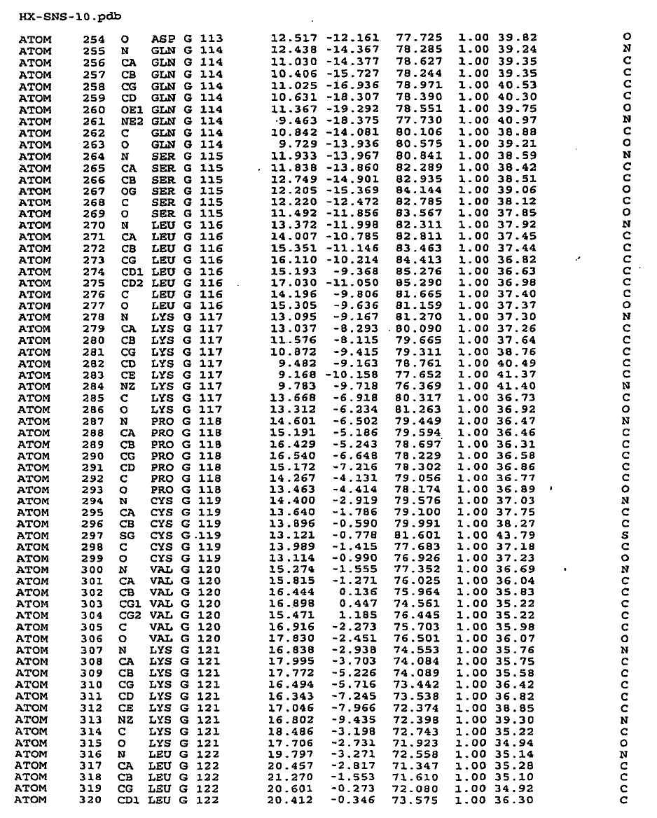

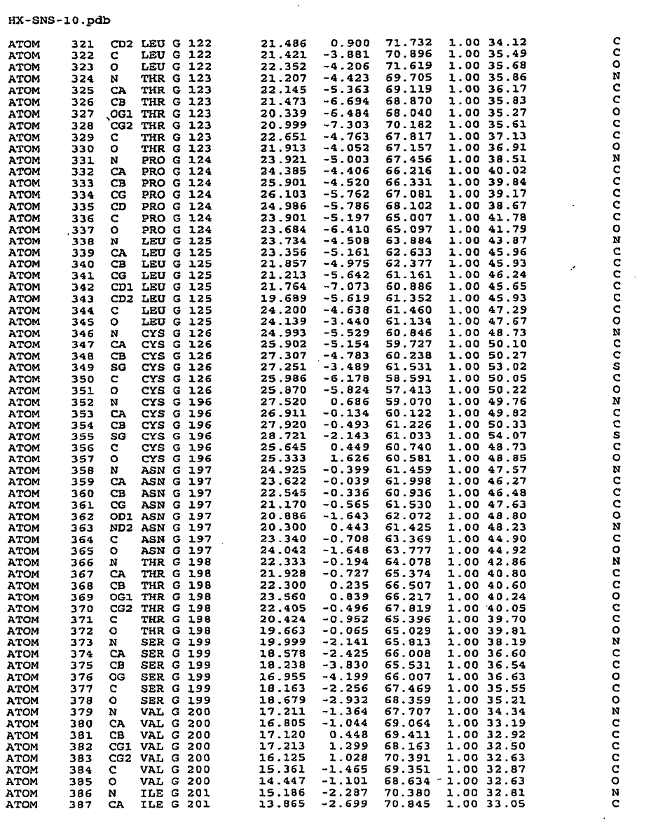

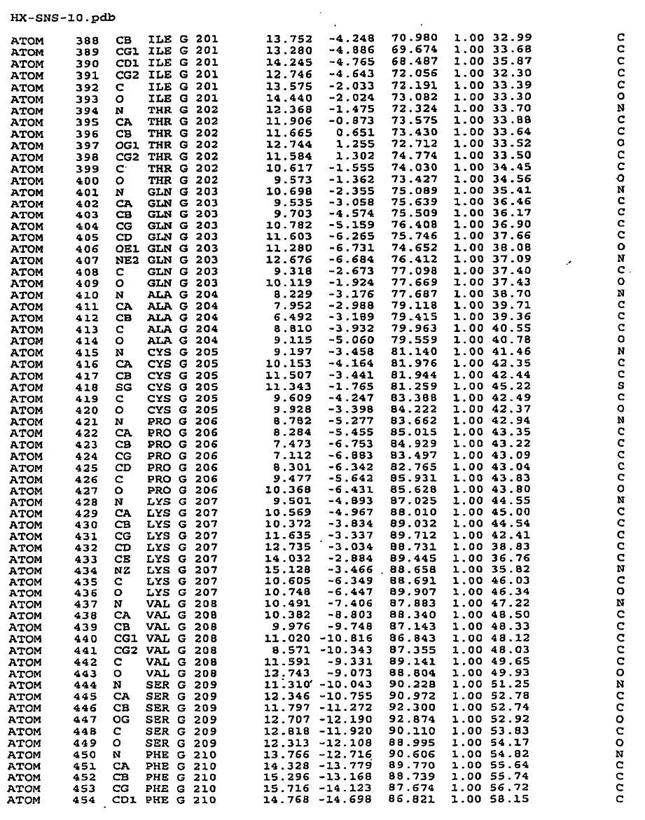

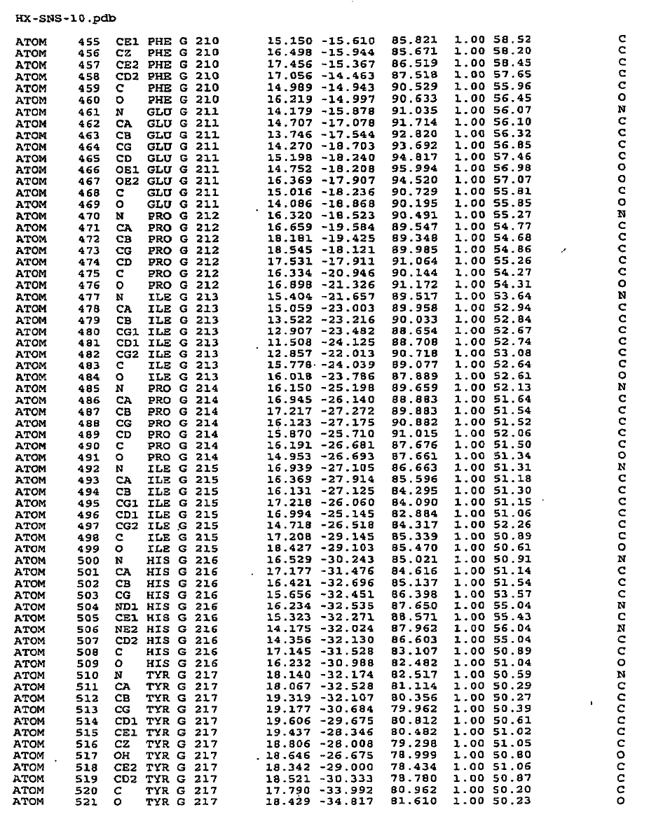

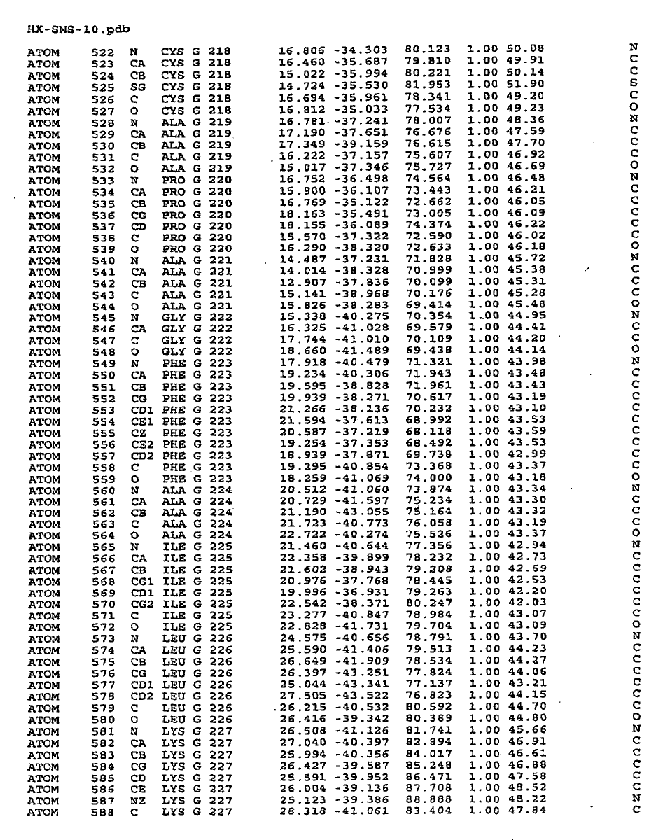

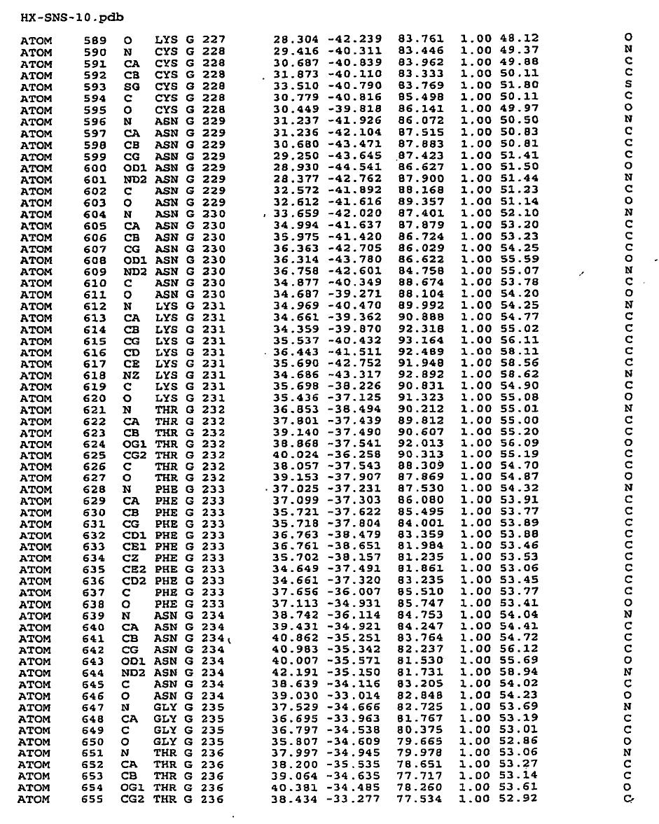

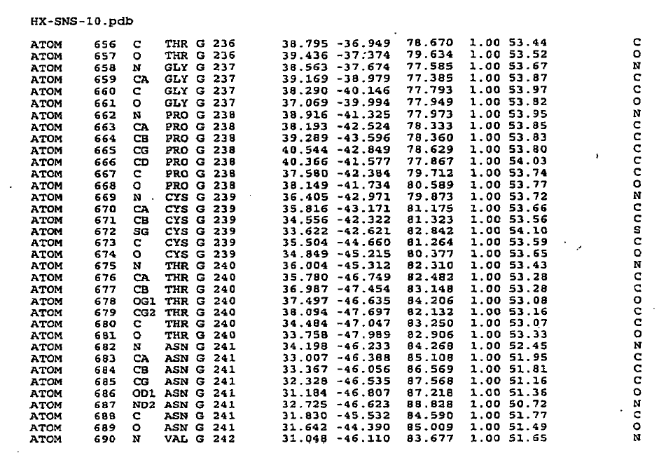

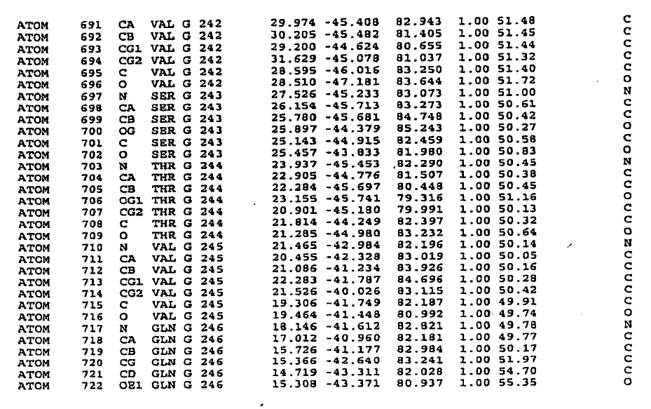

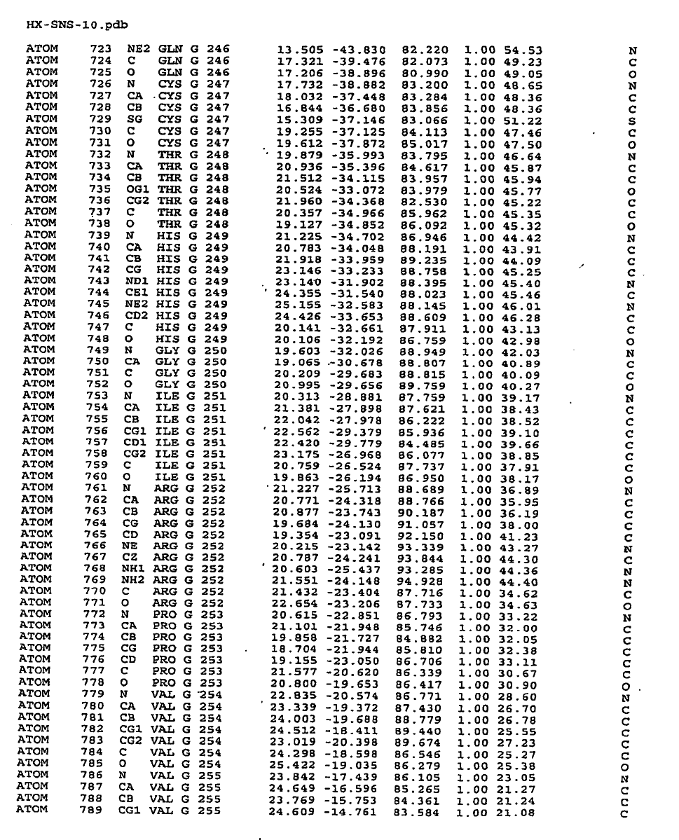

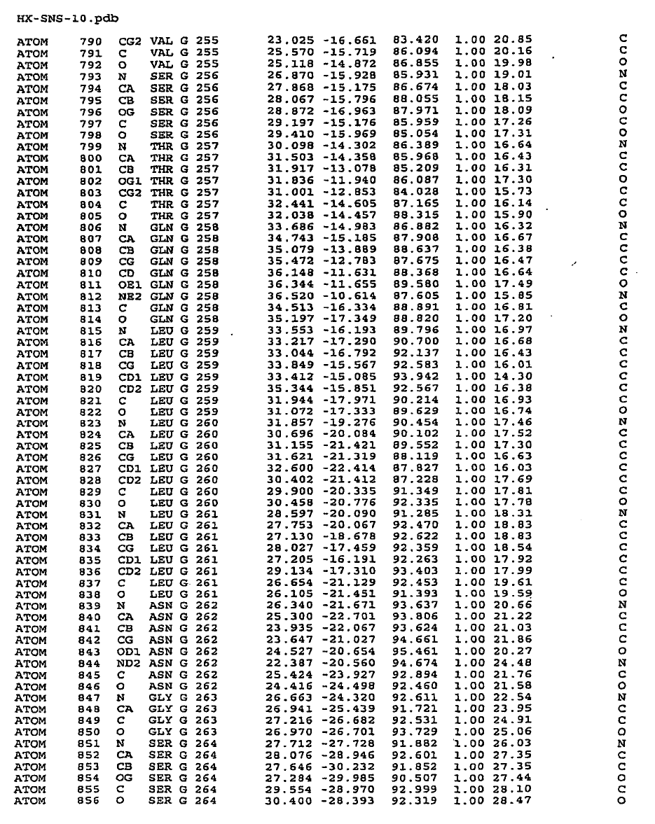

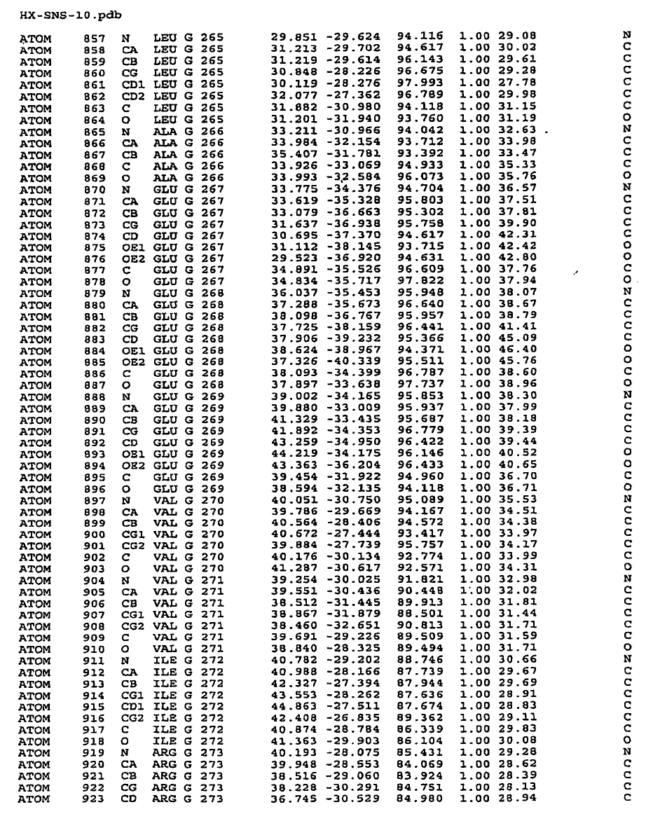

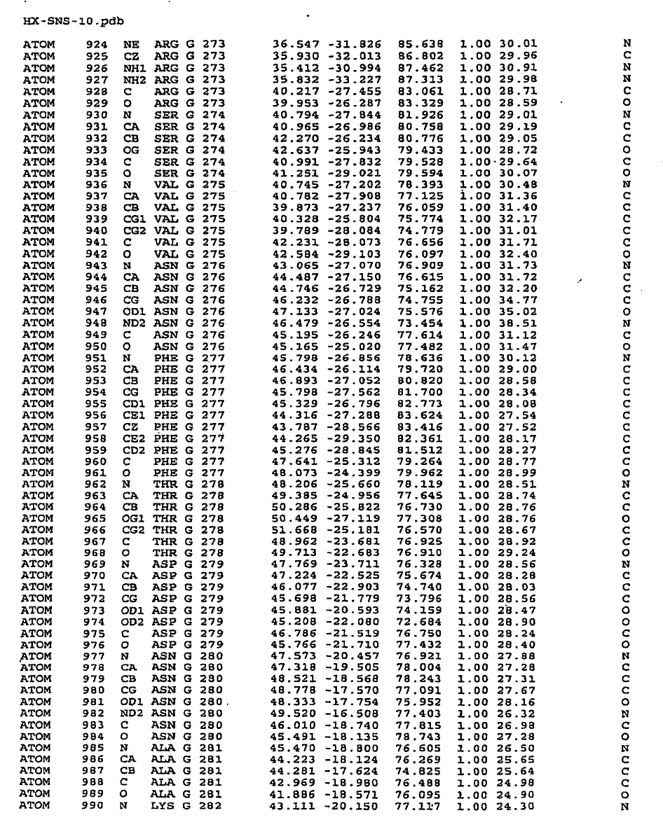

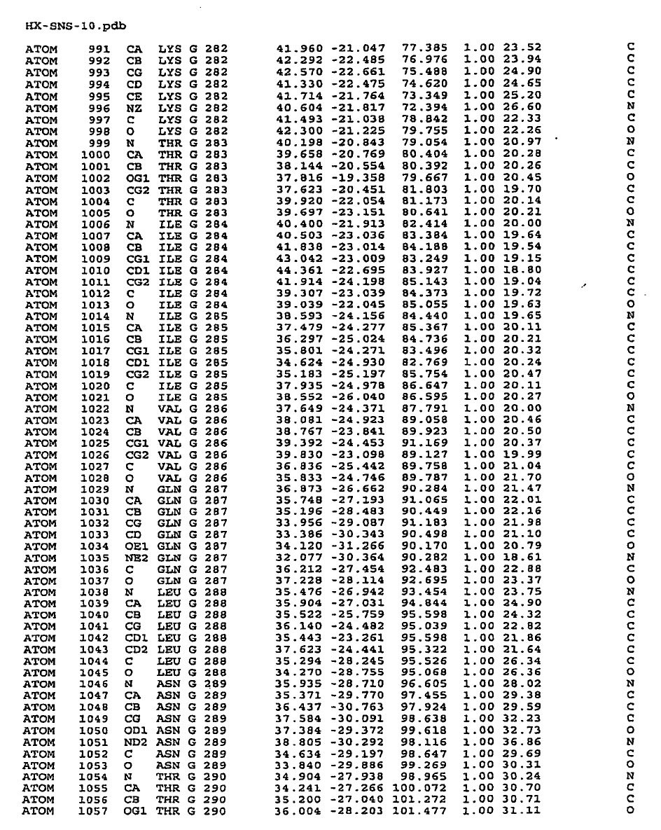

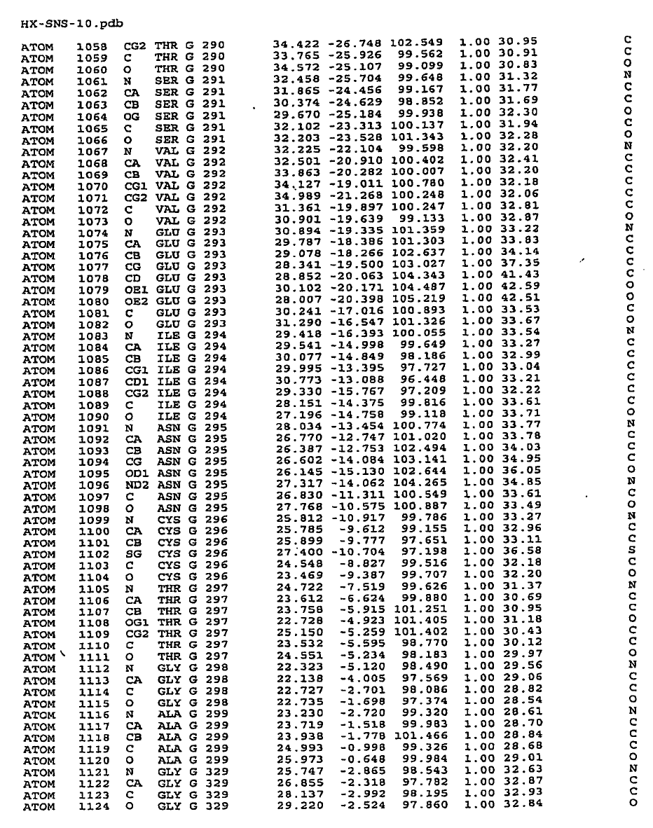

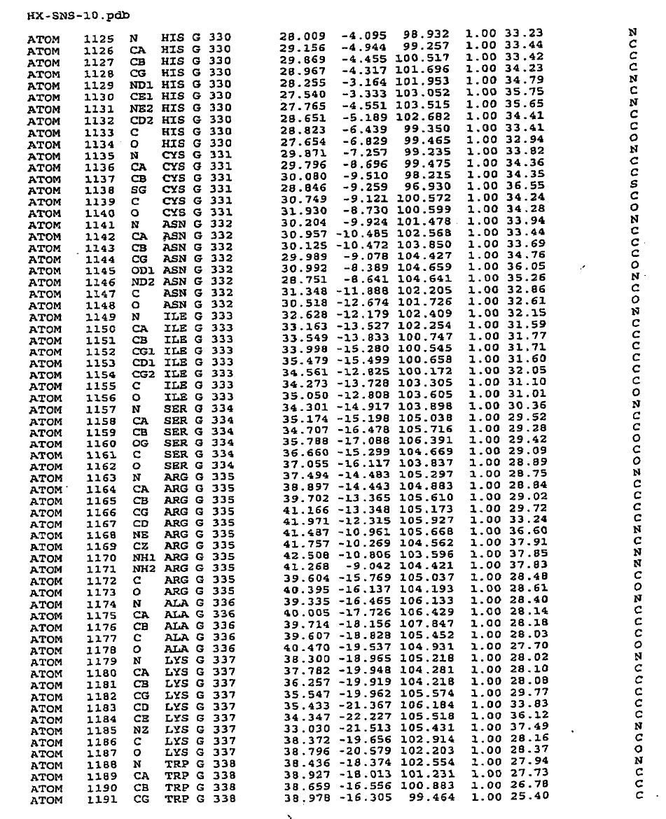

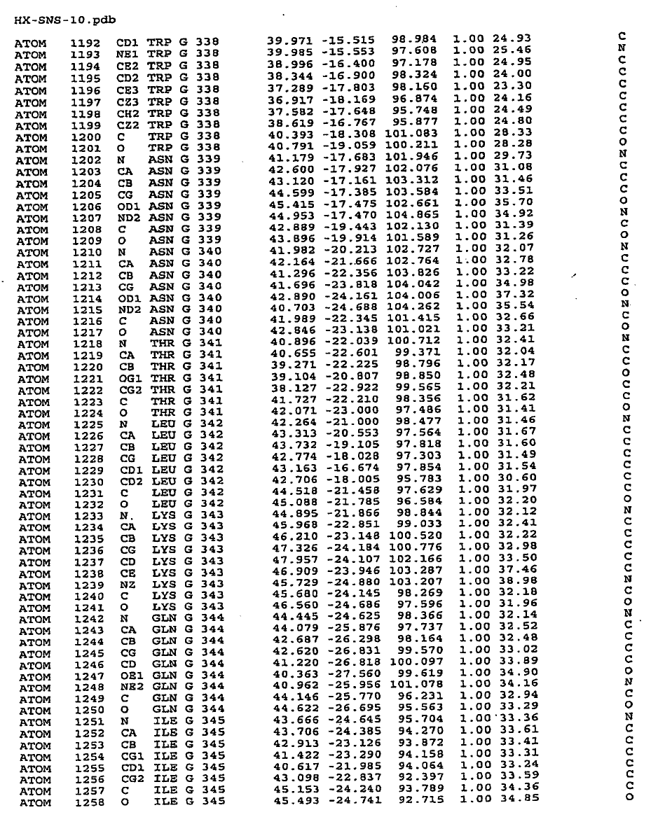

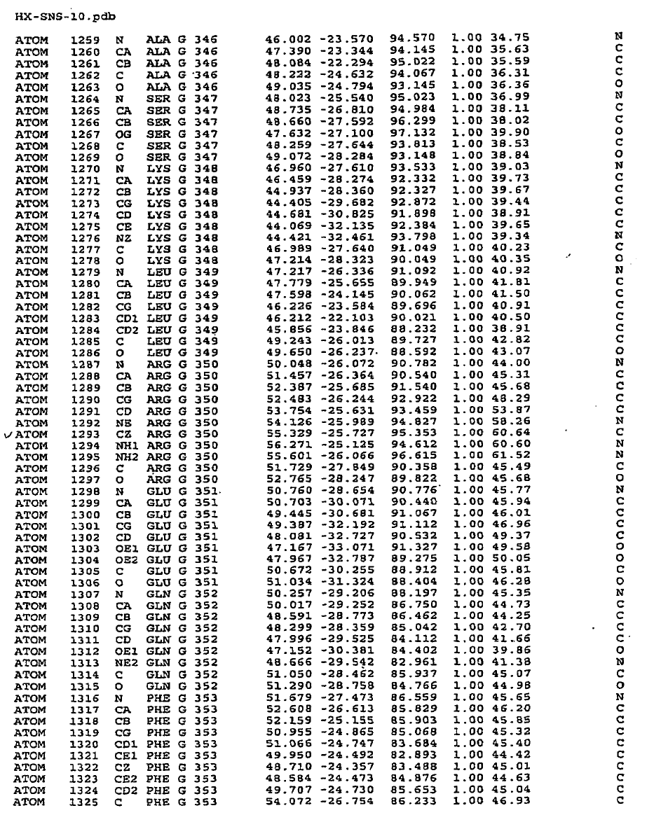

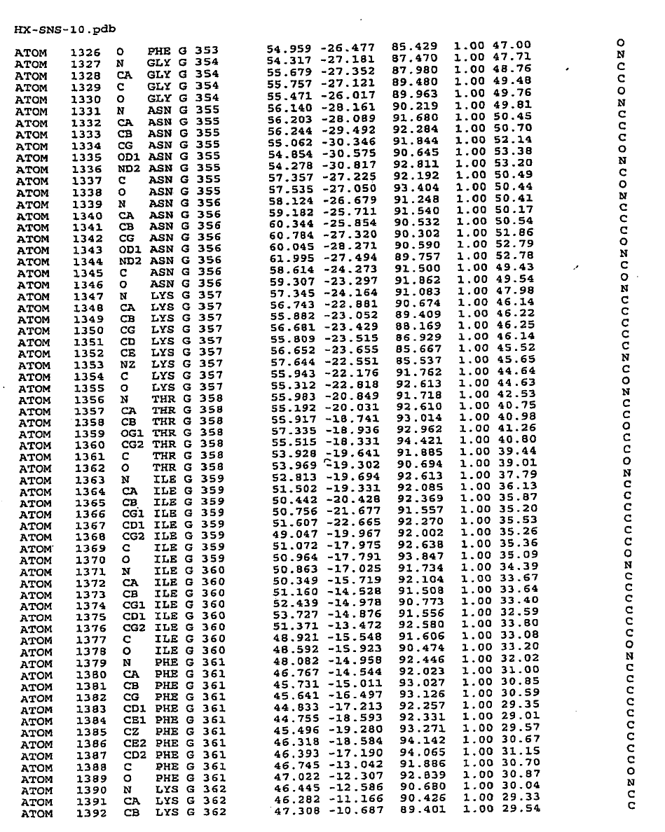

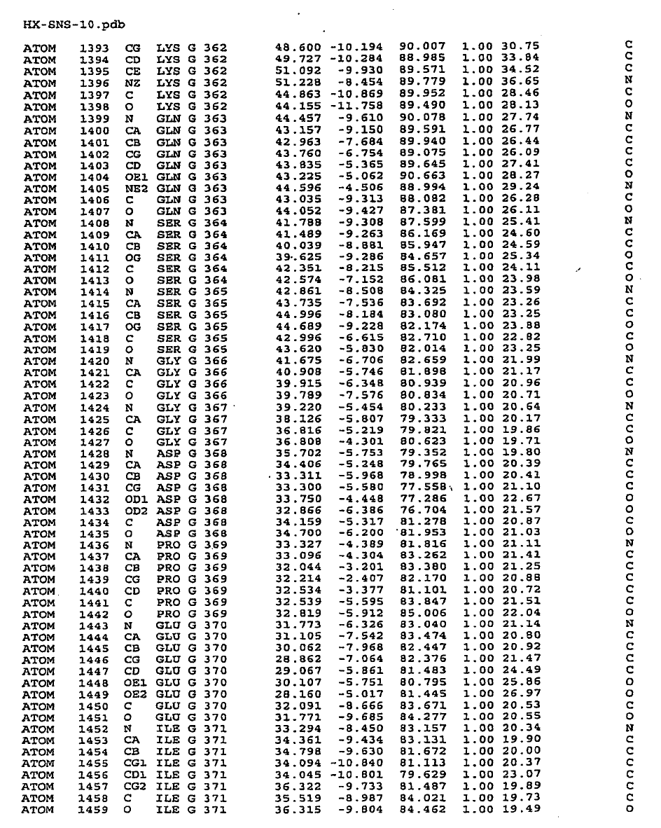

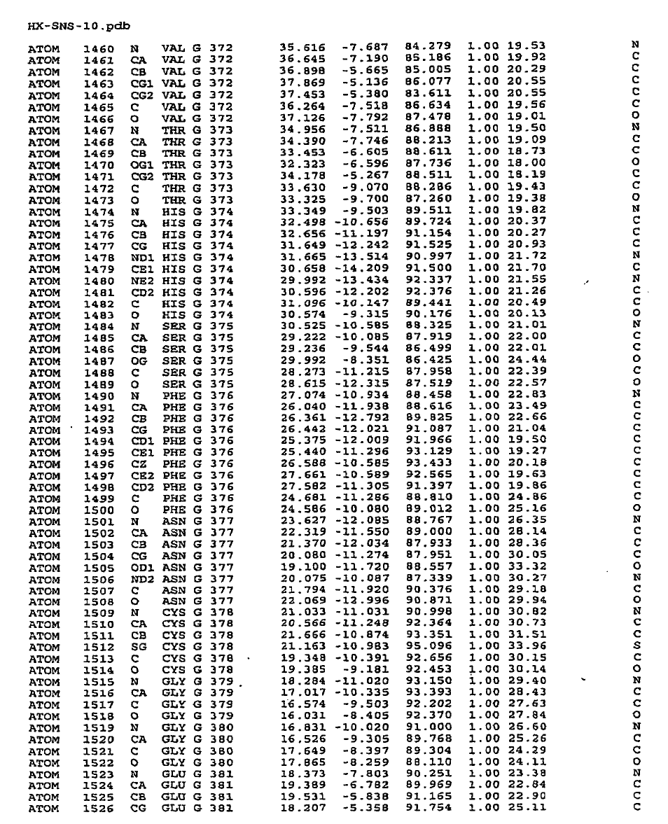

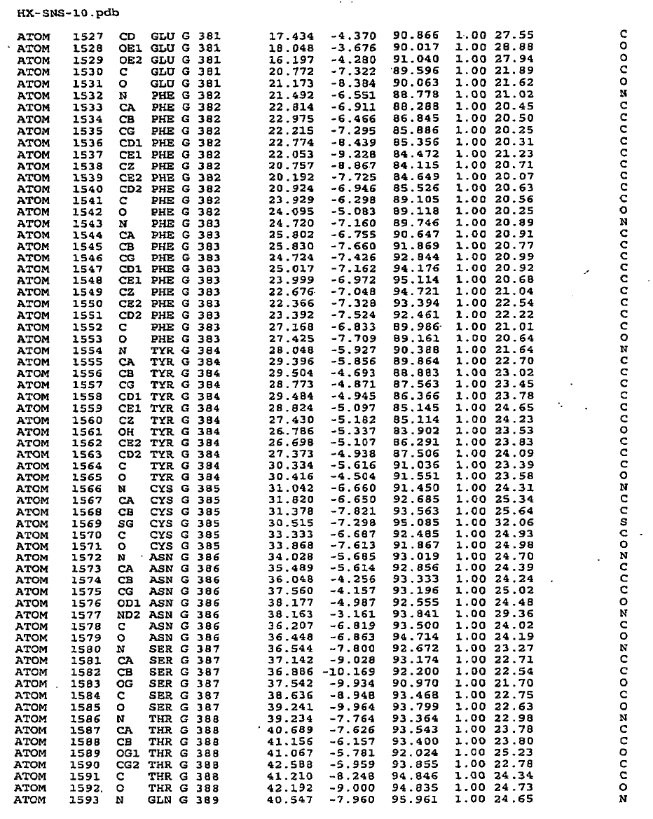

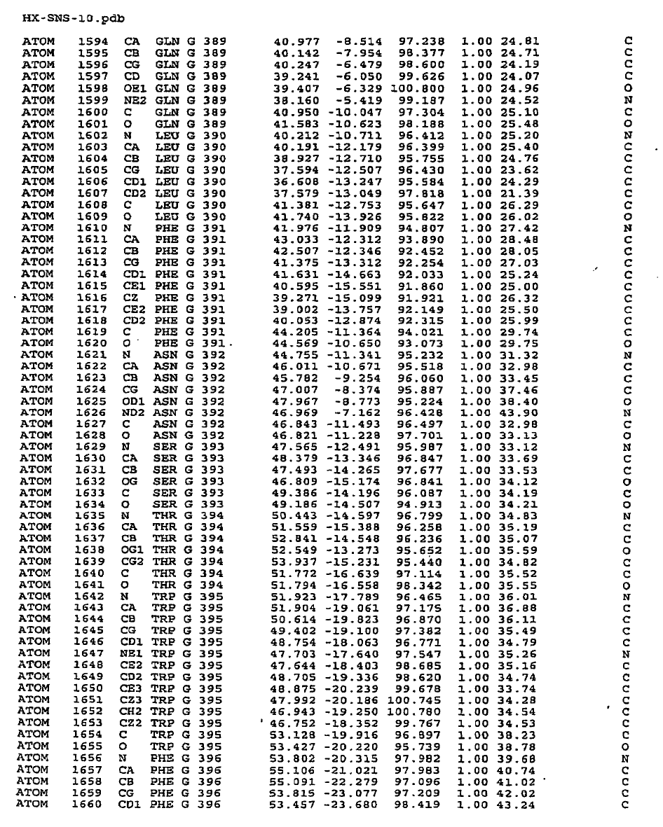

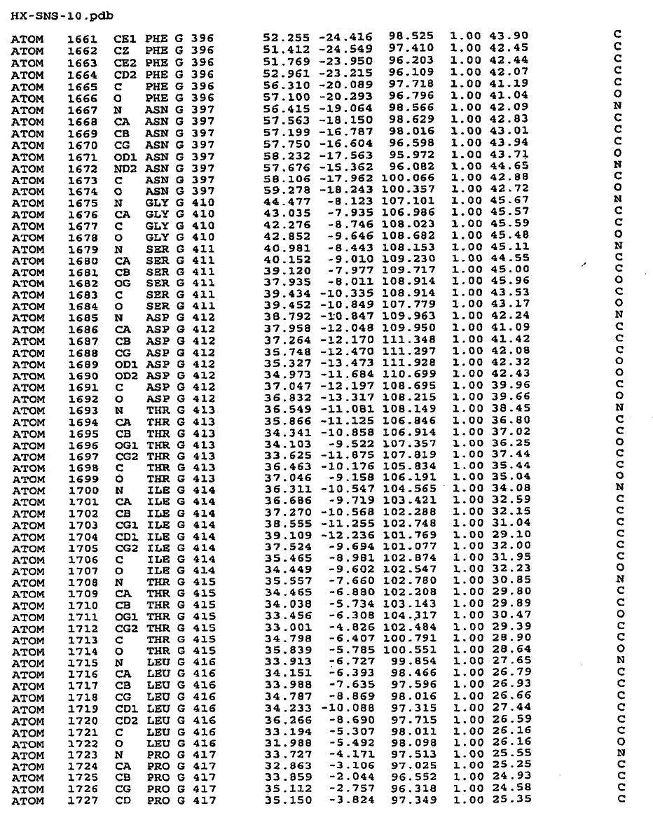

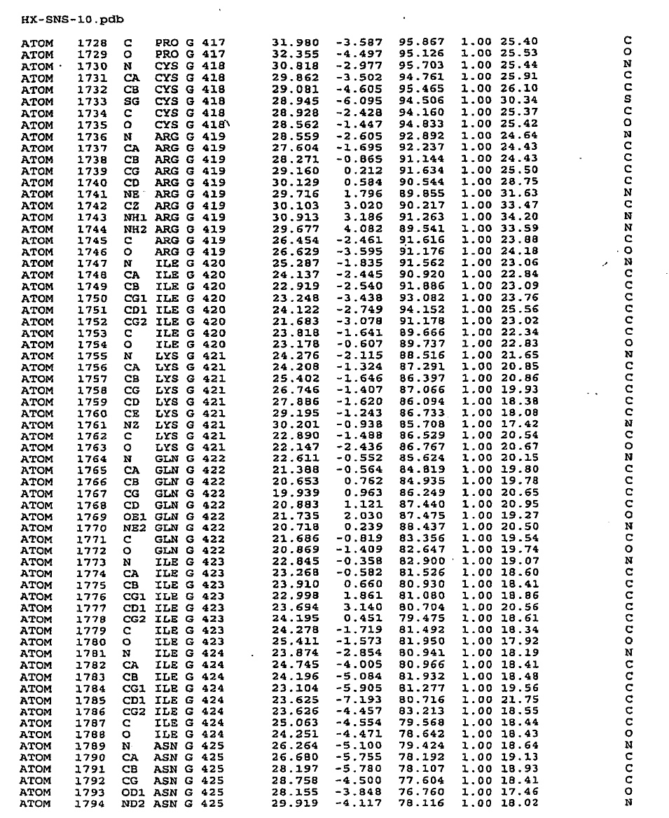

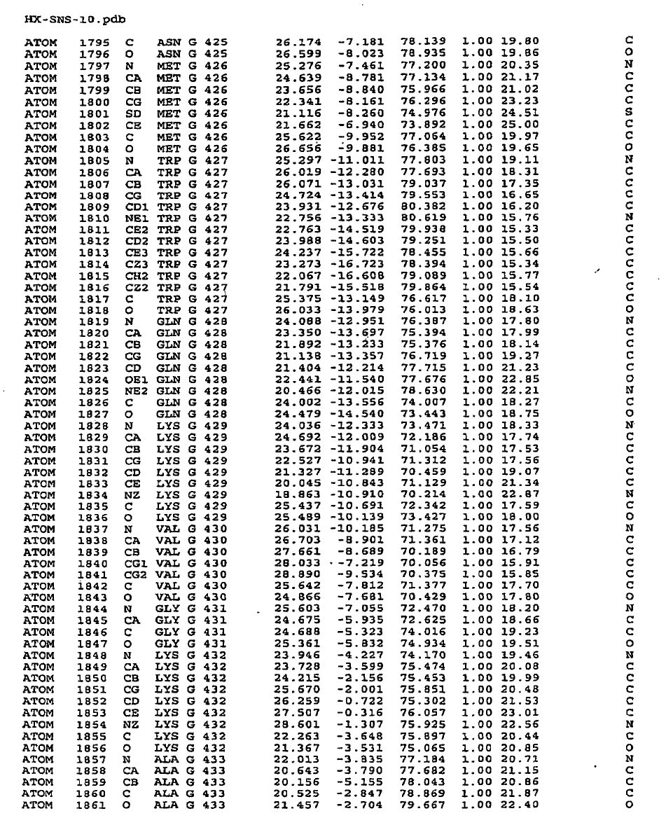

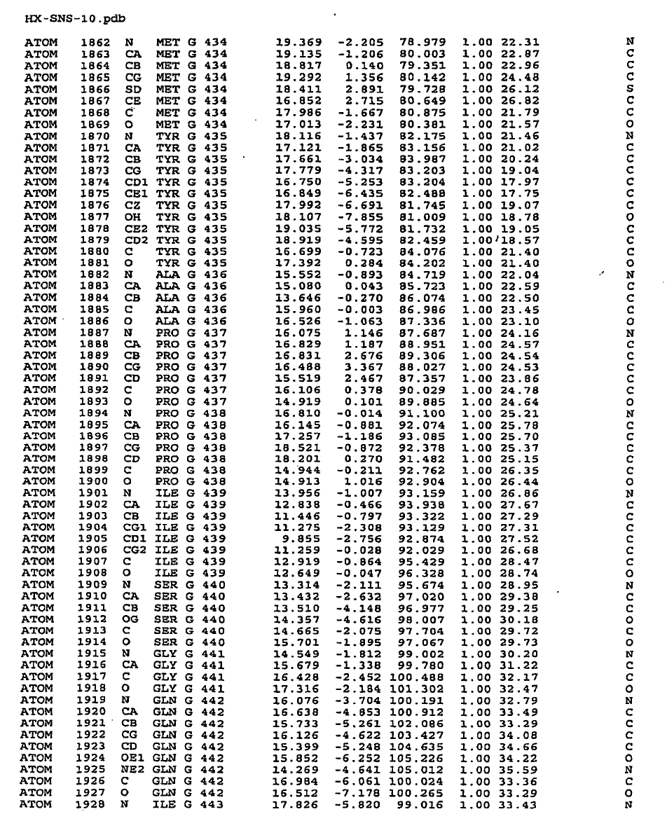

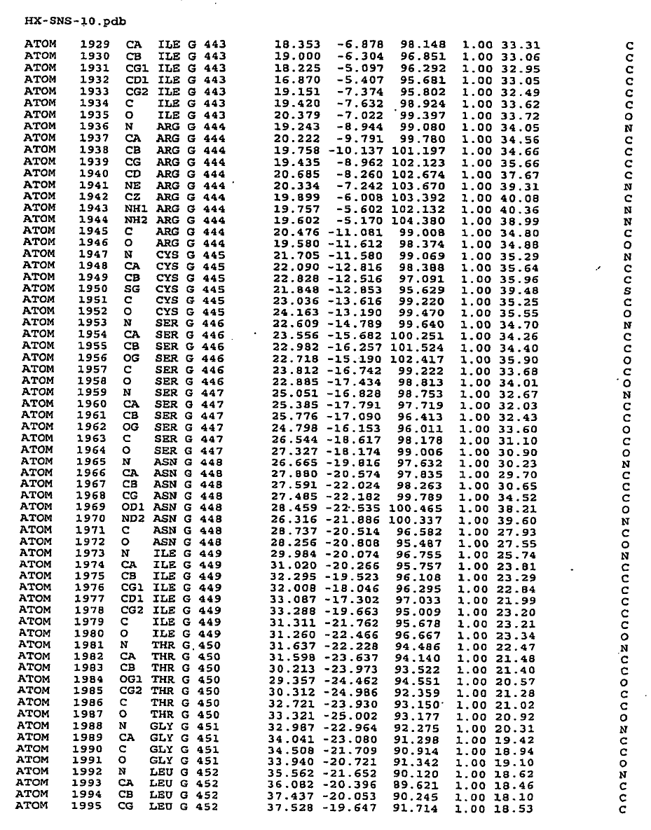

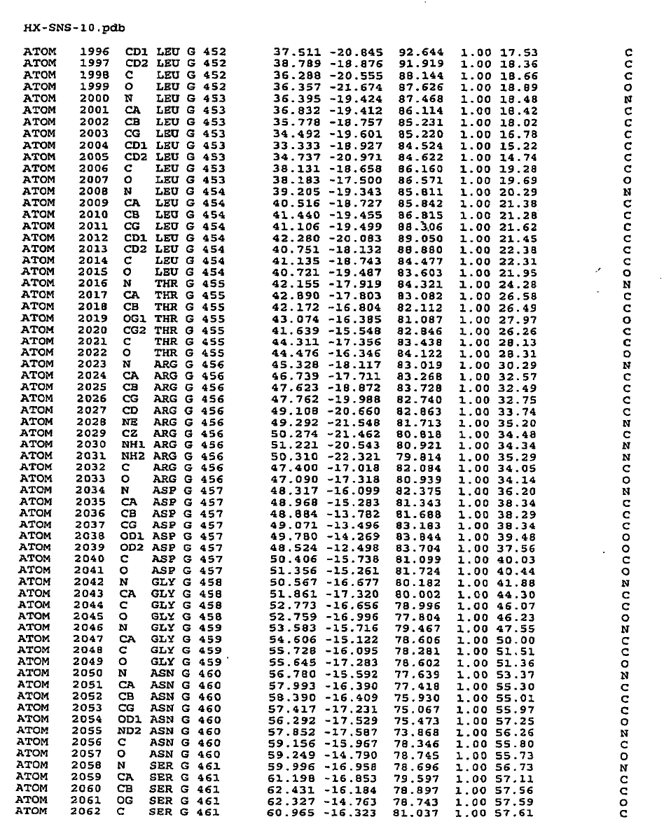

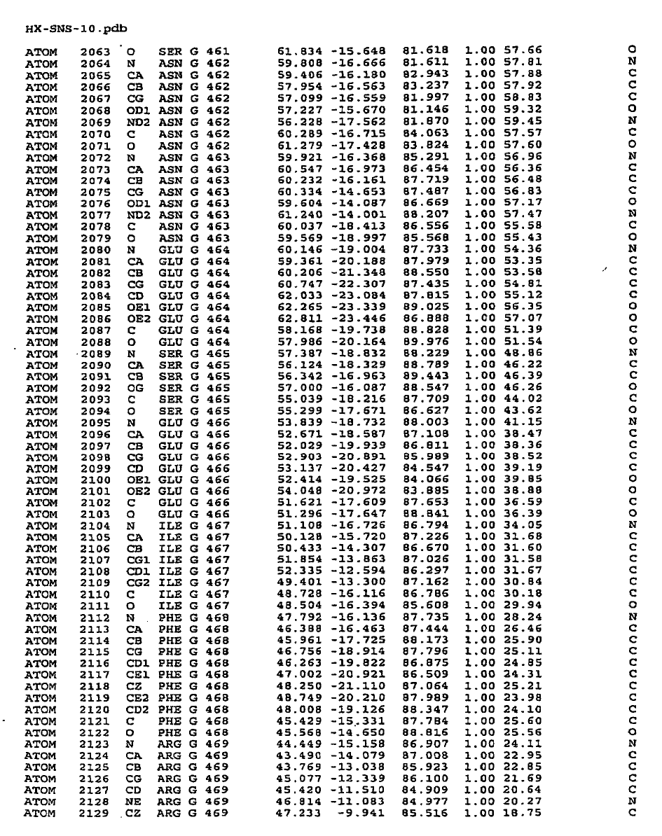

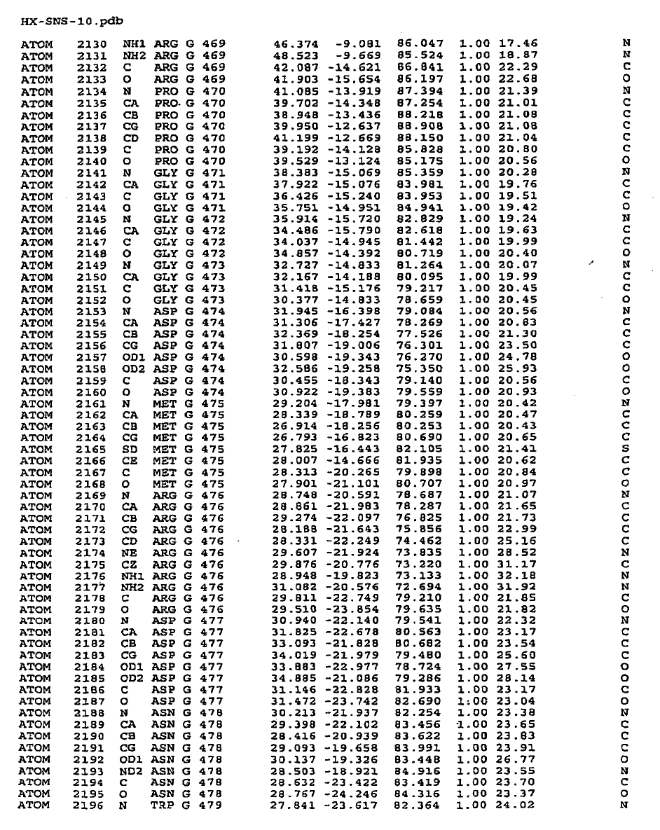

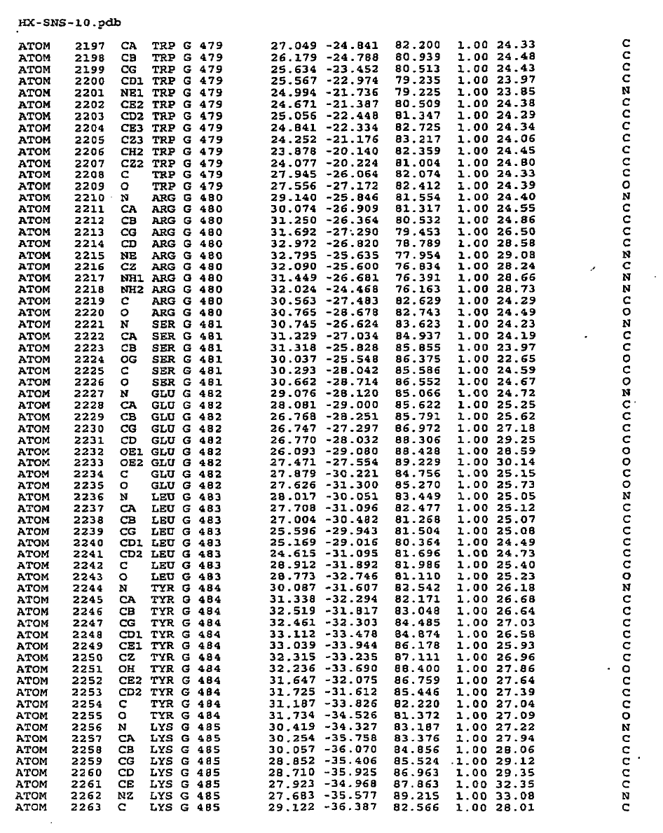

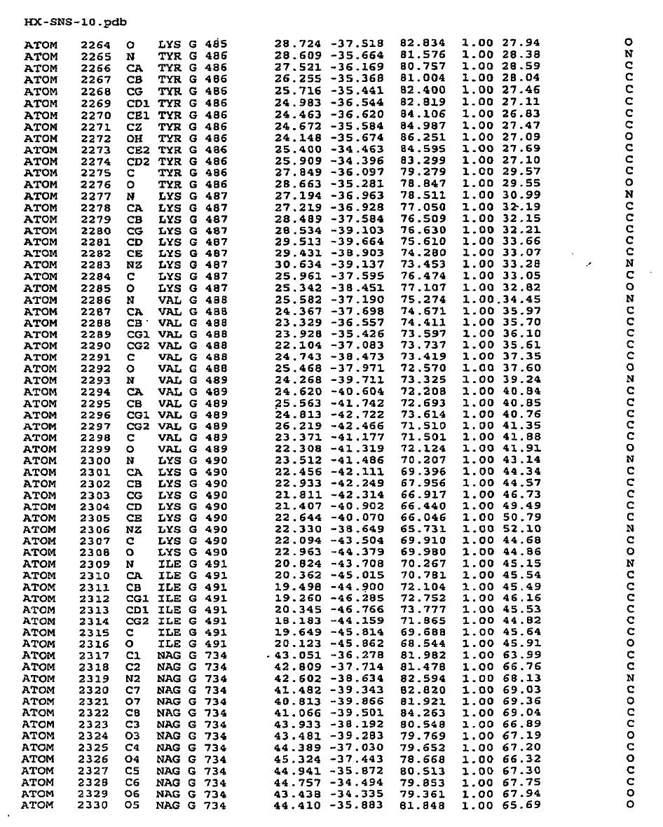

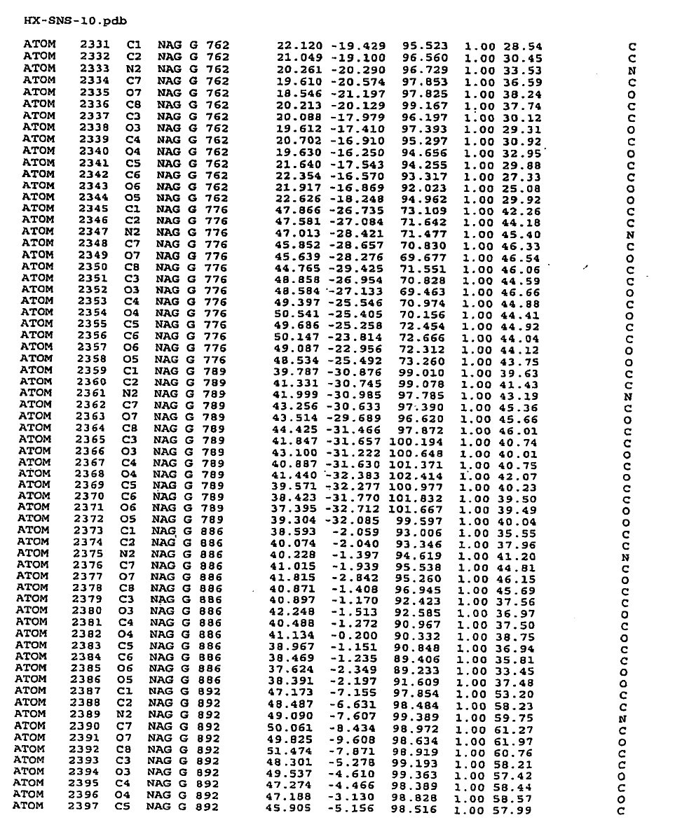

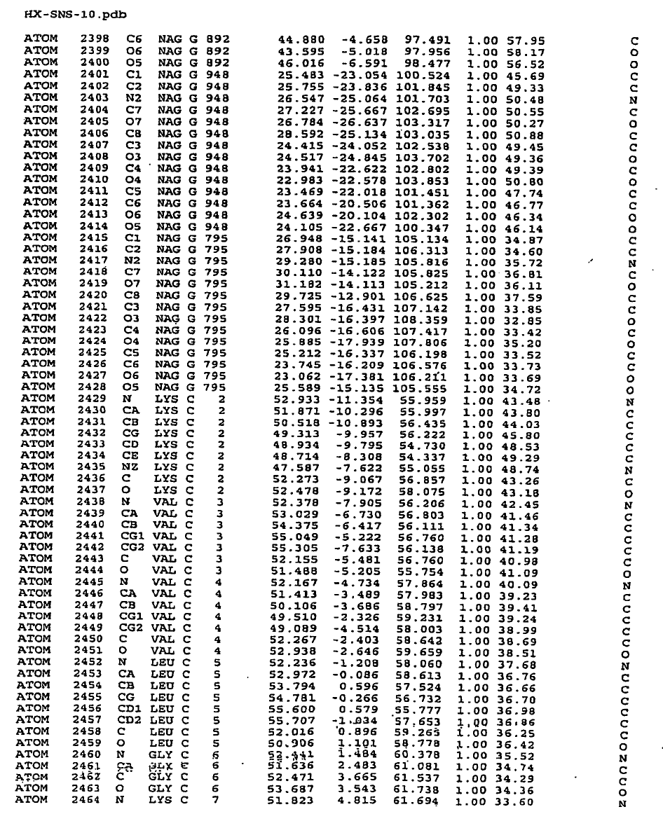

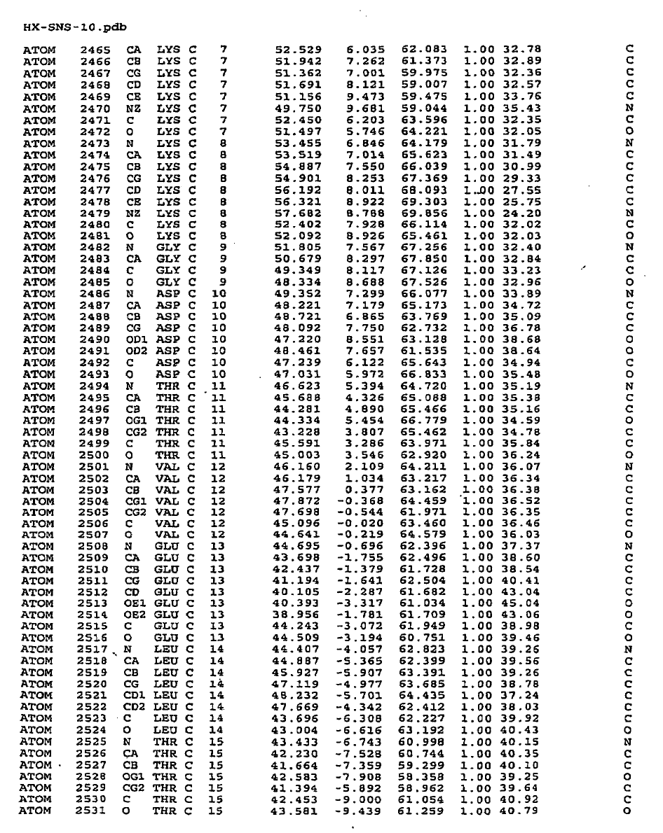

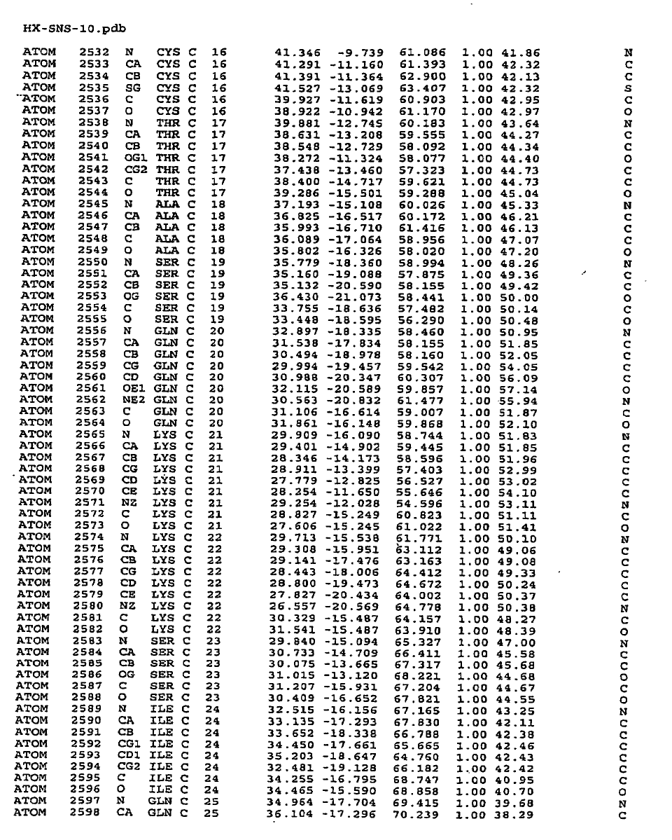

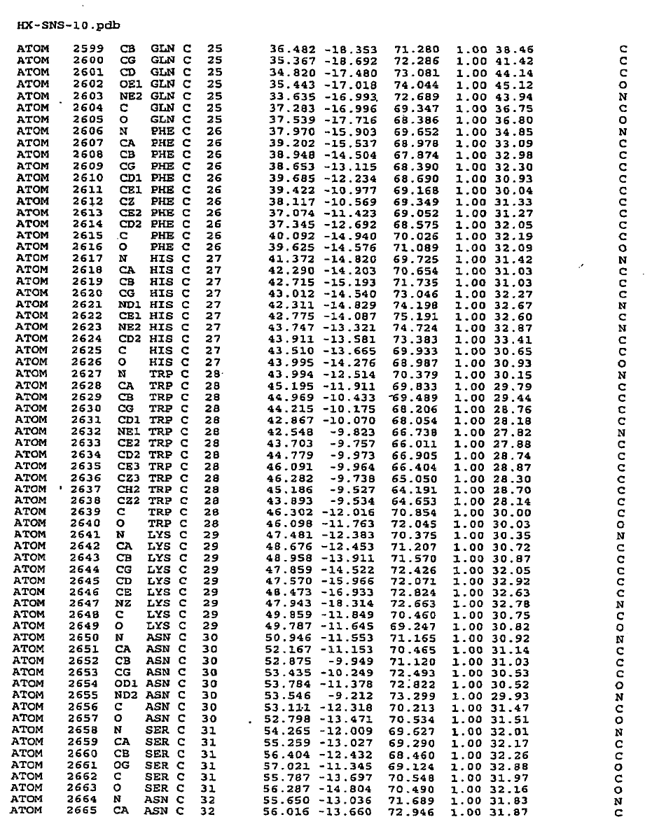

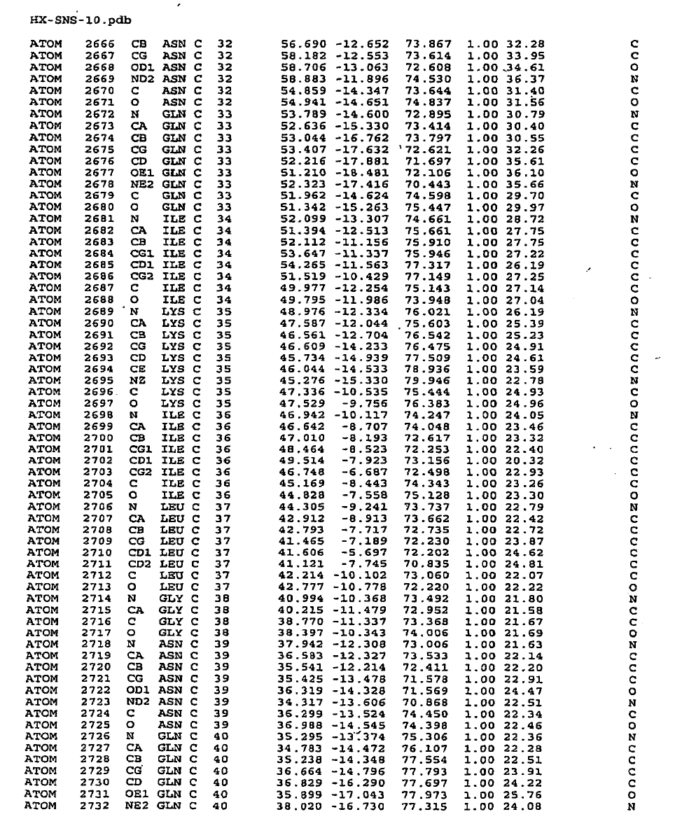

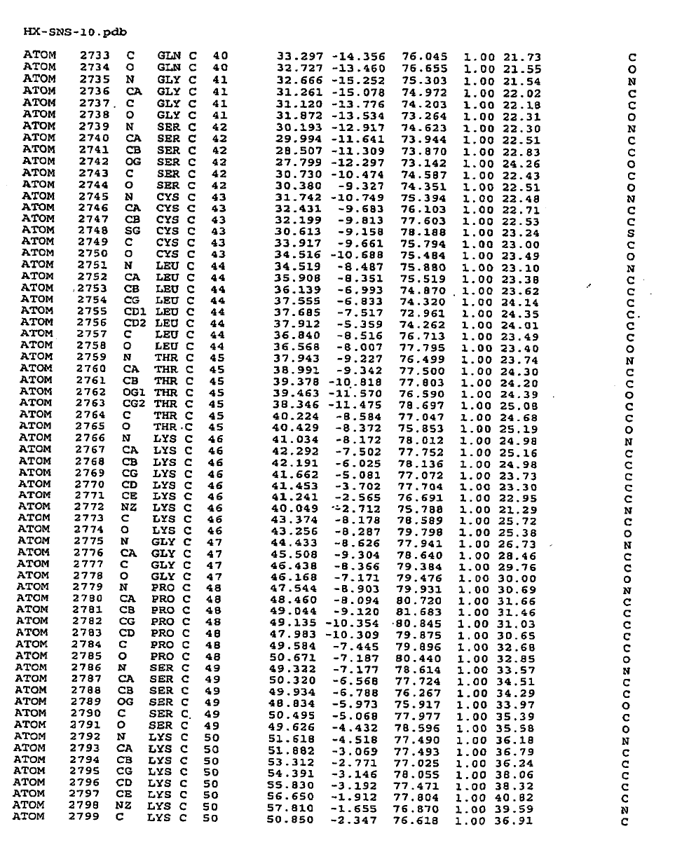

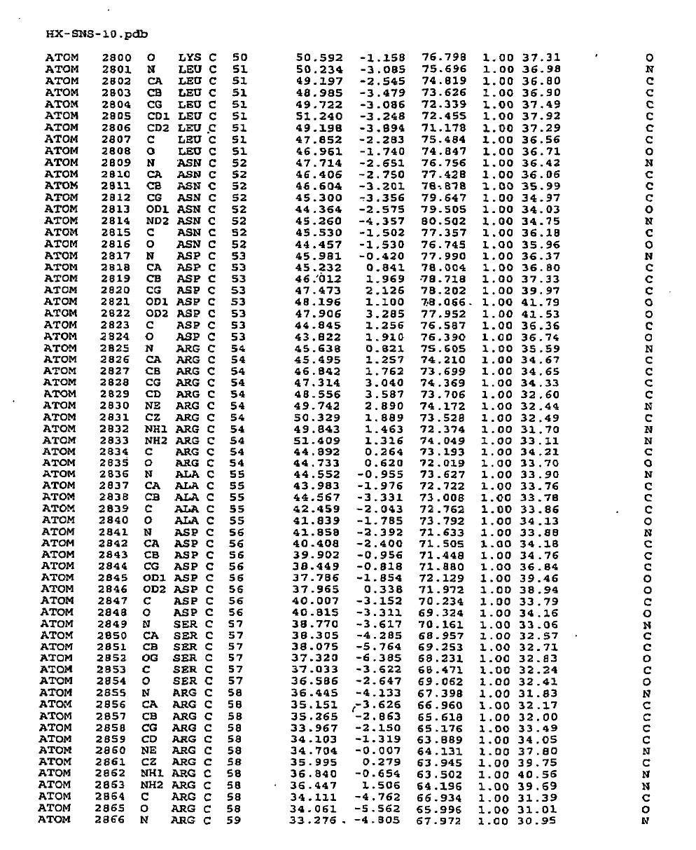

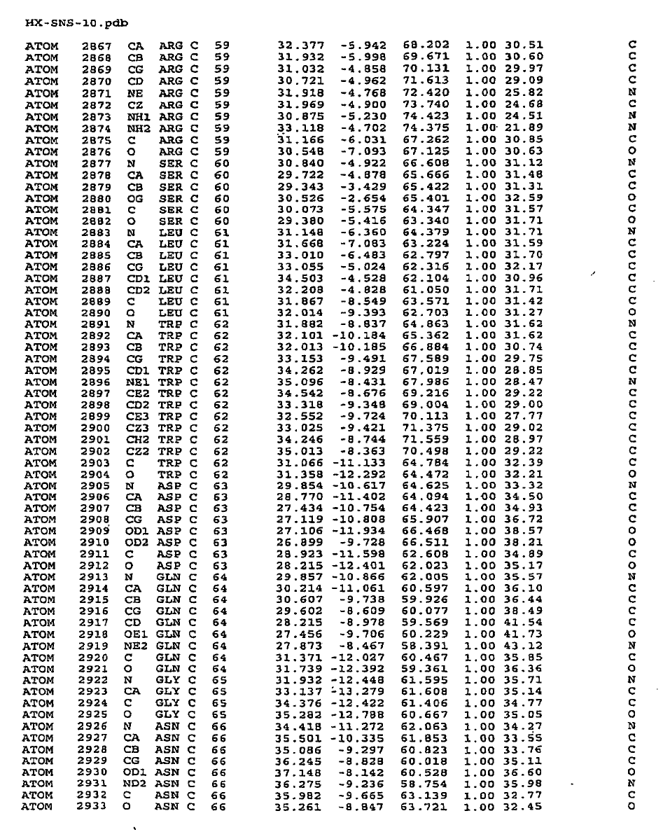

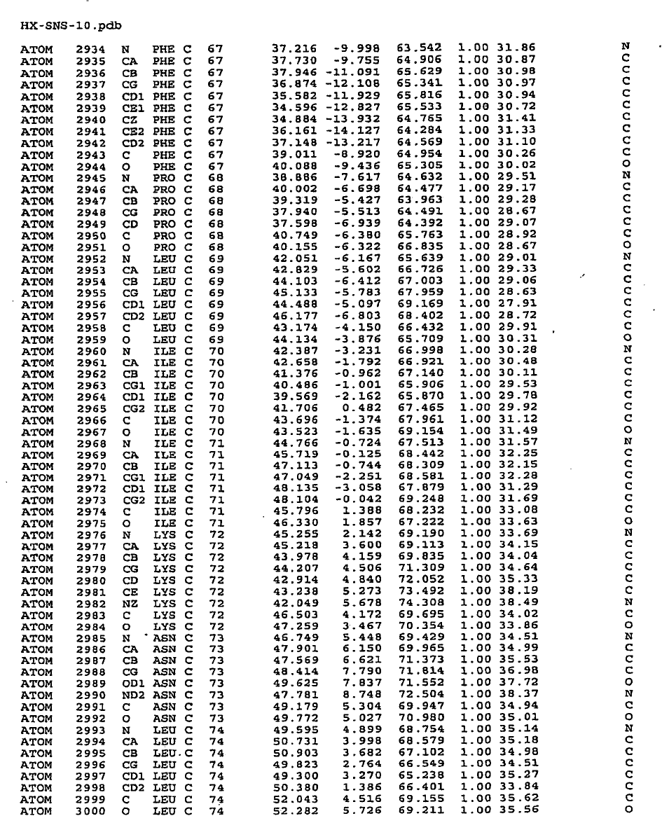

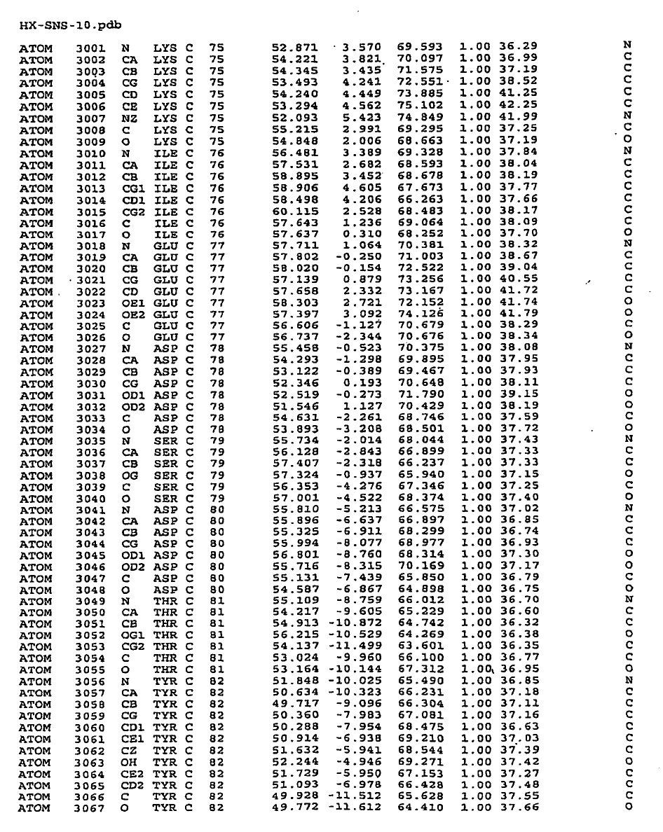

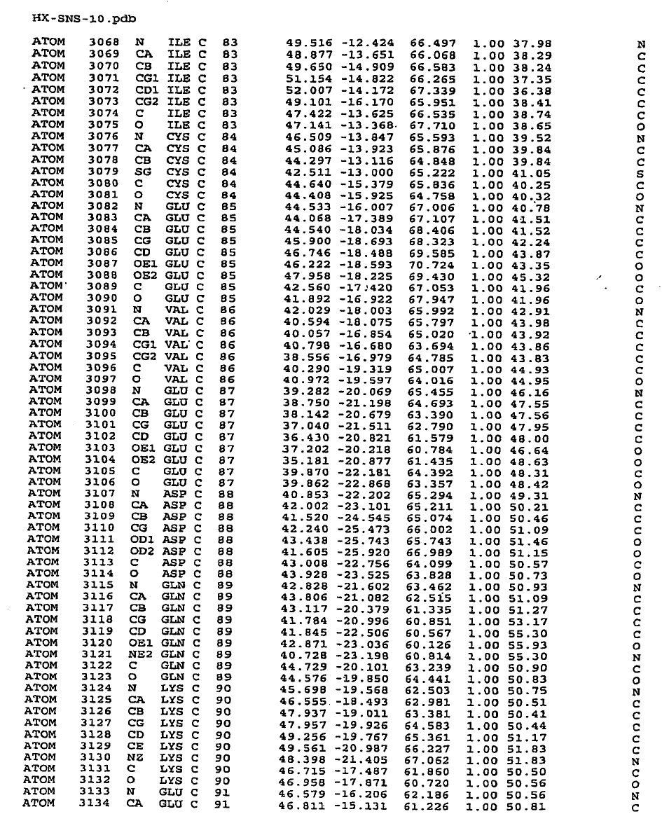

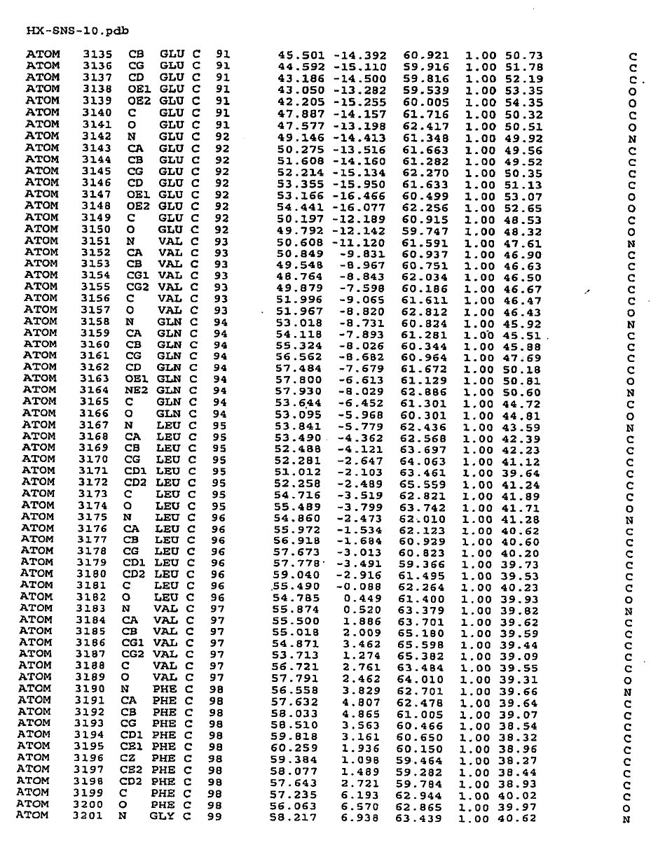

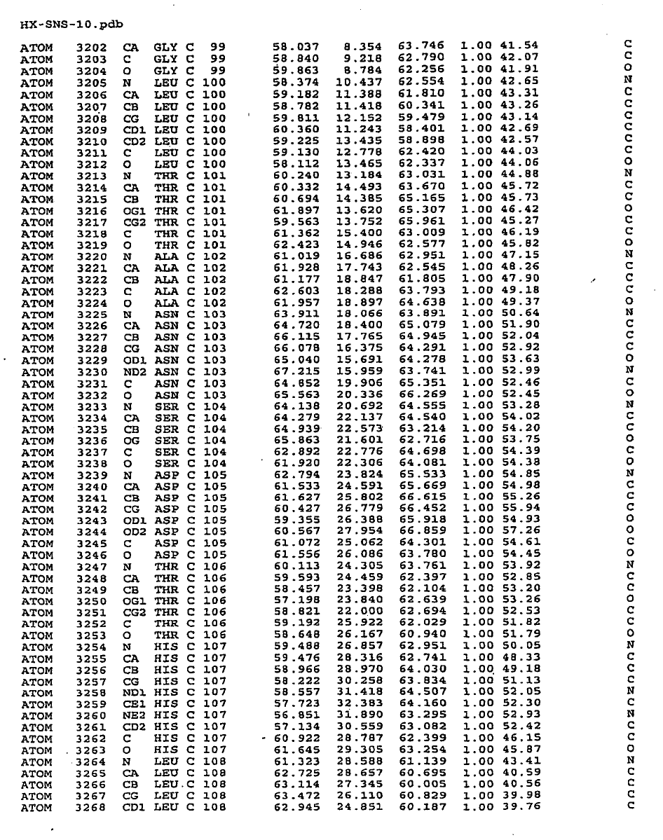

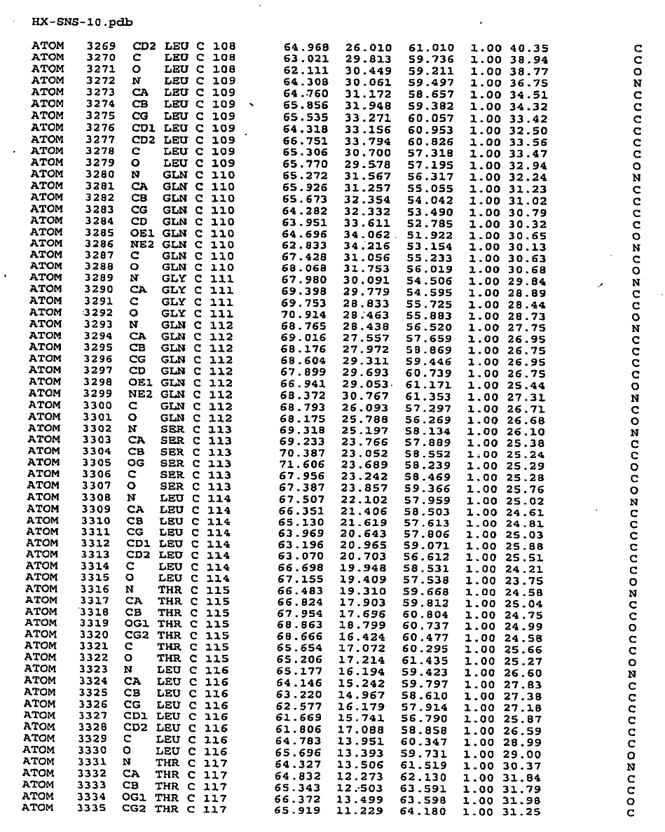

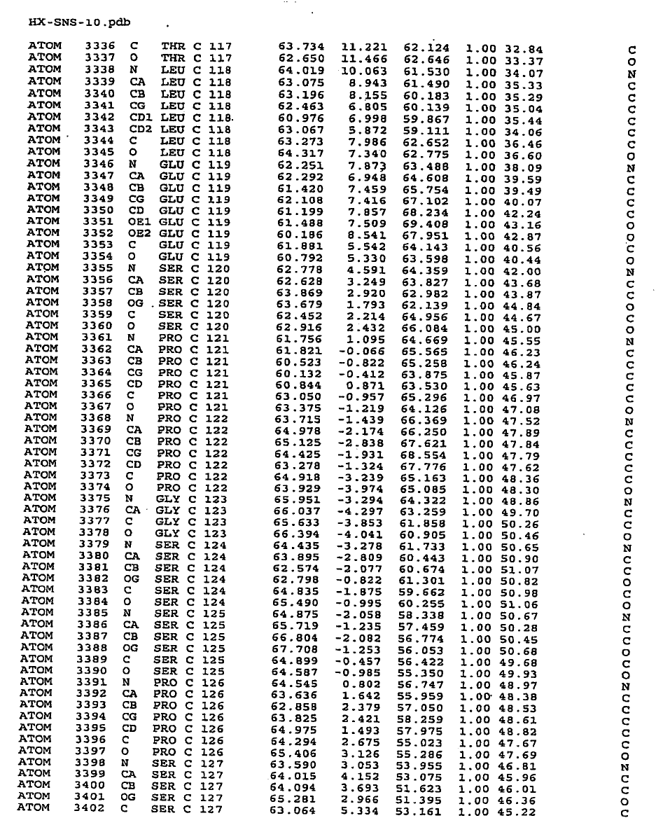

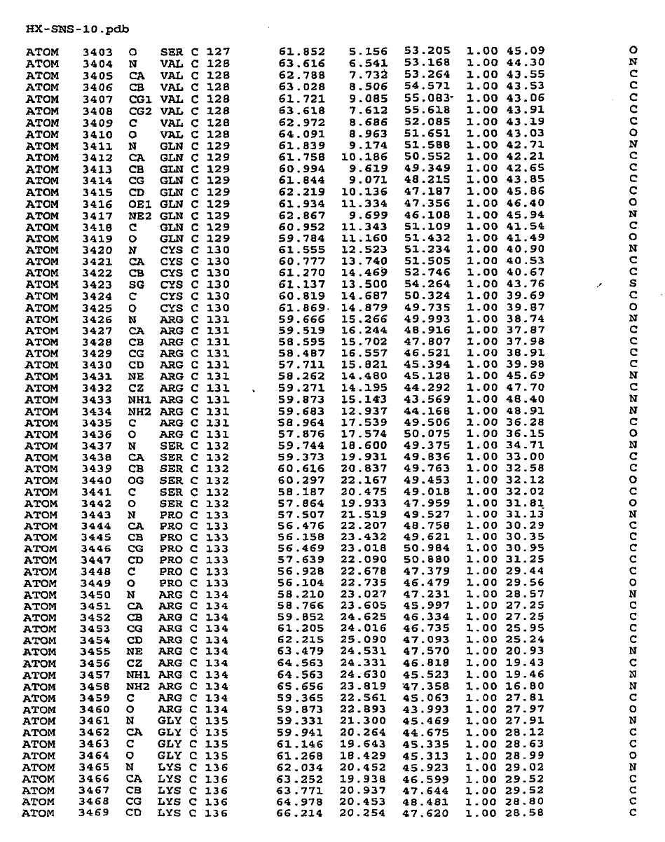

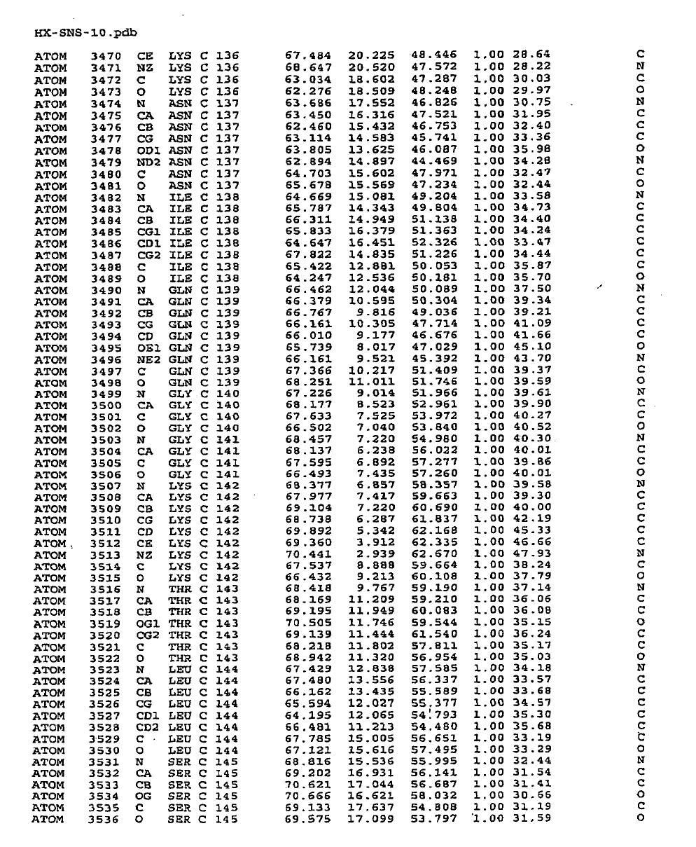

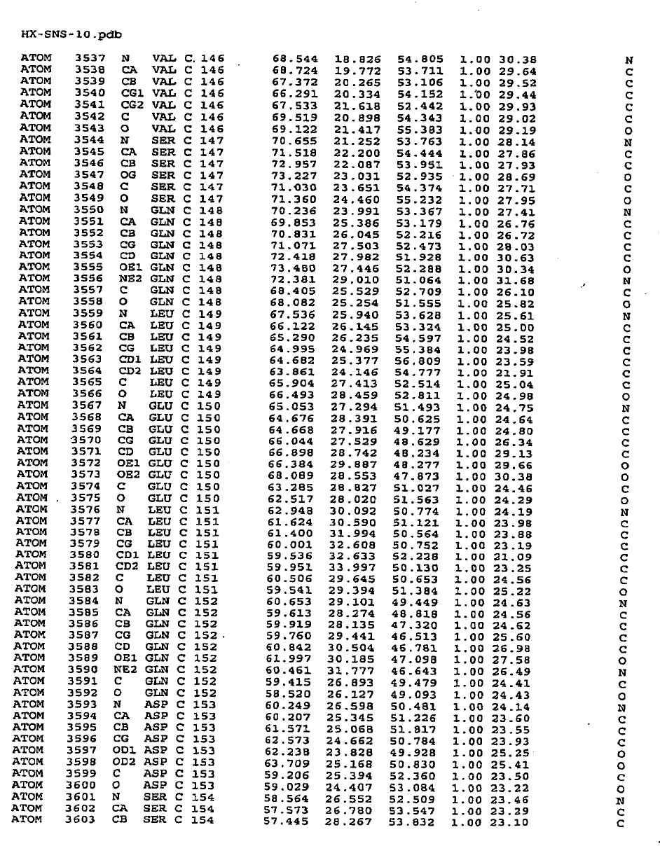

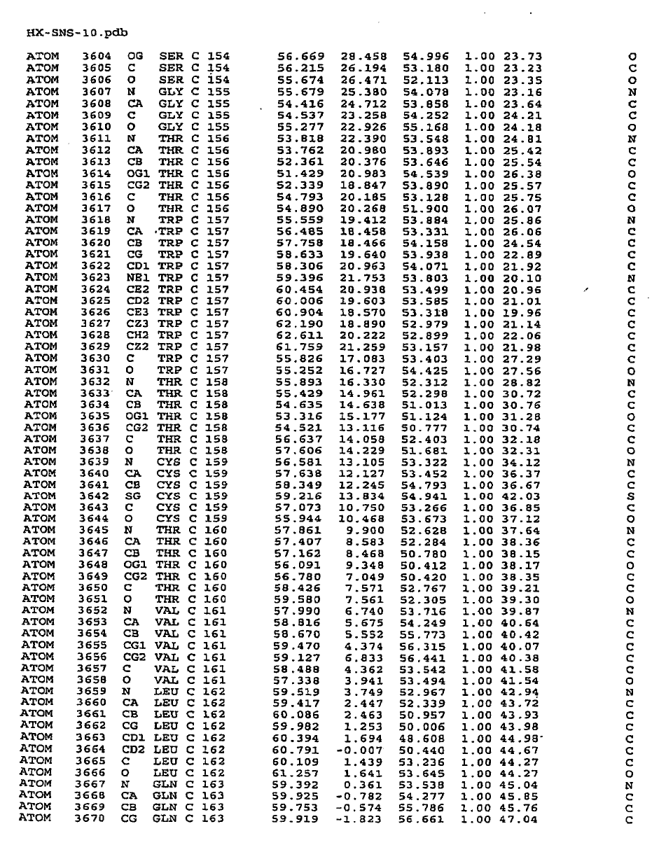

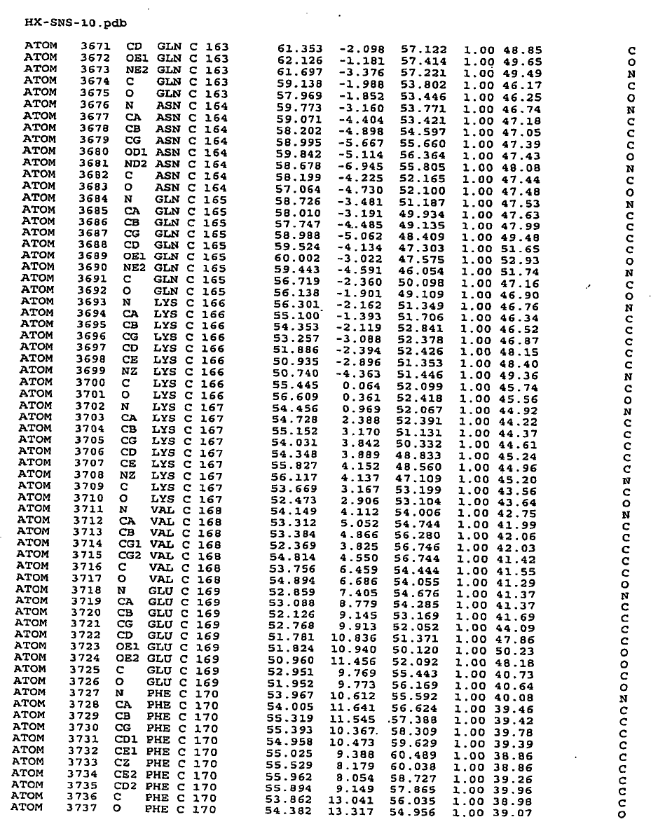

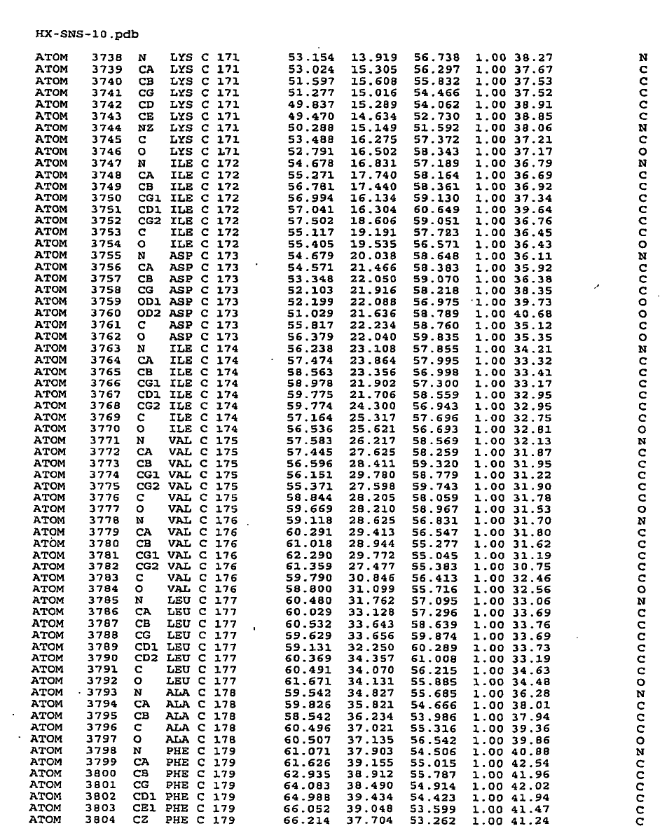

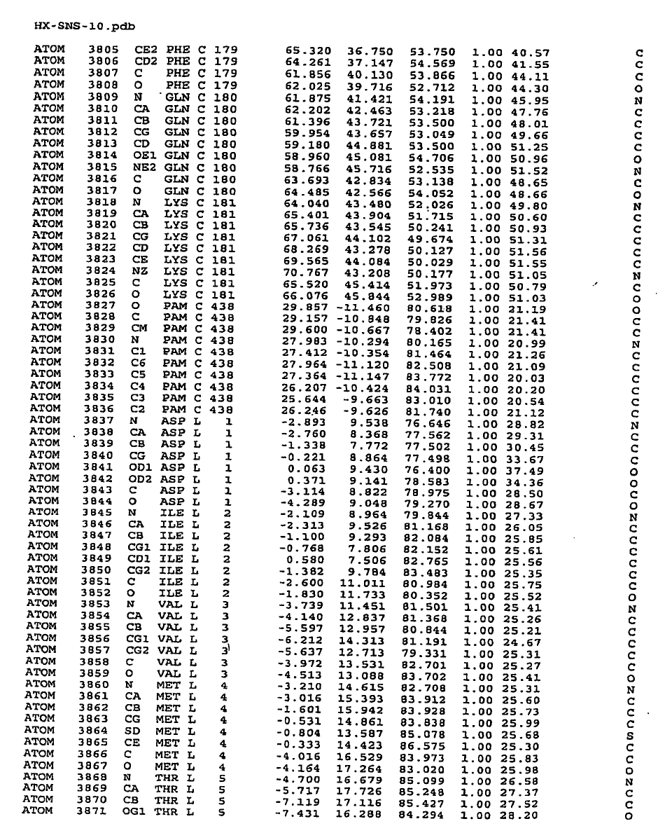

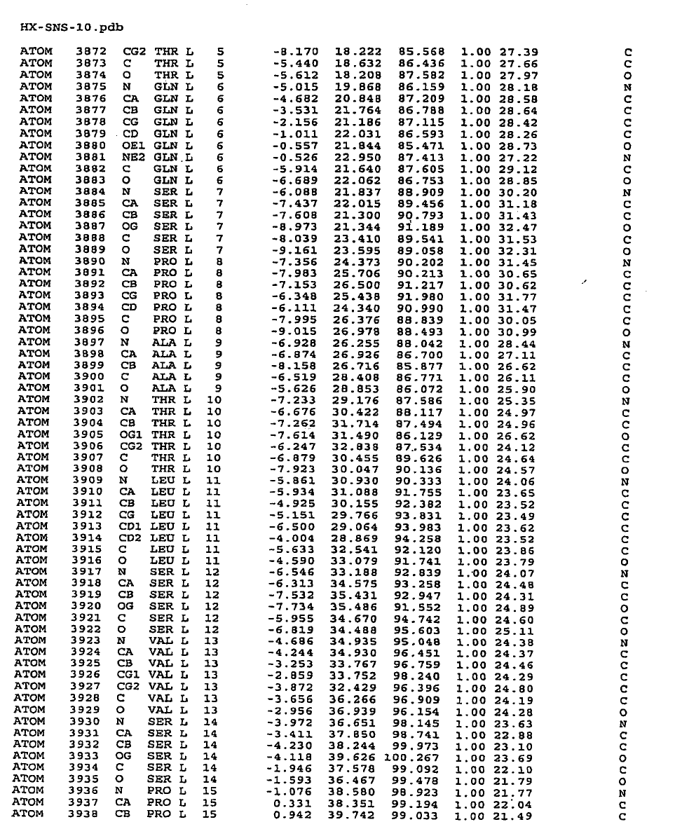

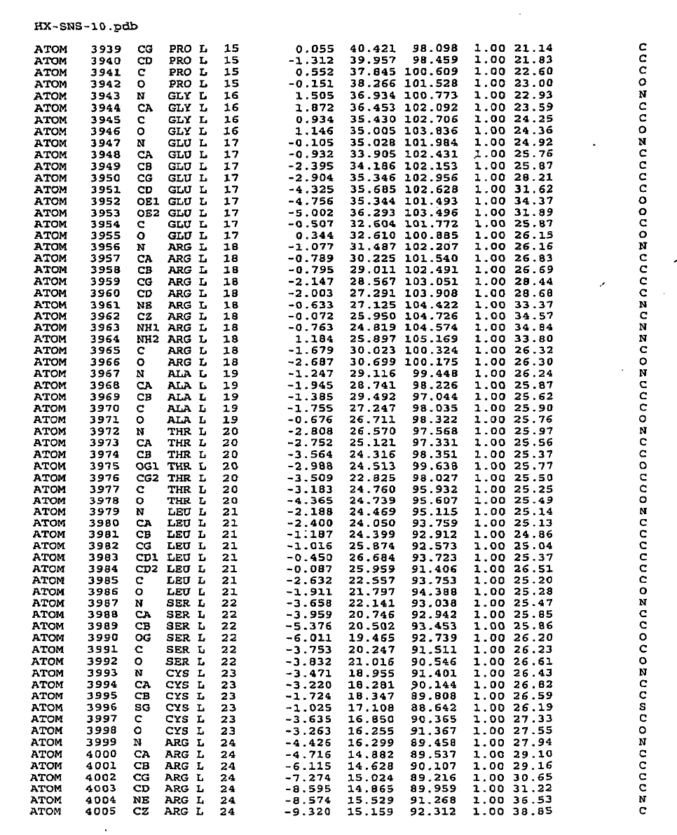

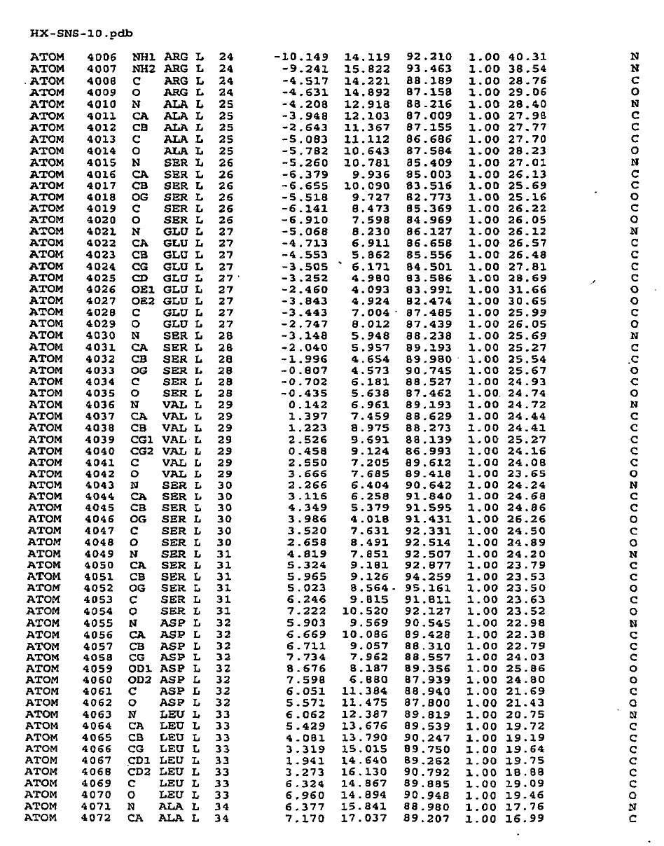

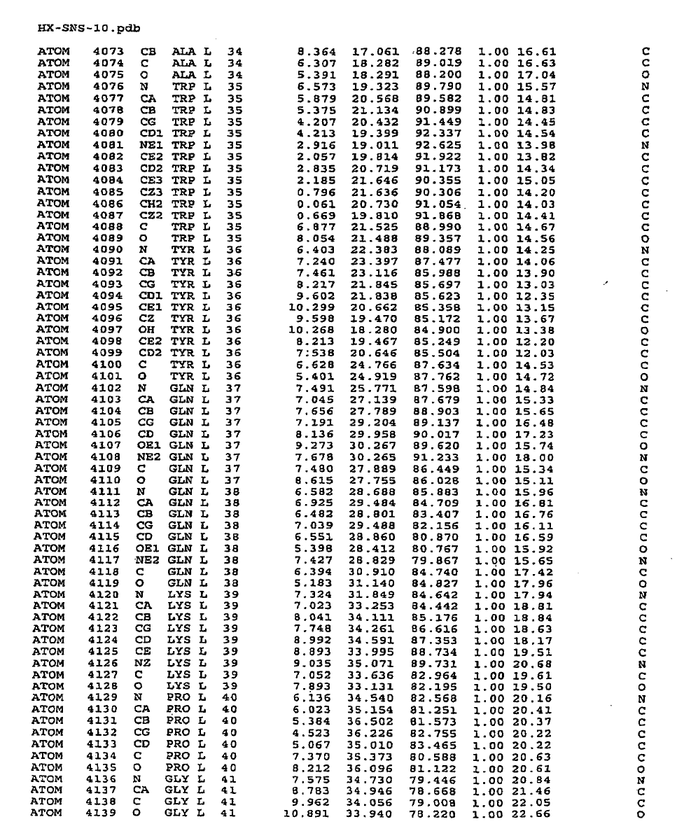

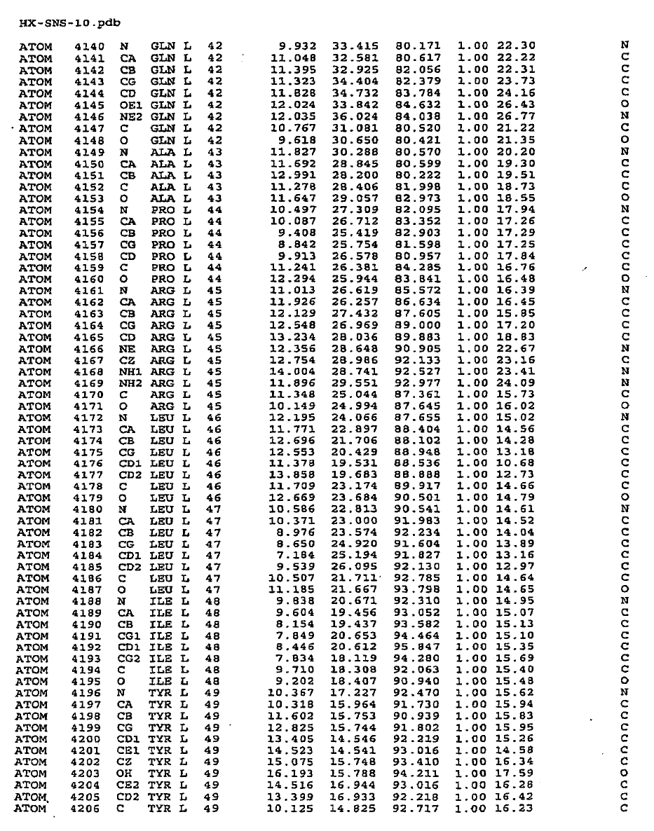

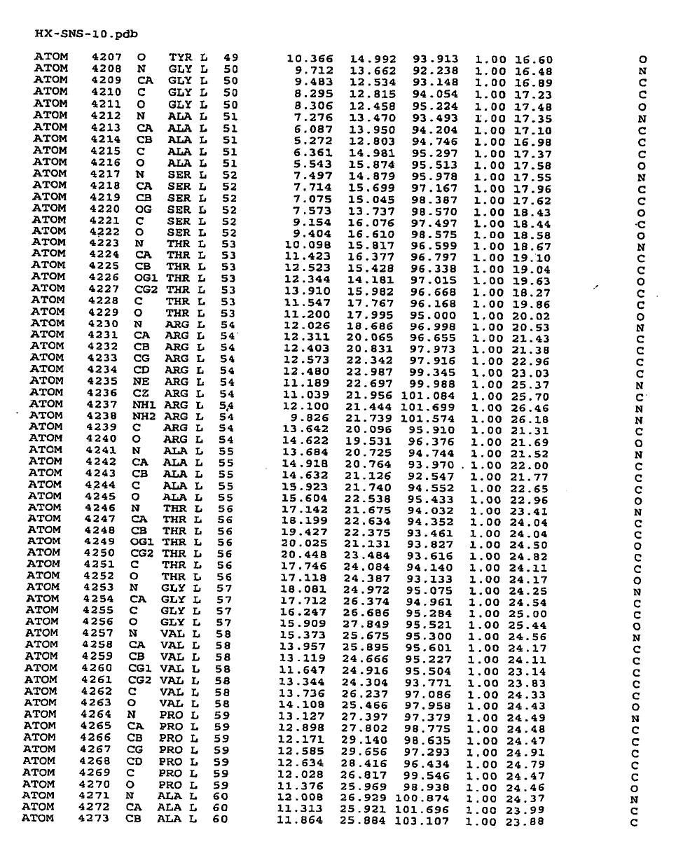

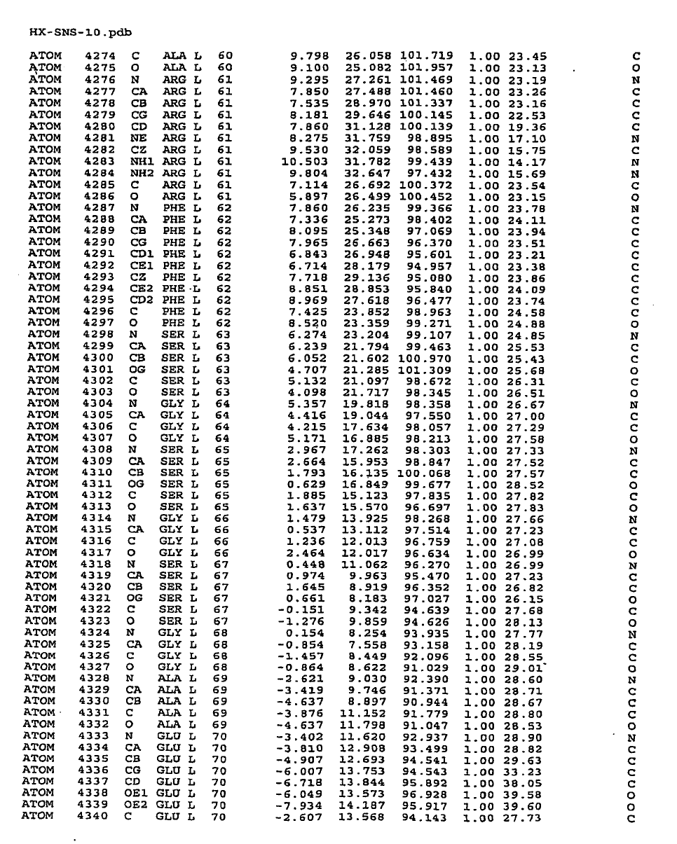

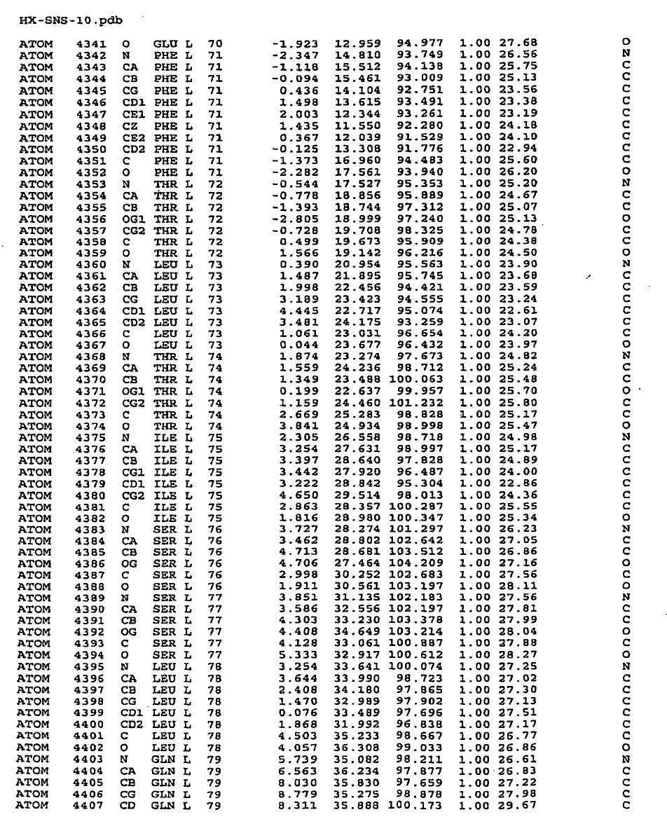

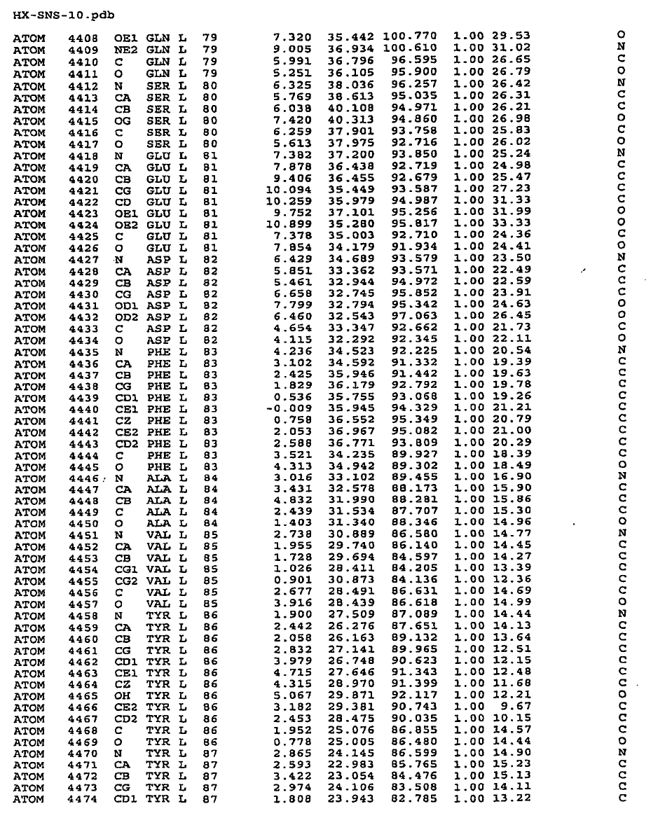

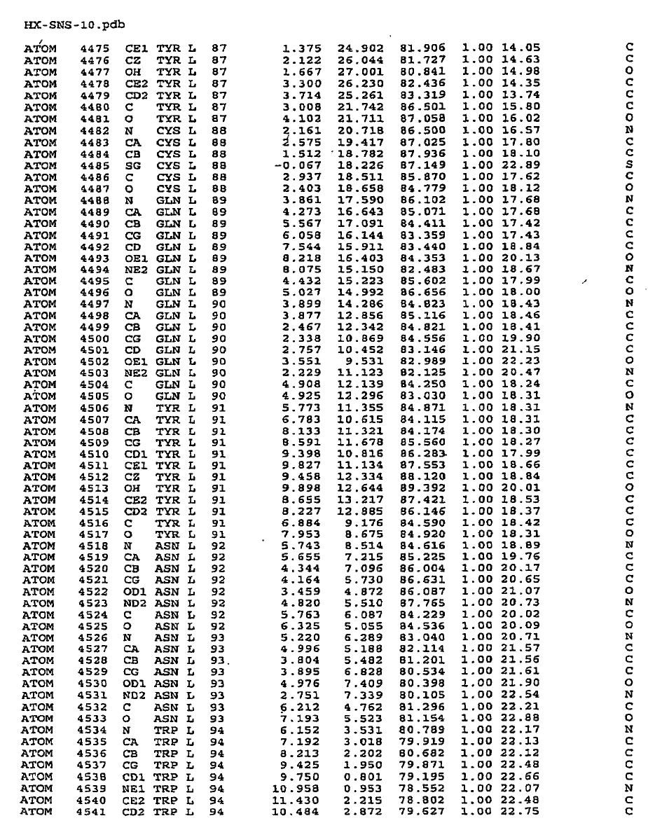

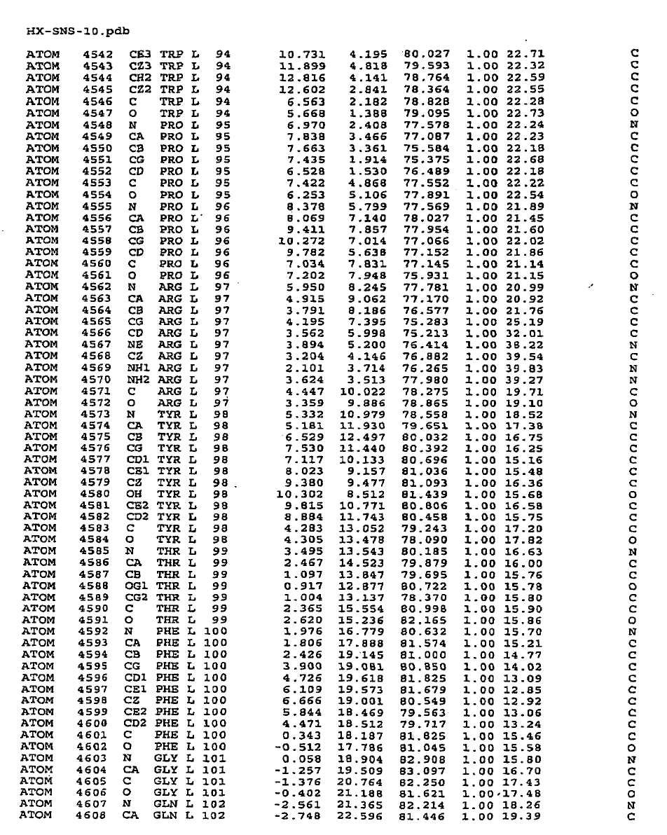

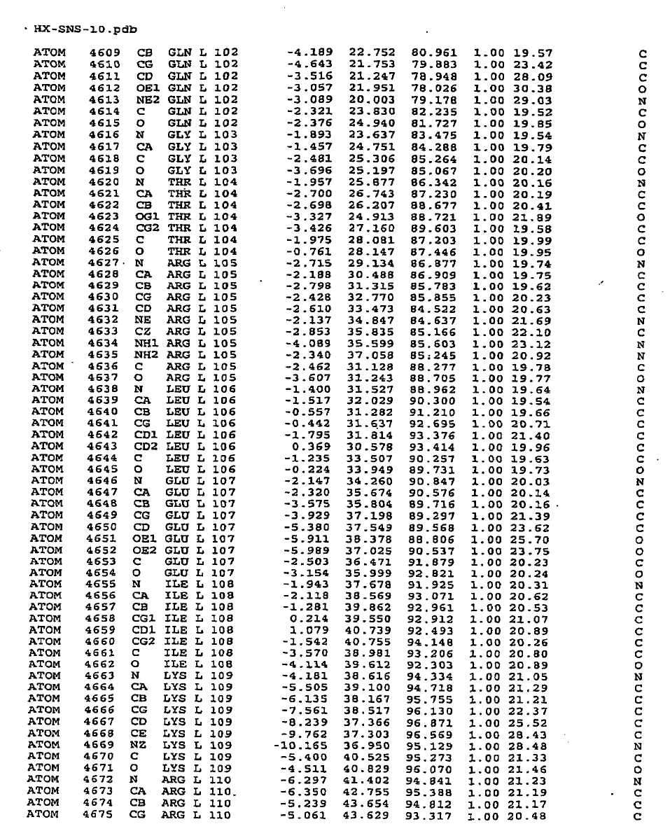

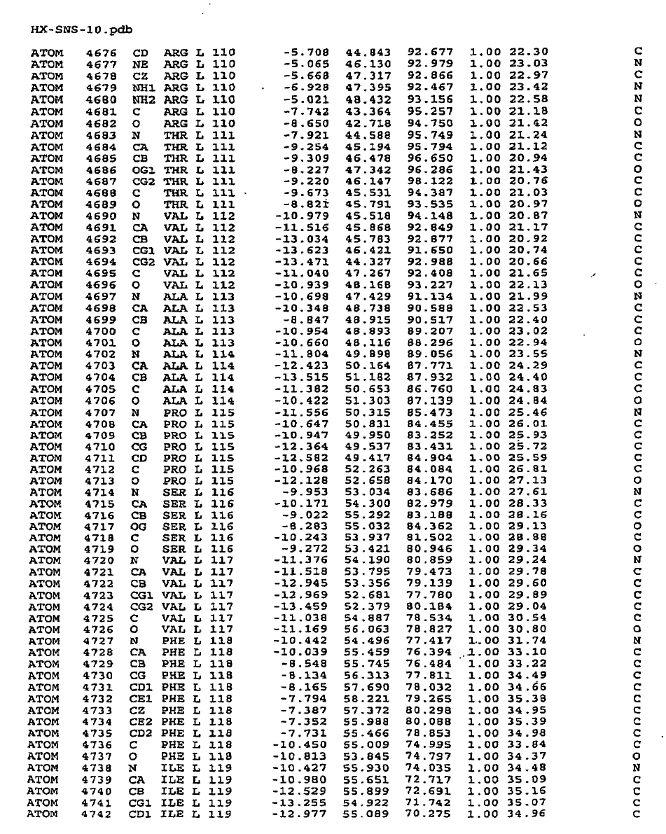

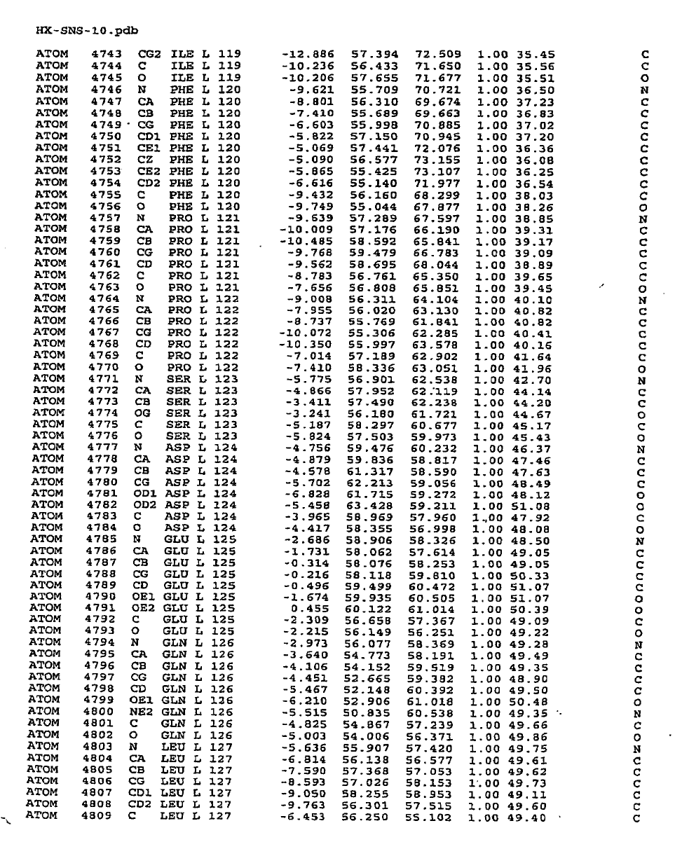

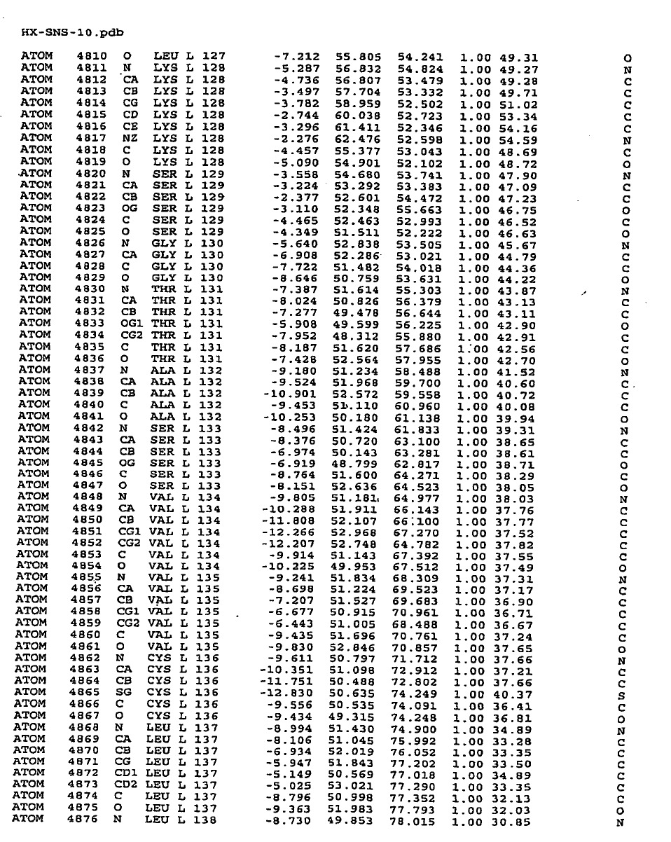

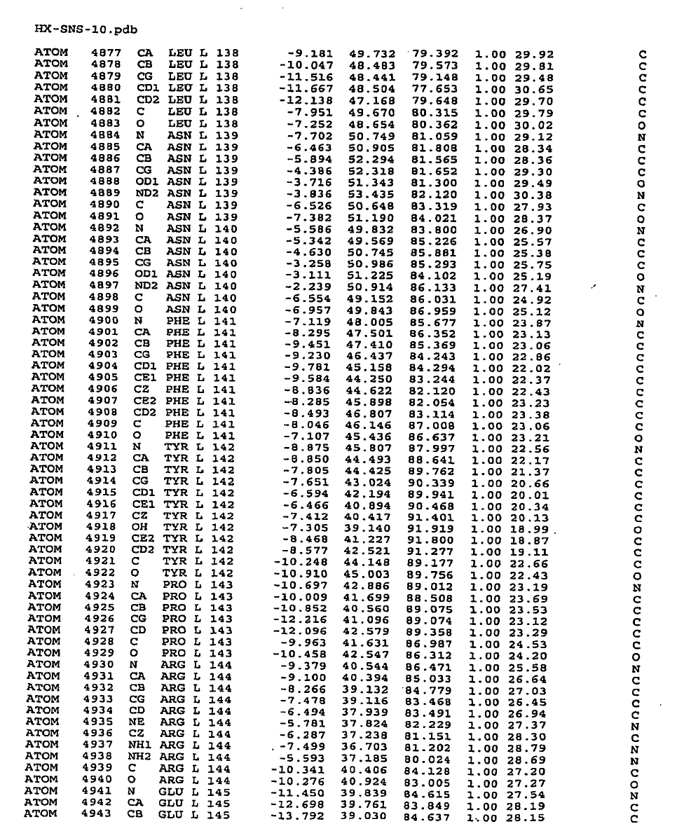

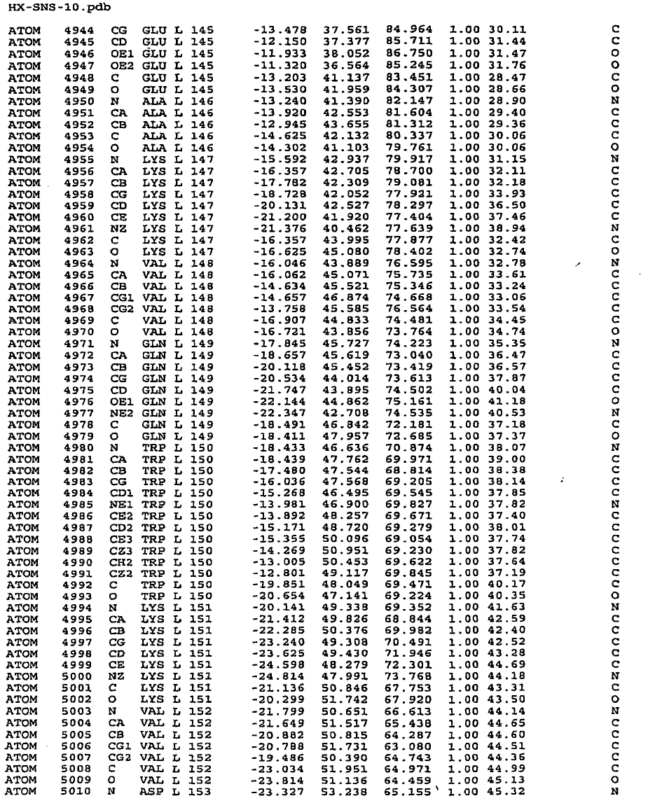

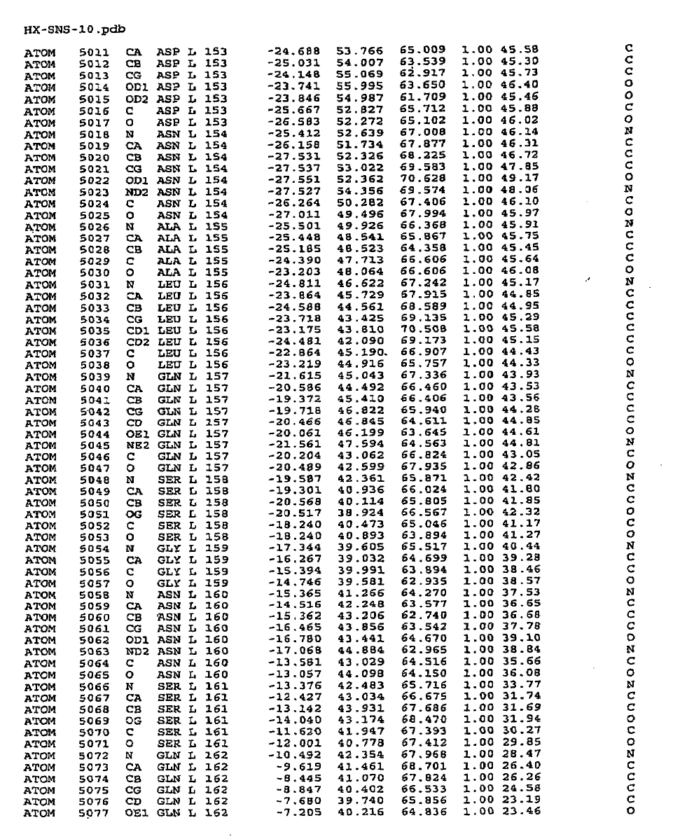

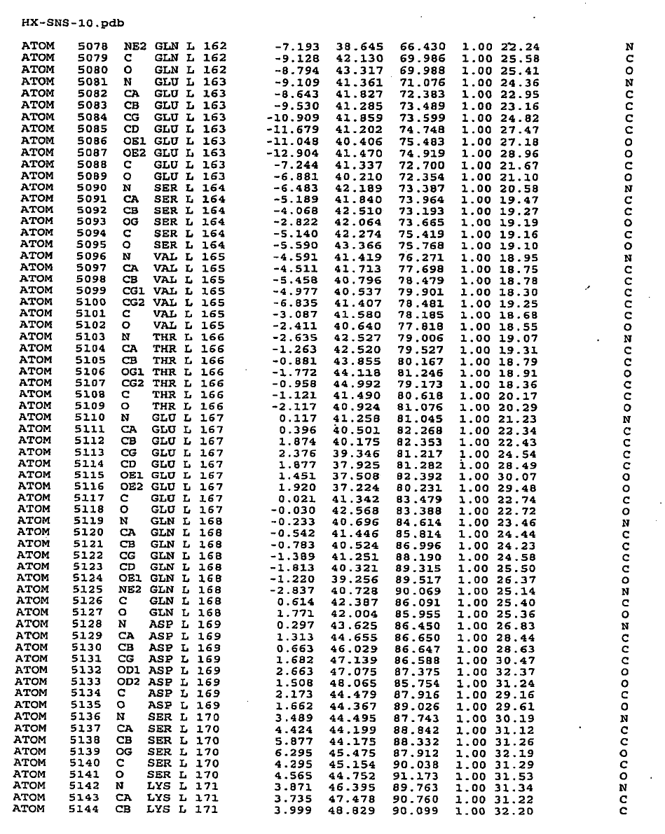

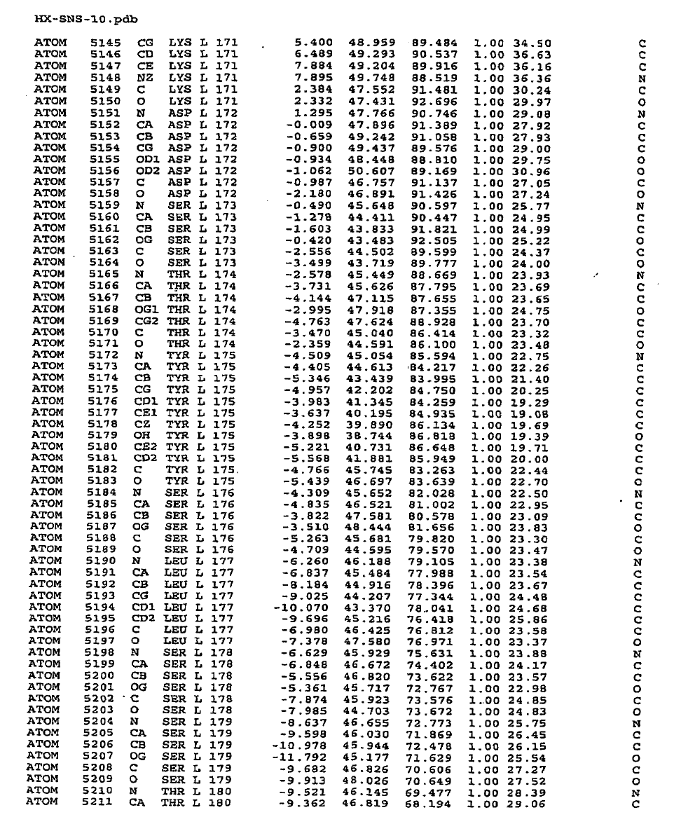

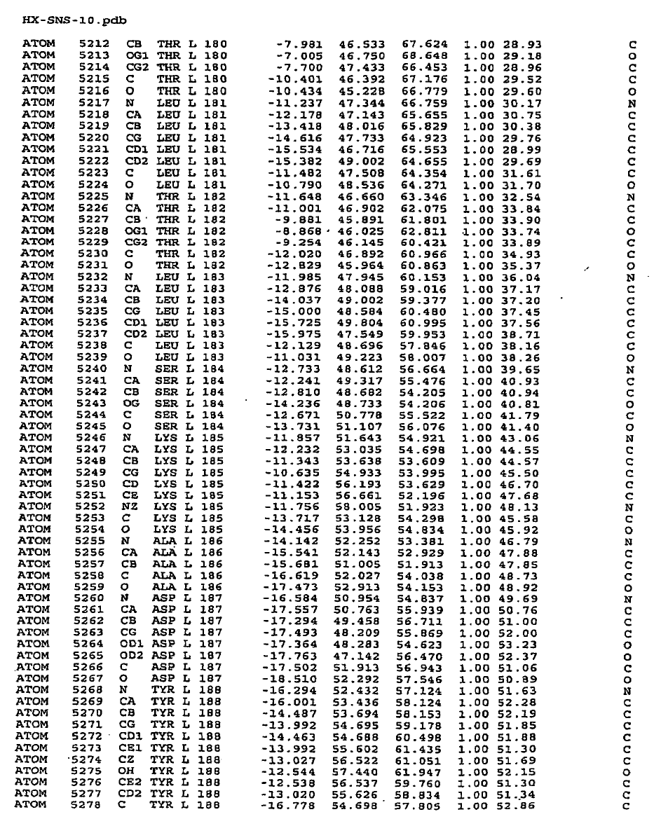

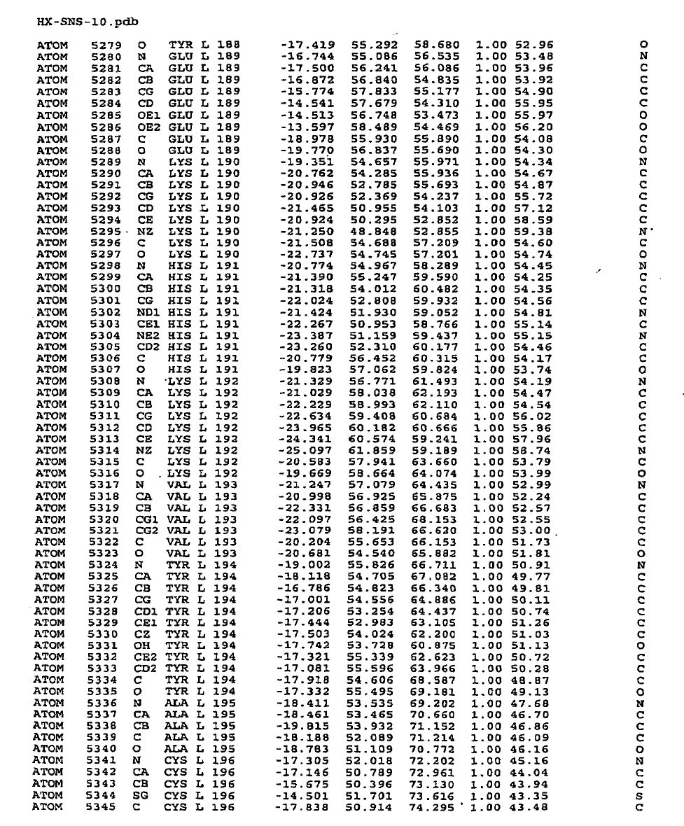

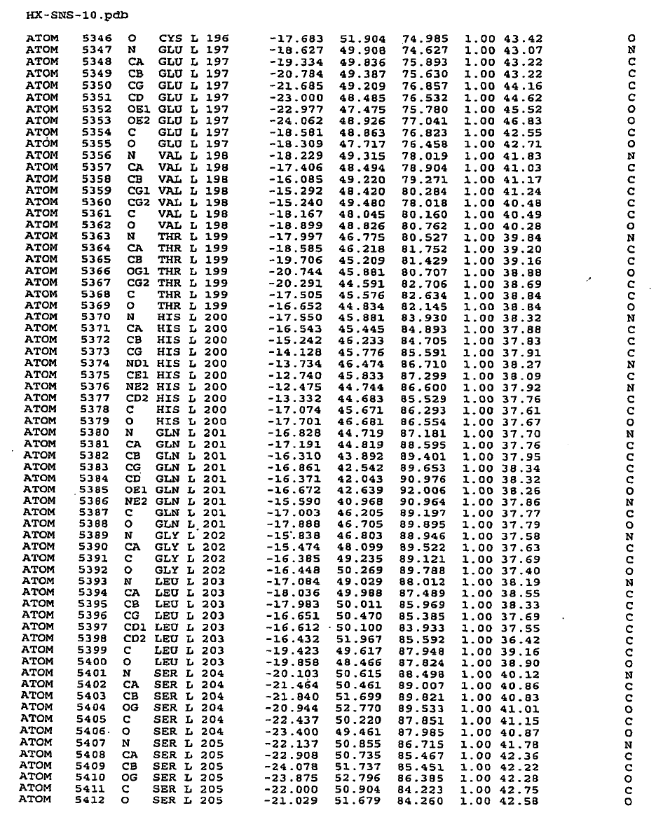

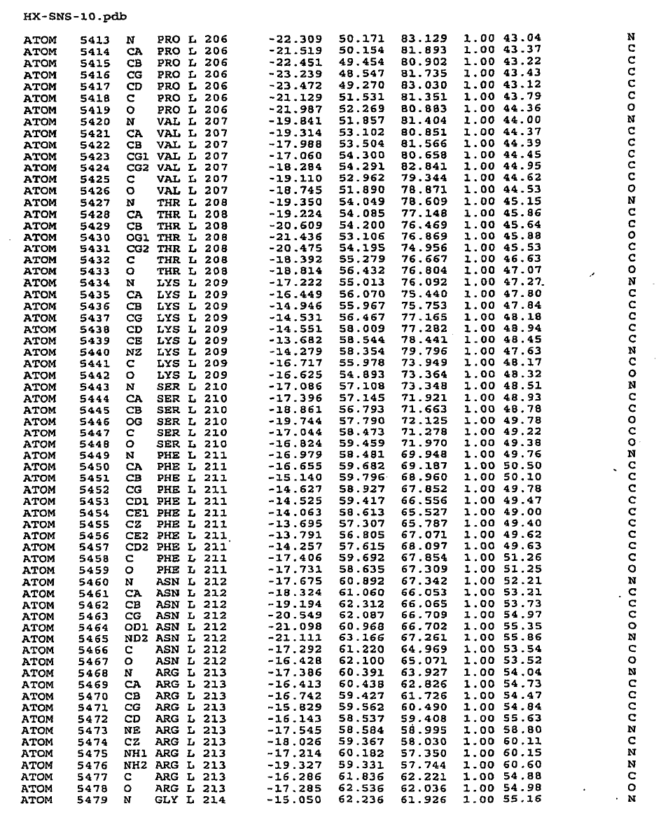

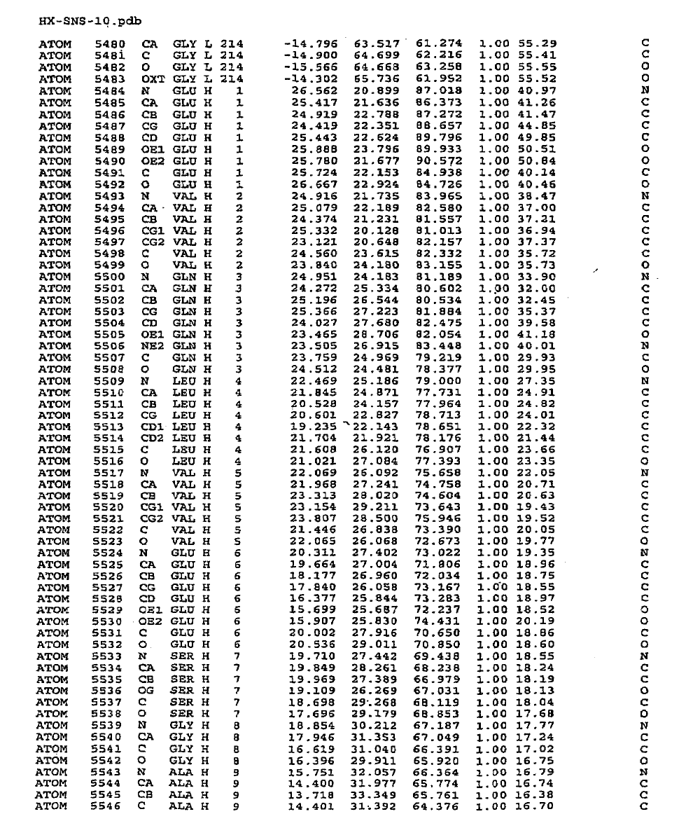

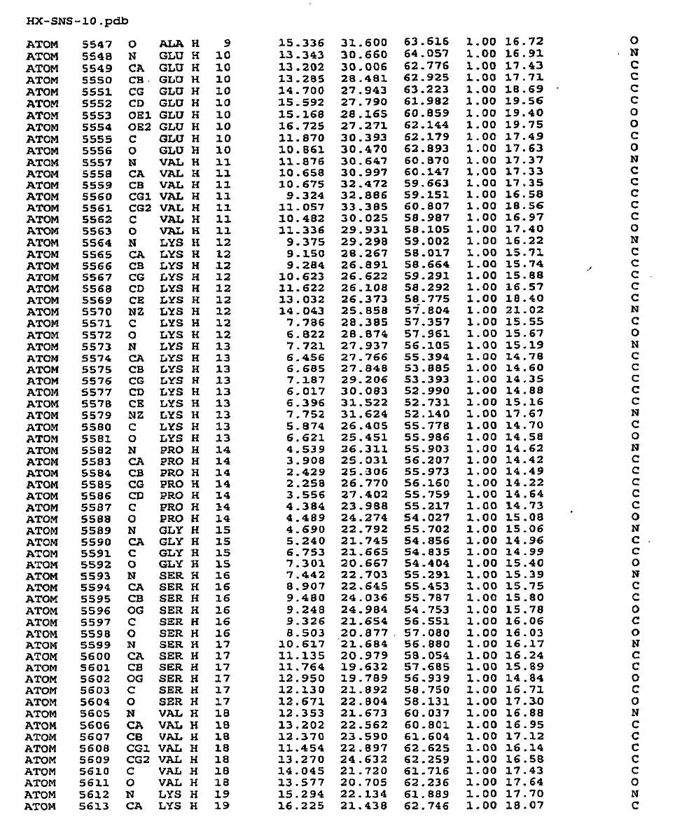

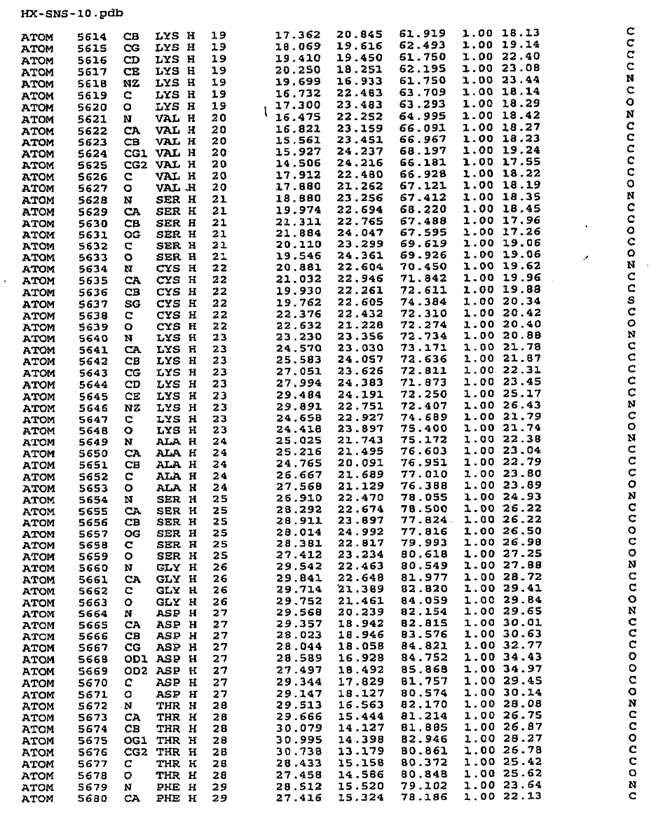

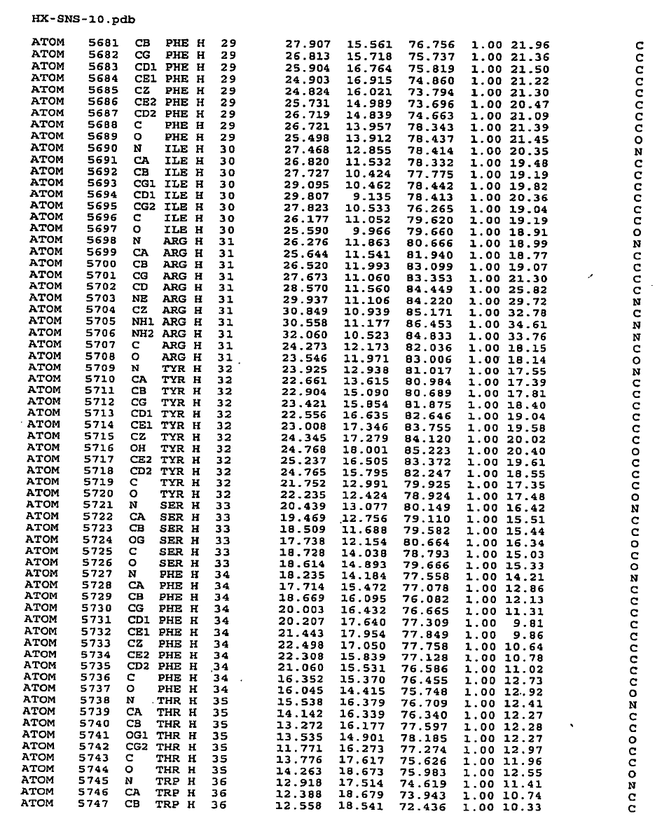

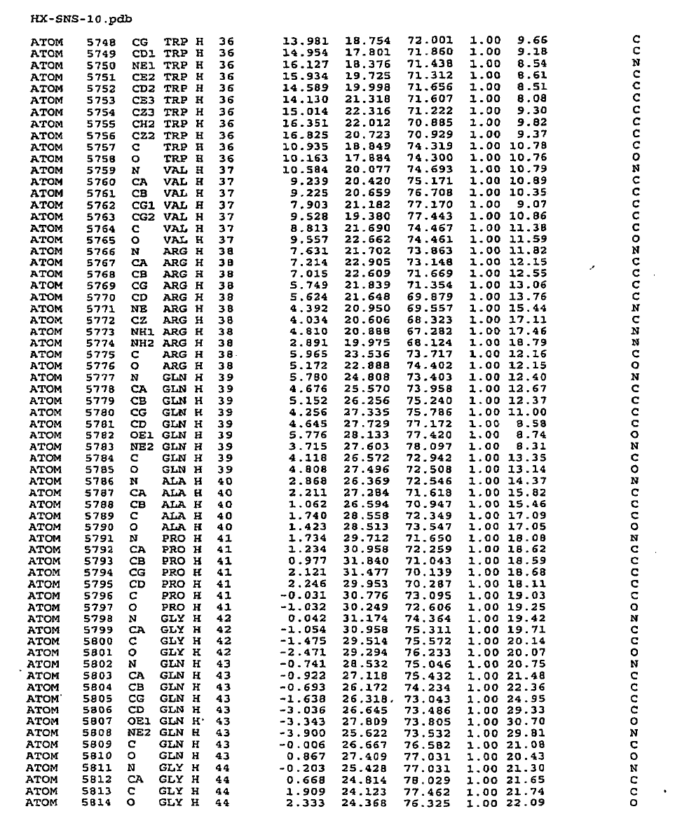

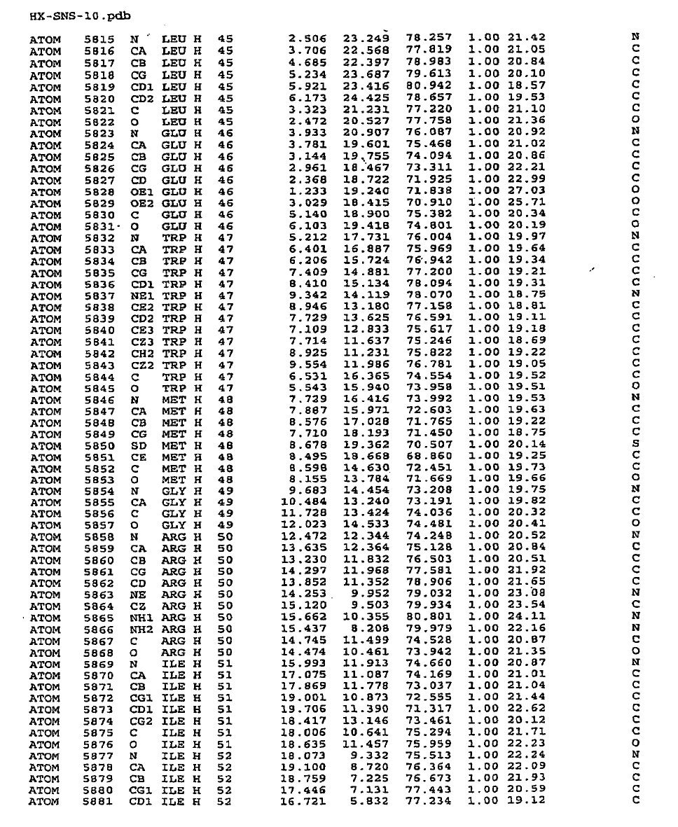

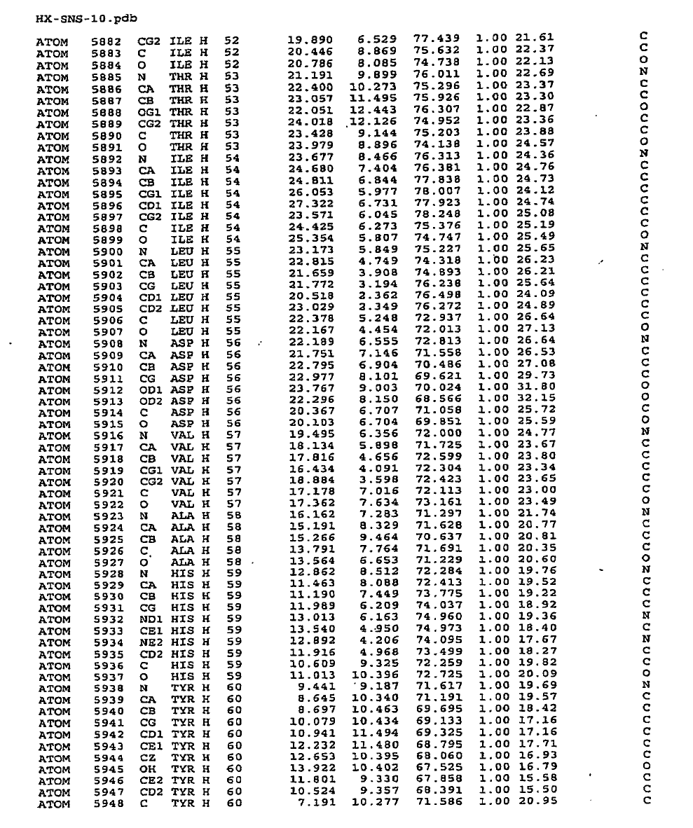

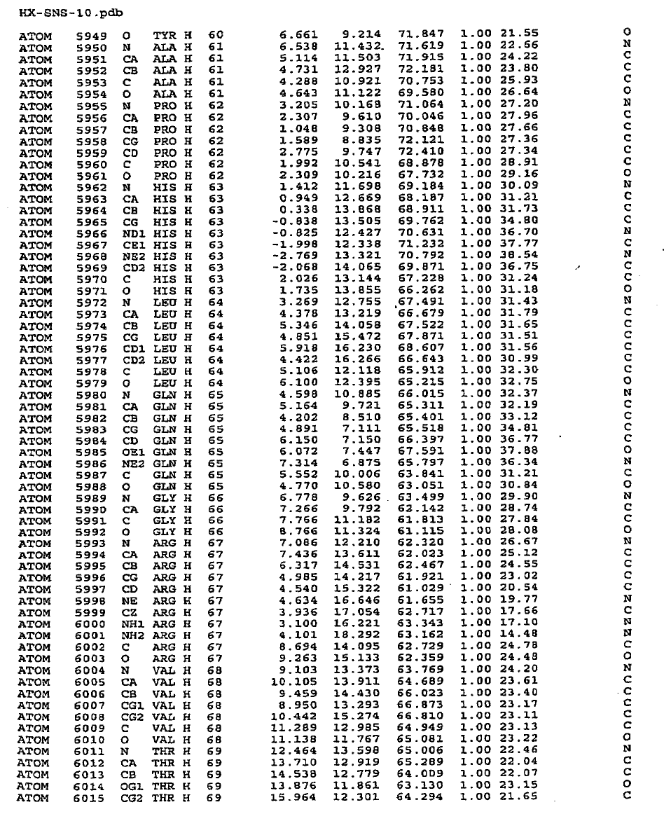

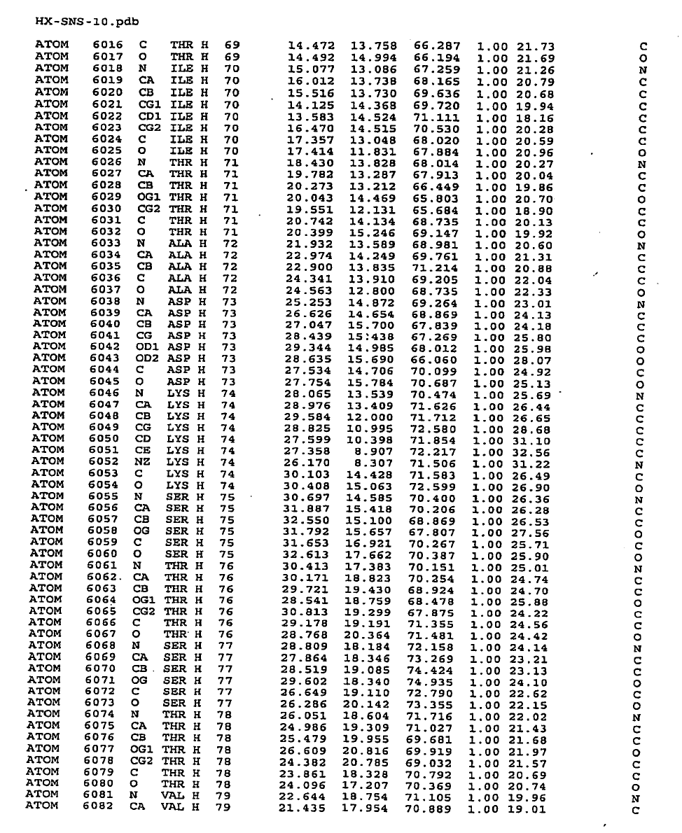

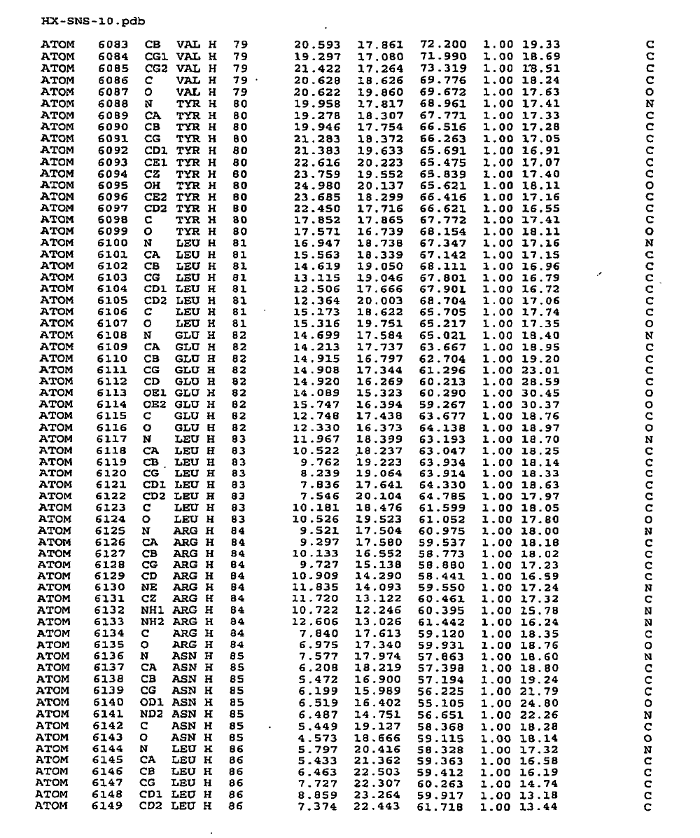

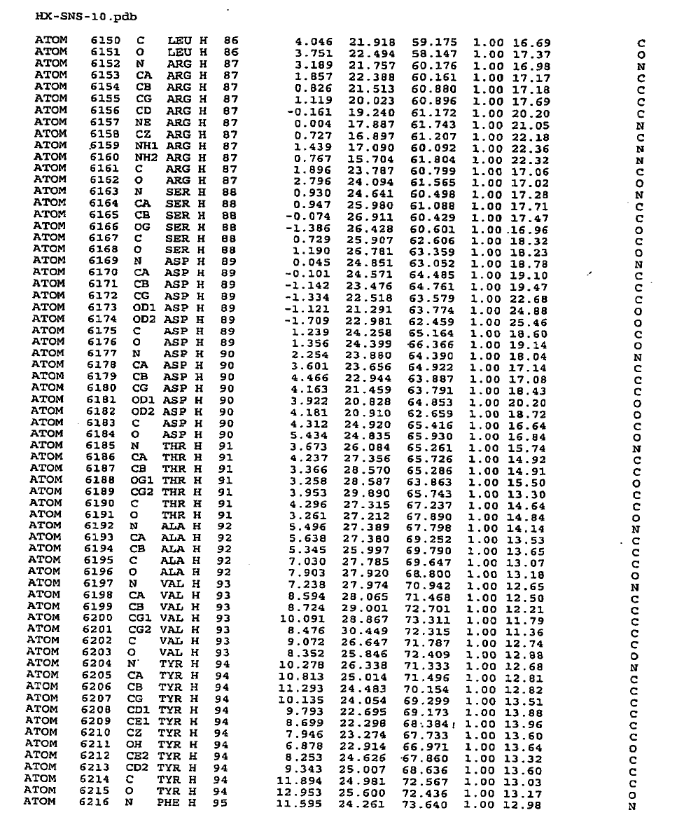

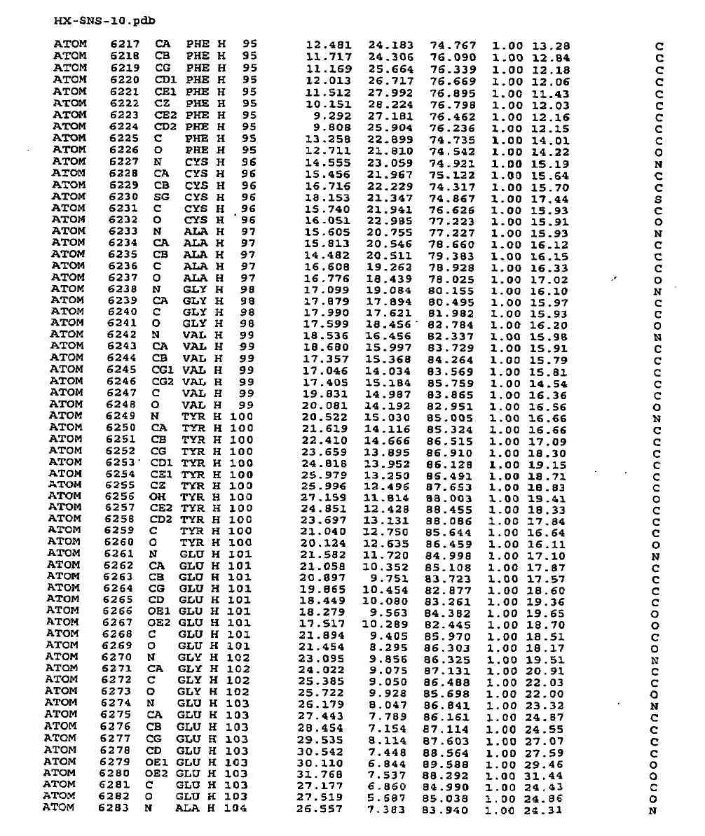

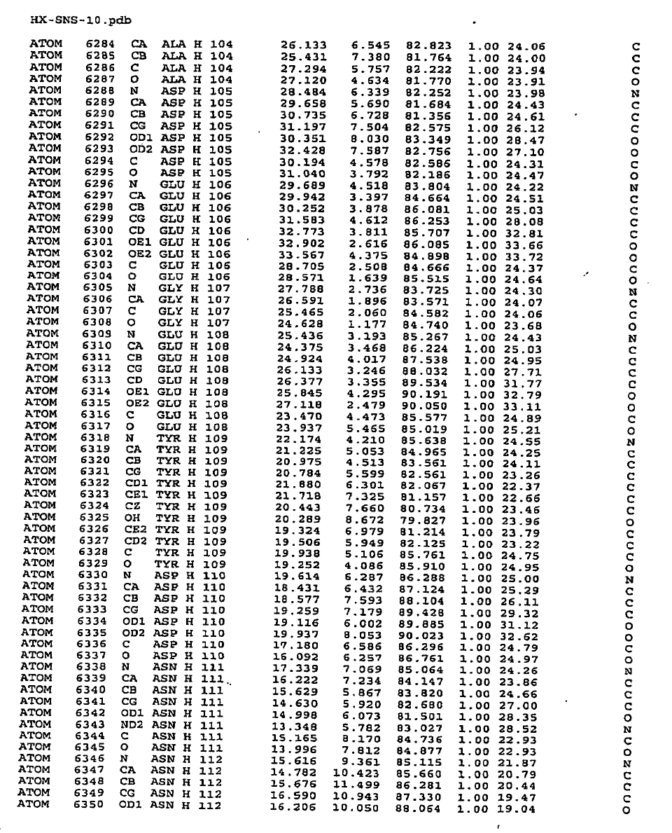

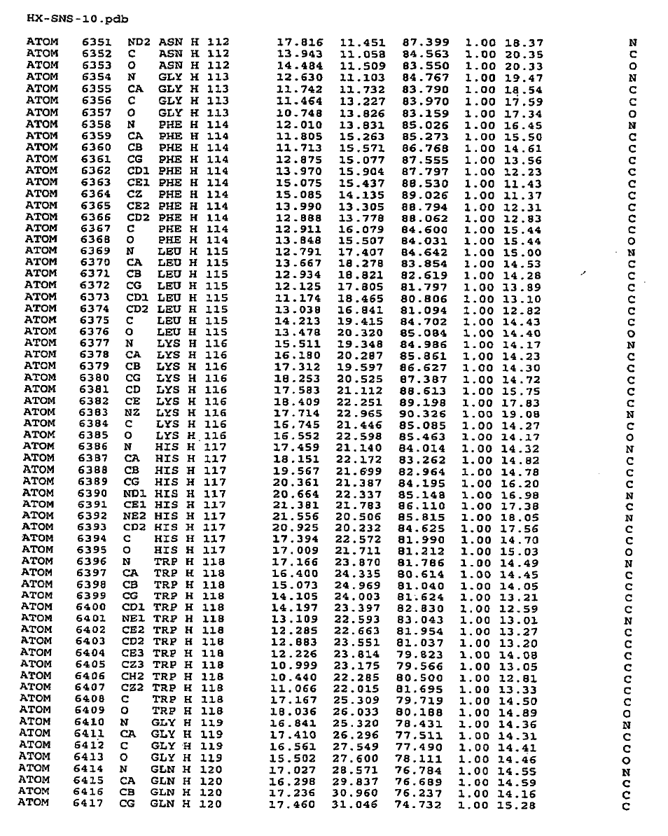

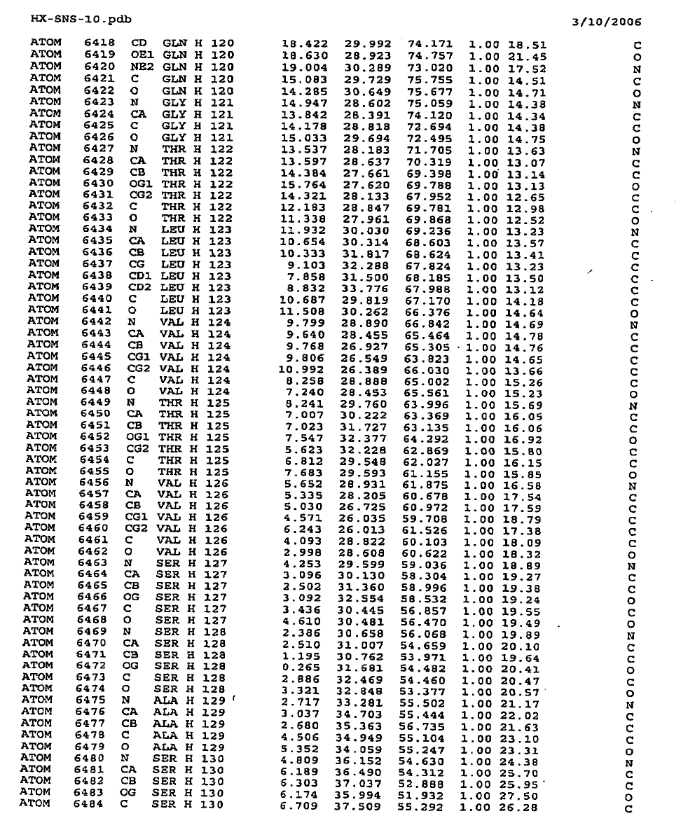

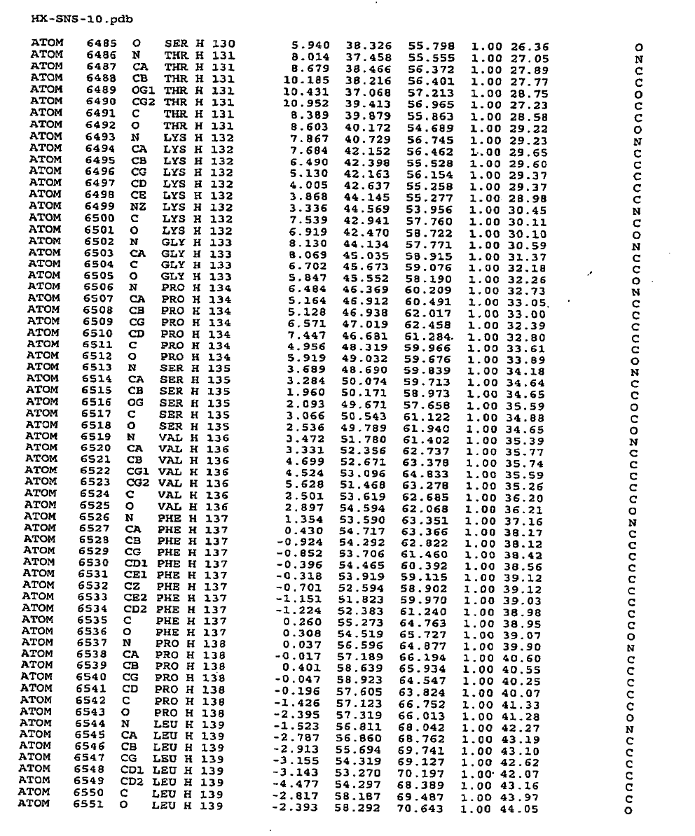

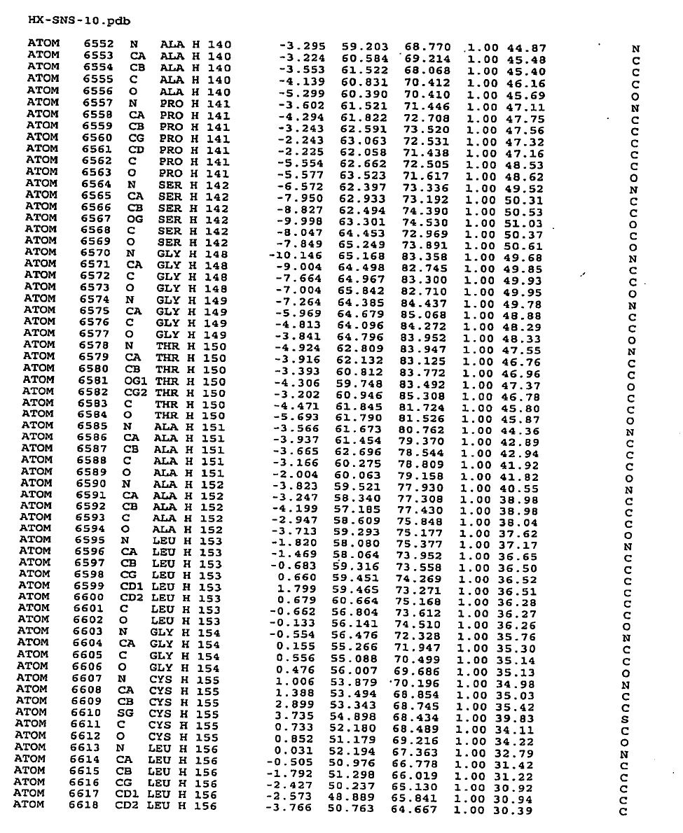

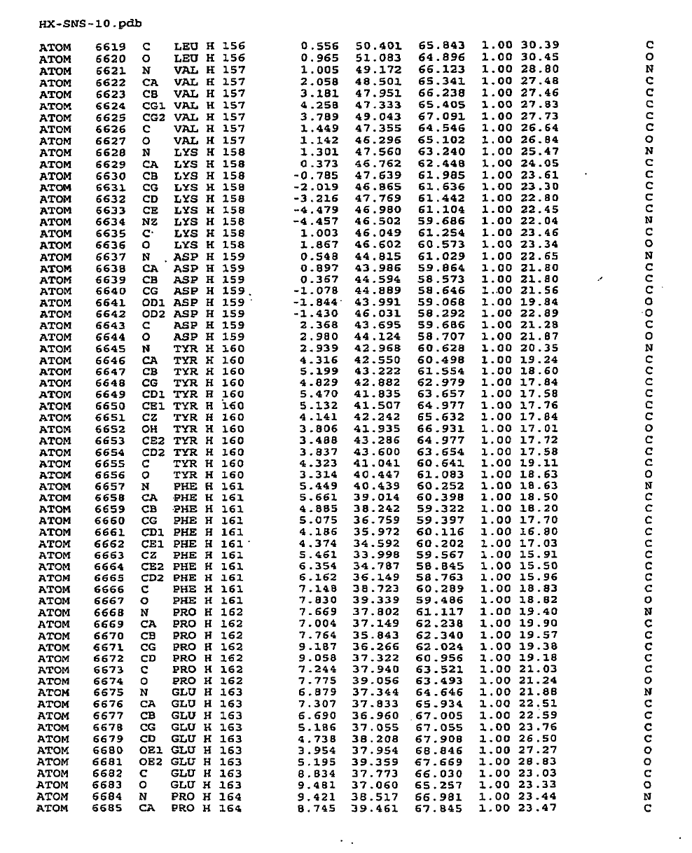

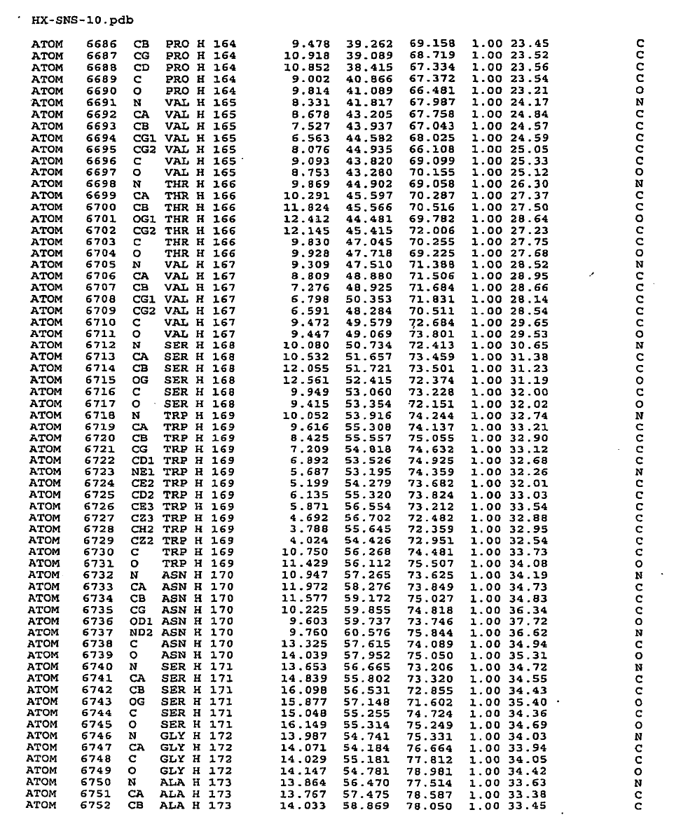

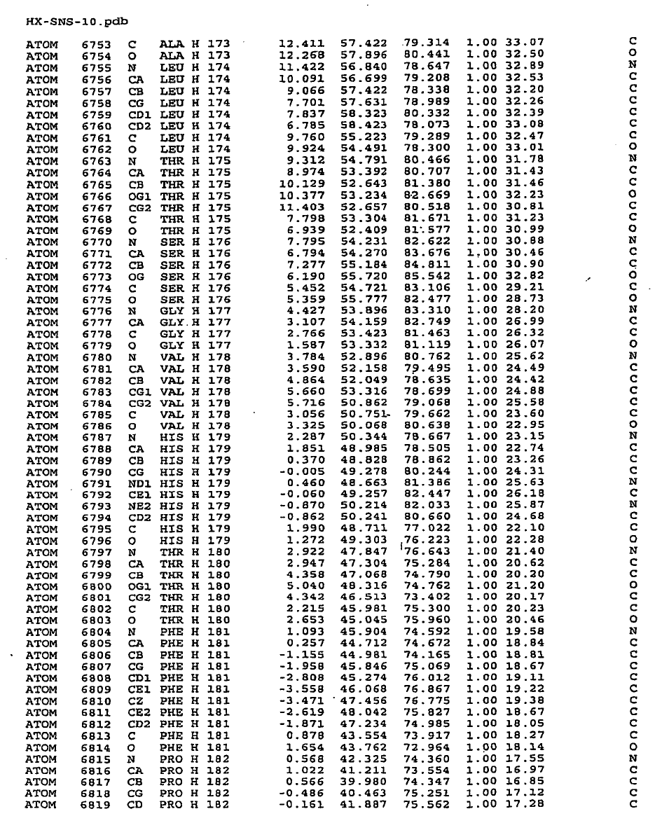

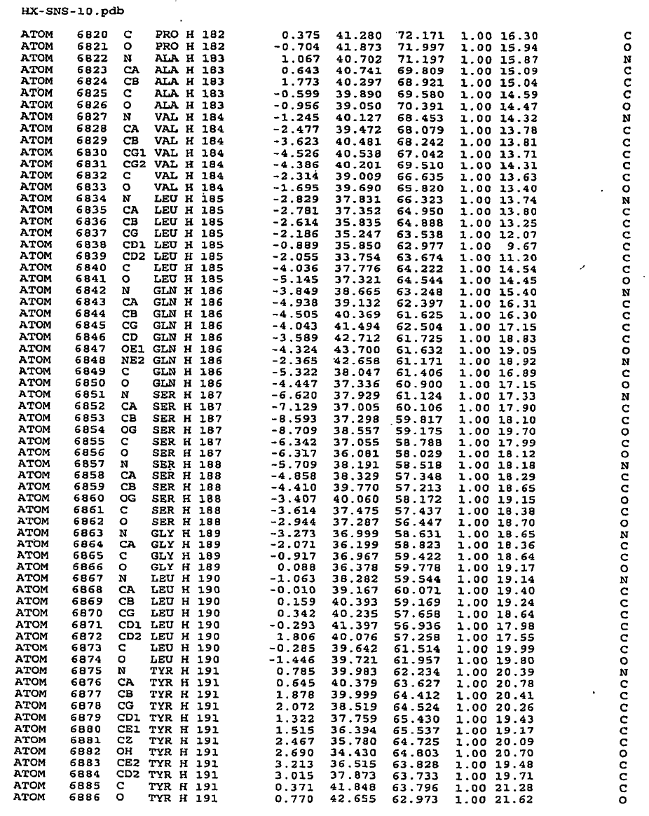

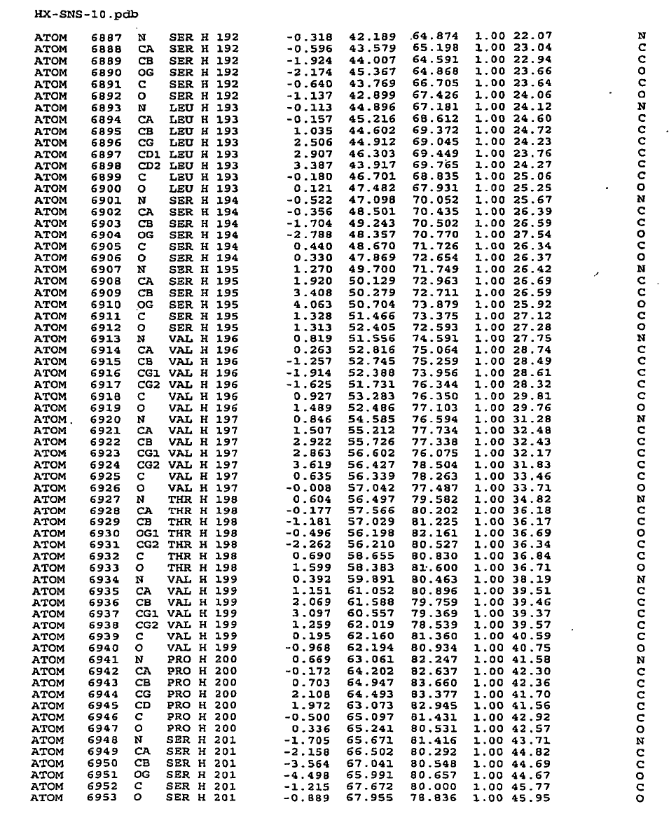

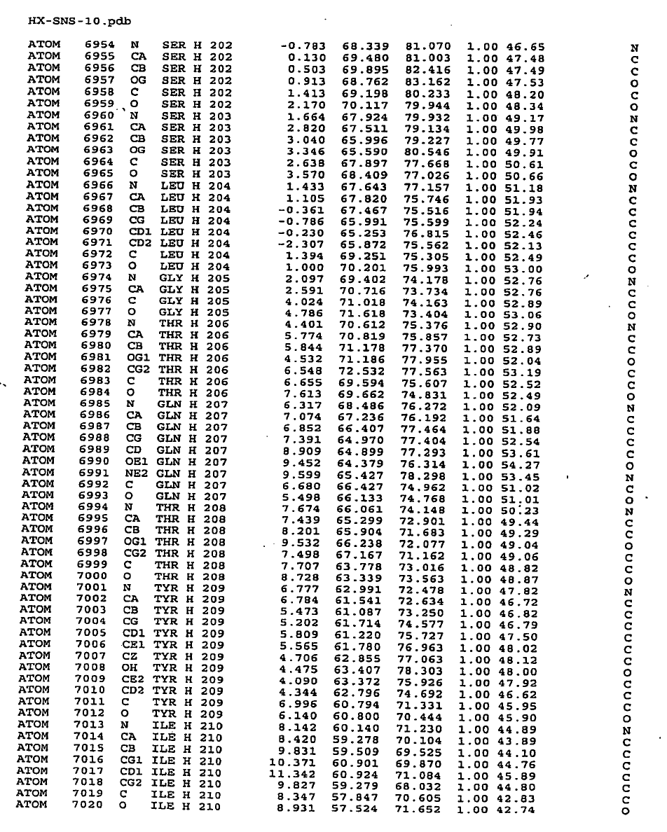

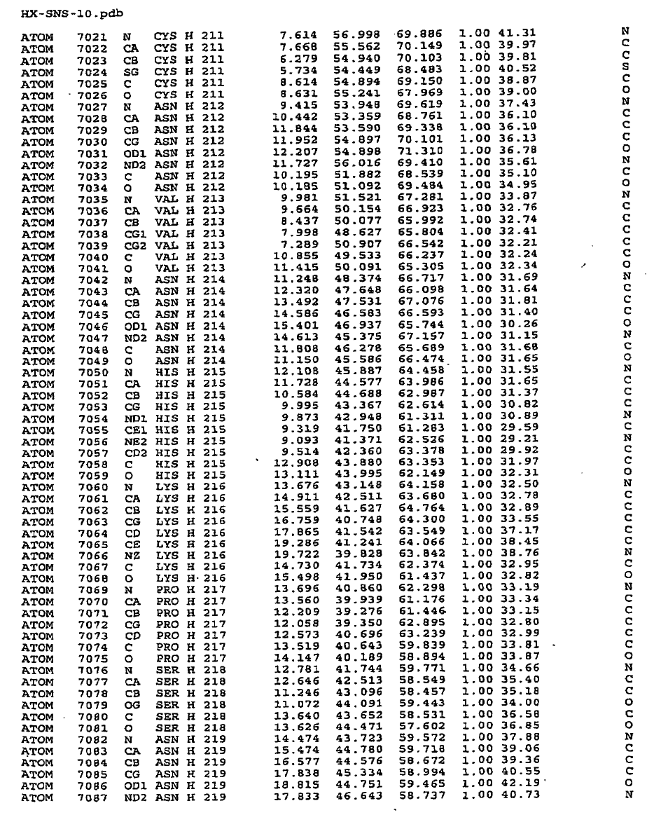

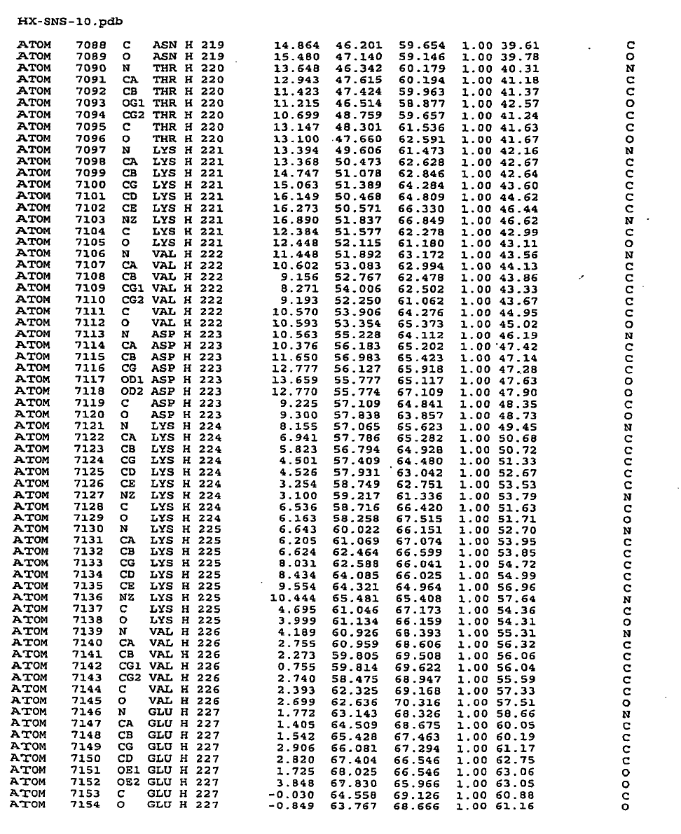

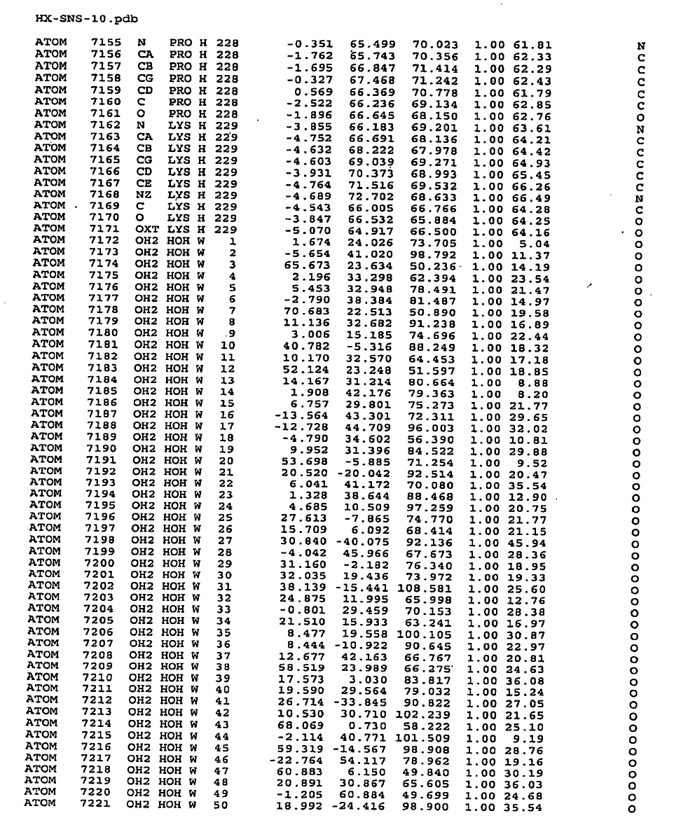

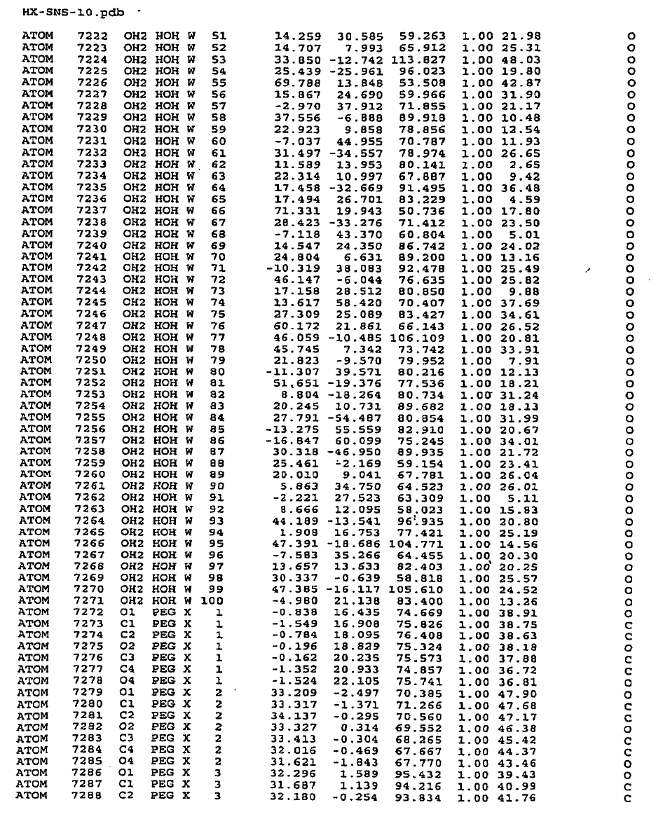

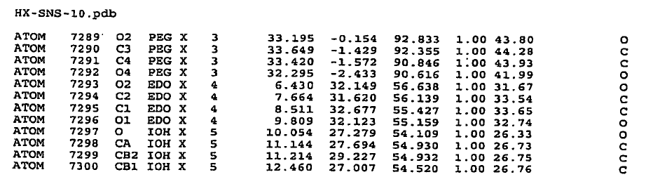

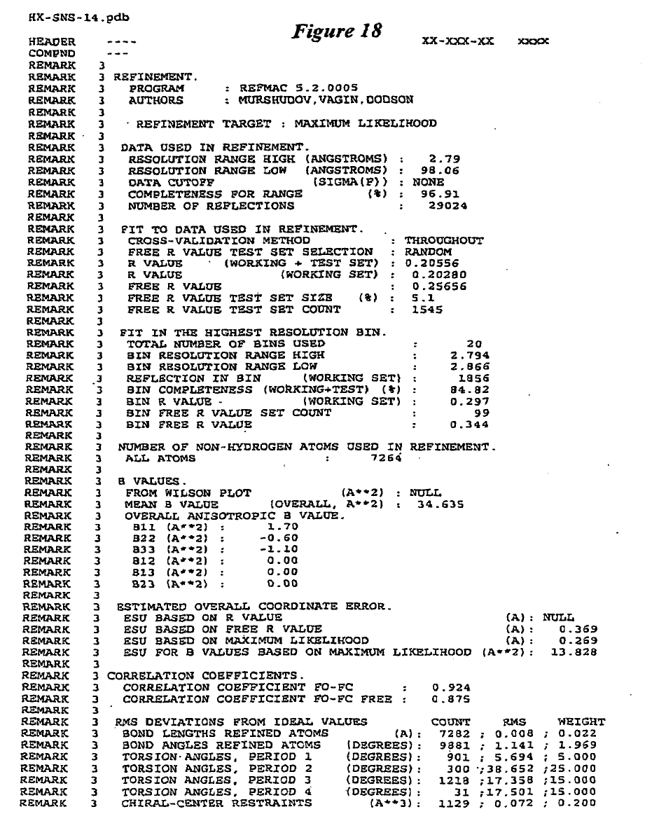

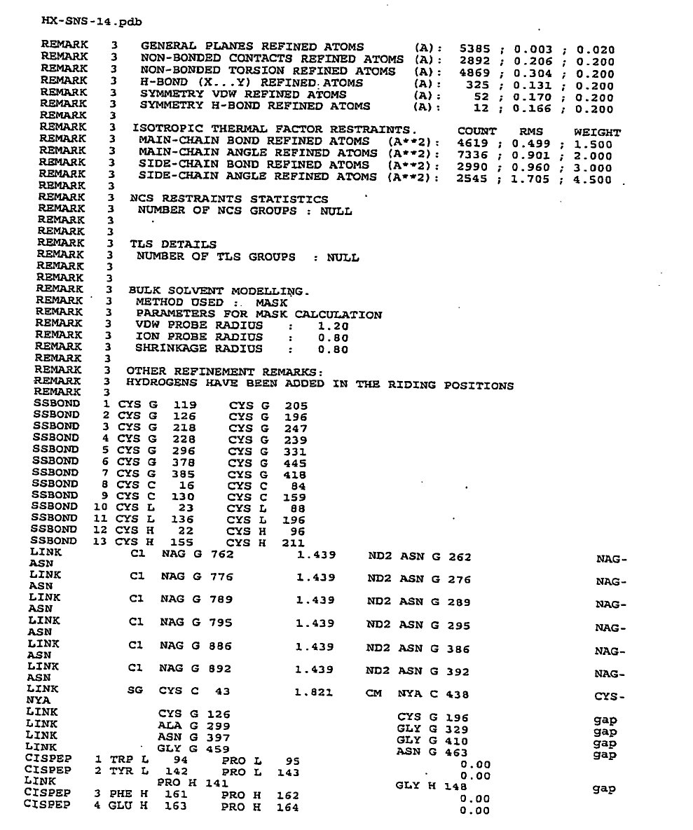

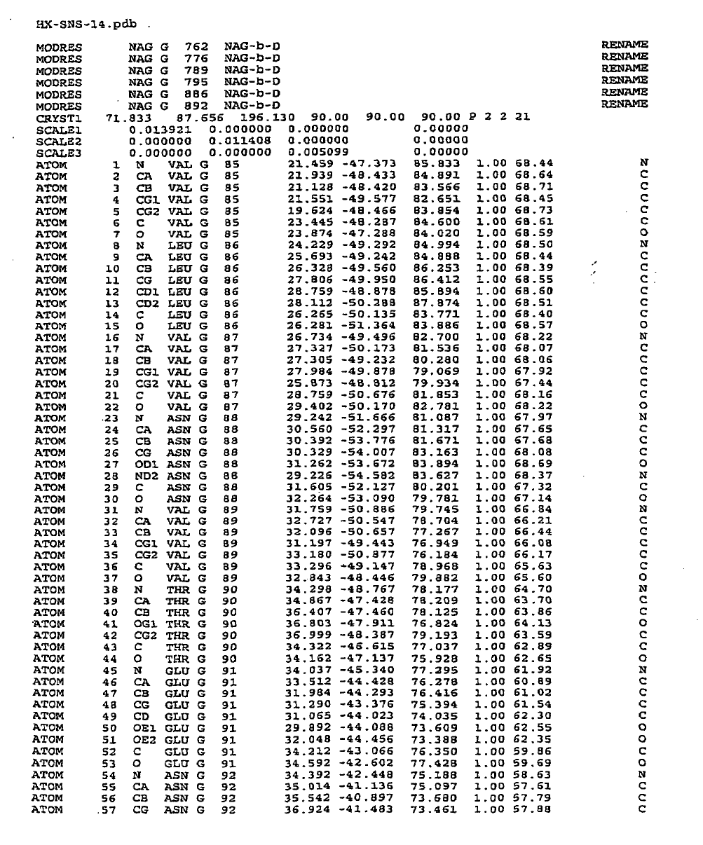

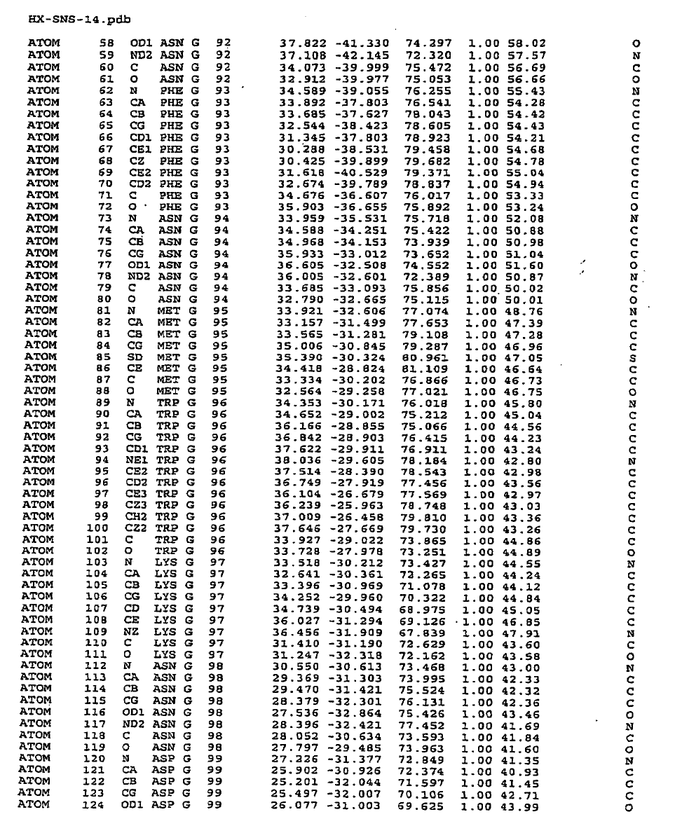

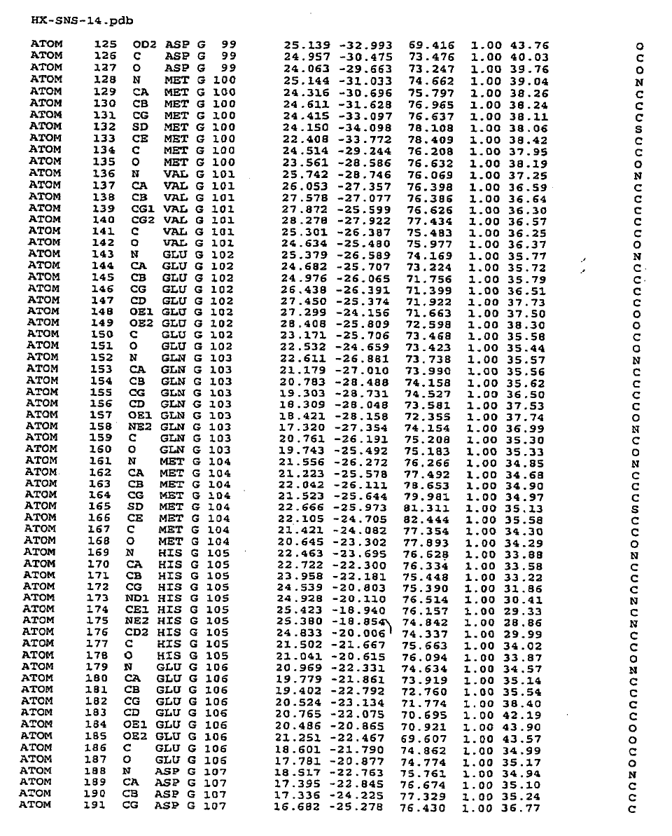

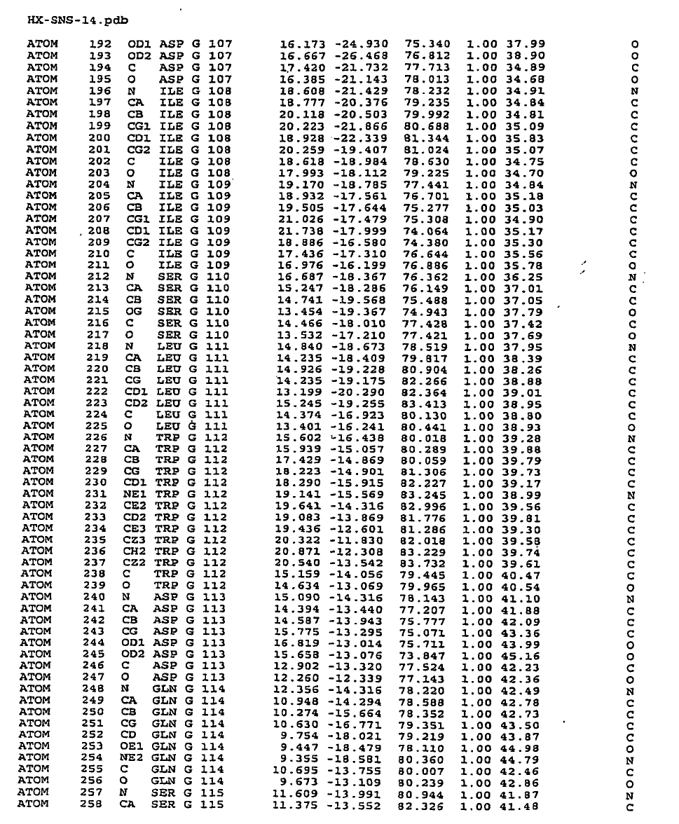

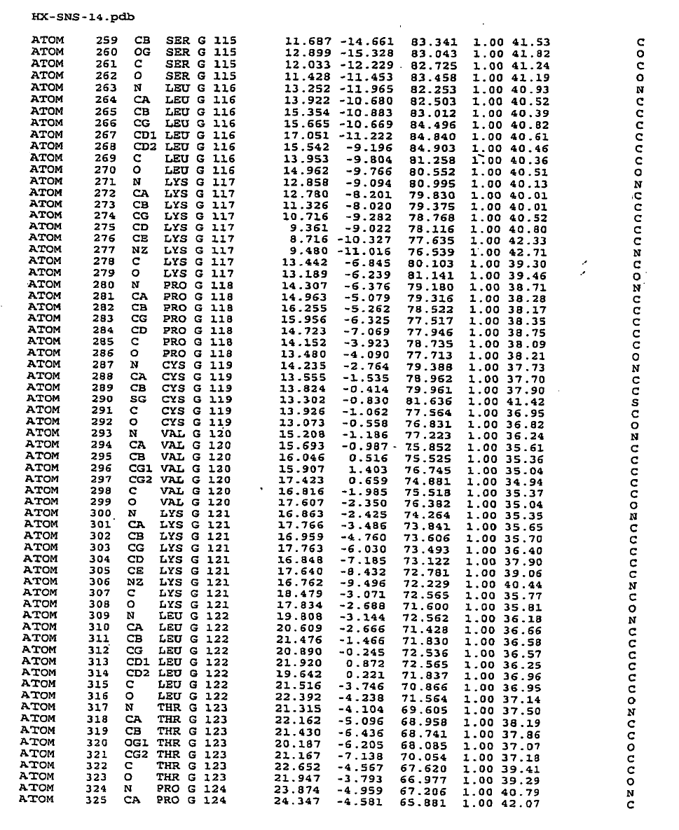

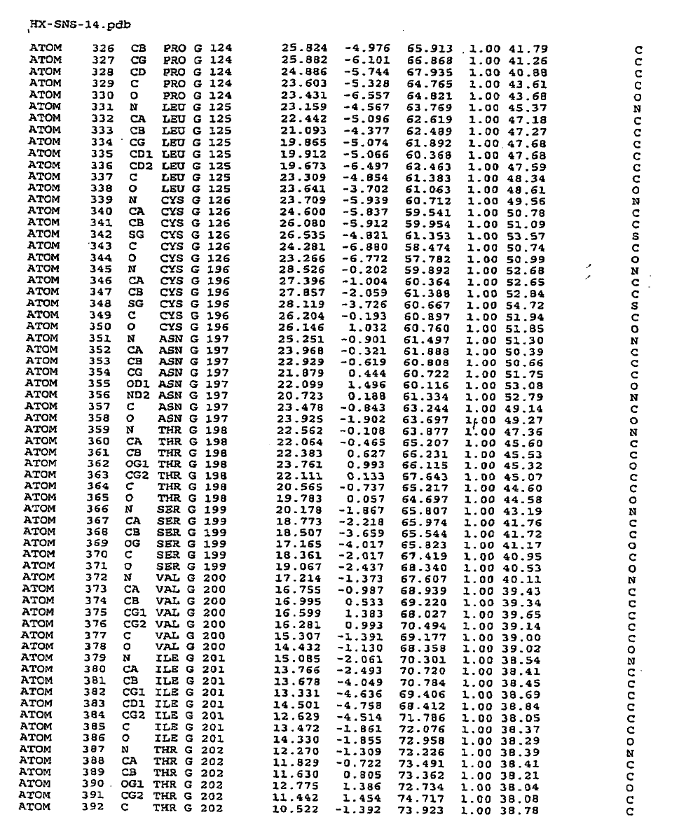

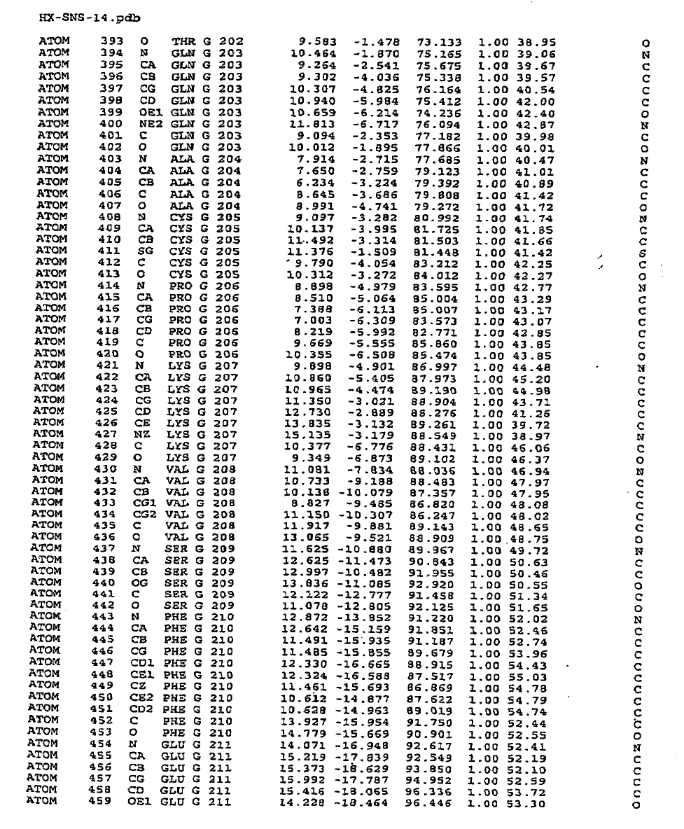

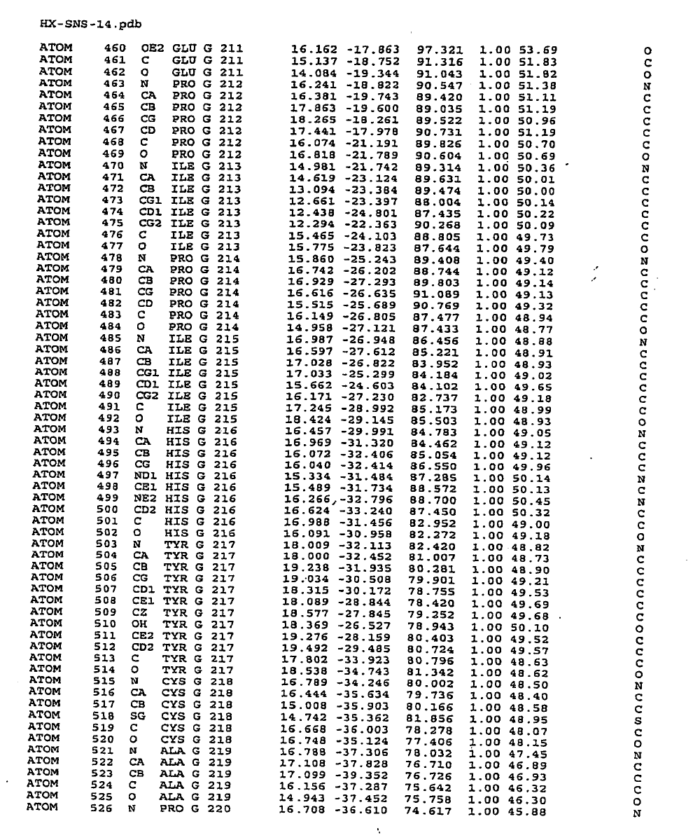

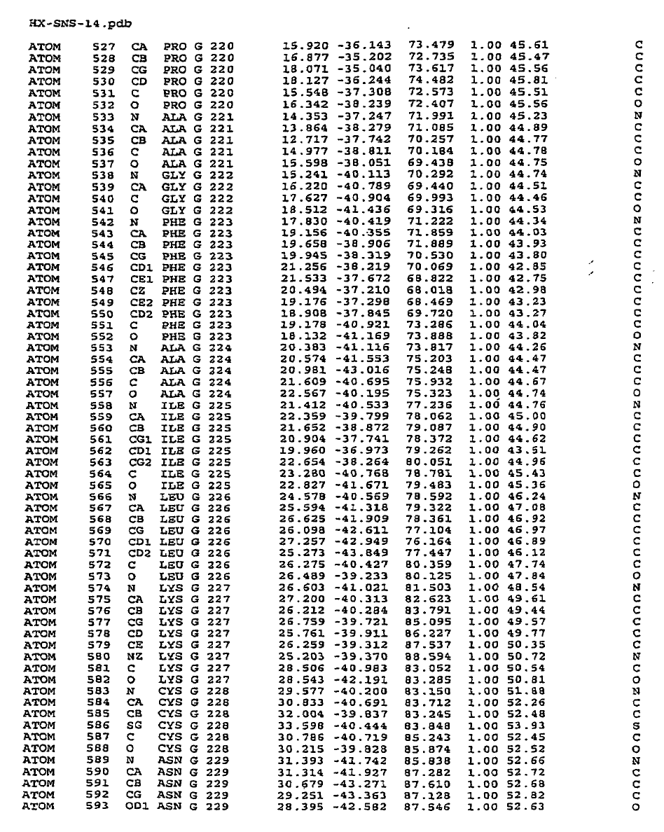

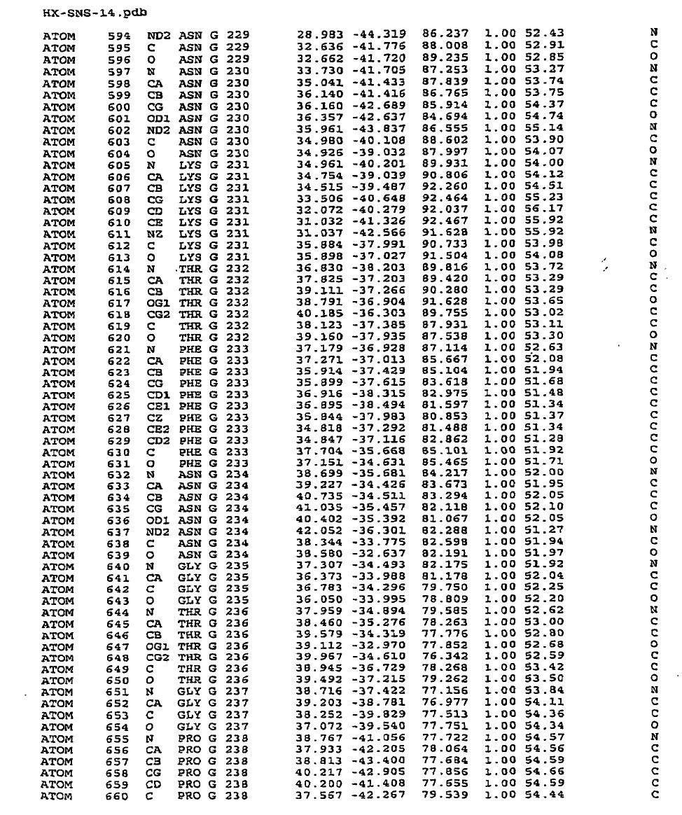

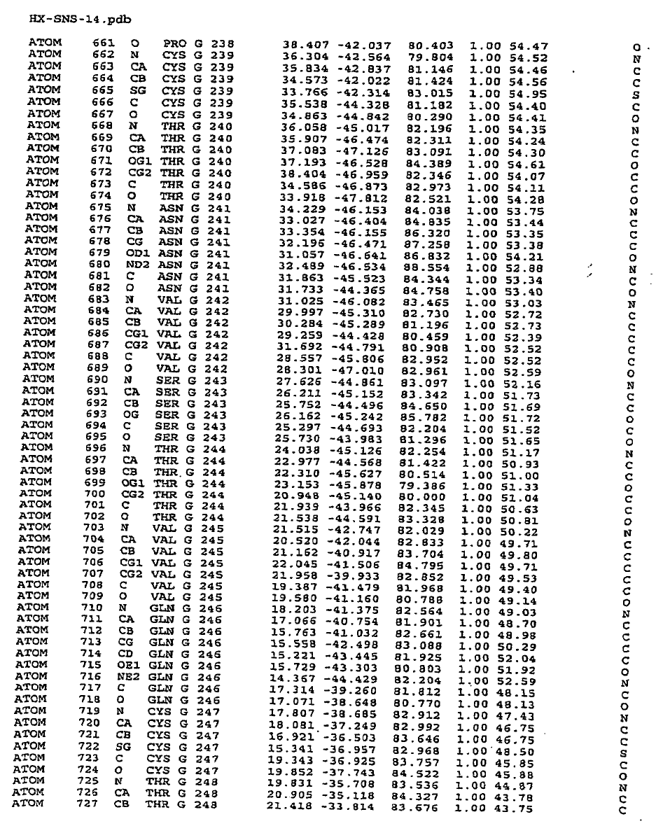

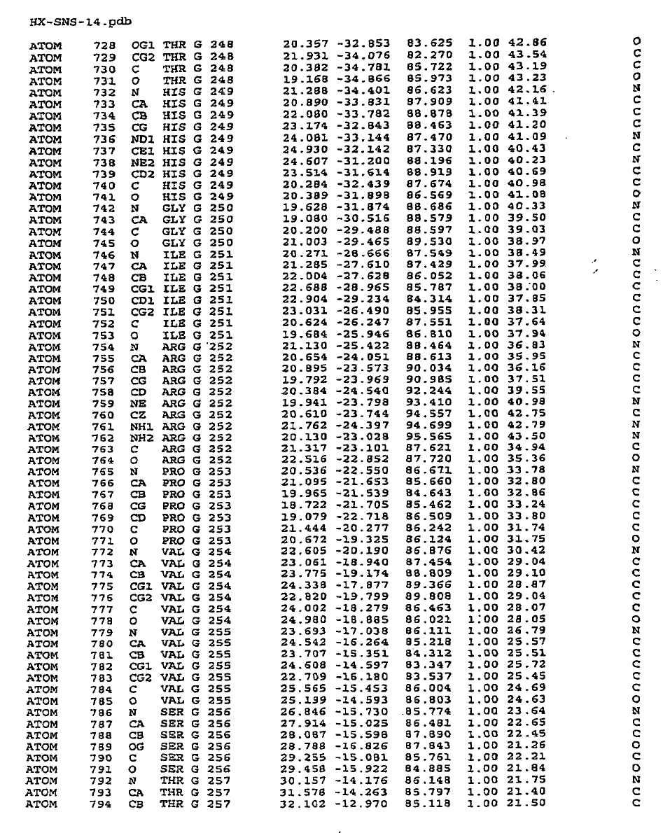

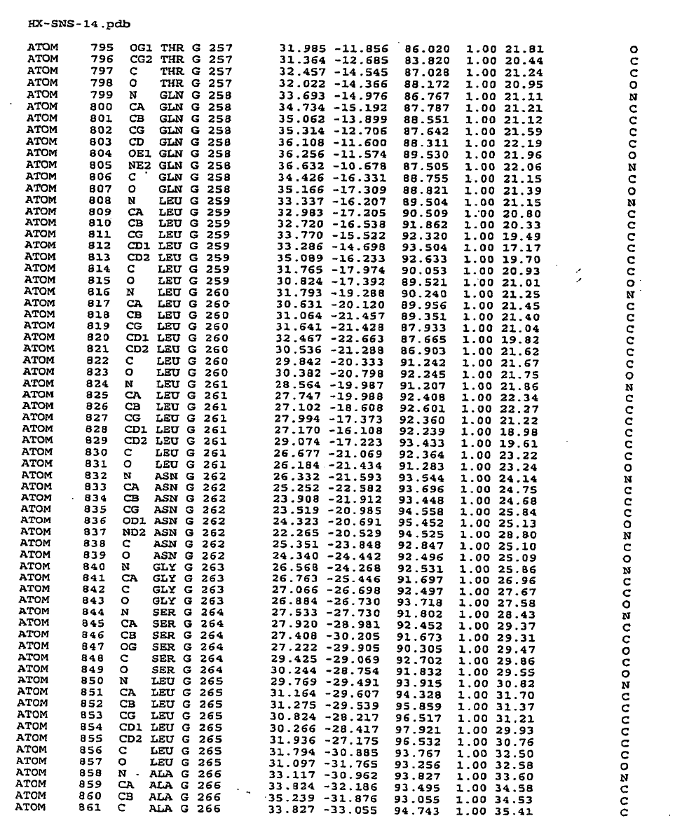

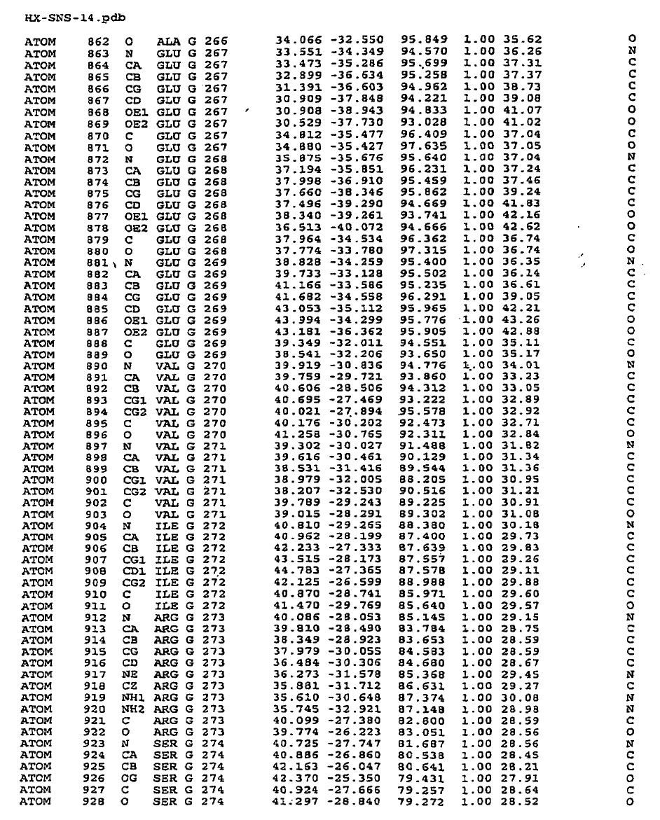

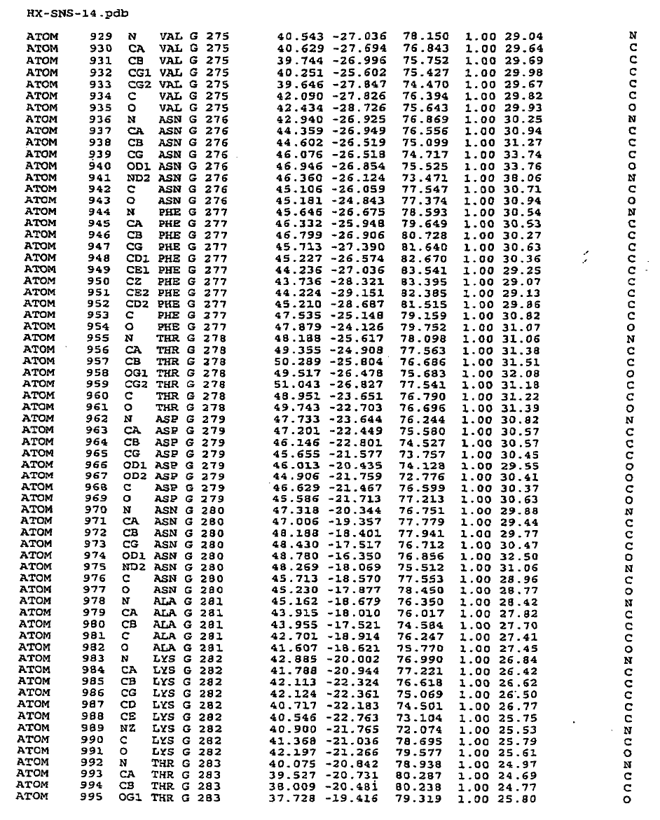

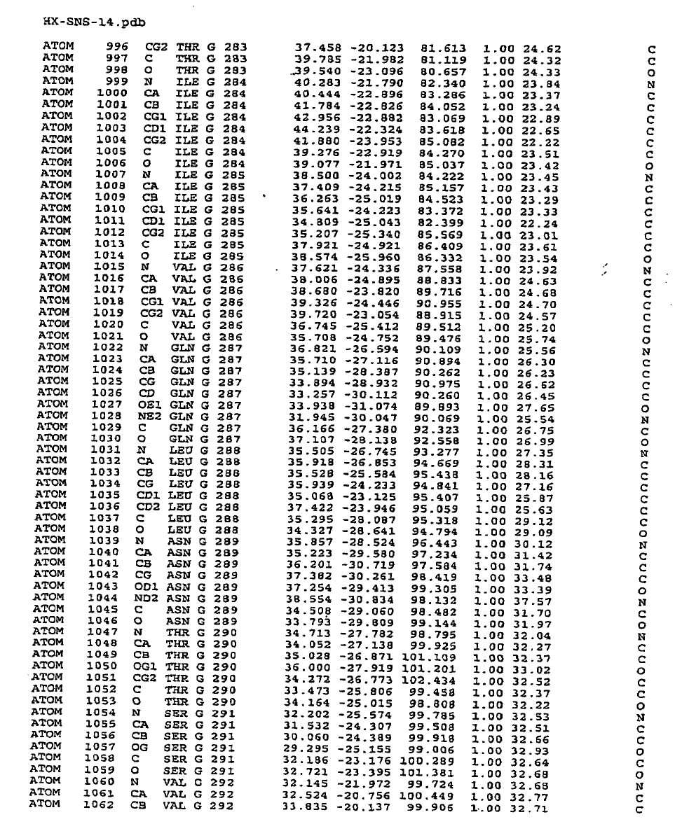

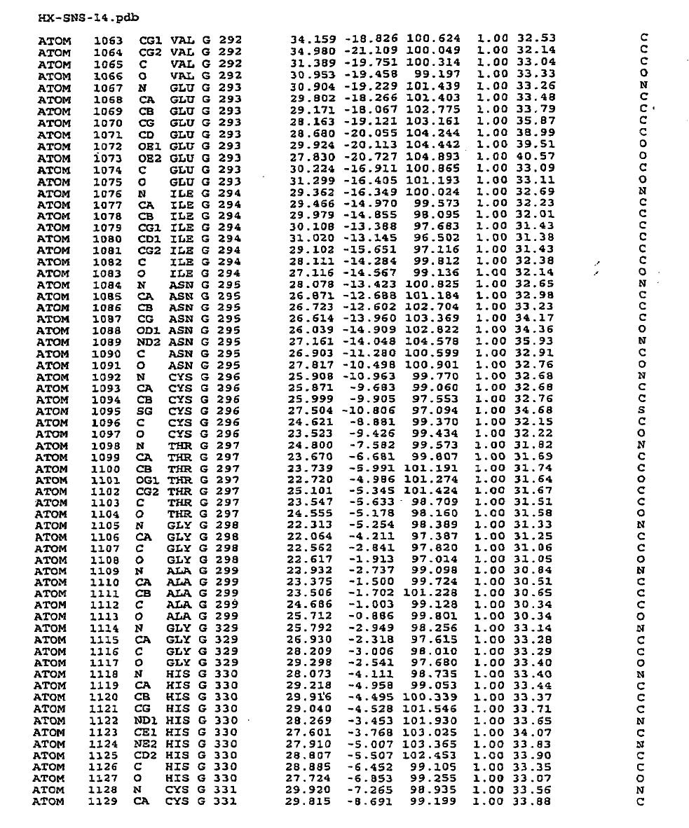

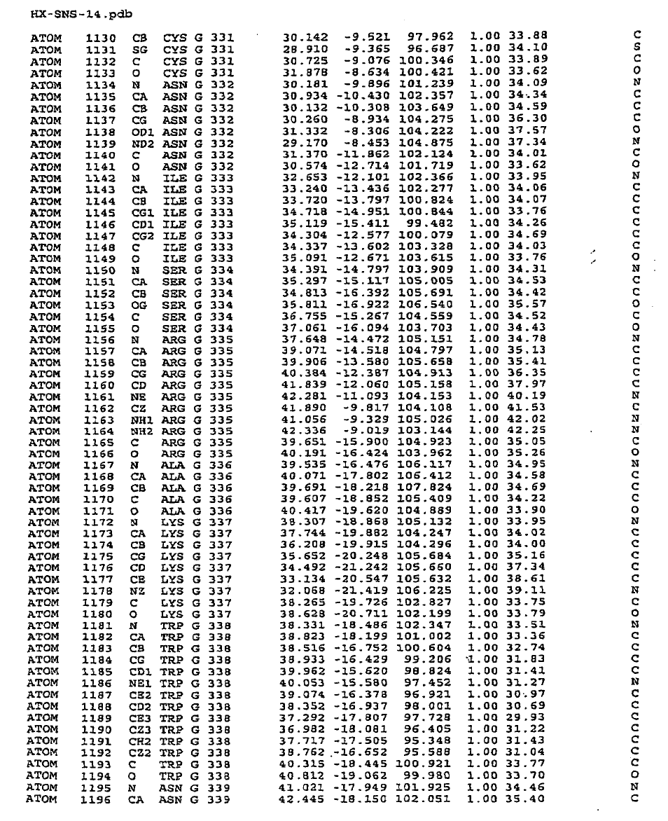

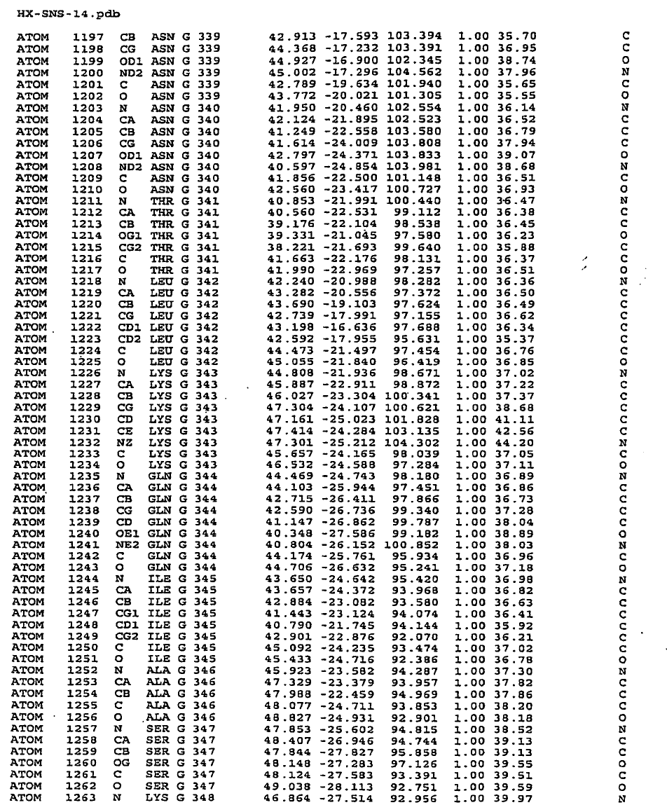

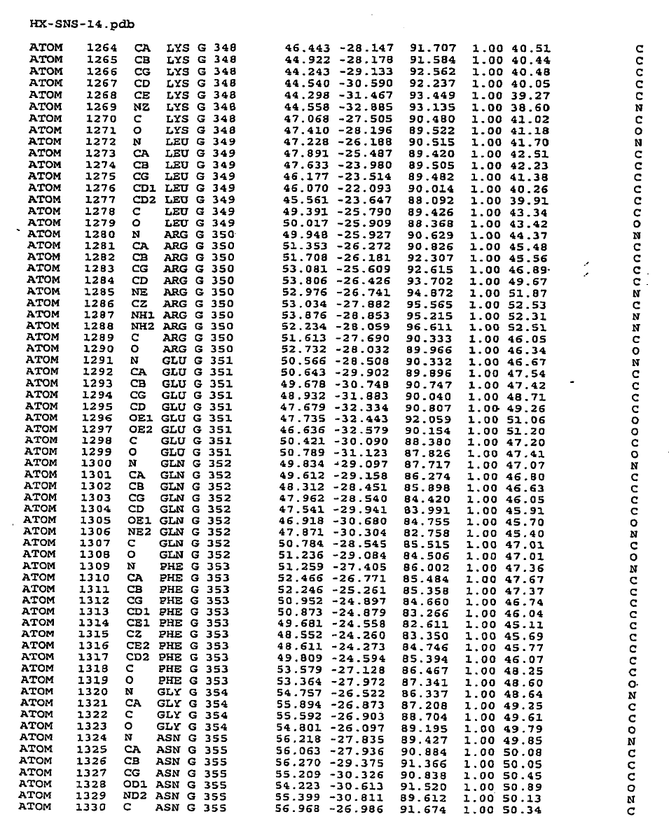

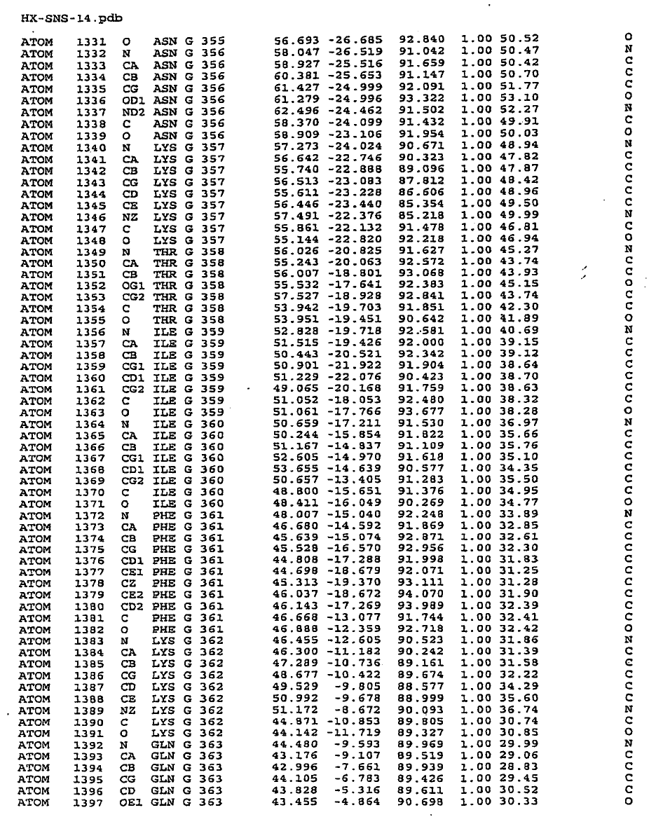

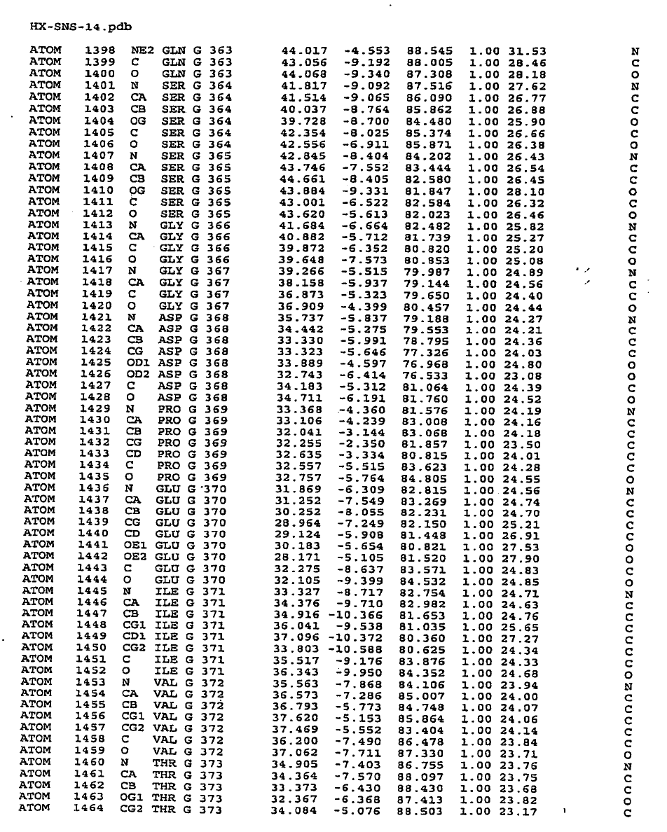

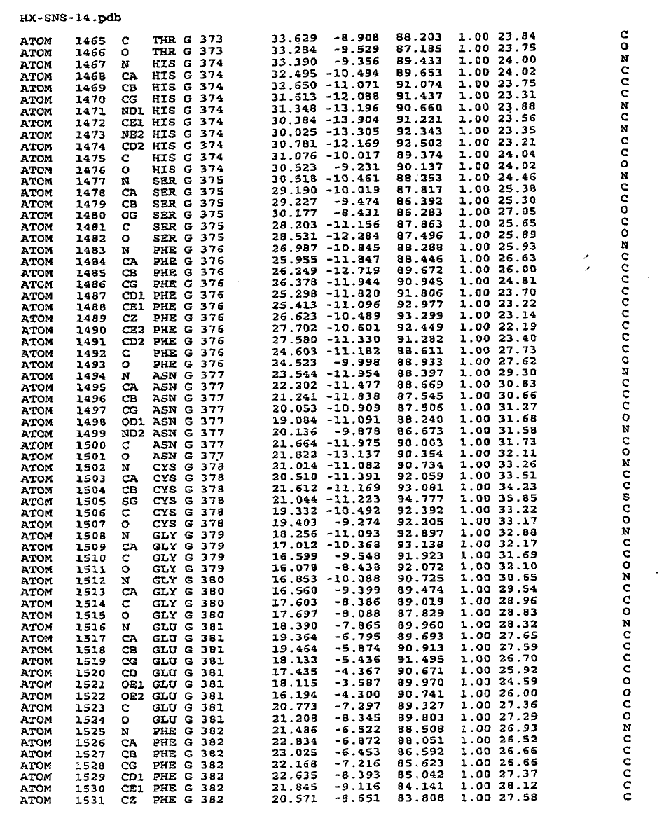

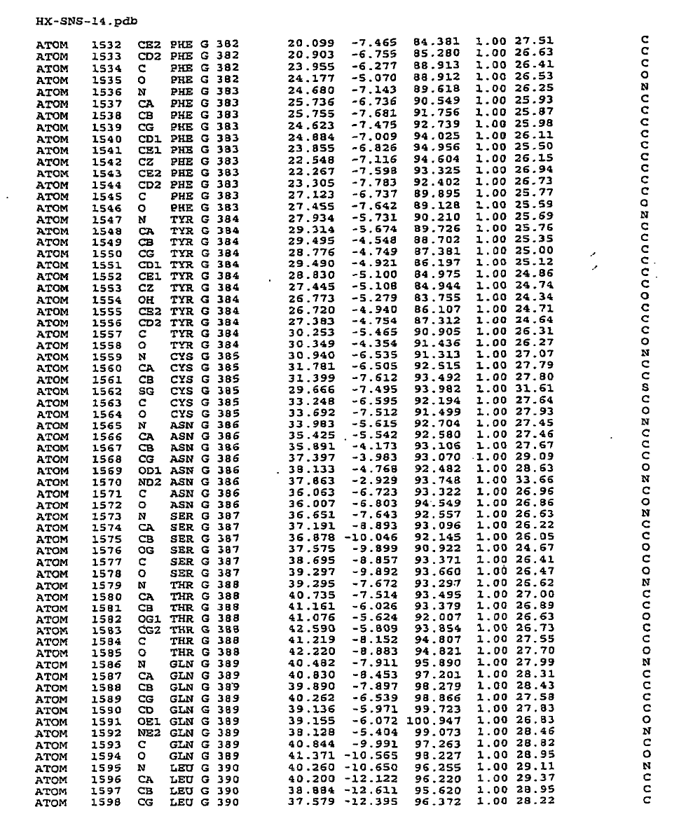

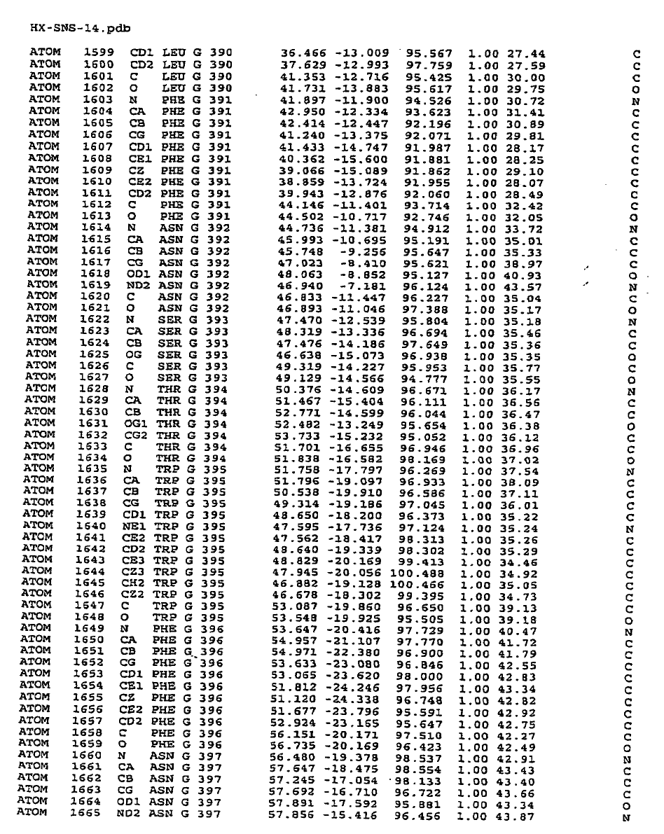

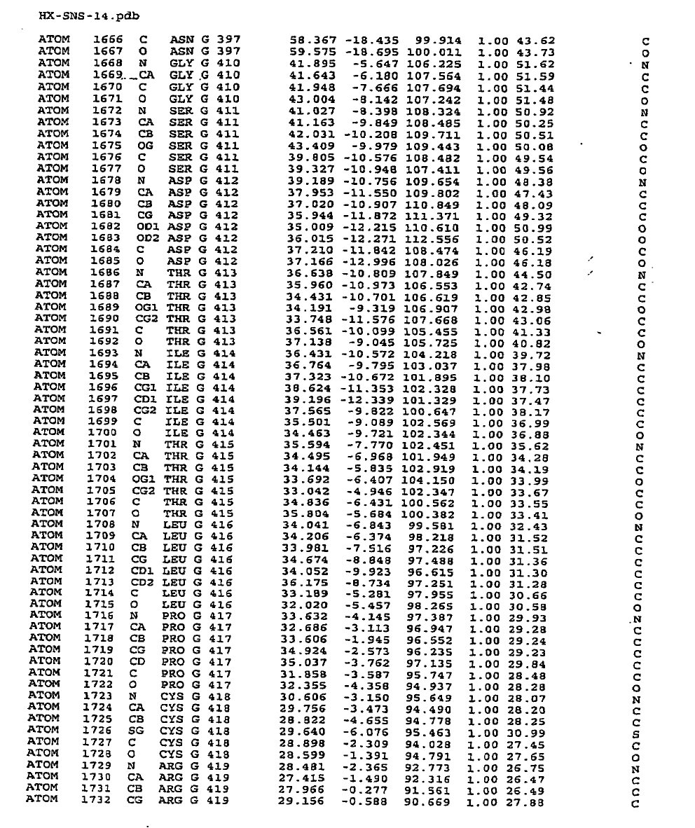

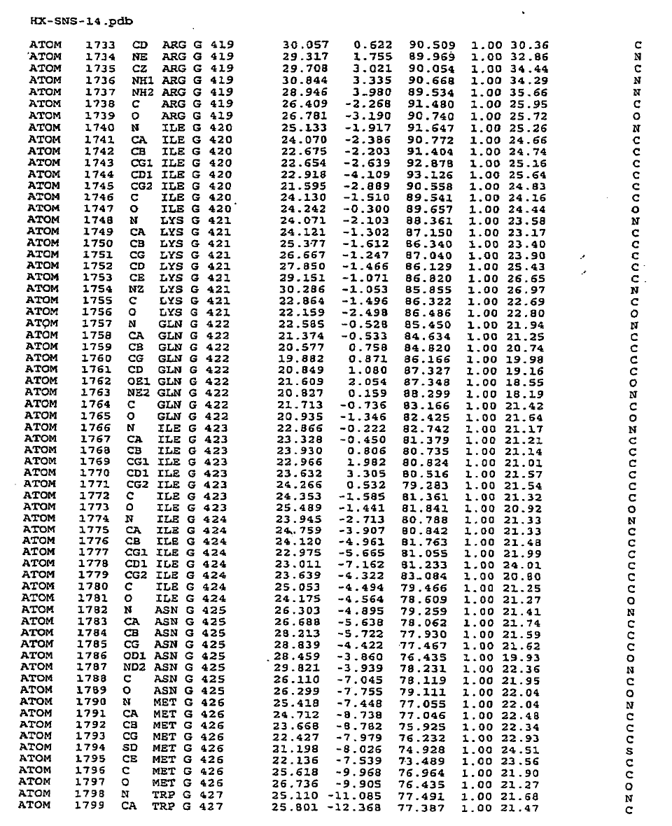

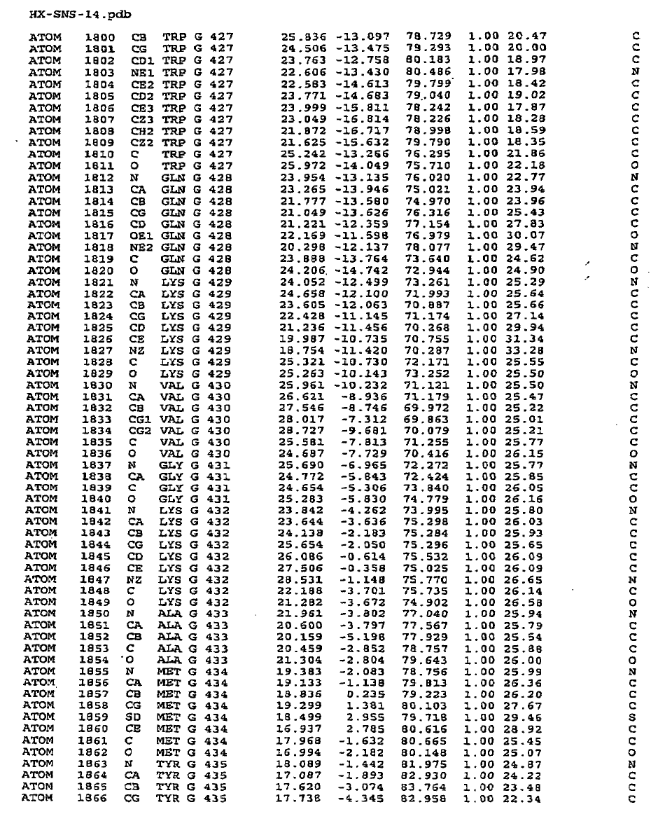

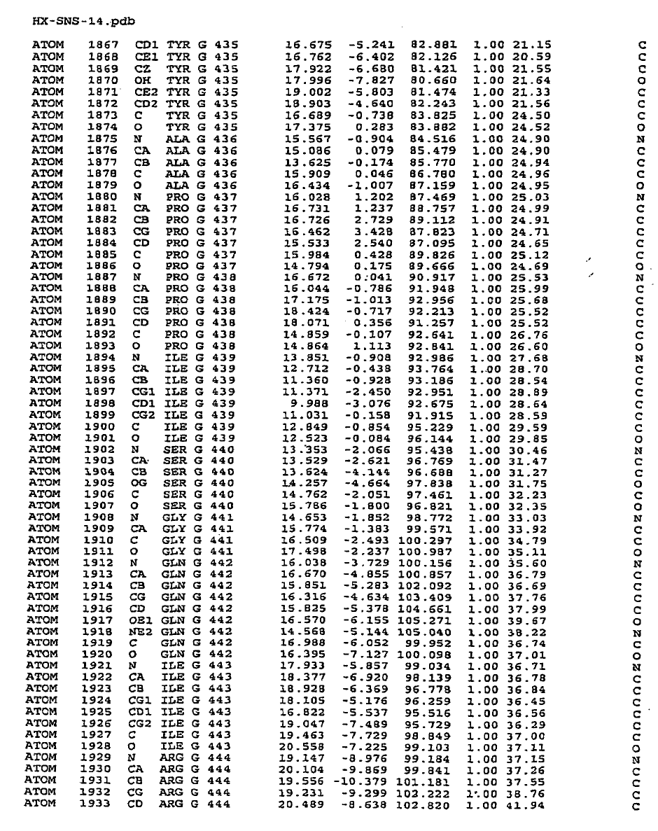

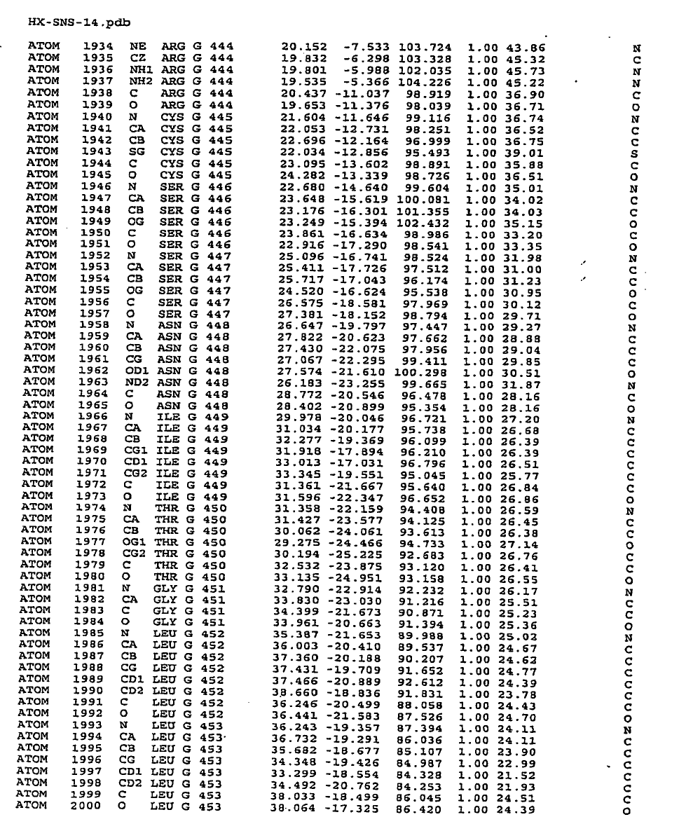

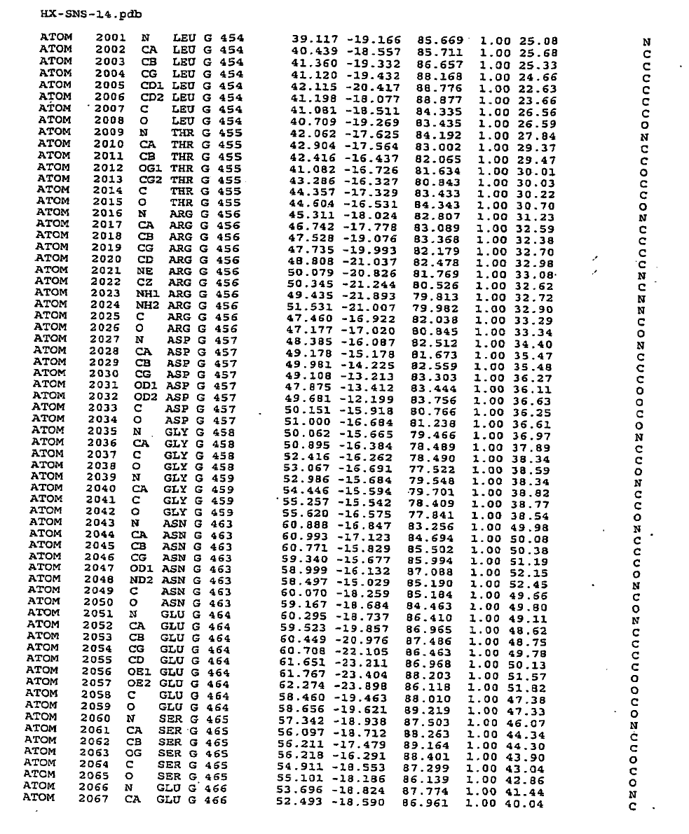

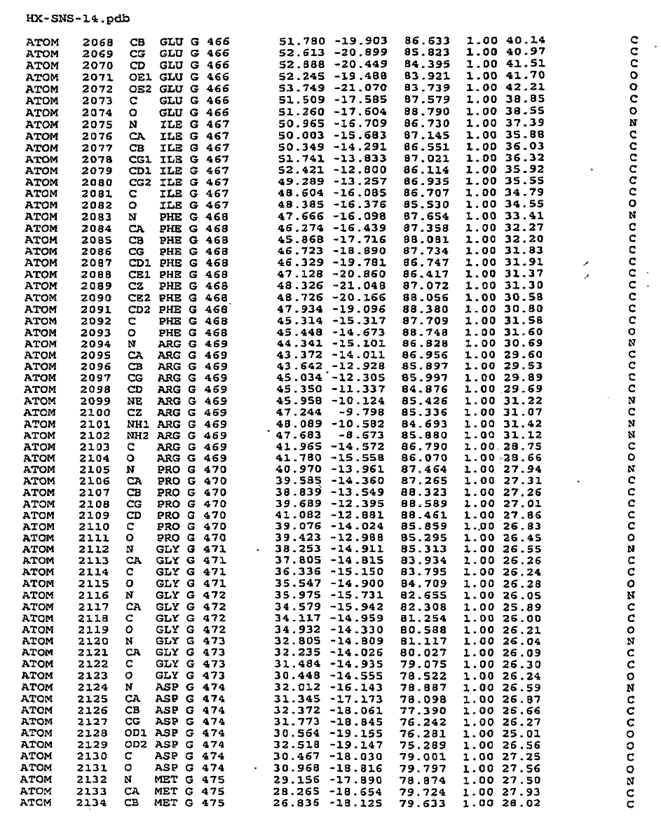

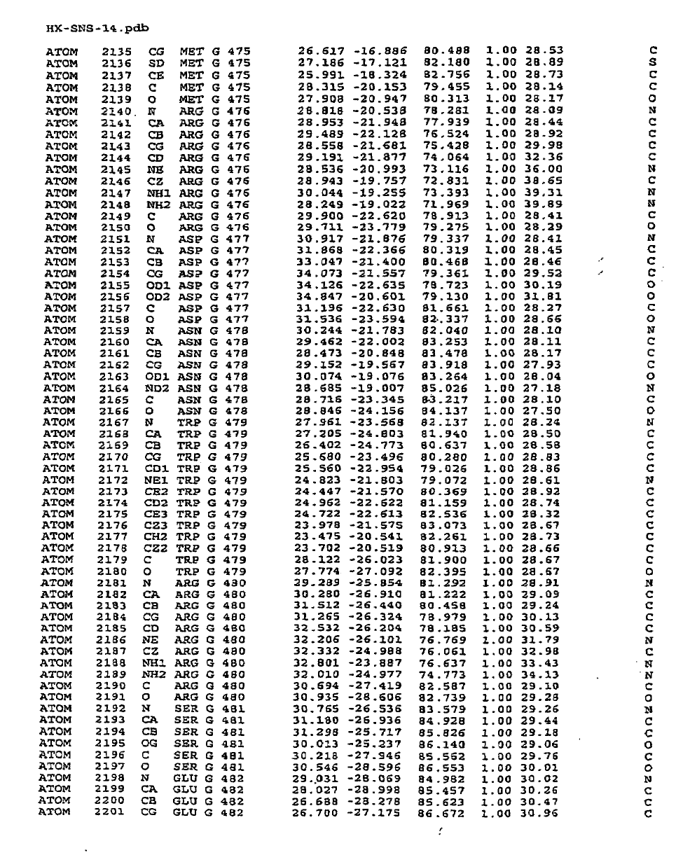

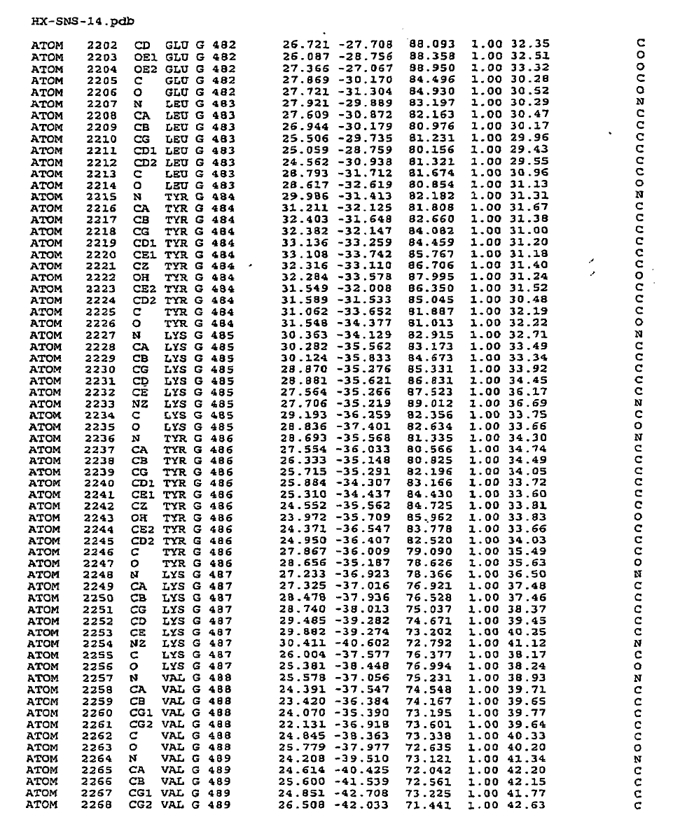

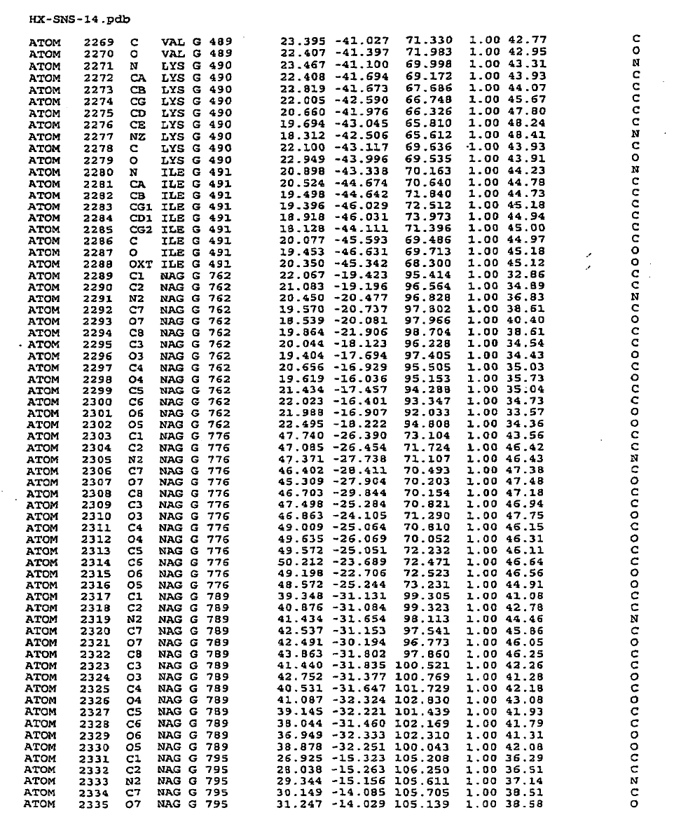

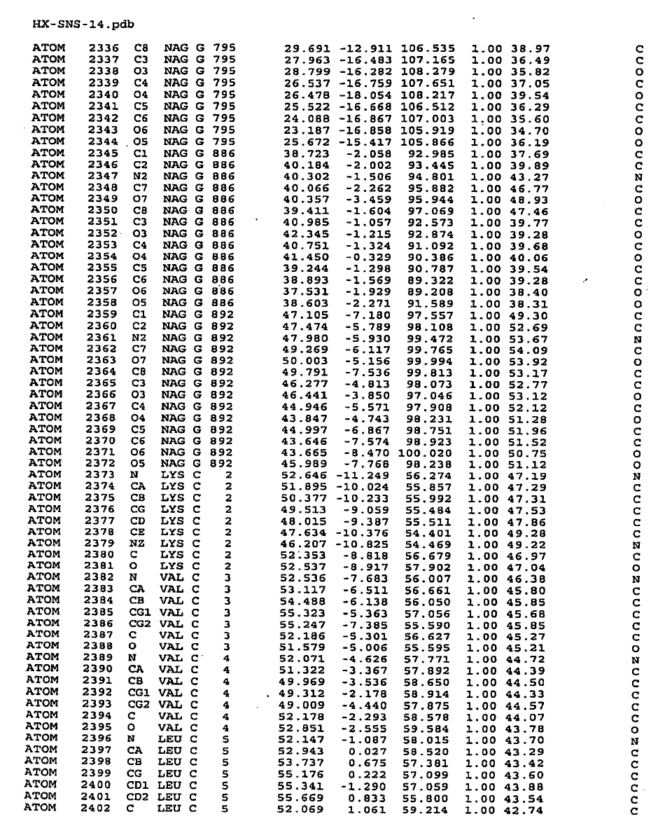

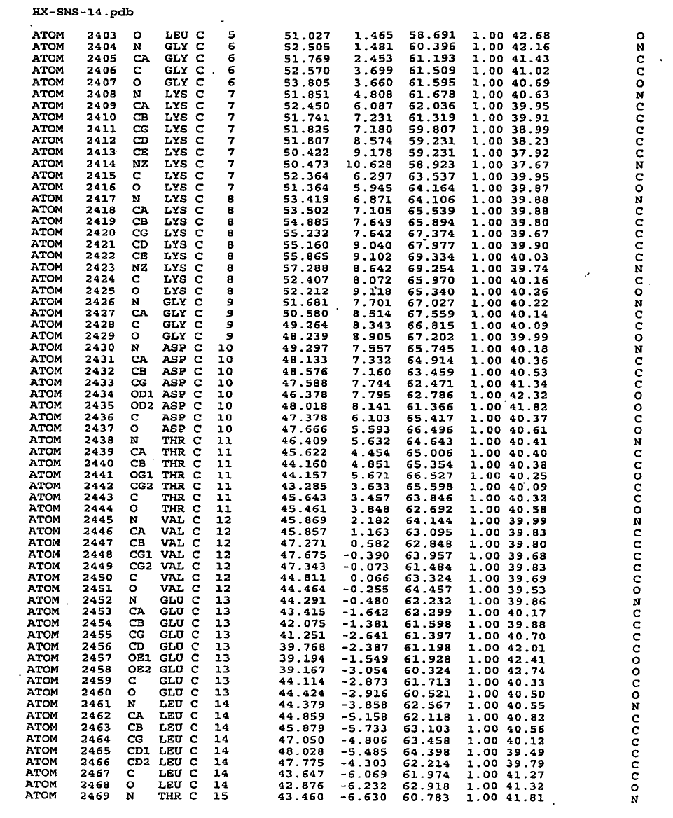

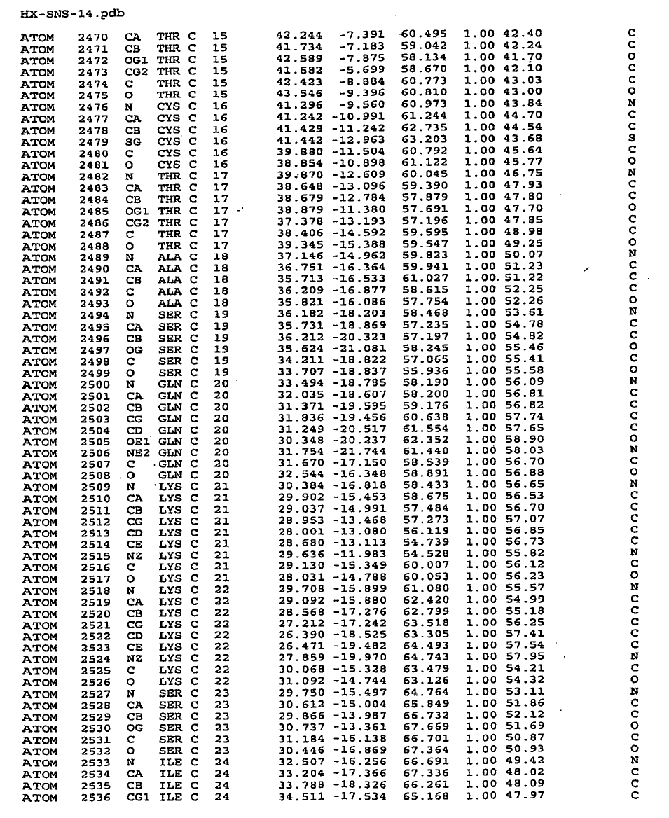

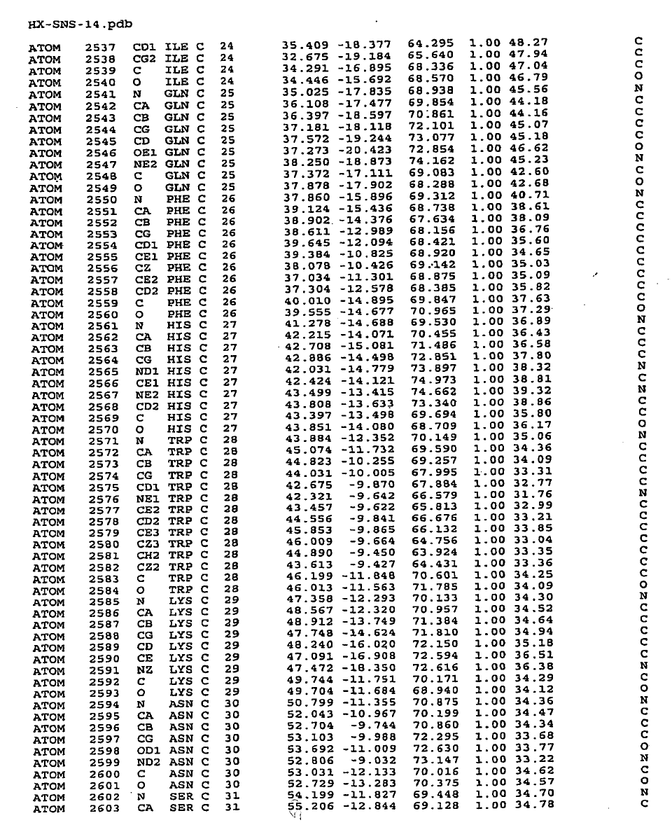

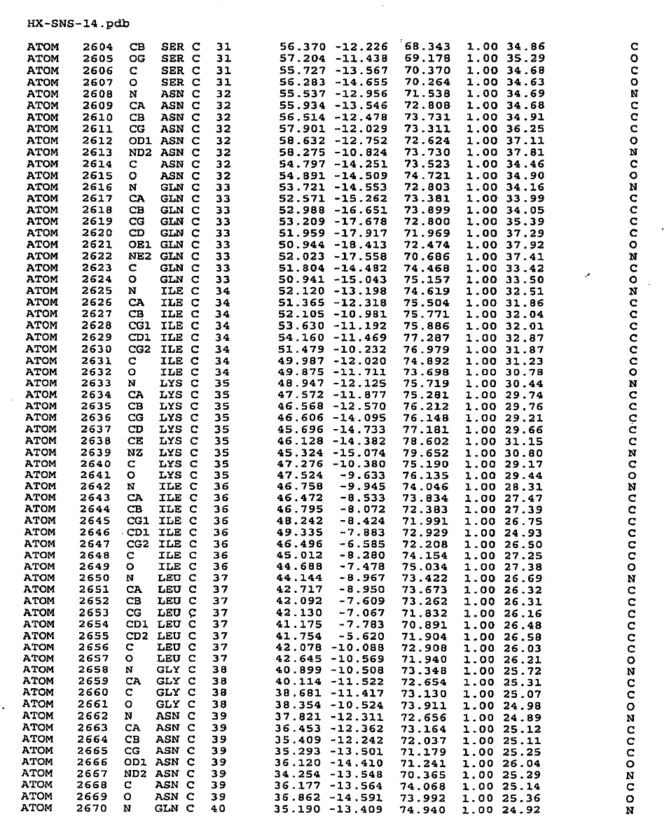

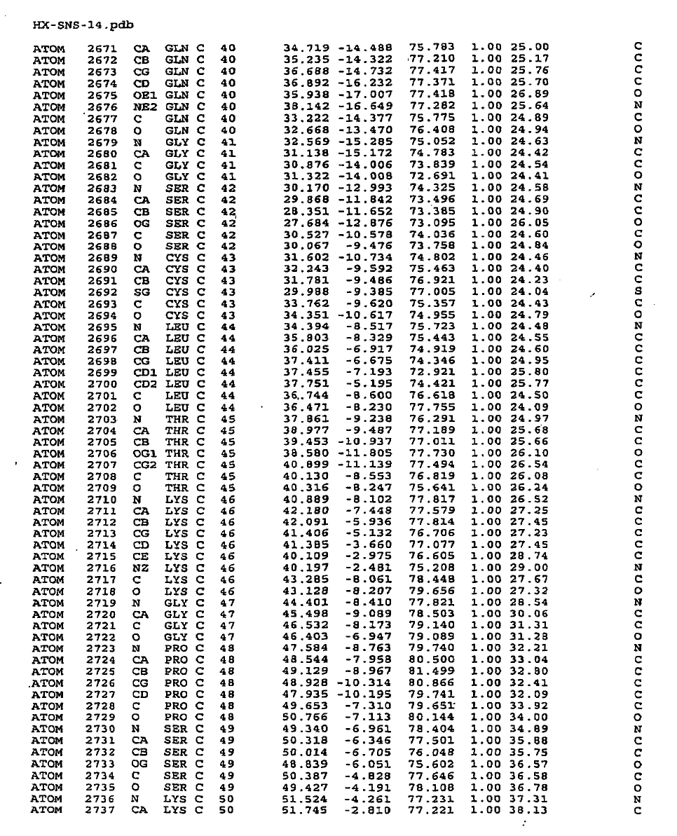

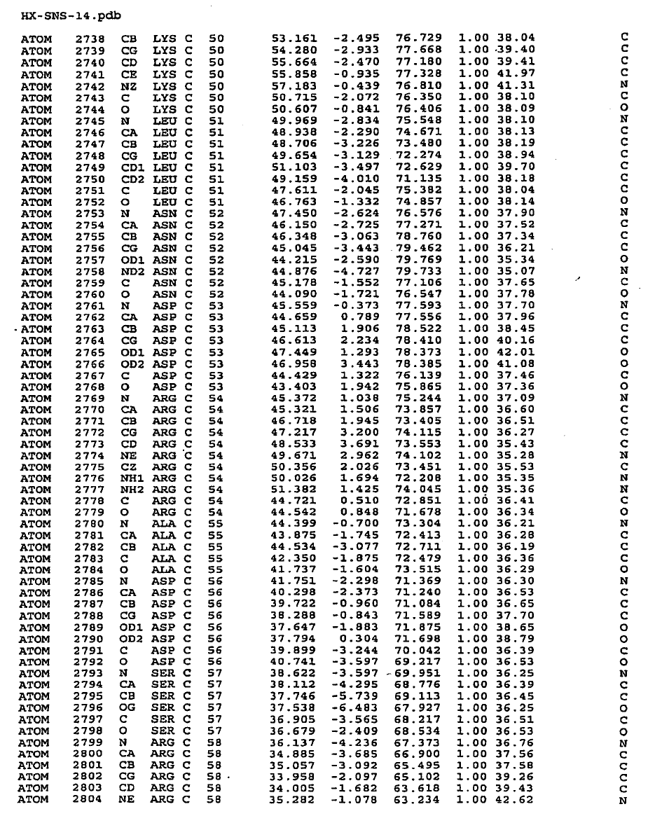

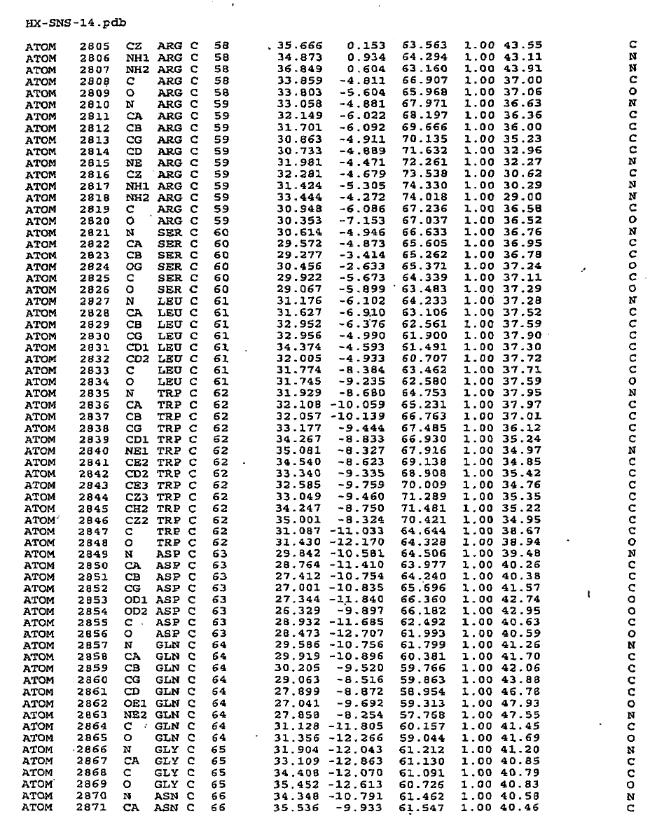

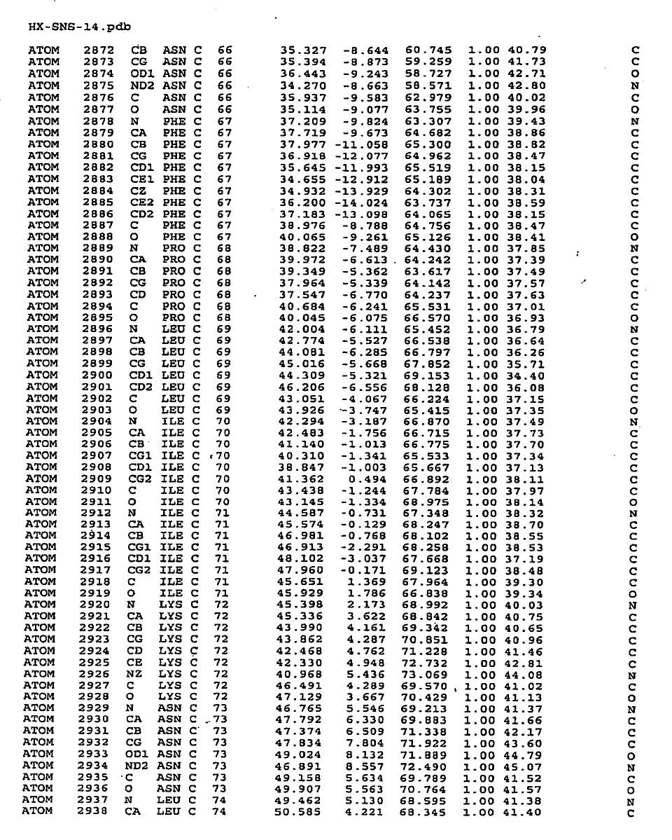

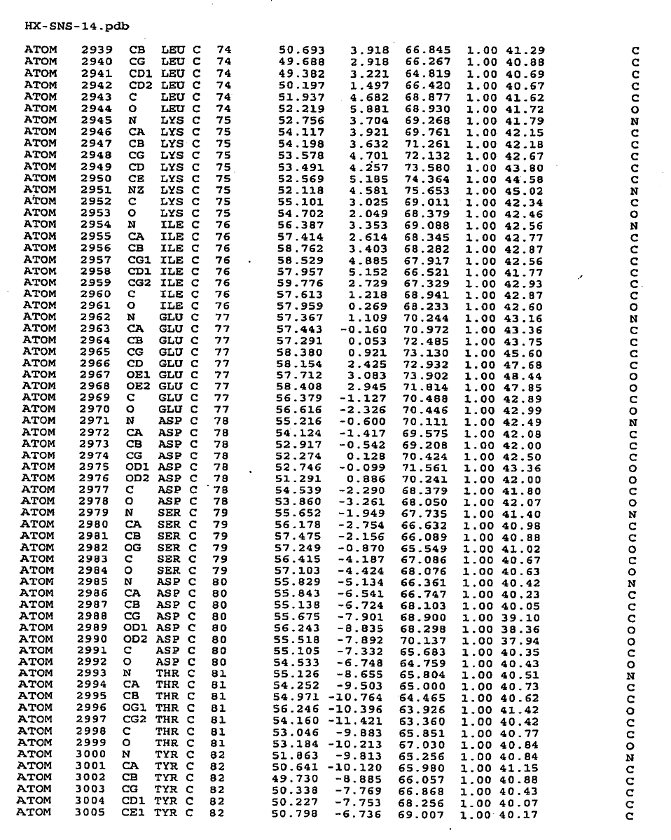

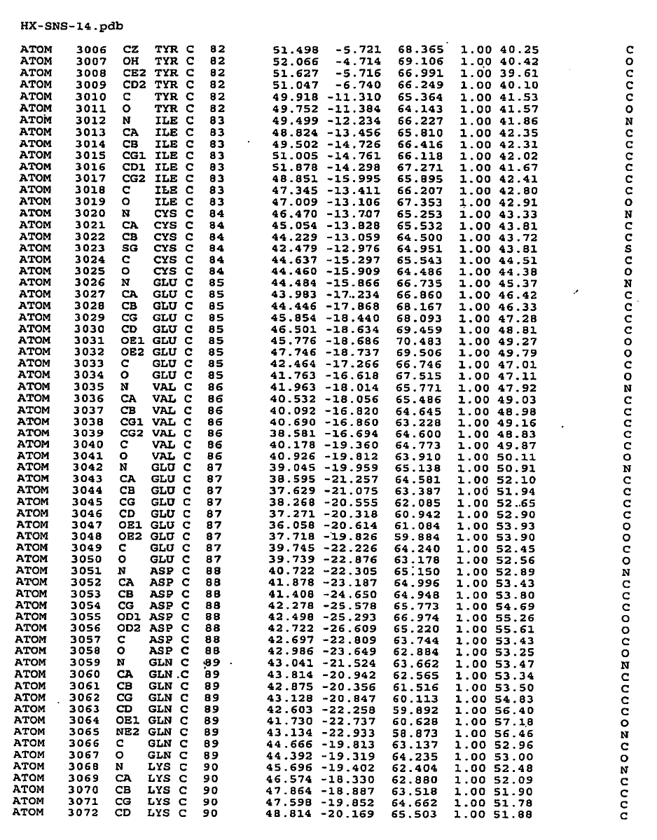

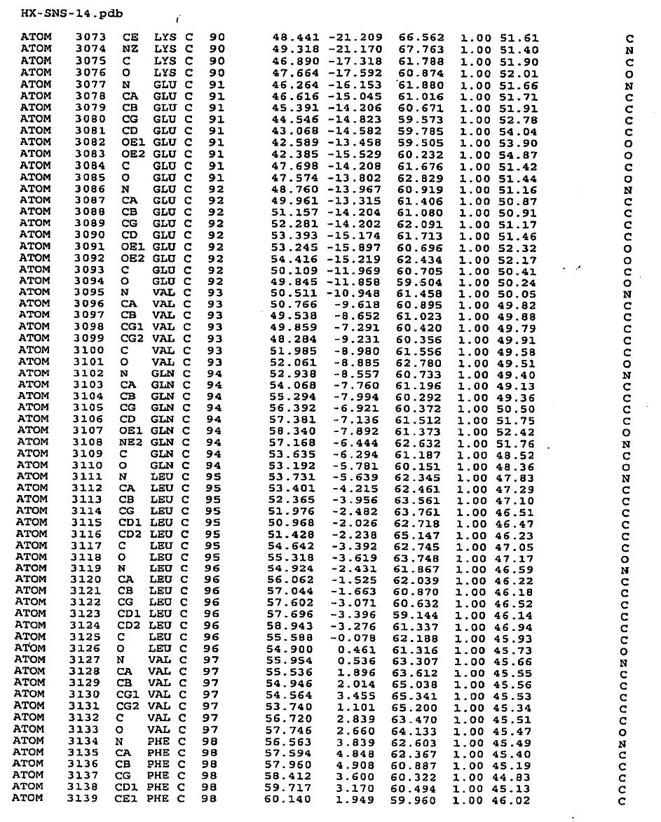

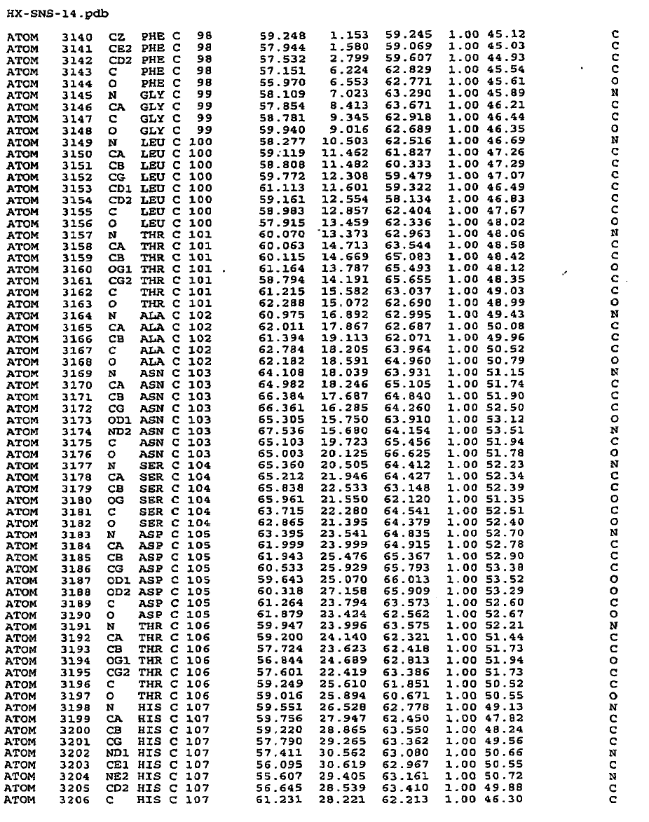

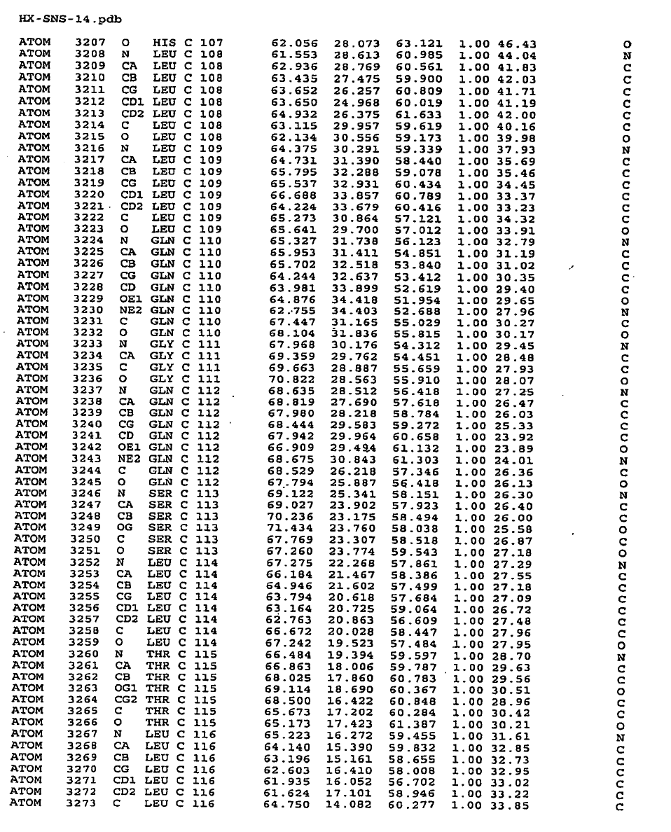

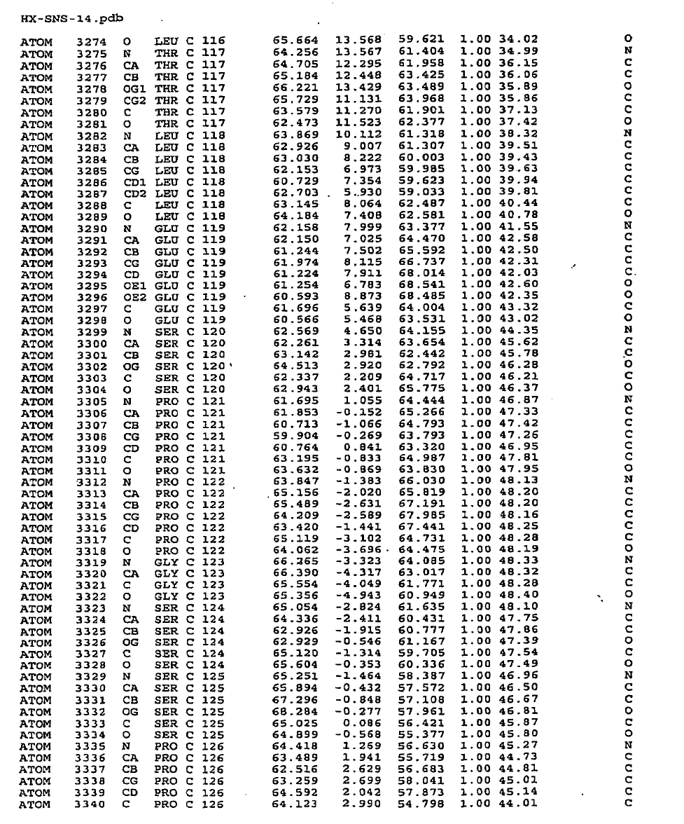

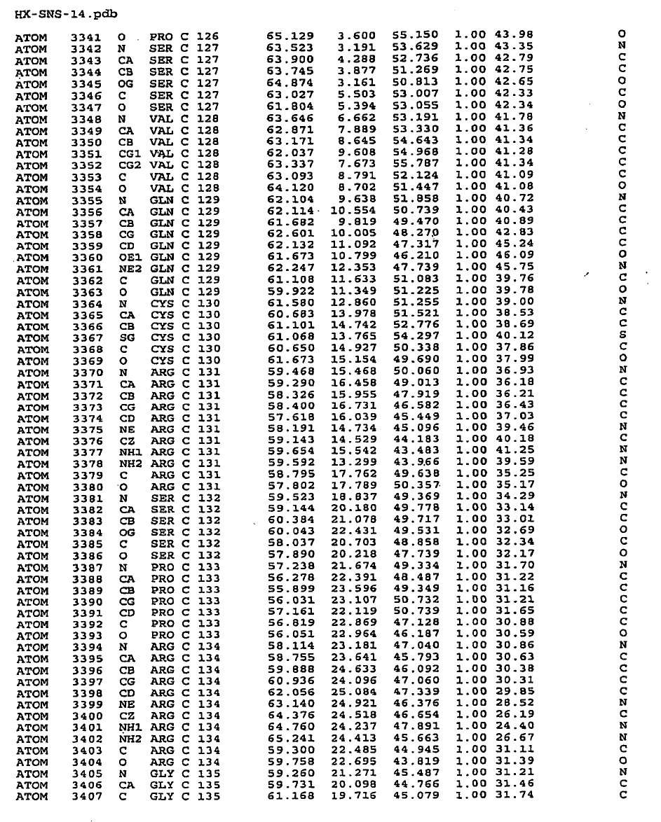

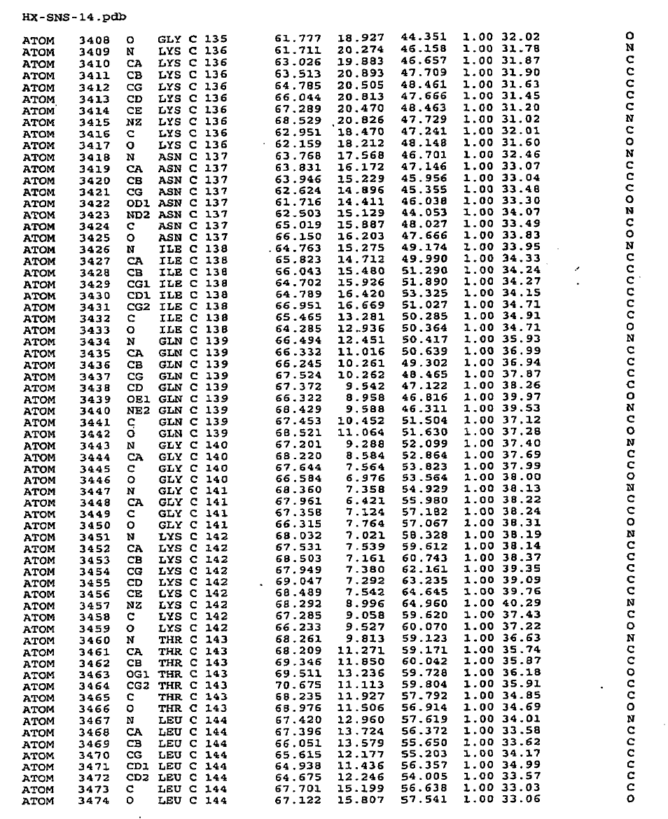

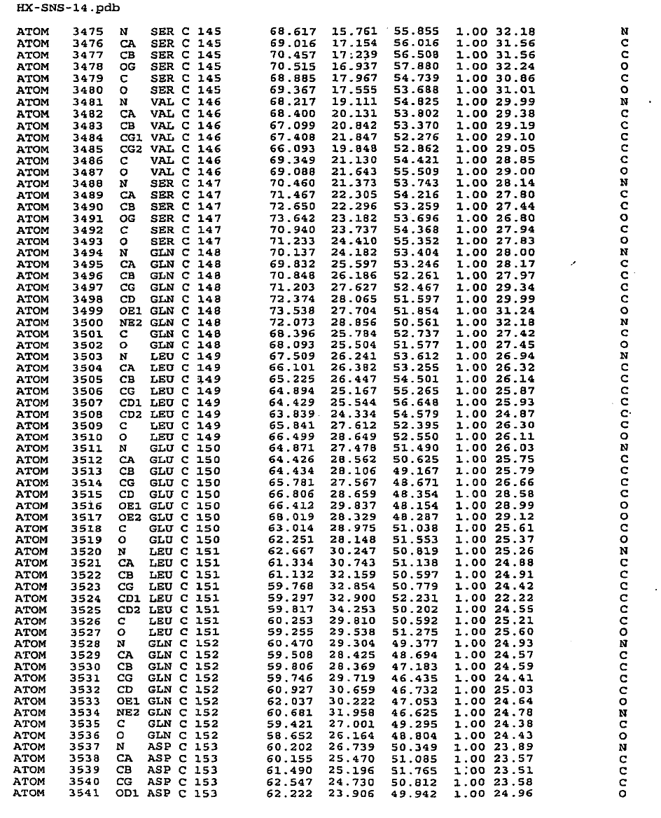

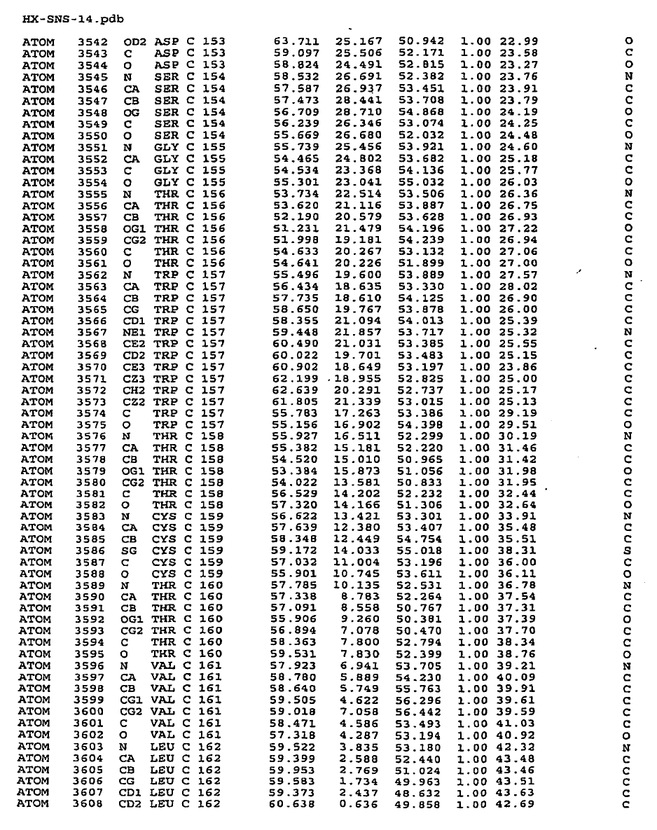

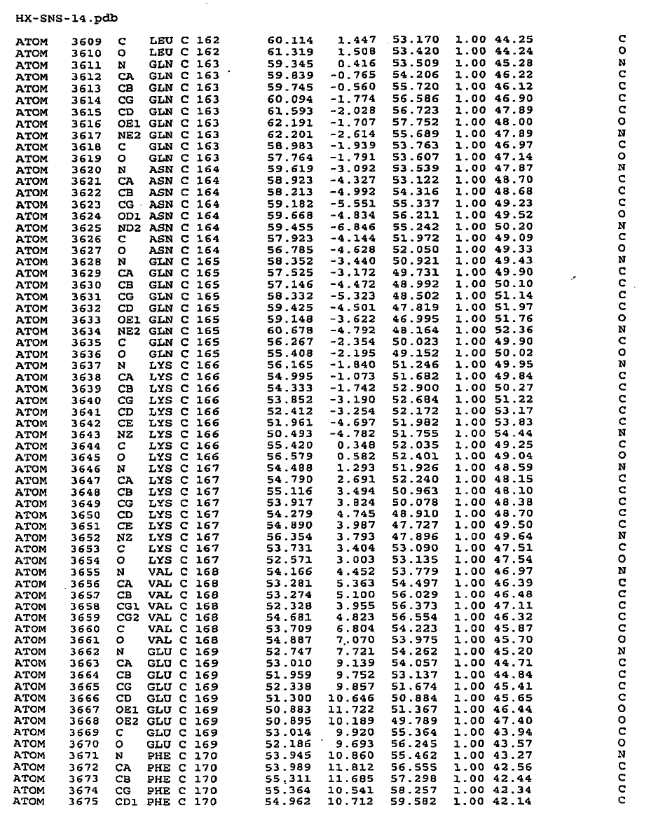

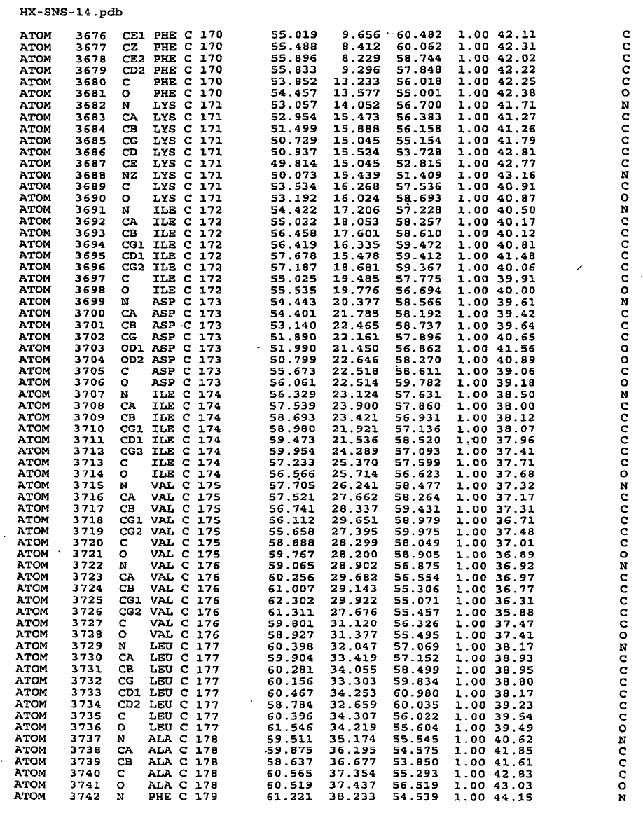

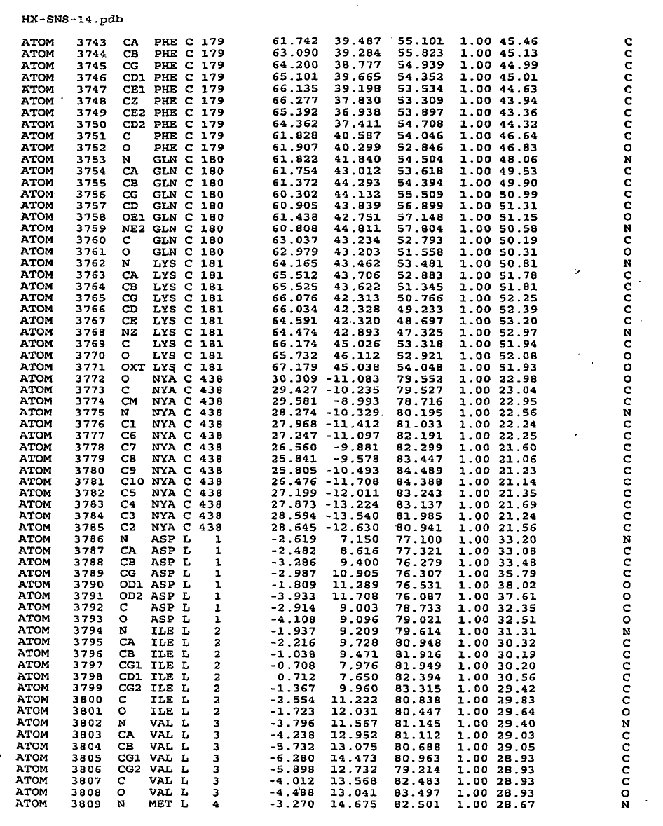

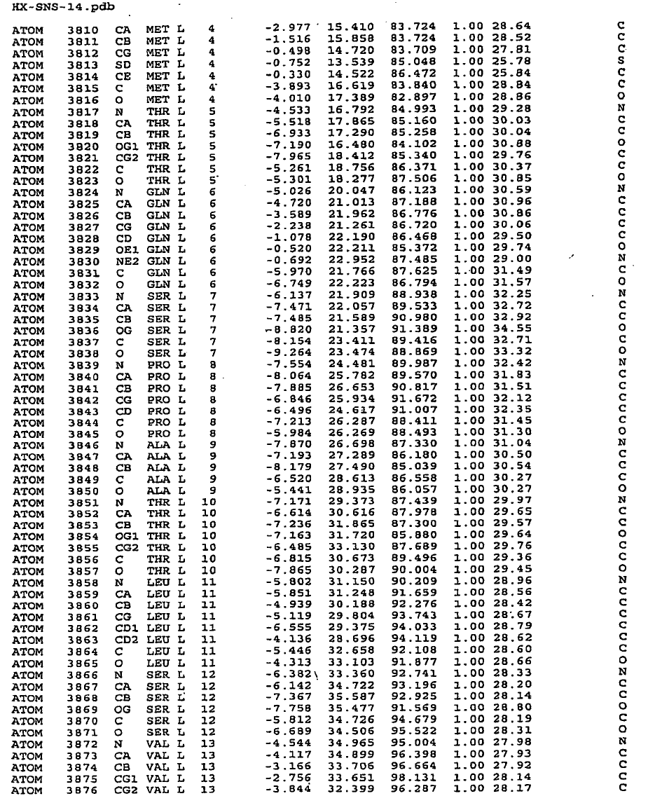

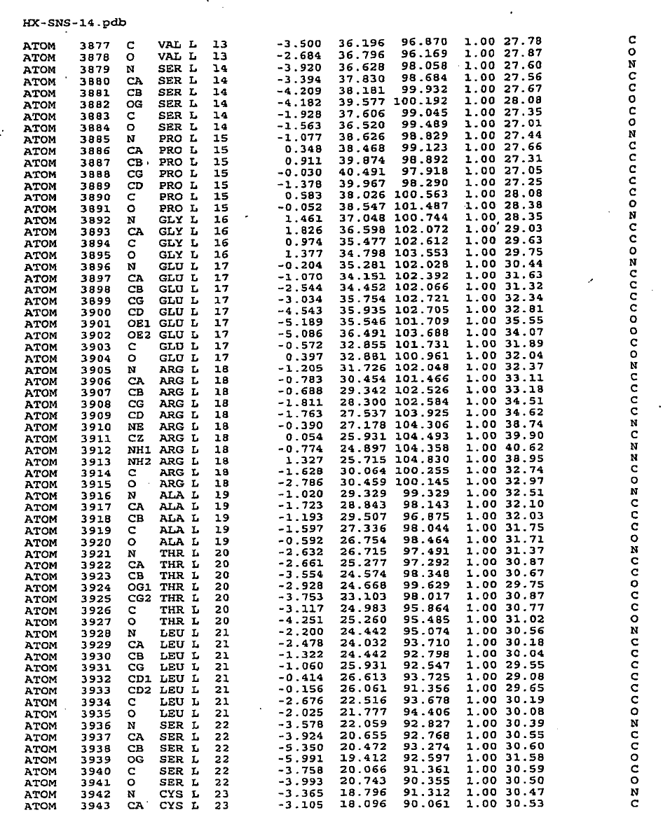

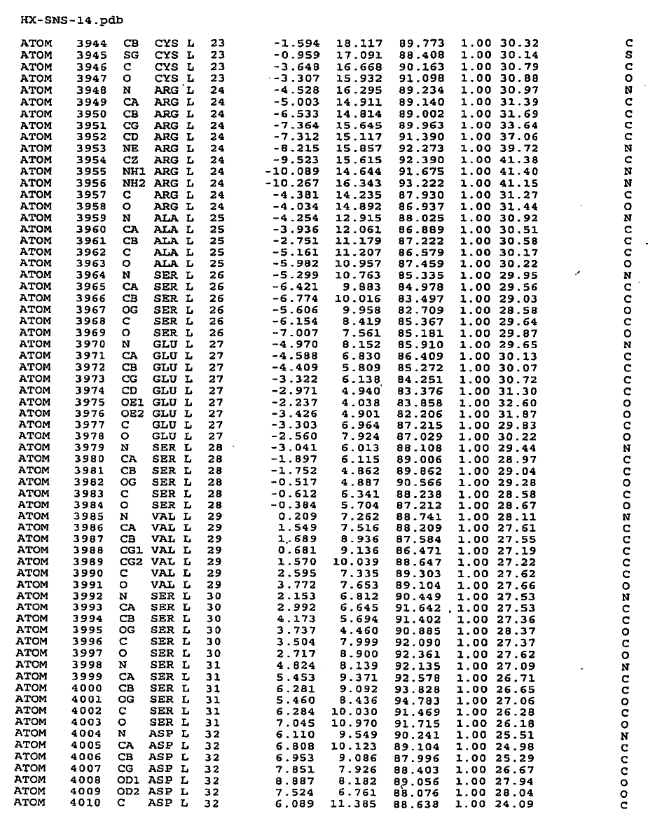

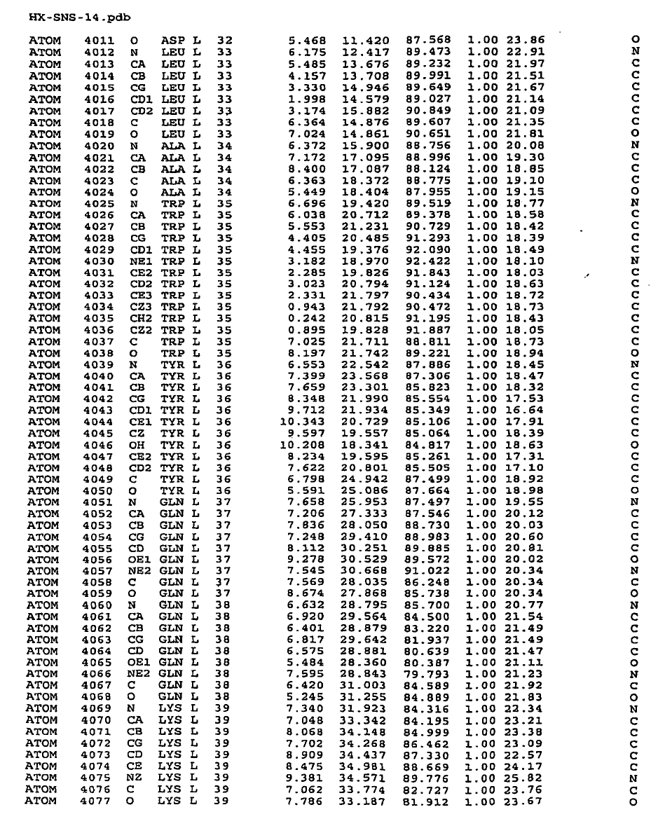

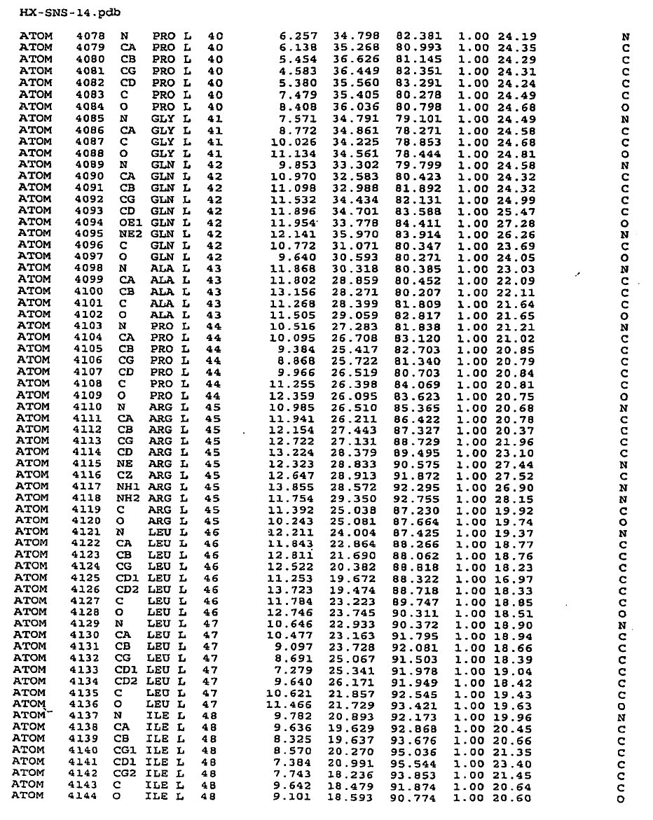

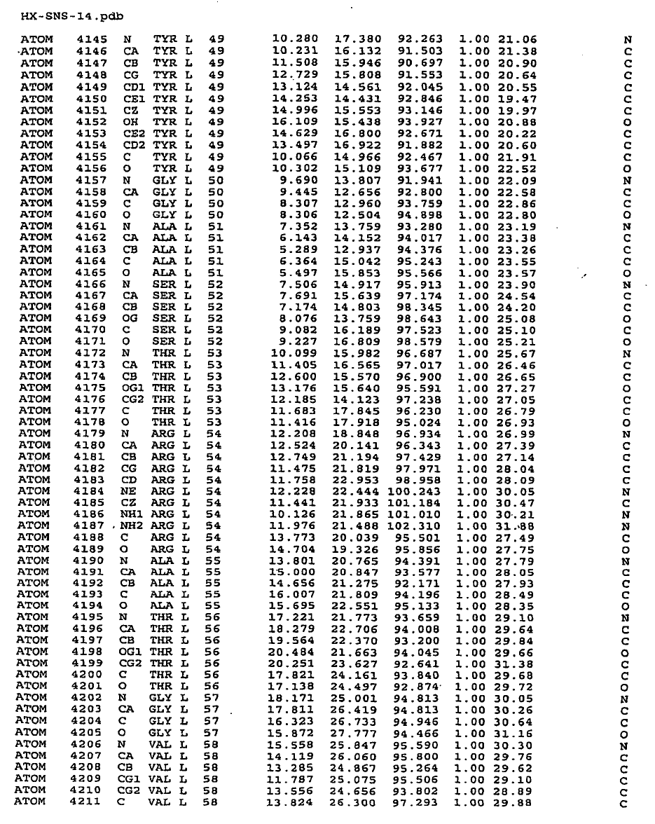

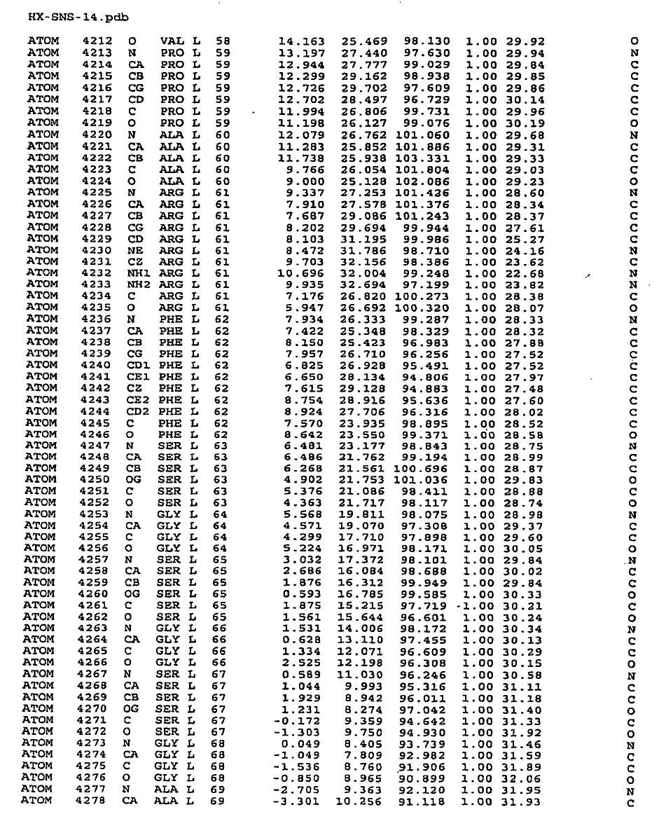

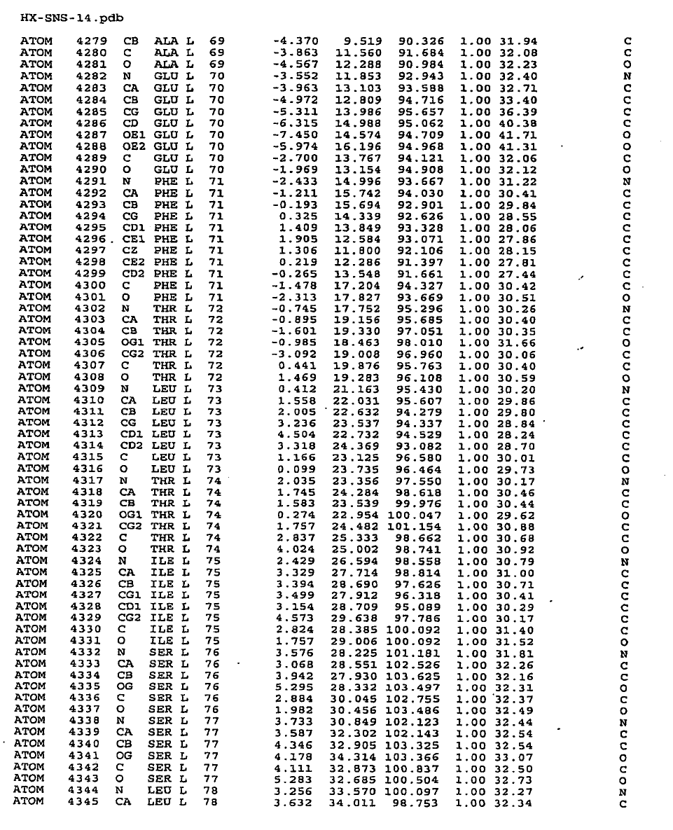

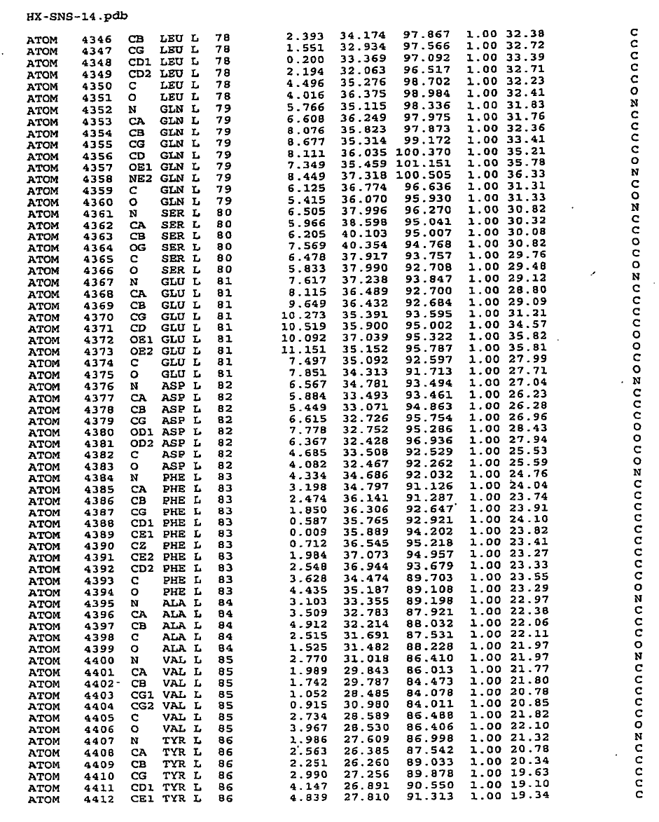

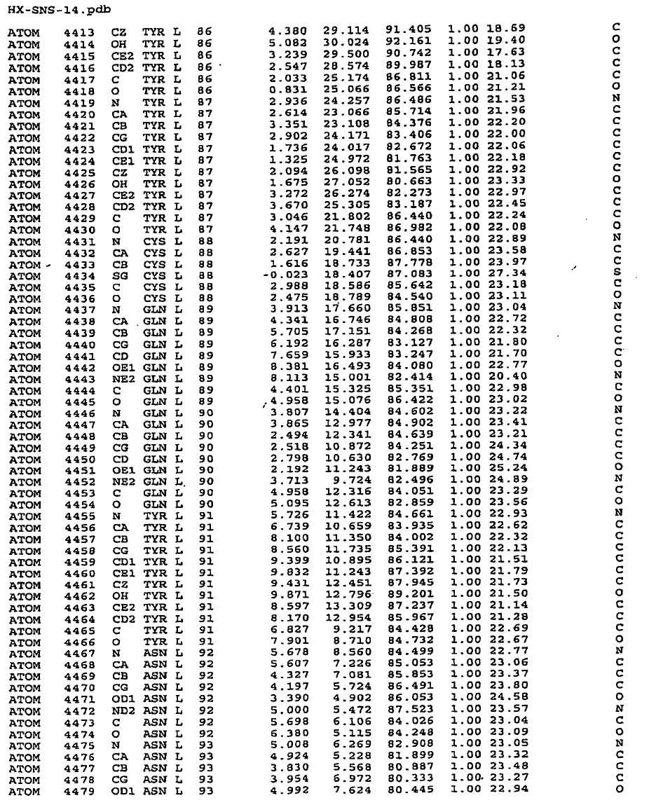

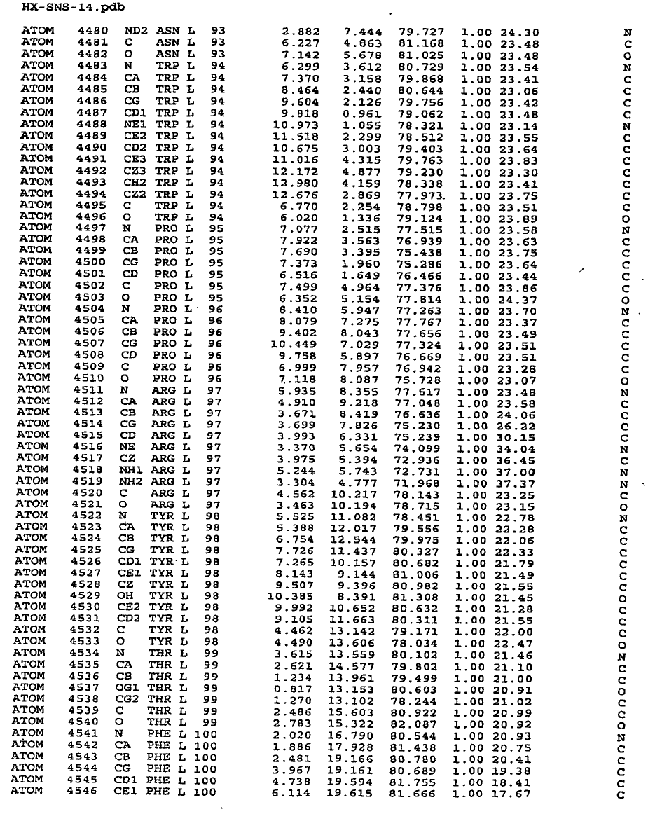

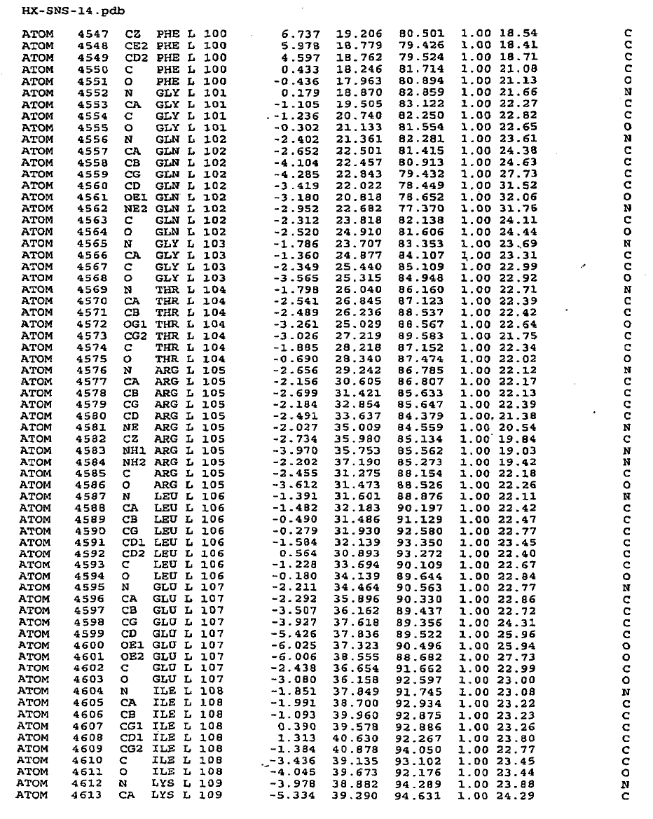

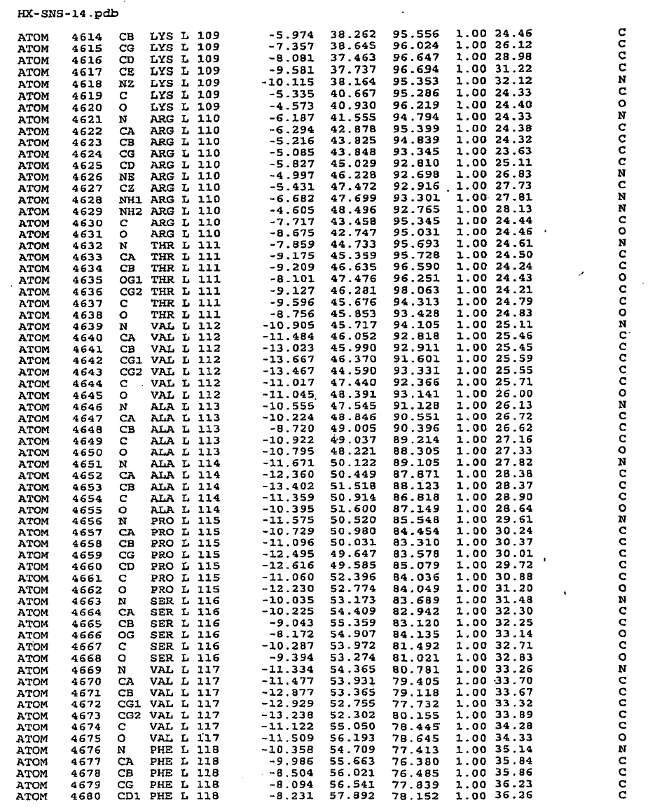

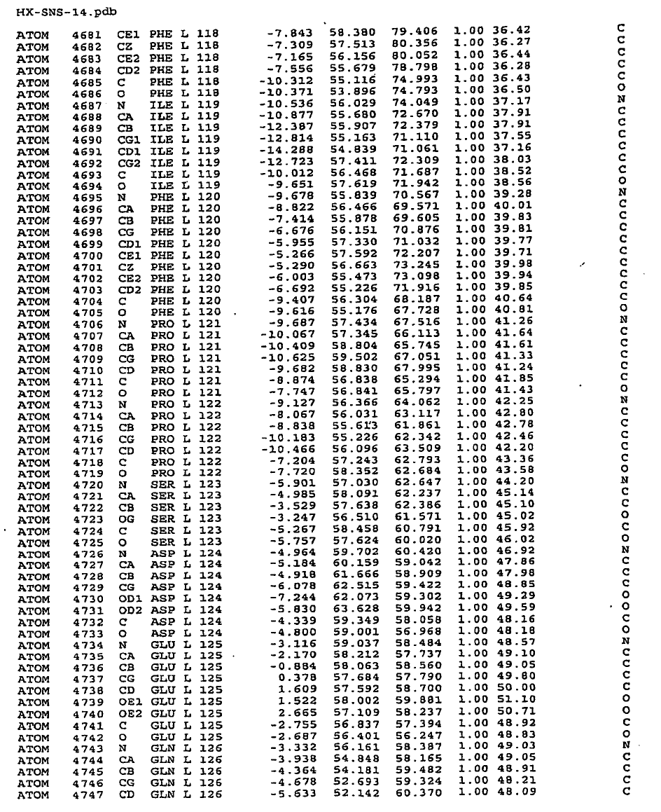

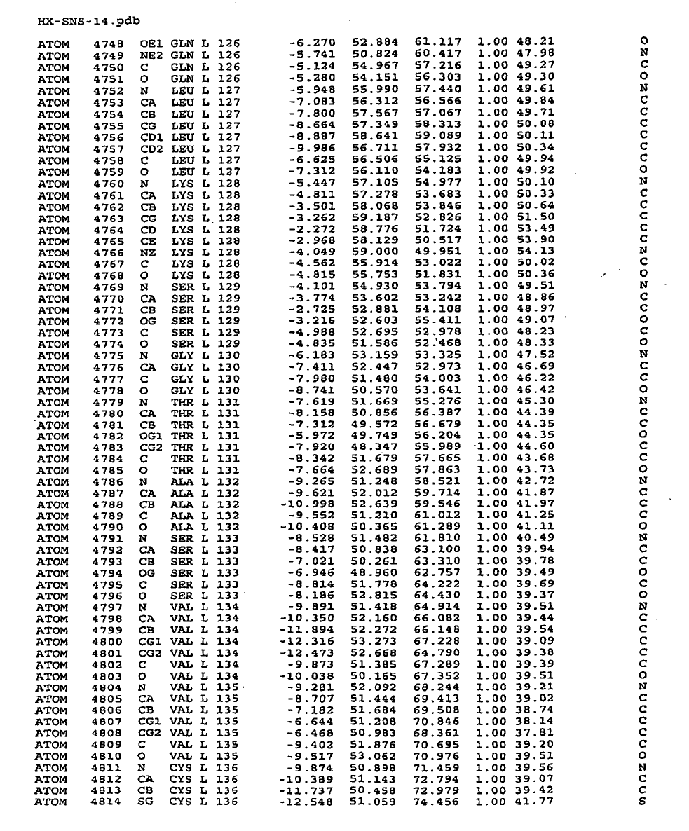

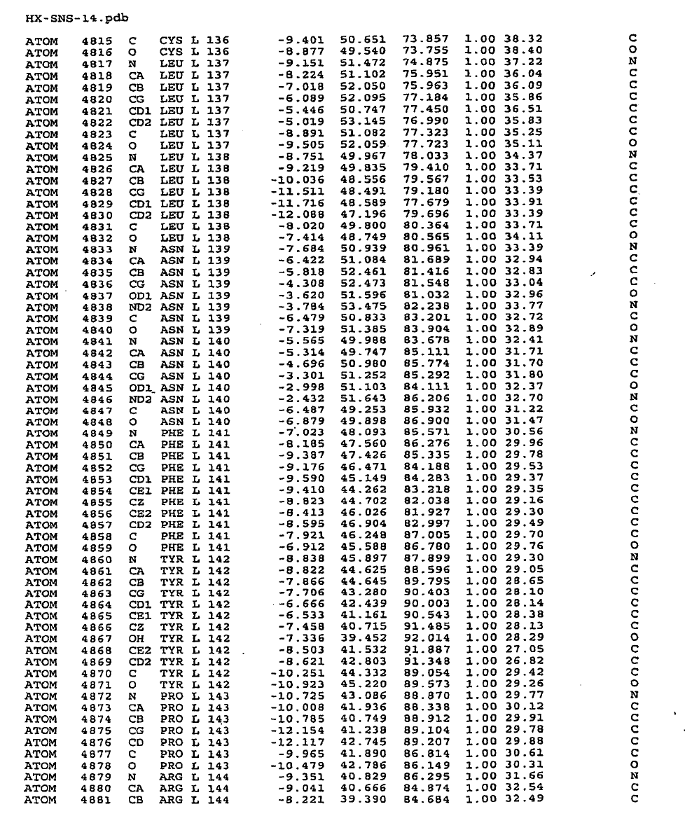

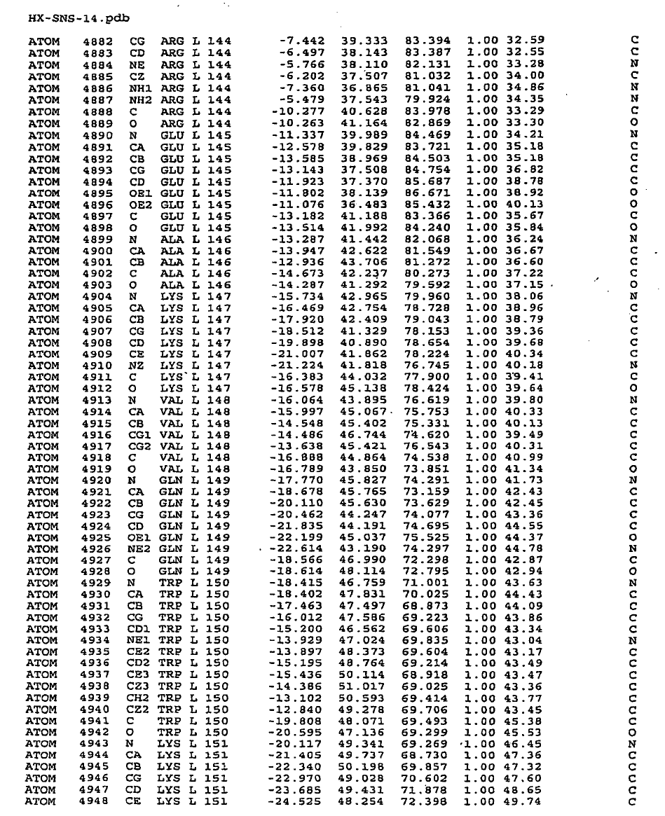

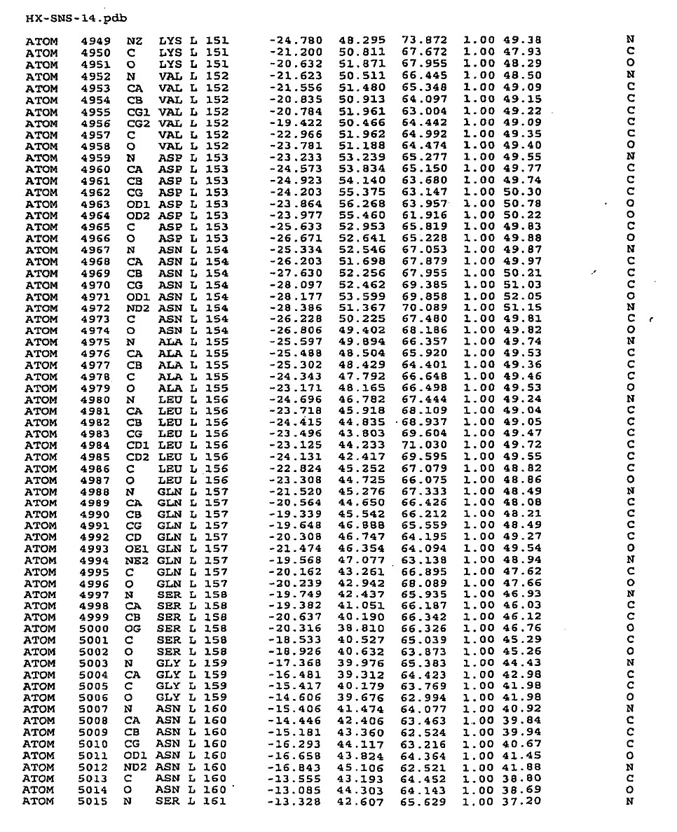

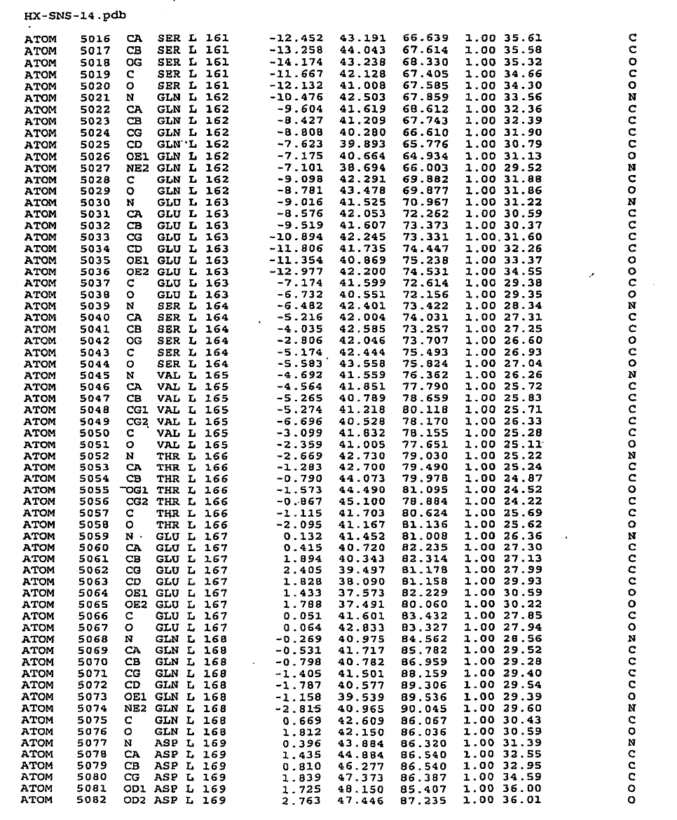

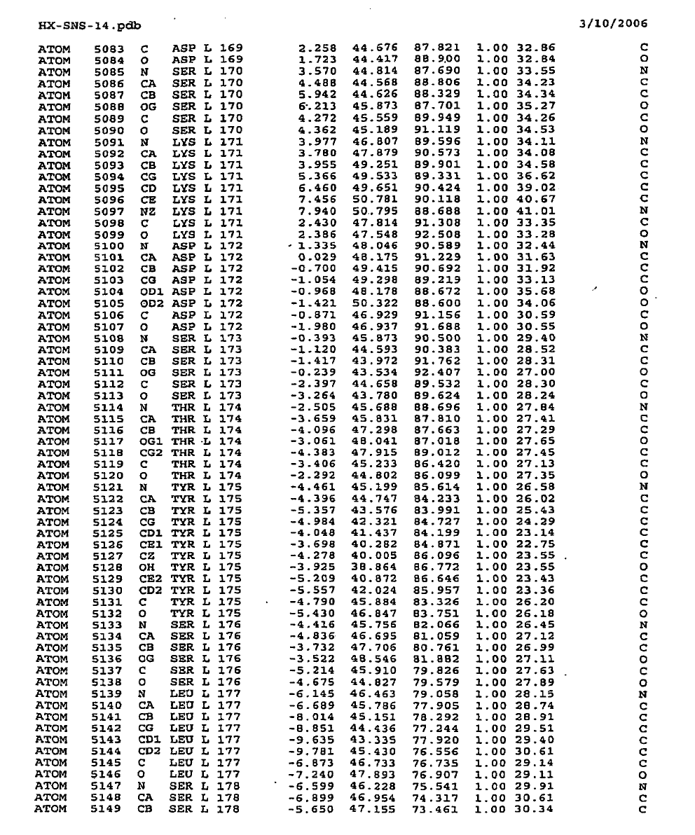

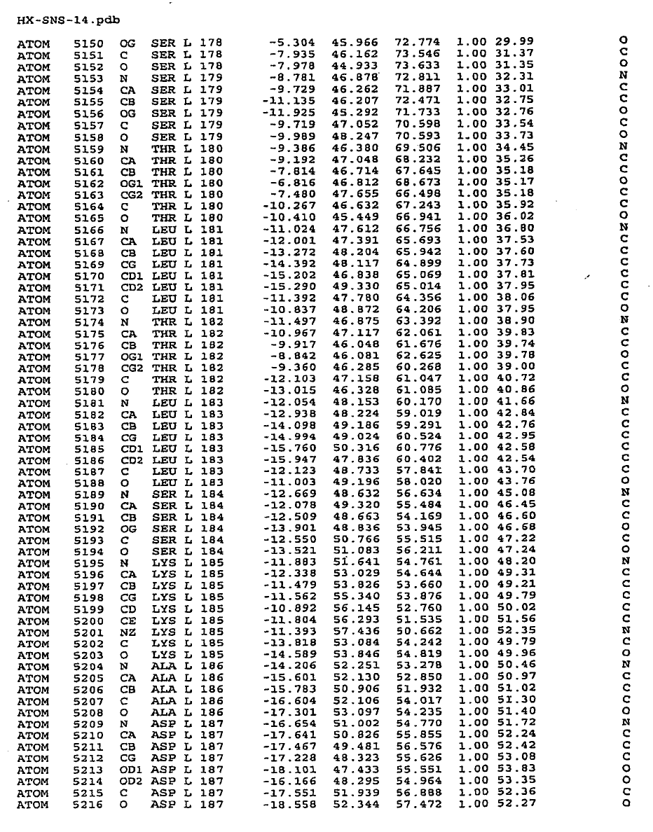

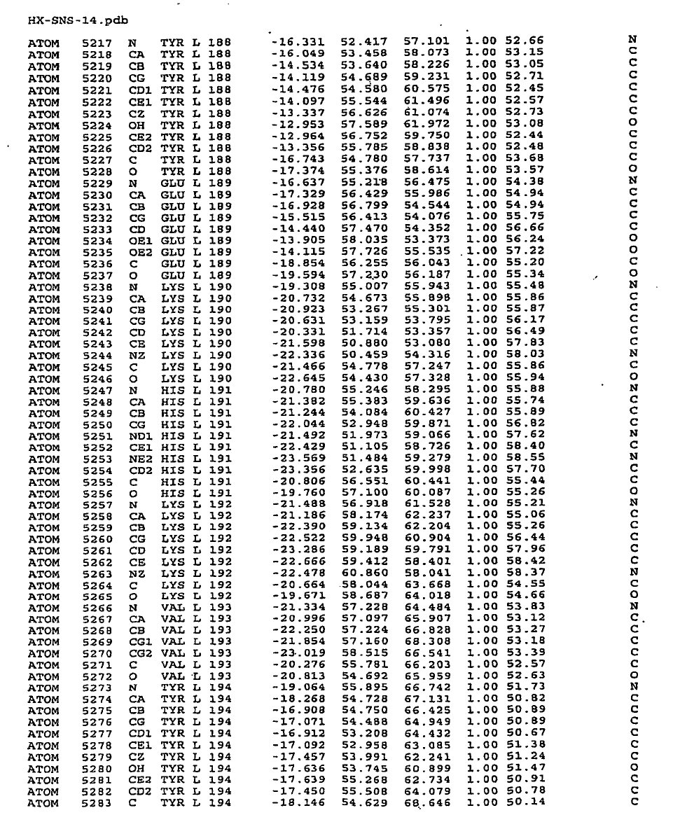

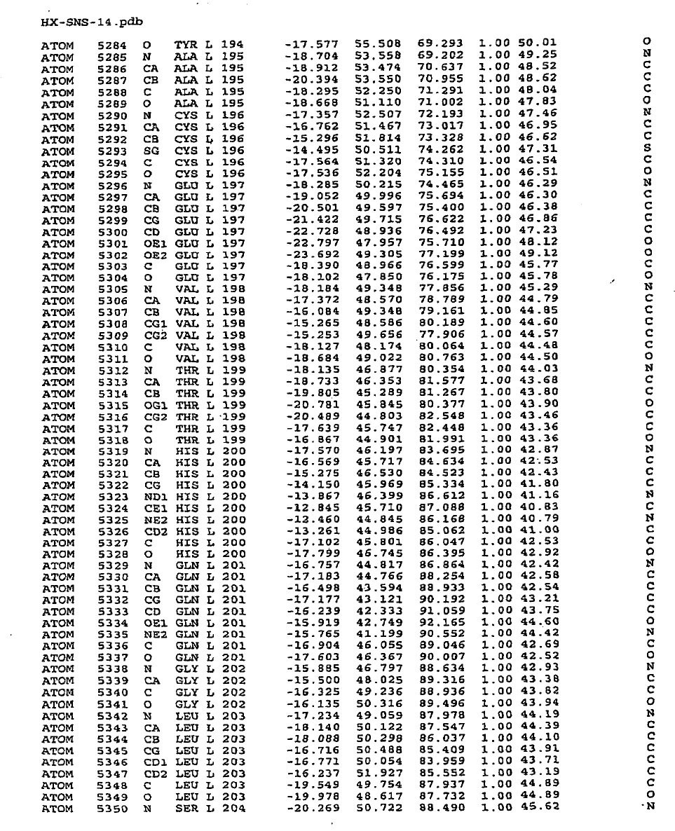

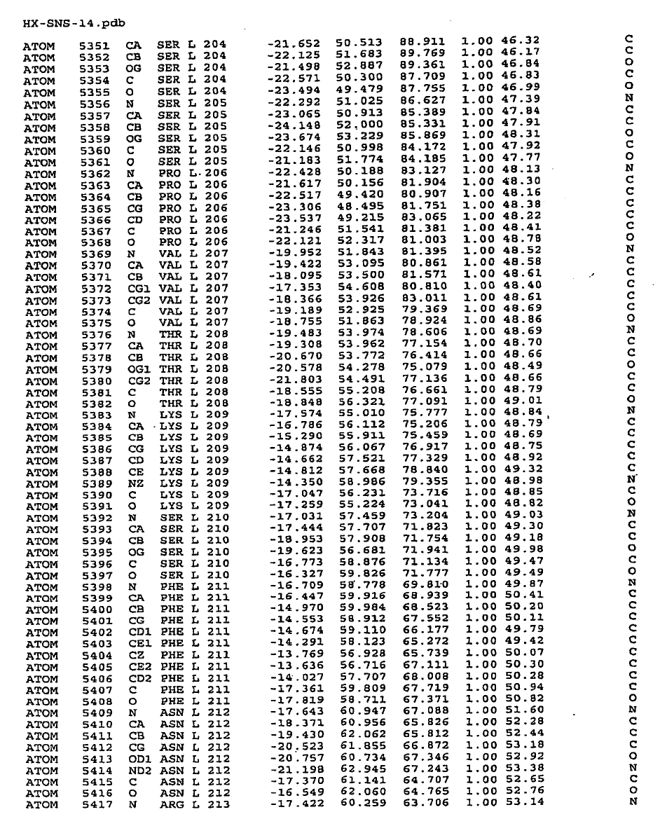

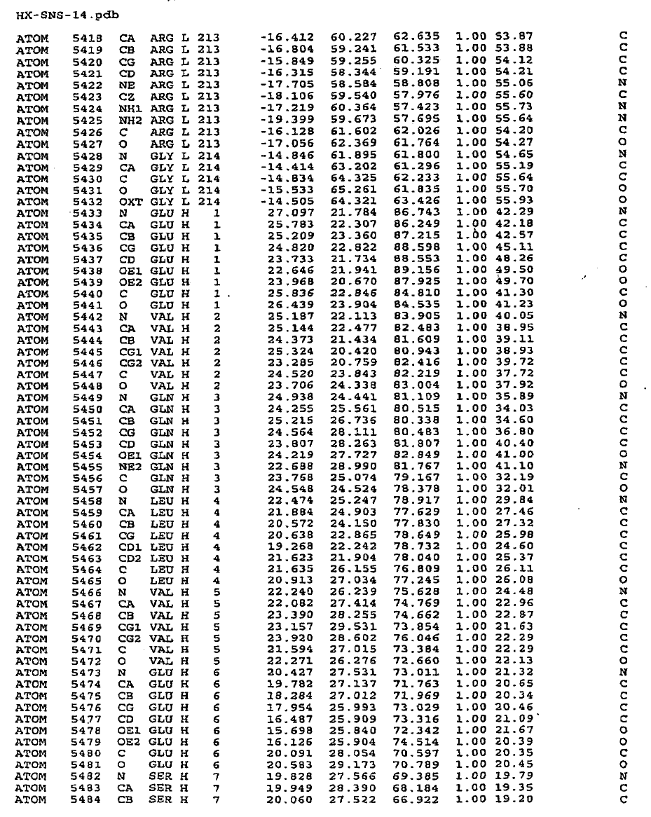

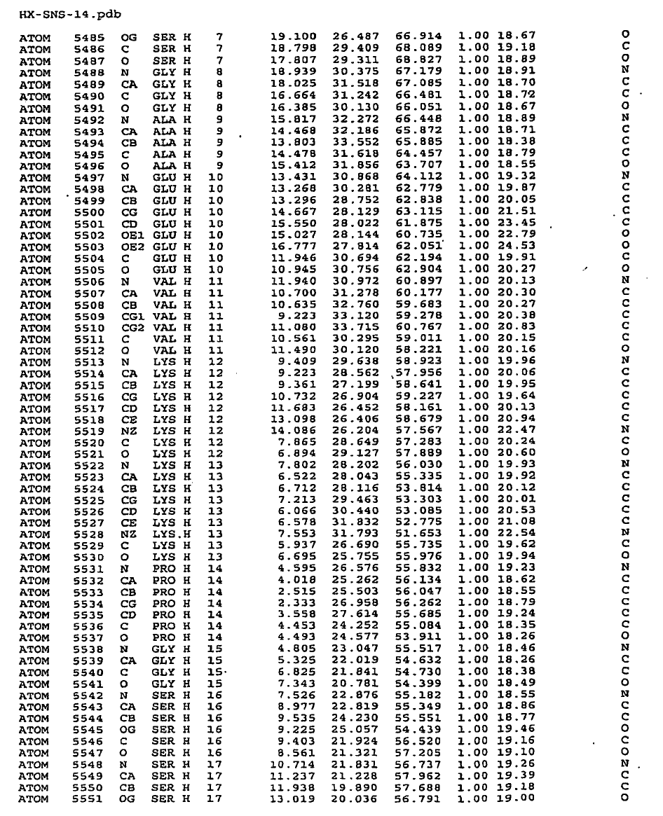

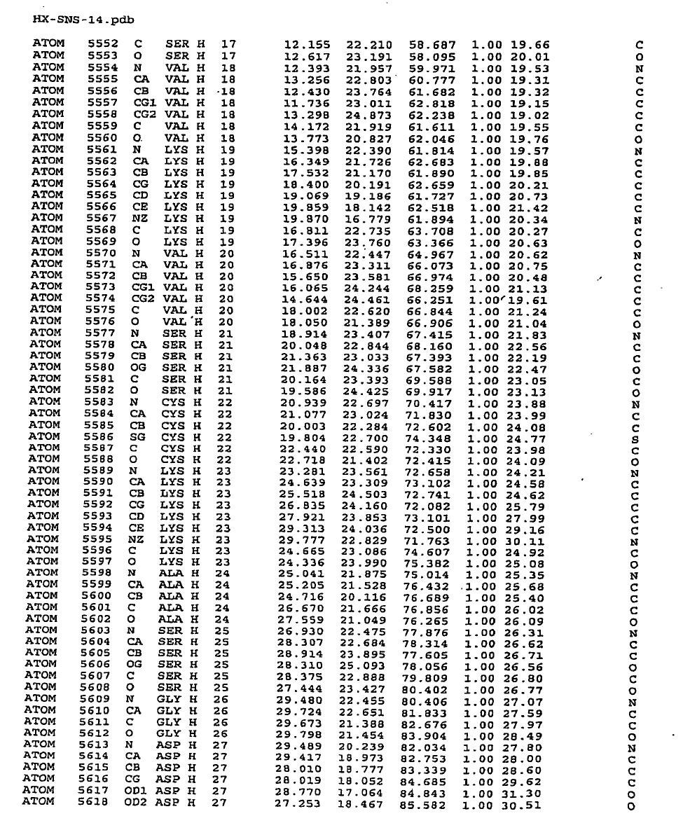

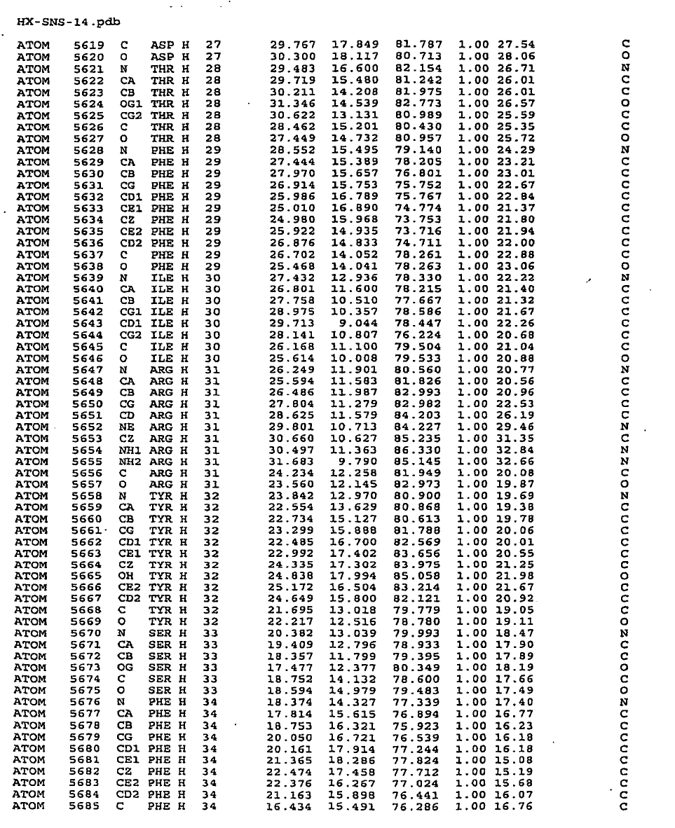

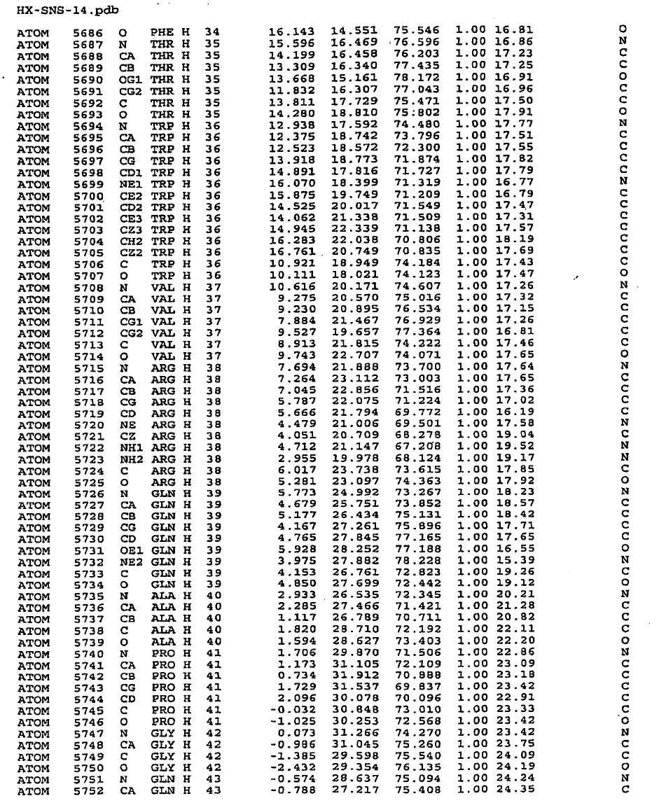

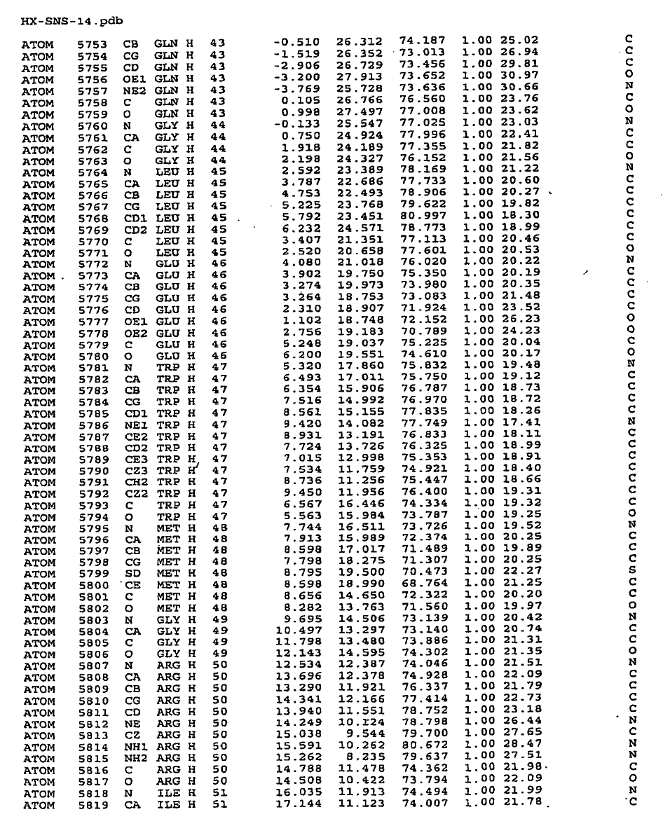

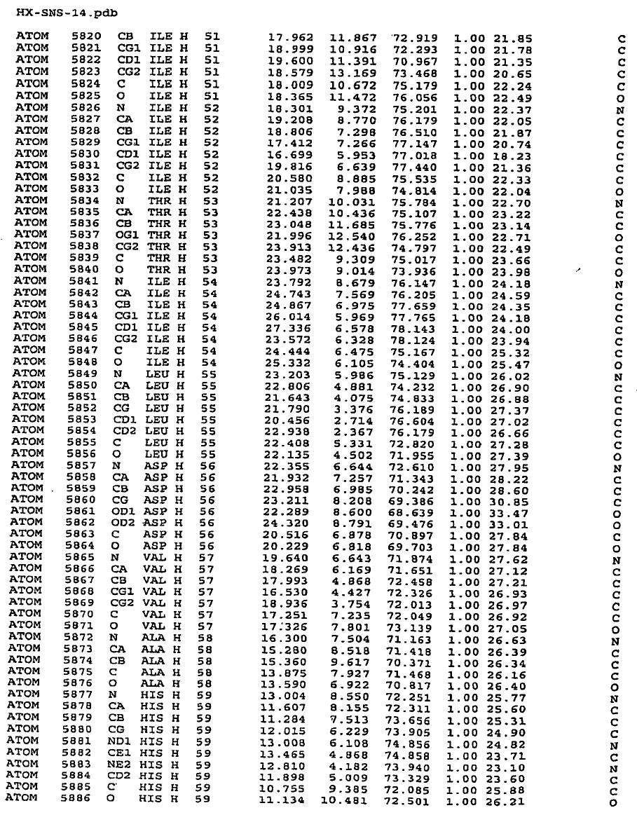

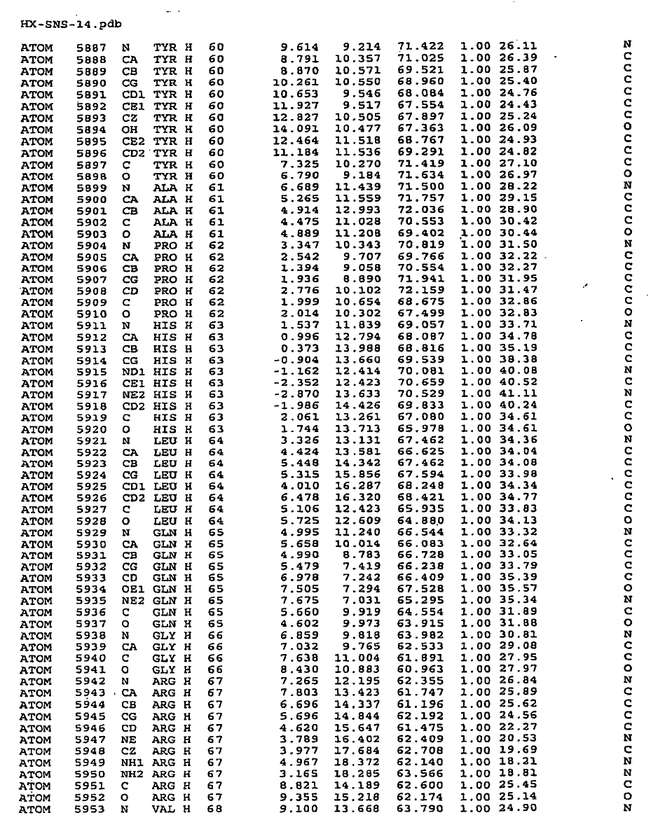

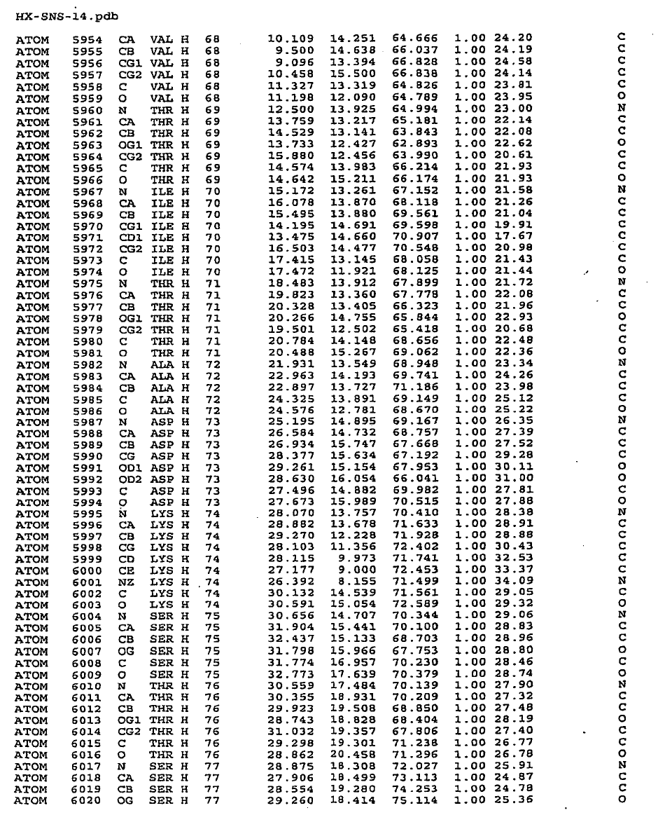

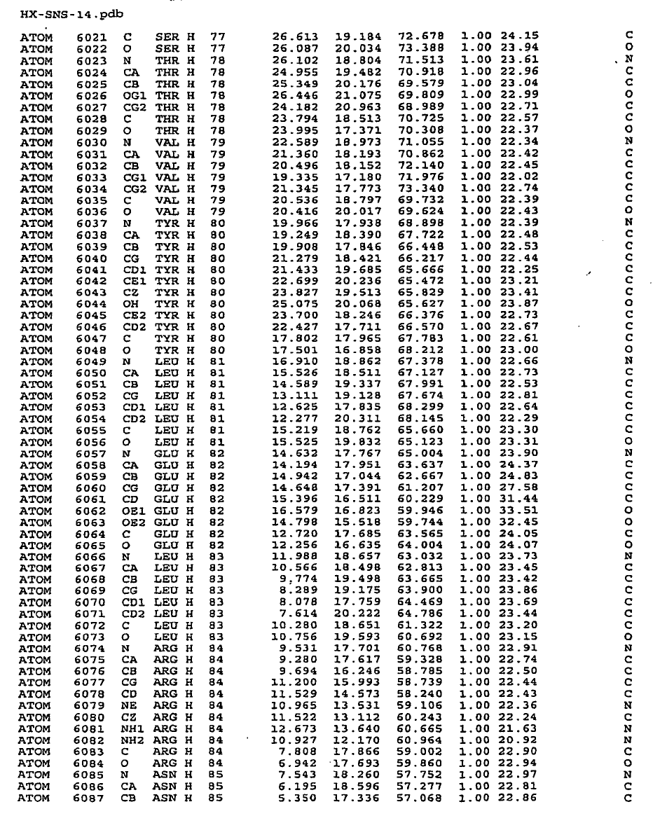

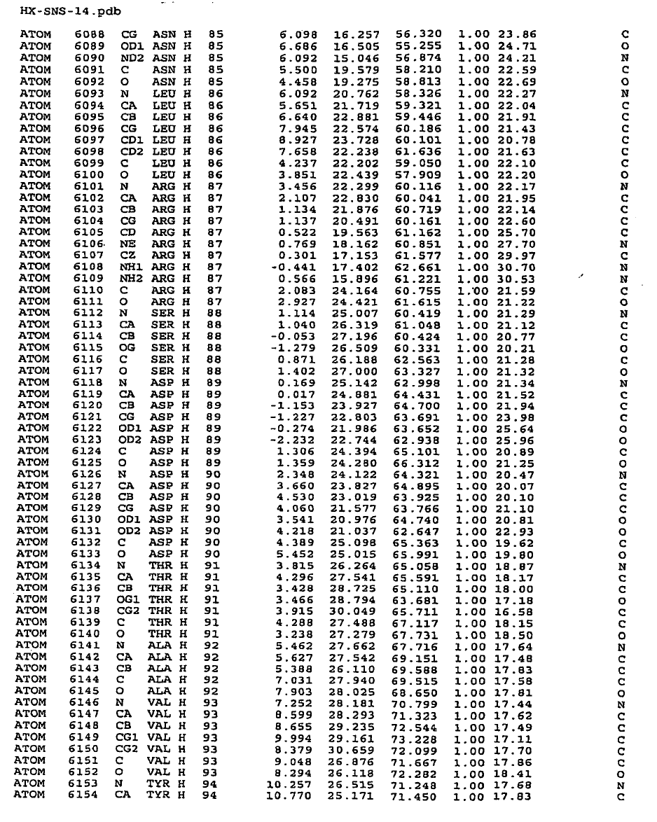

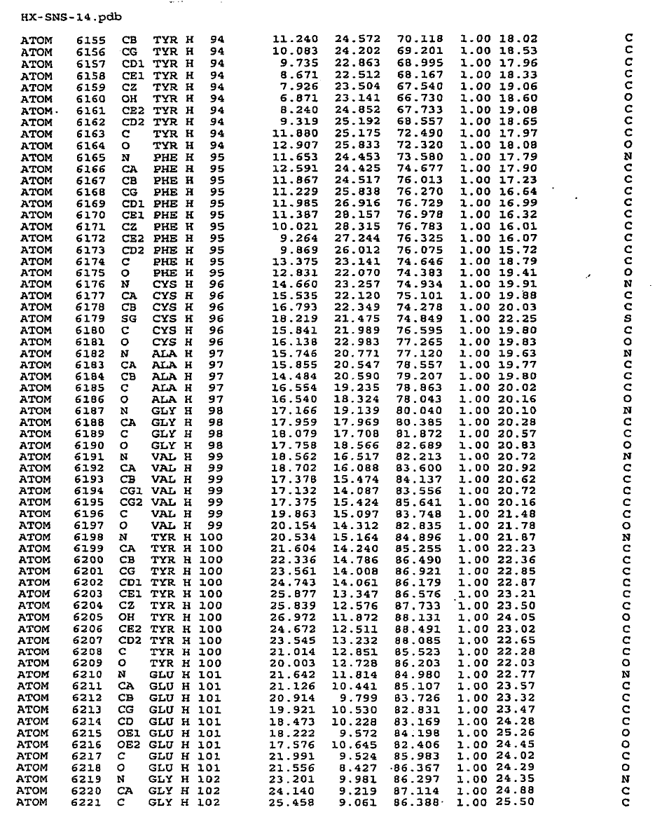

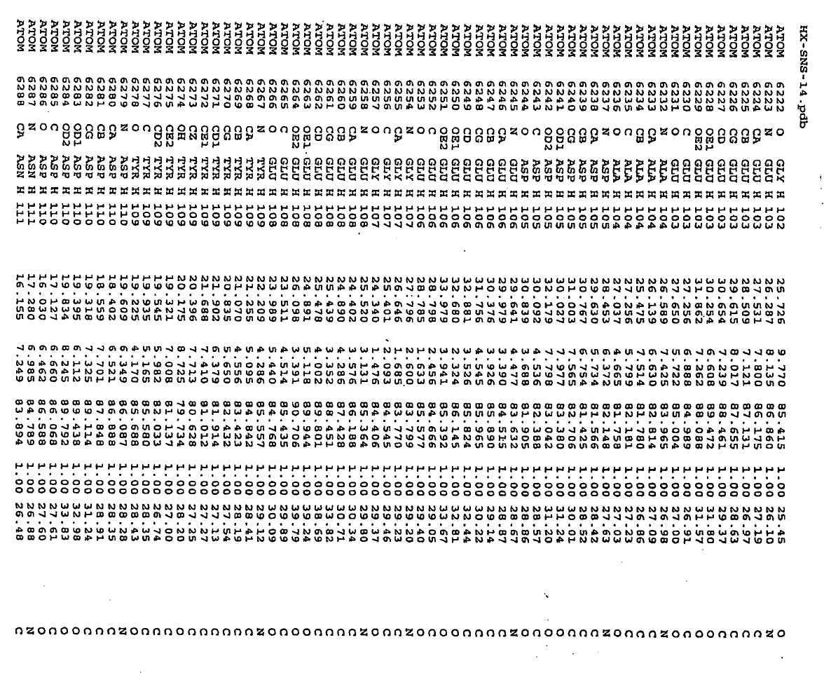

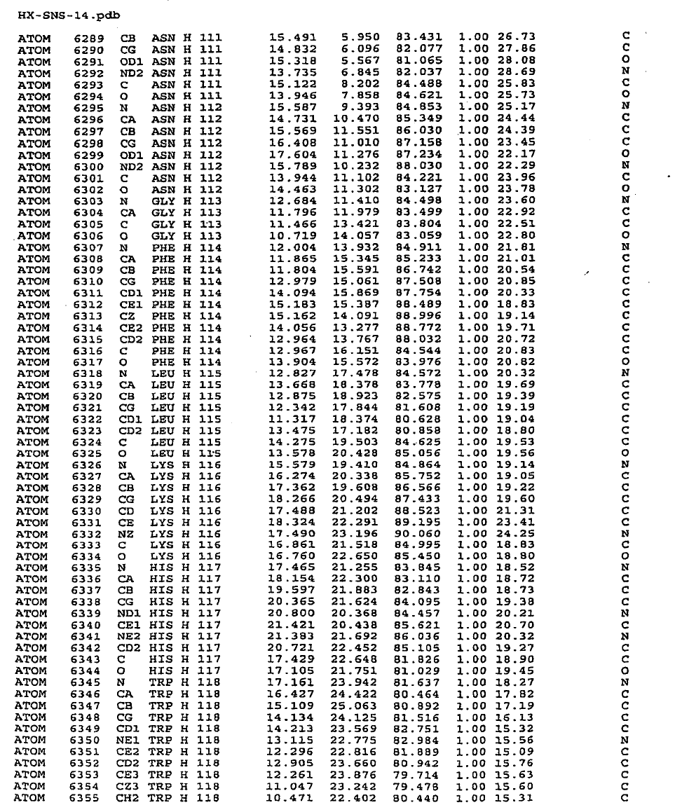

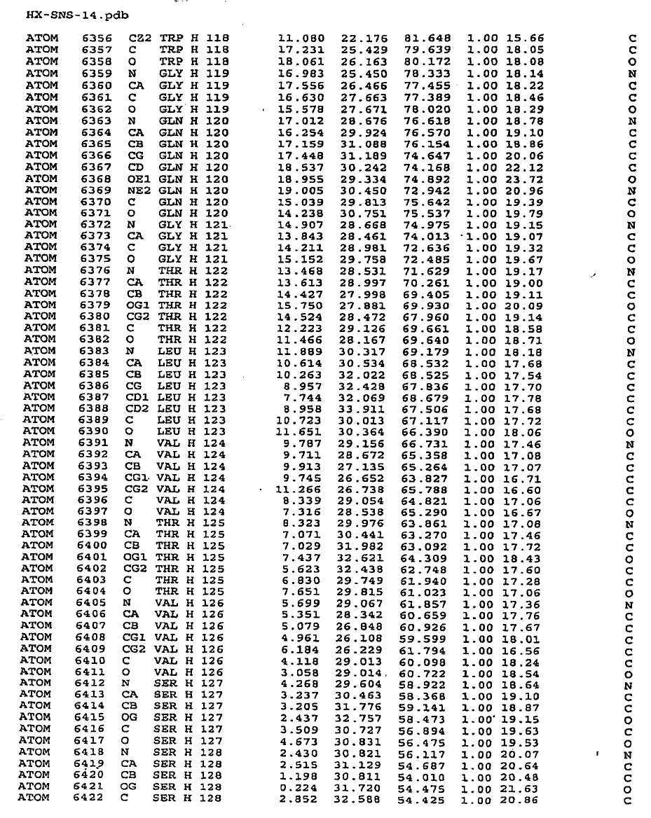

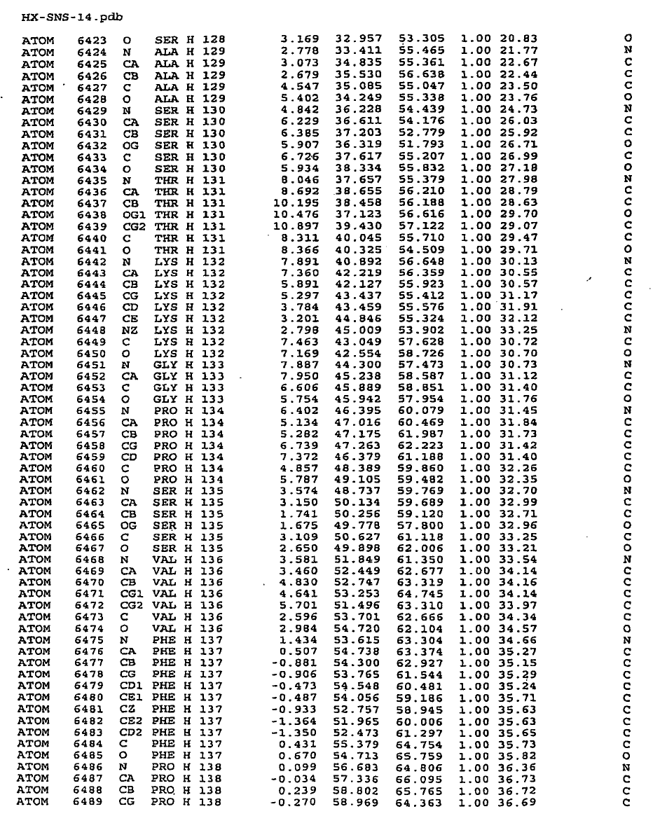

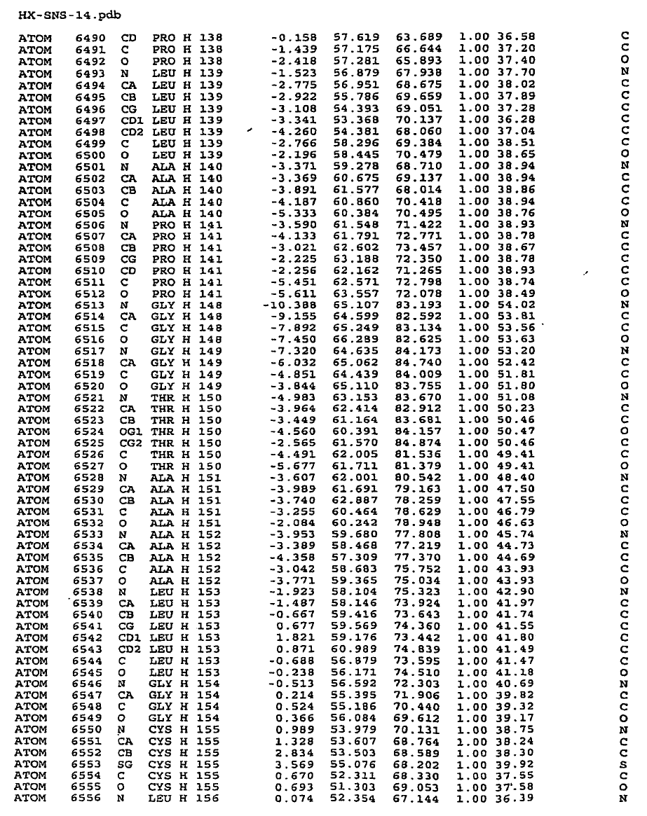

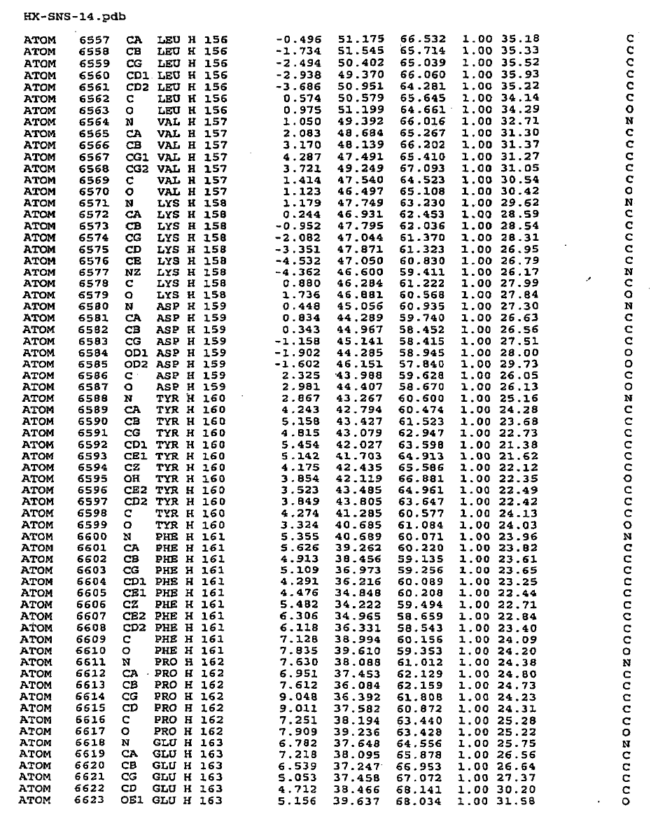

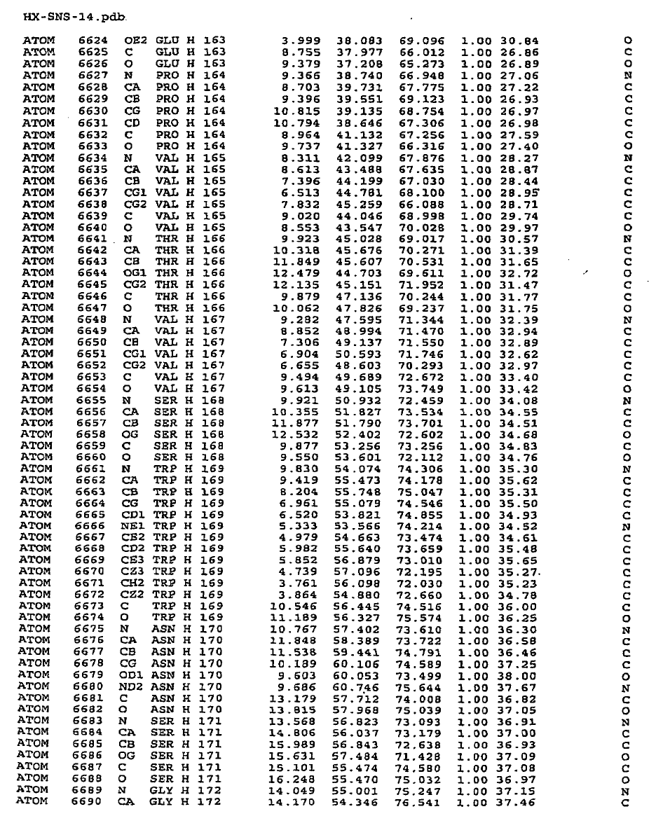

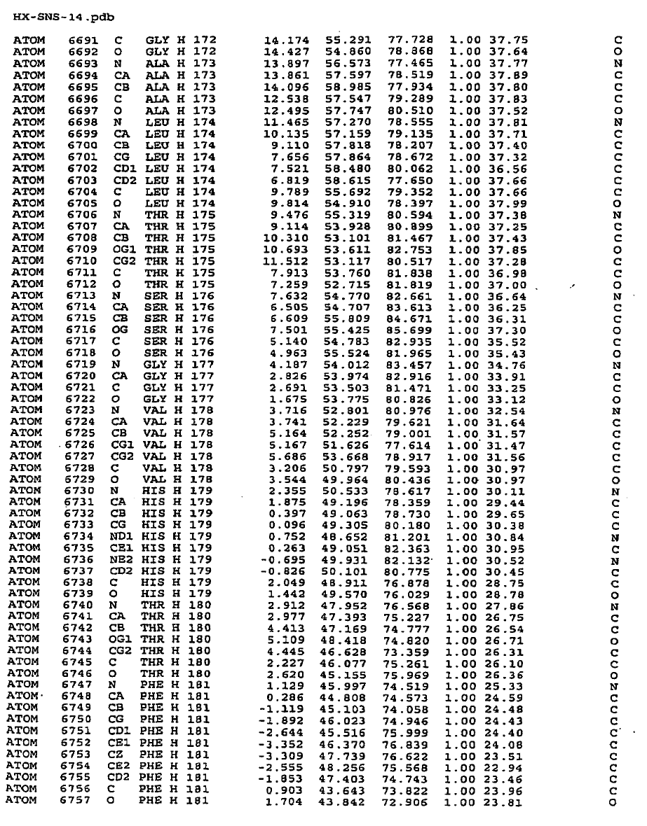

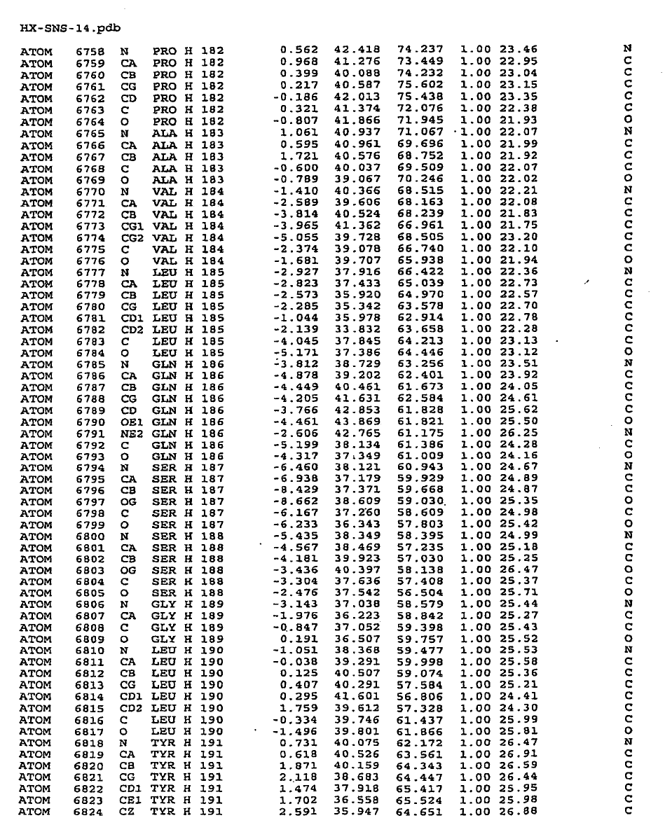

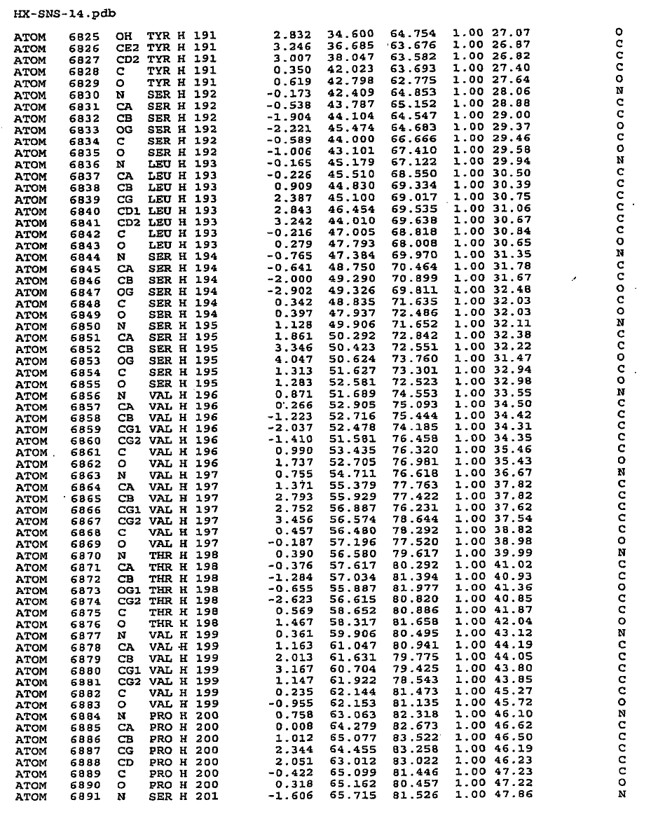

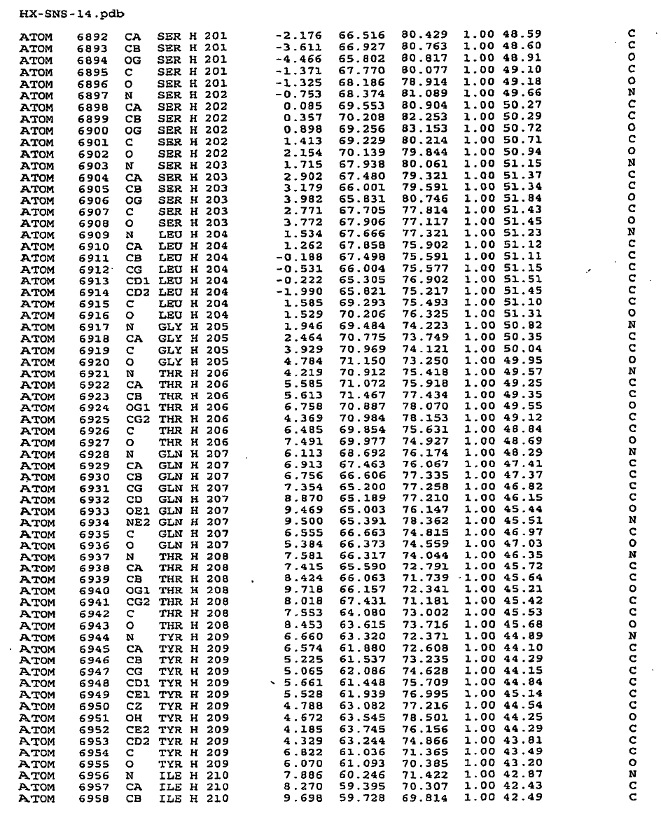

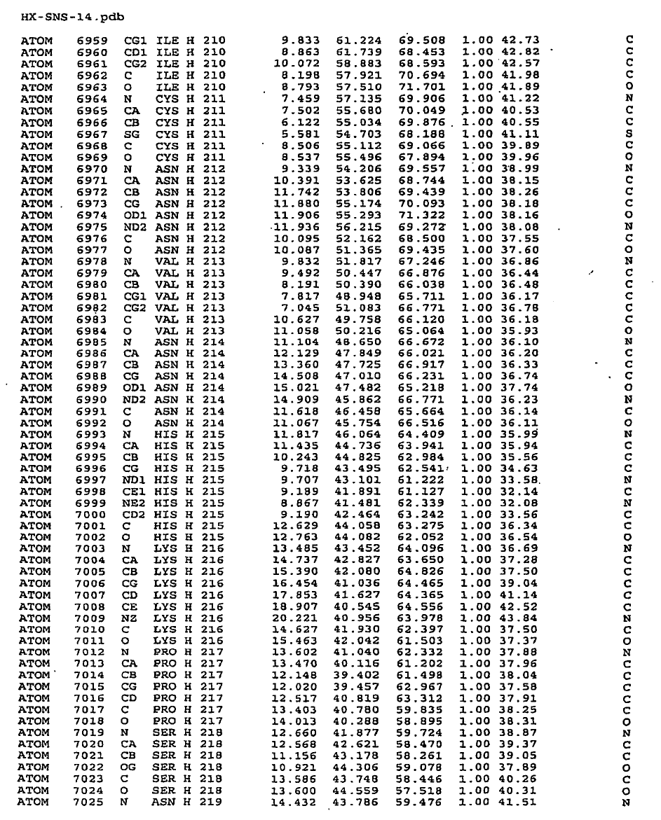

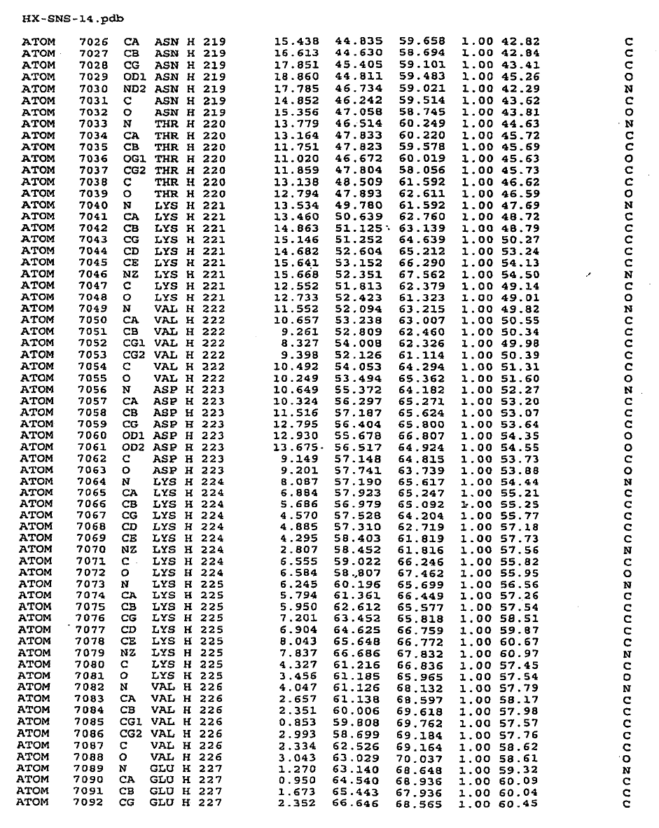

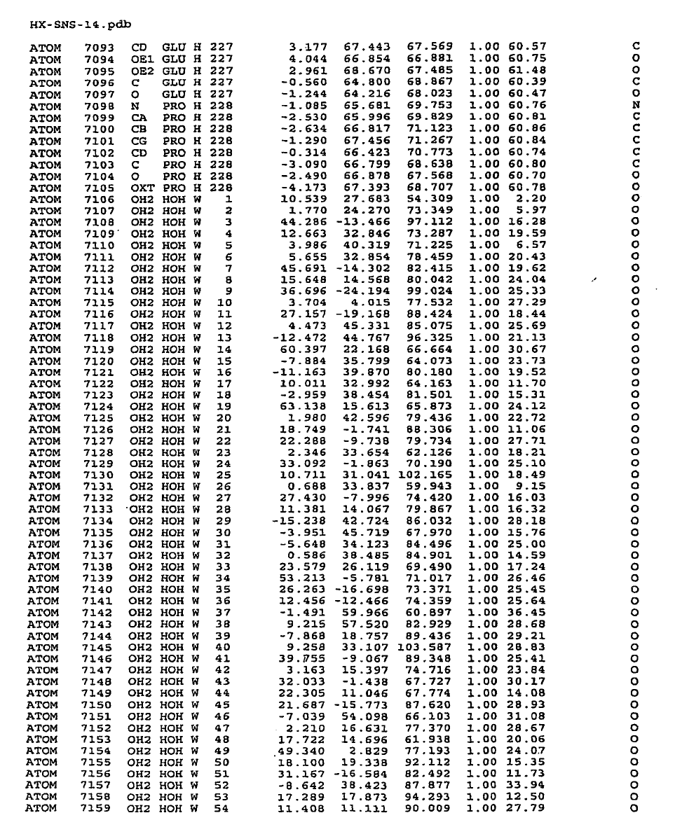

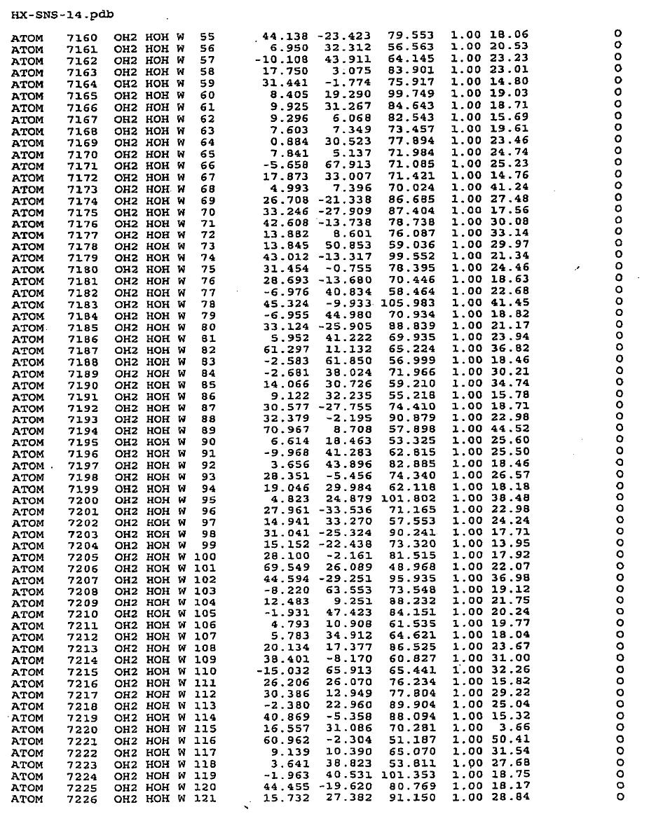

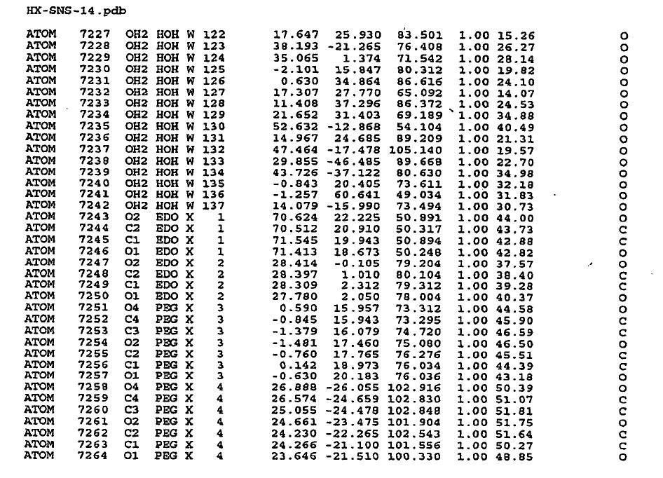

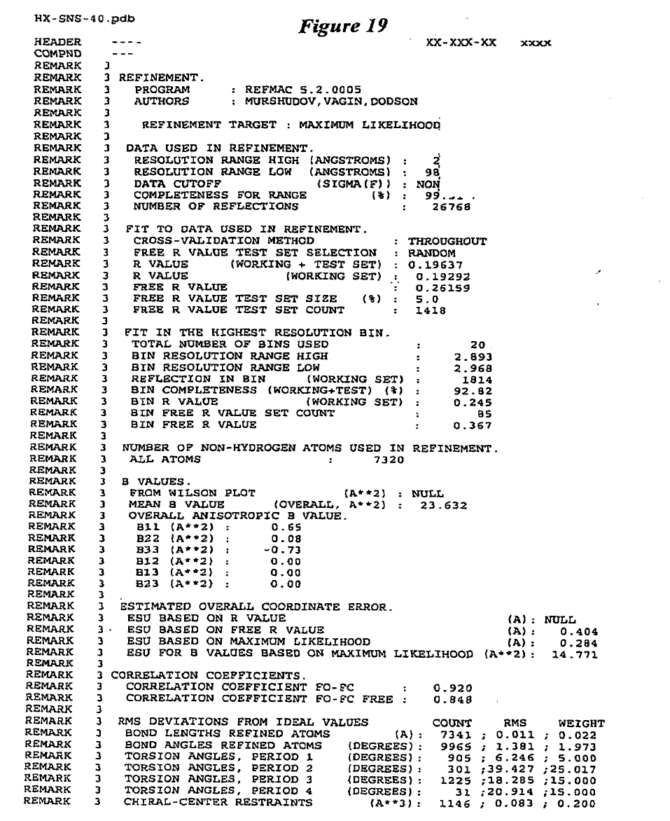

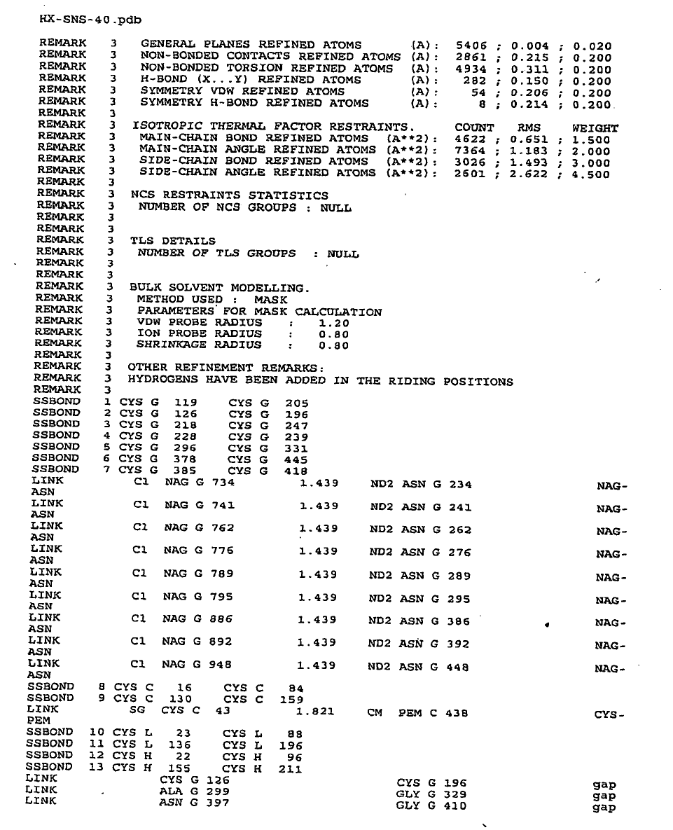

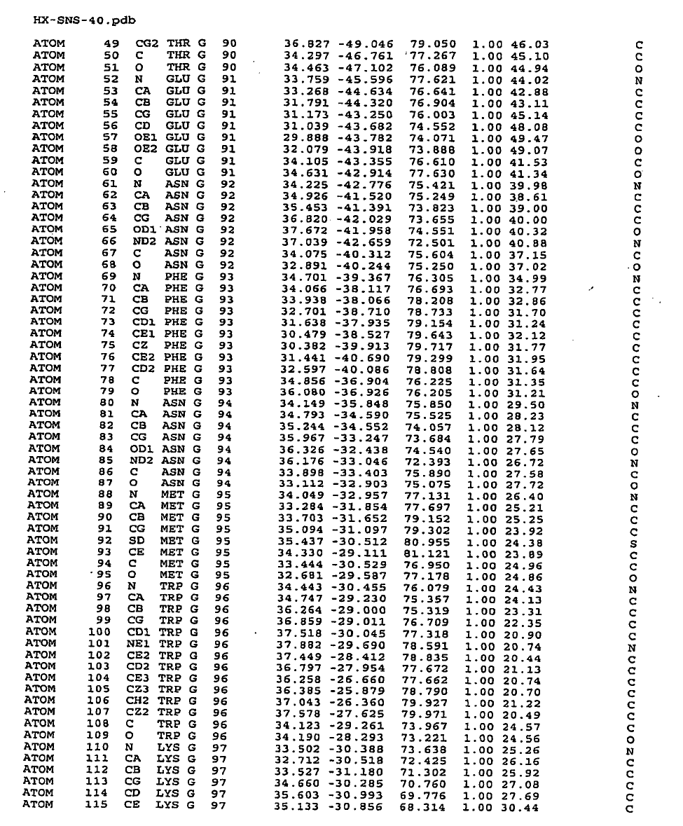

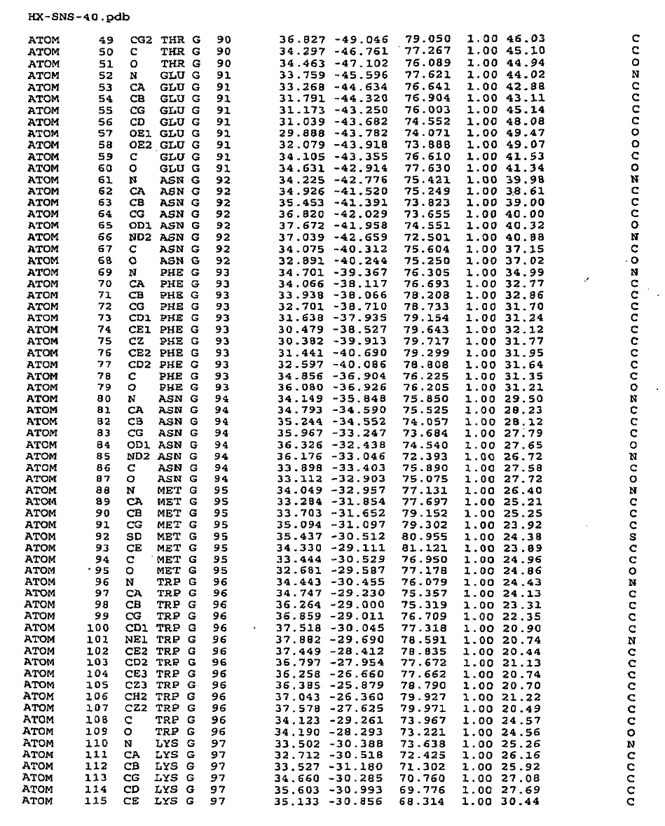

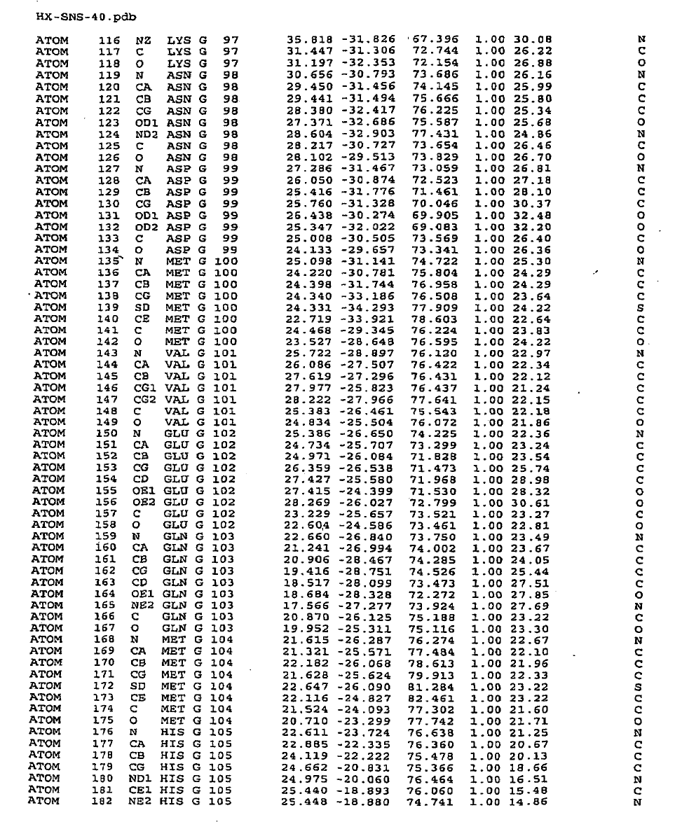

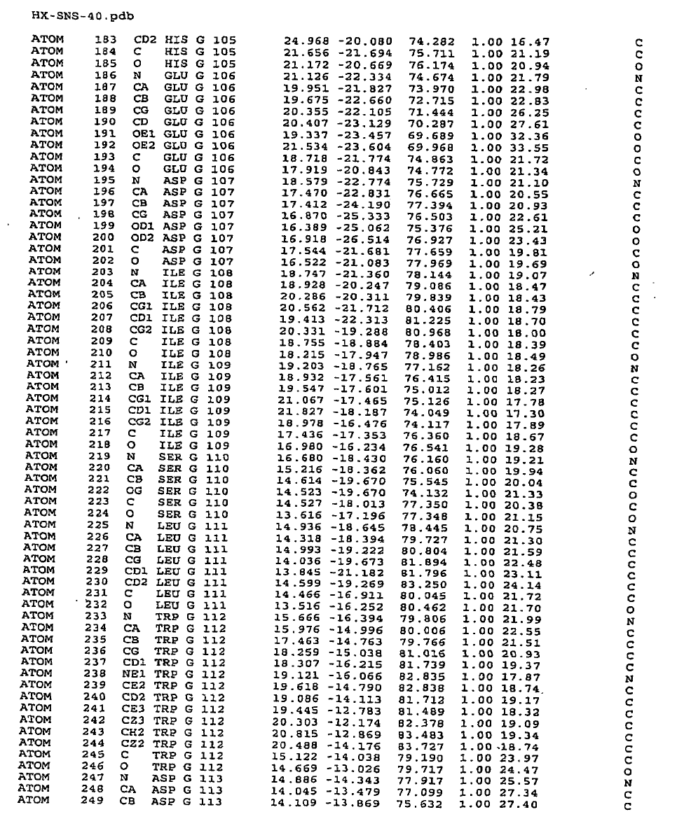

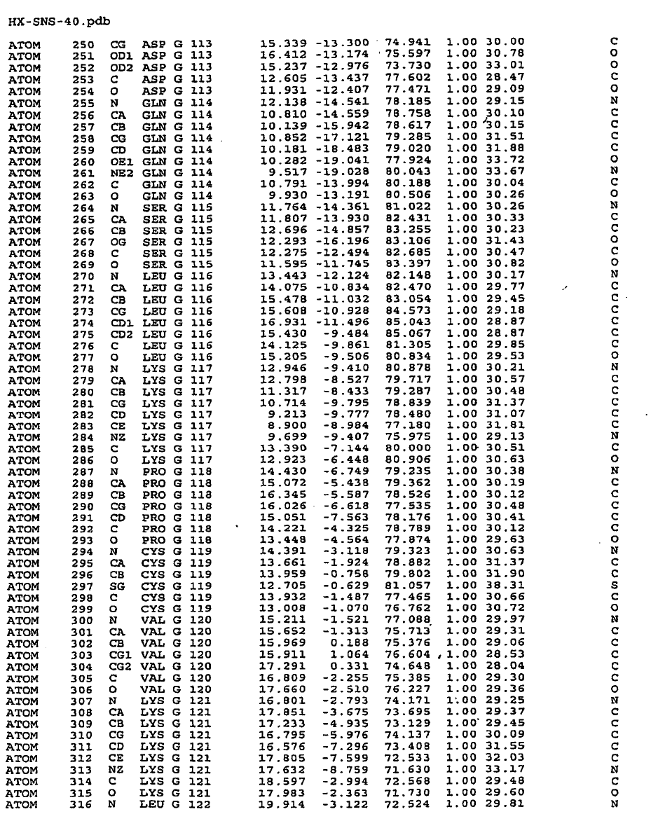

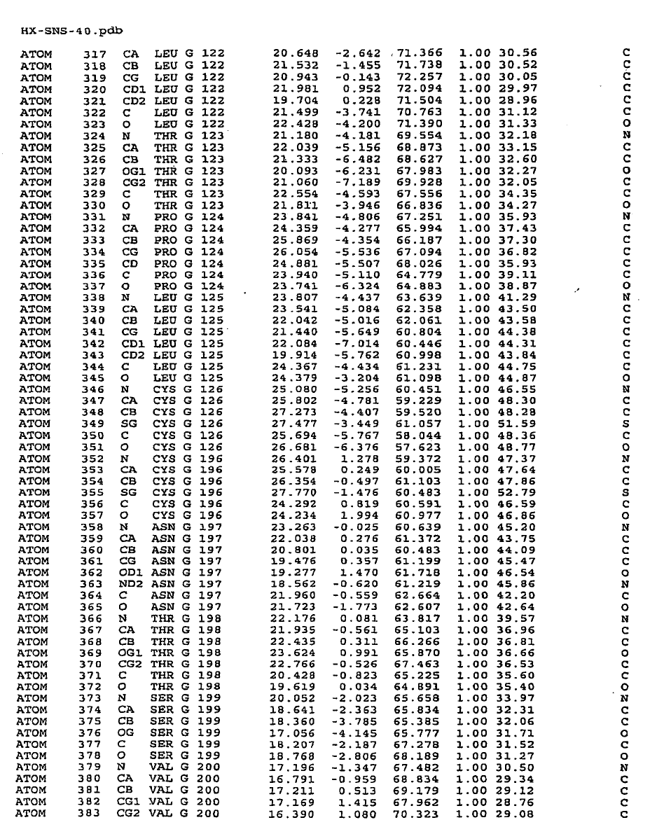

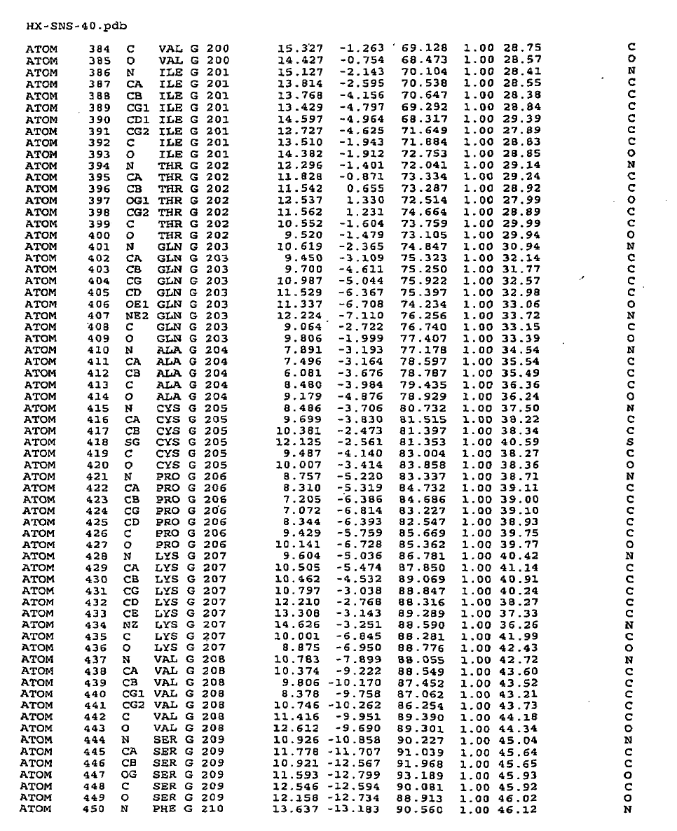

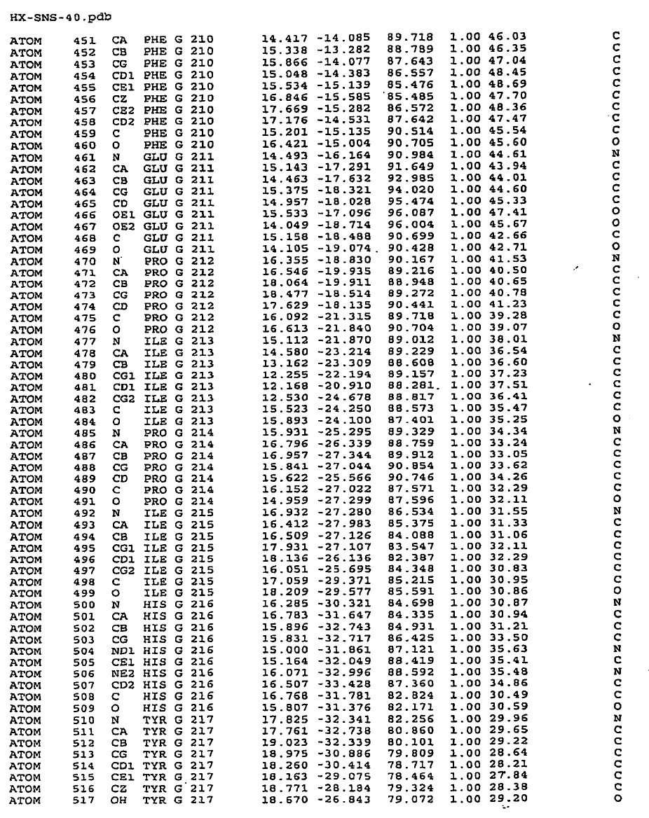

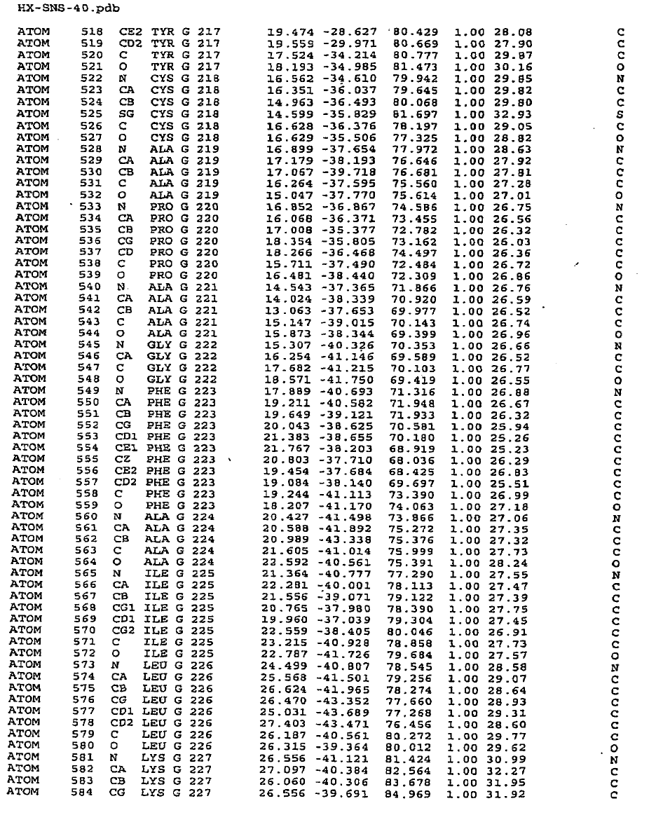

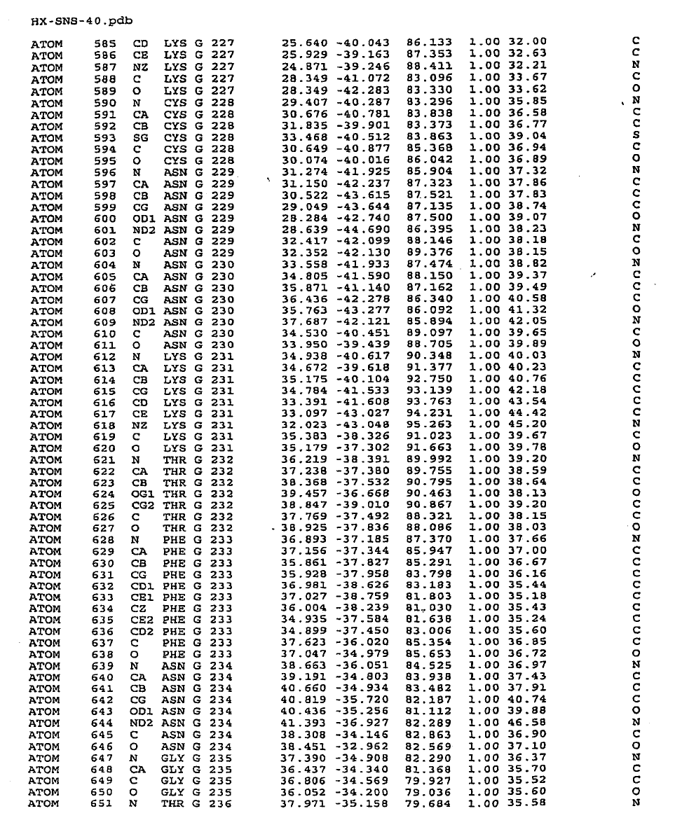

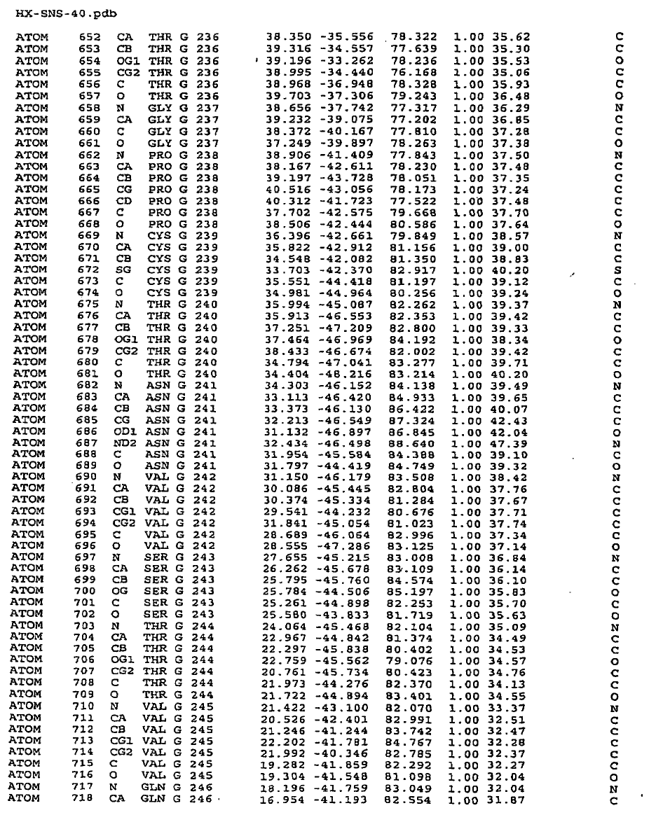

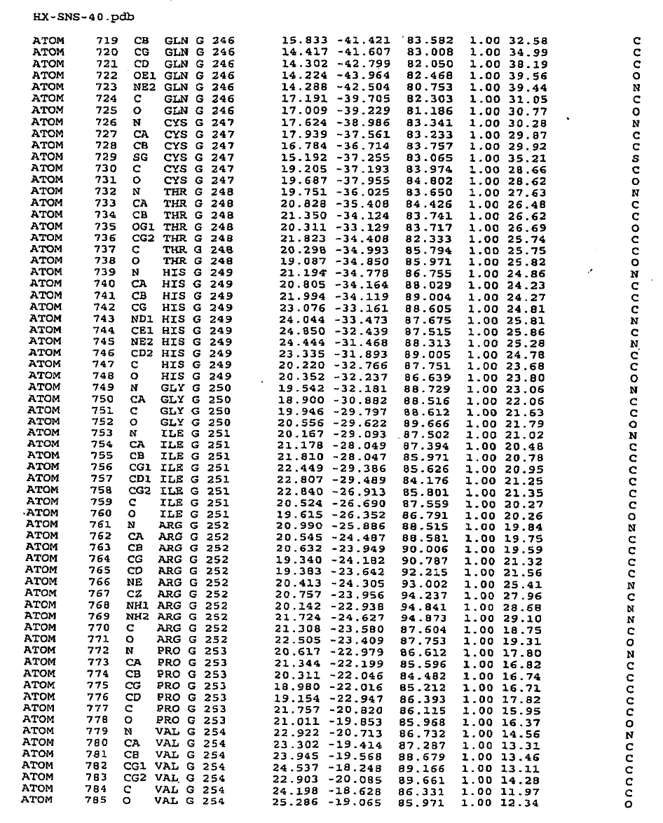

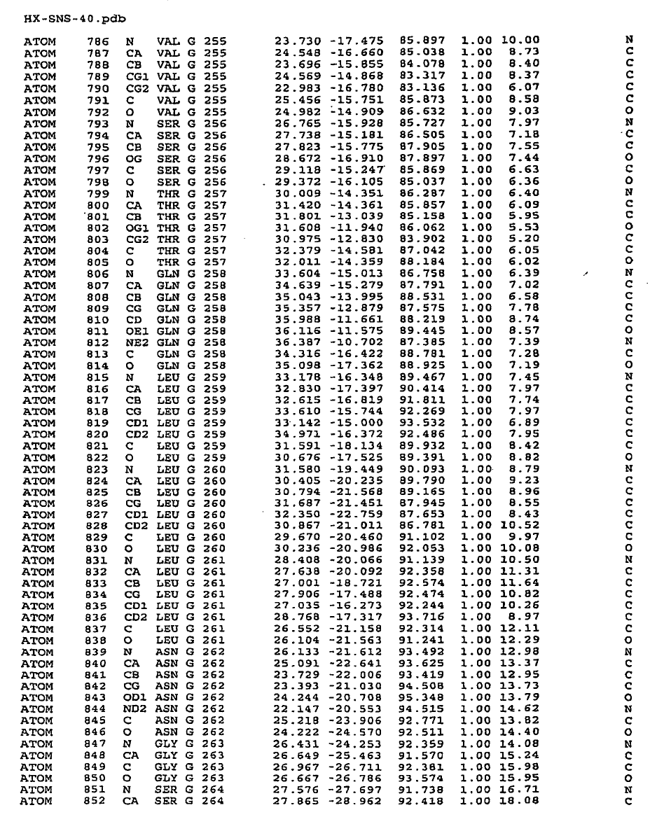

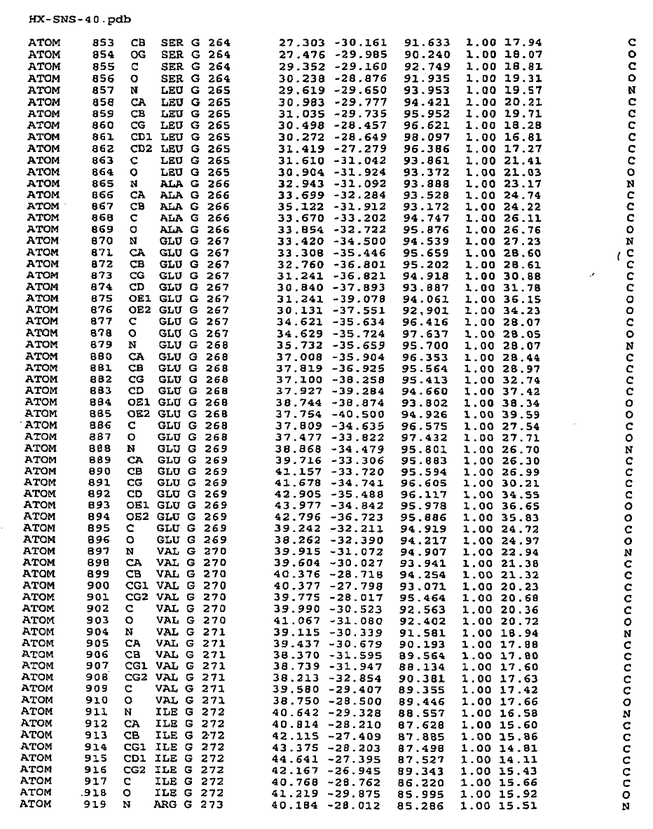

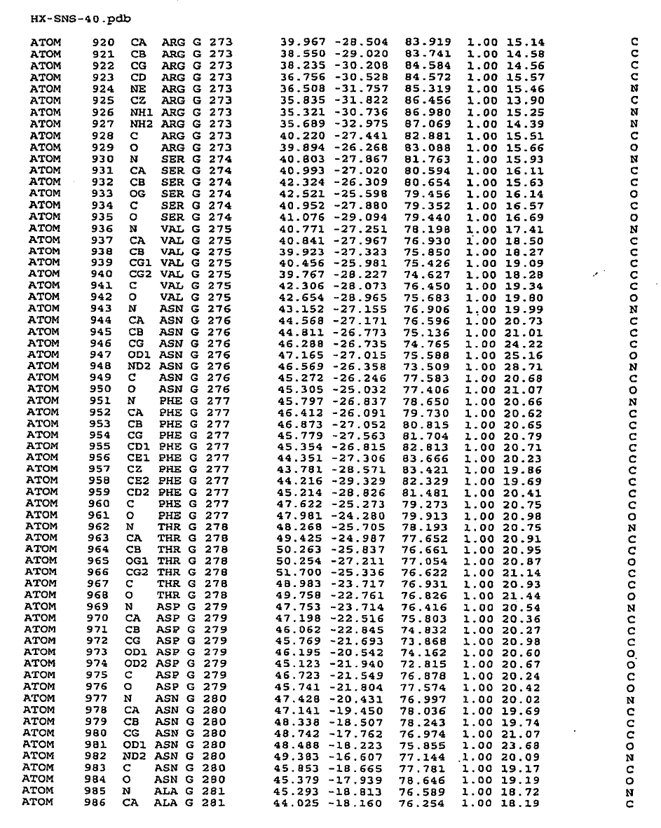

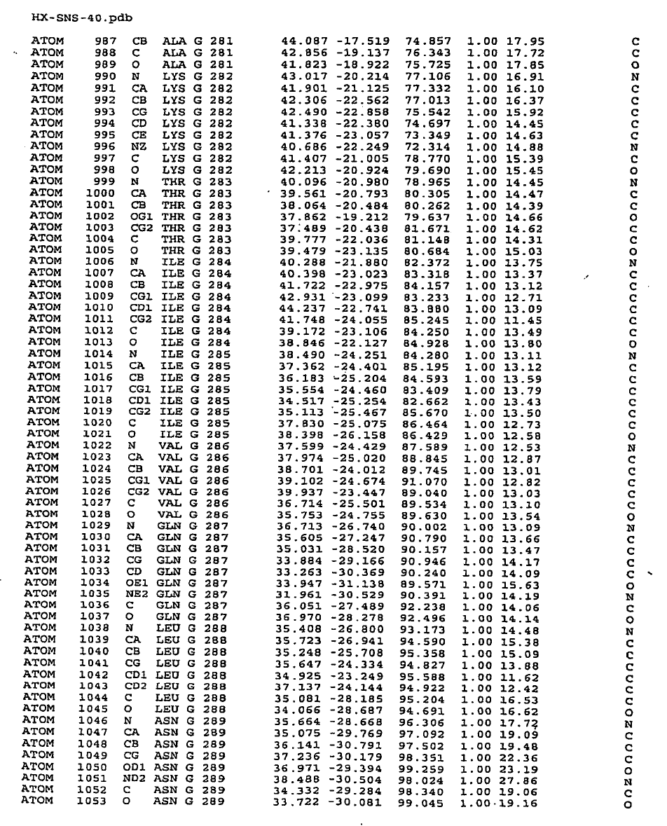

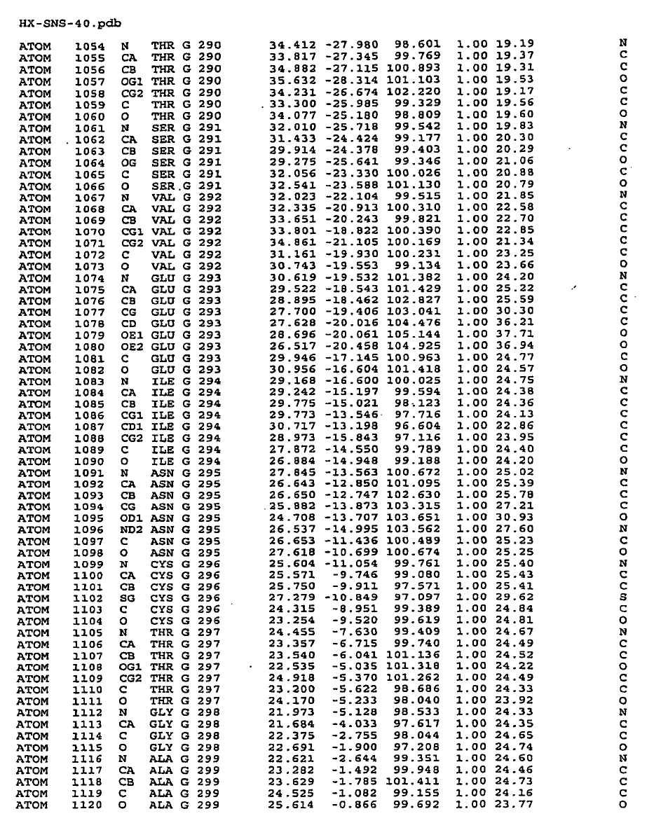

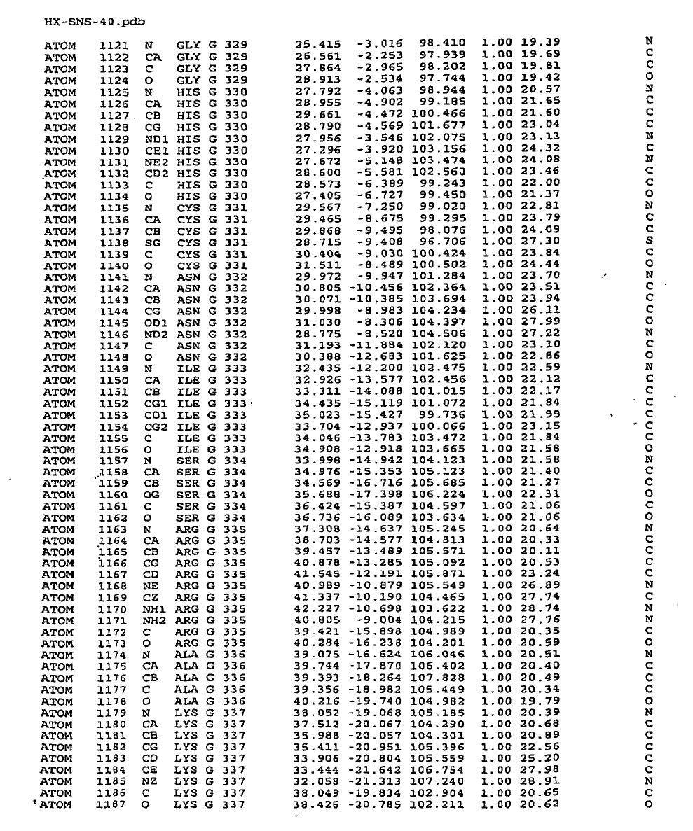

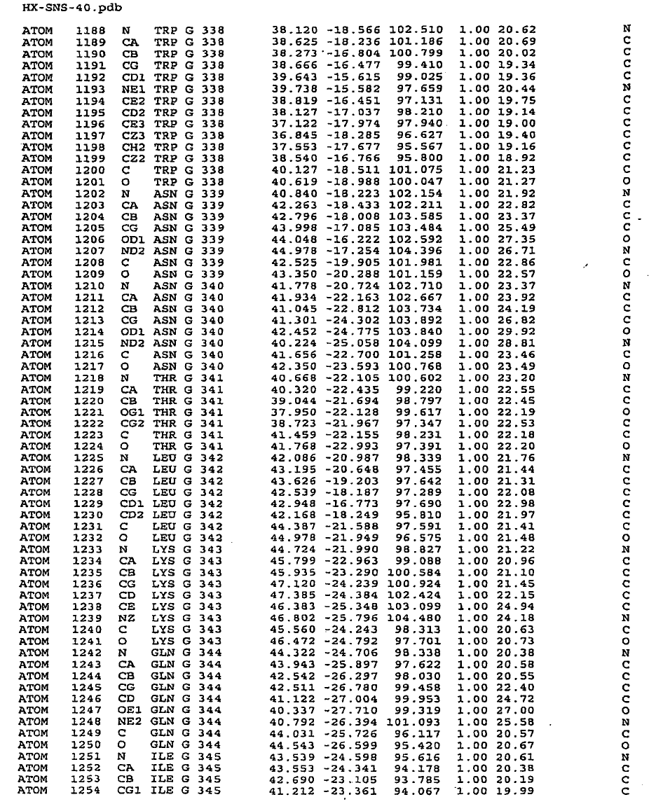

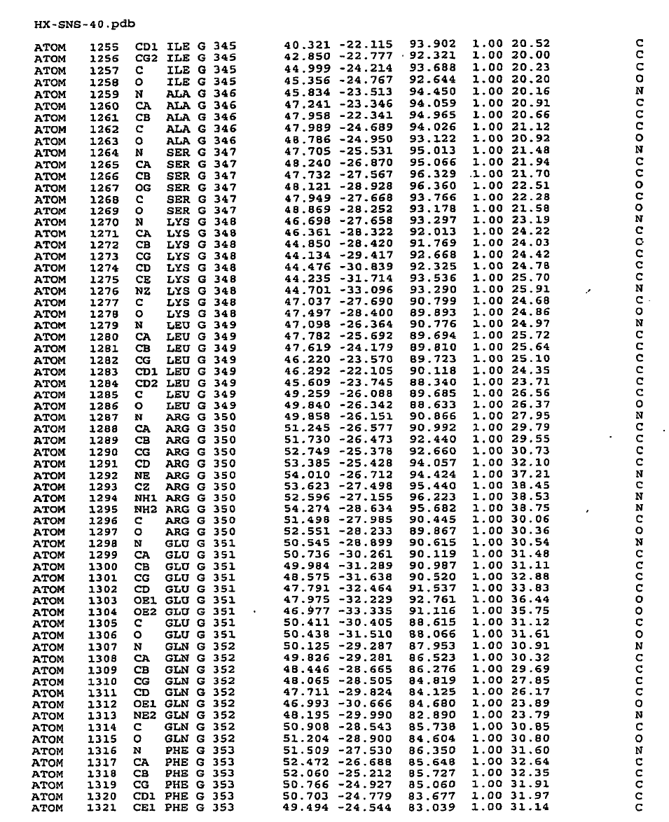

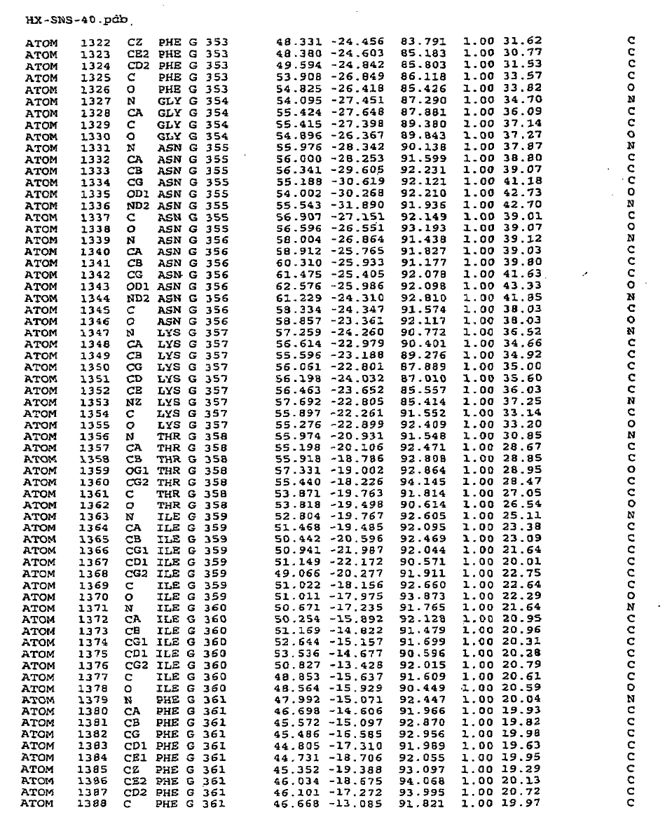

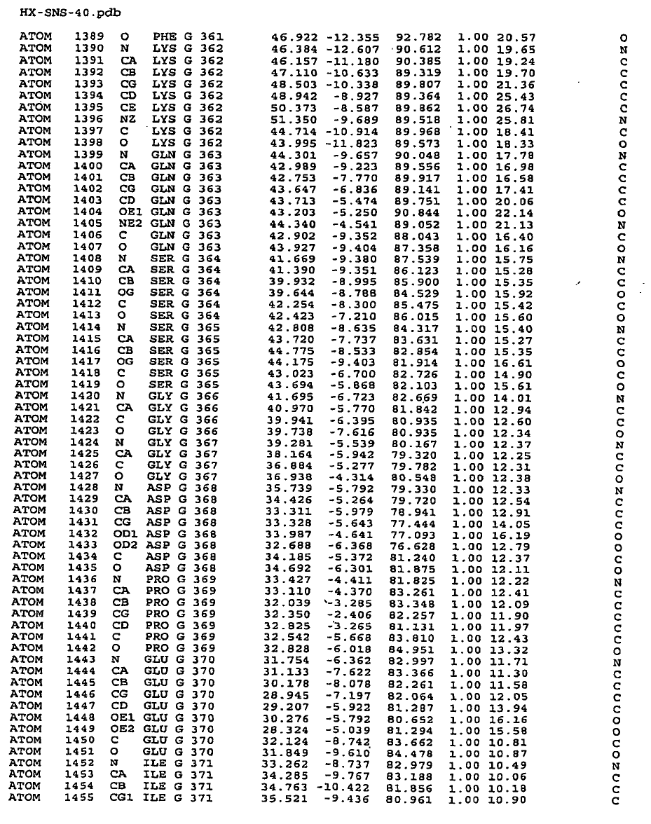

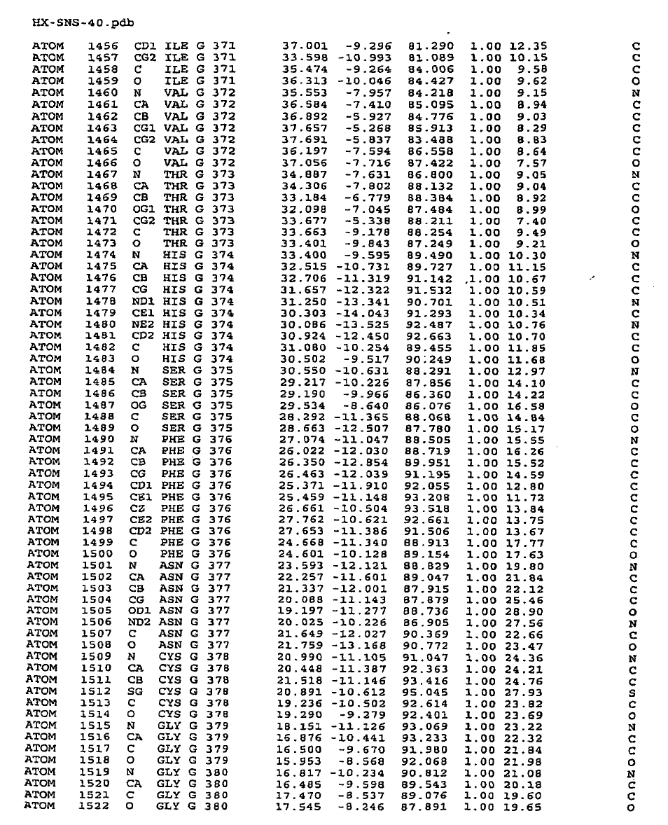

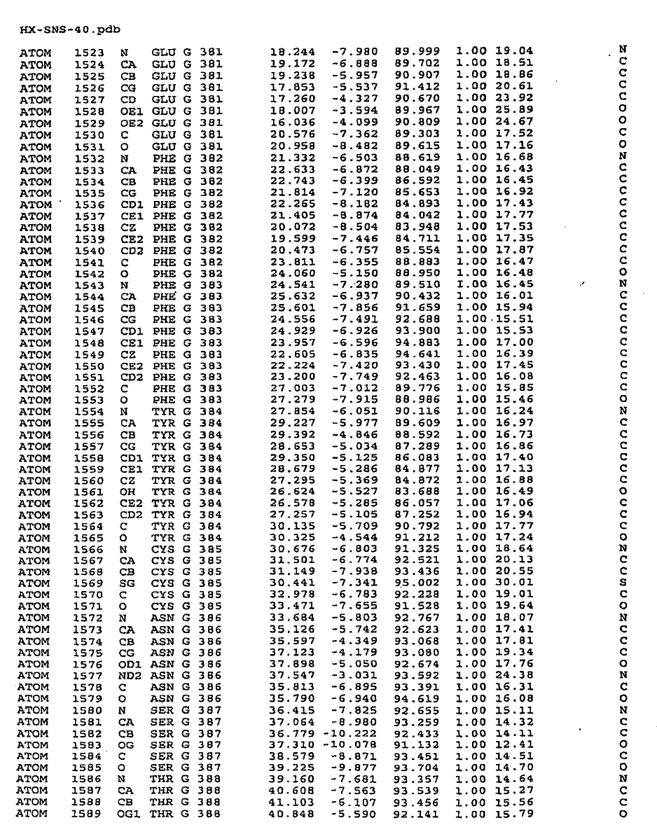

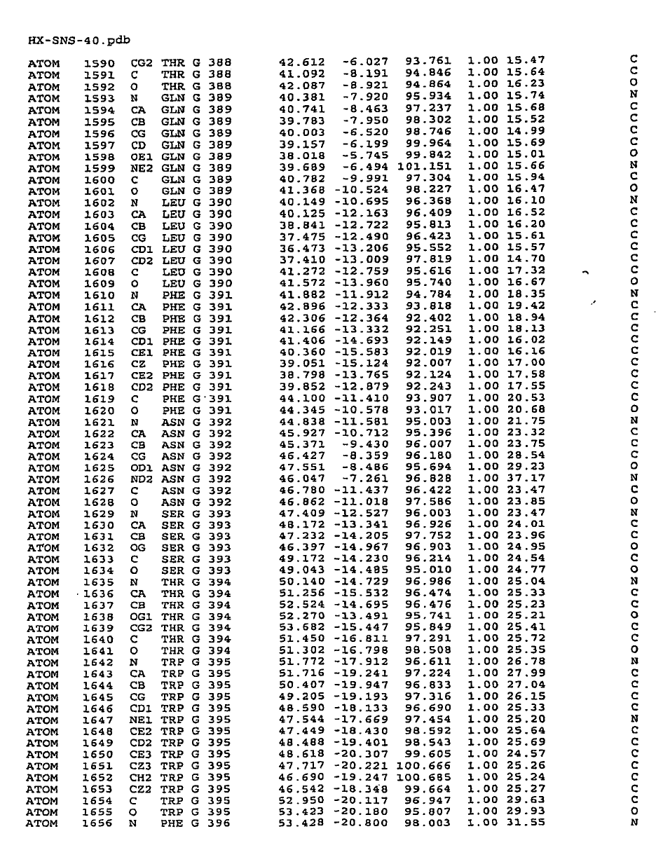

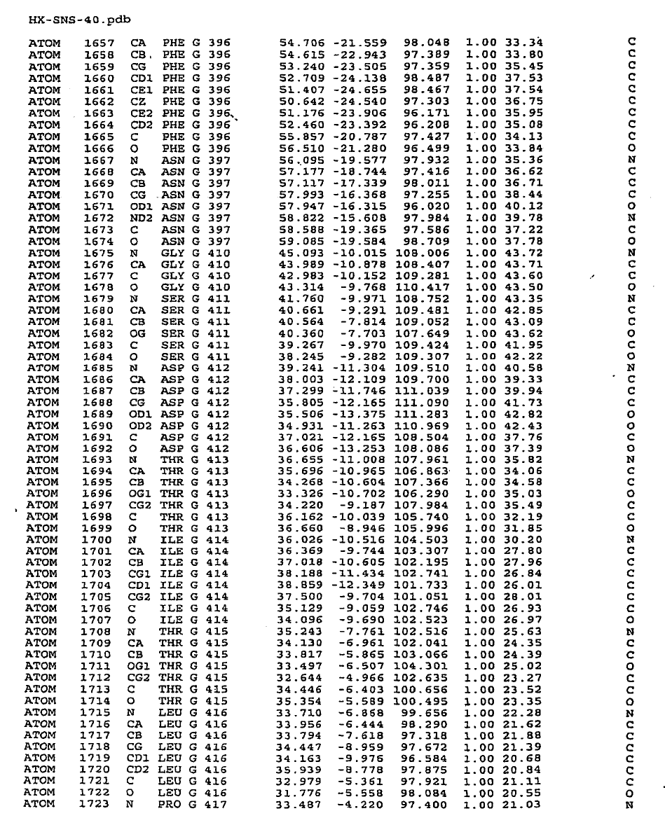

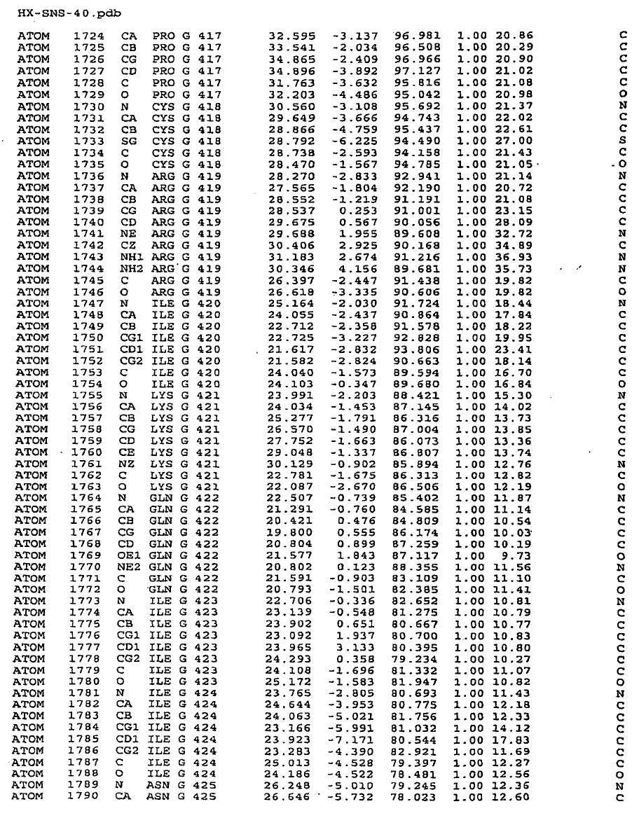

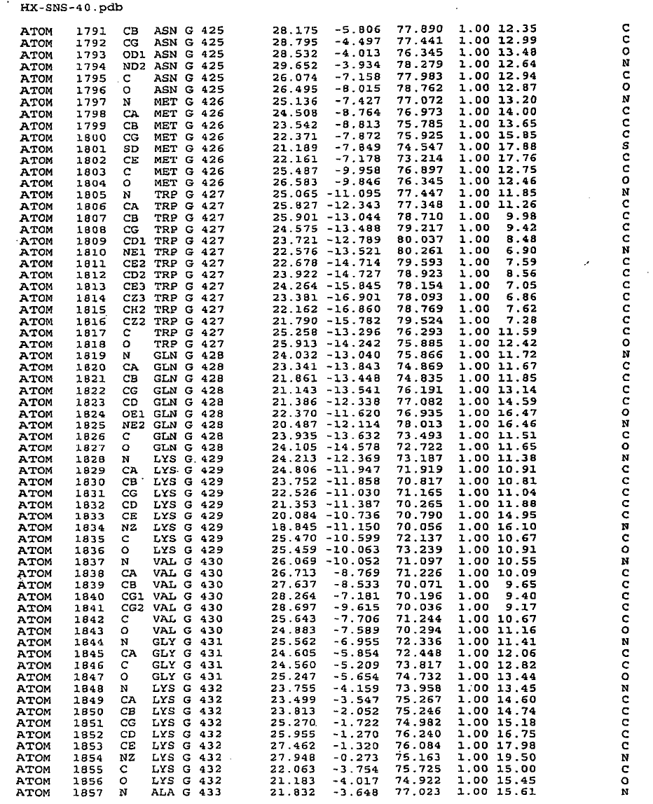

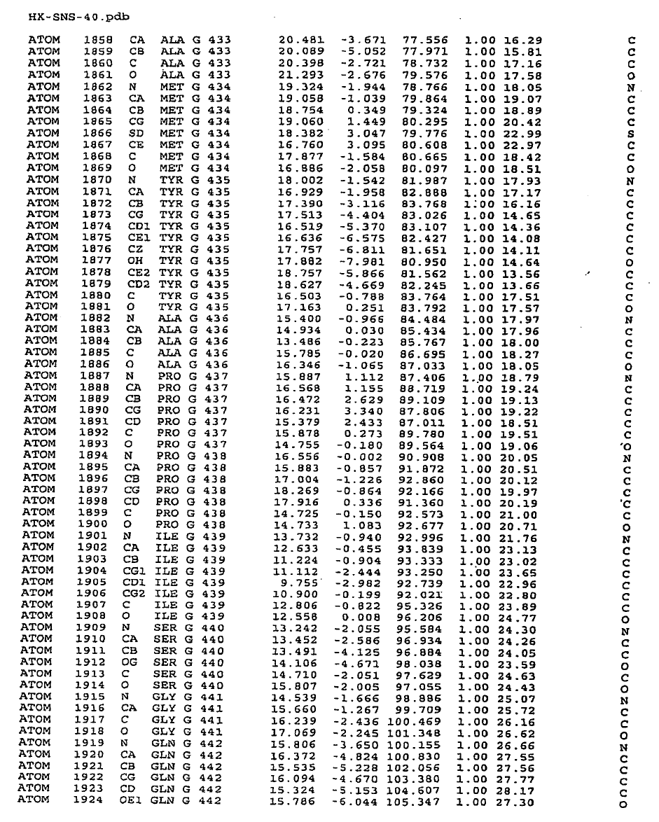

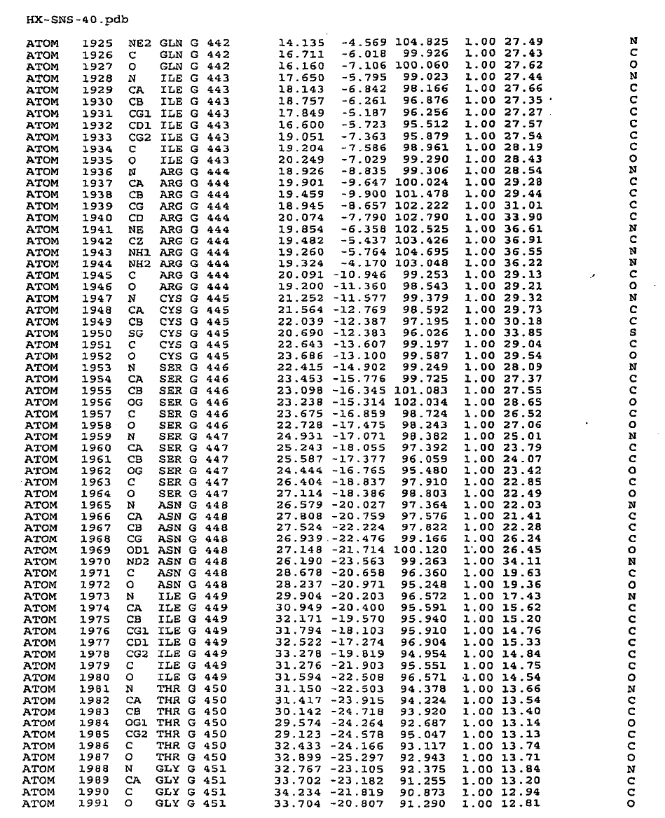

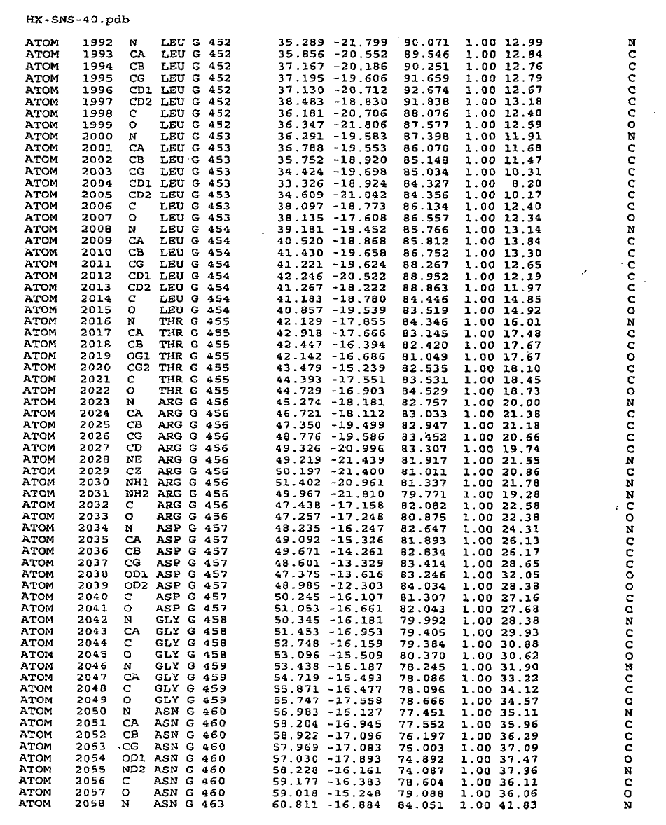

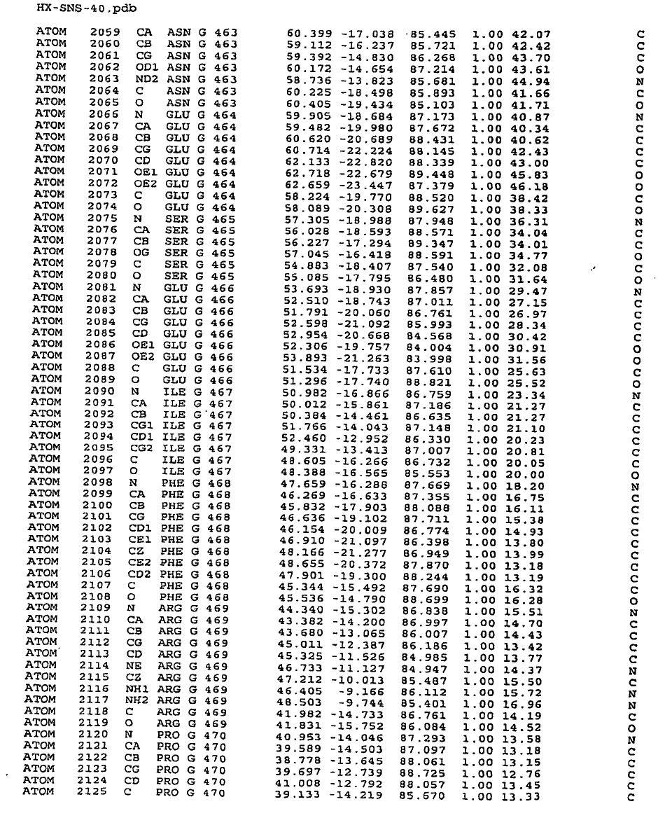

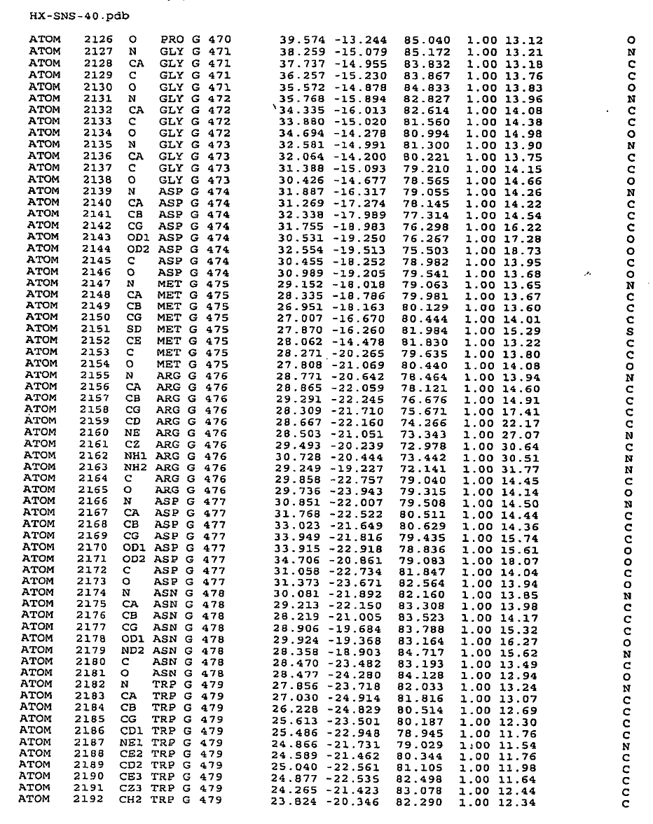

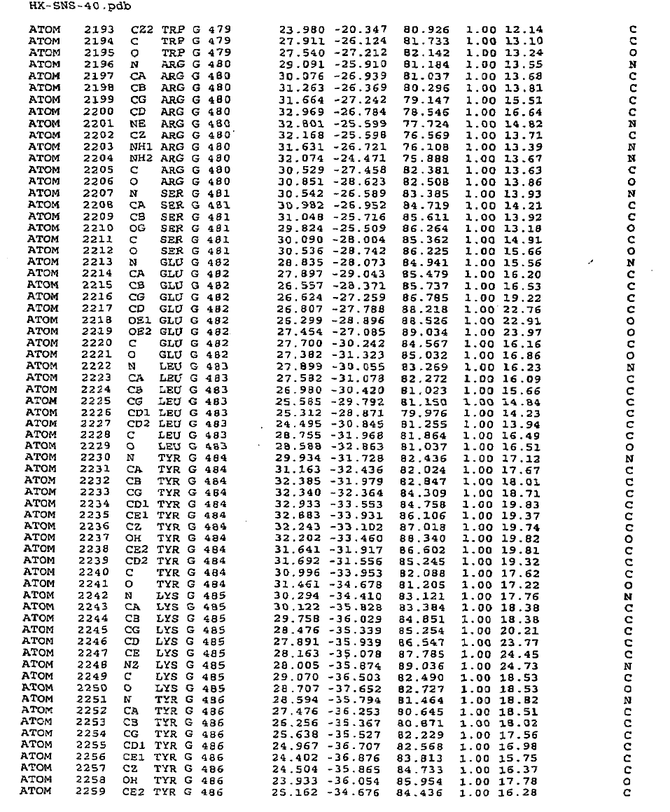

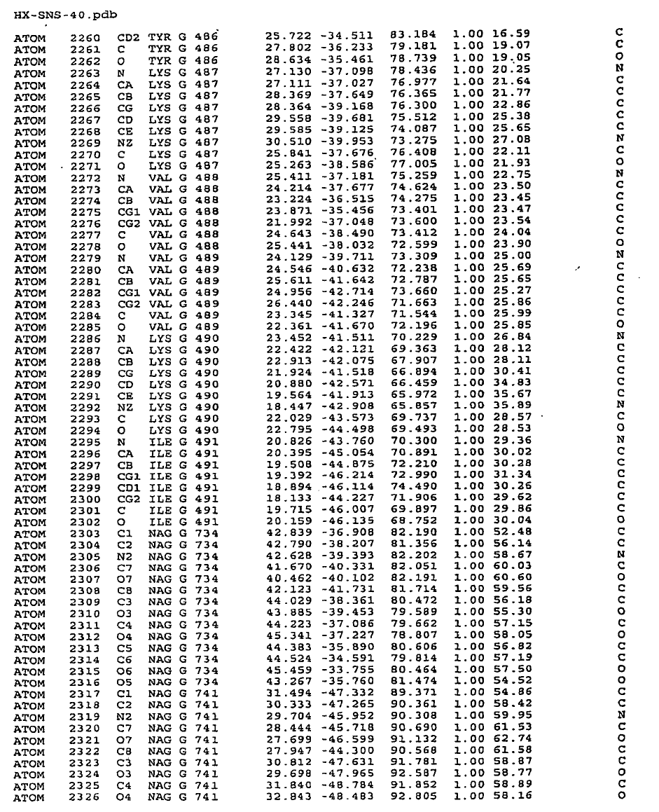

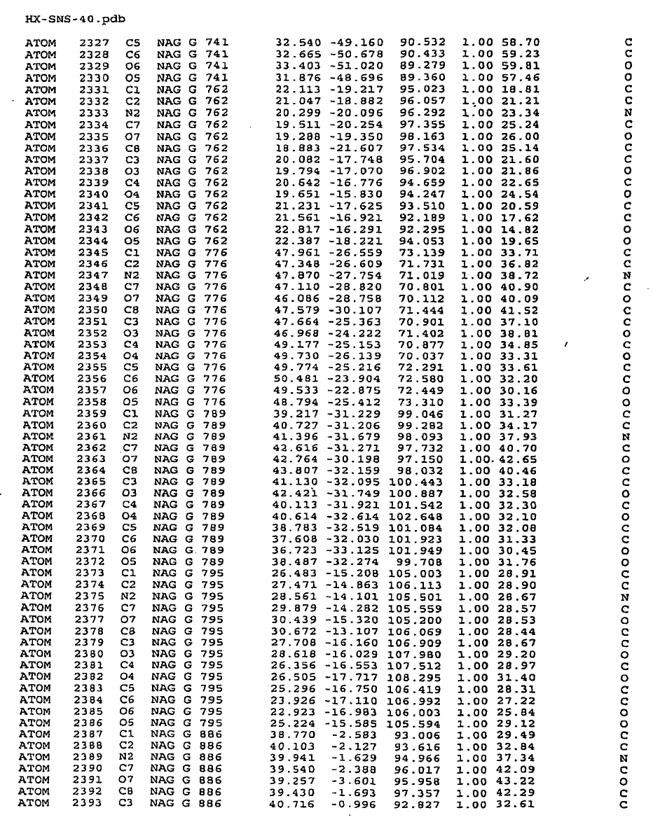

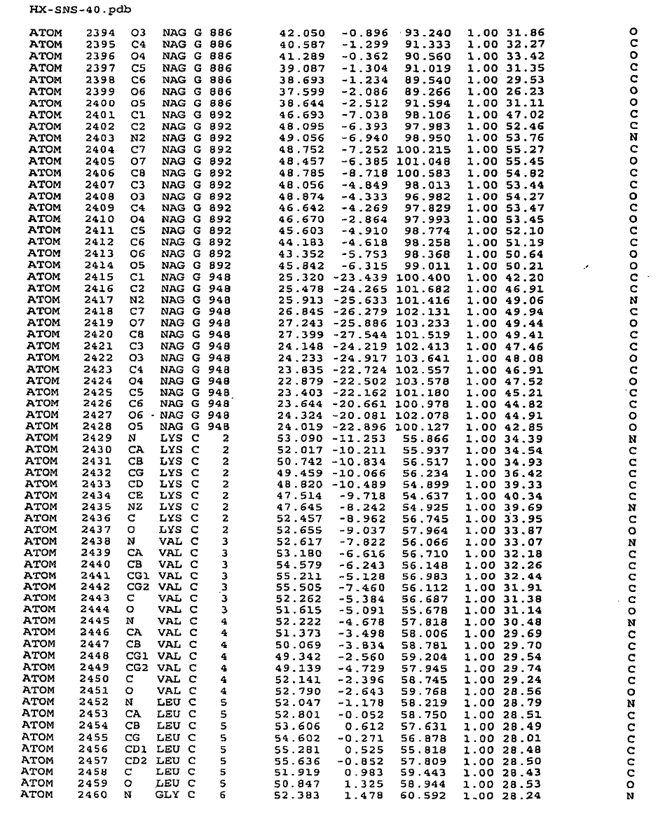

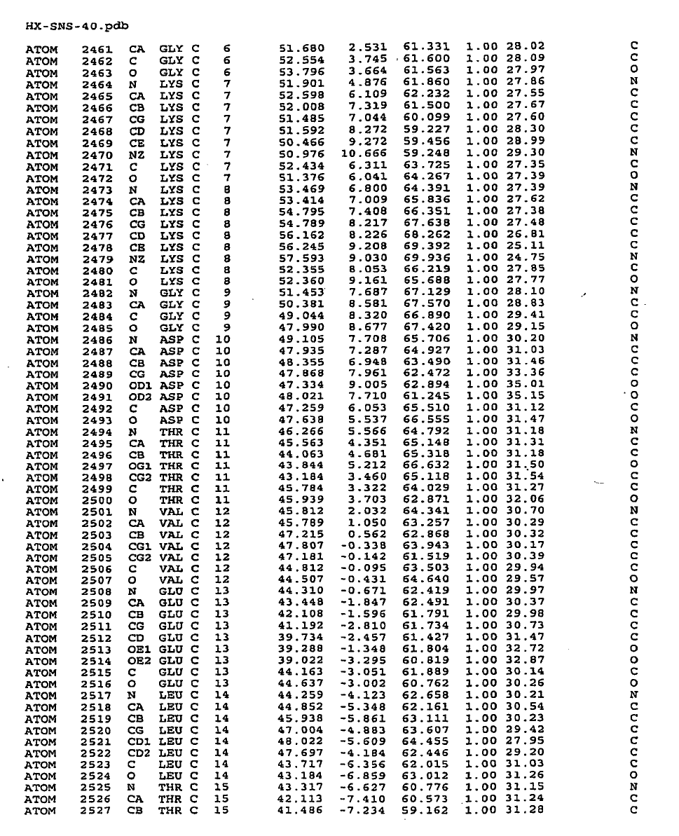

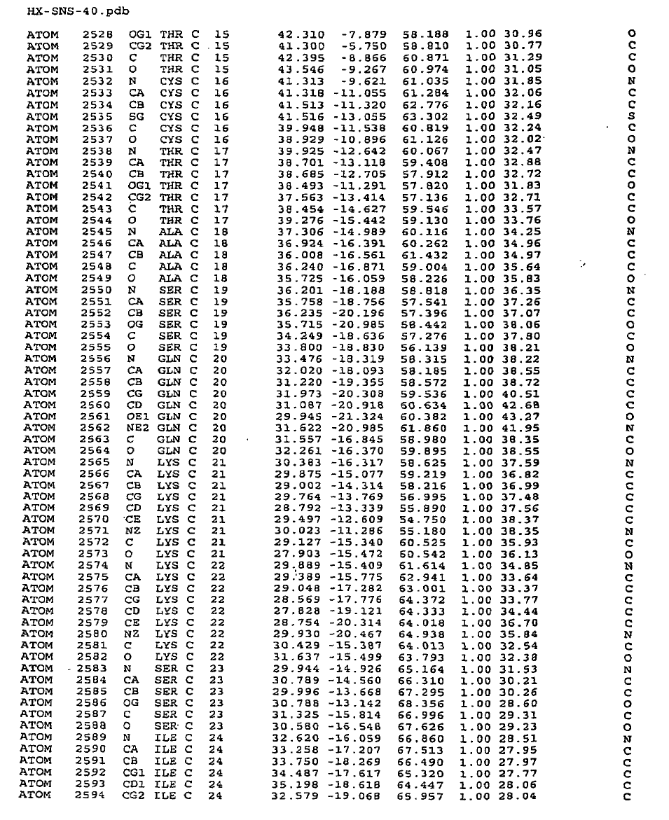

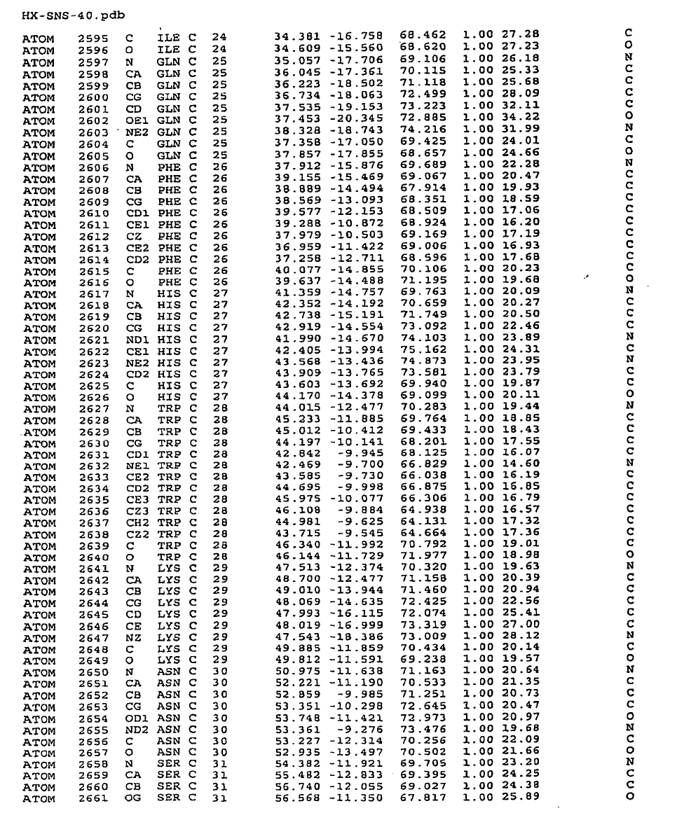

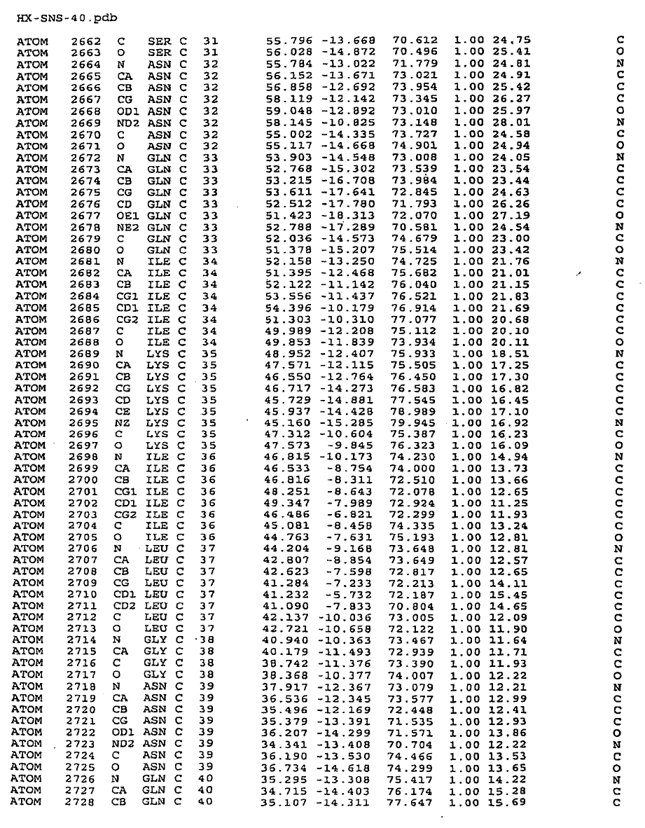

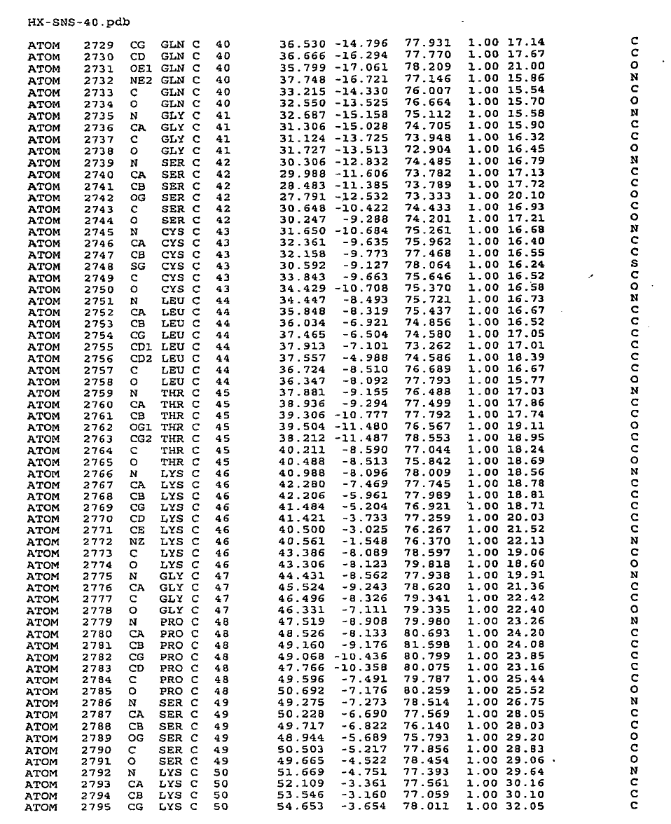

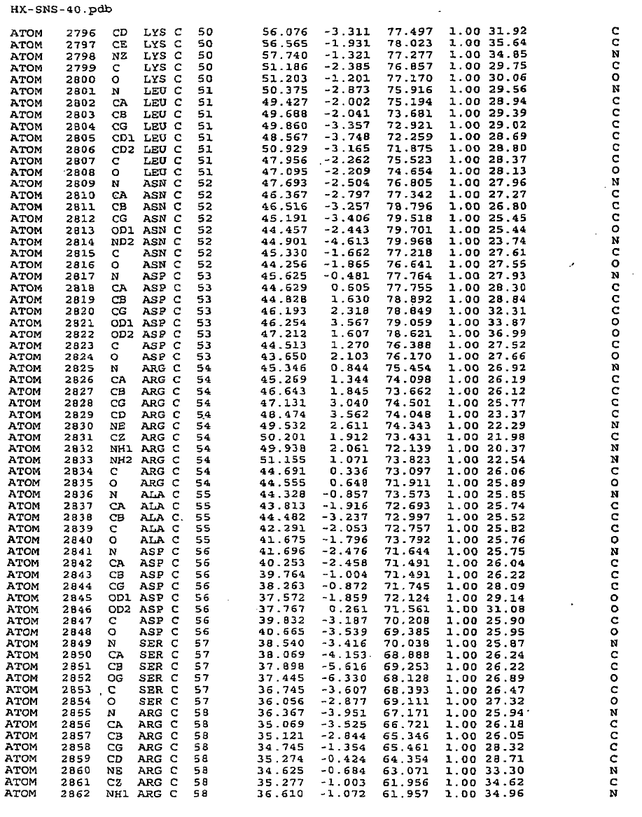

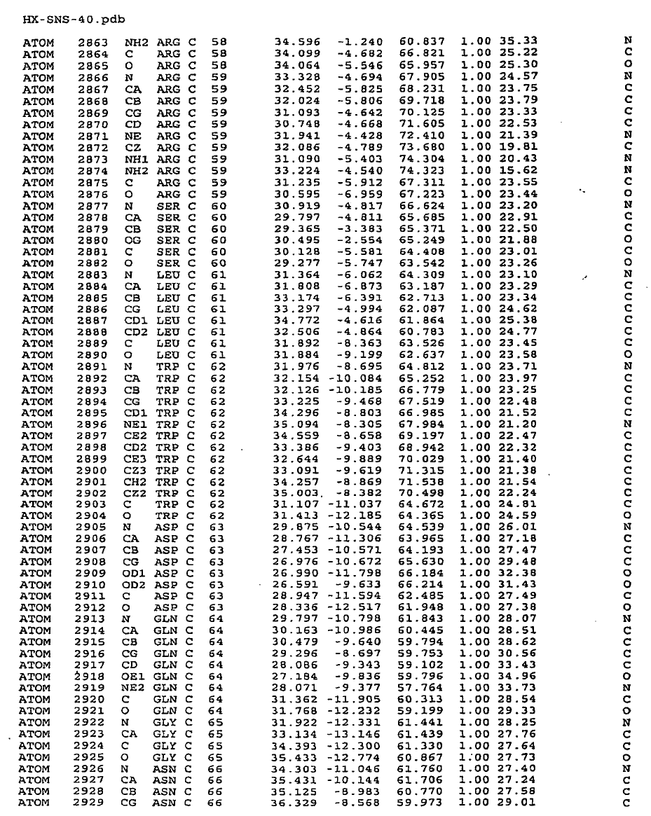

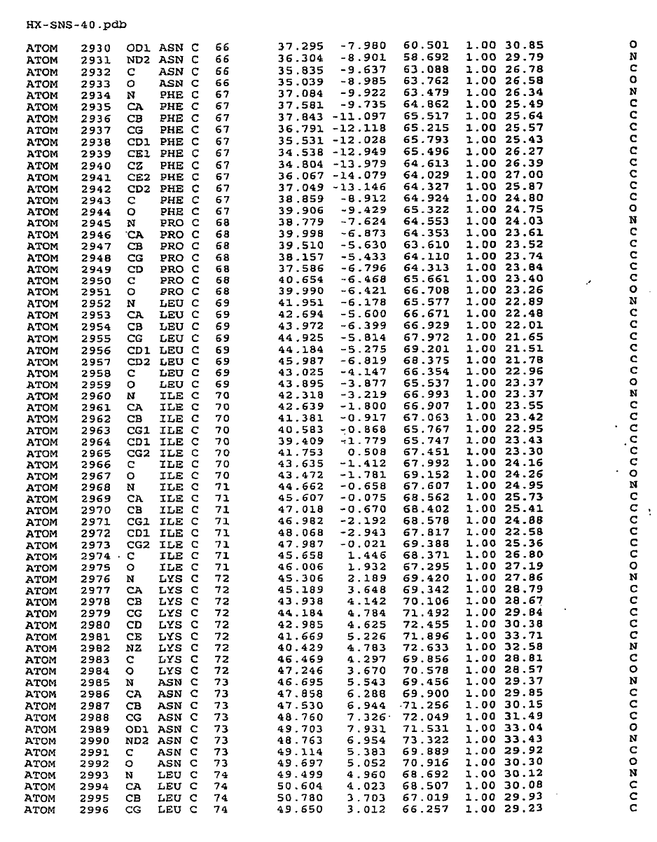

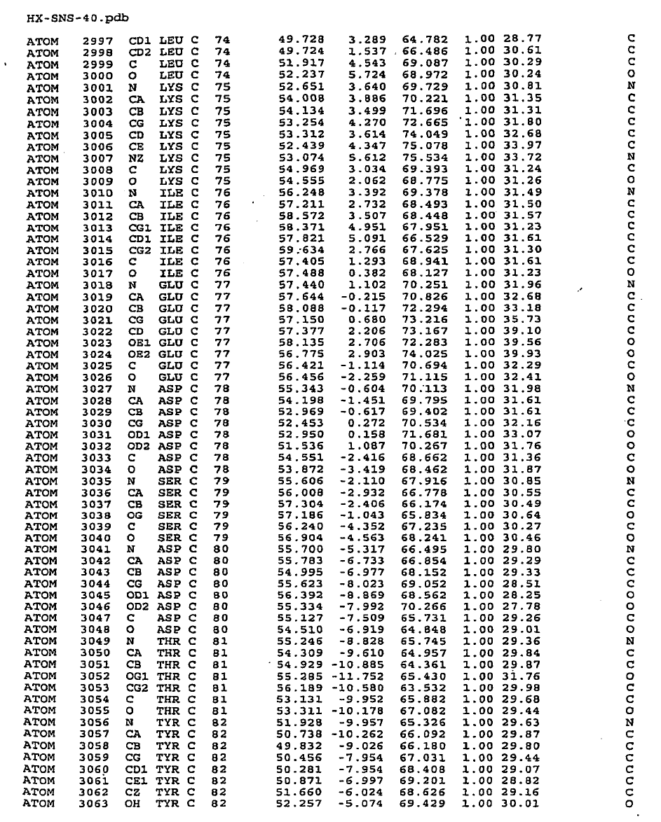

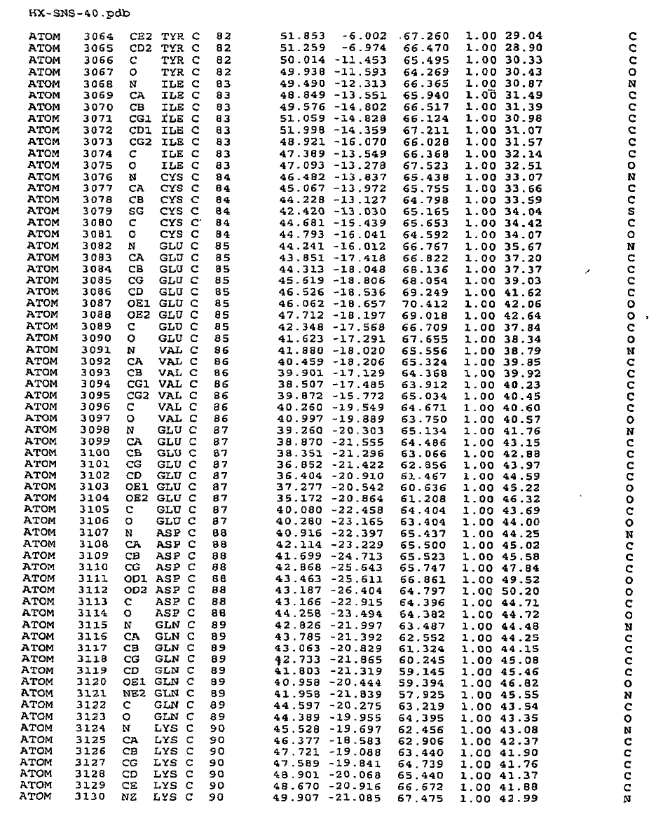

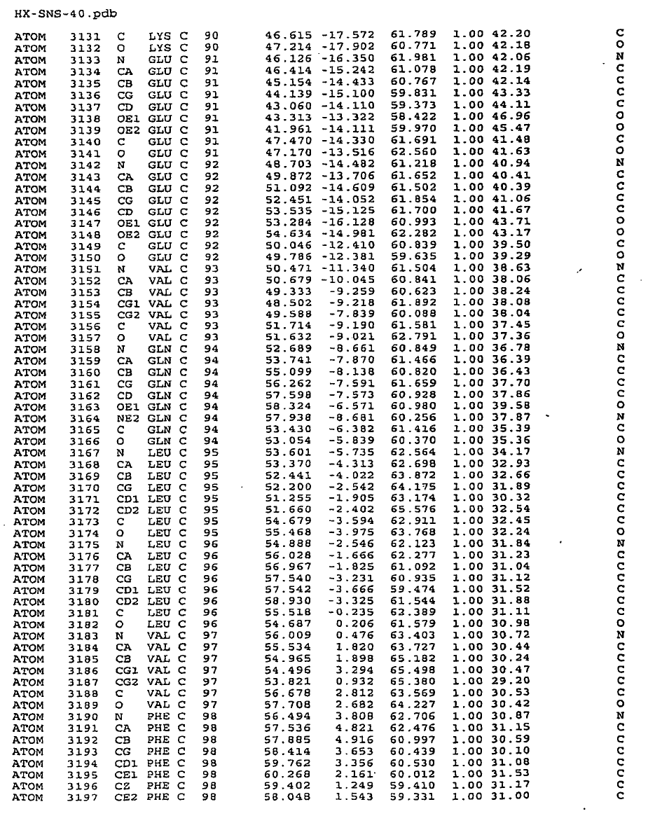

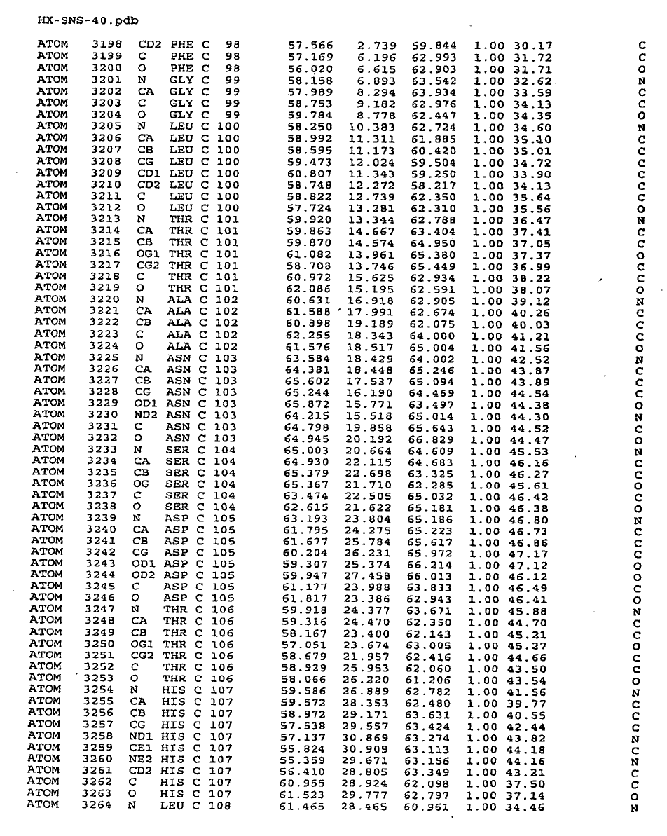

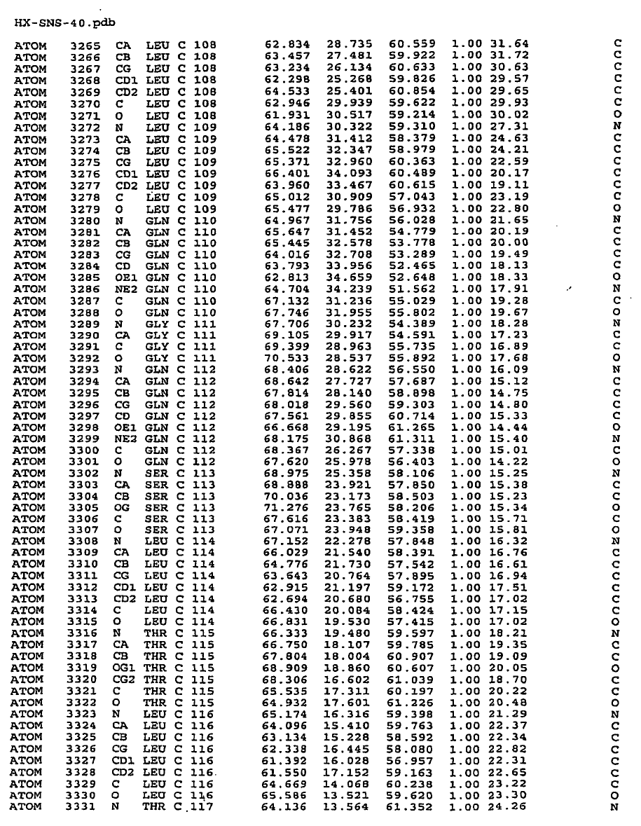

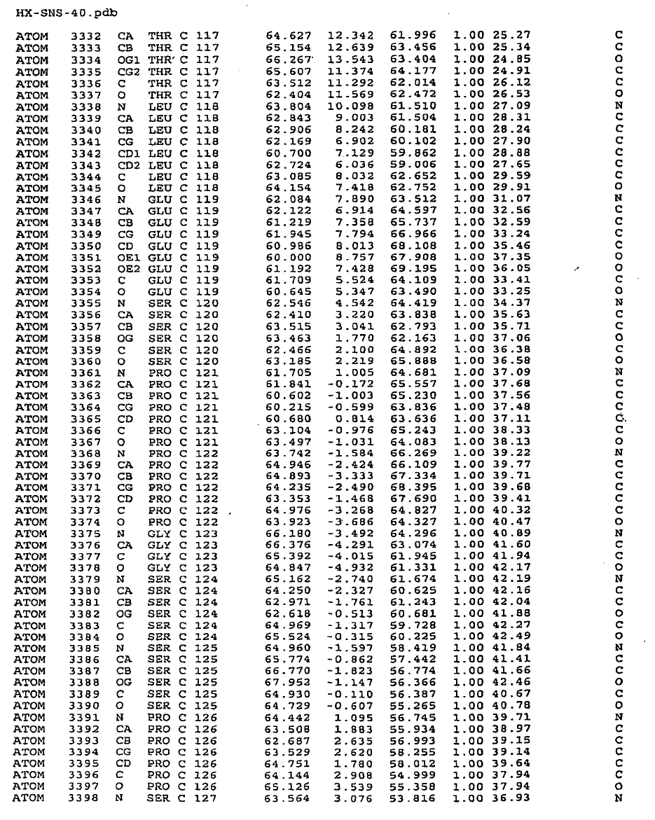

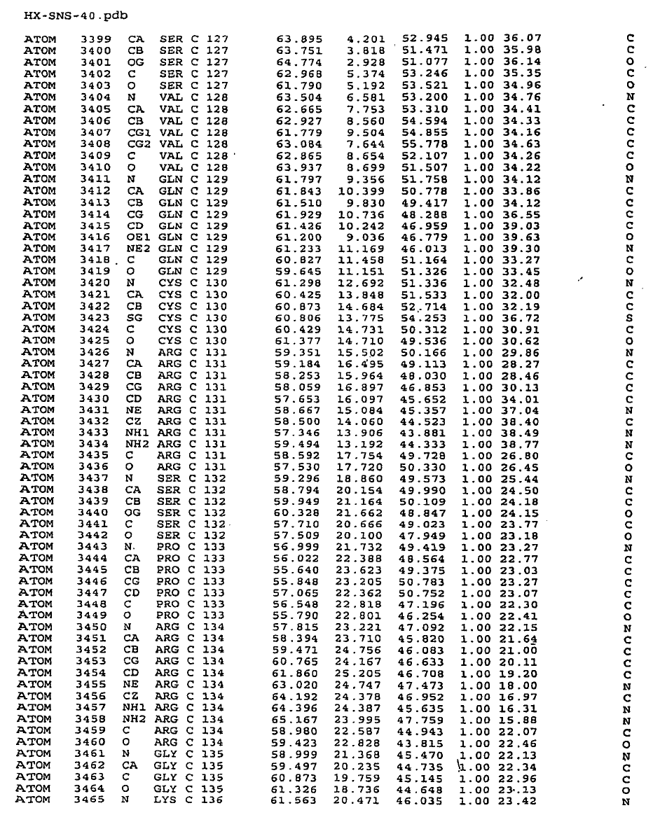

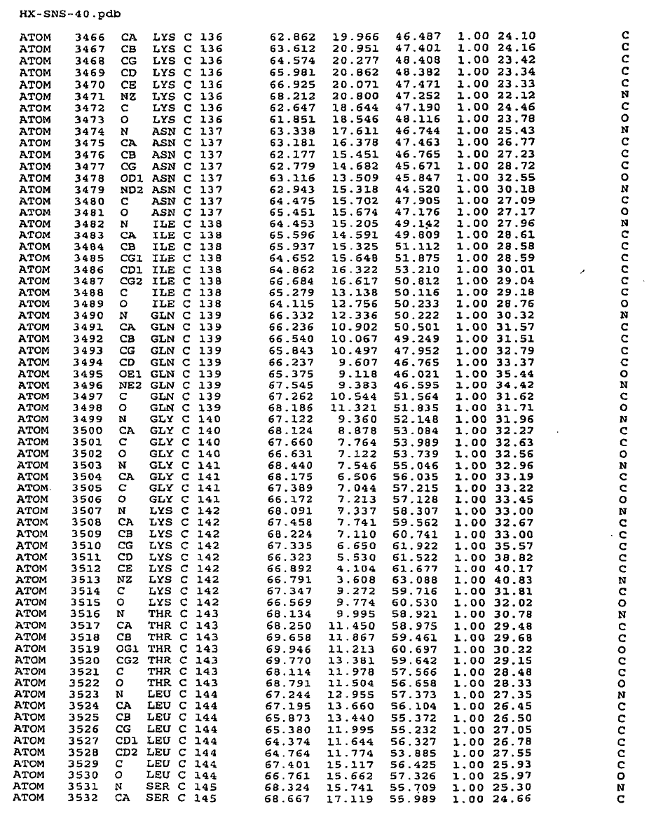

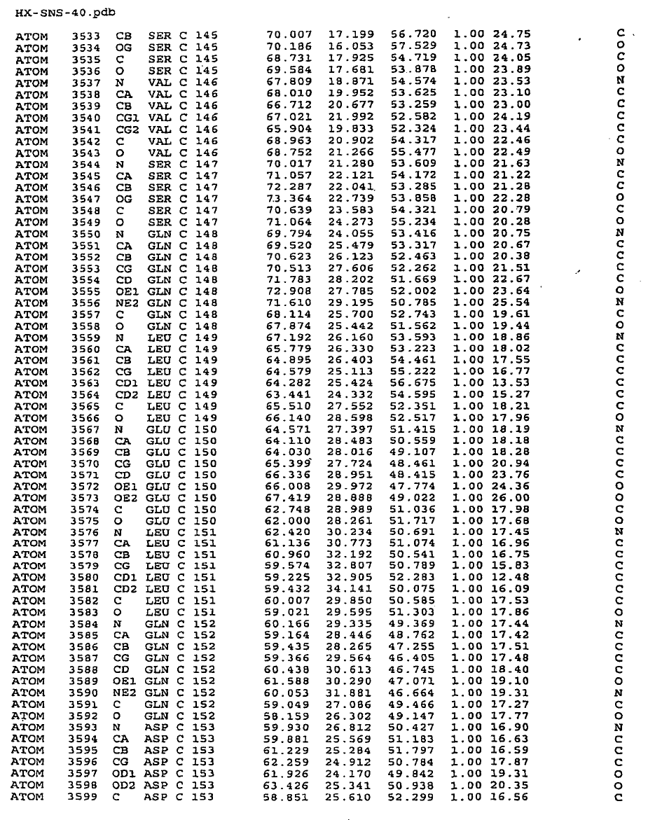

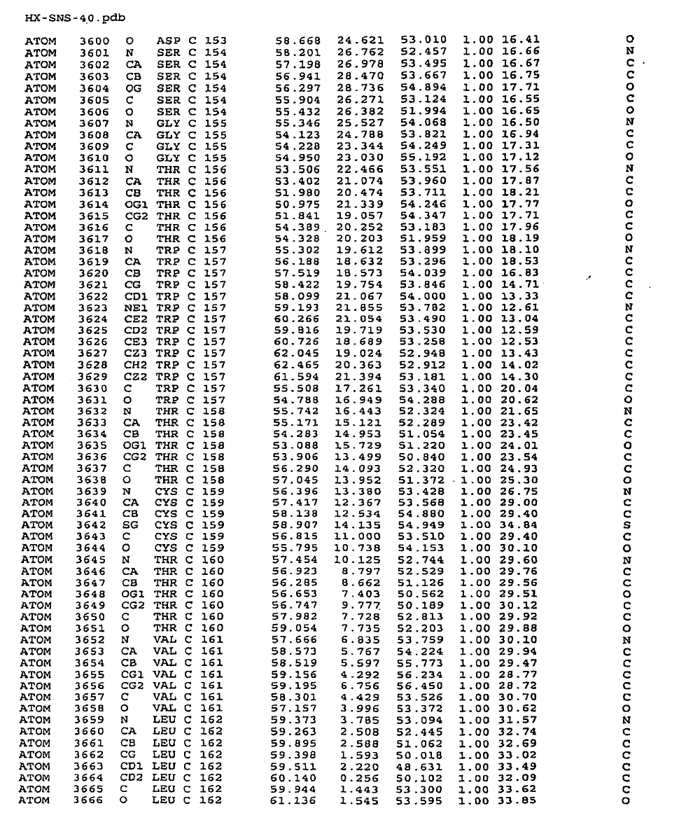

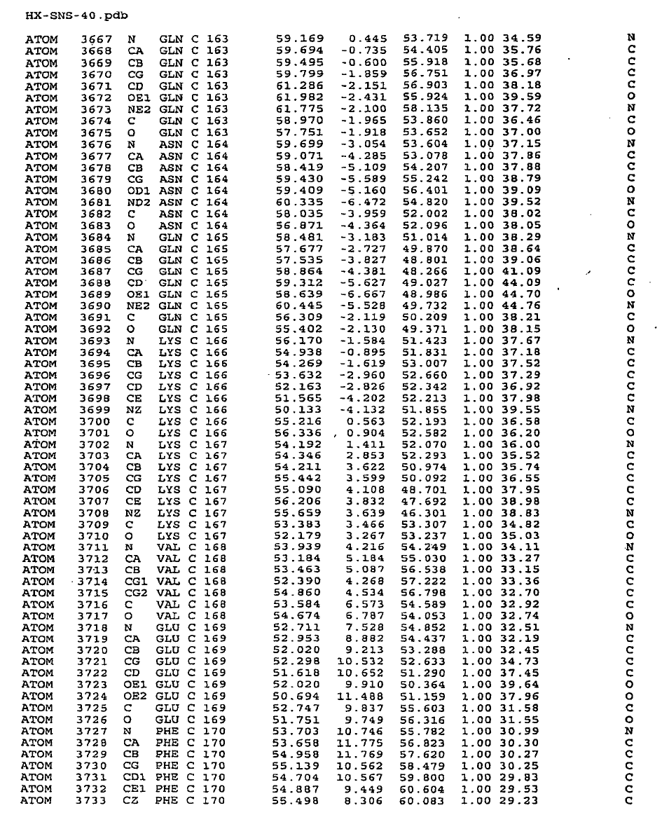

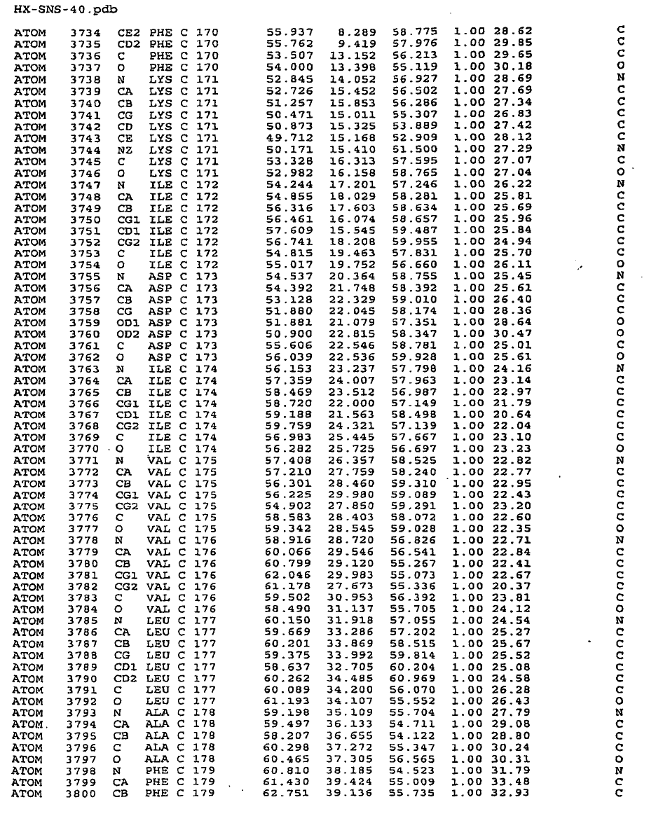

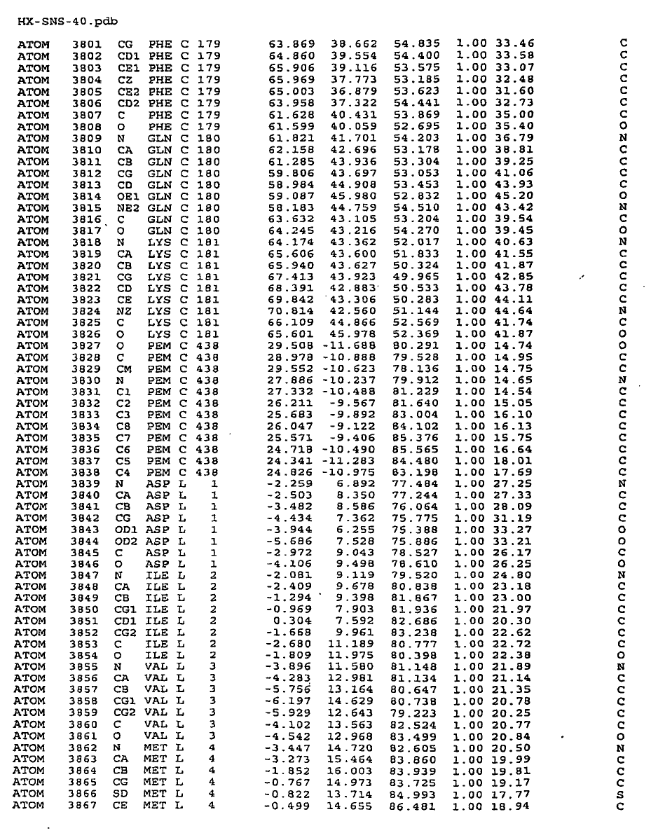

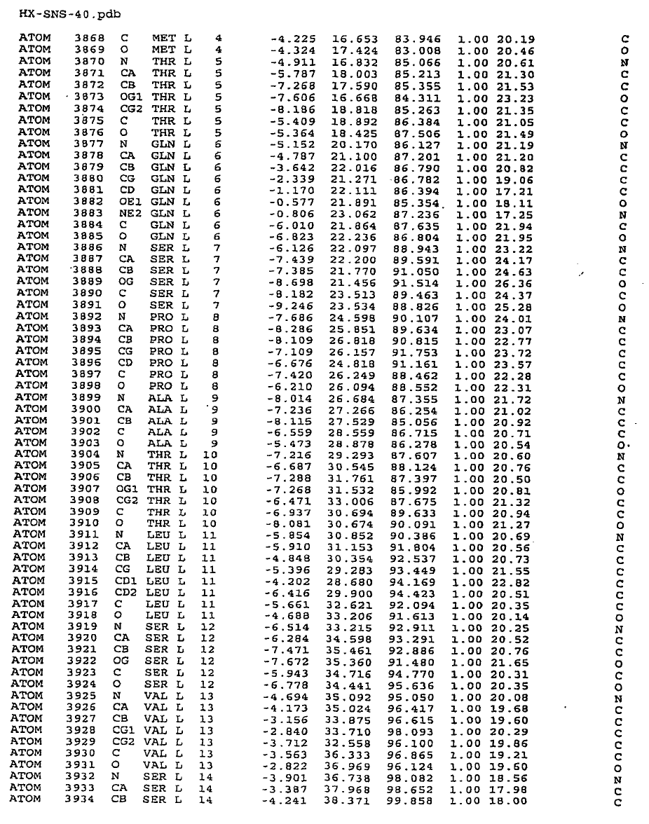

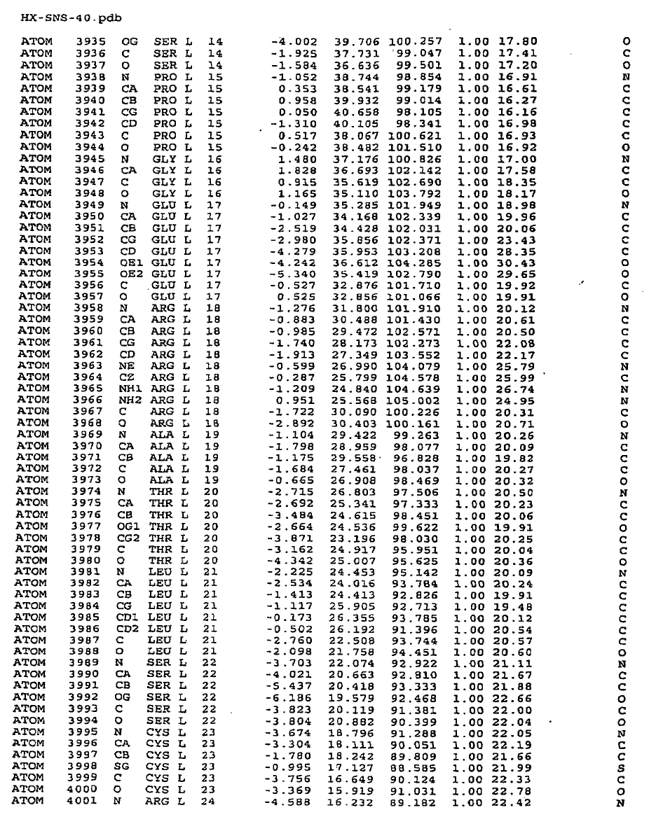

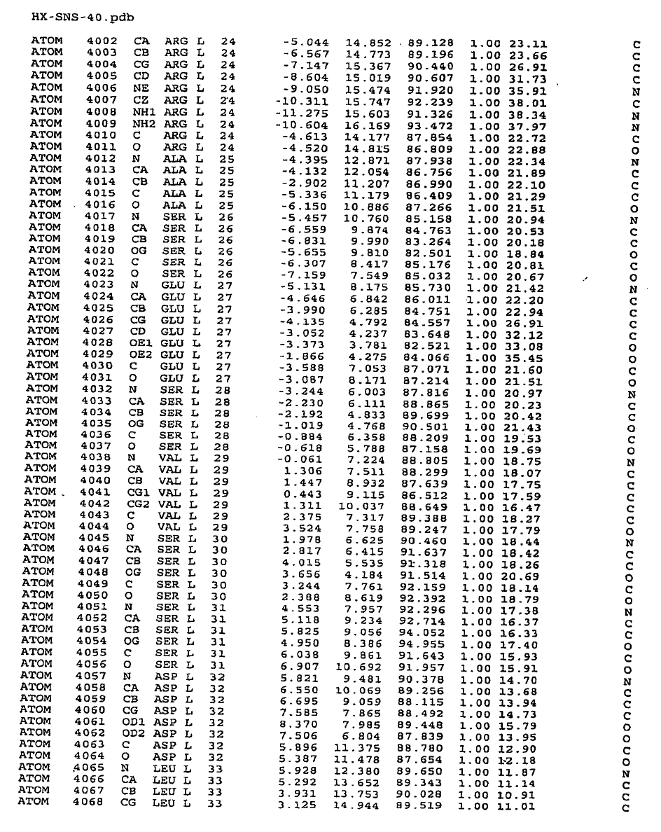

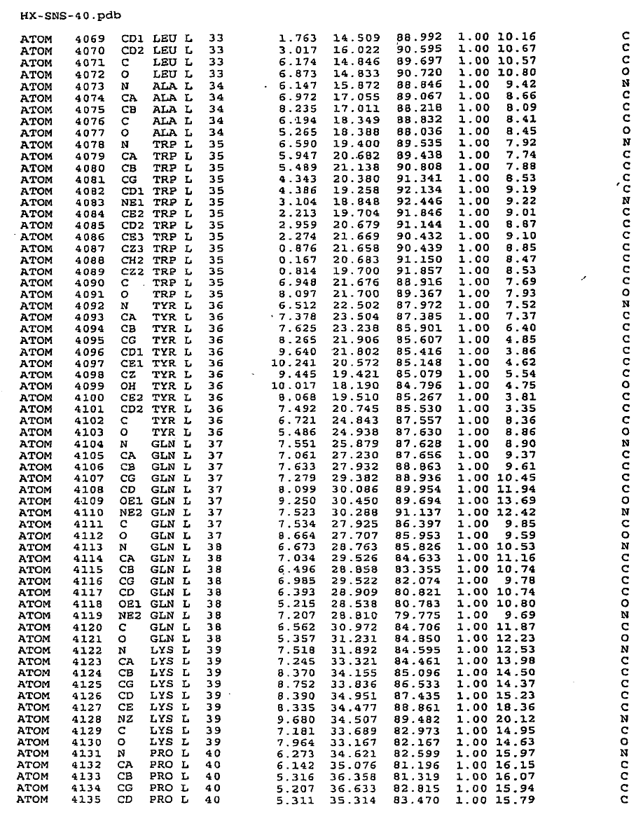

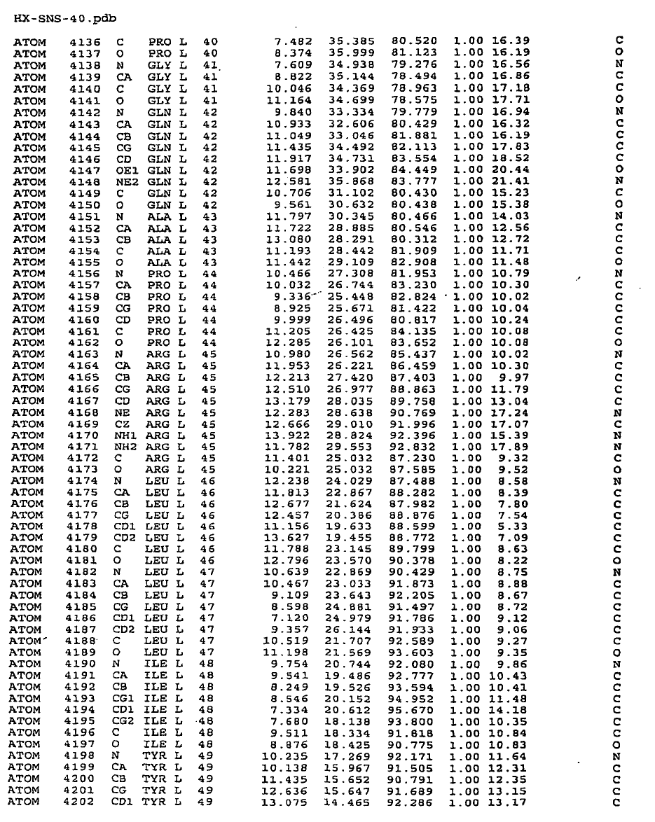

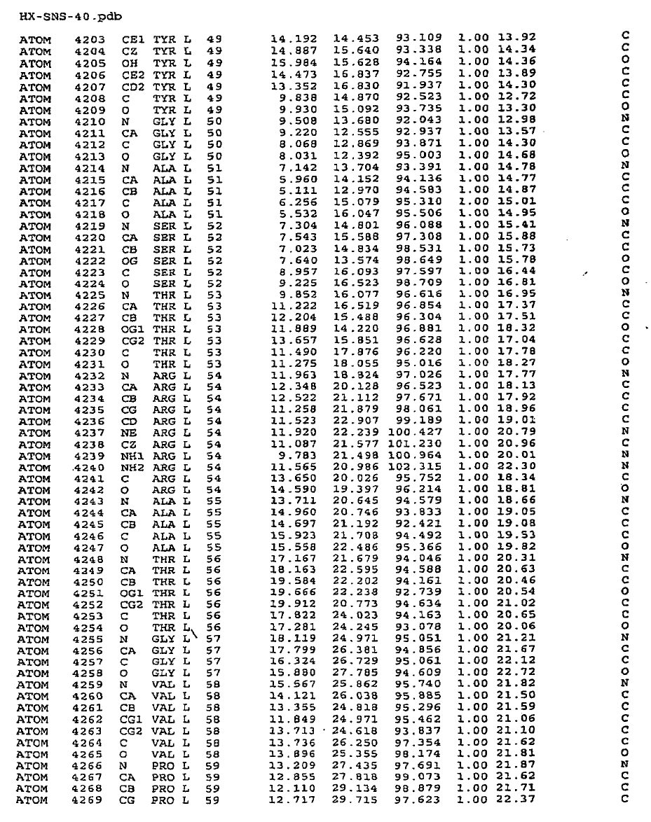

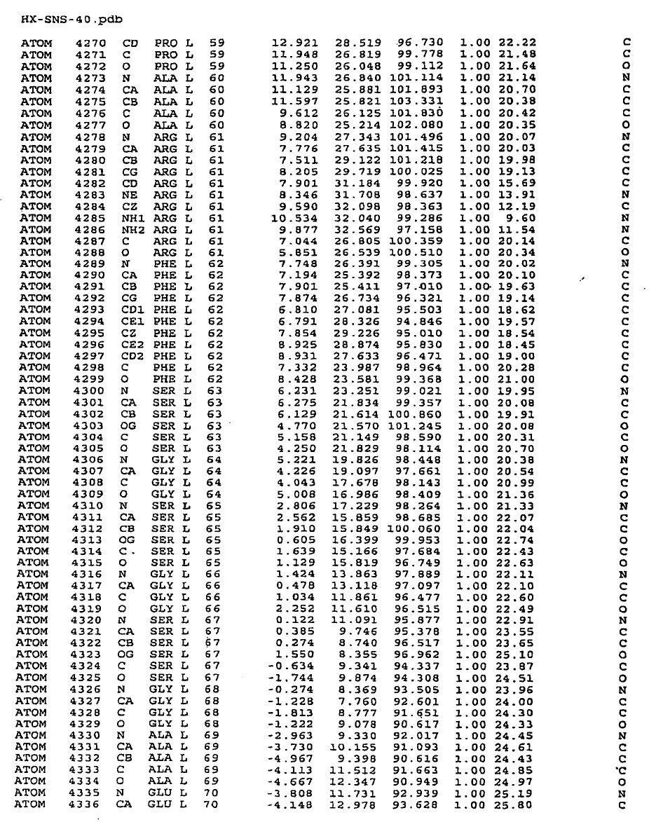

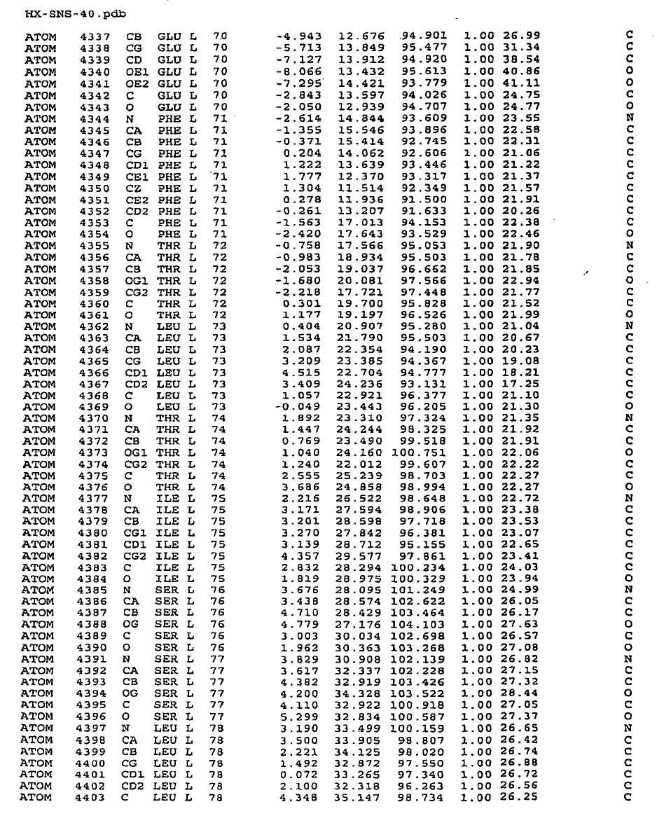

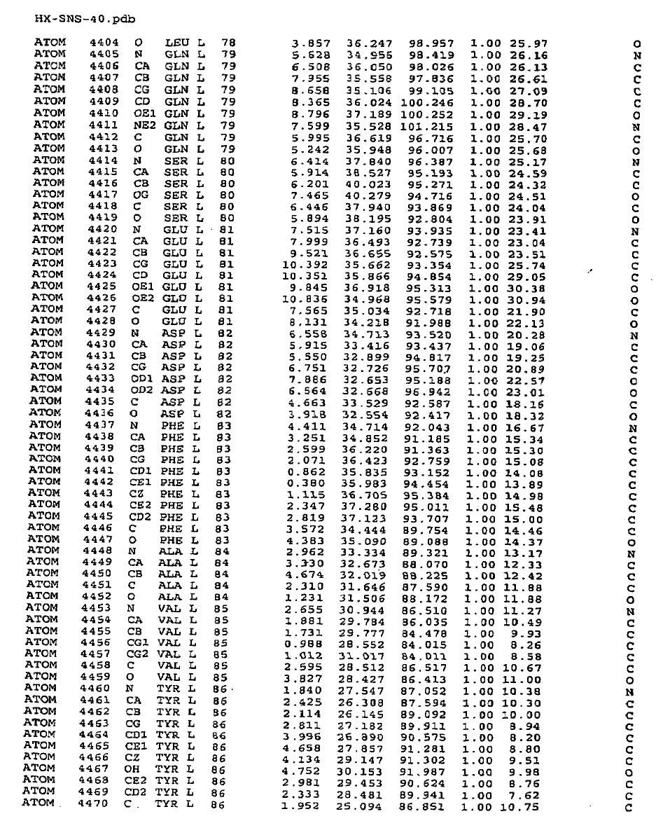

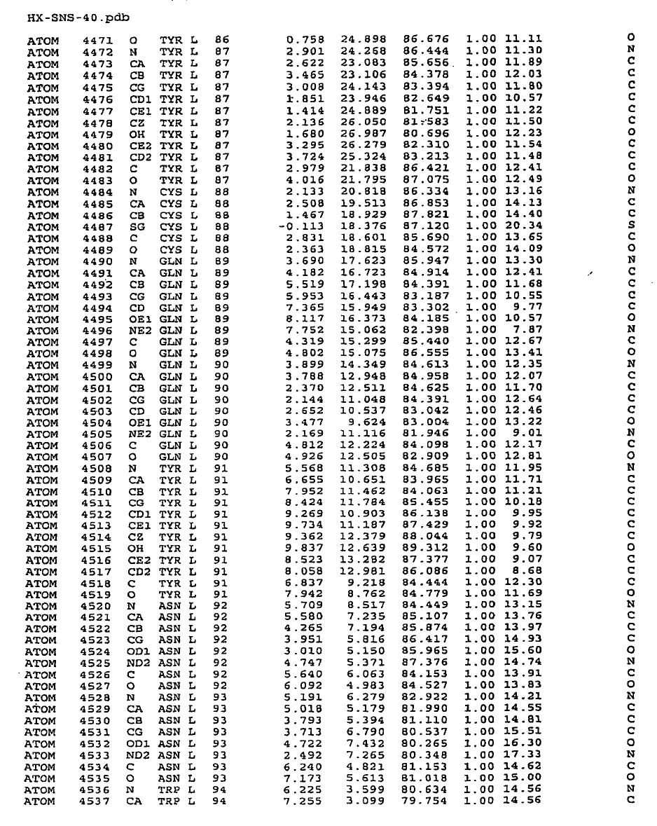

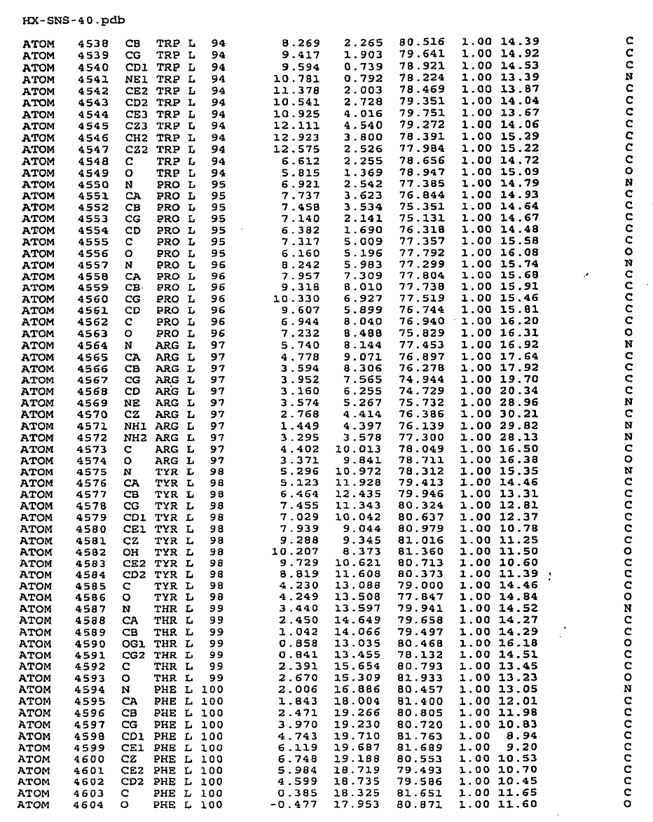

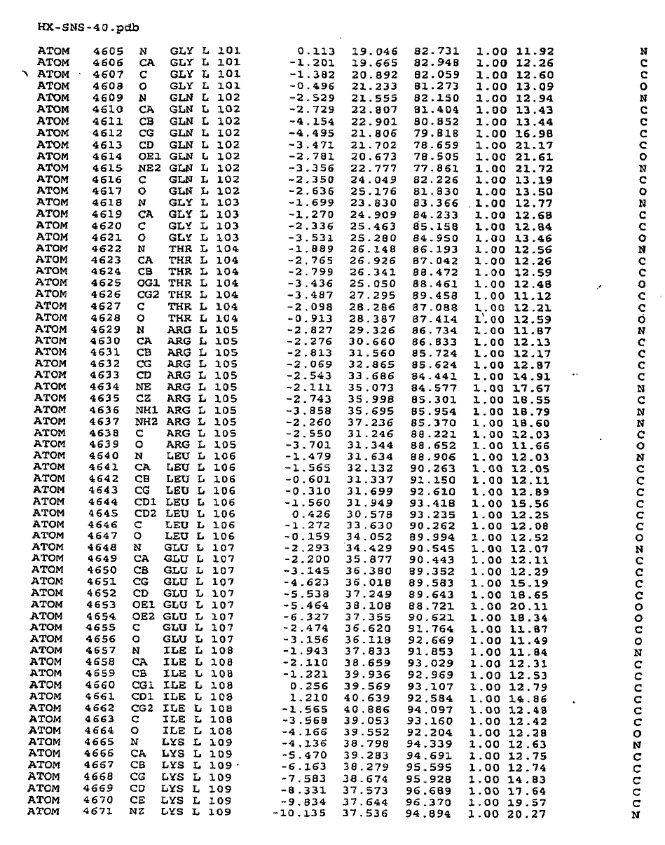

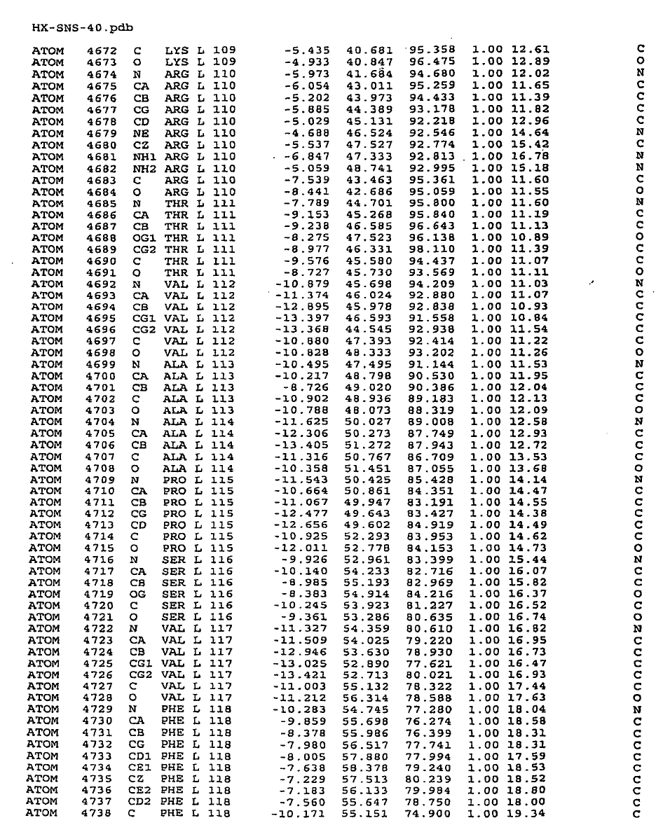

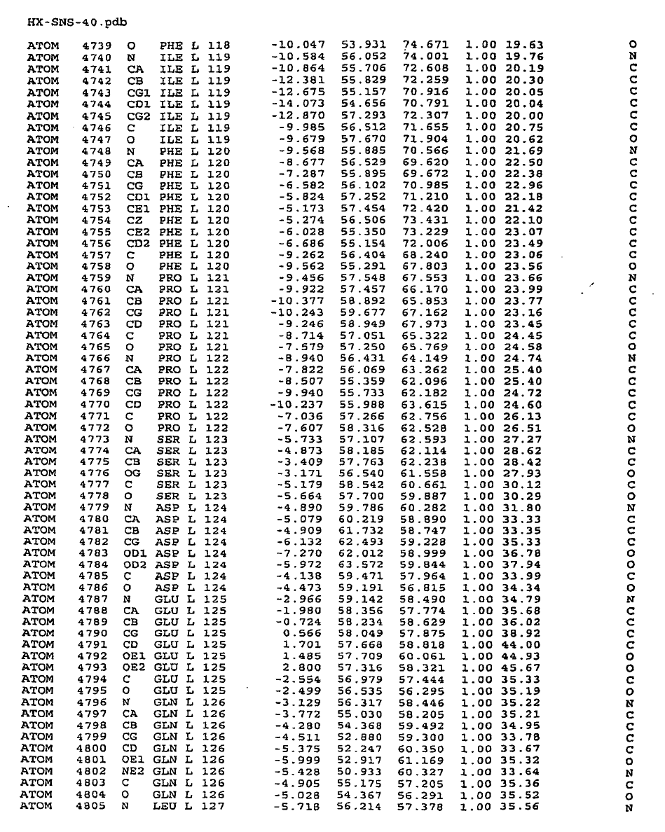

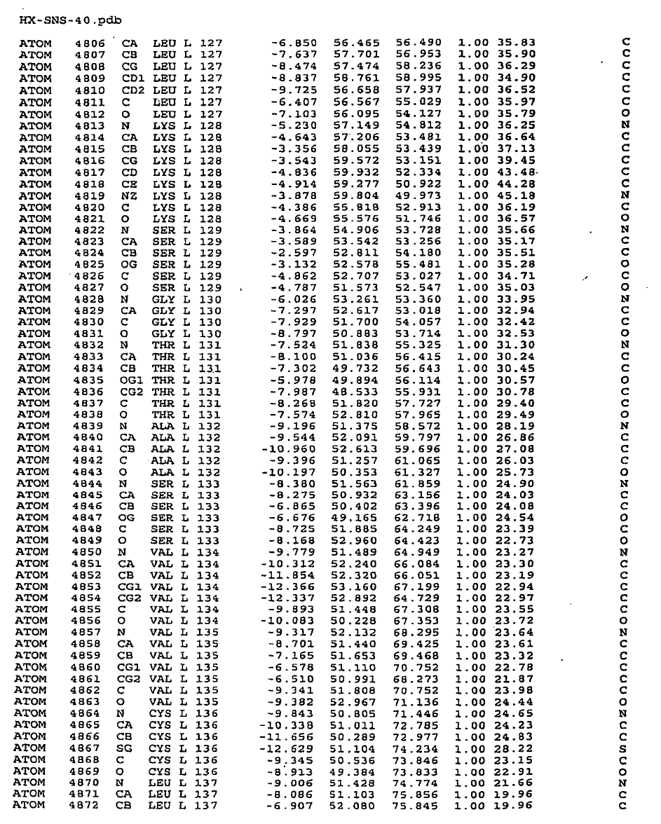

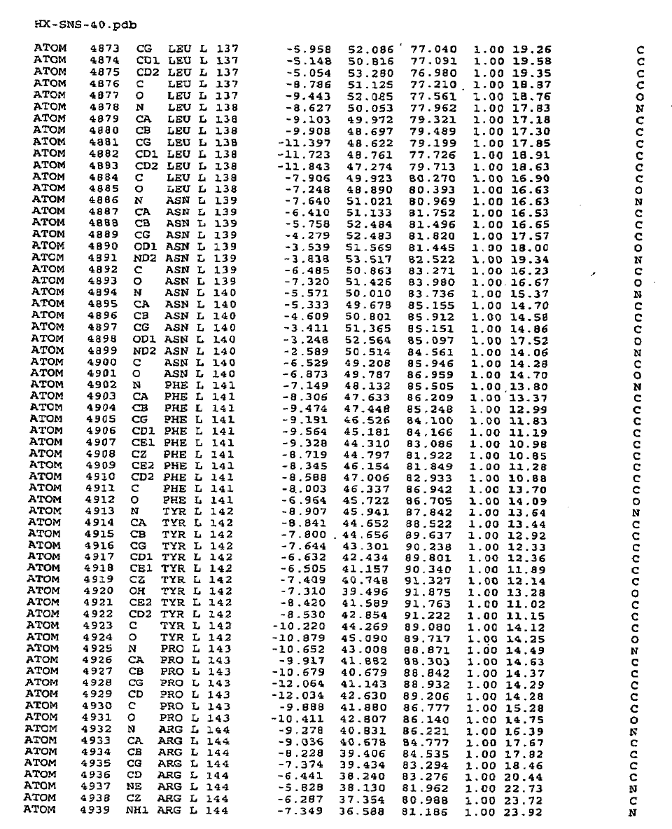

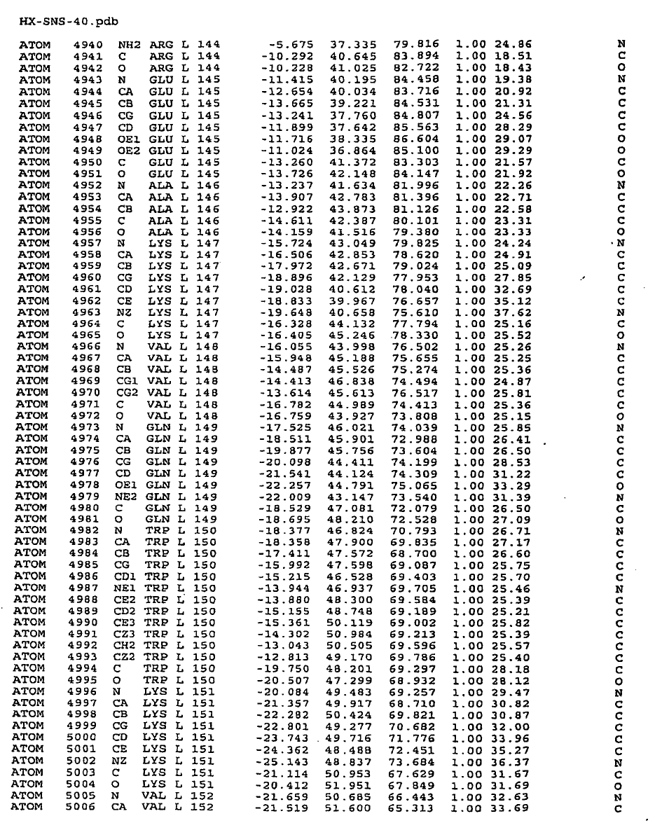

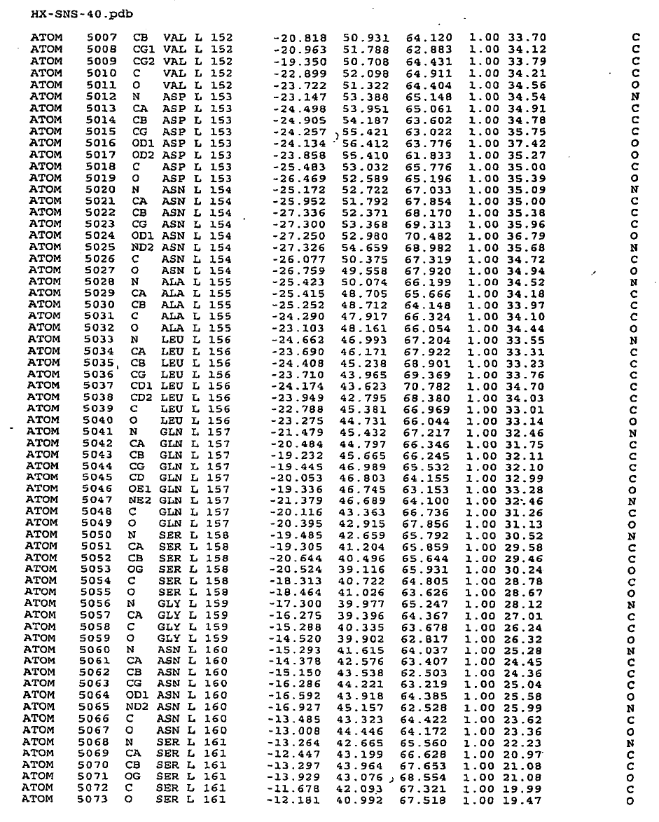

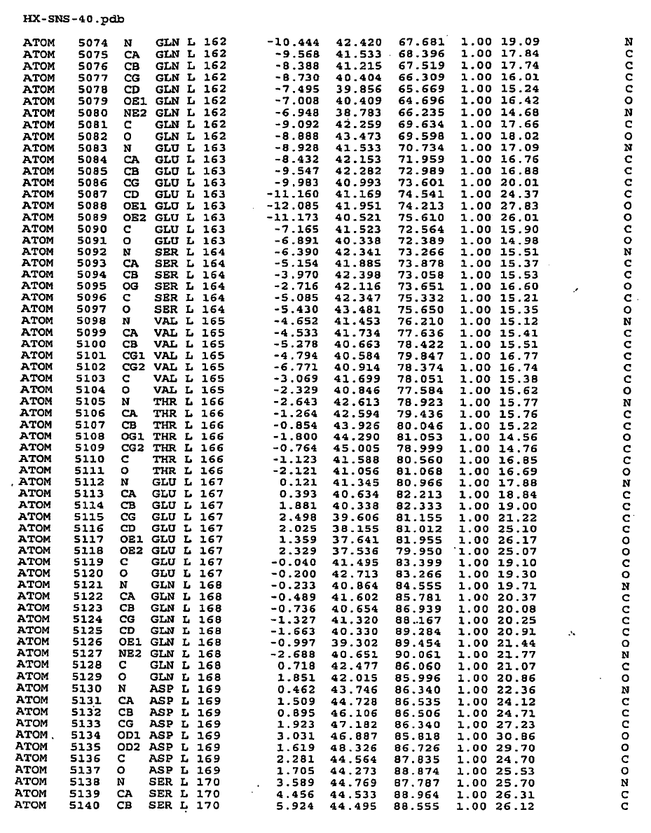

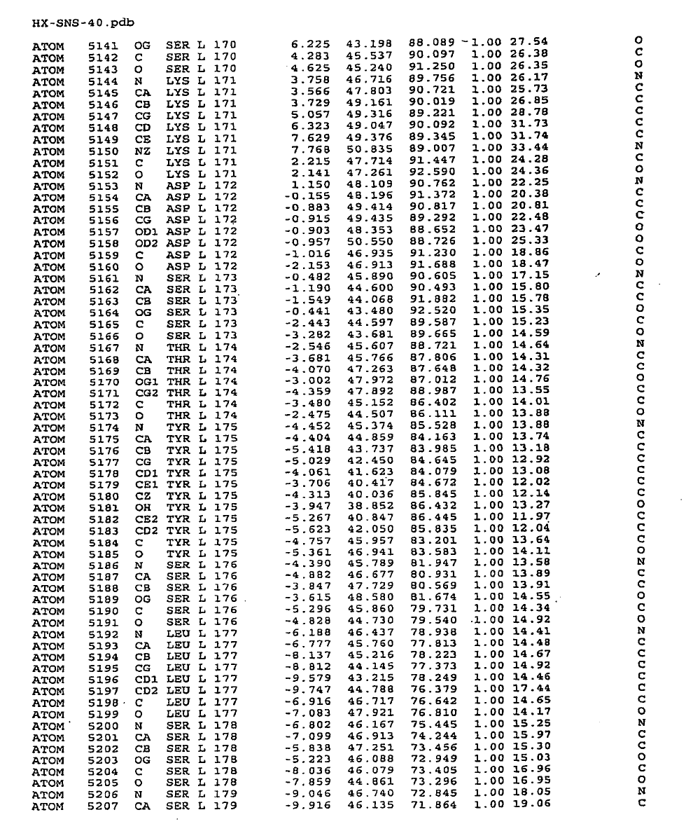

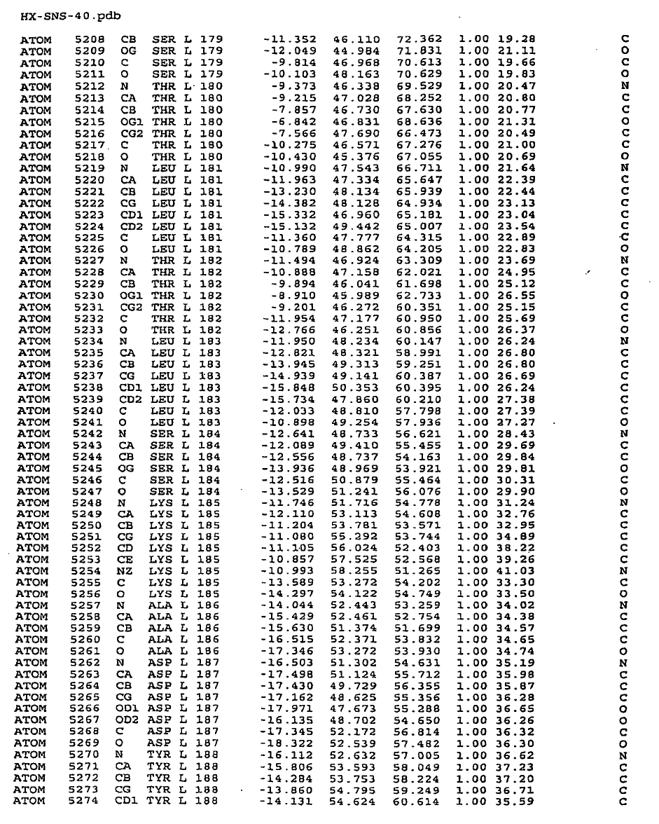

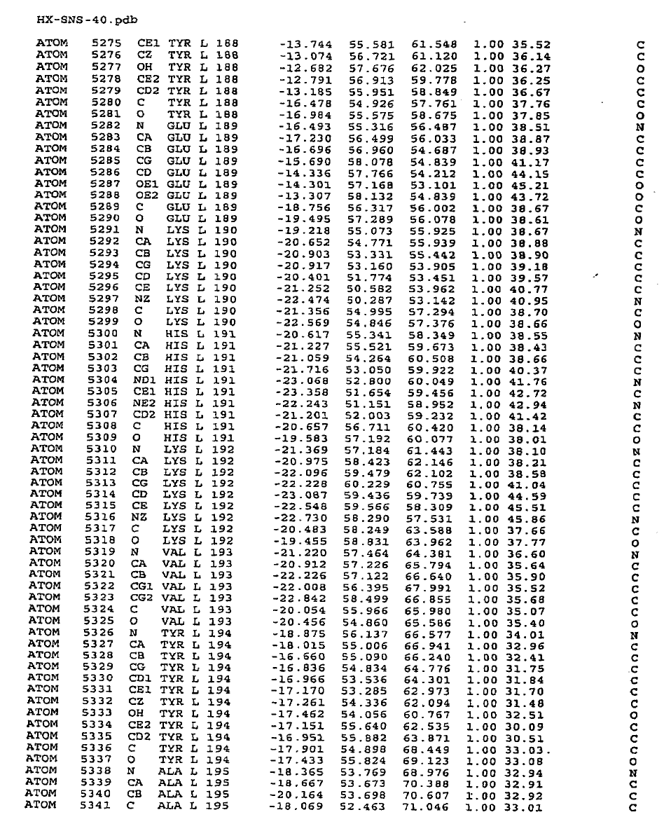

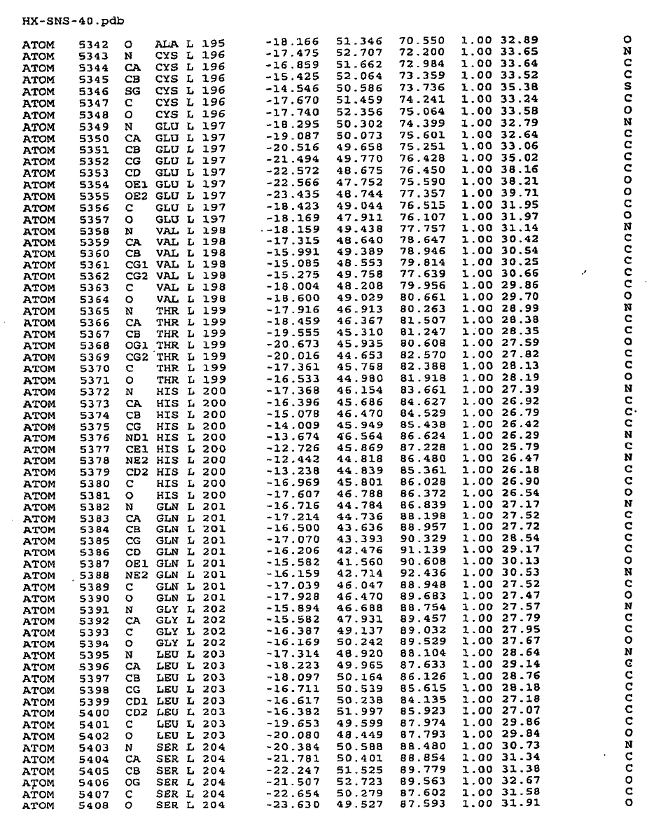

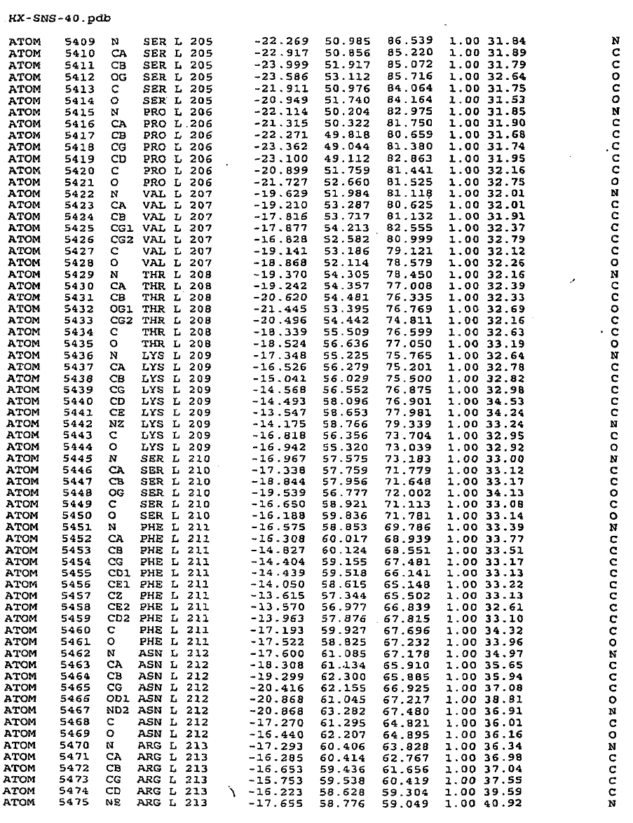

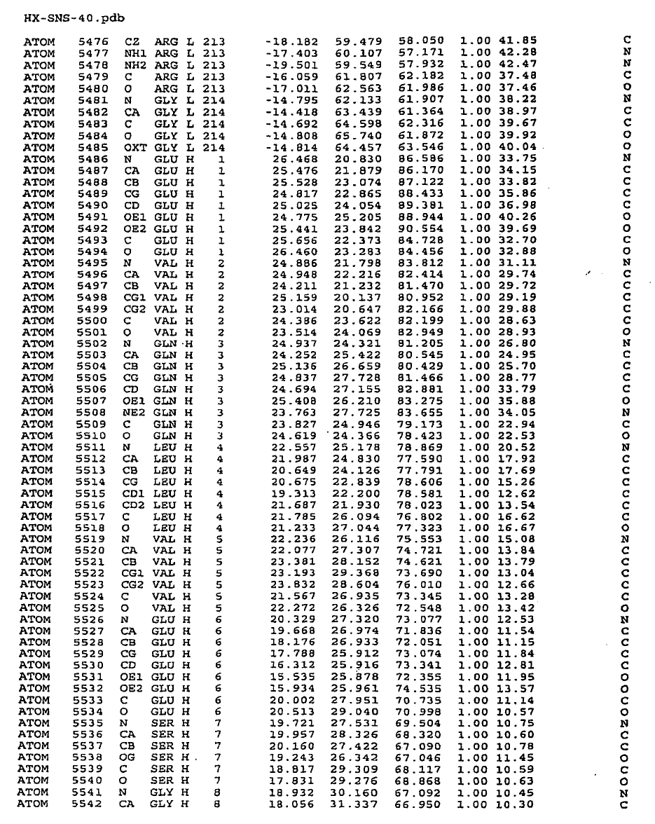

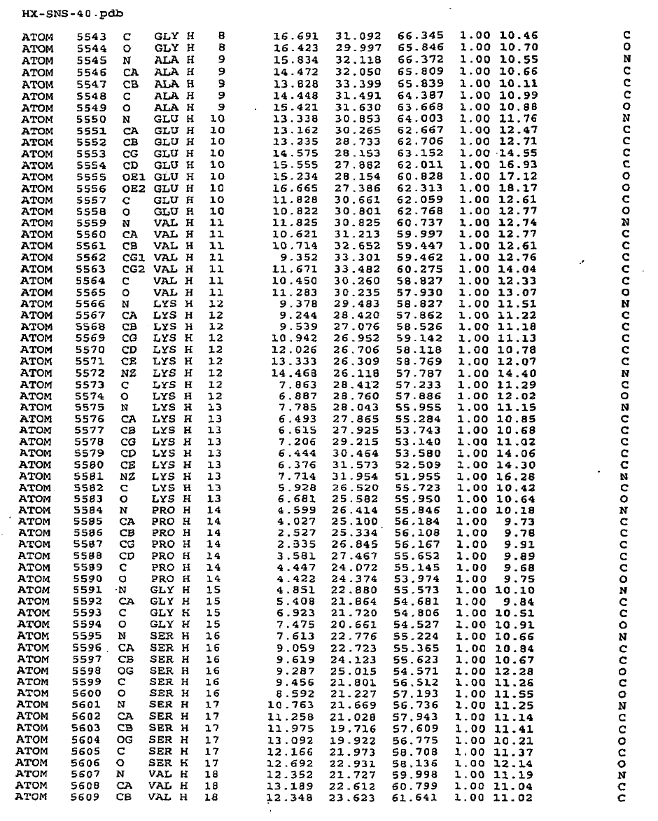

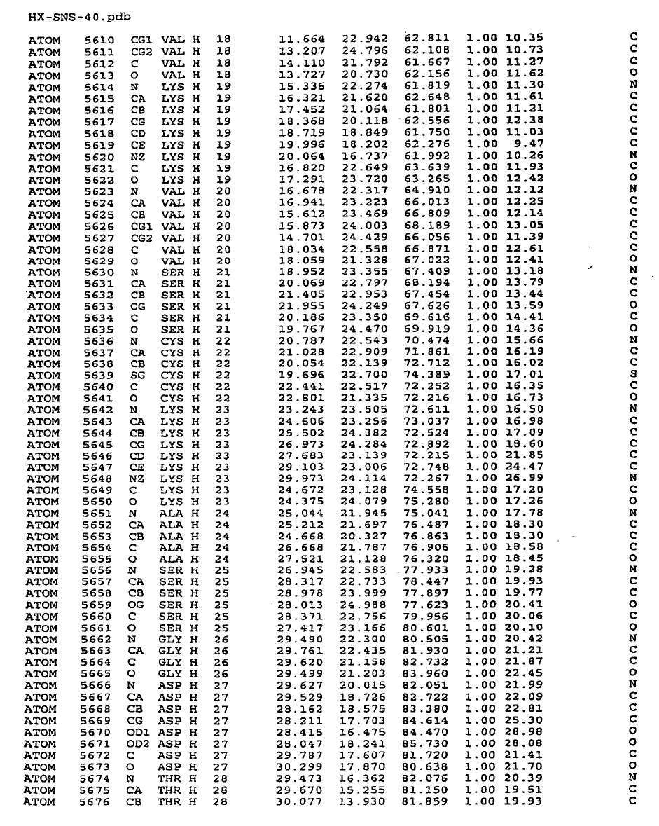

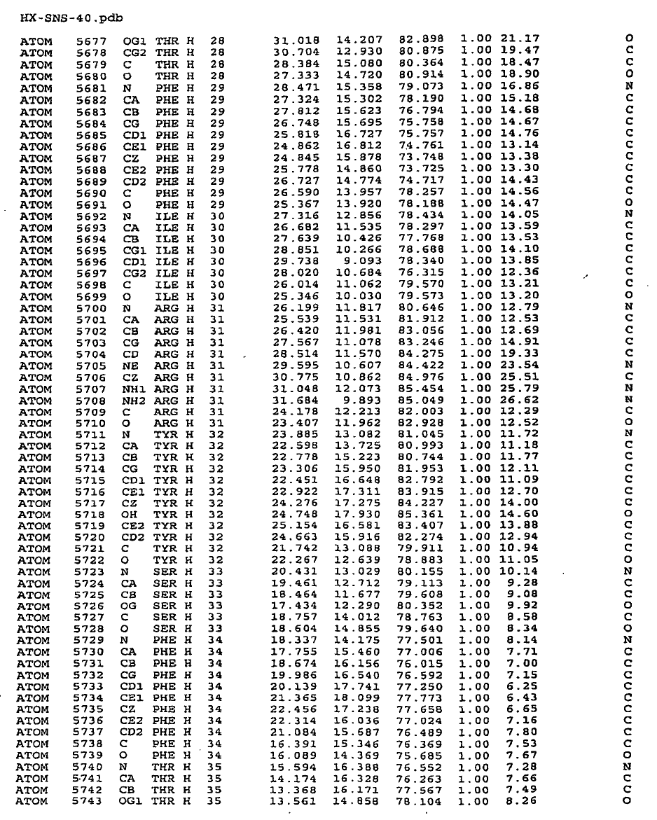

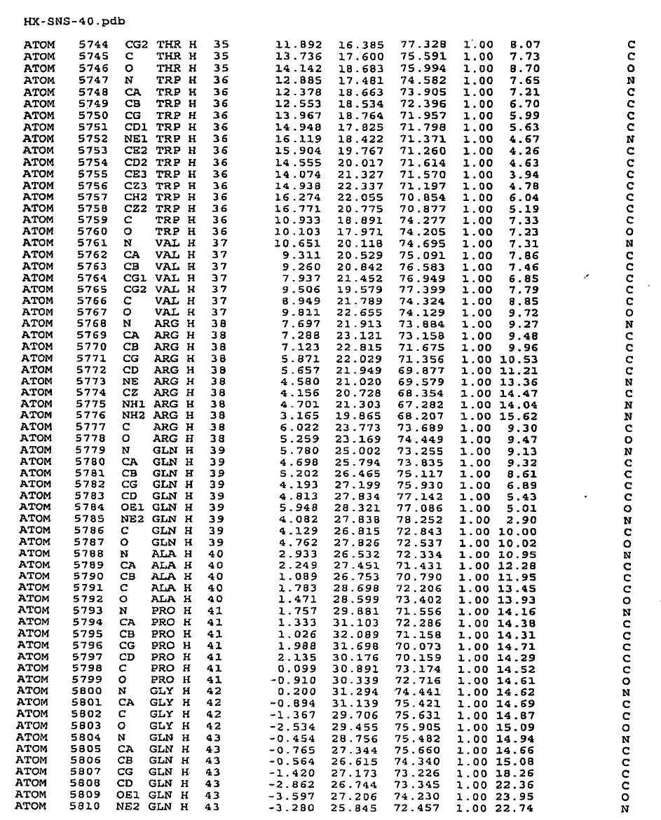

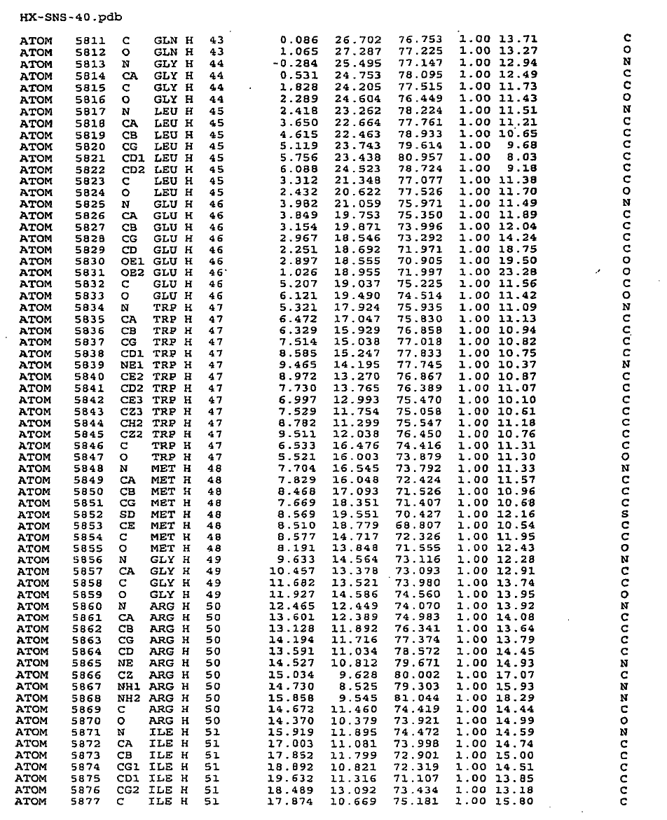

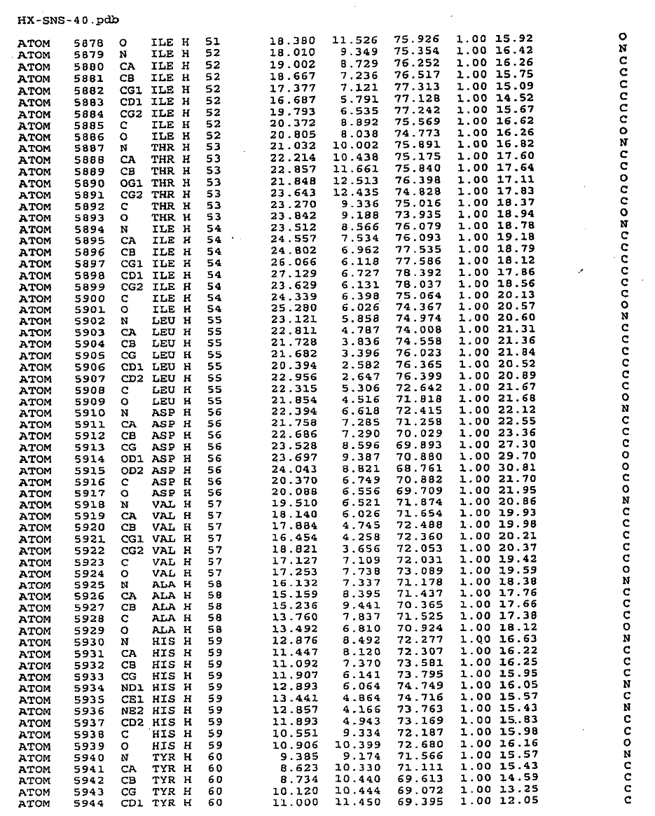

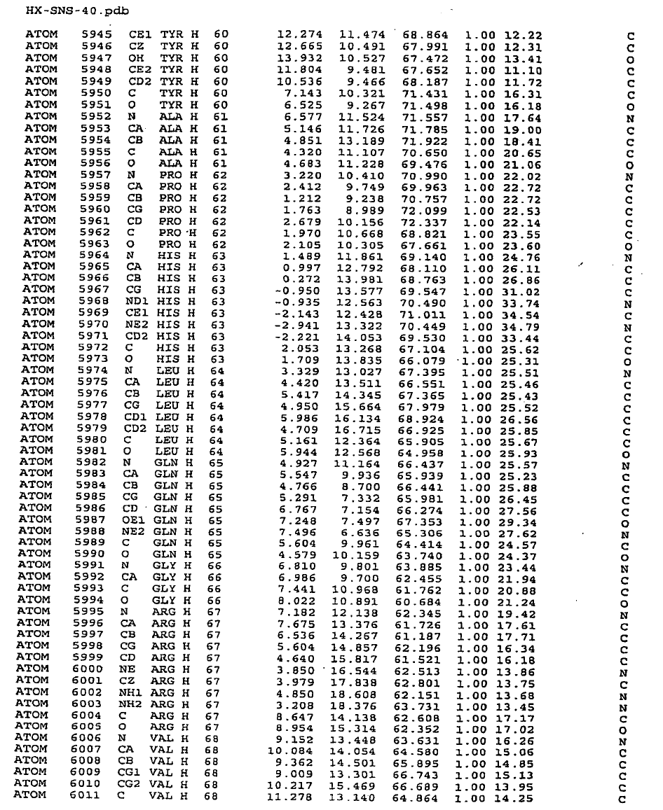

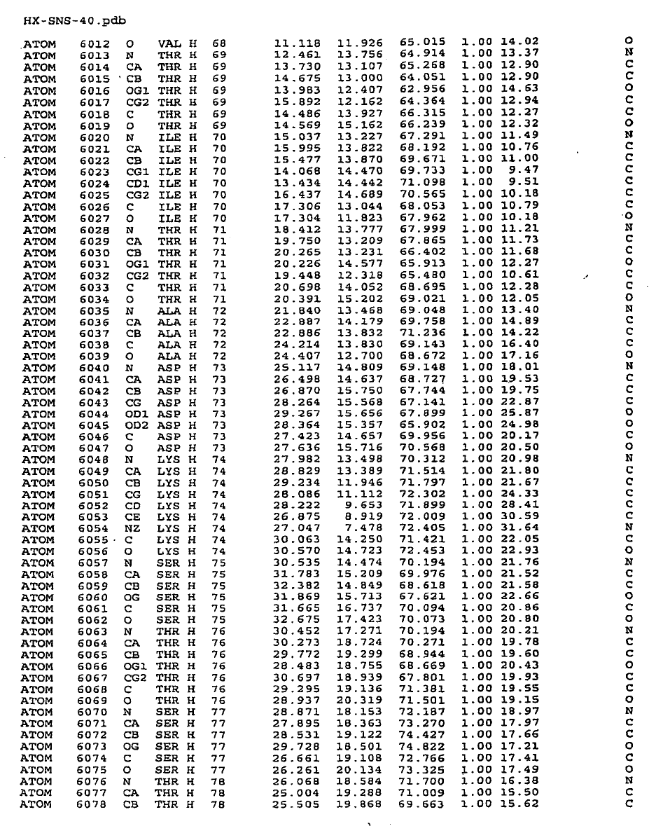

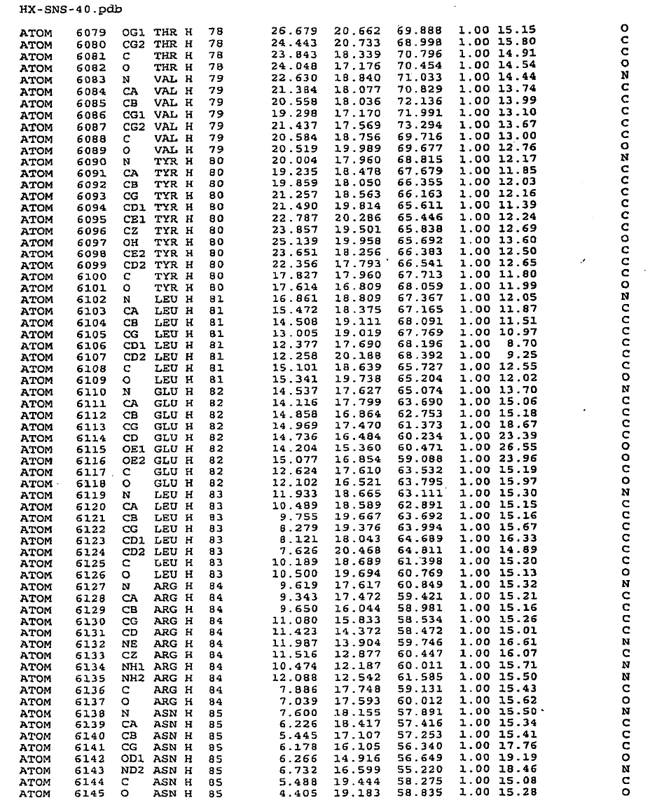

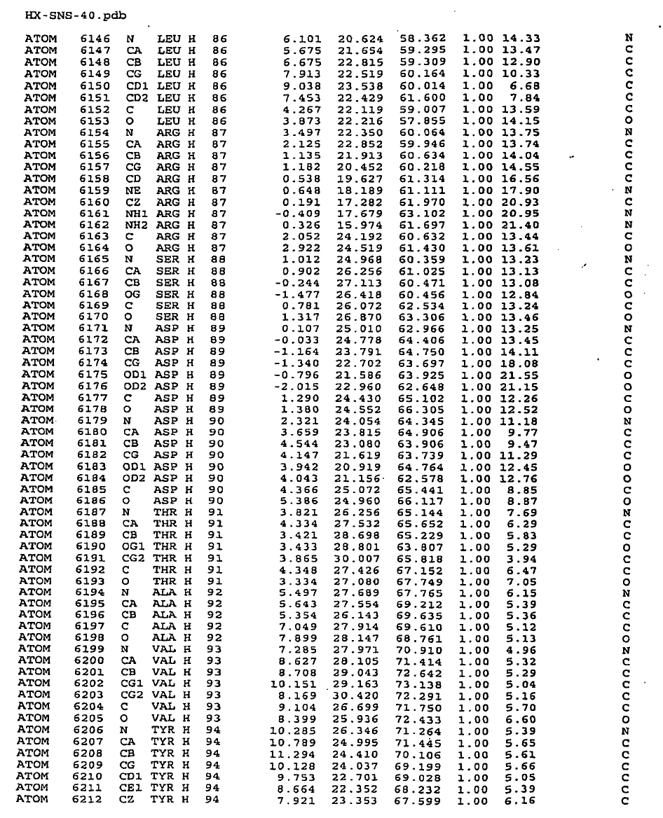

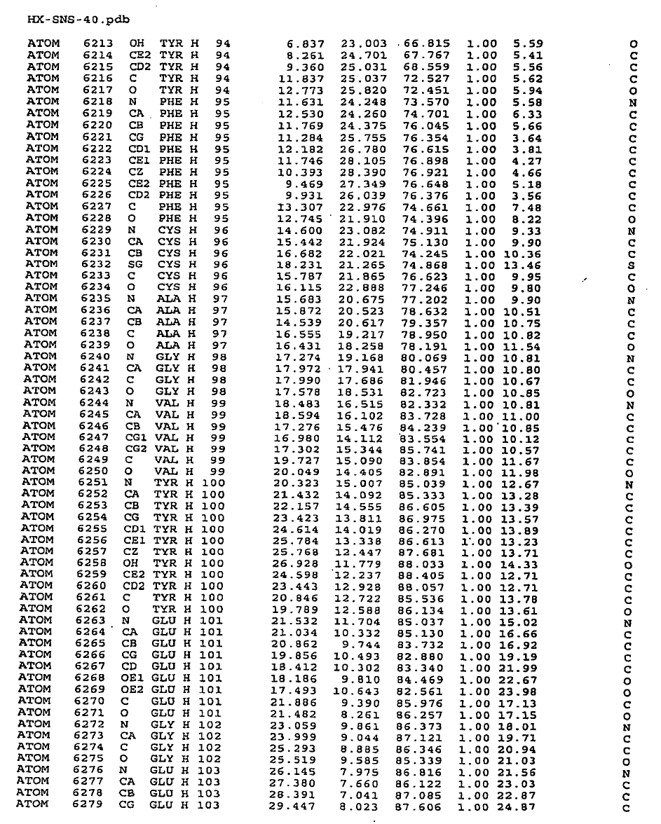

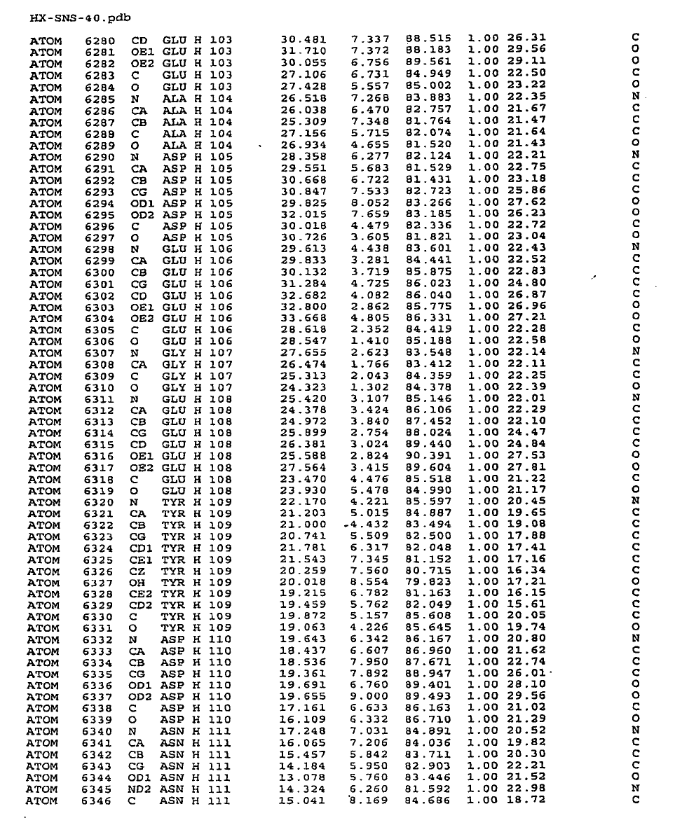

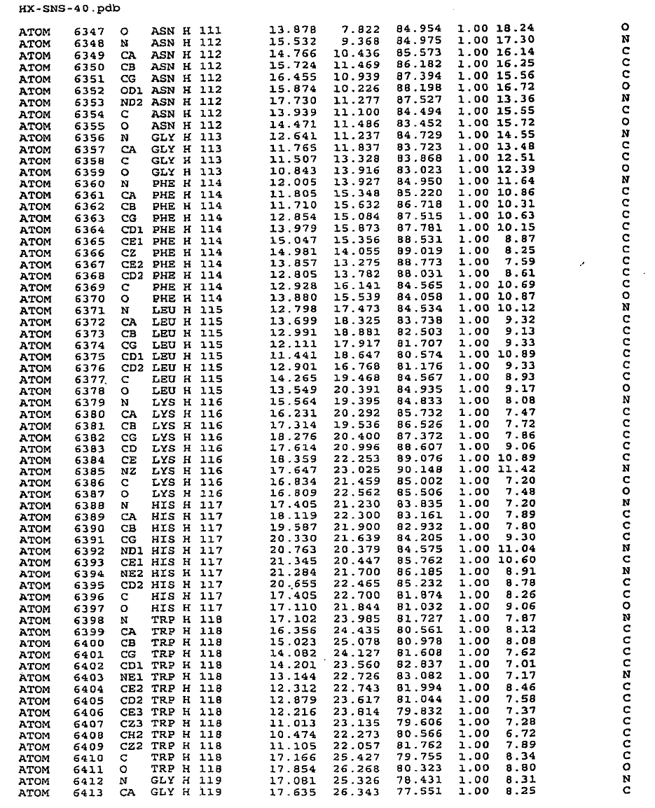

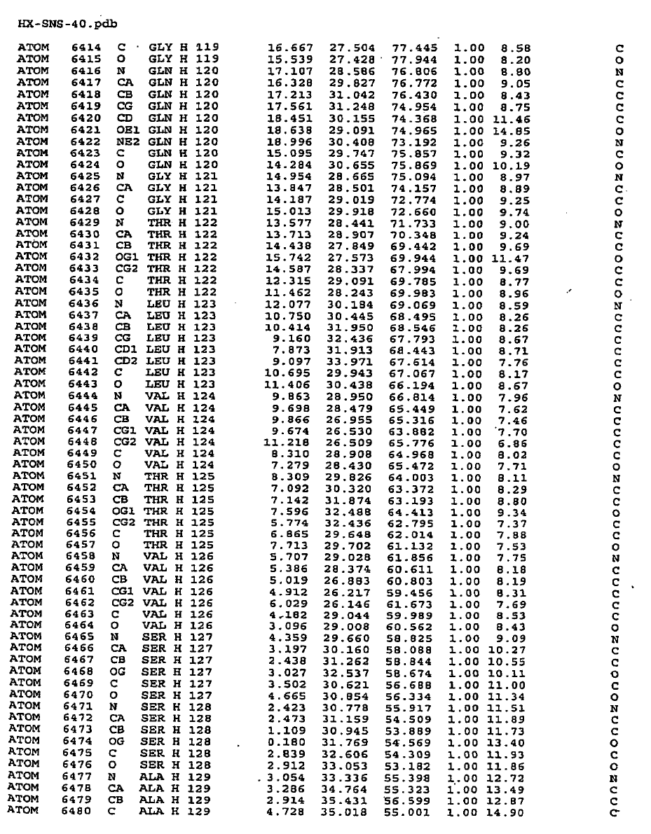

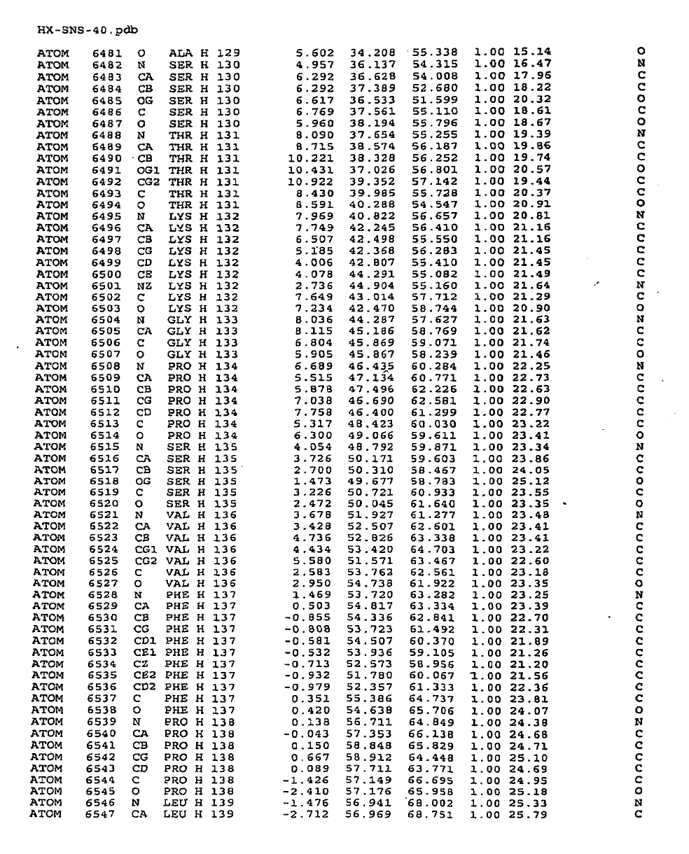

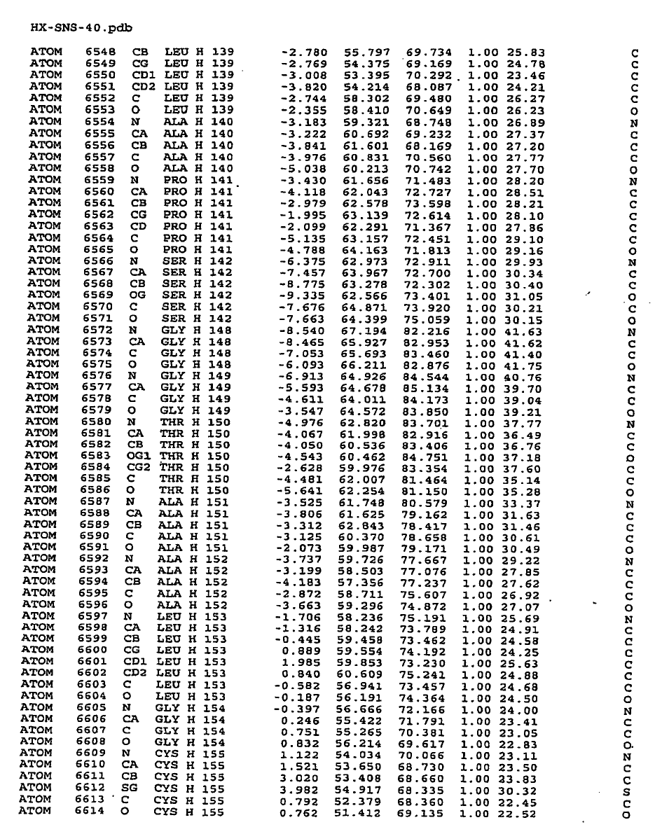

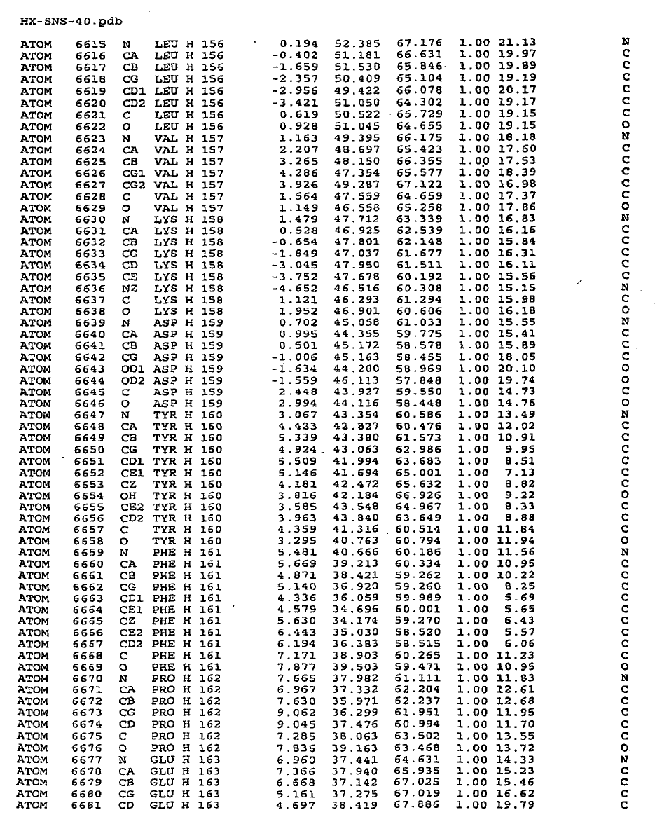

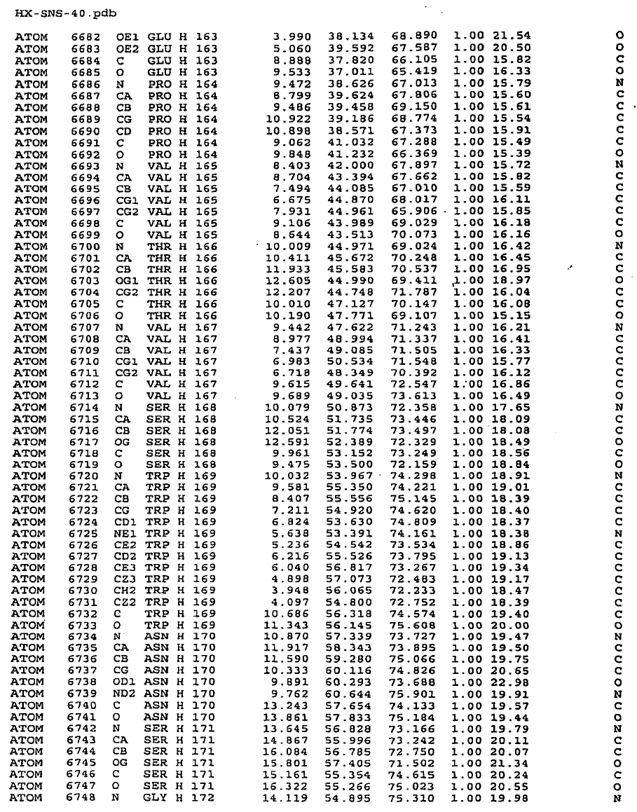

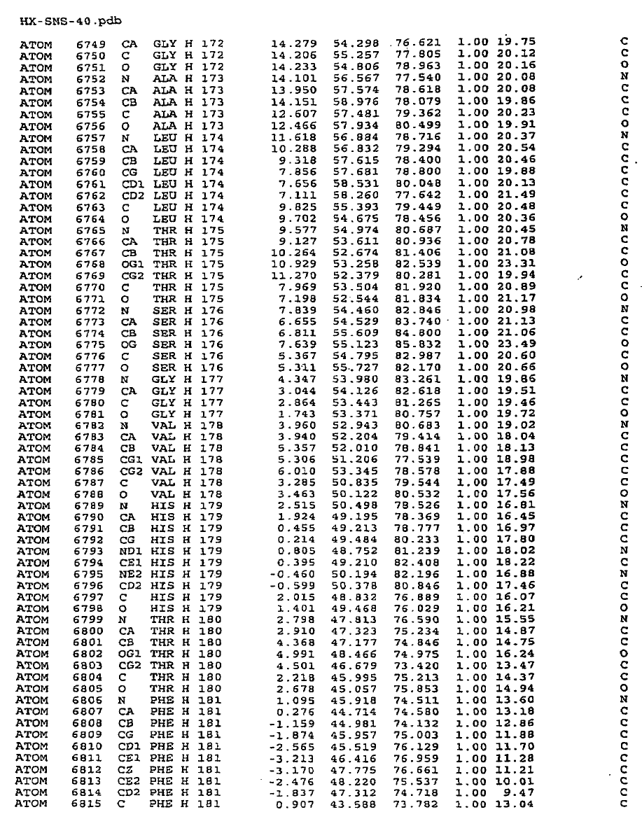

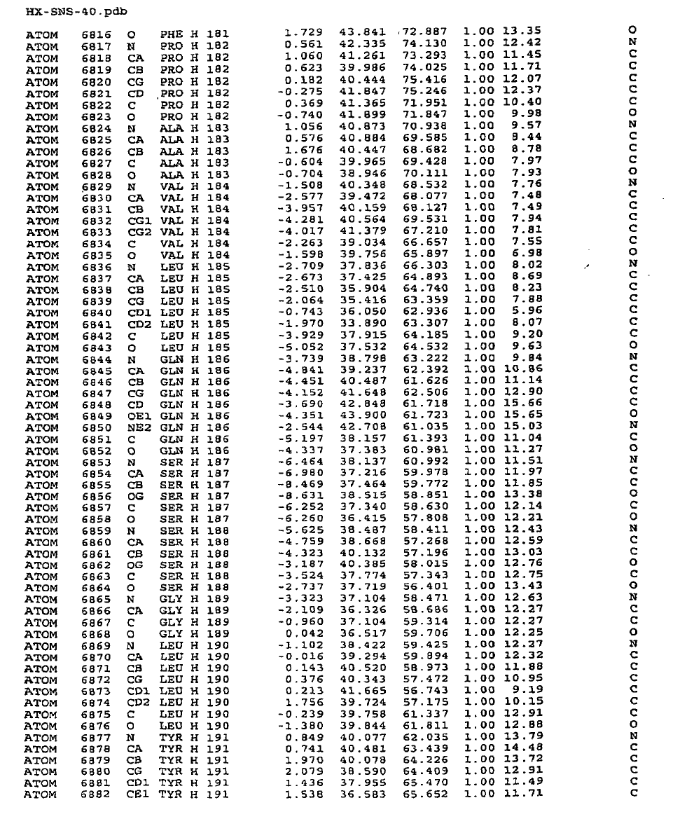

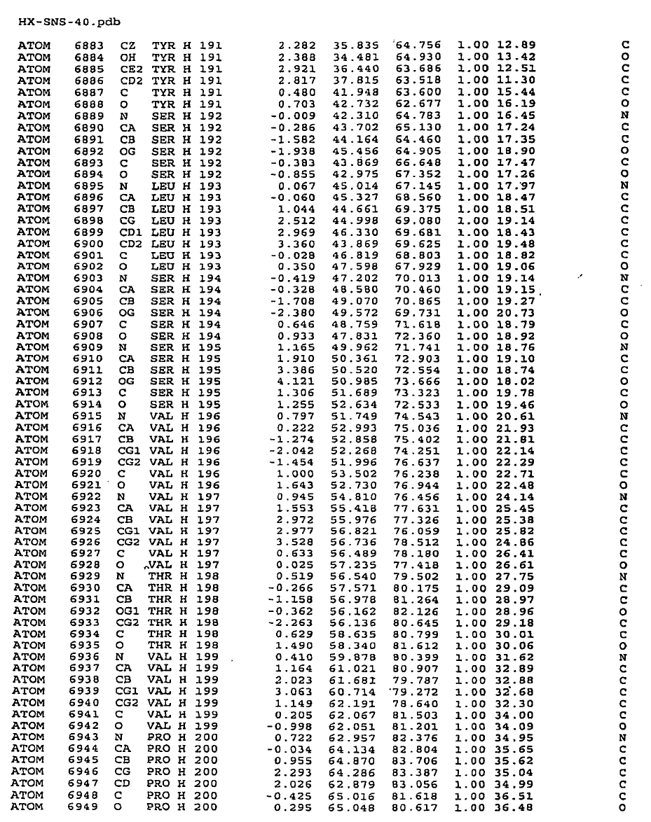

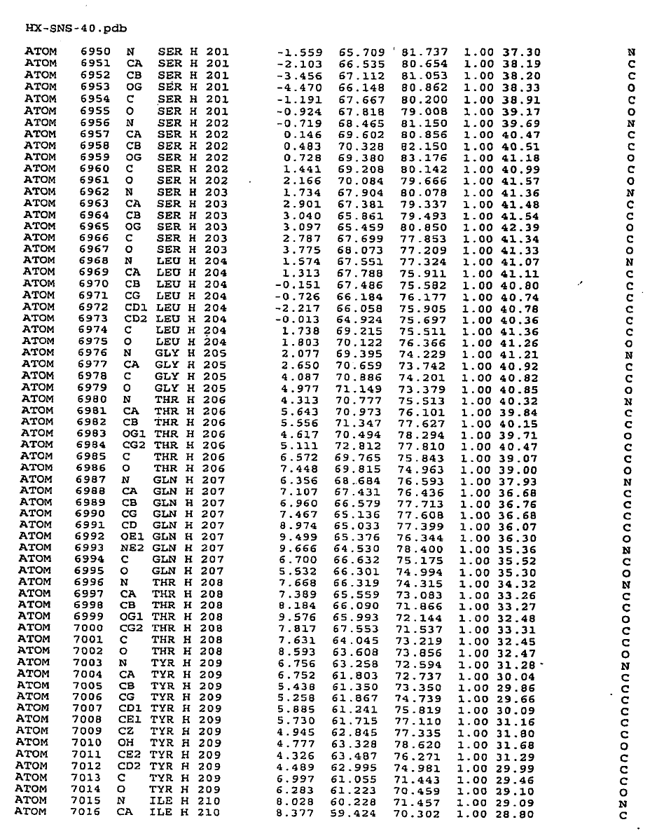

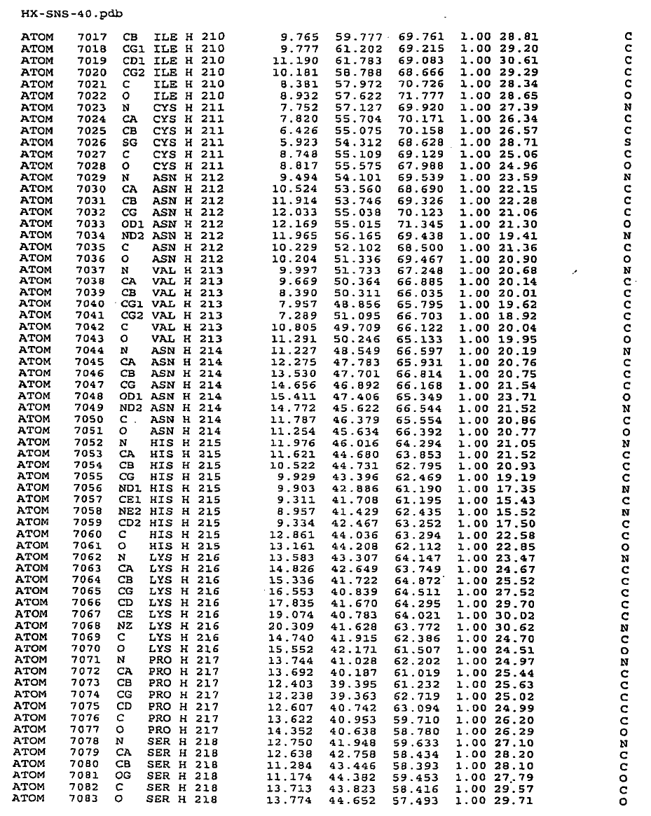

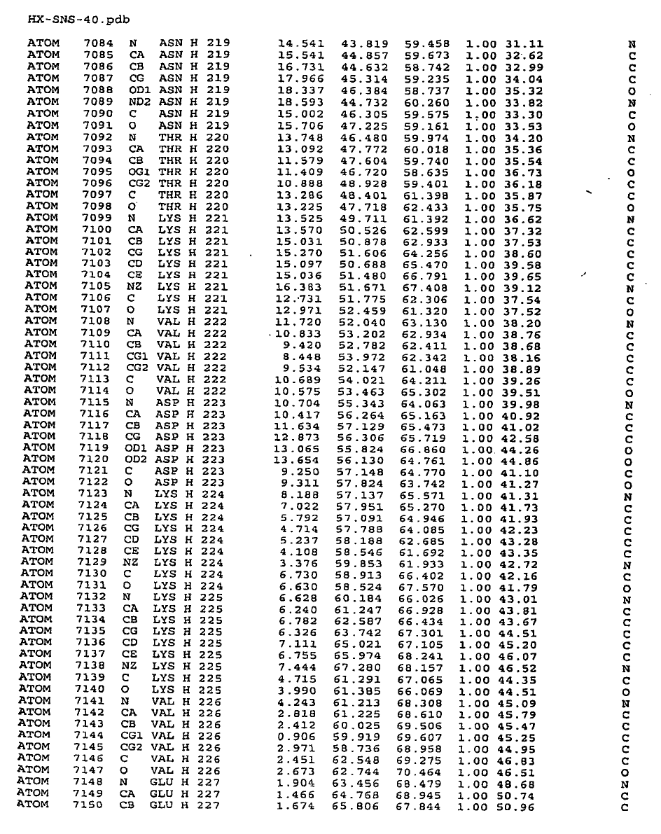

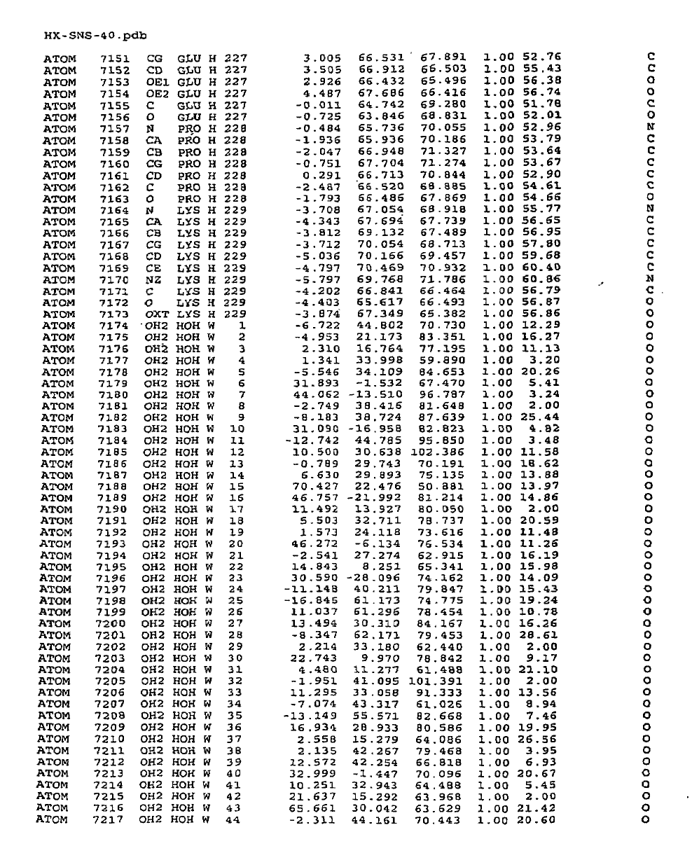

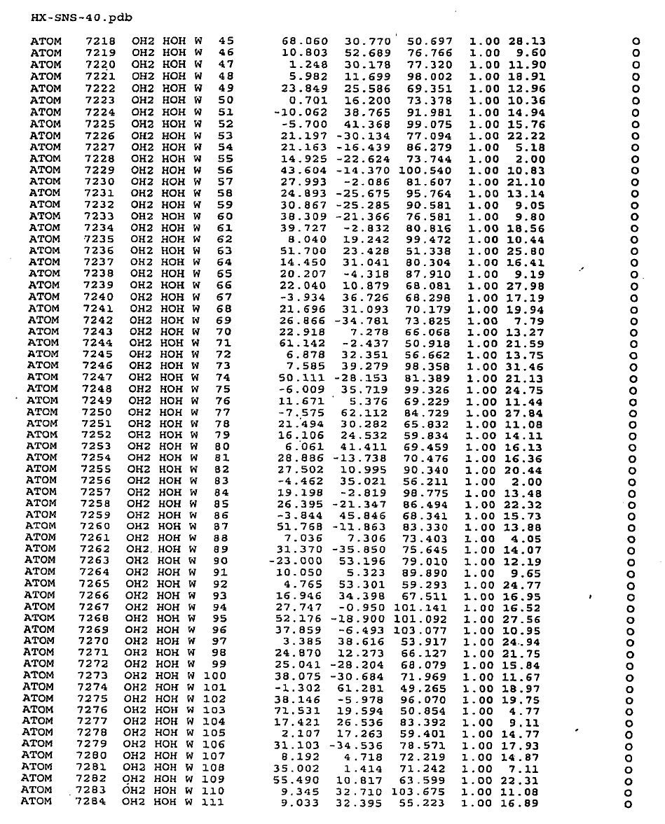

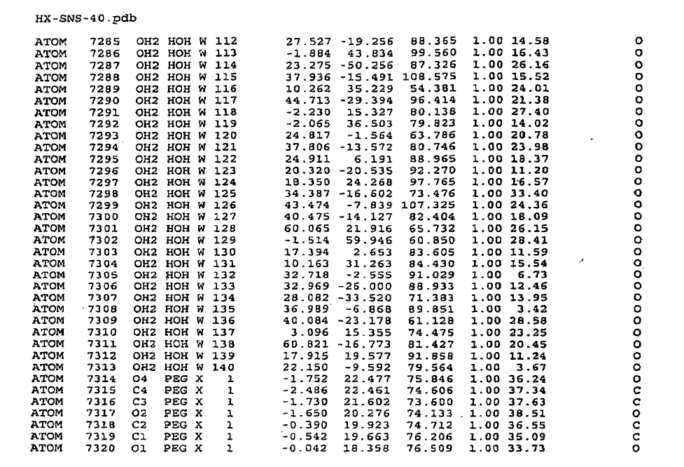

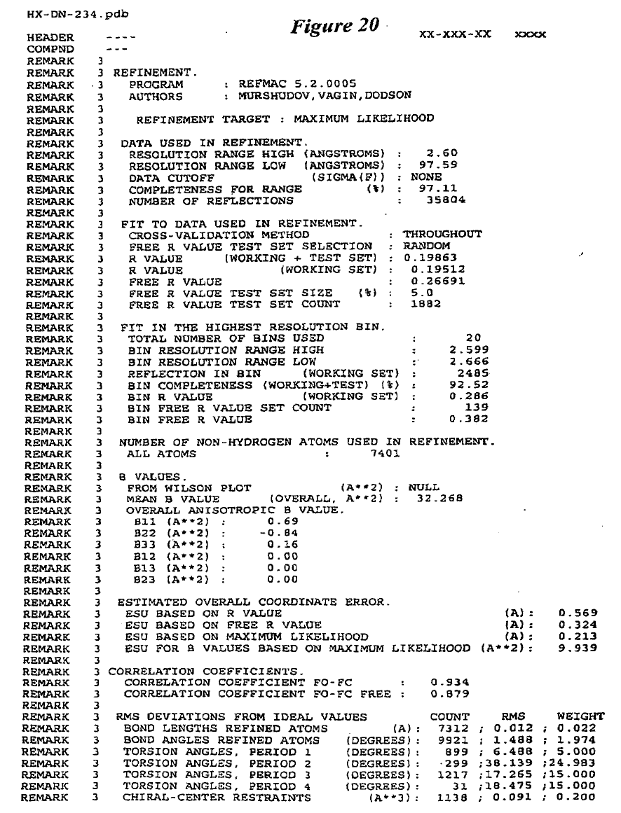

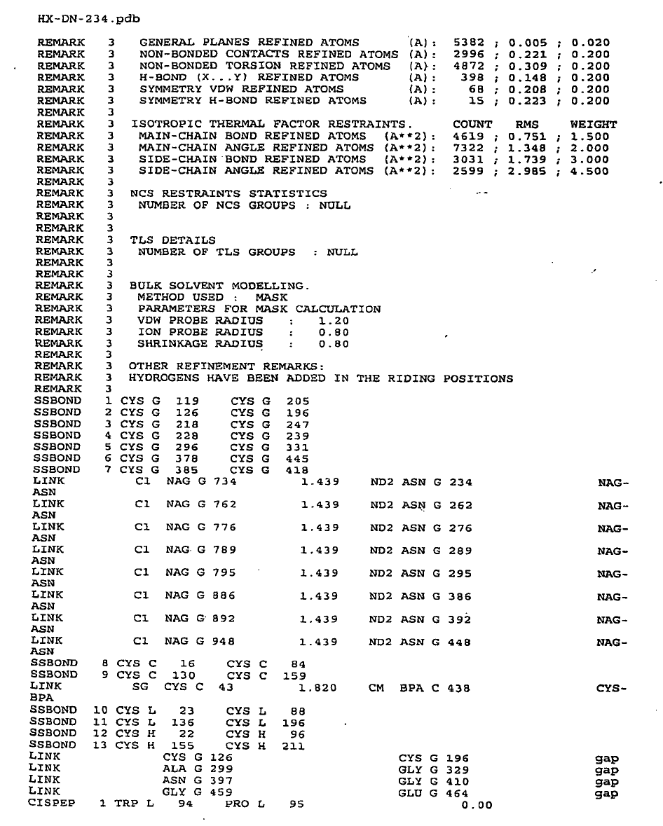

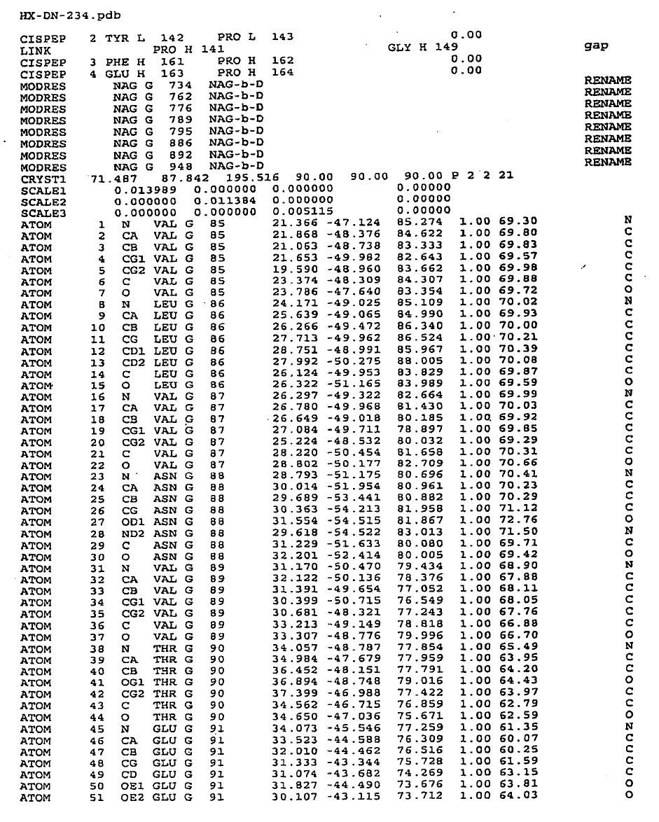

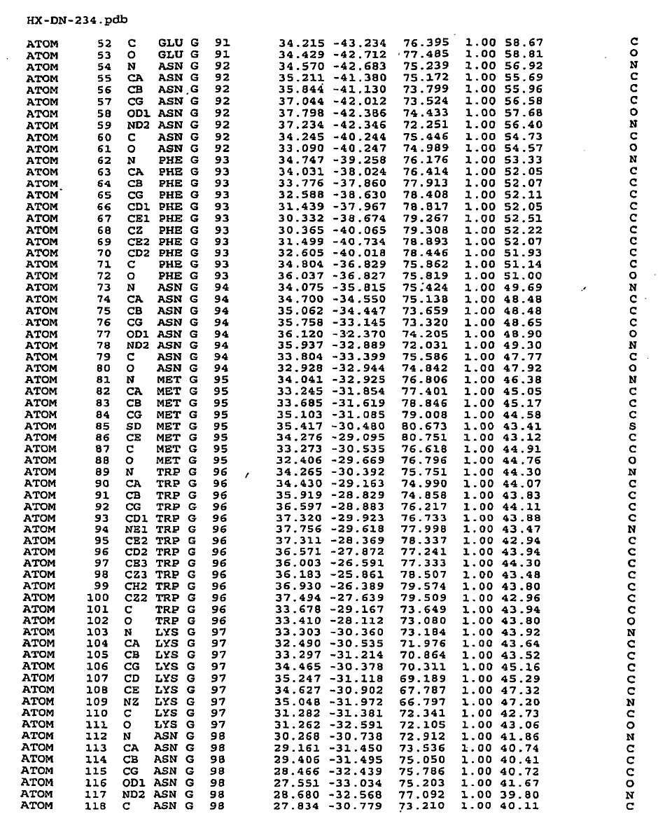

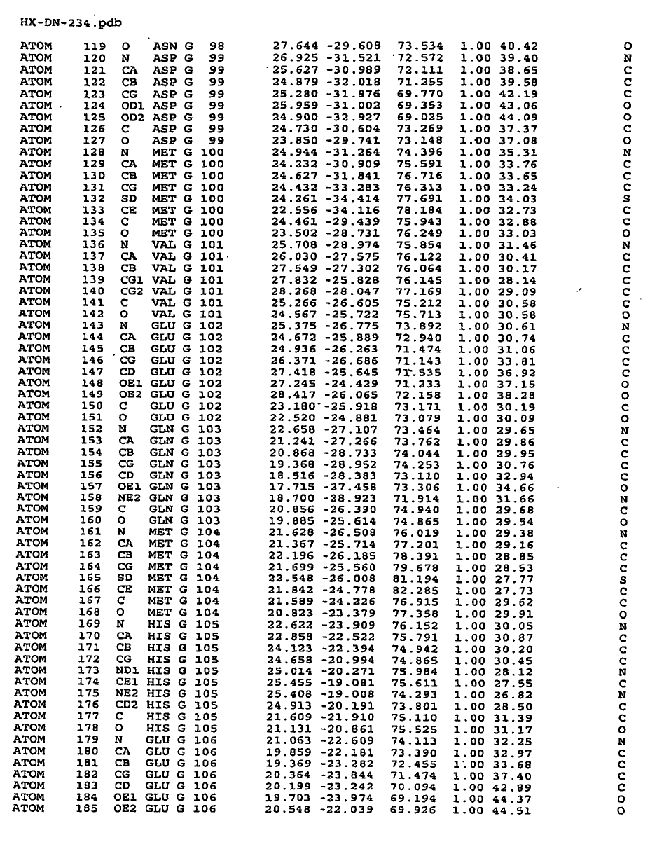

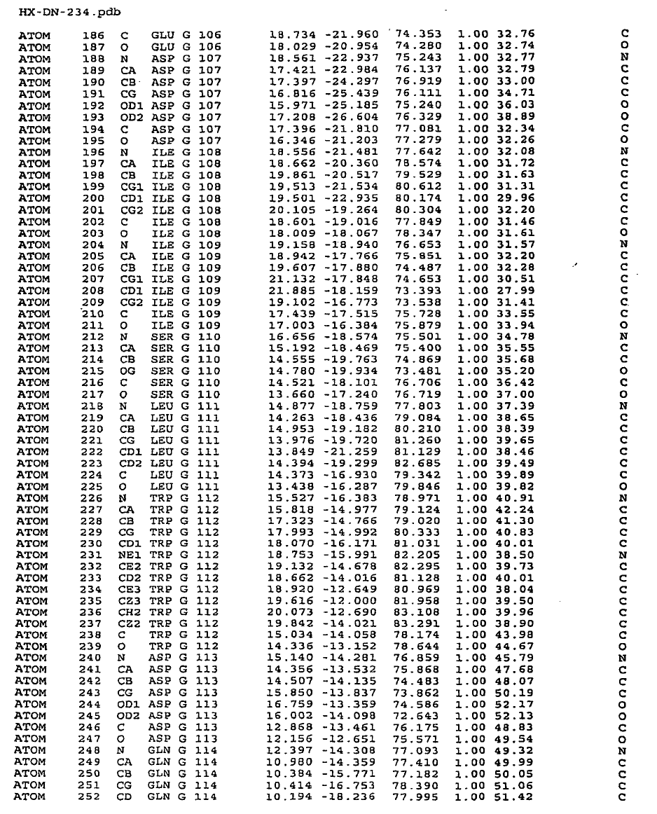

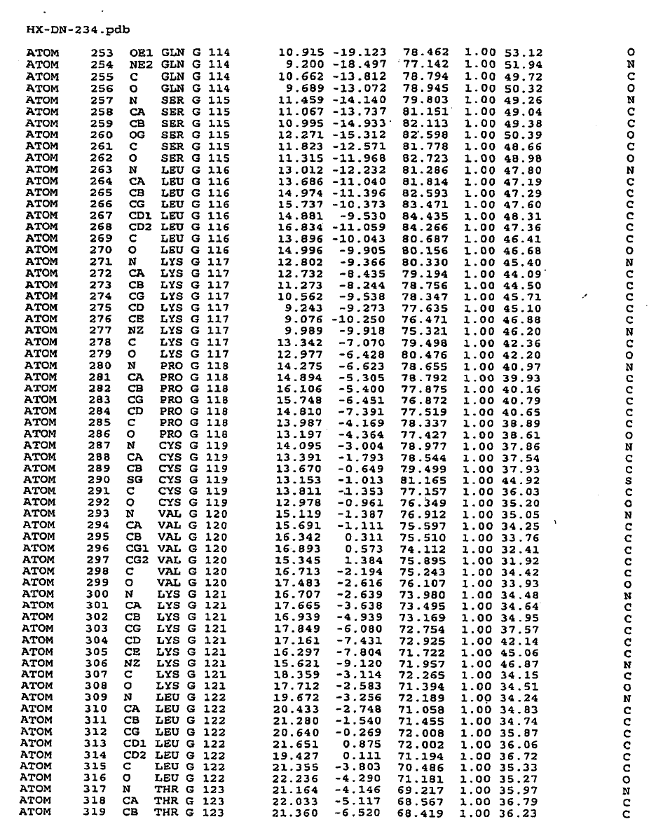

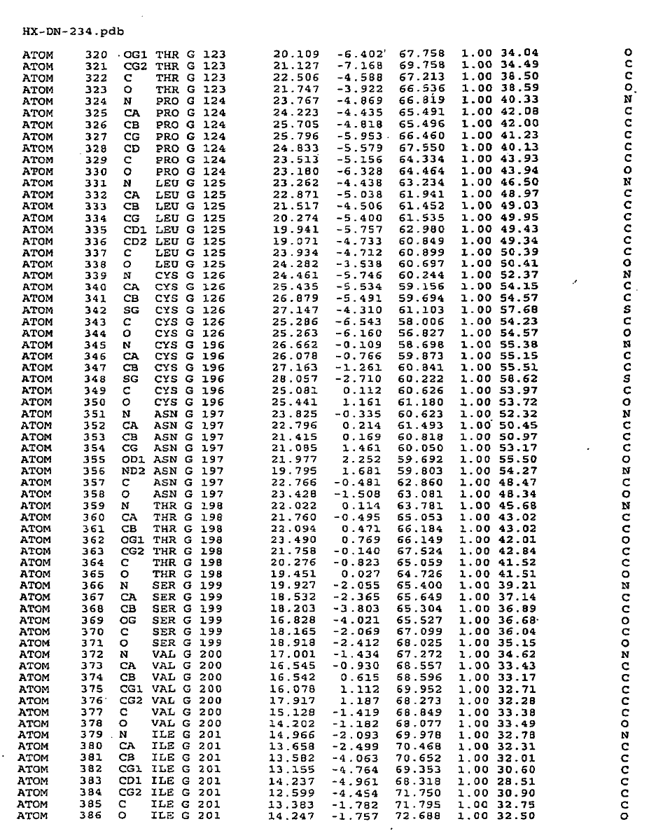

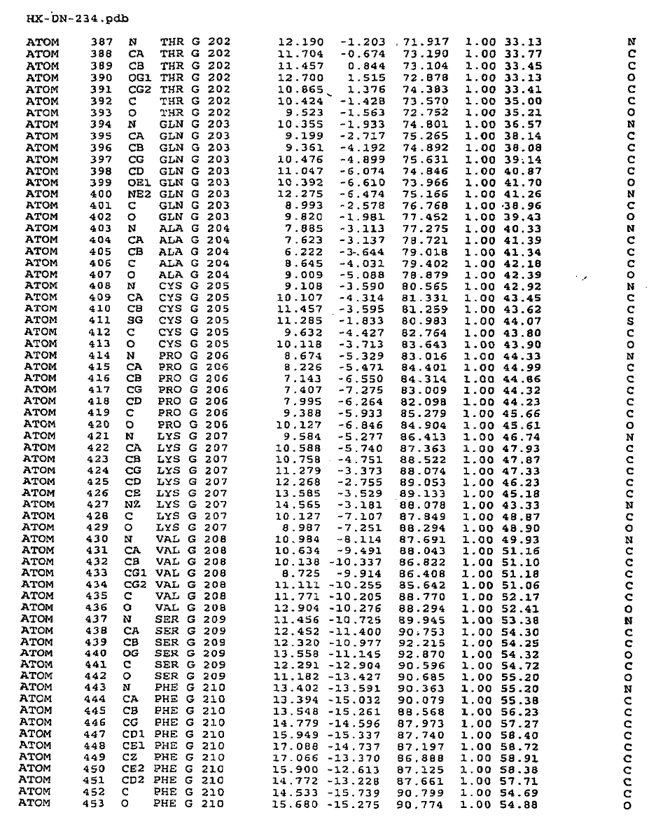

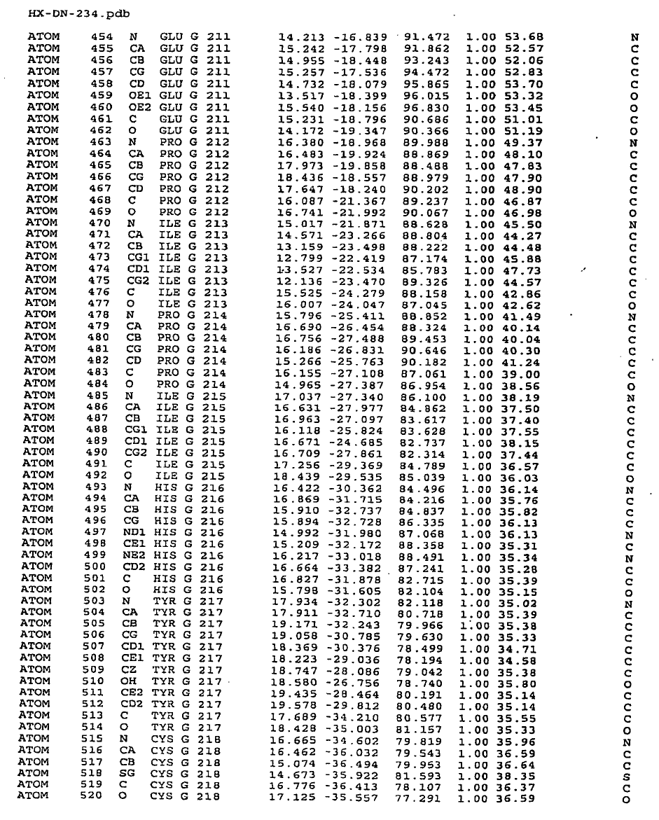

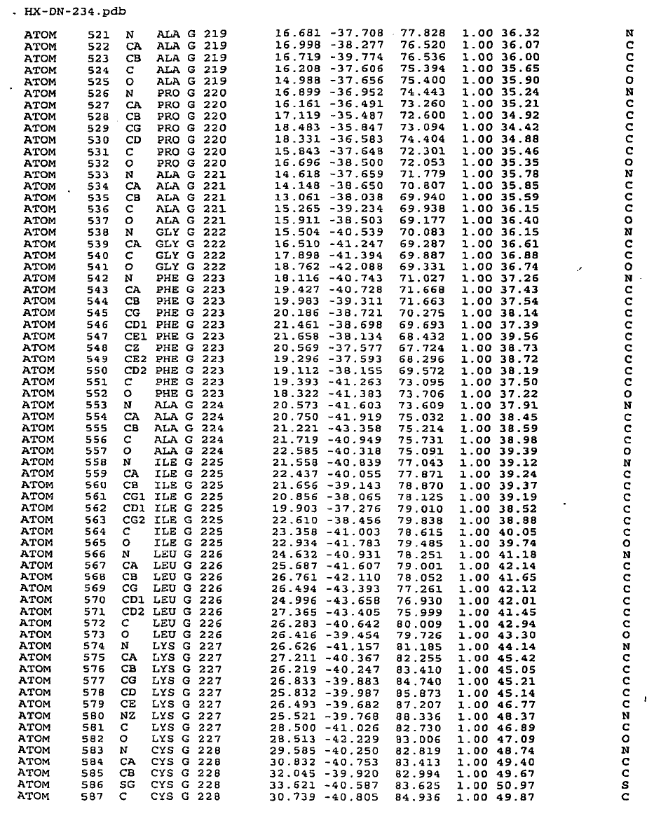

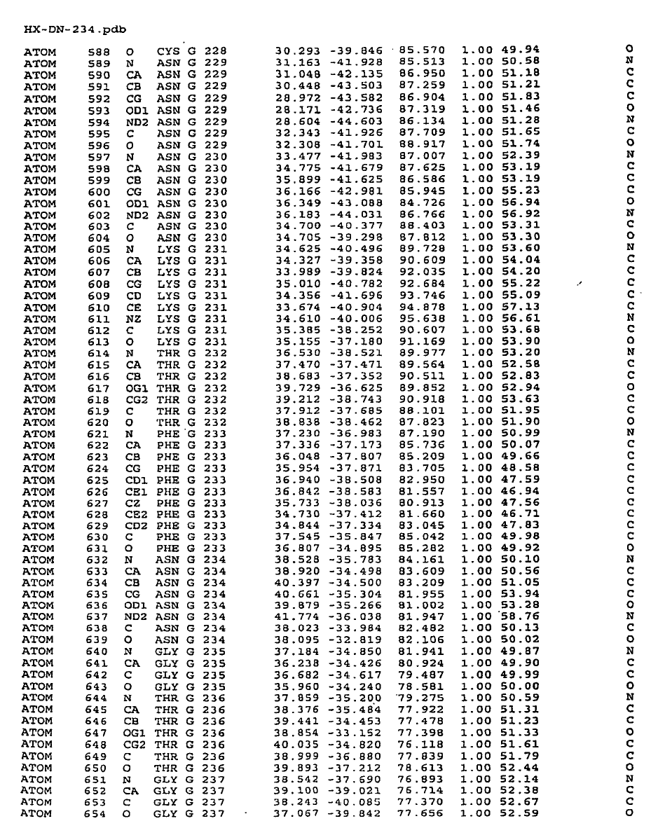

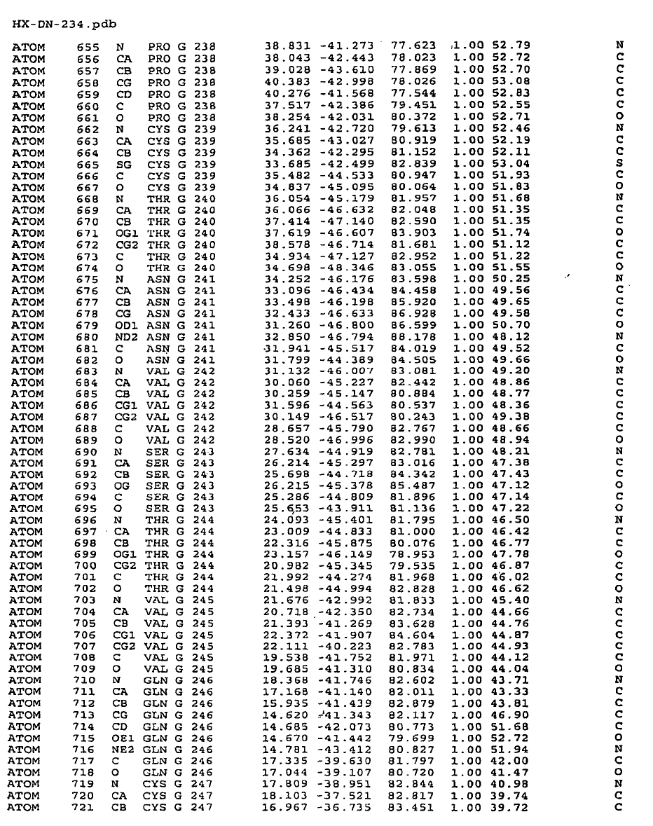

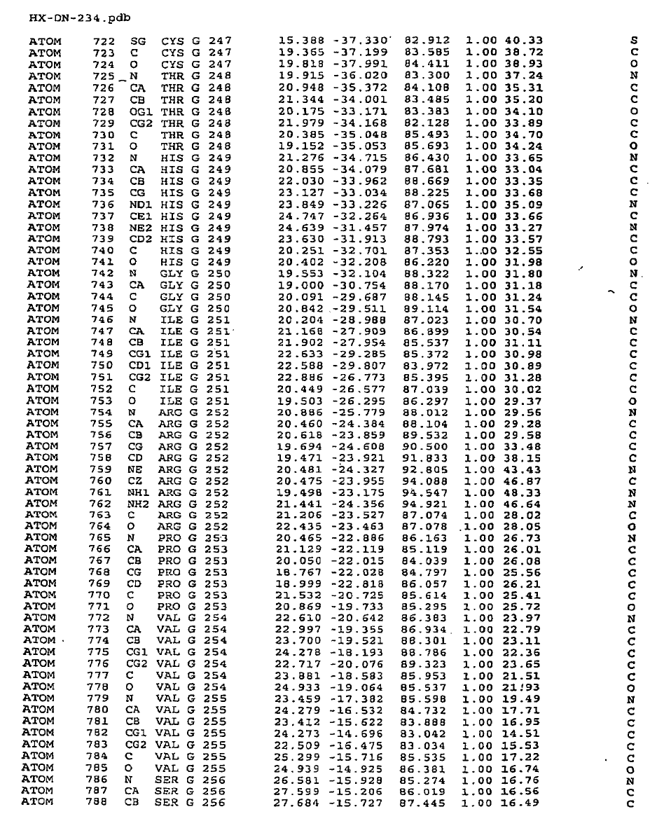

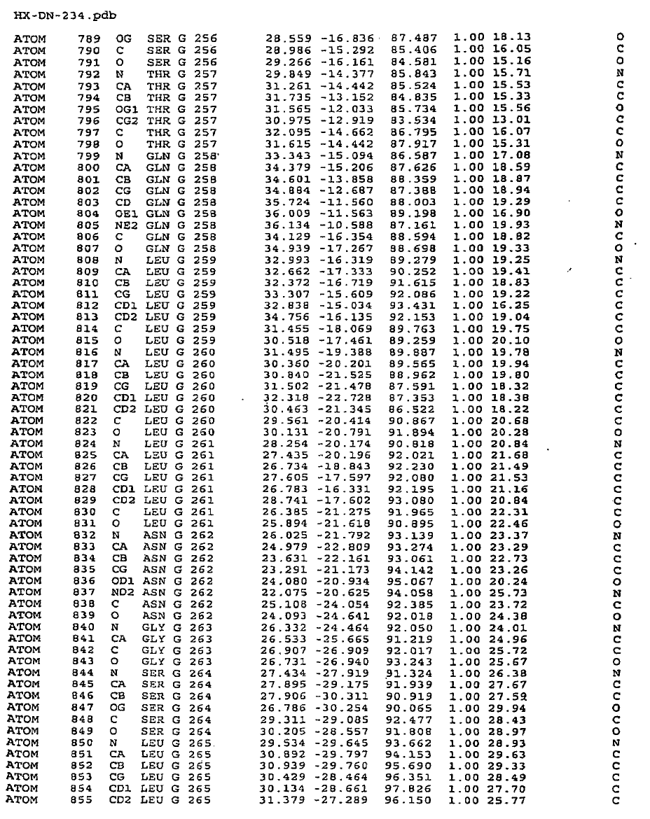

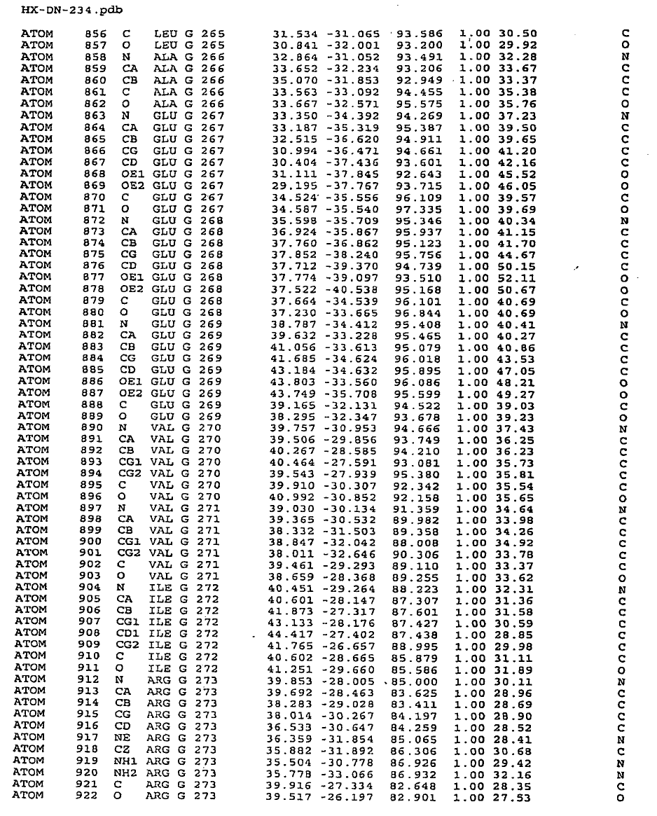

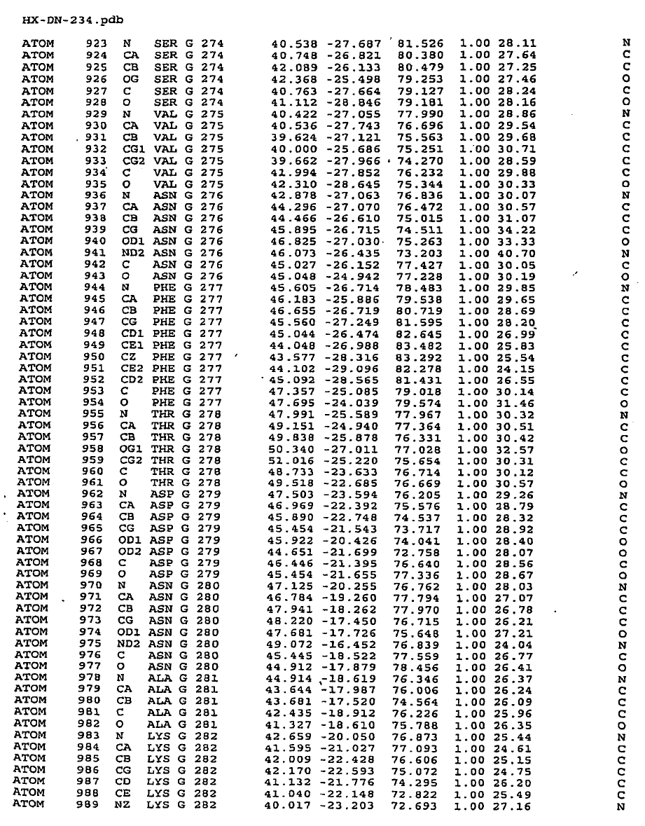

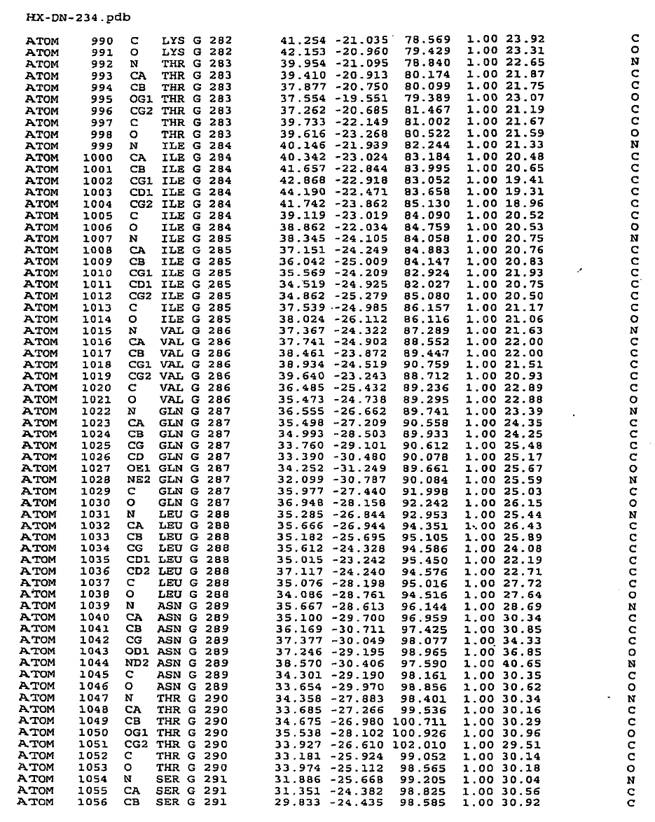

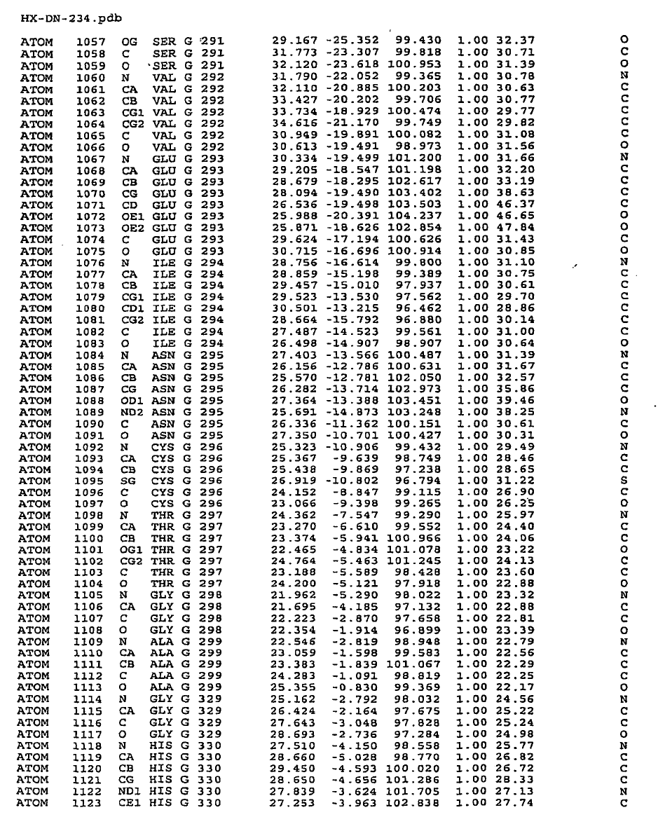

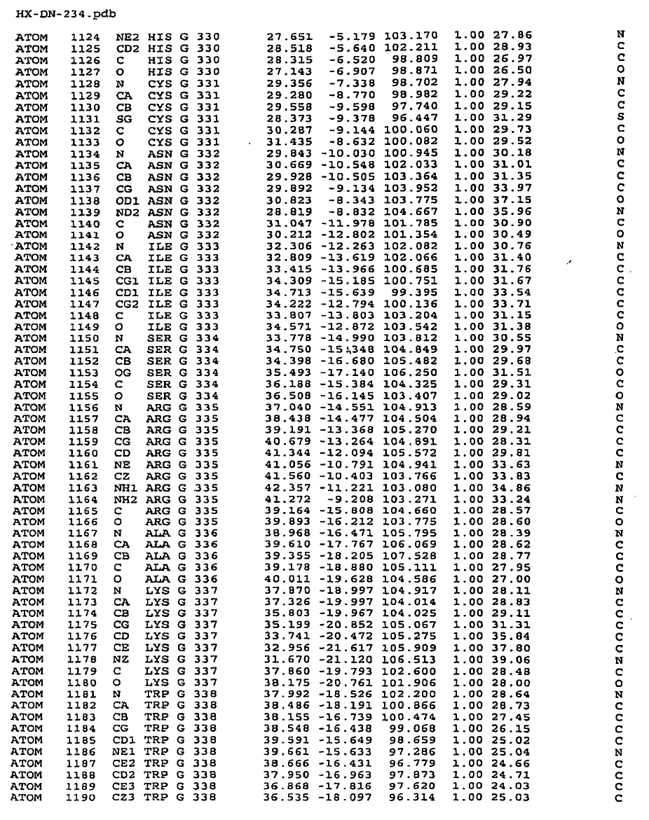

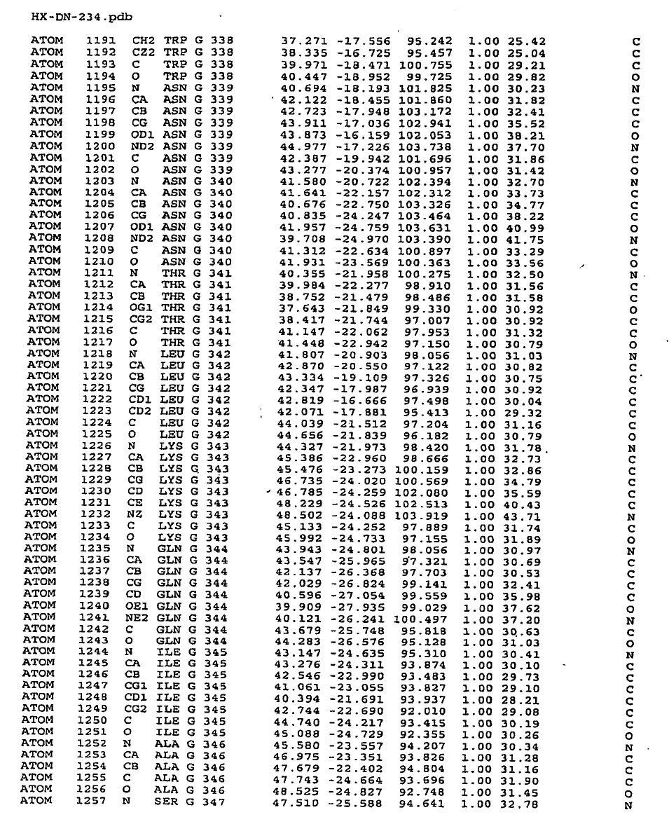

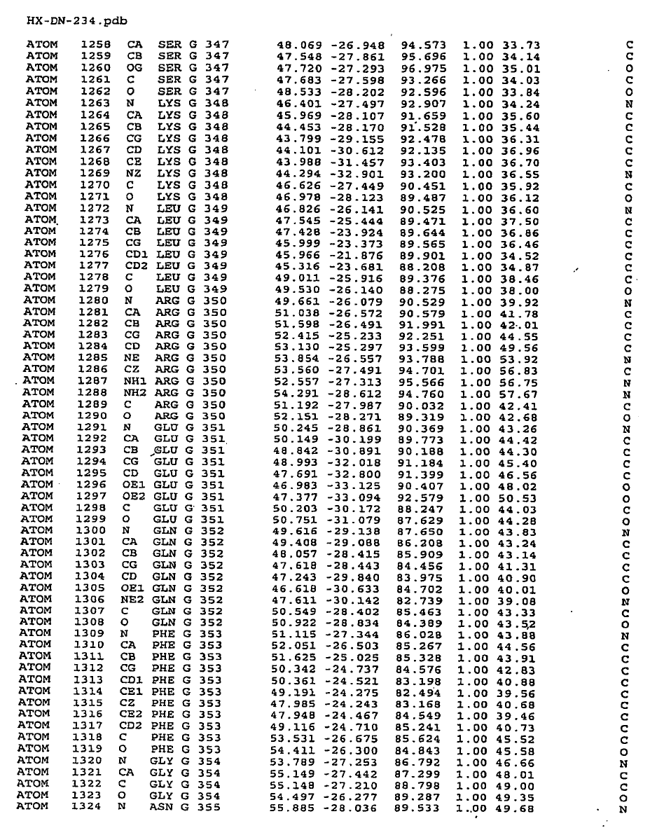

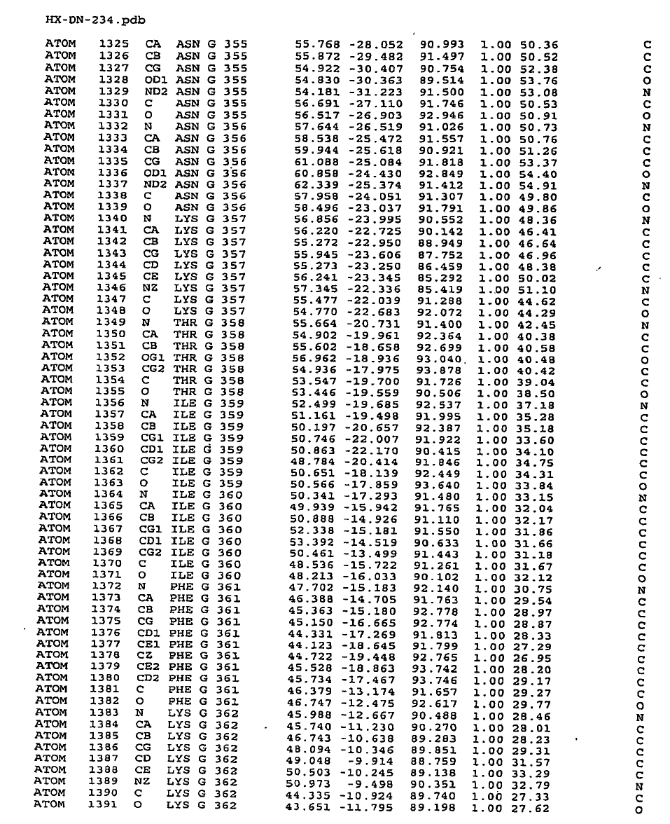

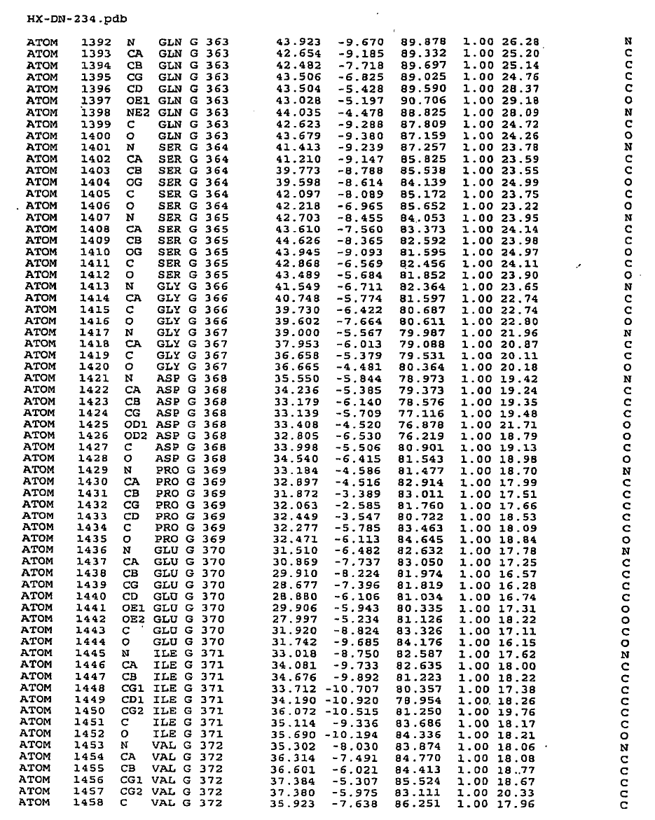

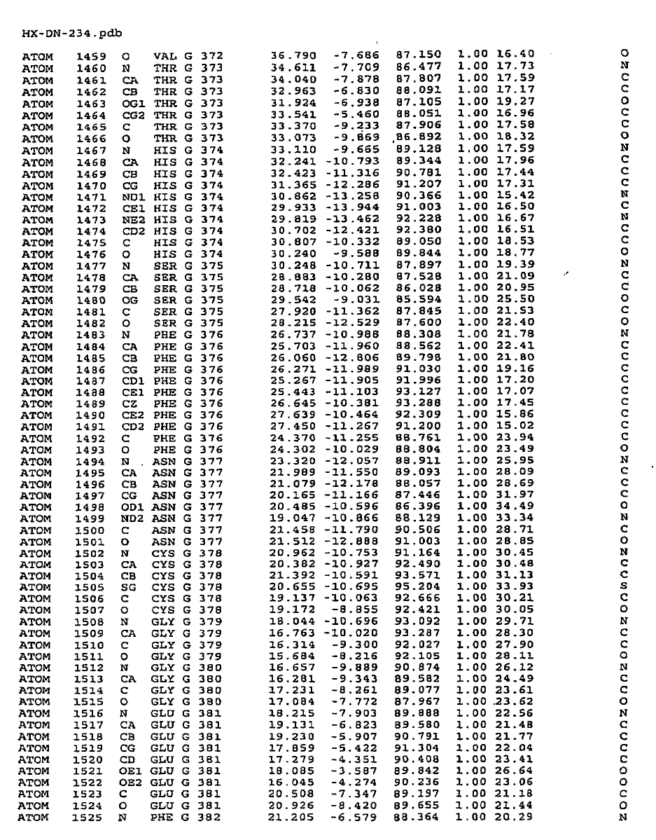

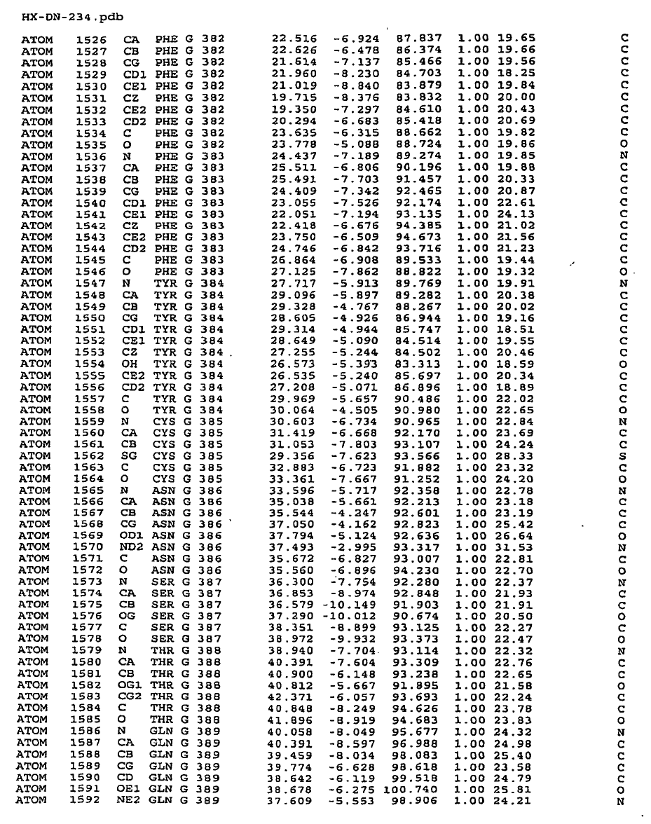

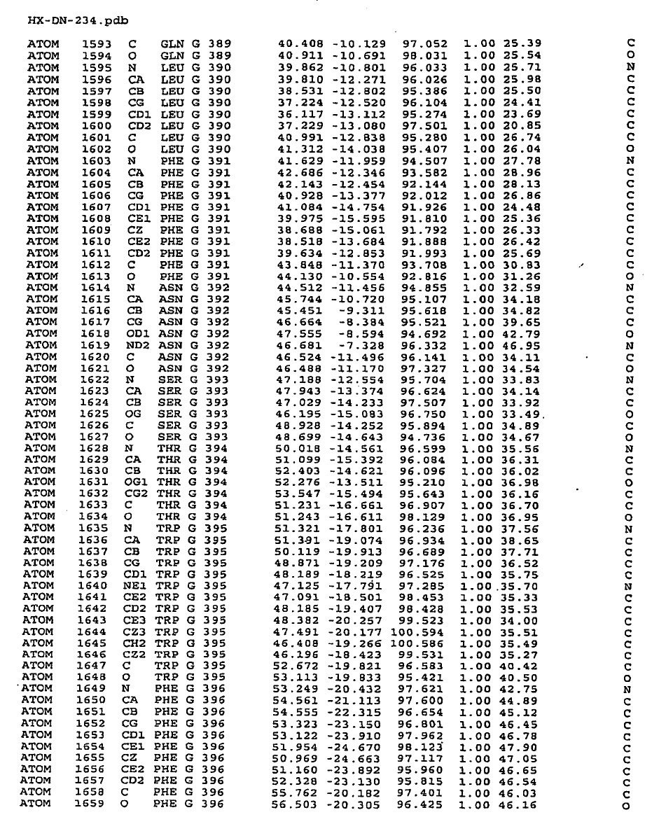

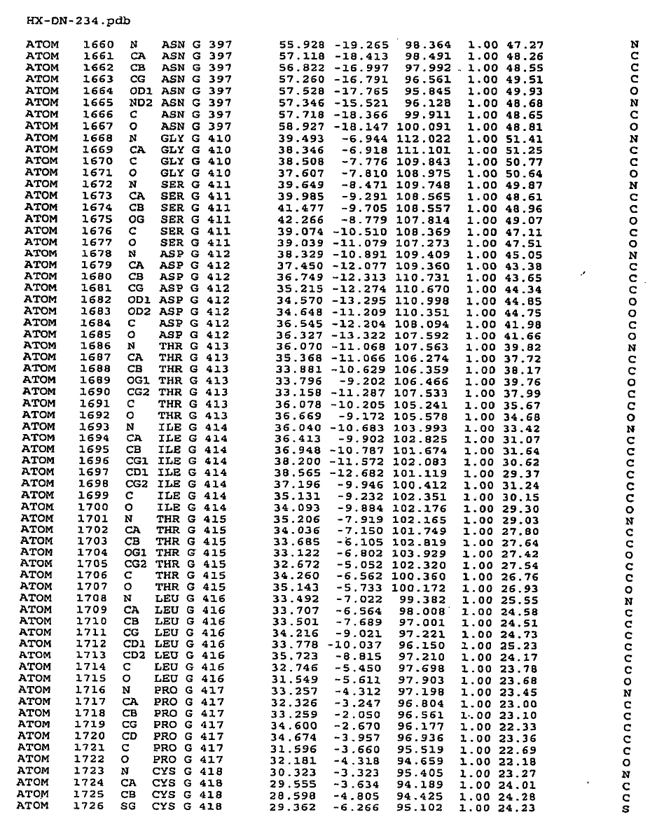

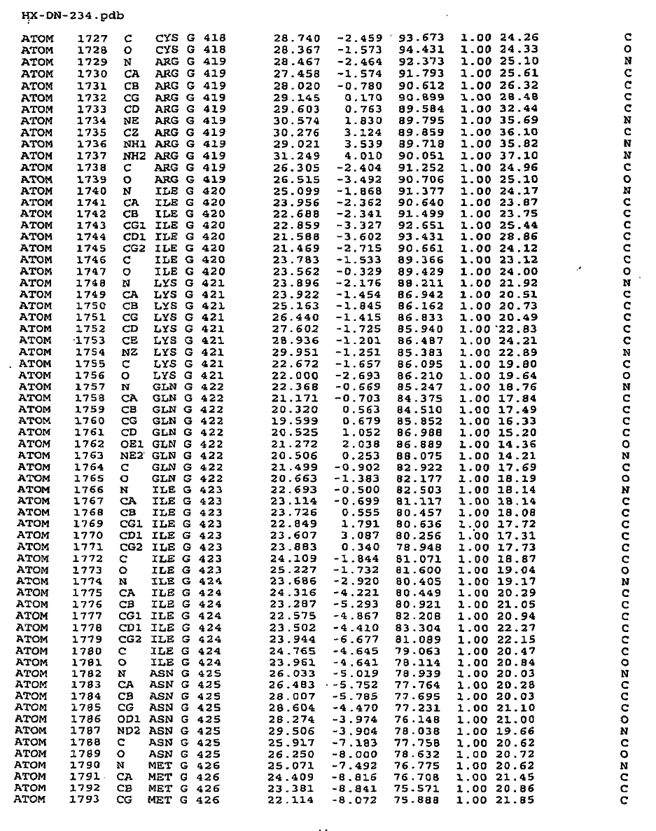

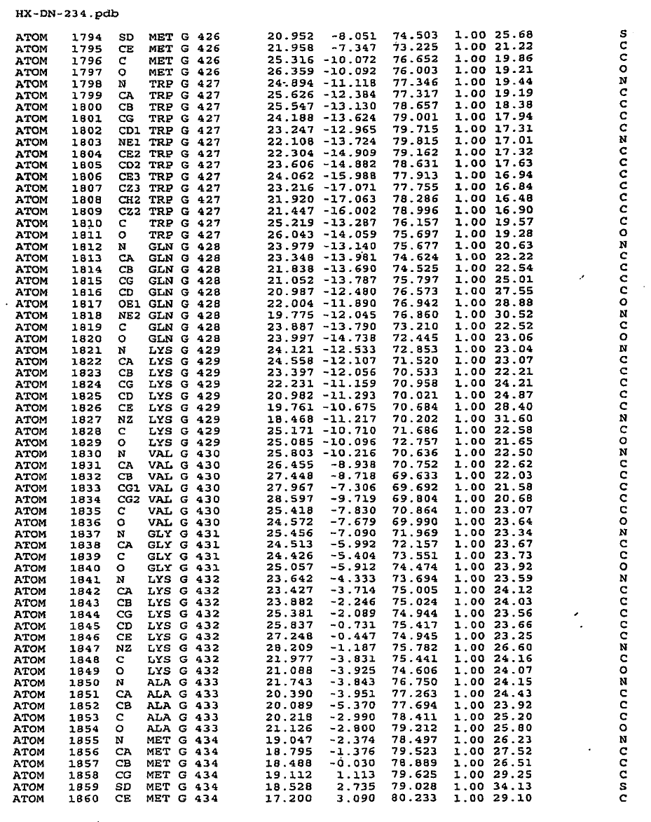

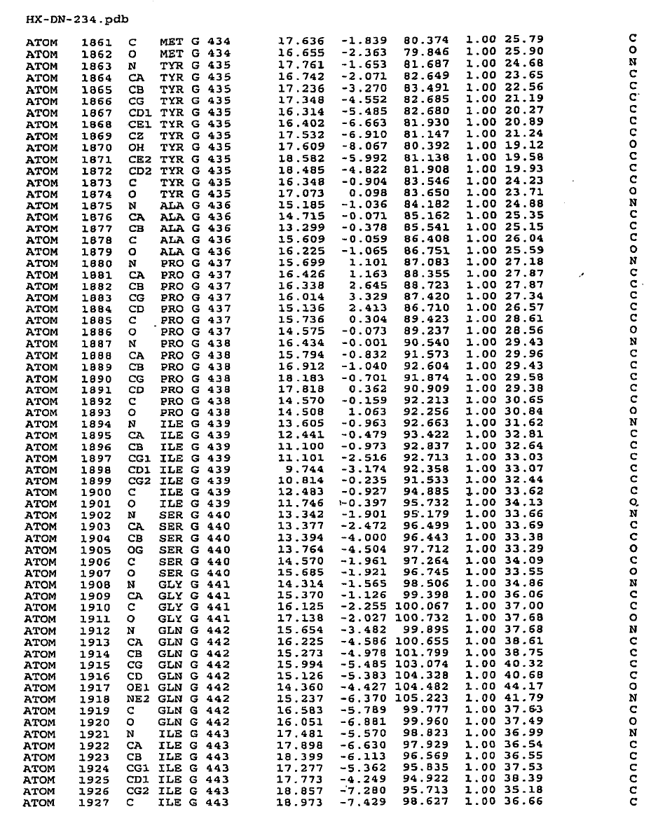

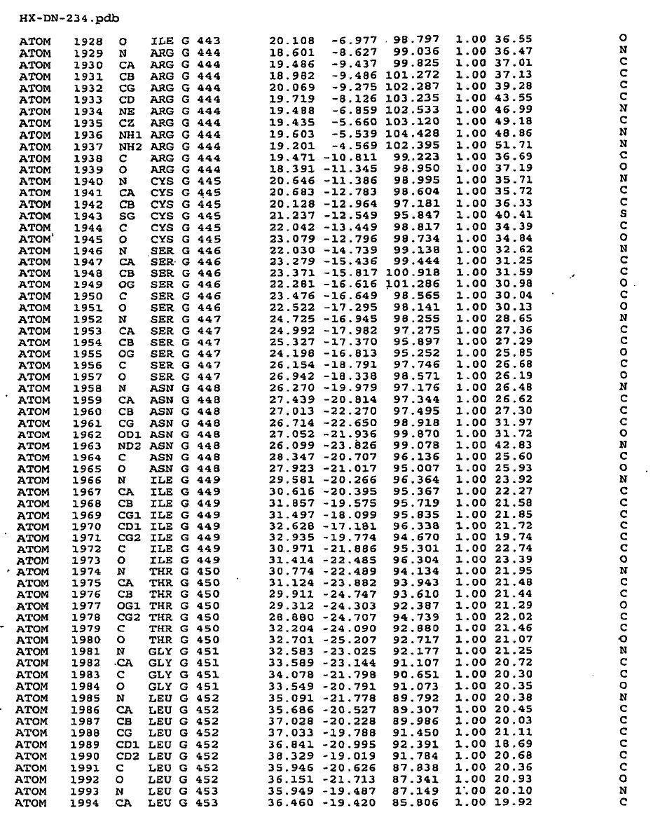

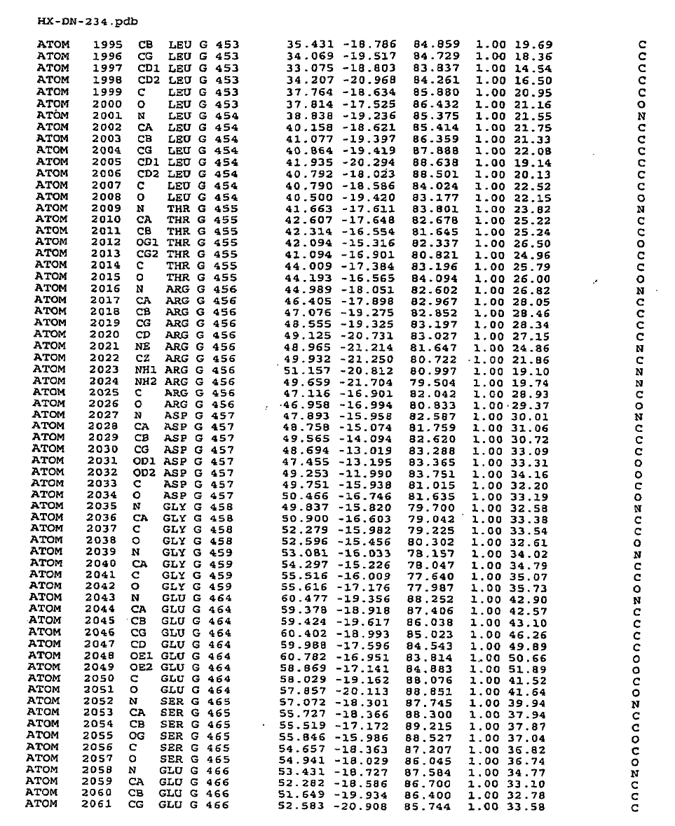

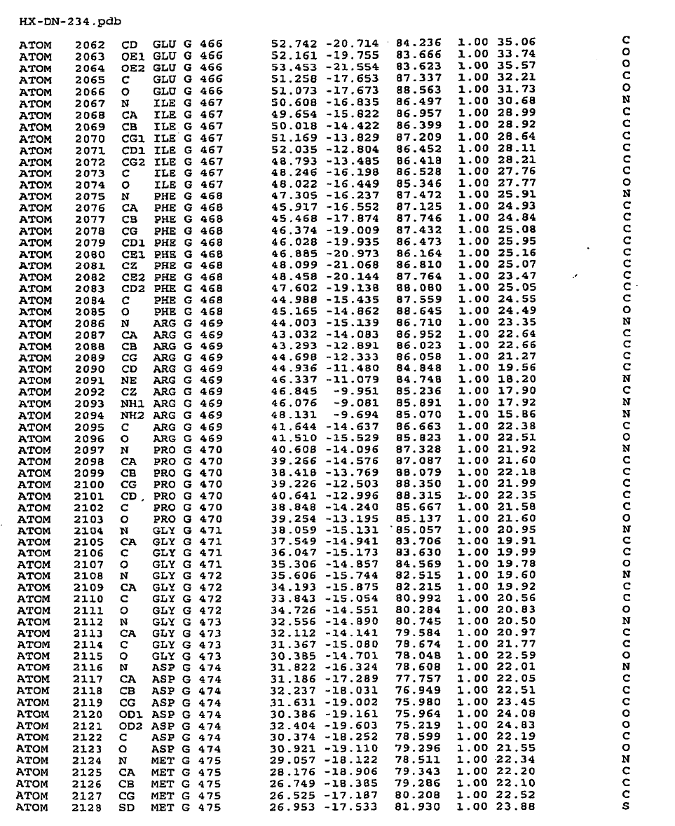

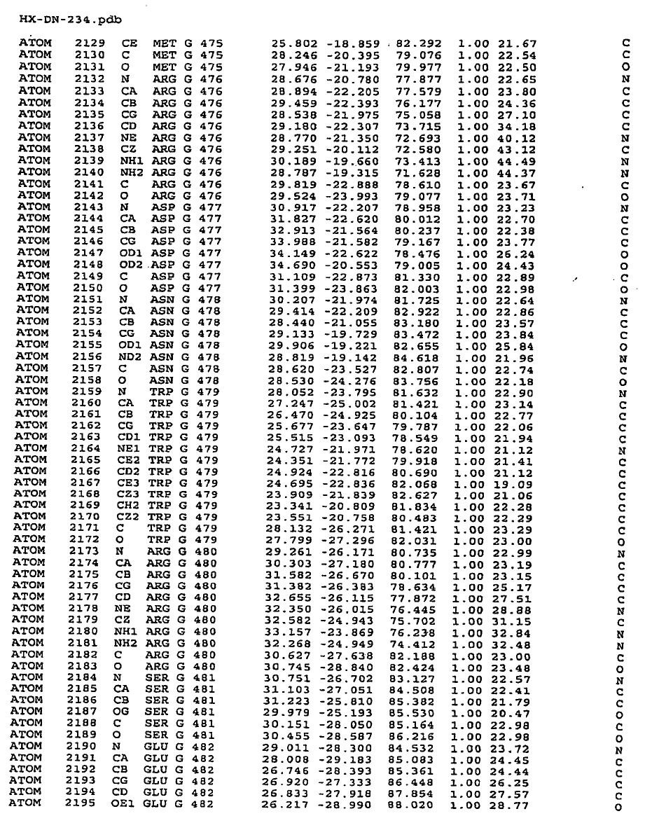

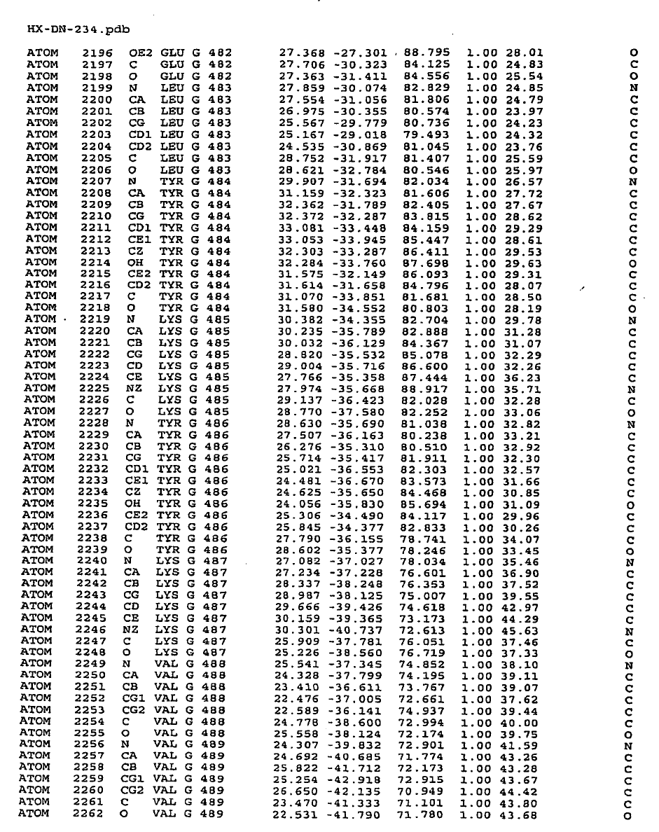

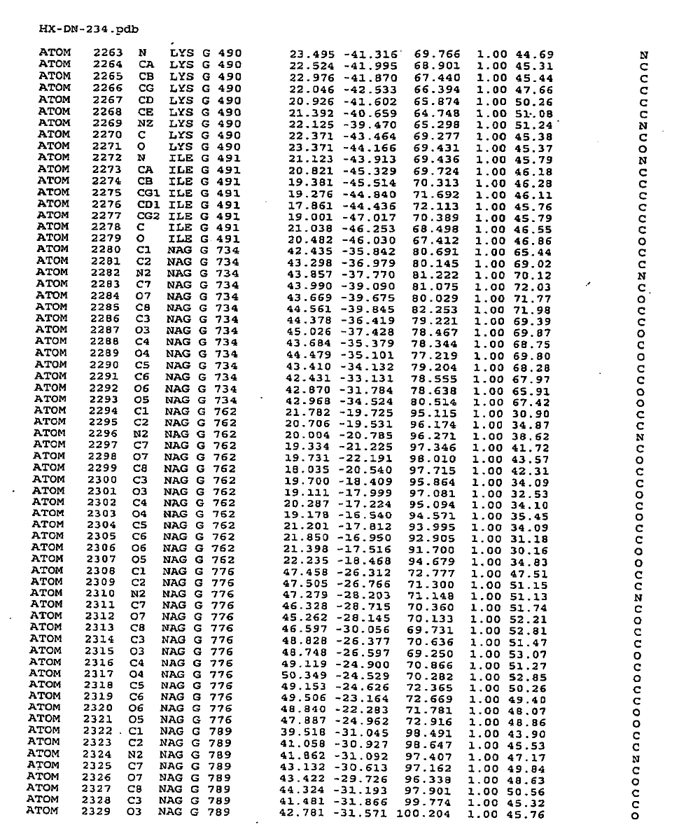

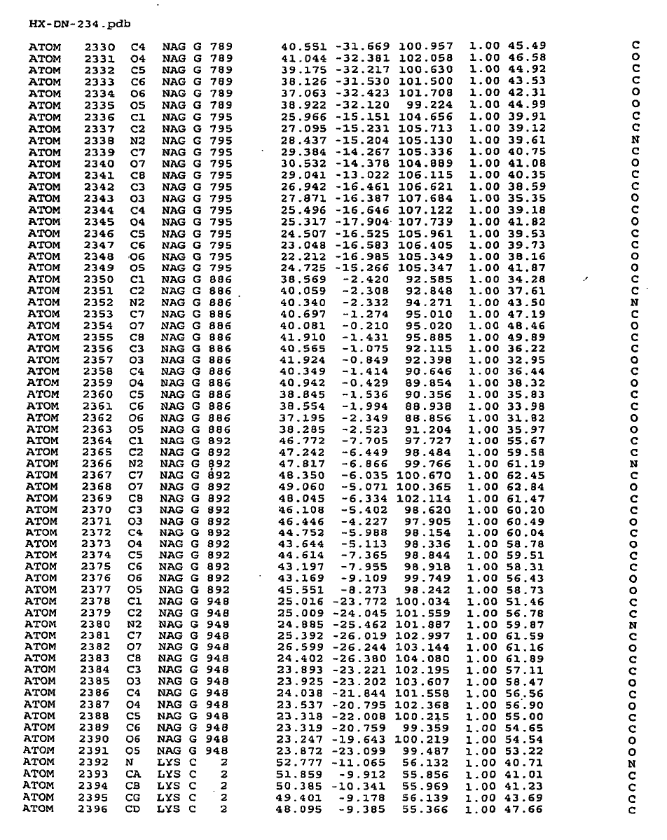

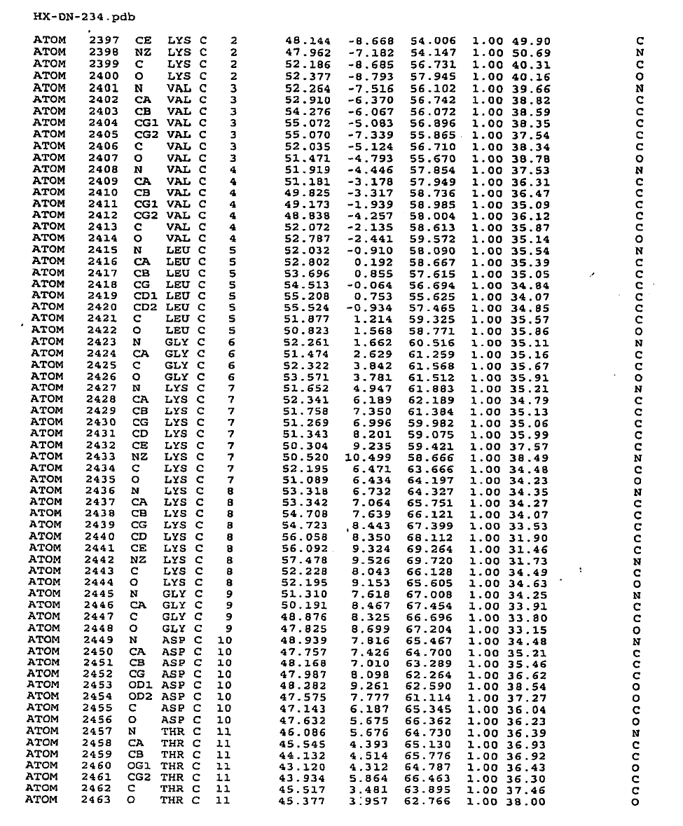

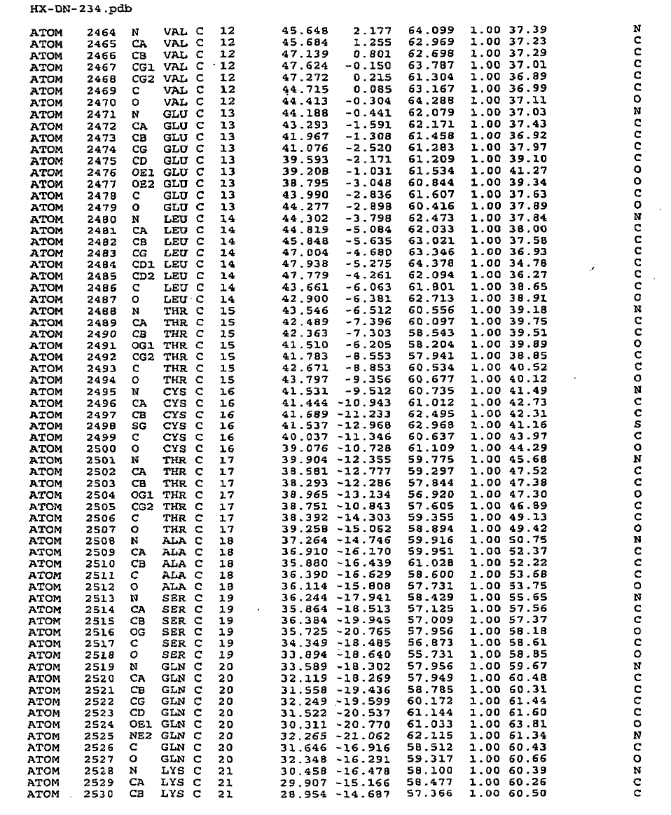

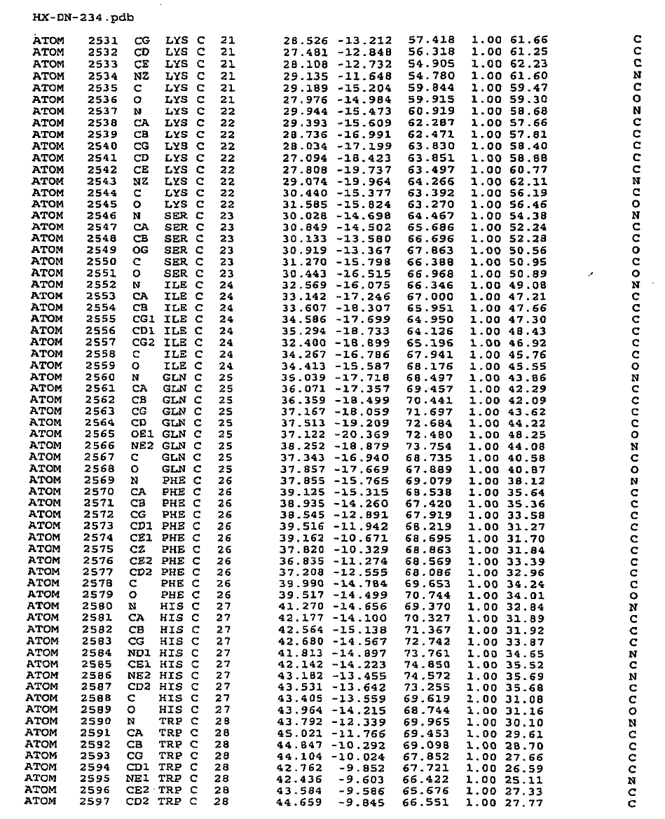

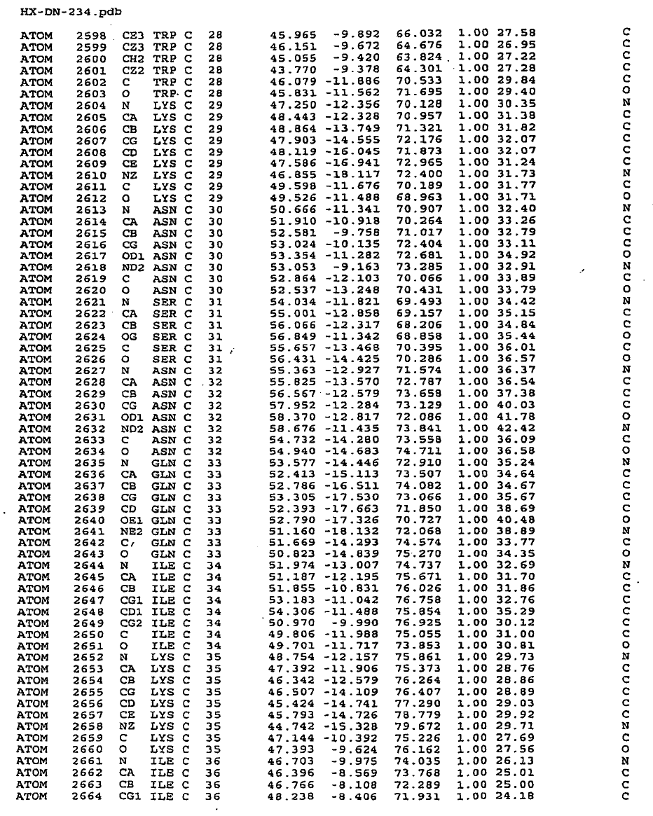

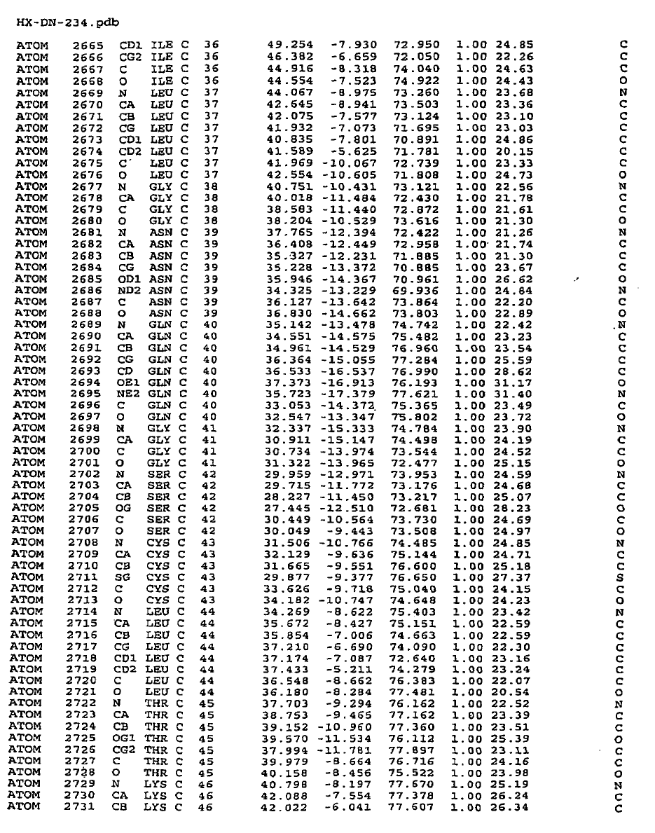

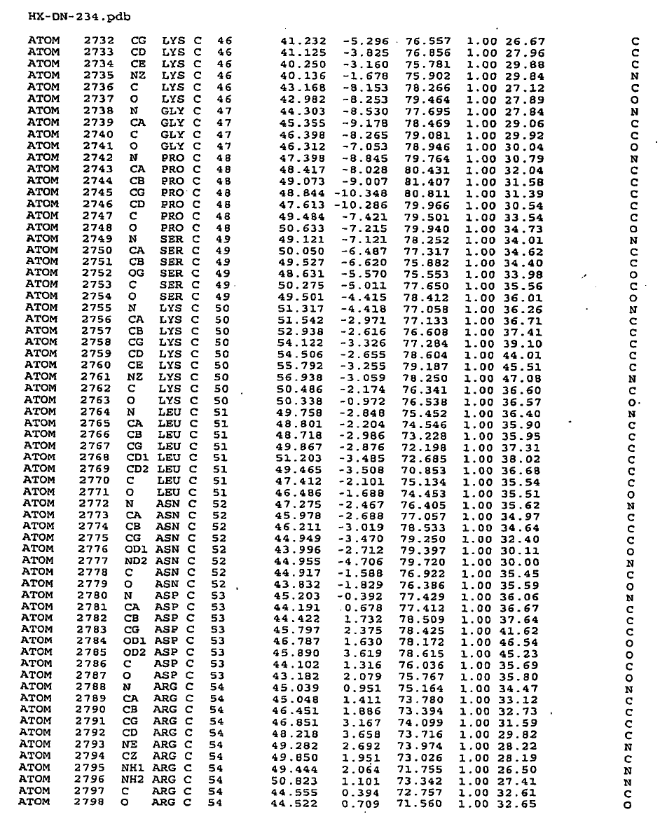

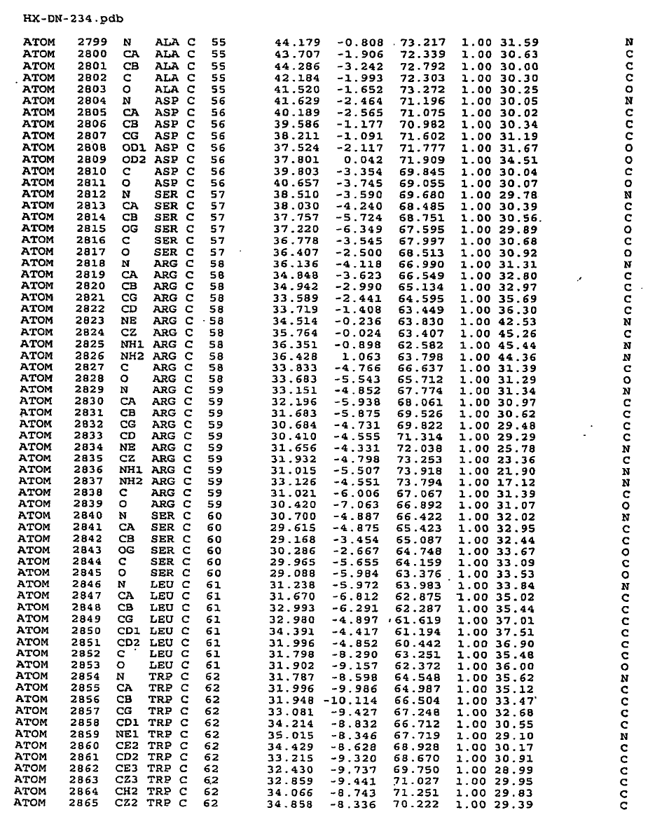

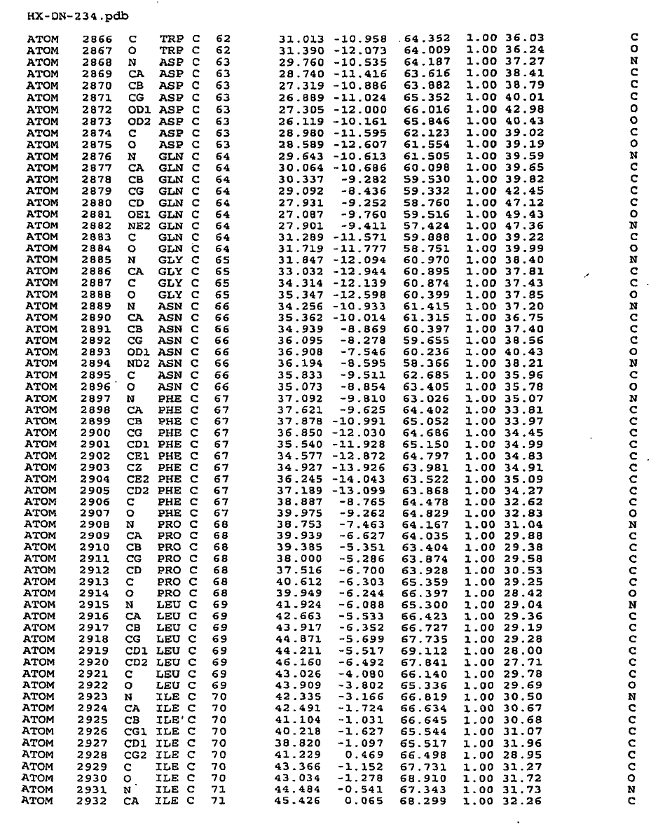

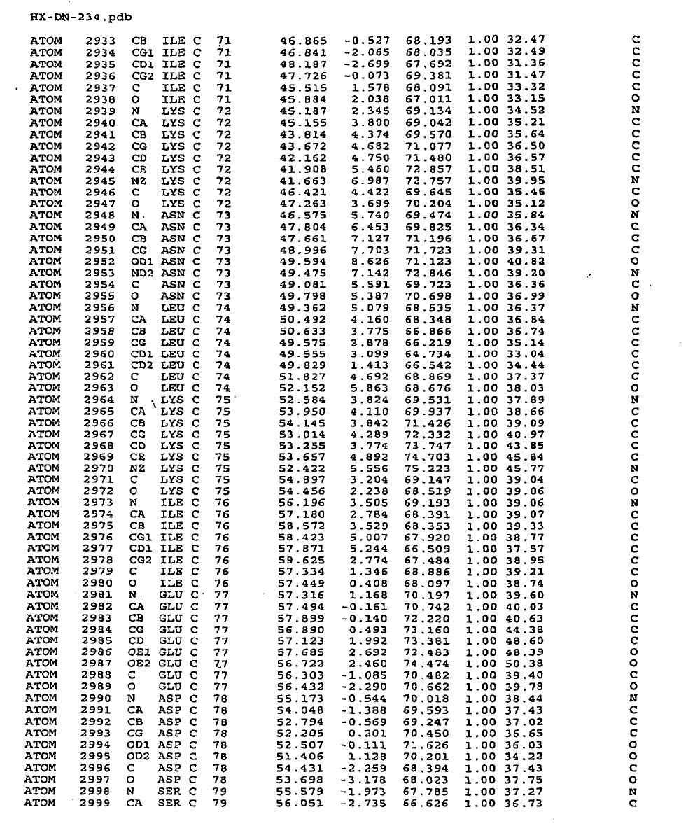

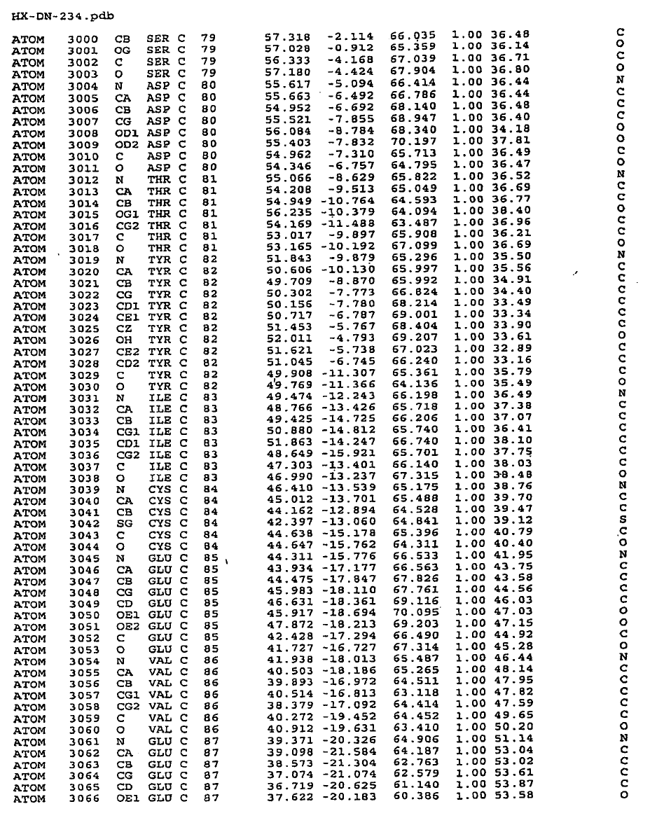

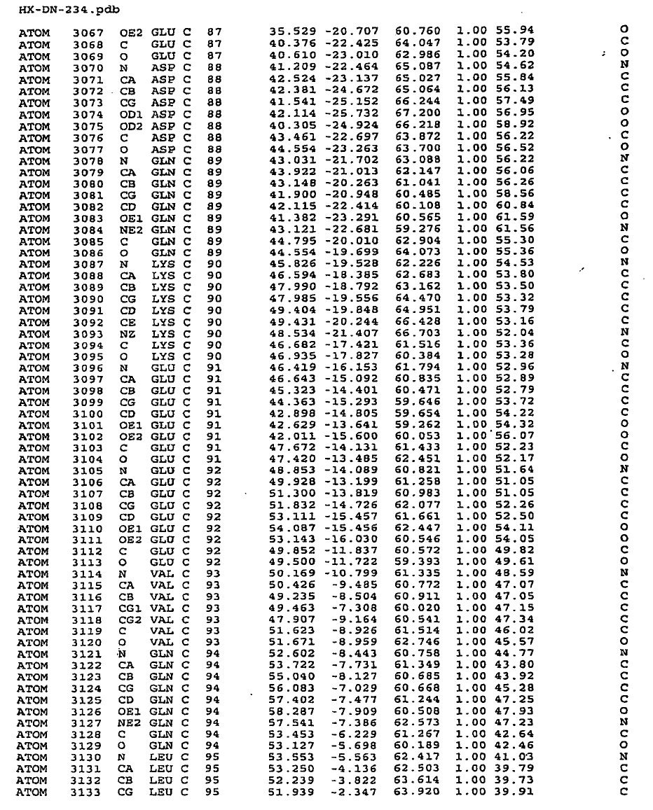

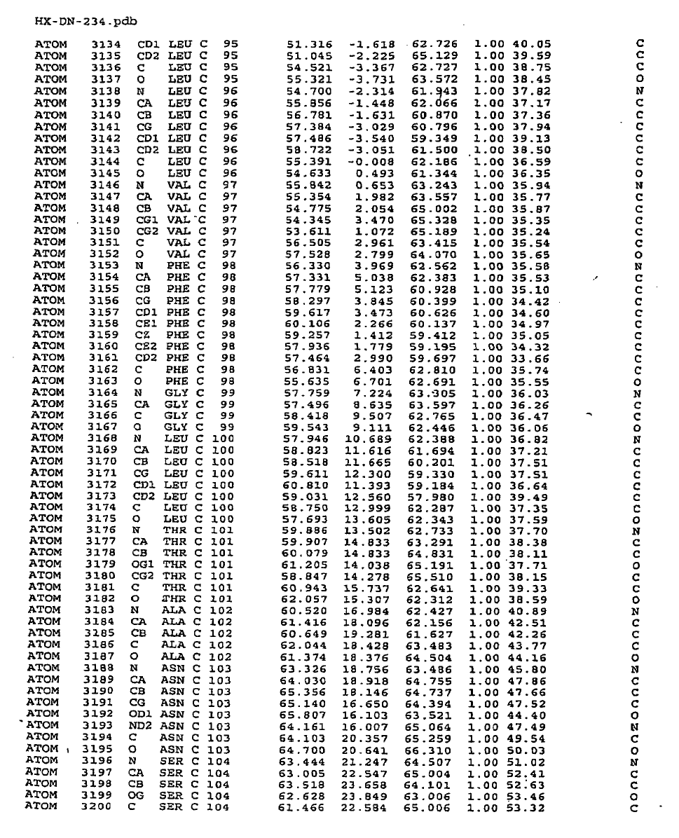

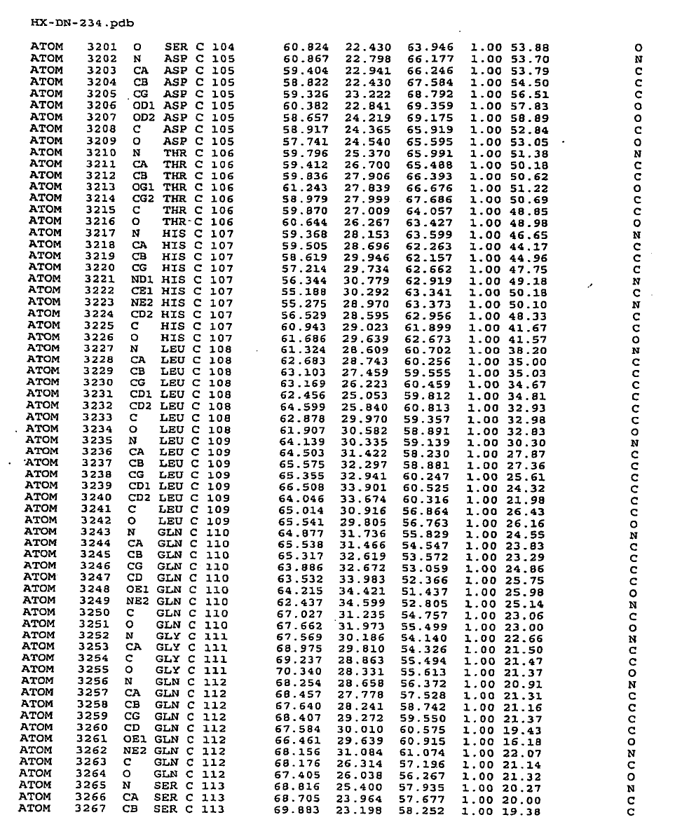

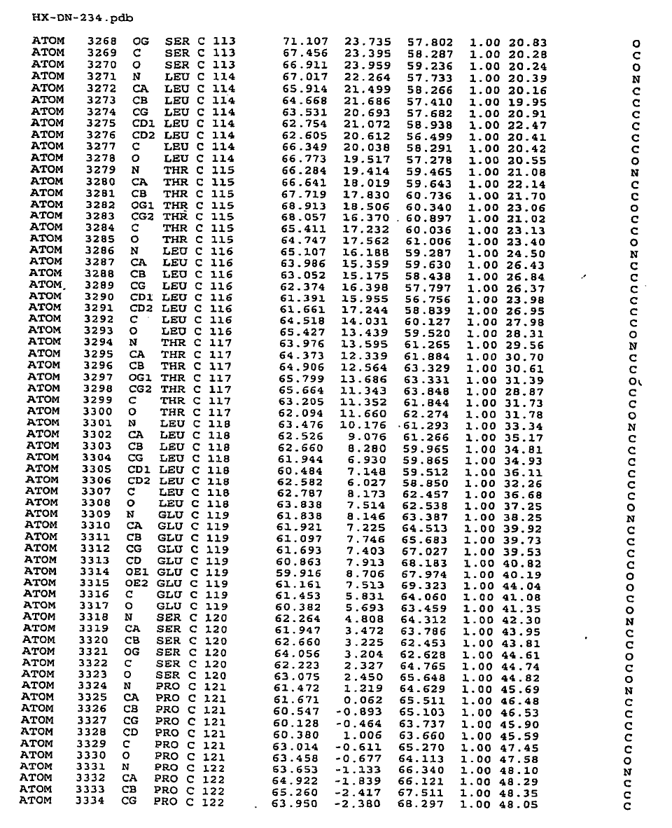

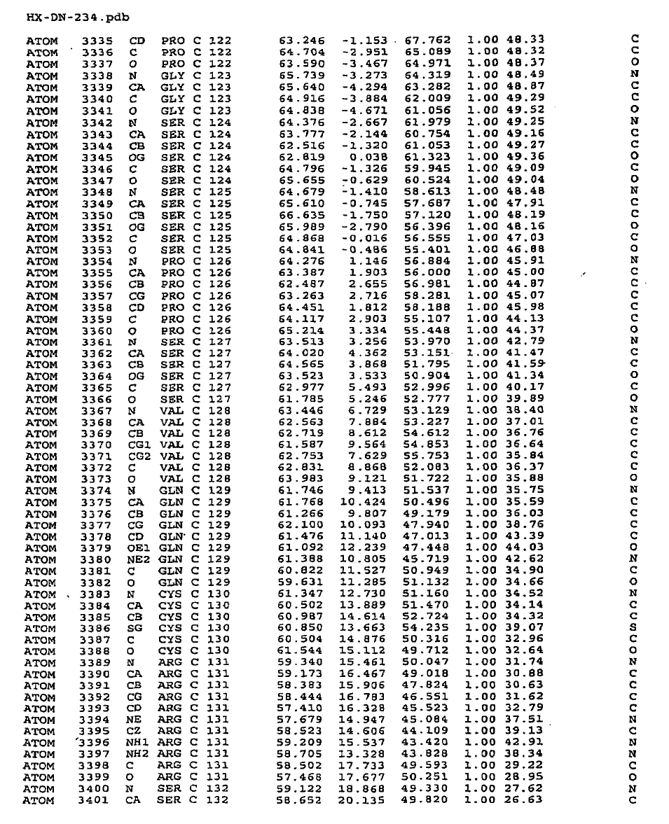

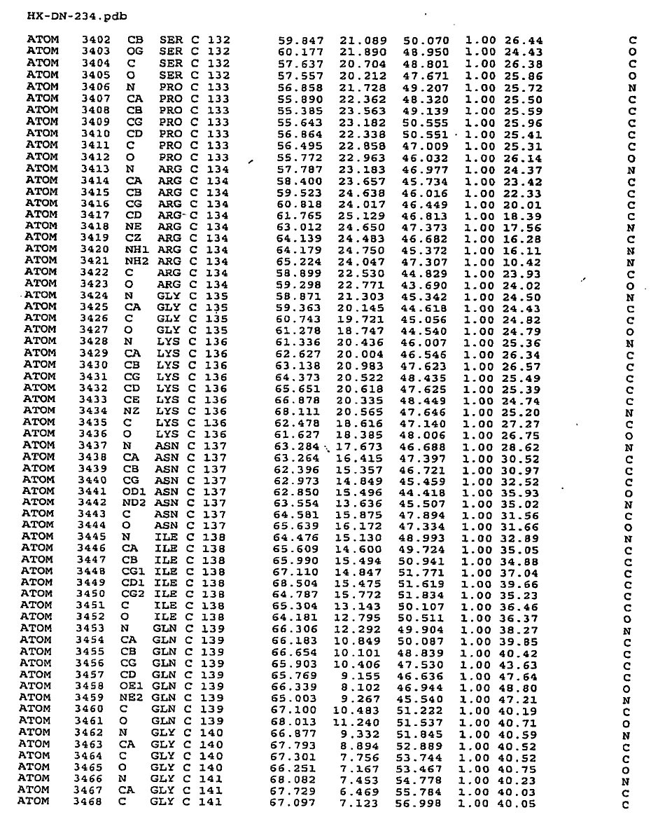

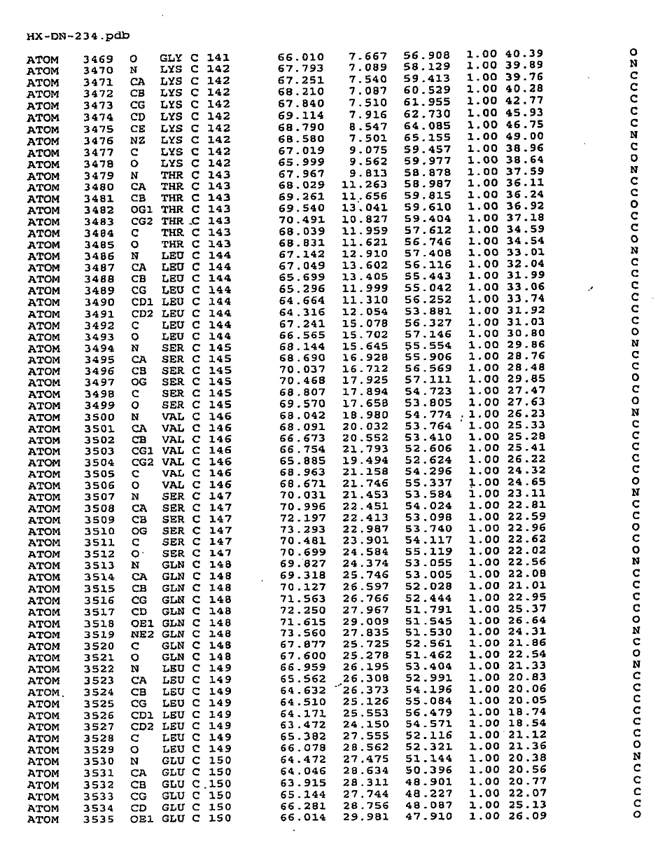

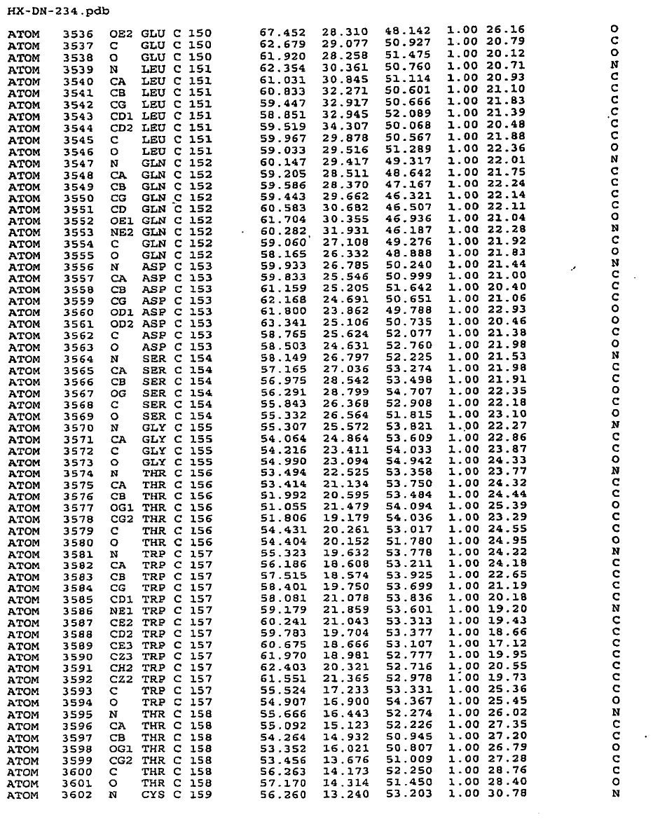

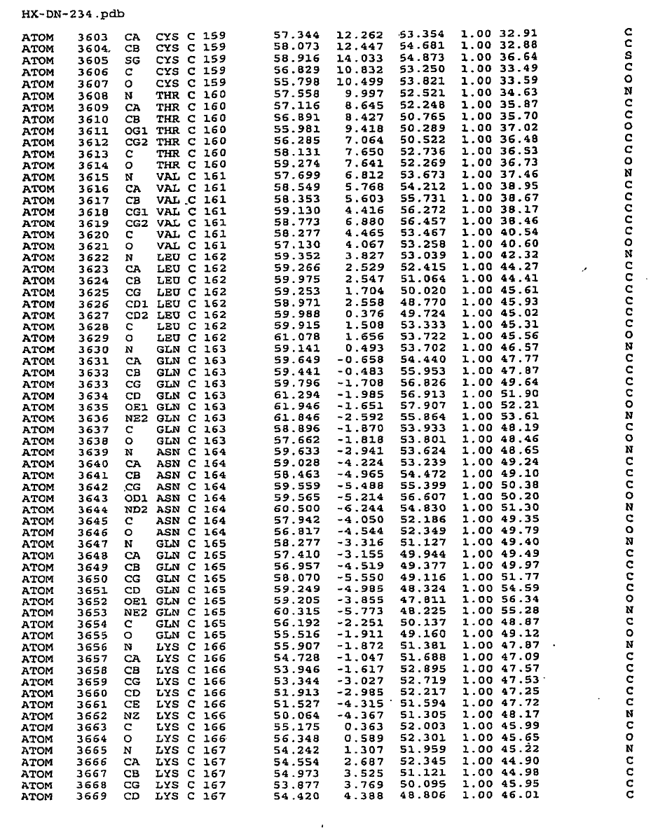

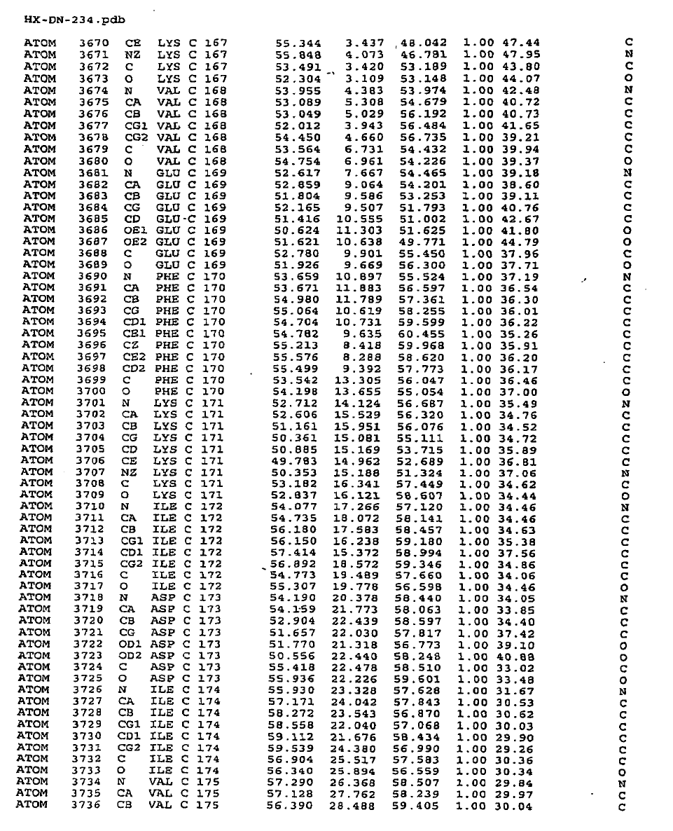

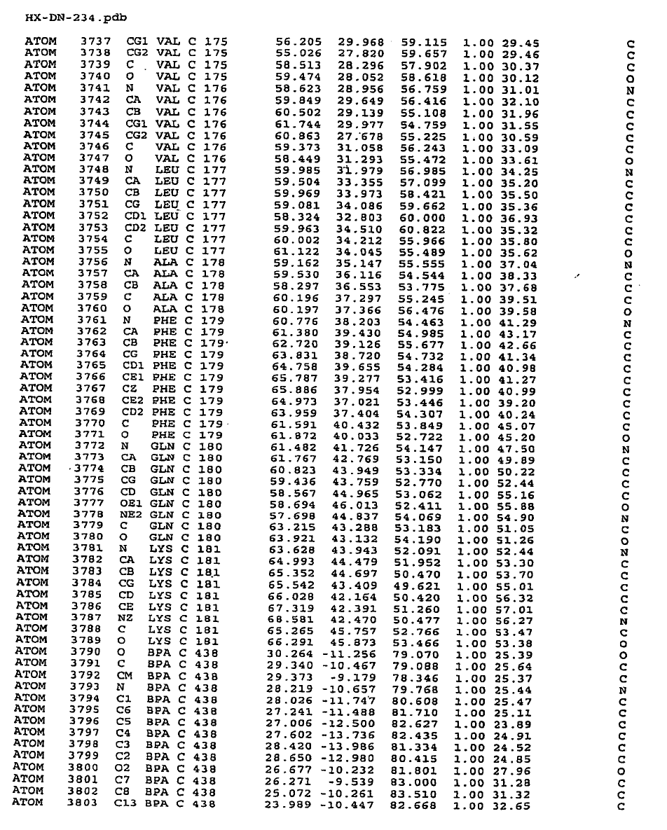

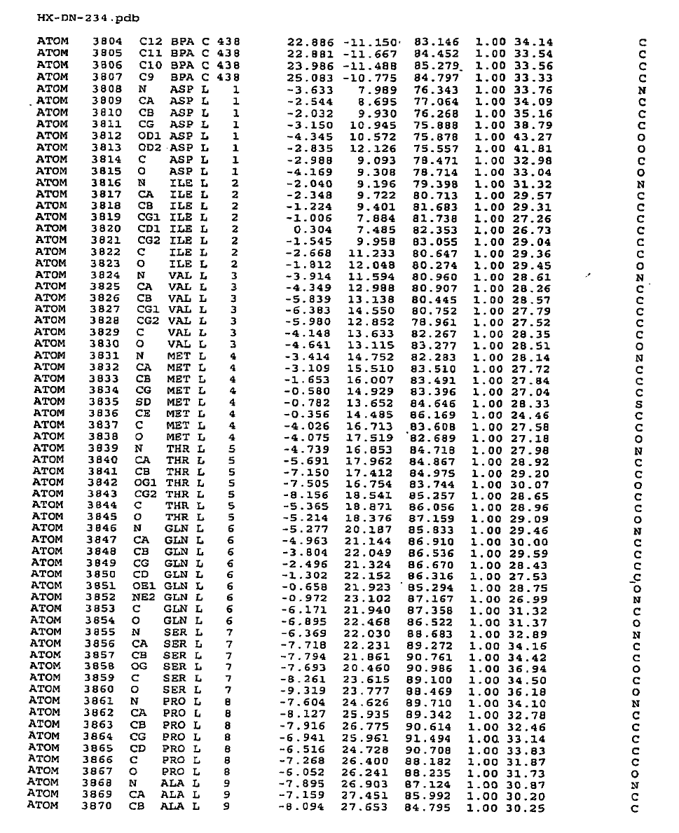

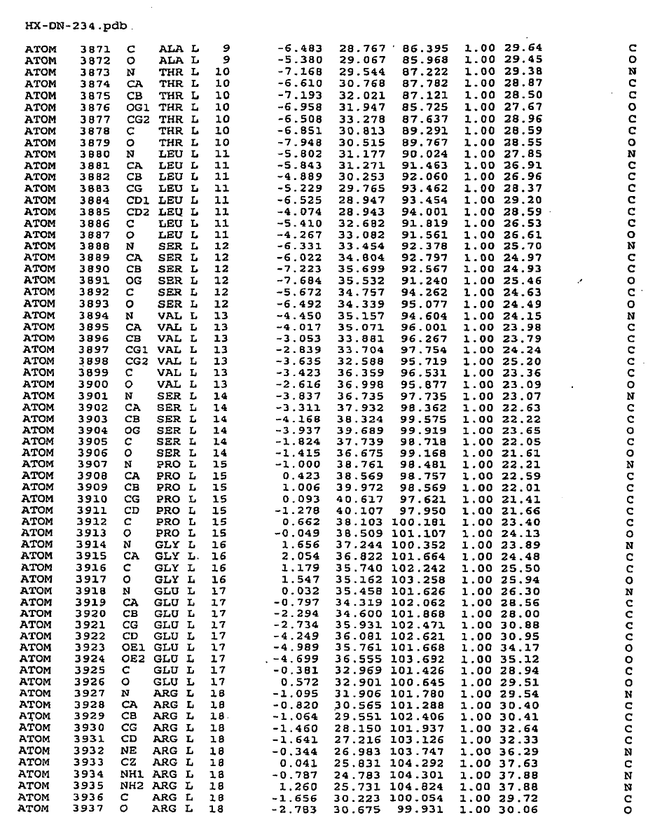

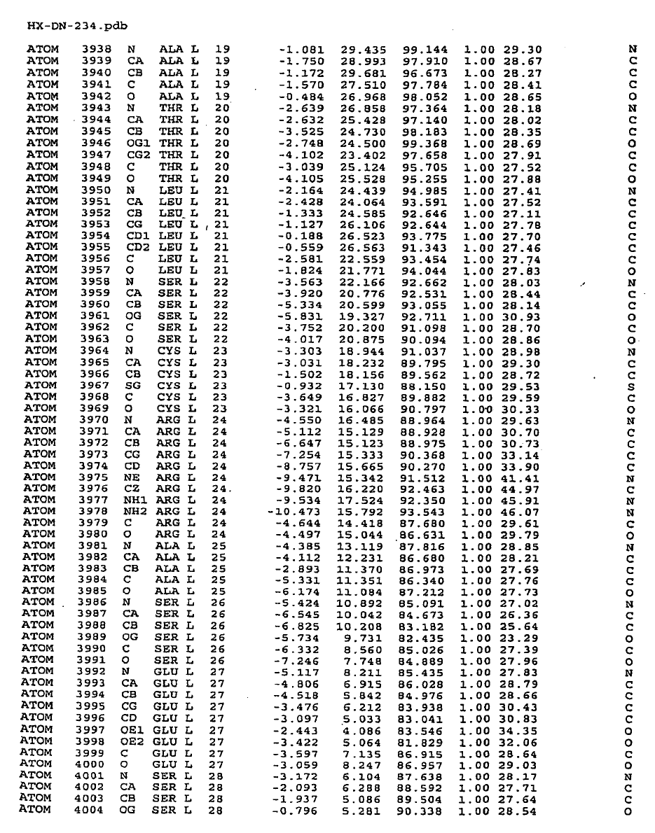

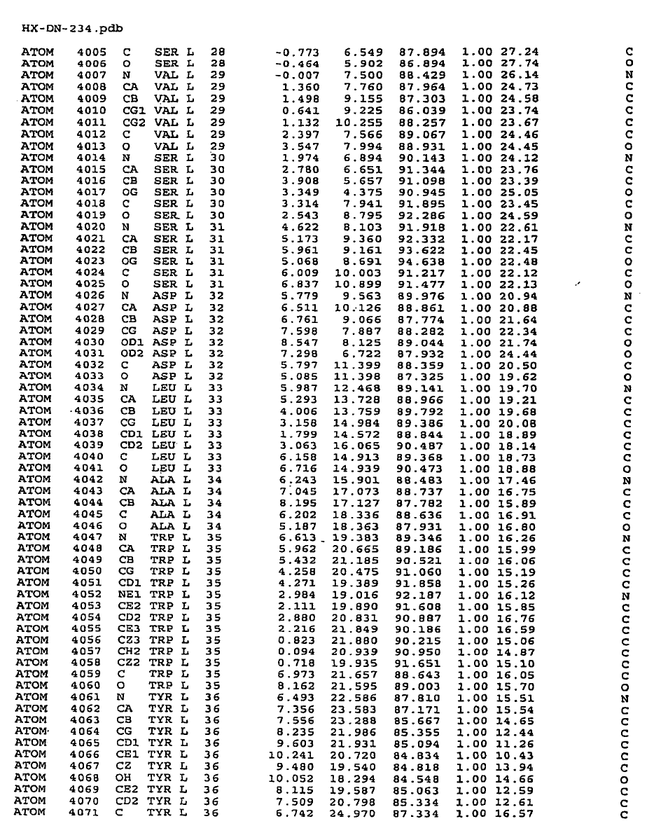

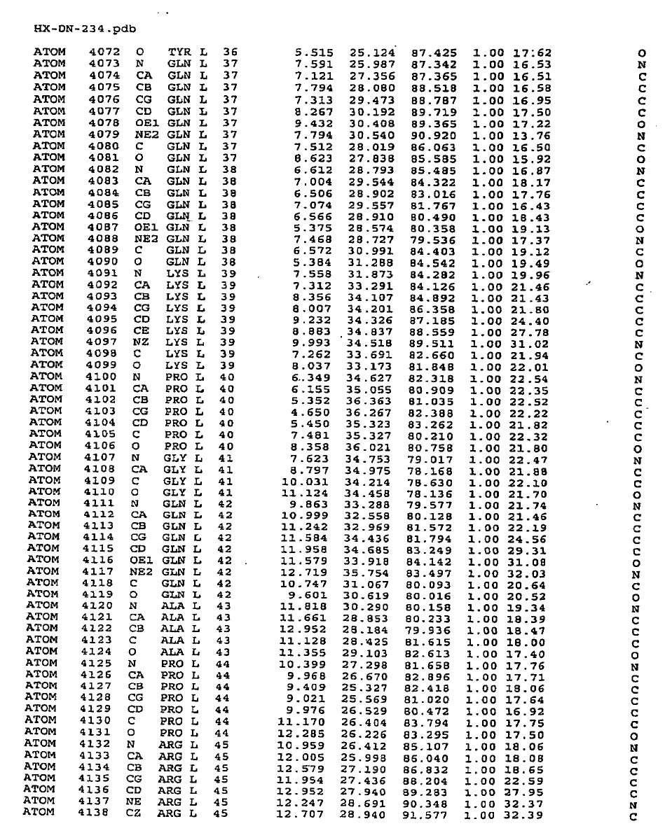

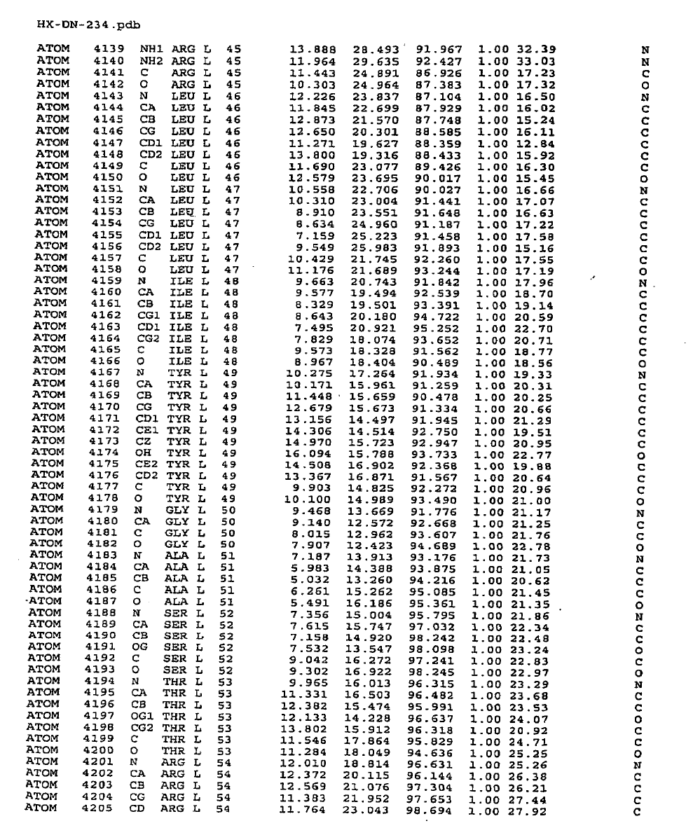

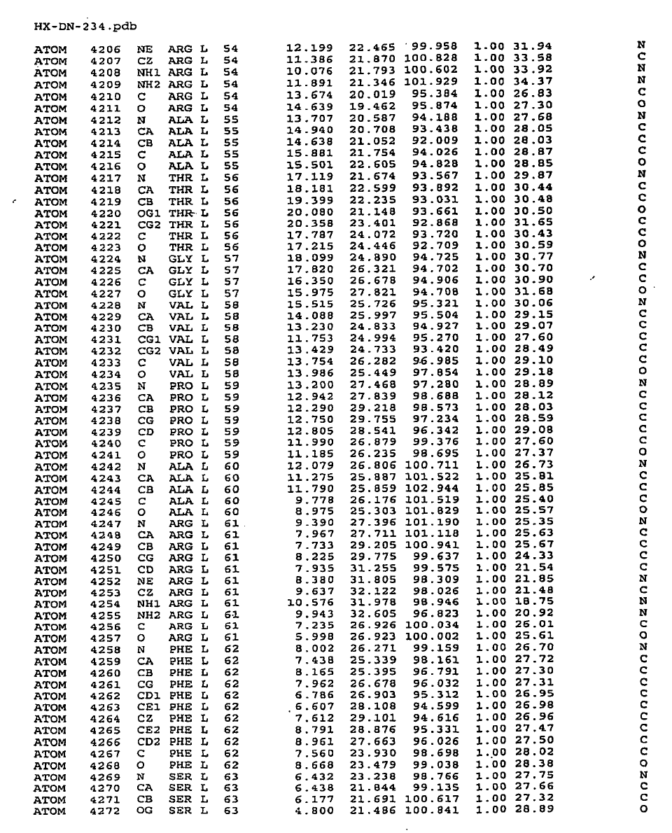

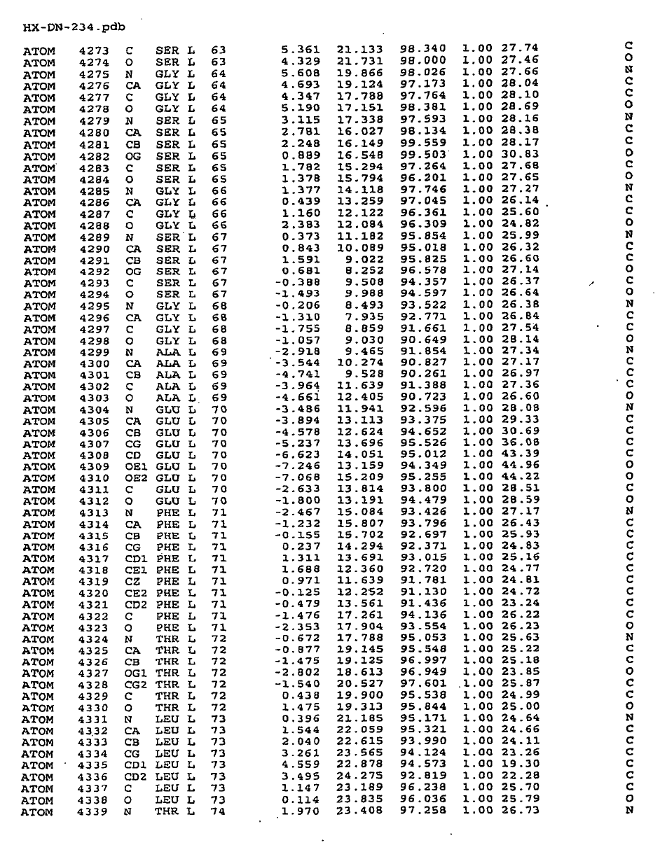

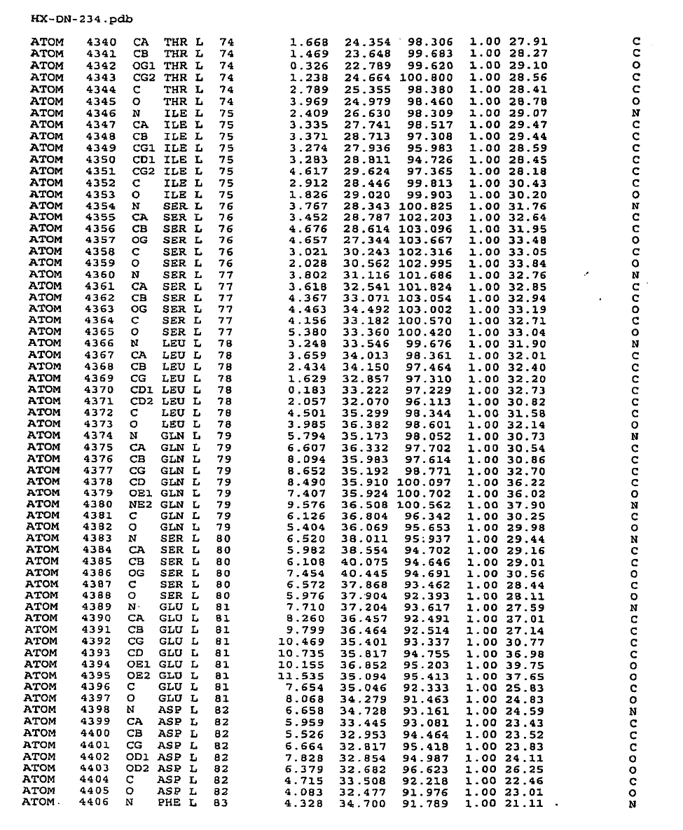

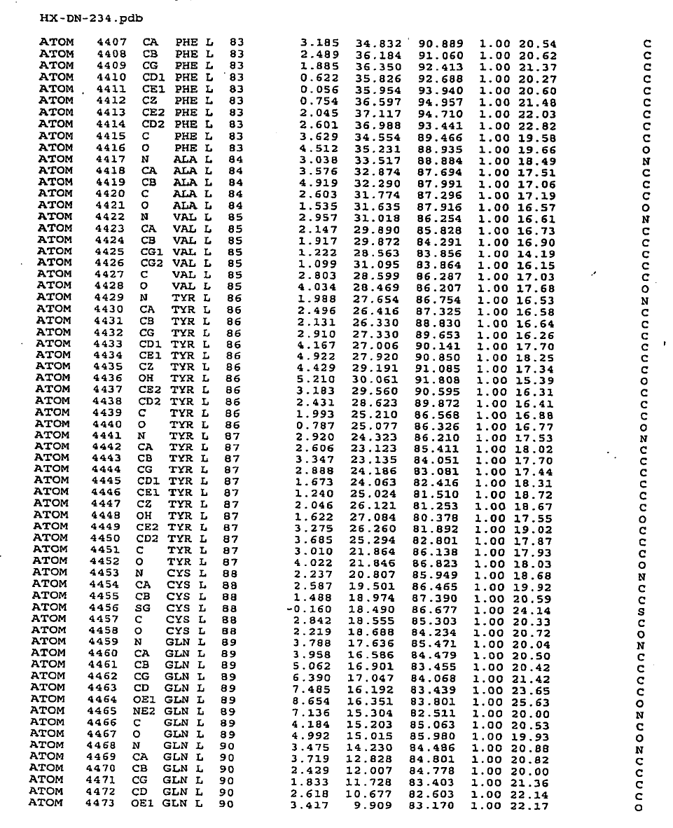

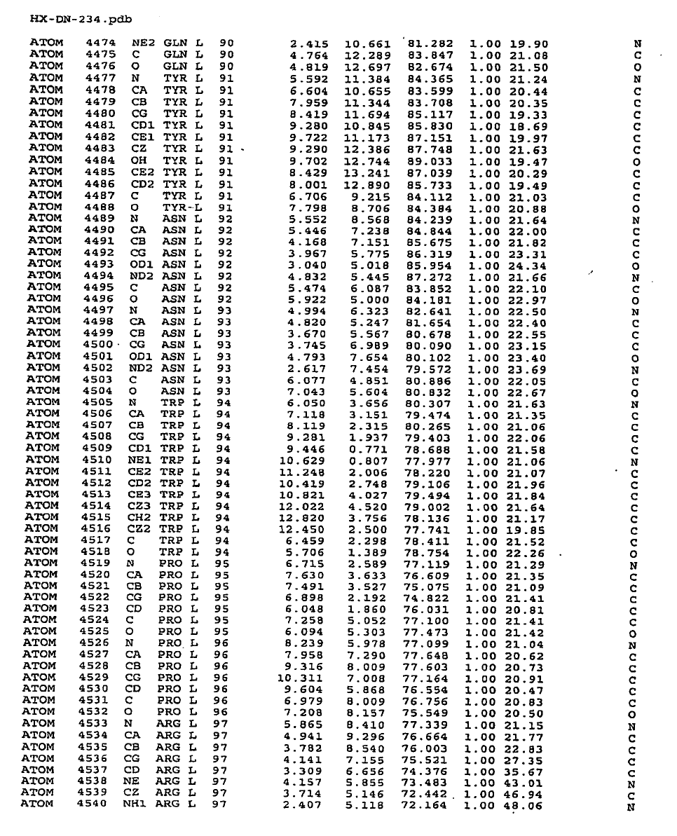

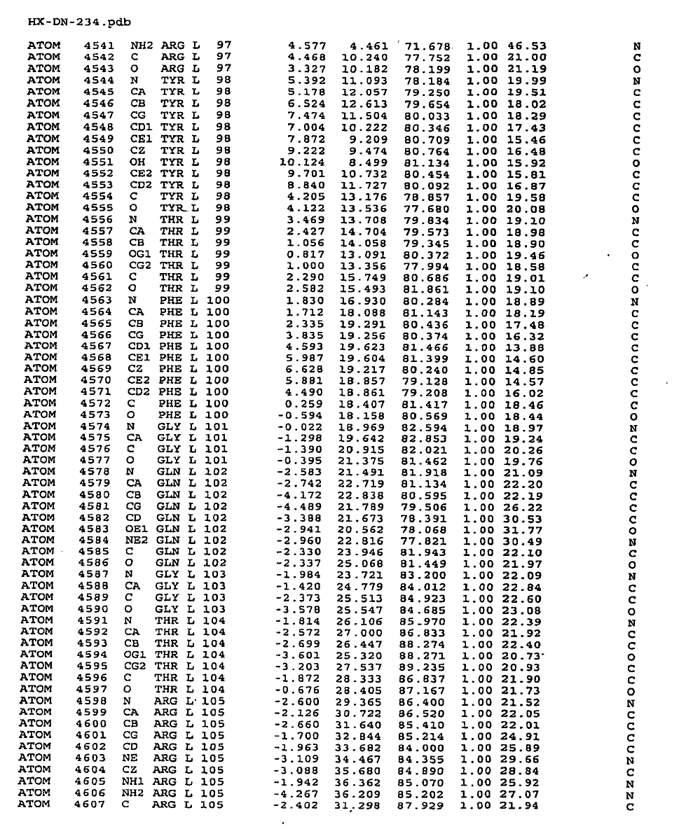

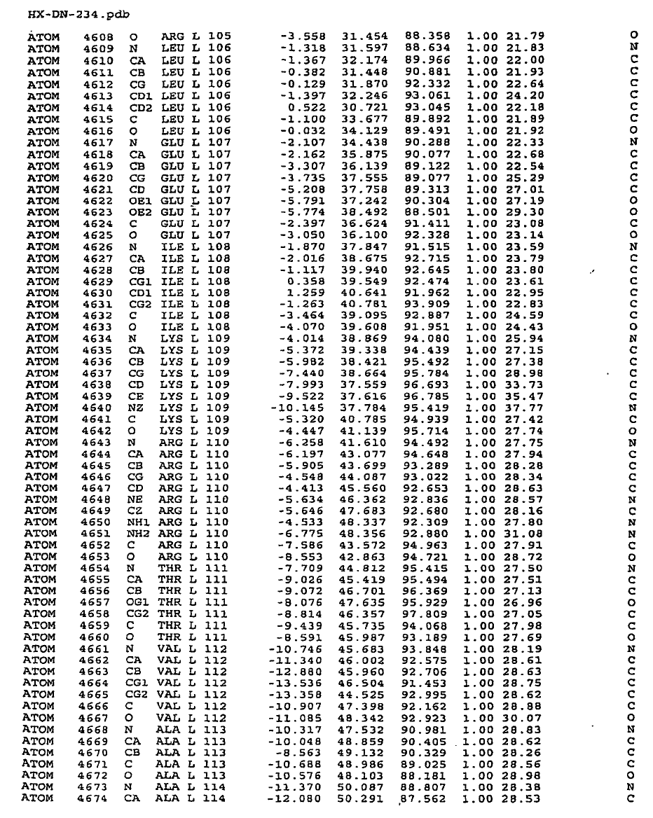

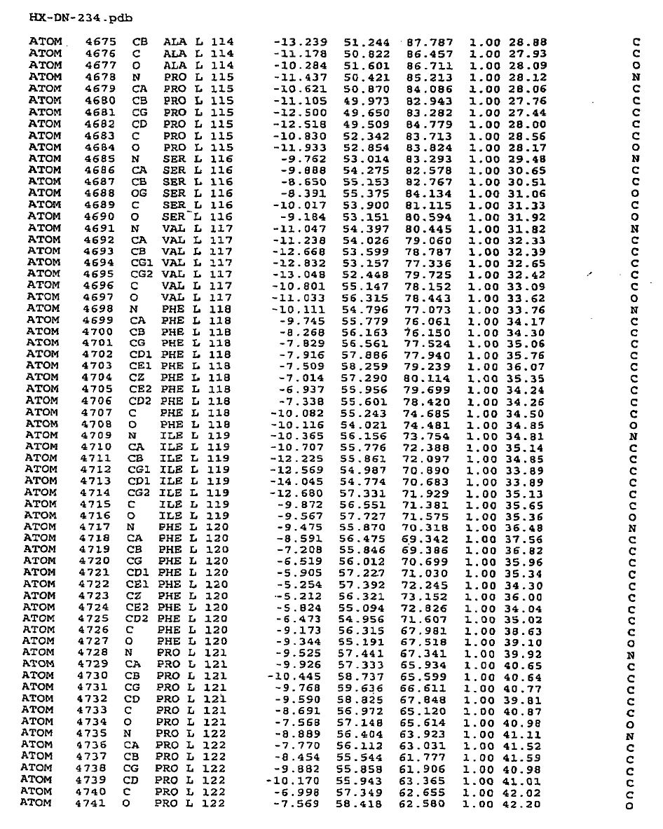

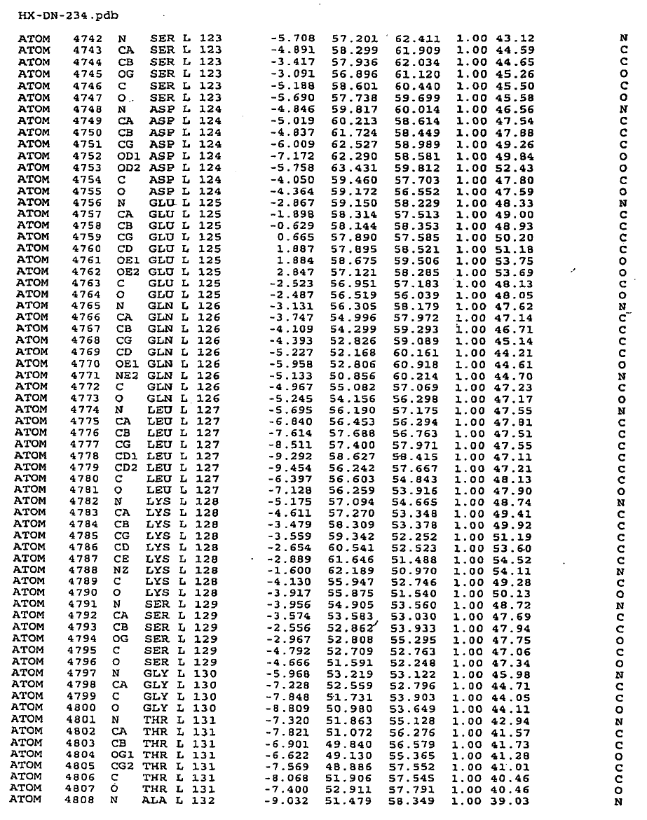

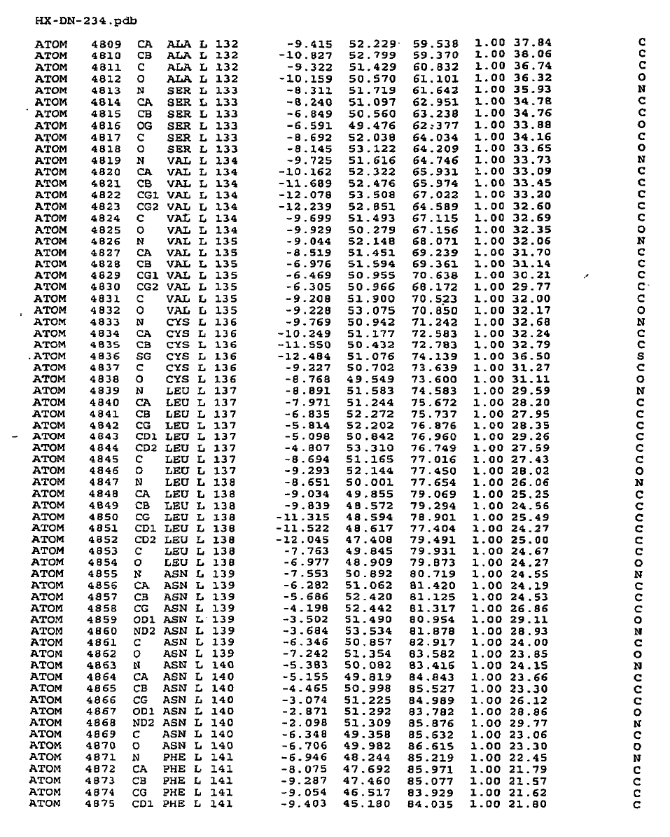

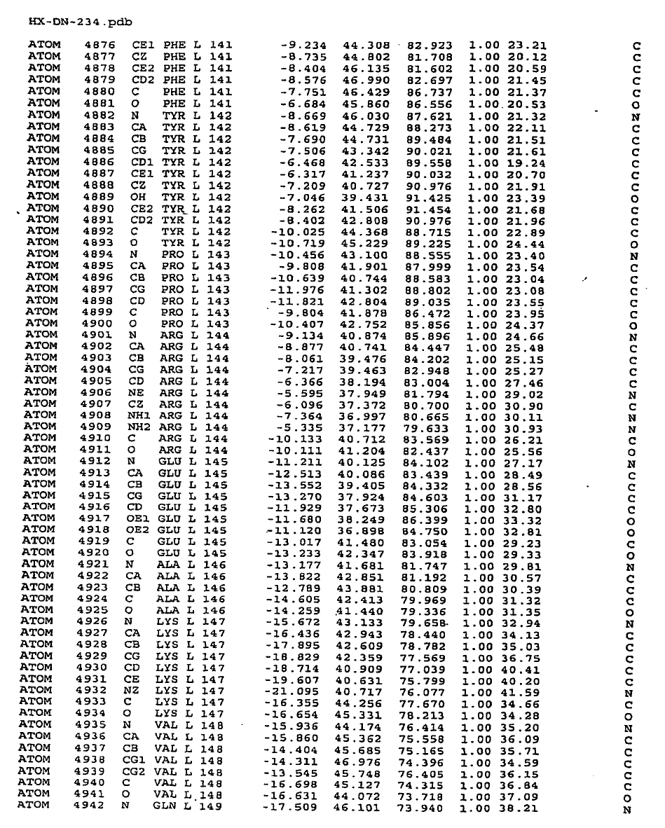

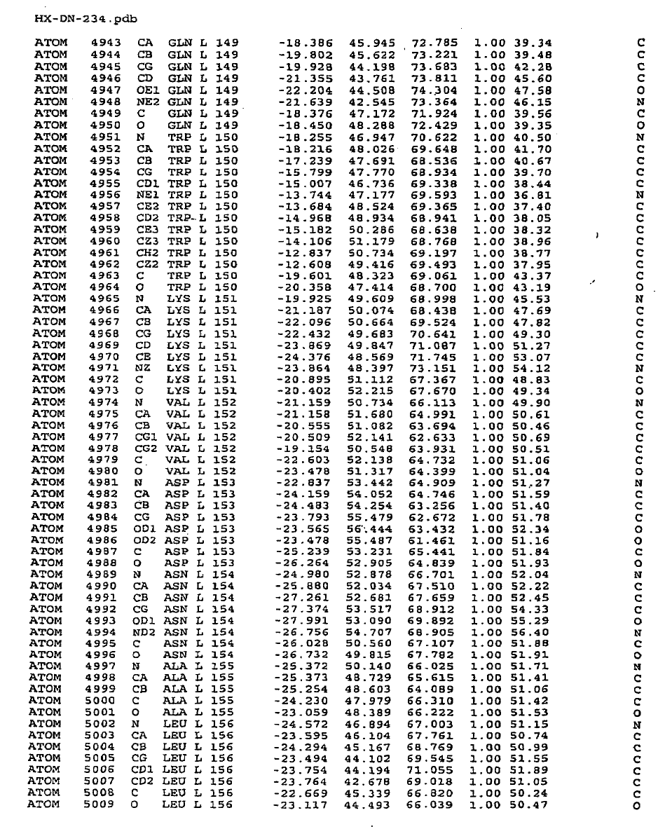

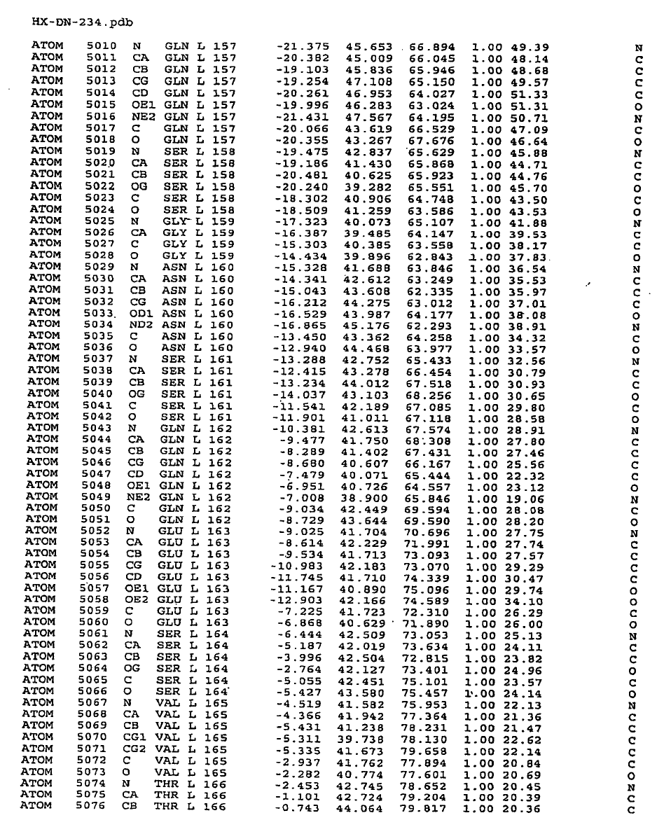

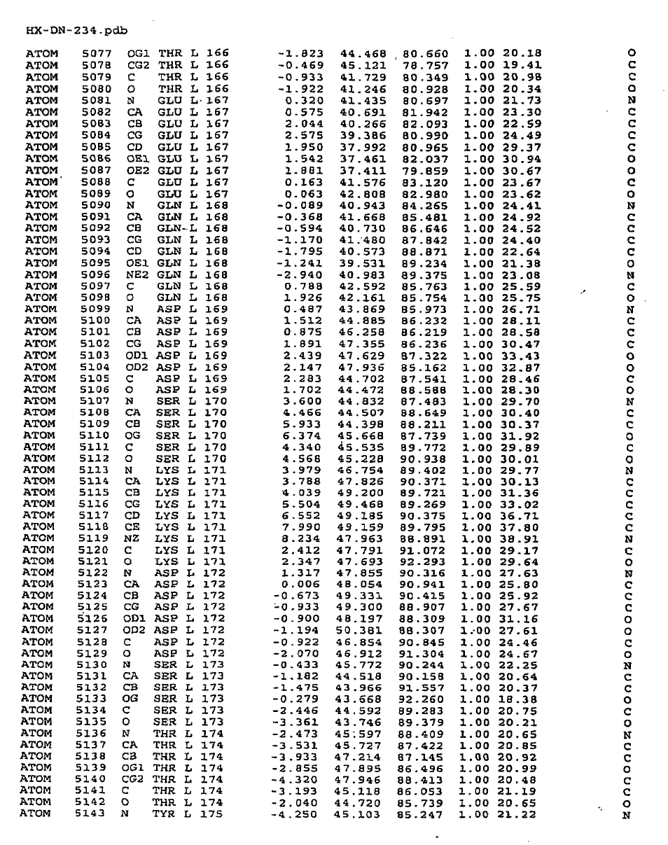

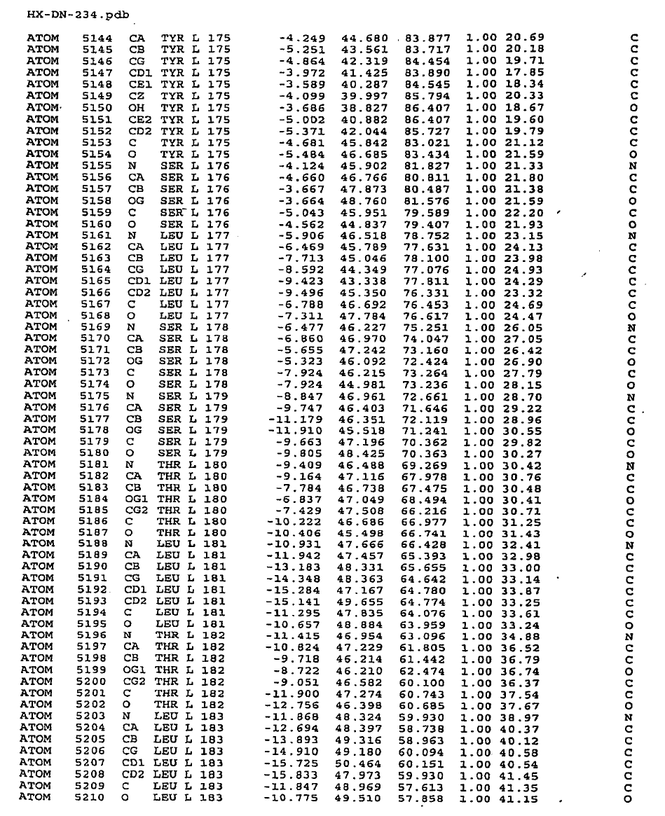

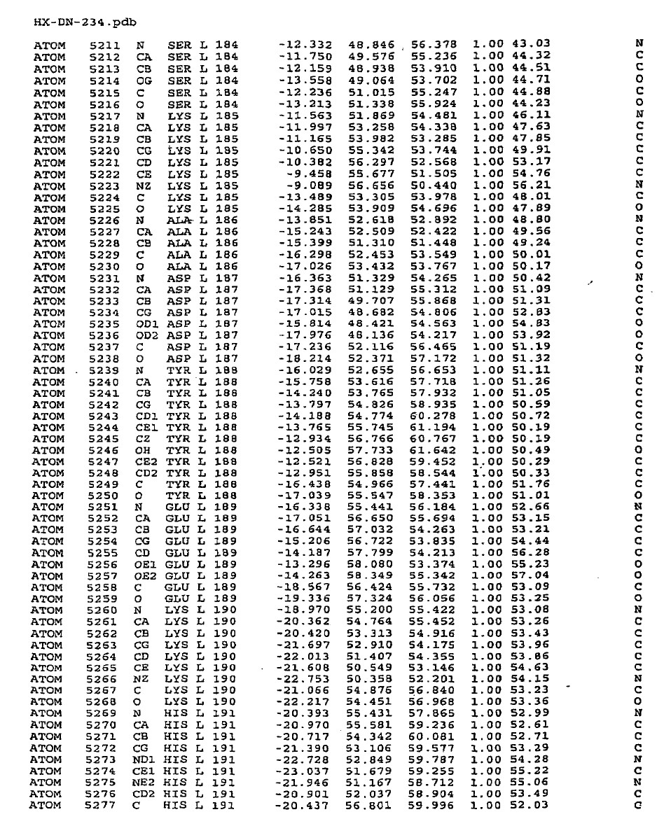

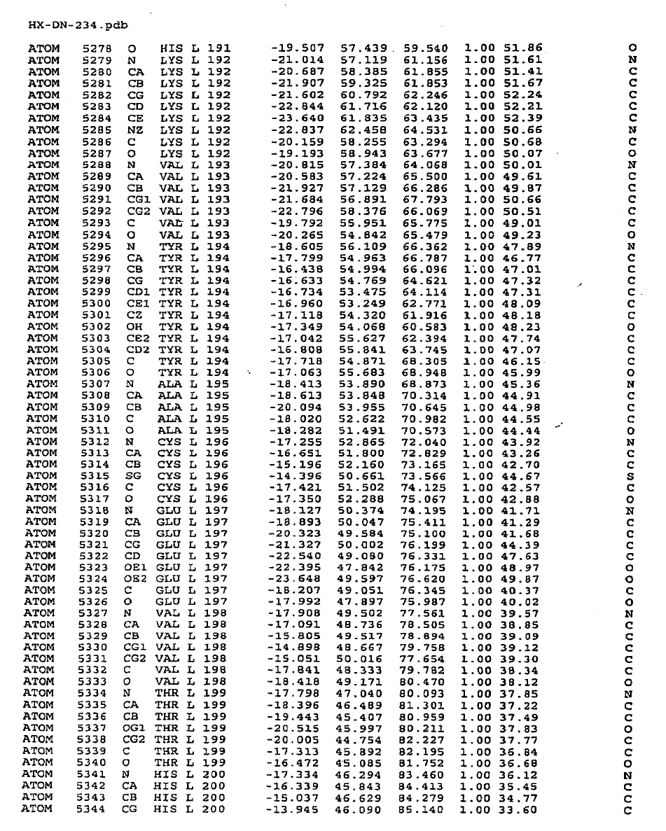

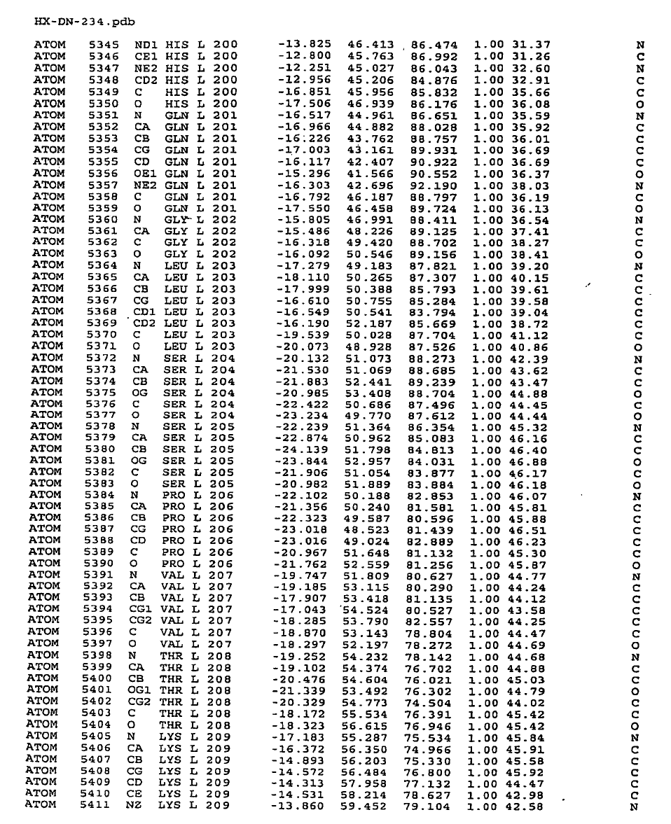

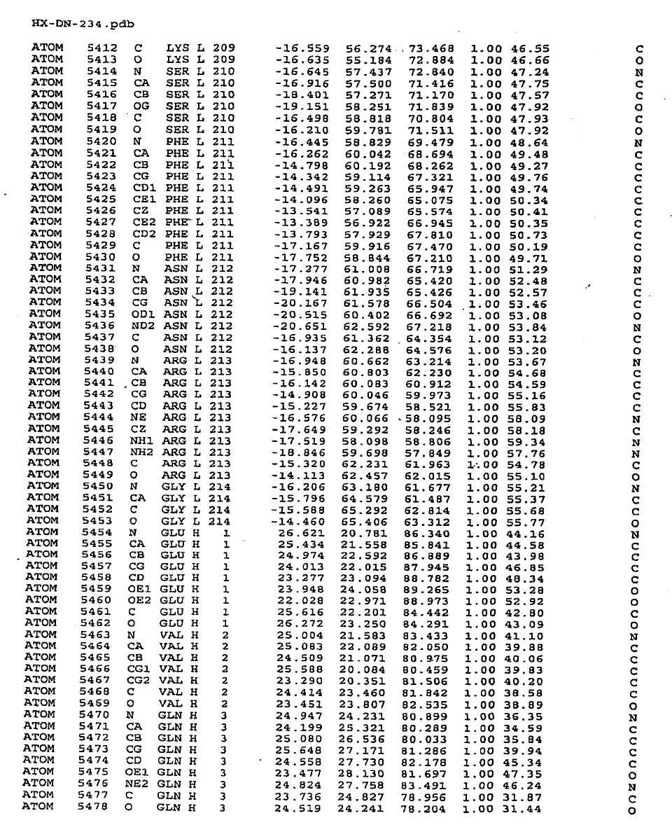

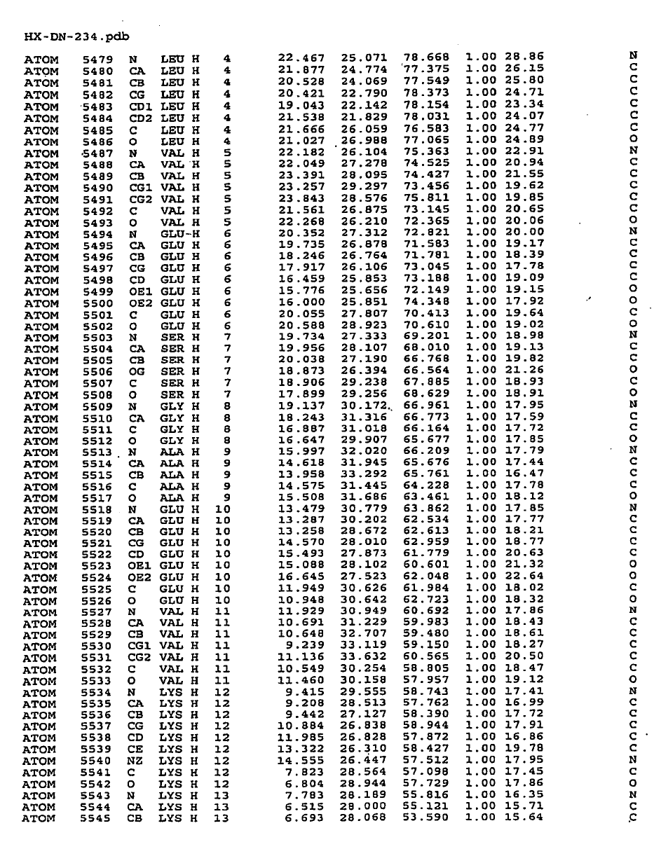

All four derivatized HX complexes were crystallized isomorphously with the original ternary complex composed of gp120, 17b Fab and D1D2 (HX-WT) (Kwong et al. 1998; Kwong et al. 2000) in the same space group P222₁ and with very similar unit cell dimensions (Table 3.5). The crystallization solutions for all four complexes were similar to that for HX-WT complex but seeding technique was indispensable for obtaining any crystal with decent diffraction quality (see Materials and Methods). Diffraction data were collected for HX-SNS-10 (FIG. 17), HX-SNS-14 (FIG. 18), HX-SNS-40 (FIG. 19), and HX-DN-234 (FIG. 20) to minimum Bragg spacings of 3.0, 2.8, 2.9 and 2.6 Å respectively (FIG. 21). Structures of these complexes were solved by rigid-body refinement of isomorphously placed components and the compounds covalently linked to Cys43 (abbreviated as compounds for Phe43 cavity) were built unambiguously into the cavity (Materials and Methods and FIG. 22). All compounds fitted into electron density very well (FIG. 22) except for the top ring of the compound in HX-DN-234, consistent with relatively high B-factors for the atoms in this top ring.

Except for the addition of the extra compounds in the Phe43 cavity introduced through modification of Cys43 of D1D2, the final models of all four structures contain essentially the same residues as those in HX-WT structure. Due to resolution limits, a few disordered residues, mostly in loop regions of gp120 lacking interpretable electron densities, were not built in the four new structures. Similarly, fewer solvent molecules were built in the derivatized complexes compared to HX-WT. As expected, the overall domain structures of all four derivatized HX complexes are similar to that of HX-WT. The introduction of the compounds to the Phe43 cavity in the derivatized HX complexes, however, does result in noticeable conformational changes in gp120 (FIG. 21 and chart below).

Comparison of complexes

| HXBc2 | | | IC₅₀ of | | Volume of |
|---|---|---|---|---|---|
| gp120:17b:CD4- | | | CD4- | | Phe43 |
| derivative | CD4- | | derivative | dₘᵢₙ | cavity |
| complexes | derivative | Structure of R | (nM) ^{$} | (Å) | (Å³) |
| HX-WT | D1D2 | 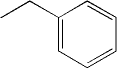 | 7.31 | 2.2^{#} | 175.2^{#} |
| HX-SNS-10 | D1D2F43C- SNS-10 | 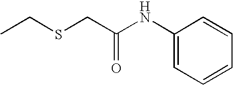 | 7.76 | 3.0 | 174.8 |
| HX-SNS-14 | D1D2F43C- SNS-14 | 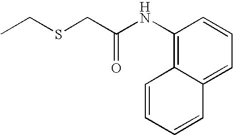 | 10.4 | 2.8 | 230.3 |
| HX-SNS-40 | D1D2F43C- SNS-40 | 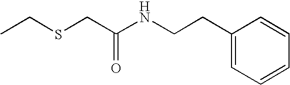 | 10.6 | 2.9 | 196.4 |
| HX-DN-234 | D1D2F43C- DN-234 | 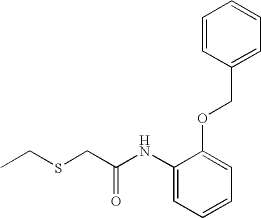 | 8.87 | 2.6 | 263.8 |

^{#}PDB-ID: 1RZJ
^{$} IC₅₀ in blocking gp120-CD4 binding in a competition ELISA (See Example II).

[symbol]

Interaction of the Phe43 Cavity with Derivatized D1D2

Unlike Phe43 in the HX-WT complex, all extensions from F43C residue in the derivatized D1D2F43C protrude into Phe43 cavity and make extensive interactions with cavity-lining residues (FIG. 22, Table 3.1). When the structures of derivatized HX complexes were superimposed using invariant regions of gp120 (see below), two distinct modes of binding to the cavity were observed for the four D1D2 derivatives distinguished primarily by on the positions of acetamide moieties in the compounds (FIG. 23A). The acetamide moieties of both D1D2F43C-SNS-10 and D1D2F43C-SNS-40 (mode I) bind at the entrance of the cavity at similar positions and make strong hydrogen-bonds (2.9 Å in both cases) with gp120 via interaction between the nitrogen atom of the acetamide group and the carbonyl oxygen of residue Asn425 in gp120. These hydrogen-bonds are reminiscent of a weak CH . . . O hydrogen-bond (3.2 Å) between Phe43 and Asn425 seen in HX-WT complex but are much stronger in terms of both hydrogen-bond distances and the atoms participating the bond. In contrast, the acetamide groups of D1D2F43C-SNS-14 and D1D2F43C-DN-234 (mode II) in the complexes are pushed away from making any appreciable interactions with N425 possibly by their much larger modifications on acetamide group than that in D1D2F43C-SNS-10 and D1D2F43C-SNS-40.

In addition to the same placement of the acetamide groups in the cavity for the CD4 derivatives in the same binding mode, the sulfur atoms of Cys43 and the chemical groups that are linked to the acetamide groups and extend further into the cavity (e.g. phenyl group for D1D2F43C-SNS-10) are also positioned similarly in the cavity (FIG. 23A). When superimposed, the Cβ and sulfur atoms of Cys43 in D1D2F43C-SNS-10 and D1D2F43C-SNS-40 are closer to the positions of the Cβ and Cγ atoms of Phe43 in D102 than those in D1D2F43C-SNS-14 and D1D2F43C-DN-234.

The CD4 mimetic protein, CD4M33, interacts with the Phe43 cavity in gp120 in a mode highly similar to that of D1D2F43C-SNS-10. The upper phenyl ring of residue 33 in CD4M33 superimposes very well with the phenyl ring in D1D2F43C-SNS-10. Although CD4M33 contains no acetamide group for hydrogen-bonding with gp120, its lower phenyl group binds to similar position on gp120 as the acetamide groups for D1D2F43C-SNS-10 and D1D2F43C-SNS-40 (FIG. 24B).

Besides the hydrogen bonds mentioned above, the carbonyl oxygen of DN-234 (the compound name is used for referring to the corresponding chemical group that is attached to Cys43 through modification of Cys43 by this compound) also makes a hydrogen bond with a water molecule, HOH47 in the HX-DN-234 structure. HOH47 is also coordinated with Gly473 of gp120 and Cys43 of D1D2F43C, at the same time (FIG. 23A). The angles of the three hydrogen bonds made by HOH47 suggest that except for the bond to Gly473, the other two are partially disordered. Furthermore, a few CH . . . O hydrogen-bonds between aromatic carbon atoms in the compounds and carbonyl oxygen atoms of gp120 are also present in all four derivatized HX complexes. Except for the hydrophilic interactions mentioned above, rest of cavity-compound interactions are hydrophobic, consistent with the hydrophobic nature of these four compounds and the generally hydrophobic character of the cavity.

TABLE 3.1
gp120 residues contacted by the side chains of
(derivatized) residue 43 of CD4.

| | | | D1D2F43C- | | D1D2F43C- | | D1D2F43C- | | D1D2F43C-DN- | |
|---|---|---|---|---|---|---|---|---|---|---|
| gp120 | D1D2 | | SNS-10 | | SNS-14 | | SNS-40 | | 234 | |
| Residue | Main | Side | Main | Side | Main | Side | Main | Side | Main | Side |
| [symbol] | | | | | | | | * | | * |
| [symbol] | | | | | * | * | | * | * | * |
| [symbol] | | | | | * | | | | * | |
| [symbol] | | | | * | | * | | | | * |
| 368 | | * | | * | | * | | * | | * |
| [symbol] | | * | | * | | * | | * | | * |
| 371 | | * | | | | * | | | | * |
| [symbol] | | | | * | * | | | * | | * |
| [symbol] | | | | | * | | * | | * | |
| [symbol] | | | | | | | | | * | &* |
| [symbol] | | | * | | * | | * | | * | |
| [symbol] | | | | | | | | * | | |
| [symbol] | | | | * | | | | * | | |
| [symbol] | &* | * | &* | | &* | * | &* | | &* | * |
| 426 | &* | | * | | * | | * | | * | |
| [symbol] | * | * | * | * | | * | * | * | * | * |
| 473 | * | | * | | * | | * | | &* | |
| [symbol] | | | | * | | * | | * | | * |

“*” indicates that the main chain (Main) and/or side chain (Side) atoms of gp120 residues interact (within 4 Å between non-hydrogen atoms) with (derivatized) D1D2.
Hydrogen bonds with bond distance equal or less than 3.5 Å between the donor and acceptor are highlighted with “&”.
gp120 residues that line the Phe43 cavity in HX-WT are indicated in bold italics.

With Phe43 residue replaced by the modified cysteines, all seven gp120 residues that interacts with side chain of Phe43 in HX-WT complex are now in contact (defined by non-hydrogen interatomic distance less than 4 Å) with the modified cysteines from four derivatized CD4 (Table 3.1 and FIG. 24A). These Phe43-interacting residues are generally located around the entrance region to Phe43 cavity and only three of them (Glu370, Asn425, and Trp427) line the inner Phe43 cavity space defined by the MS program (Connolly 1993) using a 1.4 Å probe. With derivatized CD4, much larger fractions of cavity surface are explored. The locations of these newly contacted cavity residues by the derivatized CD4 proteins correlate with their binding modes in the cavity. Side chains of F43C-SNS-10 and F43C-SNS-40 make new contacts with gp120 residues all over the cavity due to their centered location in the cavity. In contrast, most of new interactions between cavity and side chains of F43C-SNS-14/F43C-DN-234 are on the right side (e.g. Residue 256, 267, and 475) (see FIG. 23 for orientation) of the cavity where the lower phenyl rings of these two compounds bind, and near the cavity entrance where the unique positions of sulfur atoms and S—C bonds result in new interactions of gp120 (atom C of residue 473) and derivatized CD4 (FIG. 24A). In addition, F43C-SNS-40 and F43C-DN-234 also extend further into the cavity and interact with residues on the cavity ceiling (e.g. residue 377 and 112). Overall, side chains of F43C-SNS-10, F43C-SNS-14, F43C-SNS-40, and F43C-DN-234 contact 5, 7, 8, and 9 new residues in gp120 respectively in addition to 7 gp120 residues interacting with the side chain of wild type Phe43. Using the same analysis, side chain of position 33 of CD4M33 only makes new contacts with 3 additional gp120 residues and does not contact residue 368, one of 7 gp120 residues interacting with Phe43.

As the result of extensive interactions between the cavity and different CD4 derivatives, enormous changes in the shape and volume of the Phe43 cavity take place. Except for gp120 bound to D1D2F43C-SNS-10 (abbreviated as gp120_{SNS-10}, the Phe43 cavities in gp120_{SNS-14}, gp120_{SNS-40}, and gp120_{DN-234} are enlarged substantially compared to that in gp120 bound to wild type D1D2 (gp120_{D1D2}). A positive linear correlation is be found between the size of the Phe43 cavities and the molecule weight of the chemical entities residing in the cavities for all four complexes of gp120 and derivatized D1D2 (FIG. 24B). Similar to D1D2F43C-SNS-10, the volume of the cavity in gp120 bound to CD4M33 (gp120_{M33}) does not increase much compared that of gp120_{D1D2} either (FIG. 24A).

Among the cavities in these gp120 proteins bound to the derivatized CD4, the cavity in gp120_{DN-234} is the largest, showing an increase of 50% in cavity volume (calculated by removing all compounds in the cavity; see Materials and Methods) over that of gp120_{D1D2} (FIG. 24A). In fact, most of the cavity surface area not contacted by DN-234 in gp120_{DN-234} (FIG. 24A) does not exist in gp120_{D1D2} at all. Expansions of the Phe43 cavity upon binding of the compounds happen mostly at the ceiling (for gp120_{SNS-14}, gp120_{SNS-40}, and gp120_{DN-234}) and at the regions on the right side of the cavity (for gp120_{SNS-14}, and gp120_{DN-234}). Enlargement of the ceiling region of Phe43 cavity primarily involves increased cavity-exposing surface areas of the same residues (residue 276, 377, 384 and 424 in the case of gp120_{DN-234}) that line the cavity in gp120_{D1D2}. For the right-side region of the cavity, exposure of new residues (residue 473-474 and 478 in the case of gp120_{DN-234}) to the cavity occurs, partly because the side chains of Met475 in both gp120_{SNS-14} and gp120_{DN-234} flip away from the cavity (FIG. 28D), leading to further volume increase of this binding pocket. A water channel connecting the Phe43 cavity to outside of gp120 through bridging sheet is also enlarged when gp120 is bound to derivatized D1D2. Again, the enlargement is most obvious in gp120 bound to D1D2F43C-DN-234 (FIG. 24C).

The shape of the Phe43 cavity bound to the derivatives is also modified and displays high shape complementarity to the compound that binds into the cavity (FIG. 25). The entrance to the cavity becomes even narrower in gp120_{SNS-10} and gp120_{SNS-40} than that in gp120_{D1D2} possibly due to tightening effect from the hydrogen bonds between acetamide nitrogen in SNS-10/SNS-40 and the carbonyl oxygen of Asn425 at cavity entrance. Although the volume of the cavity in gp120_{SNS-10} is comparable to that in gp120_{D1D2}, the main body of the cavity changes from round-shaped in gp120_{D1D2} to heart-shaped in gp120_{SNS-10} when viewed from an angle parallel to the plane of SNS-10 (FIG. 25B). In the gp120 bound to either D1D2F43C-DN-234 or D1D2F43C-SNS-14, the cavity entrance becomes wider as Met475 adopts an alternative rotamer. In gp120_{DN-234}, the shape of the cavity is changed to be very similar to that of DN-234, leaving only a little unoccupied space in the cavity (FIG. 25).

Plasticity of gp120 and Identification of Highly Flexible Regions of gp120

The plasticity of gp120 displayed in its adapting the size and the shape of the Phe43 cavity to different compounds motivated us for further characterization of the critical residues for gp120's plasticity and identification of all flexible regions in gp120 that may not be restricted to the immediate vicinity of the cavity. Superimposition of gp120 bound to D1D2 and D1D2 derivatives revealed mostly main chain movements (along with corresponding side chain movements) but not rotamer change in the side chains in multiple regions in gp120 that are not necessarily close to the cavity (FIG. 26). These regions include α1, β16, β20, β22, LB, LF, and the loop between β3 and β4 in gp120 defined as for the initial structure (Kwong et al. 1998). In addition, significant changes are also found in variable loops and in both N- and C-termini of gp120. Only two gp120 residues around the cavity region, Met475 and Phe382, undergo significant side chain movements without changing their main chain positions too much (FIGS. 28C-D). Met475, as mentioned earlier, adopts a different rotameric conformation in gp120 bound to derivatives (D1D2F43C-SNS-14 and D1D2F43C-DN-234) in mode II. Phe382, located at backside of the cavity ceiling, swings its side chain away from the cavity in binding the derivatives (D1D2F43C-SNS-40 and D1D2F43C-DN-234) that protrude deeper into the cavity. Interestingly, residue Trp427 in β20 displays movement in both main chain and side chain atoms yet in different directions. The main chain atoms of β20 are drawn closer to the cavity because the newly formed hydrogen bond between carbonyl O of Asn425 and acetamide N in all derivatives whereas the side chain atoms of Trp427 moves away from the cavity for avoiding the steric repulsive interaction with the ligands in the cavity.

The main chain movements between gp120 molecules bound to differently derivatized D1D2, although obvious by visual inspection, are not large (0.5-2 Å). Some of the gp120 regions that have the largest movements are actually located in intrinsically flexible regions of gp120, such as variable loops. Structures of HX-WT complex and derivatized HX complexes also have different coordinate accuracy, which should be taken into account when comparing these structures. Therefore, we chose to use an objective method, namely error-scaled difference-distance matrices (Schneider 2000; Schneider 2002; Schneider 2004), for accurate structure comparison of these HX complexes.

The difference-distance matrices calculate the difference between the distance between the Cα atoms of one pair of residues in a structure and that of the corresponding pair in another structure. This distance difference is independent of the alignment of the structures of the interest. In the error-scaled difference-distance matrices, the elements of the matrices are further normalized based on the estimated errors (σ) of the coordinate precision for each structure and individual atom, allowing unbiased study of structural similarity and difference between related structures (Schneider 2000).

In our analysis, the program ESCET was used for calculation of the error-scaled distance-difference matrices of gp120 models, extracted from differently liganded-tertiary complexes, using a 1.3σ cutoff (FIG. 27). The 1.3σ cutoff is big enough for estimating the coordinate errors originated from the lattice variance yet small enough to be sensitive to the real coordinate differences. As control experiments for justifying the use of 1.3σ as proper cutoff for ESCET program, we calculated the error-scaled difference-distance matrices for each pair of non-crystallographic symmetry (NCS)-related gp120 molecules found in YU-M33 or YU-F23 complexes (Huang et al. 2005). The percentages of the matrices' elements bigger than 1.3σ were found to be 0% and 2% for the pair of gp120 molecules in YU-M33 and YU-F23 respectively, indicating gp120_{M33} and gp120_{F23} are identical by ESCET standard (less than 2% of matrix elements are bigger than 1.3σ). The fact that NCS-related models of same gp120 were found to be invariant in our analysis suggests that 1.3σ is a good estimation for the coordinate errors caused by lattice variance.

In the calculation of difference matrices of different gp120 models, the gp120 residues were also distance-sorted based on the ascending distance of the Cα of each residue to the center of the Phe43 cavity defined by the position equivalent to that of atom C4 of the phenyl ring of residue 43 in D1D2F43C-SNS-10. Inspection of all pairwise comparisons between different gp120 models revealed that gp120_{D1D2} was not considered to be identical to any of the gp120 models bound to derivatized D1D2, whereas all four gp120 structures complexed with derivatized D1D2 were considered to be the same (Table 3.2). Consistent with the larger sizes of Phe43 cavities in gp120_{SNS-14} and gp120_{DN-234}, more structural differences were noted in difference matrices between gp120_{D1D2} and these two gp120 models compared to the difference between gp120_{D1D2} and gp120_{SNS-10}/gp120_{SNS-40}. Based on the distance-sorted error-scaled difference-distance matrices (FIG. 27), the first 120 gp120 residues, whose Cα atoms are within 18 Å of the Phe43 cavity, contributed the majority of the elements bigger than 1.3σ in the matrices. In the error-scaled difference-distance matrices of these 120 residues of different gp120 models, the percentages of matrix elements of gp120_{D1D2} and gp120_{Derivatized-D1D2} bigger than 1.3σ were found to be 4.8% to 13.4%, compared to only 0%-2.5% between any pair of different gp120_{Derivatized-D1D2} (Table 3.2). As expected, gp120_{DN-234} among all gp120_{Derivatized-D1D2} models showed largest difference to gp120_{D1D2} in the difference matrix analysis.

TABLE 3.2
Pair-wise comparisons of gp120 structures by error-
scaled difference-distance matrices.

| | gp120_{D1D2} | gp120_{SNS-10} | gp120_{SNS-14} | gp120_{SNS-40} | gp120_{DN-234} |
|---|---|---|---|---|---|
| gp120_{D1D2} | | 2.5 | 4.0 | 2.3 | 4.8 |
| gp120_{SNS-10} | 4.8 | | 0.7 | 0.3 | 1.0 |
| gp120_{SNS-14} | 7.8 | 1.2 | | 1.6 | 1.4 |
| gp120_{SNS-40} | 6.0 | 0.0 | 1.2 | | 0.7 |
| gp120_{DN-234} | 13.4 | 2.0 | 2.5 | 0.7 | |

The differences are represented by the percentages of elements bigger than 1.3σ. Those on upper right triangle of the table are based on Cα atoms of all 293 residues; those on lower left triangle are based on the Cα atoms of 120 residues that are within 18 Å of the cavity.

All D1D2 derivatives bind gp120 in similar ways and introduce similar conformational changes in gp120 (FIG. 26). Because biggest changes are found in gp120 bound to D1D2F43C-DN-234, we then focused on the structural comparison between gp120_{D1D2} and gp120_{DN-234} to study gp120's plasticity.

These two gp120 models were subjected to rigid-body analysis in the ESCET program with the following parameters: n_{hyp}=20, wₚ=20.0, ε₁=1.3, εₕ=4, rₘᵤₜ=5.0%. Out of 293 gp120 residues common to both gp120_{D1D2} and gp120_{DN-234}, 255 residues (85-105, 118-208, 214-248, 254-375, 378-397, 412-420, 431-443, and 446-491) were identified as invariant region. The remaining 38 residues of gp120 (106-117, 209-213, 249-253, 376-377, 410-411, 421-430, and 444-445) were identified as flexible. These flexible residues of gp120 are located to α1, loop between β3 and β4, β8, β16, V4 loop, β20, and β22 respectively (FIG. 28A). These regions are consistent with the regions that show significant main chain movements when comparing gp120_{D1D2} and gp120_{Derivatized-D1D2} (FIG. 26) except the flexible loops (LB, LF) are no longer identified as flexible whereas some of regions showing only a little main chain movements (β8) are identified as flexible in ESCET analysis, in which coordinate errors are considered. As expected by definition, RMS deviations of the flexible residues between five gp120 structures are much higher (0.8-1.3 Å) than that of all gp120 residues (0.4-0.6 Å) (Table 3.3). On the other hand, all the secondary structural elements identified as flexible for these derivatives were shown to be structurally invariant when comparing non-crystallographic symmetry-related gp120 molecules found in both YU-M33 and YU-F23 using same ESCET rigid body analysis (data not shown). Similar results were obtained, except for the loop between β3 and β4, when comparing gp120 from different strains (including HXBc2, YU2 and JR-FL) bound to wild type D1D2 using same ESCET analysis (data not shown). These results again confirm that the criteria we used in ESCET analysis are sensitive in identification of flexible residues due to different ligand binding but not due to lattice variance.

TABLE 3.3
RMS deviations (Å) of C_{α} atoms of all 293 residues
(upper right triangle) or 38 flexible residues (lower left
triangle) between gp120 structures extracted from their complexes
with different D1D2-R.

| | gp120_{D1D2} | gp120_{SNS-10} | gp120_{SNS-14} | gp120_{SNS-40} | gp120_{DN-234} |
|---|---|---|---|---|---|
| gp120_{D1D2} | | 0.6 | 0.7 | 0.6 | 0.6 |
| gp120_{SNS-10} | 1.3 | | 0.4 | 0.4 | 0.4 |
| gp120_{SNS-14} | 1.4 | 0.8 | | 0.5 | 0.5 |
| gp120_{SNS-40} | 1.3 | 0.7 | 1.1 | | 0.6 |
| gp120_{DN-234} | 1.2 | 1.2 | 0.6 | 1.3 | |

All gp120 structures were superimposed using C_{α} atoms of invariant regions of gp120. Both flexible (106-117, 209-213, 249-253, 376-377, 410-411, 421-430, and 444-445) and invariant (85-105, 118-208, 214-248, 254-375, 378-397, 412-420, 431-443, and 446-491) regions were classified by ESCET.

Except for residue 410-411, which belong to intrinsically variable regions (V4 loop), the remaining 36 gp120 residues that show significant structural rearrangement upon binding D1D2F43C-DN-234 are located at conserved regions of gp120 and are mapped primarily to the inner domain and bridging sheet. With respect to gp120_{D1D2}, residues 106-117, 209-213, 376-377, and 444-445 in gp120_{DN-234} move away from the Phe43 cavity whereas residues 249-253 and 421-430 move closer to the Phe43 cavity. Most of these 36 residues, however, do not interact directly with D1D2F43C-DN-234 at all (FIG. 28B). In fact, only 9 residues out of 36 flexible gp120 residues identified by ESCET, namely residues 112, 376-377, and 425-430, directly contact D1D2F43C-DN-234 in HX-DN-234 complex. Among them, all but residue 428-430 only interact with the modified side chain of Cys43. Interestingly, three out of these nine residues are aromatic residues (Trp112, Phe376, and Trp427) and make extensive hydrophobic interactions with DN-234 moiety inside the cavity and other gp120 residues around the cavity that are identified as flexible regions (FIG. 28 C-D). These CD4-contacting gp120 residues may play a key role in propagating conformational changes from the Phe43 cavity region to other more remote areas in gp120.

CD4-gp120 Interface Other than the Phe43 Cavity

Overall features of the interface between gp120 and D1D2 derivatives other than the Phe43 cavity are the same as that of gp120-CD4. In the HX-DN-234 complex, all gp120 residues contacted by CD4 interact with D1D2F43C-DN-234, except for a residue in the V5 loop, Asn460, which is disordered and not built in gp120_{DN-234}. New interfacial gp120 residues that contact D1D2F43C-DN-234 are located mostly around the cavity region (FIG. 29A). A few new gp120 interfacial residues have been also identified outside the cavity region (FIG. 29B). These residues, however, make weak interactions (>3.8 Å) with D1D2F43C-DN-234 and these weak interactions are introduced either by slightly different rotamers built in gp120_{DN-234} residues (residue 469 and 477) with respect to gp120_{D1D2} or from gp120 residue located in variable loop region (residue 124 in V1/V2 loop).

Although most of the interactions are conserved between D1D2 and gp120, some of them especially the hydrophilic interactions between gp120 and residue 42 to 45 in CDR2 loops of D1D2 derivatives are weakened primarily due to main chain movements of CDR2 loops in D1D2 derivatives and to less extent caused by that of β15 in gp120. In the HX-DN-234 complex, these main chain movements of both D1D2F43C-DN-234 (average 0.48 Å Cα movements in residue 42-45) and gp120 away from the location of Phe43 cavity result in an average increase of 0.14 Å in the length of 7 hydrogen bonds (FIG. 29C) between gp120 and residues 42-45 and 59 of D1D2F43C-DN-234 with respect to the corresponding hydrogen bond lengths in HX-WT complex. Among these 7 hydrogen bonds, the one between atom OG of Ser42 in D1D2F43C-DN-234 and atom O of Trp427 in gp120 is the most weakened (increase from 3.09 Å to 3.65 Å). The hydrogen bond partner of residue Ser365 of gp120 is also observed to change from Pro48 of D1D2 in the HX-WT complex to Lys46 in the HX-DN-234 complex due to different rotamer built for Ser365 in two structures. Because the electron density for side chain of Ser365 supports both rotamers, this change of gp120-CD4 interaction may not be relevant to the interaction of derivatized D1D2 with the Phe43 cavity of gp120.

gp120-17b Interactions in the Presence of Derivatized CD4

Although some residues at the gp120-binding site were identified as structurally variant regions by ESCET when comparing structures of gp120-bound 17b in the presence of either D1D2 or D1D2F43C-DN-234, most of gp120-17b interactions are unchanged. Interestingly, many 17b-interacting gp120 residues are located either close to or belong to the regions that show substantial movement upon D1D2F43C-DN-234 binding (FIG. 30).

Thermodynamic Analysis of the Binding of Derivatized D1D2 and 17b to gp120

The crystal structures of gp120 complexed with derivatized D1D2 revealed that core gp120 undergoes structural rearrangement upon binding to derivatized D1D2. To extend our understanding of the biological relevance of these conformational changes in gp120, we further studied the thermodynamics of the binding of gp120 to derivatized D1D2 and the binding of gp120 to 17b antibody in the presence of saturating concentrations of derivatized D1D2 using isothermal titration calorimetric experiments. Wild type D1D2 and two D1D2 derivatives, D1D2F43C-SNS-10 and D1D2F43C-DN-234, which binding the Phe43 cavity of gp120 in different modes, were used in the thermodynamic study.

Direct Binding of Wild-Type and Derivatized D1D2 to gp120

As reported earlier (Myszka et al. 2000; Leavitt et al. 2004), D1D2 binds gp120 with an unusually large and favorable enthalpy change, ΔH, and a large unfavorable entropy term, −TΔS (Table 3.4). In comparison, both D1D2 derivatives, especially D1D2F43C-DN-234, bind gp120 with smaller values for the favorable ΔH and the unfavorable −T□S compared to that of wild-type D1D2. The measured Kd values for the binding of D1D2 or D1D2 derivatives to gp120 agree well with IC₅₀ values reported in Example II: D1D2F43C-SNS-10 binds gp120 with similar affinity as that for D1D2 whereas D1D2F43C-DN-234 binds gp120 with less affinity.

The temperature dependence of the enthalpy change, i.e. the change in heat capacity ΔCₚ, for direct binding to gp120 is significantly different for the wild-type D1D2 compared to the two derivatized forms, D1D2F43C-SNS-10 and D1D2F43C-DN-234. Binding of D1D2 to gp120 is associated with an extremely large negative change in heat capacity of −1800 cal/(K×mol), a value similar to that obtained for protein folding. ΔCₚ for binding of gp120 to D1D2F43C-SNS-10 and D1D2F43C-DN-234, however, are 22% and 33% less than that for binding of gp120 to D1D2, valued at −1400 and −1200 cal/(K×mol) respectively (Table 3.4).

TABLE 3.4
Binding thermodynamics of wild-type and derivatized
D1D2 to YU2 gp120*.

| | | | | | | ΔCp |
|---|---|---|---|---|---|---|
| D1D2- | IC₅₀ | K_{d} | ΔG | ΔH | −TΔS | (cal K⁻¹ |
| derivative | (nM) | (nM) | (kcal/mol) | (kcal/mol) | (kcal/mol) | mol⁻¹⁾ |
| D1D2 | 7.31 ± 1.07 | 23 ± 3 | −10.5 ± 0.1 | −34.6 ± 0.4 | 24.1 ± 0.5 | −1800 ± 100 |
| D1D2F4 | 7.76 ± 0.83 | 23 ± 3 | −10.5 ± 0.1 | −33.8 ± 0.5 | 23.3 ± 0.6 | −1400 ± 200 |
| 3C- | | | | | | |
| SNS-10 | | | | | | |
| D1D2F4 | 8.87 ± 1.09 | 45 ± 5 | −10.0 ± 0.1 | −30.1 ± 0.4 | 20.1 ± 0.5 | −1200 ± 100 |
| 3C- | | | | | | |
| DN-234 | | | | | | |

*The values for the affinity (K_{d} and ΔG), ΔH and −TΔS were determined at 25° C. The changes in heat capacity were determined from titrations performed at different temperatures. IC₅₀ values (see Example II) are also listed for comparison.

A binding reaction associated with large favorable enthalpy and large unfavorable entropy changes together with a large negative change in heat capacity is characteristic of a process that involves large conformational changes. These changes in entropy and heat capacity can be analyzed as the equivalent number of unfolded residues that become conformationally restricted upon complexation (Luque et al. 1998). Such an analysis of the values presented here shows that binding of wild-type D1D2 to gp120 generates an ordering equivalent of about 120 residues whereas D1D2F43C-SNS-10 structures about 90 and D1D2F43C-DN-234 only 80 residues.

Enhancement of gp120 Binding to the Co-Receptor Site

Binding of CD4 to gp120 leads to the formation of chemokine-receptor binding site on gp120 (Trkola et al. 1996; Wu et al. 1996). The binding of derivatized D1D2-bound gp120 to 17b, a gp120 antibody that recognizes the chemokine-receptor site, is used to assess the formation of co-receptor site on gp120 upon derivative binding. It has been observed that the binding of 17b to gp120, especially to core gp120, can be greatly enhanced in the presence of CD4 (Kwong et al. 2002; Huang et al. 2005). Here, we found that in the presence of D1D2, the binding affinity of 17b to YU2 gp120 is enhanced by 4 fold, characterized by a 0.8 kcal/mol. increase in AG value (FIG. 31). The enhancement is also seen with the two derivatives; however, the effects are 25% and 38% less for D1D2F43C-SNS-10 and D1D2F43C-DN-234 respectively.

### Discussion

### Correlations Between SAR and Structural Studies

Structural characterization of the Phe43 cavity as a binding site for diverse ligands, in the context of SAR study (Example II), provides us with a comprehensive understanding on the molecular details of how gp120 recognizes the cavity-targeting ligands. The four D1D2 derivatives, whose structures are presented here, bind the Phe43 cavity through two distinct modes (FIG. 23). The derivatives D1D2F43C-SNS-10 and D1D2F43C-SNS-40 with relatively small aromatic groups (phenyl and phenethyl) attached to their acetamide moieties, bind the Phe43 cavity in mode I. A hydrogen bond between the acetamide N and O atoms of Asn425 in core gp120 is involved in positioning the acetamide group and narrows the cavity entrance even further (FIG. 25B), such that any branching of the derivatives that crowds the entrance will be highly unfavorable, as observed in SAR study. The perseverance of this hydrogen bond between the D1D2 derivatives and the full-length gp120, which is used in SAR study (Example II), is evidences by three-fold reduction in gp120-binding affinity from D1D2F43C-SNS-10 to D1D2F43C-DN-180 (the acetamide N atom replaced by a C atom). Interestingly, CD4M33 also binds in a mode very similar to mode I even though no hydrogen bond is involved.

Mode II binding is found in the binding of bulkier groups, namely naphthalene and benzyloxy-phenyl groups (D1D2F43C-SNS-14 and D1D2F43C-DN-234), into the cavity, featuring i) weakening of the hydrogen bond seen in mode I and ii) the enlargement of the cavity entrance by an alternative Met475 rotamer configuration.

Different locations of the ligands in these two binding modes suggest that there are more than one optimal binding configurations in the Phe43 cavity. Mode II binding, however, is probably less favored than mode I binding not only because of the weakening of the hydrogen bond but also because the new rotameric conformation of Met375 results in its slightly repulsive interaction (3.2 Å) with Trp479. The penalty for the widening of the cavity entrance is probably responsible for the low affinity binding between gp120 and aliphatic ligands with branches that crowds the entrance (SAR study, Example II). D1D2F43C-SNS-14 and D1D2F43C-DN-234, on the other hand, may compensate the lost of affinity by engaging extensive favorable interactions within the cavity, as evidenced by the nearly perfect shape complementation of DN-234 and the cavity (FIG. 25). Yet still neither of these two derivatives binds to gp120 better than D1D2F43C-SNS-10.

D1D2F43C-DN-234, a mode II binder, has largest aryl group attached to the acetamide moiety among all high affinity D1D2 derivatives identified in Example II with IC₅₀ less than 10 nM and has the largest potential for hydrophobic interactions with the cavity. D1D2 derivatives with smaller aryl group, if binding gp120 in mode II, should have affinity to gp120 no greater than that for D1D2F43C-DN-234. Thus the best gp120-binding derivatives, e.g. D1D2F43C-SNS-12 and D1D2F43C-DN-52, whose affinities to gp120 double that for D1D2F43C-DN-234, most likely do not recognize gp120 in mode II. Furthermore, D1D2F43C-SNS-12 and D1D2F43C-DN-52 are essentially derivatized D1D2F43C-SNS-10 with the para position of the phenyl group substituted with isopropyl and nitro group respectively. Although alternative binding modes other than mode I and II may exist, carefully inspection of the interface of gp120 and D1D2F43C SNS-10 (mode I binding) suggests that the addition of either isopropyl or nitro group at the para position to SNS-10 should fit perfectly in the unoccupied space in the “two corners” of the hearted-shaped cavity (FIG. 25B) and leads to a flawless complementation between the Phe43-cavity binding site and the ligand. Thus, model I binding is highly likely utilized in the recognition of gp120 and D1D2F43C-SNS-12/D1D2F43C-DN-52.

In summary, the structural information on the binding of core gp120 and the derivatized D1D2 proteins strongly support the results of SAR study using full length gp120 and indicates that characteristics of the Phe43 cavity are conserved from core gp120 to full length gp120. These findings should also help the design of next-generation cavity ligands, in a structure-based fashion.

gp120 Plasticity

The flexibleness of the Phe43 cavity to different ligands suggests that the Phe43 cavity, like rest of D1D2 interface on gp120, binds its ligand in an induced-fit mechanism. The adaptability of gp120 arises mostly from main chain but not side chain movements. Met475 and Phe382 are the only two interface residue in gp120 that adopt different rotameric conformation in binding D1D2F43C-SNS-14 and D1D2F43C-DN-234 from that in binding D1D2.

All gp120-(derivatized D1D2) interfacial residues that belong to outer domain (altogether 23 residues), except for residues 376-377 of β16, show no significant main-chain positional adjustment compared to gp120_{D1D2} (FIG. 28). As part of cavity-lining residues, Residues 376-377 are structural neighbors to the inner domain and bridging sheet and only interact with derivatized D1D2. By contrast, 7 (residue 112 in α1, and residues 425-430 in β20) out of 11 gp120 inner-domain residues that interact with either D1D2 or derivatized D1D2 are found to be structurally flexible when comparing gp120_{D1D2} and gp120_{DN-234}.

Interestingly, main chain atoms of residues 472-475, which are located on tip of α5 (at junction between outer and inner domains) and the loop connecting α5 to β24 (outer domain), are found to be structurally rigid despite the fact the side chain of Met475 is flipped in the reconstruction of the Phe43 cavity upon binding its ligands. Although Met475 only contacts derivatized D1D2, residues 472-474 interact with wild-type D1D2 and its derivatives. In conclusion, gp120-(CD4 derivative) interfacial residues in gp120 outer domain except for those from β16 are structurally rigid, whereas most interfacial residues that are located in the inner domain or bridging sheet except for α5 have high degree of flexibility in binding cavity-filling ligands.

In addition to the above 9 D1D2-contacting gp120 residues that are flexible (“hotspot” residues), other 27 gp120 residues that do not directly contact D1D2F43C-DN234 were also identified to move significantly upon binding D1D2F43C-DN234 (FIG. 28, 27 residues do not include 410-411 of V4 loop). Again, 24 out of these 27 residues are in either the inner domain or the bridging sheet.

These results are consistent with the conservation of the outer domains between structures of free SIV gp120 and CD4-bound gp120, except for CD4-binding strand β15 (Chen et al. 2005) and its vicinity strands including β16, whose flexibility was also observed in the current study. Helix α5, especially its N-terminus, identified as rigid here, is also the only secondary structural element in the inner domain and the bridging sheet that displays very little dislocation in comparing structures of free SIV gp120 and liganded-gp120.

Pathway of Motion Propagation in gp120 Bound by Derivatized D1D2

The 27 gp120 residues that do not contact derivatized D1D2 yet display structural rearrangement, are located in 6 different segments including α1, β8, β16, β20, β22, and the loop between β3 to β4. Their motions are most likely transuded from the movements observed for derivatized D1D2-contacting residues, which include the 9 “hotspot” residues and residues Met475 and Phe382, for which the rotamer change is the primary mechanism used for binding derivatized D1D2. However, for the extremely high structural variance observed for residue 209-213 in the loop connecting β3 and β4, we could not rule out the possible contribution of the intrinsic flexibility, since this region is also identified as flexible when comparing gp120 from different HIV-1 strains (data not shown) and is completely disordered in the structure of free SIV gp120 (Chen et al. 2005).

By analyzing the inter-residue contacts between the non-CD4 contacting residues and the CD4 contacting residues, we propose here a possible pathway for the motion propagation from the Phe43 cavity to more remote areas in gp120 (FIG. 32). β20 contains six “hotspot” residues and moves close to the cavity in the presence of ligands. Trp427 in β20, however, as discussed in the Results, has its side chain group move away (˜0.5 Å) from the cavity. The movement is passed on to Ile109 and Trp112 in α1 through their tight hydrophobic interactions with Trp427. The transduced motions to Ile109 and Trp112, in combination with the movement of “hotspot” residue Trp112 directly resulted from interaction with derivatized D1D2, lead to the structural rearrangement in α1 Further interaction of Trp112 with Phe210 may in turn contribute to the high flexibility in the loop connecting β3 to β4. Motions in β16, which contains two “hotspot” residues Asn377 and Phe376, are partially caused by steric repulsion between cavity ligands and main chain atoms of Phe376. Side chain movement of Phe382 can also be passed on to β16 through hydrophobic interaction between Phe382 and Asn377. Furthermore, the disulfide linkage between Cys378 (β16) and Cys445 (β22) may help to propagate the motions from β16 to β22. No obvious connection could be found between D1D2-contacting residues and residue 249-253 (β8) (data not shown). Conformational changes seen in β8 may reflect its intrinsic flexibility, evidenced by its conversion to an α-helix in free SIV gp120.

Co-Receptor Binding and Conformational State of gp120 Bound by Derivatized D1D2

It is surprising to observe conformational changes in multiple segments in gp120_{derivatized-D1D2} compared to gp120_{D1D2}. CD4-restrained gp120 appears to still have considerable flexibility even in the regions that are directly bound by wild type D1D2. Even more surprisingly, the conformational changes can be propagated to the chemokine-receptor/17b binding sites despite the fact that 17b is also present in the determined gp120 complexes and should further rigidify gp120. The indispensable usage of the 17b antibody in X-ray crystallographic study of gp120 bound by the derivatized CD4 limits our capability to study the real conformation of gp120 bound only by the derivatized CD4, although this conformation of gp120 should not differ too much from that bound by both 17b and the derivatized CD4, based on relatively small entropy changes in the binding of derivatized CD4-bound gp120 to 17b (FIG. 31).

The bridging sheet has been found to be the binding sites for 17b (Kwong et al. 1998; Kwong et al. 2000) and presumably for the chemokine receptors. A strand in the bridging sheet, β20, moves noticeably in binding of D1D2 derivatives to gp120. In addition, conformational changes have also been located to other residues involved in chemokine-receptor binding that were identified by mutagenesis study (Rizzuto et al. 1998) including Lys177 in α1, Asn377 in β16, and Arg444 in β22. Although no significant difference was found when comparing the gp120-17b interface in the presence of D1D2 to that in the presence of D1D2 derivatives, probably due to the inclusion of 17b in the complexes for structural study, thermodynamic study revealed reduced affinity of 17b and gp120 pre-bound with derivatized D1D2 instead of D1D2 (FIG. 31).

This finding suggests that the real conformations of gp120 in solution with the Phe43 cavity bound to the derivatized D1D2 are indeed different than that of D1D2-bound gp120. The structural difference between gp120 bound to D1D2 and its derivatives in the absence of 17b may also be greater than what is observed in the crystal structures of gp120_{D1D2} and gp120_{derivatized-D1D2} solved in the presence of 17b. Smaller unfavorable entropy changes, less □Cₚ and fewer structured residues in gp120 binding to the derivatized D1D2 further support the notion that D1D2-bound gp120 adopts a much less structured state when Phe43 cavity is filled by proper ligands. This intermediate state between free and D1D2-bound gp120 may involve reorganization of identified flexible secondary structural elements in our study, especially, α1 and β20/21, implicated by the relatively easy conversion of free SIVgp120 structure to a near CD4-bound conformation through rearrangement of α1 and β20/21 (Pan et al. 2005). The degree of the reorganization in solution, however, must be greater that what is shown in the crystal structures, which are highly constrained by both lattice and 17b binding. Other possible mechanism in achieving the intermediate state may involve stabilization of the inner domain in a conformation between free and pre-bound conformation by the specific interactions between the inner domain residues (Trp112 and Met475) and derivatized D1D2, which are absent in gp120 and wild type D1D2 binding.

Earlier study has suggested that the stabilization of the Phe43 cavity in gp120 by a S375W mutation drives gp120 into a conformation similar to CD4-bound state (Xiang et al. 2002) presumably by rigidifying gp120 in providing a hydrophobic core at the nexus of inner domain, outer domain, and bridging sheet. Our study on derivatized CD4, however, indicates that filling the Phe43 cavity and CD4-binding do not work additively but rather counteractively in structuring gp120 in the conformation for co-receptor binding. This theory helps in explaining the greatly reduced capability (10% of WT) of gp120 S375W in supporting HIV-1 infection (Xiang et al. 2002). It is possible that the cavity, in addition to its presence in CD4-bound gp120, also exists in other intermediate state of gp120 between free and bound form, in which the rigidification (filling) of the cavity is preferred more than in the CD4-bound state. Potential small-molecule drugs for the cavity should introduce even less structure reorganization in free gp120 than the derivatized D1D2 and therefore may have better chance in viral neutralization, as seen with gp120 antibodies (Kwong et al. 2002).

Our lack of success in the identification of D1D2 derivatives with sub-nanomolar affinity to gp120 had been puzzling (Example II) and now becomes clearer with the help of the structural and thermodynamic studies. The usage of D1D2 scaffold, while stabilizing the cavity for targeting, reduces gp120's plasticity in binding ligands in the cavity and imposes penalty for the conversion of gp120 from the D1D2-bound conformation to a less structured state stabilized by the filling of the cavity. On the other hand, the binding of D1D2-scaffold to gp120 is also reduced, which is evidenced by the weakened hydrogen bonds at gp120-D1D2 interface. These reasons lead to overall reduction and masking of the favorable interaction of the Phe43 cavity and the ligands. Further optimization of cavity-binding ligands could benefit from exploitation of smaller CD4-like scaffold that structures gp120 less.

In summary, the structural studies of binding of gp120 with the derivatized D1D2 provides molecular basis for our previous SAR study and reveals high plasticity of gp120 in binding the cavity-targeting ligands. With help of the thermodynamic study, we conclude that cavity-filled gp120 adopts an intermediate conformation between the CD4-bound and free states even in the presence of the D1D2 scaffold. This study should benefit the future design of new cavity ligands especially with the help of smaller scaffold.

### Materials and Methods

### Protein Production and Purification

Derivatized D1D2 proteins were prepared as described in Example II. Recombinant endoglycosidase D (Endo D) (Muramatsu et al. 2001) was produced in E. coli using a periplasmic expression vector pBAD/gIII (a gift from Takashi Muramatsu) and was purified by ammonium sulfate precipitation and size exclusion chromatography. The preparation of the other reagents for forming gp120-containing complexes were similar to that described in previous studies of gp120 complexes (Kwong et al. 1998; Kwong et al. 1999; Kwong et al. 2000). Brief descriptions of the procedures are listed below.

The human monoclonal antibodies of gp120, 17b and F105, were produced with both in-house cell culture and ascites (Strategic BioSolutions) (both hybridoma cell lines are provided by R. Wyatt) and then purified by protein-A affinity chromatography. Fab fragment of 17b were generated by papain digestion. Briefly, 17b was first reduced by 50 mM DTT for 1 h at 37° C. then dialyzed into 100 volumes of 20 mM HEPES, pH7.8, 350 mM NaCl at 4° C. for 1 h to decrease DTT concentration to 0.5 mM. Alkylation of 17b by iodoacetamide was achieved by further dialyzing the antibody into 100 volumes of 20 mM HEPES, pH7.8, 350 mM NaCl and 4 mM iodoacetamide for 24 hr at 4° C. An additional dialysis with same alkylating buffer that is devoid of iodoacetamide (overnight, 4° C.) was used for removal of extra iodoacetamide. Alkylated 17b was then concentrated and digested using ImmunoPure Fab Preparation Kit (PIERCE). The product of digestion was further purified by size exclusion chromatography on S-200 column (Pharmacia).

Recombinant gp120 core (Δ82 deltaV1/V2*ΔV3ΔC5) (83-127 GAG 195-297 GAG 330-492) (Kwong et al. 1999) from laboratory-adapted HXBc2 strain and primary isolate YU2 strain were produced in Drosophila Schneider 2 (S2) cells (obtained from R. Wyatt) under the control of an inducible metallothionein promoter as described previously (Wu et al. 1996). Briefly, the S2 cells in suspension culture were grown in protein-free medium (Insect express media from BioWhittaker), 5% fetal bovine serum and 300 μg/ml hygromycin B (Roche Diagnostic) and expression of core gp120 proteins was induced by addition of 750 mM CuSO₄ for 7 days at 25° C. Affinity chromatography was used for purification of gp120 proteins by passing cell supernatants over a F105-sepharose column.

The affinity column was then extensively washed with PBS/0.5 M NaCl. gp120 proteins were then eluted with 100 mM glycine.HCl, pH 2.8, followed by immediate neutralization with 1M Tris, pH 11. The core gp120 proteins were concentrated to 2 mg/ml (determined by 280 nM absorbance), treated with protease inhibitor cocktail (Roche) and stored in −80° C.

### Preparation and Crystallization of Ternary Complex

The preparation and crystallization of the ternary complexes composed of HXBc2 core gp120, 17b Fab and derivatized D1D2F43C were similar as described previously (Kwong et al. 1998; Kwong et al. 1999) except D1D2 was substituted with each of the four derivatized D1D2F43C proteins, including D1D2F43C-SNS-10, D1D2F43C-SNS-14, D1D2F43C-SNS-40 and D1D2F43C-DN-234. Although gp120 and 17b Fab were produced and purified almost identically as described previously, derivatized D1D2F43C proteins were produced from different resource and have different N- and C-termini from D1D2 proteins used for previous studies (Kwong et al. 1998; Kwong et al. 1999). As discussed in Example II, derivatized D1D2F43C proteins were expressed in E. coli and refolded, whereas previous studies used D1D2 expressed as soluble protein from CHO cells. Derivatized D1D2F43C proteins also have one additional Gly at its N-terminal compared to D1D2 used before and lack the two non-CD4 C-terminal residues that D1D2 has. A flow chart of the preparation is shown in FIG. 33.

Briefly, gp120 was first deglycosylated by endoglycosidase D (50 ug recombinant Endo D per 1 mg gp120) and endoglycosidase H_{f} (Endo H_{f}) (New England BioLabs) (45 unit per 1 ug gp120) at 20° C. for 6-12 hours at pH 6.0. One of the derivatized D1D2F43C proteins was then added to the solution at a molar ratio of 120:100 for stabilizing the deglycosylated gp120. Concanavalin-A (Con A) column (Sigma) was then used for removing any glycosylated gp120 from the deglycosylated ones. These complex of gp120 and derivatized D1D2F43C was then purified by size exclusion chromatography on a Superdex 200 16/60 (Pharmacia), by which Endo D and free D1D2F43C derivative were separated from the complex of gp120 and D1D2F43C-derivative. Extra Fab fragment of 17b and more derivatized D1D2F43C were further added to the binary complex and the protein mixture was again purified on a Superdex 200 16/60 to isolate free 17b Fab, D1D2F43C derivative and Endo H_{f} from the final ternary complex, which was then concentrated by Amicon Ultra-15 30K concentrator (Millipore) to a final concentration of 6-15 mg/ml and either stored at −80° C. or used for crystallization. The whole purification processes of the complexes were monitored by SDS-PAGE (FIG. 33). And the purities of the ternary complexes were also confirmed by SDS-PAGE and mass spectrometry (Columbia/HHMI Protein Core Facility).

Crystallization of the ternary complexes of HXBc2 gp120:17b:D1D2F43C-derivatives was carried out using vapor-diffusion in hanging-drop method as described previously for the complex of HXBc2 gp120, 17b and wild type D1D2 (Kwong et al. 1998; Kwong et al. 1999). Briefly, a droplet containing 0.5 μl of protein and 0.5 μl precipitation solution was composed on glass coverslip and suspended over 0.5 ml reservoir solution in a sealed well over time at 20° C., rendering increased concentrations of both protein and precipitant in the droplet and ultimately the formation of the crystals (McPherson 1999). Only crystal showers were obtained by this approach and subsequently microseeding technique (McPherson 1999) was used for producing crystals suitable for X-ray analysis.

The largest crystals obtained for all four complexes in this study were needles with cross-section about 25 μm by 25 μm (FIG. 34). These crystals normally reached their maximum sizes 1 to 2 months after microseeding. The best precipitation solutions for microseeding of the four complexes were found to be the following: for HX-SNS-10 (HXBc2 gp120:17b Fab:D1D2F43C-SNS-10): 100 mM NaCitrate pH 5.6, 7.5% isopropanol and 7.5% PEG 4000; for HX-SNS-14 (HXBc2 gp120:17b Fab:D1D2F43C-SNS-14): 30 mM NaCitrate pH 5.6, 6% isopropanol and 6% PEG 4000; for HX-SNS-40 (HXBc2 gp120:17b Fab:D1D2F43C-SNS-40): 100 mM NaCitrate pH 5.6, 7.95% isopropanol and 8% PEG 4000; for HX-DN-234 (HXBc2 gp120:17b Fab:D1D2F43C-DN-234): 35 mM NaCitrate pH 5.6, 7% isopropanol and 7% PEG4000. The reservoir solution for each complex was the same as its precipitation solution with the addition of 350 mM NaCl to compensate the high salt in the protein solution (350 mM NaCl, 5 mM Tris.HCl, pH 7.0) (Kwong et al. 2000).

YU2 core gp120 instead of HXBc2 core gp120 was also tried out in forming similar ternary complexes as described above. The preparation of the complexes was similar as described previously (Kwong et al. 2000). Three complexes were formed: YU-WT (YU2 gp120:17b Fab:D1D2), YU-SNS-10 (YU2 gp120:17b Fab:D1D2F43C-SNS-10) and YU-SNS-40 (YU2 gp120:17b Fab:D1D2F43C-SNS-40). Unfortunately, none of these complexes was able to crystallize isomorphously with the originally reported YU-WT (Kwong et al. 2000). A different crystal of YU-WT with much smaller unit cell was able to form under the similar crystallization condition published for the original YU-WT complex (FIG. 35). It had din of 2.5-3 Å but the unit cell was too small for the whole complex. All three complexes underwent extensive crystallization trial and only a few conditions were identified for crystallization of YU-SNS-10 (FIG. 35). All the crystals formed, however, diffracted poorly.

### Data Collection, Structure Determination and Refinement

Crystals of the ternary complexes of HXBc2 gp120 with 17b and derivatized D1D2 proteins were crosslinked, stabilized and flash-frozen at 100 K similarly as reported previously (Kwong et al. 1998; Kwong et al. 1999; Kwong et al. 2000). Briefly, the crystals were crosslinked by vapor diffusion using 25 μl of 1% glutaraldehyde (Sigma) in a crystallization bridge (Hampton Research) placed in the reservoir containing 500 μl of reservoir solution (see above) for 1 h at room temperature. They were then washed by reservoir solution and transferred to stabilizing solution (10% ethylene glycol, 10% 1,6-hexanediol, 10% PEG 4k, 100 mM Na₃Citrate, pH 5.6, 2.5% 2R, 3R butanediol and 2.5% sucrose). Right before data collection, paratone-N (Hampton Research) was used for replacing the external liquid surrounding the crystals, which were then immediately mounted in cryoloop of 20 μm diameter with loop diameter of 0.05 mm (Hampton Research) and flash-frozen in liquid nitrogen.

X-ray diffraction data were collected either at beamline X4A of the National Synchrotron Light Source, Brookhaven National Laboratory (HX-14 and HX-DN234 complexes) or at beamline 191D of the Advanced Photon Source (APS), Argonne National Laboratory (HX-10 and HX-40 complexes). 100-180 degrees of oscillation data were collected for each complex with half degree oscillation per image to avoid overlapping spots due to high mosaicity (0.8°-1.5°) and large unit cell dimension. The HKL-2000 program package (Minor 1997) was used for data processing and reduction.

All four structures were solved by “rigid body” replacement using the HX-wt (HXBc2 gp120:17b Fab:D1D2) complex (PDB-ID: 1G9M) as a starting model in which the Phe43 of D1D2 was mutated to Cys43, i.e. the mutated (F43C) starting model was rigid-body refined against the data of the new complexes. Torsional angle-simulated-annealing protocol, carried out by CNS (Brunger et al. 1998), was then used for further refinement of the models and then the initial Fo-Fc maps were generated and used for manual building of the compounds introduced by the derivatization of D1D2F43C, O software (Jones et al. 1997) and Coot (Emsley et al. 2004) were used for building/rebuilding of all models throughout. The new models were then further refined using CNS (simulated annealing, positional refinement, individual isotropic B value refinement, and automatic water pick and deletion) (Brunger et al. 1998) ARP-wARP (Perrakis et al. 1997) and Refmac5 (Murshudov et al. 1997) of the CCP4 program suite (CCP4 1994) combined with manual rebuilding until the R_{free} value converged. At the later stage of the refinement, the sequences of 17b antibody were also corrected based on a newer structure of HX-WT (PDB-ID: 1RZJ) and all four structures were refined. Data collection and refinement statistics are given in Table 3.5. The final models of four complexes all contains residues 2-181 of D1D2F43C plus corresponding modifications on Cys43 and residues 1-214 for the light chain of Fab fragment of 17b. A few sections in gp120 and 17b heavy chains were not built in the final models due to lack of the electron density. Among residues 1-228 in the heavy chain of 17b, residues 143-147, 142-147, 143-147 and 142-148 are missing in HX-SNS-10, HX-SNS-14, HX-SNS-40 and HX-DN-234 respectively. The residues of gp120 built in these four complexes are 84-126:196-299:329-397:410-491, 85-126:196-299:329-397:410-459:463-491, 84-126:196-299:329-397:410-460:463-491, and 85-126:196-299:329-397:410-459:464-491 respectively. The modification group on the Cys43 of each complex is named PAM (N-phenyl-acetamide) for HX-SNS-10, NYA (N-naphthalen-1-yl-acetamide) for HX-SNS-14, PEM (N-phenethyl-acetamide) for HX-SNS-40, and BPS (2-(Benzyloxy-phenyl)-acetamide) for HX-DN-234.

TABLE 3.5
Crystallographic data on core HxBc2 gp120 complexes
with 17b Fab and D1D2 derivatives

| D1D2 Derivative | | | |
|---|---|---|---|
| D1D2F43C-10 | D1D2F43C-14 | D1D2F43C-40 | D1D2F43C-DN234 |

Diffraction Data Statistics

| | | | | |
|---|---|---|---|---|
| Space group | P222₁ | | | |
| Bragg | 3.00-50.0 | 2.80-50.0 | 2.90-50.0 | 2.60-50.0 |
| spacings (Å) | | | | |
| Unit cell | 72.0, 87.4, 196.3 | 71.8, 87.7, 196.1 | 71.6, 87.7, 196.1 | 71.5, 87.8, 195.5 |
| (a, b, c) | | | | |
| No. of unique | 25944 | 30575 | 28219 | 37721 |
| reflections | | | | |
| Redundancy^{#} | 7.2 (7.2) | 3.5 (2.5) | 6.9 (6.4) | 3.9 (3.2) |
| I/σ^{#} | 15.4 (5.2) | 12.7 (2.9) | 13.8 (4.1) | 16.0 (3.4) |
| Completeness | 99.9 (100.0) | 97.1 (88.5) | 99.8 (99.9) | 97.1 (93.3) |
| (%)^{#} | | | | |
| R_{sym} (%)*^{#} | 13.4 (42.3) | 8.9 (33.9) | 13.5 (46.1) | 8.1 (36.7) |

Refinement Statistics

| | | | | |
|---|---|---|---|---|
| Bragg | 3.00-20.0 | 2.80-20.0 | 2.90-20.0 | 2.60-20.0 |
| spacings (Å) | | | | |
| R_{Work}/R_{Free} (%)^{§} | 19.2/25.2 | 20.2/25.6 | 19.2/26.1 | 19.4/26.7 |
| No. of | 7049 | 7007 | 7035 | 6991 |
| protein atoms | | | | |
| No. of water | 100 | 137 | 140 | 245 |
| atoms | | | | |
| No. of atoms | 10 | 14 | 12 | 18 |
| in the entity | | | | |
| conjugated on | | | | |
| D1D2F43C | | | | |
| No. of other | 141 | 106 | 133 | 147 |
| atoms | | | | |
| Bond length | 0.011 | 0.008 | 0.012 | 0.012 |
| r.m.s. | | | | |
| deviation (Å) | | | | |
| Bond angle | 1.4 | 1.1 | 1.4 | 1.5 |
| r.m.s. | | | | |
| deviation (°) | | | | |
| Ramachandran | 93.2/0.1 | 94.8/0.1 | 94.5/0.0 | 95.6/0.0 |
| analysis^{&} | | | | |
| Favored/ | | | | |
| Outlier (%) | | | | |
| B factors | 32.2 | 34.6 | 23.6 | 32.3 |
| (Å²) | | | | |
| All atoms | | | | |
| Conjugated | 20.9 | 21.9 | 15.8 | 28.2 |
| entity on | | | | |
| D1D2F43C | | | | |
| Main chain | 0.788/1.150 | 0.614/0.905 | 0.726/1.186 | 0.920/1.350 |
| bond/angle | | | | |
| (r.m.s.) | | | | |
| Side chain | 1.438/2.357 | 1.131/1.825 | 1.559/2.690 | 1.917/3.109 |
| bond/angle | | | | |
| (r.m.s.) | | | | |

^{#}Numbers in parentheses represent the statistics for the data in the outer shell (10%).
*R_{sym} = Σ|I − <I>|/Σ<I>. I is the observed density and <I> is the average density from multiple symmetry-related reflections.
^{§}R = Σₕₖₗ||F_{obs}| − k|F_{calc}||/Σₕₖₗ|F_{obs}|, R_{Free} was calculated using 5% of all reflections that were never used in the refinement.
^{&}http://kinemage.biochem.duke.edu/molprobity/

### Structural Analysis

Calculation of error-scaled difference-distance matrices and identification of flexible regions in gp120 were carried out with ESCET (Schneider 2000; Schneider 2002). gp120 residues used in ESCET were sorted based on the distance of the Cα of each residue to the center of the Phe43 cavity defined by atom C4 of the phenyl ring of residue 43 in D1D2F43C-SNS-10. Superimpositions of different gp120 proteins were calculated with LSQKAB of CCP4 (CCP4 1994) suite using the invariable regions identified by ESCET. RMSDs of superimposed structures were calculated using LSQMAN (Kleywegt et al. 1997).

Volumes of the Phe43 cavity in different models were calculated using the MS program (Connolly 1993). Only gp120 and D1D2 were included for the calculation. All compounds, solvent molecules or other entities in the cavity were removed for this calculation. Phe43, water molecules #43 and #218 from the complex of gp120 and wild-type D1D2 (PDB-ID 1RZJ), onto which all other models were superimposed, were included in the coordinates for all other models for cavity calculation to ensure an unbiased volume calculation by blocking all three openings of the cavity. All structural figures were prepared using PyMOL (DeLano 2002).

### Isothermal Titration Calorimetry

Isothermal titration calorimetric experiments were performed using a high-precision VP-ITC titration calorimetric system from MicroCal Inc. (Northampton, Mass.). Direct binding to gp120 was studied in experiments where the calorimetric cell, containing 3 μM gp120, was titrated with a solution of 30 μM D1D2, D1D2F43C-SNS-10, or D1D2F43C-DN-234. All reagents were dissolved in PBS (Roche Diagnostics GmbH), pH 7.4. The binding of D1D2 or D1D2 derivatives was studied at different temperatures in the range of 15-37° C. Binding of MAb 17b to gp120 was studied by stepwise additions of 15 μM (30 μM of Fab-sites) to the calorimetric cell containing 3 μM YU2 gp120 by itself or equilibrated with 5 μM of wild-type or derivatized D1D2. The effect of 17b on the binding of derivatized D1D2 to gp120 was studied by stepwise addition of D1D2 or any of the derivatives to a mixture of gp120 and 17b. All titrations were performed by adding the titrant in steps of 10 μL. All solutions were properly degassed to avoid any formation of bubbles in the calorimeter during stirring. The heat evolved upon each injection of inhibitor was obtained from the integral of the calorimetric signal. The heat associated with binding to gp120 in the cell was obtained by subtracting the heat of dilution from the heat of reaction. The individual heats were plotted against the molar ratio and the enthalpy change, □H, and association constant, Kₐ=1/K_{d}, were obtained by non-linear regression of the data.

### REFERENCES

Binley, J. M., R. Wyatt, E. Desjardins, P. D. Kwong, W. Hendrickson, J. P. Moore and J. Sodroski (1998). “Analysis of the interaction of antibodies with a conserved enzymatically deglycosylated core of the HIV type 1 envelope glycoprotein 120.” AIDS Res Hum Retroviruses 14(3): 191-8.
Brunger, A. T., P. D. Adams, G. M. Clore, W. L. DeLano, P. Gros, R. W. Grosse-Kunstleve, J. S. Jiang, J. Kuszewski, M. Nilges, N. S. Pannu, R. J. Read, L. M. Rice, T. Simonson and G. L. Warren (1998). “Crystallography & NMR system: A new software suite for macromolecular structure determination.” Acta Crystallogr D Biol Crystallogr 54 (Pt 5): 905-21.
CCP4 (1994). “The CCP4 suite: programs for protein crystallography.” Acta Crystallogr D Biol Crystallogr 50(Pt 5): 760-3.
Chen, B., E. M. Vogan, H. Gong, J. J. Skehel, D. C. Wiley and S. C. Harrison (2005). “Determining the structure of an unliganded and fully glycosylated SIV gp120 envelope glycoprotein.” Structure 13(2): 197-211.
Clapham, P. R. and A. McKnight (2002). “Cell surface receptors, virus entry and tropism of primate lentiviruses.” J Gen Virol 83(Pt 8): 1809-29.
Connolly, M. L. (1993). “The molecular surface package.” J Mol Graph 11(2): 139-41.
DeLano, W. L. (2002). The PyMOL Molecular Graphics System, DeLano Scientific, San Carlos, Calif., USA.
Emsley, P. and K. Cowtan (2004). “Coot: model-building tools for molecular graphics.” Acta Crystallogr D Biol Crystallogr 60(Pt 12 Pt 1): 2126-32.
Huang, C. C., F. Stricher, L. Martin, J. M. Decker, S. Majeed, P. Barthe, W. A. Hendrickson, J. Robinson, C. Roumestand, J. Sodroski, R. Wyatt, G. M. Shaw, C. Vita and P. D. Kwong (2005). “Scorpion-toxin mimics of CD4 in complex with human immunodeficiency virus gp120 crystal structures, molecular mimicry, and neutralization breadth.” Structure 13(5): 755-69.
Jones, T. A. and M. Kjeldgaard (1997). “Electron-density map interpretation.” Meth. Enzymol. 277: 173-208
Kleywegt, G. J. and T. A. Jones (1997). “Detecting folding motifs and similarities in protein structures.” Meth Enzymol 277, 525-545.
Kwong, P. D., M. L. Doyle, D. J. Casper, C. Cicala, S. A. Leavitt, S. Majeed, T. D. Steenbeke, M. Venturi, I. Chaiken, M. Fung, H. Katinger, P. W. Parren, J. Robinson, D. Van Ryk, L. Wang, D. R. Burton, E. Freire, R. Wyatt, J. Sodroski, W. A. Hendrickson and J. Arthos (2002). “HIV-1 evades antibody-mediated neutralization through conformational masking of receptor-binding sites.” Nature 420(6916): 678-82.
Kwong, P. D., R. Wyatt, E. Desjardins, J. Robinson, J. S. Culp, B. D. Hellmig, R. W. Sweet, J. Sodroski and W. A. Hendrickson (1999). “Probability analysis of variational crystallization and its application to gp120, the exterior envelope glycoprotein of type 1 human immunodeficiency virus (HIV-1).” J Biol Chem 274(7): 4115-23.
Kwong, P. D., R. Wyatt, S. Majeed, J. Robinson, R. W. Sweet, J. Sodroski and W. A. Hendrickson (2000). “Structures of HIV-1 gp120 envelope glycoproteins from laboratory-adapted and primary isolates.” Structure 8(12): 1329-39.
Kwong, P. D., R. Wyatt, J. Robinson, R. W. Sweet, J. Sodroski and W. A. Hendrickson (1998). “Structure of an HIV gp120 envelope glycoprotein in complex with the CD4 receptor and a neutralizing human antibody.” Nature 393(6686): 648-59.
Leavitt, S. A., A. SchOn, J. C. Klein, U. Manjappara, I. M. Chaiken and E. Freire (2004). “Interactions of HIV-1 proteins gp120 and Nef with cellular partners define a novel allosteric paradigm.” Curr Protein Pept Sci 5(1): 1-8.
Leonard, C. K., M. W. Spellman, L. Riddle, R. J. Harris, J. N. Thomas and T. J. Gregory (1990). “Assignment of intrachain disulfide bonds and characterization of potential glycosylation sites of the type 1 recombinant human immunodeficiency virus envelope glycoprotein (gp120) expressed in Chinese hamster ovary cells.” J Biol Chem 265(18): 10373-82.
Luque, I. and E. Freire (1998). “Structure-based prediction of binding affinities and molecular design of peptide ligands.” Methods Enzymol 295: 100-27.
McPherson, A. (1999). Crystallization of Biological Macromolecules, Cold Spring Harbor Laboratory Press.
Minor, Z. O. a. W. (1997). Processing of X-ray Diffraction Data Collected in Oscillation Mode Methods in Enzymology, Volume 276: Macromolecular Crystallography, part A, p. 307-326, 1997, C. W. Carter, Jr. & R. M. Sweet, Eds., Academic Press (New York).
Modrow, S., B. H. Hahn, G. M. Shaw, R. C. Gallo, F. Wong-Staal and H. Wolf (1987). “Computer-assisted analysis of envelope protein sequences of seven human immunodeficiency virus isolates: prediction of antigenic epitopes in conserved and variable regions.” J Virol 61(2): 570-8.
Muramatsu, H., H. Tachikui, H. Ushida, X. Song, Y. Qiu, S. Yamamoto and T. Muramatsu (2001). “Molecular cloning and expression of endo-beta-N-acetylglucosaminidase D, which acts on the core structure of complex type asparagine-linked oligosaccharides.” J Biochem (Tokyo) 129(6): 923-8.
Murshudov, G. N., A. A. Vagin and E. J. Dodson (1997). “Refinement of macromolecular structures by the maximum-likelihood method.” Acta Crystallogr D Biol Crystallogr 53(Pt 3): 240-55.
Myszka, D. G., R. W. Sweet, P. Hensley, M. Brigham-Burke, P. D. Kwong, W. A. Hendrickson, R. Wyatt, J. Sodroski and M. L. Doyle (2000). “Energetics of the HIV gp120-CD4 binding reaction.” Proc Natl Acad Sci USA 97(16): 9026-31.
Pan, Y., B. Ma and R. Nussinov (2005). “CD4 binding partially locks the bridging sheet in gp120 but leaves the beta2/3 strands flexible.” J Mol Biol 350(3): 514-27.
Pantophlet, R., E. Ollmann Saphire, P. Poignard, P. W. Parren, I. A. Wilson and D. R. Burton (2003). “Fine mapping of the interaction of neutralizing and nonneutralizing monoclonal antibodies with the CD4 binding site of human immunodeficiency virus type 1 gp120.” J Virol 77(1): 642-58.
Perrakis, A., T. K. Sixma, K. S. Wilson and V. S. Lamzin (1997). “wARP: improvement and extension of crystallographic phases by weighted averaging of multiple-refined dummy atomic models.” Acta Crystallogr D Biol Crystallogr 53(Pt 4): 448-55.
Rizzuto, C. D., R. Wyatt, N. Hernandez-Ramos, Y. Sun, P. D. Kwong, W. A. Hendrickson and J. Sodroski (1998). “A conserved HIV gp120 glycoprotein structure involved in chemokine receptor binding.” Science 280(5371): 1949-53.
Schneider, T. R. (2000). “Objective comparison of protein structures: error-scaled difference distance matrices.” Acta Crystallogr D Biol Crystallogr 56 (Pt 6): 714-21.
Schneider, T. R. (2002). “A genetic algorithm for the identification of conformationally invariant regions in protein molecules.” Acta Crystallogr D Biol Crystallogr 58(Pt 2): 195-208.
Schneider, T. R. (2004). “Domain identification by iterative analysis of error-scaled difference distance matrices.” Acta Crystallogr D Biol Crystallogr 60(Pt 12 Pt 1): 2269-75.
Trkola, A., T. Dragic, J. Arthos, J. M. Binley, W. C. Olson, G. P. Allaway, C. Cheng-Mayer, J. Robinson, P. J. Maddon and J. P. Moore (1996). “CD4-dependent, antibody-sensitive interactions between HIV-1 and its co-receptor CCR-5.” Nature 384(6605): 184-7.
Wu, L., N. P. Gerard, R. Wyatt, H. Choe, C. Parolin, N. Ruffing, A. Borsetti, A. A. Cardoso, E. Desjardin, W. Newman, C. Gerard and J. Sodroski (1996). “CD4-induced interaction of primary HIV-1 gp120 glycoproteins with the chemokine receptor CCR-5.” Nature 384(6605): 179-83.
Wyatt, R., P. D. Kwong, E. Desjardins, R. W. Sweet, J. Robinson, W. A. Hendrickson and J. G. Sodroski (1998). “The antigenic structure of the HIV gp120 envelope glycoprotein.” Nature 393(6686): 705-11.
Xiang, S. H., P. D. Kwong, R. Gupta, C. D. Rizzuto, D. J. Casper, R. Wyatt, L. Wang, W. A. Hendrickson, M. L. Doyle and J. Sodroski (2002). “Mutagenic stabilization and/or disruption of a CD4-bound state reveals distinct conformations of the human immunodeficiency virus type 1 gp120 envelope glycoprotein.” J Virol 76(19): 9888-99.

### Example V

### Approaches for Future Development of gp120-CD4 Inhibitors

The CD4 scaffold approach described in previous Examples has been proved to be powerful in characterizing the Phe43 cavity and related gp120 plasticity. This approach has also significantly benefited our interactive process of screening and structure-based design of gp120-CD4 inhibitors that specifically target the Phe43 cavity. Although we still have some distance from the identification of a high-affinity small-molecule drug lead that functions as gp120-CD4 antagonist, this approach, with improvement, will continue to aid the SAR and structure-based optimization of the Phe43 cavity-targeting ligands. Eventually, our goal is to identify a compound that has high enough affinity for gp120 and will become active while scaffold-free.

Based on our understanding acquired from this study, the choice of scaffolds that rigidifies gp120 less well may be more beneficial especially for screening ligands with higher affinity to the Phe43 cavity. Also, proper identification and incorporation of fragments mimicking Phe43 and Arg59 into the ligands that bind inside the Phe43 cavity should lead to a new generation of inhibitors that targets multiple sites on gp120 with higher affinity and should advance the progress of lead discovery. In the following section, I will present the proposal and the preliminary results on both approaches (FIG. 36) for the future development of gp120-CD4 inhibitors.

### Optimization of Cavity-Targeting Ligands

Structural-activity analysis and especially the structural information on the binding of gp120 to different D1D2 derivatives have provided ample information on promising directions of ligand optimization. For examples, identification of unoccupied space in the top right corner (FIG. 25B) and lower right corner (FIG. 25A) of the Phe43 cavity in gp120 bound to D1D2F43C-DN-234 together with the finding of structure-bound water molecules nearby suggest that the substitution of nearby phenyl group by hydroxy groups may enhance the affinity; observation of multiple CH . . . O hydrogen-bonds between DN-234 and gp120 also argue for a possible optimization of the DN-234 by replacing hydrogen-bond engaging carbon atoms to stronger hydrogen donors such as NH or OH groups; the expanded water channel right next to the cavity is also a potential site for derivations on the currently identified ligands. The proposed ligand optimization could be carried out either by using D1D2F43C scaffold or if possible, alternative CD4-like scaffolds that may restrain gp120 less well.

One of the alternative scaffolds for the cavity-targeting ligand attachment is the D1D2 mutant with even weaker affinity to gp120 than D1D2F43C. Arg59 of CD4, together with Phe43, are two major determinants at CD4-gp120 interface (Kwong et al. 1998). Mutation of Arg59 to Ala or Gln reduces affinity of CD4 to gp120 by 8.8 and 2.9 fold respectively (Moebius et al. 1992; Brand et al. 1995). Thus D1D2F43C:R59A could be a better scaffold than D1D2F43C in screening of cavity ligands by restraining gp120 less.

We have expressed and purified this D1D2 mutant as described in Example II with a lower yield about 40% of that for D1D2F43C. Selected D1D2F43C:R59A derivatives have also been made using a group of compounds that have been shown to render D1D2F43C derivatives with high affinities (IC₅₀<35 nM) to gp120. IC₅₀ values for the D1D2F43C:R59A derivatives have been measured using same procedures for D1D2F43C derivatives described in Example II and were compared with the IC₅₀ values of corresponding D1D2F43C derivatives (Table 4.1 and FIG. 37). Without modification, D1D2F43C:R59A inhibits the binding of gp120 to D1D2 with IC₅₀ value 5.6-fold that of D1D2F43C. Thus, for the same modifications, an IC₅₀ ratio of 5.6 can serve as a threshold that indicates equivalent contribution to gp120 binding from Cys43-attached ligands using both scaffolds. Greater or smaller number than 5.6 should indicate that the ligands perform worse or better respectively in binding gp120 using scaffold of D1D2F43C:R59A compared to D1D2F43C.

The preliminary results showed that for the modification similarly favored on D1D2F43C scaffold, more distinct difference now is seen when they are attached to D1D2F43C:R59A, evidenced by IC50 rations ranging from 2 to 50. Encouragingly, some of the best modifications identified in D1D2F43C context, such as modification by SNS-10, SNS-12 and DN-189, were found to perform even better in D1D2F43C:R59A scaffold. In conclusion, D1D2F43C:R59A scaffold with micromolar affinity to gp120 allows better distinguishment of cavity-binding ligands and should be a good candidate for scaffolds of next generation.

TABLE 4.1
Inhibition of gp120 binding to D1D2 by D1D2F43C: R59A
derivatives.

| | | IC₅₀ of | IC₅₀ ± SD of | Ratio |
|---|---|---|---|---|
| | Structure | D1D2F43C: R59A- | D1D2F43C-R | of |
| Compound | of R | R (nM) | (nM) | IC₅₀ |
| Not modified | N/A | 1151 | 206 ± 21 | 5.6 |
| Iodoacetamide | 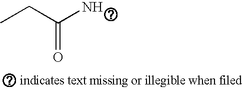 | 259 | 33.3 ± 5.5 | 7.8 |
| SNS-3 | 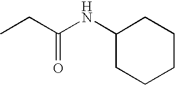 | 174 | 13.0 ± 2.1 | 13.4 |
| SNS-9 | 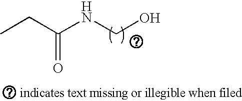 | 1049 | 33.0 ± 9.7 | 31.8 |
| SNS-10 | 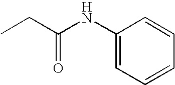 | 22.8 | 7.76 ± 0.83 | 2.9 |
| SNS-12 | 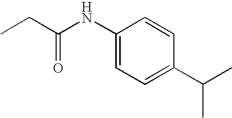 | 10.6 | 4.84 ± 0.83 | 2.2 |
| SNS-13 | 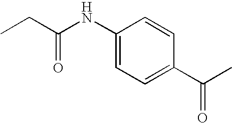 | 24.6 | 8.14 ± 0.4 | 3.0 |
| SNS-14 | 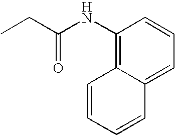 | 144 | 10.4 ± 0.8 | 13.9 |
| SNS-28 | 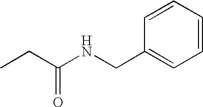 | 323 | 15.2 ± 0.7 | 21.3 |
| SNS-31 | 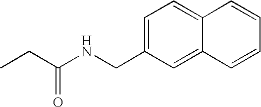 | 277 | 12.2 ± 0.8 | 22.8 |
| SNS-35 | 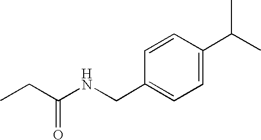 | 41 | 7.95 ± 0.09 | 5.2 |
| SNS-36 | 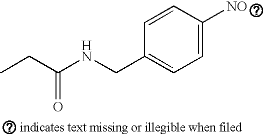 | 734 | 14.9 ± 0.0 | 49.3 |
| SNS-37 | 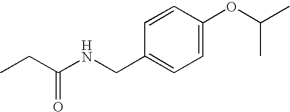 | 1223 | 25.7 ± 6.4 | 47.6 |
| SNS-40 | 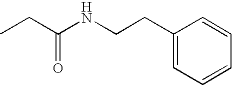 | 70 | 10.6 ± 2.9 | 6.7 |
| SNS-41 | 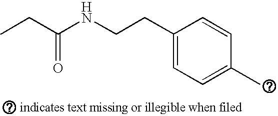 | 118 | 10.6 ± 0.7 | 11.2 |
| SNS-42 | 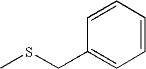 | 33 | 11.5 ± 1.8 | 2.9 |
| DN-189 | 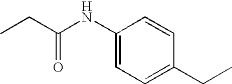 | 9 | 5.87 ± 0.17 | 1.6 |
| DN-234 | 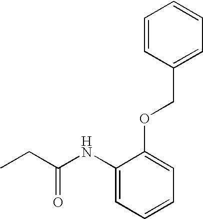 | 53 | 8.87 ± 1.09 | 6.0 |

Small peptide mimic for CD4 is another possibility for new modification scaffold. The synthesis and modification of the small peptide, however, are more difficult in general compared to that for the proteins. We had two peptides commercially synthesized (Alpha Digonostic International) to test their potentials as a scaffold (Table 4.2). G1C is designed based on peptide G1-6 (named G1F here) (Choi et al. 2001), which has been shown to inhibit gp120-CD4 binding with micromolar IC₅₀. C14Cn was designed by us to mimic the CDR2 loop of D1 domain in CD4 by using a cyclic peptide that is prone to adopt β-hairpin configuration.

We have successfully obtained the derivatives of these two peptides by compound SNS-10. Unfortunately, none of the unmodified or modified peptides displayed any inhibitory effects on gp120-CD4 binding at the concentrations of 1 mM (data not shown). In addition, G1 peptide, as the positive control peptide for G1C peptide, did not inhibit gp120-CD4 binding at 1 mM either, in contrast to the previous report (Choi et al. 2001). We conclude that the neither G1C nor C14Cn is suitable as a new modification scaffold due to little appreciable interaction with gp120. Another possible candidate of the new peptide scaffolds is CD4F33 with its Phe33 replaced by the reactive cysteine. The introduction of an additional cysteine in this mimetic that contains 3 pairs of endogenous disulfides, however, may cause problems in peptide folding.

TABLE 4.2
Structures and rational of peptides G1C and C14Cn for
cysteine-modification scaffold

| Peptide | Structure | Rational |
|---|---|---|
| G1C | PSCDLQ | PSFDLQ (G1F peptide) has been |
| | | reported to have IC₅₀ of 6 μM for |
| | | gp120-CD4 binidng.* |
| C14Cn | 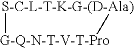 | Mimic for CDR2 loop of D1: 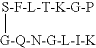 |

*See reference: (Choi et al. 2001)

### Screening of Multi-Site Targeting Ligands

In addition to the Phe43 cavity, the vestibule to the cavity (binding site for Phe43 of wild type D1D2) and the binding sites for Arg59 of wild type CD4 are also good target sites for inhibitor design. Ligands that bind more than one site mentioned above should be advantageous than the ligands for only the Phe43 cavity. Usage of F43C site of D1 for ligand attachment eliminates the possibility of full screening for the chemical groups suitable for the sites that wild type Phe43 and Arg59 bind to. Using Arg59 as tethering point for ligand screening against all three sites is a plausible idea. Consequently, we have produced D1D2F43A:R59C (F43G should be better choice). As expected, our preliminary results of using compound library designed for only targeting the Phe43 cavity did not yield derivatives with high affinity to gp120. Surprisingly, D102F43A:R59C-Iodoacetamide has an IC₅₀ value of 180 nM, which is much lower than that for unmodified D1D2F43A:R59A (601 nM) and is also comparable to that for D1D2F43C (206 nM). It suggests that the double hydrogen bonds that between Arg59 of CD4 and Asp368 of gp120 are at least partially restored by the acetamide group in D1D2F43A:R59C-Iodoacetamide. Based on preliminary modeling results on D1D2F43A:R59C, we have designed potential modification compounds composed of fragments that target either Arg59 site, the vestibule to the cavity or the Phe43 Cavity (FIG. 38).

Another more direct and possibly more risky approach for identification of multi-site targeting compounds is to screen compounds derived from the favorable ligands identified by our SAR study. The derivation could be done by attaching a chemical reactive module (e.g. sulfur, azide groups or the original bromine atom) to a cavity-favored compound (such as DN-52 or SNS-12) and then by reacting the active cavity-targeting compound with a compound library. The generated new library of compounds can be screened either for their inhibition for viral entry or for their affinities to gp120. Click chemistry could also be tried by reacting alkynes against azide-bearing cavity-targeting compounds in situ [Lewis, 2002 #4641].

It is also worth in exploring the possibility of direct attachment of our best cavity-binding group (e.g. DN-52 or SNS-12) to the identified small molecules mimicking either Phe43 or Arg59 of CD4 with the help of computational modeling. DN2149 is a small molecule designed by D. Ng and M. Head to mimic both Phe43 and Arg59 (unpublished data). It binds gp120 with nanomolar affinity. Although its binding site on gp120 has not been experimentally proved, it would be interesting to find out if it could work synergistically with the cavity-targeting groups.

In conclusion, the door to the structure-based design of HIV entry inhibitors has been opened and the search will continue.

TABLE 4.3
IC₅₀ values of D1D2F43A: R59C derivatives to gp120
binding to D1D2.

| | | IC₅₀ of |
|---|---|---|
| | | D1D2F43A: R59C- |
| Compound | Structure of R | R (nM) |
| Not modified | N/A | 601 |
| Iodoacetamide | 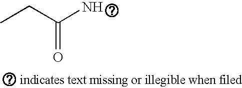 | 180 |
| SNS-1 | 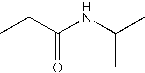 | 894 |
| SNS-8 | 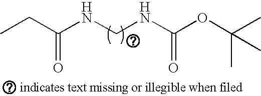 | 607 |
| SNS-10 | 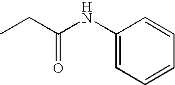 | 557 |
| SNS-13 | 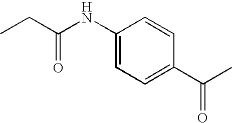 | 1306 |
| SNS-14 | 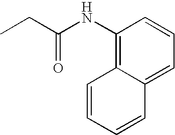 | 638 |
| SNS-16 | 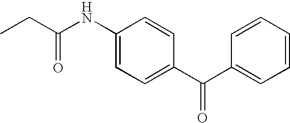 | 1157 |
| SNS-29 | 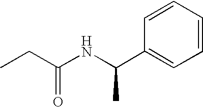 | 668 |
| SNS-31 | 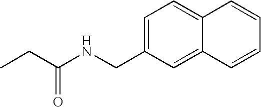 | 425 |
| SNS-42 | 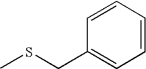 | 1091 |

### REFERENCES

Brand, D., K. Srinivasan and J. Sodroski (1995). “Determinants of human immunodeficiency virus type 1 entry in the CDR2 loop of the CD4 glycoprotein.” J Virol 69(1): 166-71.
Choi, Y. H., W. S. Rho, N. D. Kim, S. J. Park, D. H. Shin, J. W. Kim, S. H. Im, H. S. Won, C. W. Lee, C. B. Chae and Y. C. Sung (2001). “Short peptides with induced beta-turn inhibit the interaction between HIV-1 gp120 and CD4.” J Med Chem 44(9): 1356-63.
Kwong, P. D., R. Wyatt, J. Robinson, R. W. Sweet, J. Sodroski and W. A. Hendrickson (1998). “Structure of an HIV gp120 envelope glycoprotein in complex with the CD4 receptor and a neutralizing human antibody.” Nature 393(6686): 648-59.
Moebius, U., L. K. Clayton, S. Abraham, S. C. Harrison and E. L. Reinherz (1992). “The human immunodeficiency virus gp120 binding site on CD4: delineation by quantitative equilibrium and kinetic binding studies of mutants in conjunction with a high-resolution CD4 atomic structure.” J Exp Med 176(2): 507-17.

What is claimed is:

1. A soluble polypeptide consisting of a portion of CD4 comprising all HIV gp120-binding epitopes present on intact CD4, wherein the polypeptide has a cysteine substitution at a residue which, in intact CD4, interfaces with HIV gp120.

2. The polypeptide of claim 1, wherein the portion of CD4 is the portion designated D1D2.

3. The polypeptide of claim 2, wherein the cysteine substitution is an F43C or R59C substitution.

4. The polypeptide of claim 1, wherein the HIV gp120 is HIV-1 gp120.

5. A soluble polypeptide comprising (i) a portion of CD4 comprising all HIV gp120-binding epitopes present on intact CD4, wherein the polypeptide has a cysteine substitution at a residue which, in intact CD4, interfaces with HIV gp120, and (ii) a chemical moiety bound to the CD4 portion at the cysteine substitution via a thiol bond.

6. The polypeptide of claim 5, wherein the portion of CD4 is the portion designated D1D2.

7. The polypeptide of claim 6, wherein the cysteine substitution is an F43C or R59C substitution.

8. The polypeptide of claim 5, wherein the HIV gp120 is HIV-1 gp120.

9. The polypeptide of claim 5, wherein the chemical moiety is bound to the CD4 portion via reaction with a haloacetamide, a halopropanone or a 5-nitro-2-pyridinesulfenyl reagent.

10. The polypeptide of claim 5, wherein the chemical moiety is bound to the CD4 portion via reaction with 2-Bromo-N-(4-nitro-phenyl)-acetamide.

11. The polypeptide of claim 5, wherein the polypeptide binds to HIV gp120 with an IC₅₀ of ≦10 nM.

12. The polypeptide of claim 5, wherein the polypeptide binds to HIV gp120 with an IC₅₀ of ≦5 nM.

13. A method for making a derivatized soluble polypeptide comprising contacting, under suitable conditions, (a) a thiol-reactive reagent with (b) a portion of CD4 comprising all HIV gp120-binding epitopes present on intact CD4, wherein the polypeptide has a cysteine substitution at a residue which, in intact CD4, interfaces with HIV gp120.

14. The method of claim 13, wherein the portion of CD4 is the portion designated D1D2.

15. The method of claim 14, wherein the cysteine substitution is an F43C or R59C substitution.

16. The method of claim 13, wherein the HIV gp120 is HIV-1 gp120.

17. The method of claim 13, wherein the thiol reactive agent is a haloacetamide, a halopropanone or a 5-nitro-2-pyridinesulfenyl reagent.

18. A method for obtaining a structural model useful in the design of an agent for inhibiting CD4 binding to HIV gp120 comprising
(a) identifying a soluble polypeptide of claim 5 which binds to HIV gp120 with an affinity comparable to or greater than the affinity with which intact CD4 binds to HIV gp120; and
(b) obtaining a three-dimensional structure of the identified polypeptide while it is bound to HIV gp120, thereby obtaining a structural model useful in the design of an agent for inhibiting CD4 binding to HIV gp120.

19. The method of claim 18, wherein the CD4 portion of the polypeptide is the portion designated D1D2.

20. The method of claim 19, wherein the cysteine substitution in the polypeptide is an F43C or R59C substitution.

21. The method of claim 18, wherein the HIV gp120 is HIV-1 gp120.

22. The method of claim 18, wherein the chemical moiety of the polypeptide is bound to the CD4 portion of the polypeptide via reaction with a haloacetamide, a halopropanone or a 5-nitro-2-pyridinesulfenyl reagent.

23. The method of claim 18, wherein the polypeptide binds to HIV gp120 with an IC₅₀ of ≦10 nM.

24. The method of claim 18, wherein the polypeptide binds to HIV gp120 with an IC₅₀ of ≦5 nM.
